SEQUENCE LISTING (201 sequences)

SEQ ID NO: 1 - DNA, 5125 bp, Homo sapiens
tgggggcttatttggcctccccaggatggactggagcatcagtagtgcccgggtttatcacaggacagatactcaagacactcttatcaccattaggtggacttggaggagcaaaaaacaagtgcagtggctcctcagcagagacactcctgaatgtatagacatgggaaccaccttcagcatcaaaaaaggaaacgttctgcatgcccatatccagaaaaatccccactcgctgtaacttgcggtctacgaagaggaaagtcagcggcaccgtgctggcagagaggcggcttccatccctcaaactcacagtccagaatccacgctctgtggtcagatggatcctccctttgcggtgaacagattctctgcagactcccaggtcccattctgtgcttgttcccacgtccacctcccagtagtggcggccacaggtaaagcgaggggagcccaggatgcaaatggacacgtcaaatctctcggcaaggtcttgccgattctgtgtgatgcacccacttcggacgctcctgaggtcgtcagaaatgaggaggaagttgttggctgtgtcggcatccaaggtcatatccactgtgaaaaggaaaaaaagttgatcagaaaatggacagaagccaaccccatgcctctcctctacttcaaaggcccaaatatctcttgactttggtttctttaattctcttcttcccccaaaatcaaaacttttcatgaggcaacttccctgactagttcaaattctcataggtatgctttgtggcaatacccaactcttattcacagtagcaattattttactttatgtcacgtgtttgcttcattggacttttcttcatttagagtggagggtctctgaaggcagacaccatgaccccctttctaccaccttgatgcatgaaaaaatgaatgataattaagacaatgcaaatggtcgatgttaaatatgtttctgcttccatcttgtattgctcaactcagtatatagagcatgattccaggcctttgttttccatgtttgtaagcaaattaattgtgcatggttctgacagcaggtagggtatctctgcagctgacaatgtgatgacagaggtgccggtcctggacattaggaggctccaaggccaggaggaagaggggggccttagatgaaagaatgttttccatcctacatgggaggtgaaaccttaagcaaaacaatggtaaattttggagtgaagagaaaggtatttgaaagttgtttgttggctgagcgcggtggctcacacctgtaatcccagcactttgggaggccgagacgggtggatcacgaggtcaggagatcgagaccatcctggctaacacgtgaaaccctgtctctactaaaaaacatacaaaaaaattagccggacatggtggcgggtgcctgtagtcctagctactcggtagactgaggcaggagaatggcatgaacccaggaggcggagcttgcaatgagcagagatcatgccactgcacctccagcctaggcggcagagcgagactccatctcaaaaaataaaaaaaaaaagaaagttgttcgtcataactgactgatcgtagagaataagggcagtaaatacaaaggtcaataatttaatgcattgggtatgtggttgataggtgaagctgccaattctcatggaggaggcagtgtgatctggcctaaagaaaattaattgtccagagcattctgaaaaaaaaatctacagaacatatgatccttcaagtcaaaaaactgatgatcaccaatatagattttatagattacagtaagacttgaggagactgattgctgtttgatggtgaattagggaatgaaatgagatgagtggacatggacaaggaaggaggaagagggttccaaacatgacttaggcagggtgaatctgtcctccatatttcagcagagacacagtcaggtatgggtaggtggatgagtagagatcatgggtgtcagggcagaagccaccaccatttataggtgtaattctttgcccaggctgaccataacctcttcatttaaatgtccctattccccagagttatggagagtgatagtattttctgtagccaggacccctgttgaagtccatggcacaccgatgaggagaccagcgtctgcaactcgccccactcacagaaacaaacaccataggtgctccattccaaactcttcccaaccctggggtgaggttgtttggtacagtgggtgccaatactcactgtgcattagaattaccaggcccatttaaatgtcagagtcctgggaacctttgctagagagaaattcaaattaagttgctctgtgtttttgctactcagcaaagtccccaagccagcgggatcaatcctgagacttcccagttcagcatctgcattttaacaagatccccaggtaatctgagcacactaagtttagagaatcccttgtctaacccatgacatagacatcaggtttttttttttgagacggagtctcactctgtcacccaggctggagtgcagtggtgcaatcttggctcactgcaagctccgccccccccaggttaacgccattctcctgcctcaacccccaagtagctgggactacaggcacccaccaccaagcctggctaattttttgtatttttagtagagacagggtttcaccgtgttatccaggatggtcccgatctcctgacctcgtgatccacccgcctcggcctcccaaagtgctgacattacaggcatgagccaccatgcccagccagccttttttttggagatcaccaggtgattctgtatgttgtcagggctgagaatctgatgatgaagaagcatctcaattttaggtgccagacagcagctaatccccatatggaatgaggggccagttgcacagatgatttttcctggtctggtcgtaggaagggggcagtgtatacagatccctcaccttggaacttccgcatccttgggttcatctgcagaatcttcttcagcttgggctccagttccttgatgtgggaagccagcctctctagctgccaactgggcctgattttgttcttctgagagaccatggaacagcaacagcaaagtagatcctccccatggggctccttctgcagtgaattgatgcacttgaagcagacagcgcatccacactccagggacattggtttctctagatagtctgagcagacgggacagctgcttgcttcttggaagagtgcagccatgtccactgccaggggaaaagtgcacaagggaagaaaatttccgtgaggtgaaagcctgttagttgtgacaagtgacaaccttttcatgcataaaggtattgtgtcccagctttgtcacttctagaacaagaccatgagtacaaacactcaagcacatccccccaccccccgtcttcagagatttgaagttctattgggaaagaaagtcacgaatcatcactgtgctctgaactgaggtggaggaagggtccattctactggtgacccagcaactgagcctgagccagtgacacggcagcacccatgtgaggaacagcgagctggggtcatttcacctcctcctaggggaaccccagcaagagattgattgatgaacataagaattaggggctgggaggccgggcgtggtggctcaggcctataatcccagcactttgggaggctgacgtgagtggatcatctgaggtcgtcaaaagttcgagaccagcctgatgggtgaaaccccatctctactgaaaatacaaaagaattagccaggcgtggtggcacacgcctgtaatcccagatacttgggaggctgaggcaggagaattgcttgaacctgggaggcaaaggttgcagtgagccaagatccagccattgcactccggcctggacaacagagtgagacaccatctcaaaaaaaaaaaaaaaaaaaaagagagggactggatgcagtagctcacatctgtaatcccagcactttgcgaggccaaggctggaagatcccttgagccaaggagtttgagatcagcctgggcaacacaggaagaccccctctctacaaataatagaaaaattagcagtgagcagagatcacaccaggtcactccaacctggacaacagagttaagaccctgtctcaccaaaaaaaaaaaaaaaaaaatcacatccttctccagccttggaaatgaataccaaattgactgccaccactcctagactggagatctggggatccagggtgactggttttcaactctgaggcagcactcagacagcctttgttgaattcattaattacgaatttctctcattcatcccccaaggcataacccccatttctaggcatctaaggaggaaaaacacacacacaggcatgggctcacctcttctgctggaagctcctctgtcctccagctgcttccactgggcgctcaggtcttgtgcggaaggggcacatgagggcgttttattggtgagattcccacctcccactgggtcatgcccttccacaccctctaacctaatgaggctttgatttaattataacagggaattaggtttttactggtgatatatctccagttaattgtaactttagtaaatccttcatcctgacagtgtatttttctttcatatgaagataagattaaattgcctttatattaaattaggcttaatgtactaacttcaaaagcttattttagagatgtttccatcagttgtcagtaaagataattcctgtaatatgttgtaggcttcaagctttgcttggaaacctgaagtattaagtatgattggatttgcaattttacaaaatggtttaaattcatatacatcagactcaaaggtaataggggcatgcattccattgaggaaattctagggaatttttatttattttcattggtttatttctggttttgaaaaaatttcataattcataggaaaaaattggataattactattttgacaaatgaccattaagcttcttggaccttgagtacttcacacagtgttaagataaaagtcacaatttaaaaaaaaaagatgattttcttgtgaaacttgtgttctcaaataaaaattgttacccgtgaaaaaaaaaaaa

SEQ ID NO: 2 - DNA, 586 bp, Homo sapiens
atgctctcctccacactcagggtggctgtggtgtgcgtgagcaatgtcaacaggagcatggaggcccacagcatcctcaggagaaaagggctaagtgtccggtcttttggaactgaatctcatgtgaggctaccaggaccaagacccaatcgtcctgtagtttacgattttgcaacaacatataaggagatgtacaatgacctcctcaggaaagatagagaacgctacacccgcaacggaatcttacacatcttgggaagaaatgagagaatcaagcccggtccagaaagatttcaggagtgcactgatttctttgatgtcatcttcacctgtgaggagagtgtctatgacacagtggtggaagatctgtgttccagagaacagcagacctttcagcctgtgcacgtgatcaacatggacatccaagataccctggaagatgccaccctgggagctttcctcatctgtgagatttgccagtgcctggcagcagtcagacgacatggaagacaatctggaagagctgctgttgcaaatggaggagaaggcaggaaaaagctttcttcacaccgtctgcttctactga

SEQ ID NO: 3 - DNA, 1900 bp, Homo sapiens
gagaggagcagaggtctgtagaggtagagacgtaggcttcggatcttttagaattctgctggaagtctccaagtcaagaggatctacaaagaaatactgagtggagactatactgagattctgttaaagacccacttgaattcagcccccattaggagaaactttggccggagcagccaacacatcacctggaagtcttcagactagactattgaagagtggattgtgtactgagggctcccaagtgcttccagaagccaataaaggatcacttcagtttacttcacggctaaggagtaacccttaagaaccatggccaaacgcctgcaagcagagttgtcctgtccagtttgcctggattttttctcctgttccatttctctctcttgtacacacgtgttctgctttgattgcatccagaggtatatactagaaaaccatgattttagagcgatgtgccccttgtgtcgagacgtggtgaaggtacctgctttggaagaatggcaagtgagcgtcctaacacttatgaccaagcagcacaatagccgacttgagcaaagtctgcacgtgagggaggagctccggcattttcgggaggatgtgaccctggatgcagccactgccagctccctccttgtcttctccaatgatctaagaagcgctcagtgtaagaagatccaccacgatctgacaaaagatcccaggctggcctgtgtcctgggtactccctgcttctcctccggccaacattactgggaggttgaagtgggagaggtgaagtcatggtccctgggcgtctgcaaggagccggctgacagaaagagcaatgatttattccctgagcatggcttctggatcagcatgaaggcaggagcaatccatgctaacacccacctggagagaattcctgcaagccctcgccttcgccgtgtgggaattttcctggatgctgacttagaagaaatccagttttttgatgttgacaataatgtcctcatctatacacatgatggtttcttctctttggagcttttgtgtccattcttctgtcttgagctcttgggagaaggggagagtggcaacgtcctgaccatctgcccatgagaaagtcagcccttcctagaagctttctgagaggtgaaagagaattttggcctgagaaaggtcagcatgattgaggaagagataatgtgctatagtgcaaagacttggtaaatttttaaagtagattttgtagactttgtagcaaaacaattttcggatttttggggtaaattttgtggaatttgtagctaggtaactggggtctttagggatgttattaagtactgtaagcttcagttttctagtctgtagatgcggataattgtatctcagtcaaacagctgtggtaattagagacaatactatgcctttgtcttatagtaaataacaaatagagaaatcttagattgtaagtaagctagatattaggttttgtggatagacaatatctttttcattatttcaagctgttttgtgtaattcctgataatgtctgaggaggaagaaaaattcaacagccagtgtgagttattttgttgatacagcatgaaatttcagagagacaaactgatattggggaagaactaagtttttcatttttattttctttgaaacacagccacataagttttcttgaaagacaaagaactttgaccaaaatgcattgttaatggtgattcatattcttatgggaagtgtcatttacccatctcaataattggactattgtgatttataagaattcttatcaaccatgttaactaacacatattcatcaaaaattgttttcaaggttgcttttggattttttatttgtagaatttattttcttgcaaataaatttataaagcattgg

SEQ ID NO: 4 - DNA, 585 bp, Homo sapiens
atgctctcctccacactcagggtggctgtggtgtgcgtgagcaatgtcaacaggagcatggaggcccacagcatcctcaggaaaaaagggctaagtgtccggtcttttggaactgaatctcatgtgaggctaccaggaccaagacccaatcgtcctgtagtttatgattttgcaacaacatataaggagatgtacaatgacctcctcaggaaagatagagaatgctacacccgcaacggaatcttacacatcttgggaagaaatgagagaatcaagcccggtccagaaagatttcaggagtgcactgatttctttgatgtcatcttcacctgtgaggagagtgtctatgacacagtggtggaagatctgtgttccagagaacagcagacctttcagcctgtgcacgtgatcaacatggaaatccaagataccctggaagatgccaccctgggagctttcctcatctgtgagatttgccagtgcctgcagcagtcagacgacatggaagacaatctggaggagctgctcttgcaaatggaggagaaggcaggaaaaagctttcttcacaccgtctgcttctactga

SEQ ID NO: 5 - DNA, 2260 bp, Homo sapiens
ccttgtaattcataaatctctgaaaacttaaaagtttgagcaaaagtttgtcatgtttctatgagtaatttataataaaacttgatcagaatttgtgagactaacgtttgtctttatattttccttttttttttttttttttttgagacacagtctcgctctgtcgtccaggctggagtgccgtggcgtaatctcggctcactgcaacctctgcctcctggattcaaacaattcttctgcctcagcctcctgagtagctgggattacaggaccagtgatggtatagaacactgtattagagacatggagctggggctggatgaagattccatcagtaattcaatcaacagacaagtgttatccaatcacgtctttaaatcaatcactgacatggagctggggctggatgaagattccatcagtaattcaatcaacagacaagtgttatccaatcacgtctttaaatcaatcactgatcccagcccctataaaagggagcagccttaggaggcacatcagataaacccagtgtggaaagctagtcacacatcagctcagtgttcggcccgggattacccagtcaaccaaggagcttgcagttttaaagaatccaccaactgttgaaacaaatccctagagacacaaggcaagagactgaatcatcaaagtaaagtctctctgagaattattgctaagaatggctttagatctaagaaccatatttcagtgtgaaccatccgagaataatcttggatcagaaaattcagcgtttcaacaaagccaaggacctgctgttcagagagaagaagggatttctgagttctcaagaatggtgctcaattcatttcaagacagcaataattcatatgcaaggcaggaattgcaaagactttataggatctttcactcatggctgcaaccagaaaagcacagcaaggatgaaattatttctctattagtcctggagcagtttatgattggtggccactgcaatgacaaagccagtgtgaaagagaaatggaaatcaagtggcaaaaacttggagagattcatagaagacctgactgatgacagcataaatccacctgccttagtccacgtccacatgcagggacaggaagctctcttttctgaggatatgcccttaagagatgtcattgttcatctcacaaaacaagtgaatgcccaaaccacaagagaagcaaacatggggacaccctcccagacttcccaagatacttccttagaaacaggacaaggatatgaagatgaacaagatggctggaacagttcttcgaaaactactcgagtaaatgaaaatattactaatcaaggcaatcaaatagtttccctaatcatcatccaggaagagaacggtcctaggcctgaagagggaggtgtttcttctgacaacccatacaactcaaaaagagcagagctagtcactgctagatctcaggaagggtccataaatggaatcactttccaaggtgtccctatggtgatgggagcagggtgtatctctcaaccagagcagtcctcccctgagtctgcccttacccaccagagcaatgagggaaattccacatgtgaggtacatcagaaaggatcccatggagtccaaaaatcatacaaatgtgaagaatgccccaaggtctttaagtatctctgtcacttattagctcaccagagaagacacaggaatgagaggccatttgtttgtcccgagtgtcaaaaaggcttcttccagatatcagacctacgggtgcatcagataattcacacaggaaagaagcctttcacatgcagcatgtgtaaaaagtccttcagccacaaaaccaacctgcggtctcatgagagaatccacacaggagaaaagccttatacatgtcccttttgtaagacaagctaccgccagtcatccacataccaccgccatatgaggactcatgagaaaattaccctgccaagtgttccctccacaccagaagcttcctaagctgctggtctgataatgtgtataaatatgtatgcaagtatgtatattcctatagtatttatctacttaggatataagatataatctcctgattatgctttcaatttattgtcttgcttcattaaaatgtaaggctaaggagagcatggaatttgtcagttttgttcactaaagtattccaagtggttgggaaagtggaacatttccaagaaccaataaatttctgttgaataaatgaatgaatccaaaaaaaaaaaaaaa

SEQ ID NO: 6 - DNA, 1536 bp, Homo sapiens
atgaattctggaatcttgcaagaattccagatggaactcacctgccccatctgcatgaaatacttcatagacccggtcaccgtagactgtgggcacagcttttgcaggccctgtttctacttcaactggcaagacatcccaatttttactcggtgctttgaatgcatgaagacaacatggcagagaaacctcaaaactaacattcatttgaagaagatggcttcccttgccagaaaagtcagtctctggctattcctgagctctgaggagcagatgtgtggcactcacagggagacaaagaagatgttctgtgaagtggacaagagtctgctctgtctgctgtgctccagctctcaggagcaccggtatcacagacaccatcccgttgagtgggctgctgaggaacaccgggagaagcttttaaagaaaatgcagtctttatgggaaaaagcttgtgaaaatcagagaaacctgaacatggaaaccgccagaatcagccactggaaggattacgtgaatttaaggctagaagcaattagagctgagtatgagaagatggctgcatttcaccatgaagaagaaaaacataatttgcacatgctgaaaaagaaggggaaagatatttttcatcaacttcatttaagtaaagccaaaatggctcataggagggagattttaagaggaatgtatgaggagctgaaggaaatgtgccataaaccagatgtggagctacttcagggttttggagacatattacagaggagtgagtccgtgctgttgcacatgccccagcctctgaatctagagctcagtgcagggcccatcactggactgagggacaggctcatctaattctgagtggatattactctgaataatgatgagccaacagtcgtatcttttgatgtggagatttgagaagcatgtgtattggatgtgaccgtcaaaatccaccccatatcactgcaacacctacaagttttcttgcatggggtgctcagactttcacctccagcaaatattactgggatgtccatgtgggggacacttggaattgggcttttggtgtctgtaataagtattggaaagggaagaatcagaatggcaatatatatggagaggagggactctttagtcttggatgtgtcaagaatgacattcagtgcagtccctttaccacctccccacttacactgcaatatatcccaagacctaccagcctcataggattattcctggattgtgaagctagaactgtgagcttcgttgatgttaatcaaagctgccttatatacacgatccctaattgctccttctcgcctcctgtcaggcctatctttcggtgtgttcacctctgaccagagataaatcagaaatatgttcatctgctgtgagaacccgtttactccaggaagccctcttccttgtgccttatcaaacaggacaaataggttctgttttatgtcttgaattgcctcctaatgttattaaaactcagttattgtgttactattaaaaatggtaaaaacactaaaa

SEQ ID NO: 7 - DNA, 585 bp, Homo sapiens
atgctctcctccccactcagggtggctgtggtgtgtgtgagcaacatcaacaggagcatggaggcccacagcatcctcaggagaaaagggctaagtgtccggtctttcggaactgaatctcatgtgaggctaccaggacgaagacccaatcatcctgtagtttacgattttgcaacaacatataaggagatgtacaatgacctcctcaggaaagatagagaatgctacacccacaatggaatcttacacatcttgggaagaaatgagagaatcaagcctggcccagaaagatttcaggaatgcactgaattctttgatgtcatctttacctgtgaggagcgtgtctatgacacagtggtggaagatctgtgttccagagaacagcagacctttcagcctgtgcacgtgatcaacatggacatcaaagataccctggaaggtgccatcctgggagctttcctcatctgtgagatttgccagtgcctgcagcagtccgacgacatggaagacagtctggaggagctgctgttgcaaatggaggagaaggcaggaaaaagctttcttcacaccgtctgcttctactga

SEQ ID NO: 8 - DNA, 1671 bp, Homo sapiens
tagctttgcaactggctttggggacttccgaaagctaccagcactgcactgtgagactctcatccctgagctgaattcatctgattcgacagcaagctttggtgagaacatagatatatttctgaggaaaatggactcagacttctcacatgccttccagaaggaactcgcctgtgtcatctgtttgaactacctggtagaccctgtcaccatctgctgtgggcacagcttctgcaggccctctctgaacctttcctgggaggaagcccaaagtcctgccaactgccgtgcatgcagggaaccatcacagaaaaaggacttcaaaaccaatattcttctgaagaatttagtgaccattgccagaaaagccagtctctggcaattcctgagctctgagaaacaaatatgtgggacccataggcaaacaaagaagatgttctgtgacatggacaagagtctcctctgcttgctgtgctccaactctcaggagcacggggctcacaaacactatcccatcgaagaggcagctgaggaacaccgggagaaactcttaaagcaaatgaggattttatggaaaaagattcaagaaaatcagagaaatctatatgaggagagaagaacagccttcctctggaggggcgatgtggttttacgggcacagatgatcaggaatgagtataggaagctgcatccggttctccataaggaagaaaaacaacatttagagagactgaacaaggaataccaagagatttttcagcaactccagagaagctgggtcaaaatggatcaaaagagtaaacacttaaaagaaatgtatcaggaactaatggaaatgtgtcataaaccagatgtggagctgctccaggatttgggagacatcgtggcaaggagtgagcccgtgctgctgcacatgccccagcctgtgaatccagagctcacagcagggcccatcactggactggtgtacaggctcaaccgcttccgagtggaaatttccttccattgtgaagtaaccaatcacaatatcaggctctttgaggatgtgagaagttggatgtttagacgtggacctttgaattctgacagatctgactattttgctgcatggggagccagggtcttctcctttgggaaacactactgggagctggatgtggacaactcttgtgactgggctctgggagtctgtaacgactcctggataaggaagaatagcacaatggttaactctgaggacatatttcttcttttgtgtctgaaggtggataatcatttcagtctcttgaccacctccccagagtttcctcactatatagagaaacctctgggccgggttggtgtgtttcttgattttgaaagtggaagtgtgagttttttgaatgtcaccaagagttccctcatatggagttacccagctggctccttaacttttcctgtcaggcctttcttttactctggccacagatgatcaggattaagaaaacttactgtttgggaactccatatacaagggagcccttcactgttgatacaaagaaatcatactgttcaggcttttttgtactttagtgtcacttcattttattgctattaaatagaaaatttgtaaaaagcaaattttttgtacattttcttacaattaaaataatcccttatggcccattacct

SEQ ID NO: 9 - DNA, 643 bp, Homo sapiens
tggggtcagcaggaggaactctacagctatgggagaacctgcgttcacctcttttccaagcctgcctgttctggggaagctcaaaaggaacatgatgccctgggctttacagaagaaacgagaaatccacatggccaaggcccatcggagacgagctgcgaggtctgctctccccatgagactcaccagctgcatcttccggaggccggtgacaaggatcaggtctcatcctgacaaccaggtcagacgcagaaaaggggacgagcacctggagaagccgcagcaactctgcgcctaccggagactgcaggccctgcagccctgcagcagccaaggagaaggttcaagtccactgcatttggagagcgtcttaagtatccttgcaccggggacggccggtgaatctctggacagagctggtgctgagcgtgtgcgcagcccgcttgagcccacccctgggcggtttccagctgtggcaggggggccaaccccaggaatgggttgtcagctcccaccgcccctctctggccaattggtgactcctgcagatatccggagacaggccaggagggtgaagaaagccagggagagactggccaaggccttgcaggcagacaggctggccaggcgggcagaaatgtga

SEQ ID NO: 10 - DNA, 1668 bp, Homo sapiens
tagctttgcaactggctttggggacttccgaaagctaccagcactgcactgtgagactctcatccctgagctgaatccatctgattcgacagcaagctttggtgagaacatagatatatttctgaggaaaatggactcagacttctcacatgccttccagaaggaactcacctgtgtcatctgtttgaactacctggtagaccctgtcaccatctgctgtgggcacagcttctgtaggccctgtctctgcctttcgtgggaggaagcccaaagtcctgcaaactgccctgcatgcagggaaccatcaccgaaaatggacttcaaaaccaatattcttctgaagaatttagtgaccattgccagaaaagccagtctctggcaattcctgagctctgagaaacaaatatgtgggacccataggcaaacaaagaagatgttctgtgacatggacaagagtctcctctgcttgctgtgctccaactctcaggagcacggggctcacaaacactatcccatcgaagaggcagctgaggaagaccgggagaaactcttaaagcaaatgaggattttatggaaaaagattcaagaaaatcagagaaatctatatgaggagagaagaacagccttcctcttgaggggcgatgtggttttacgggcacagatgatcaggaatgagtataggaagctgcatccggttctccataaggaagaaaaacaacatttagagagactgaacaaggaataccaagagatttttcagcaactccagagaagttgggtcaaaatggatcaaaagagtaaacacttgaaagaaatgtatcaggaactaatggaaatgtgtcataaaccagaggtggagctgctccaggatttgggagacatcgtggcaaggagtgagtccgtgctgctgcacatgccccagcctgtgaatccagagctcactgcaggacccatcactggactggtgtacaggctcaaccgcttccgagtggaaatttccttccattttgaagtaaccaatcacaatatcaggctctttgaggatgtgagaagttggatgtttagacgtggacctttgaattctgacagatctgactattttgctgcatggggagccagggtcttctcctttgggaaacactactgggagctggatgtggacaactcttgtgactgggctctgggagtctgtaacaactcctggataaggaagaatagcacaatggttaactctgaggacatatttcttcttttgtgtctgaaggtggataatcatttcaatctcttgaccacctccccagtgtttcctcactacatagagaaacctctgggccgggttggtgtgtttcttgattttgaaagtggaagtgtgagttttttgaatgtcaccaagagttccctcatatggagttacccagctggctccttaacttttcctgtcaggcctttcttttacactggccacagatgatcaggattaagaaaacttactgtttgggaactccatatacaagggagcccttcactgttgatacaaagaaatcatactgttcaggcttttttgtactttagtgtcacttcatttttattgctattaaataaaaaatttgtaaaaggcaaaactttttgtacattttcttacaattaaaataatctcttatggaccat

SEQ ID NO: 11 - DNA, 764 bp, Homo sapiens
gtggggtcagcaggaggaactctacagctatgggagaacctgcgttcacctcttttccaagcctgcctgttctggggaagctcaaaaggaacatgatgccctgggctttacagaagaaacgagaaatccacatggccaaggcccatcggagacgagctgcgaggtctgctctccccatgagactcaccagctgcatcttccggaggccggtgacaaggatcaggtctcatcctgacaaccaggtcagacgcagaaaaggggacgagcacctggagaagccgcagcaactctgcgcctaccggagactgcaggccctgcagccctgcagcagccaaggagaaggttcaagtccactgcatttggagagcgtcttaagtatccttgcaccggggacggccggtgaatctctggacagagctggtgctgagcgtgtgcgcagcccgcttgagcccacccctgggcggtttccagctgtggcaggggggccaaccccaggaatgggttgtcagctcccaccgcccctctctggccaattggtgactcctgcagatatccggagacaggccaggagggtgaagaaagccagggagagactggccaaggccttgcaggcagacaggctggccaggcgggcagaaatgtgacaggtggatgaagtgcagtcctgggttttgaatccctatcttttaatgcccgtcctctttcctgctatgaattgtcacactttgtacttccccaccagttctttattaaaagcatttgaa

SEQ ID NO: 12 - DNA, 411 bp, Homo sapiens
atggcgcgcaccaagcagaccgcccgcaaagccaccgcctggcaggcccccaggaagcccctggccaccaaagcagccggaaaaagggcgccgcctacaggagggatcaagaagcctcaccgctacaagcctggcaccctggcgctgcgggaaatcagaaagtaccagaagtccacgcagctgctcctgcgcaagctgcccttccagcgcctggtgcgcgagatcgcccaggccatcagcccggacctgcgcttccagagtgcggccattggcgccctgcaggaggccagcgaggcctacctggtgcaactctttgaagacaccaacctgtgtgccatccatgccaggcgcgtcacaattatgccccgagacatgcagctggcccgccgcctccgcagagagggtccttaa

SEQ ID NO: 13 - DNA, 585 bp, Homo sapiens
atgctctcctccacactcagggtggctgtggtgtgcgtgagcaatgtcaacaggagcatggaggcccacagcatcctcaggagaaaagggctaagtgtccggtcttttggaactgaatctcatgtgaggctaccaggaccaagacccaatcgtcctgtagtttatgattttgcaacaacatataaggagatgtacaatgacctcctcaggaaagatagagaacgctacacccgcaacggaatcttacacatcttgggaagaaatgagagaatcaagcccggtccagaaagatttcaggagtgcactgattcctttgatgtcatcttcacctgtgaggagagtgtctatgacacagtggtggaagatctgtgttccagagaacagcagacctttcagcctgtgcacgtgatcaacatggaaatccaagataccctggaagatgccaccctgggagctttcctcatctgtgagatttgccagtgcctgcagcagtcagacgacatggaagacaatctggaggagctgctcttgcaaatggaggagaaggcaggaaaaagctttcttcacaccgtctgcttctactga

SEQ ID NO: 14 - DNA, 585 bp, Homo sapiens
atgctctcctccacactcagggtggctgtggtgtgcgtgagcaatgtcaacaggagcatggaggcccacagcatcctcaggagaaaagggctaagtgtccggtcttttggaactgaatctcatgtgaggctaccaggaccaagacccaatcgtcctgtagtttatgattttgcaacaacatataaggagatgtacaatgacctcctcaggaaagatagagaacgctacacccgcaacggaatcttacacatcttgggaagaaatgagagaatcaagcccggtccagaaagatttcaggagtgcactgattcctttgatgtcatcttcacctgtgaggagagtgtctatgacacagtggtggaagatctgtgttccagagaacagcagacctttcagcctgtgcacgtgatcaacatggacatccaagataccctggaagatgccaccctgggagctttcctcatctgtgagatttgccagtgcctgcagcagtcagacgacatggaagacaatctggaggagctgctcttgcaaatggaggagaaggcaggaaaaagctttcttcacaccgtctgcttctactga

SEQ ID NO: 15 - DNA, 1488 bp, Homo sapiens
gccacgtcactggctcaggctcagctgctgggtcaccaggagaatggacccttcctccacctcagctcagagcacagtgatgattcgtgactttcccaatagaacttcaaatctctgaggacggggggtggggggatgtgcttgagtgtttgtactcatggtcttgttctcggagtgacaaagctggaacacaatacctctatgcatgaaaaggttgtcacttgtcacaactaacaggctttcacctcaaggaaattttcttcccttgtgtacttttcccctggcagtggacatggctgcactcttccaagaagcaagcagctgtcccgtctgctcagactatctggaaaaaccaatgtccctggagtgtggatgcaccgtctgcctcaagtgcatcaattcgctgcagaaggagccccatggggaggatctgctttgctgttgctgttccatggtctctcagaggaacaaaatcaggcccaatcggcagctagagaggctggtttcccacatcaaggaactggagcccaagctgaagaagattctacagatgaacccaaggatgcggaagttccaagtggatatgaccttggatgccgacacagccaacaacttcctcctcatttctgacgacctcaggagcgtccgaagtgggctcatcacacagaatcggcaagaccttgccgagagatttgacgtgtccgtttgcatcctgggctcccctcgctttacctgtggccgccactactgggaggtggacgtgggaacaagcacagaatgggacctgggagtctgcagagaatctgttcactgcaaagggaagatccagctgaccacagagcttggattctggactgtgagtttgagggatggaagccgcctctctgccagcacggtgccgctgactttcctcttagtagaccgcaagttacagcgagtggggatttttctggatatgggcatgcagaacgtttccttttttgatgctgaaagtggttcccatgtctatacattcaggagcgtctctgctgaggagccactgcgcccatttttggctccttcaattccacctaatggtgatcaaggtgtcttgagcatctgtcctttgatgaactcaggcactactgatgctccagtccgtcctggggaggccaaataagccgccactgcaaaaaaaaacagggtcagaaaattacttgggtgggtagacttaggaattttctacttggtaaaagcattatacagtcataggagaaagatatgggacatttctataatctatattctaatttgattcgattattgagtcgtaagtattaattattgccaccatccaactcattgagtcttatggttcacatcttgtttcctatagaaatgttctgtattctcggatcaatttccaaatgctttactttttcatttctgtaagttcaaatcaatgtttaaattatagaagttatgaggtaaataaacatttggatatca

SEQ ID NO: 16 - DNA, 2160 bp, Homo sapiens
acccacagcactcattcctgaagctactggttggttccctgagaggtcccagaactctgcgaagtgagtccagcgctgagattttccttgcagatctatcaggatgagcatccaggccccacccagactactggagctggcagggcagagcctgctgagagaccaggccttgtccatctctgccatggaggagctgcccagggtgctctatctcccactcttcatggaggccttcagcaggagacacttccagactctgacggtgatggttcaggcctggcccttcacctgcctccctctgggatcactgatgaagacgcttcatttggagaccttaaaagcattgctggaagggcttcatatgctgcttacacagaaggatcgccccaggaggtggaaacttcaagtgctggatttgcgggatgttgacgagaatttctgggccagatggcctggagcctgggccctgtcctgcttcccagagaccacgagtaagaggcagacagcagaggactgtccaaggatgggagagcaccagcccttaaaggtgttcatagacatctgcctcaaggaaataccccaggatgaatgcctgagatacctcttccagtgggtttaccaaaggagaggtttagtacacctgtgctgtagtaagctggtcaattatctaacgccgattaaatatctcagaaagtcattgaaaataatatacctgaatagtattcaagagctggaaattcgcaacatgtcctggccacgtctgataagaaagcttcgttgttacctgaaggagatgaagaatcttcgcaaactcgttttctccaggtgccatcattacacgtcagataatgaactccaaggacggttagttgccaaattcagctctgtgttcctcaggctggaacaccttcagttgcttaaaataaaattgatcaccttcttcagtgggcacctggaacagctgatcaggtgcctccagaaccccttggagaacttggaattaacttatggctacctattggaagaagacatgaagtgtctctcccagtacccaagcctcggttacctaaagcatctgaatctcagctacgtgctgctgttccgcatcagtcttgaacccctcggagctctgctggagaaaattgctgcctctctcaaaaccctcatcttggagggctgtcagatccactactcccaactcagtgccatcctgcctgccctgagccggtgctcccagctcaccaccttctactttggcagaaattgcatgtctattgacgccctgaaggacctgctgcgccacaccagtgggctgagcaagttaagcctggagacgtatcctgcccctgaggagagtttgaattccttggttcgtgtcaattgggagatcttcaccccacttcgggctgagctgatgtgtacactgagggaagtcaggcagcccaagaggatcttcattggccccaccccctgcccttcctgtggctcatcaccgtctgaggaactggagctccatctttgctgctagggaaggcgtgcccagtggggtagagaaatccaaagttctcttccaggcacttggacactaaaatctactatgtgggtgcaaactatttttctcttttcttatttatttcattttttaataattccaaaatttttattaaagacaatttgagacagggtttcgctgtgttgctccagctggtctcaaactgctgggcttatgggatcctcctgcctcagcttcctaaagtgctgggattactggcatgagtgactgtgtccaggccacatgcaacttaaaggaagcacaggcaagtgttcagtgtgagggaaaaaacataacagcagggggcaaggttggaggaaaatgttgaggtgacatcagtgagaacttcagggacccgtgtcctagagtcggaaagagaagctaaagttctacagtgatgagactgttatccctgcaaggatggttaccaaggaatatcagcaataaagagcacctgaatgaaaacttttaacctgttgtgcaatttatccatcagaaatctctagttatcgagttacggatggaaaaataacgaaatactaatttgtctgtgattgagtttcagttgtagaacatcaaagcaaccaaataaaaattagatcattttg

SEQ ID NO: 17 - DNA, 695 bp, Homo sapiens
gggaagctcaaaagaaacatgatgccctgggctttacagaagaaacgagaaatccacatggccaaggcccatcggagacgagctgcgaggtctgctctccccatgagactcaccagctgcatcttccggaggccggtgacaaggatcaggtctcatcctgacaaccaggtcagacgcagaaaaggggacgagcacctggagaagccgcagcaactctgcgcctaccggaaactgcaggccctgcagccctgcagcagccaaggagaaggttcaagtccactgcatttggagagcgtcttaagtatccttgcaccggggacggccagtgaatctctggacagagctggtgctgagcgtgtgcgcagcccgcttgagcccacccctgggcggtttccagctgtggcaggggggccaaccccaggaatgggttgtcagctcccaccgcccctctctggccaattggtgactcctgcagatatccggagacaggccaggagggtgaagaaagccagggagagactggccaaggccttgcaggcagacaggctggccaggcgggcagaaatgctgacaggtggatgaagctcagtcttgggctttcggtccctttcttttaatgcccatcctcattcctactctgaattgtcacacttttcccttccccaccagttctttaataaaagtatttgaaaggc

SEQ ID NO: 18 - DNA, 1648 bp, Homo sapiens
gcagggaatgagctcctgatcttggggagtacttaaaagaattttttcttggaagaattactgcaggaaacattcatagaaccttgggaaacatgaattctggaatcttgcaagtcttccagagggcactcacctgtcccatctgcatgaactacttcctagacccagtcaccatagactgtgggcacagcttttgccggccctgtttgtacctcaactggcaagacacggcagttcttgctcagtgctctgaatgcaagaagacaacgcggcagagaaacctcaacactgacatttgtttgaagaacatggctttcattgccagaaaagccagcctccggcaattccttagctctgaggagcaaatatgtgggatgcacagagagacaaagaagatgttctgtgaagtggacaagagcctgctctgtttgccgtgctccaactctcaggagcaccggaatcacatacactgtcccattgagtgggctgctgaggaacgccgggaggagctcctaaaaaaaatgcagtctttatgggaaaaagcttgtgaaaatctcagaaatctgaacatggaaaccacaagaaccagatgctggaaggattatgtgagtttaaggatagaagcaatcagagctgaatatcagaagatgcctgcatttctccatgaagaagagcaacatcacttggaaaggctgcgaaaggagggcgaggacatttttcagcaactcaatgaaagcaaagccagaatggaacattccagggagcttttaagaggaatgtatgaggatctgaagcaaatgtgccataaagcagatgtggagctactccaggcttttggagacatattacacaggtatgagtctctgctgctgcaagtgtctgagcctgtgaatccagagctcagtgcagggcccatcactggactgctggacagcctcagtggattcagagttgattttactctgcagcctgaaagagccaatagtcatatcttcctgtgtggagatttgagaagcatgaatgttggatgtgaccctcaagatgatcccgatatcactggaaaatctgaatgttttcttgtatggggggctcaggctttcacatctggcaaatattattgggaggttcatatgggggactcttggaattgggcttttggtgtctgtaacaattattggaaagagaagagacagaatgacaagatagatggagaggagggactctttcttcttggatgtgttaaggaggacactcactgcagtctctttaccacctccccacttgtggtgcaatatgttccaagacctaccagcacagtaggattattcctggattgtgaaggtagaaccgtgagctttgttgatgttgatcaaagttccctgatatacaccatccccaattgctccttctcacctcctctcaggcctatcttttgctgtagtcacttctgaccagagaaaagtcagaaatgtgcctgtatgctctgggaacctgtttatcccagaaagccctctttttcgcacctcatcaaacagaacaaataagttatatttaatgtctttagttgcattctaatgtcatcaaaactcatttatagtgtttctattaaatatggtgaaaacattaaaaaaaaaaaaaaaaaaaaaaa

SEQ ID NO: 19 - DNA, 773 bp, Homo sapiens
gtggggtcagcaagagaaactctacggctatgggagagcctgcgttcacctcttttccgagcccacctgttctggggaagctcaaaagaaacatgatgccctgggctttacagaagaaacgagaaatccacatggccaaggcccatcggagacgagctgcgaggtctgctctccccatgagactcaccagctgcatcttccggaggccggtgacaaggatcaggtctcatcctgacaaccaggtcagacgcagaaaaggggacgagcacctggagaagccgcagcaactctgcgcctaccggagactgcaggccctgcagccctgcagcagccaaggagaaggttcaagtccactgcatttggagagcgtcttaagtatccttgcaccggggacggccggtgaatctctggacagagctggtgctgagcgtgtgcgcatcccgcttgagcccacccctgggcggtttccagctgtggcaggggggccaaccccaggaatgggttgtcagctcccaccgcccctctctggccaattggtgactcctgcagatatccggagacaggccaggagggtgaagaaagccagggagagactggccaaggccttgcaggcagacaggctggccaggcaggcagaaatgctgacaggtgggtgaagctcagtcctgggctttcggtccctttcttttaatgcccatcctcattcctactctgaattgtcacacttttcccttccccaccagttctttaataaaagtatttgaaaggcaaca

SEQ ID NO: 20 - DNA, 690 bp, Homo sapiens
atggcatctcgtgtcctgtccctggatggcttaacccaggaaaactcctggatccaagaggagtcagacaaaggaatggtggccttgtgcgtgctgatattctcacagatgagtgagtccgtgctgctgcacatgccccagcctgtgaatccagagttccctacagggcccatcacaggaccggtggacaggctcaaccacttccgagtggaaatttccttcacttgggaaggaaccaattacaatatcgggctgtttgaggatgtgagaagtttgatgtttagacgtggatctttgaattctgacagatctgactattttgctgcatggggagcctgggtcttctcctctggcaaacactactgggagctggatgtggacaactcttgggactgggctctgggagtctgtaaggactcctggataaggaagaatagcacaatgtttaaatctaaggacatatttcttcttttatgtgtgaaggtggataatcatttcagtctcttgaccacctccccaatgtttcctcactatgtagagaaacctctgggccgagttggtgtgtttcttgattttgaaagtggaagtgtgagttttttgaatgtcaccaagaattccctcatatggaattacccagctggctccttaaattttcctgtcaggtctttcttttacactggccacagatga

SEQ ID NO: 21 - DNA, 652 bp, Homo sapiens
gtttgttagactttcccctattctgaatcaggtgggagcacagcatggccgtgggaacgagtgctctcagcaaggagccgtggtggaccctgcccgaaaactttcattctccaatggtgttccacatggaagaggaccaggaggagctcatcttcggacttgatgacacgtaccttcgctgcattgagctgcacagccacacccttattcagctggagaggtgtttcacagctacaggccagacacgtgtgactgtagtcggaccaccaatggcaaagcagtggctgctgctcatgttccattgcgtggggagccaggactccaagtgtcacgctcgaggtctgaagatgctagagcgtgtccgaagccagcccctgaccaatgatgacctggtcacctccgttagcctgccaccgtacaccggagactgatctcccctgggaagaggggatgagaggagaagtcttggcttcgcttttggtgagaaagacatgaaaaacactgaagaaagtttgaatttgtggccagacctgcccctctcaggccttctaggcatgtgtctgagggaagtctcctgagtgttagaagtgttattggcaaagcaatccaataaaataagctacctatgatgcaaaaaaaaaaaaaaaaaaaa

SEQ ID NO: 22 - DNA, 773 bp, Homo sapiens
gtggggtcagcaagagaaactctacggctatgggagagcctgcgttcacctcttttccgagcccacctgttctggggaagctcaaaagaaacatgatgccctgggctttacagaagaaacgagaaatccacatggccaaggcccatcggagacgagctgcgaggtctgctctccccatgagactcaccagctgcatcttccggaggccggtgacaaggatcaggtctcatcctgacaaccaggtcagacgcagaaaaggggacgagcacctggagaagccgcagcaactctgcgcctaccggagactgcaggccctgcagccctgcagcagccaaggagaaggttcaagtccactgcatttggagagcgtcttaagtatccttgcaccggggacggccggtgaatctctggacagagctggtgctgagcgtgtgcgcatcccgcttgagcccacccctgggcggtttccagctgtggcaggggggccaaccccaggaatgggttgtcagctcccaccgcccctctctggccaattggtgactcctgcagatatccggagacaggccaggagggtgaagaaagccagggagagactggccaaggccttgcaggcagacaggctggccaggcaggcagaaatgctgacatgtagatgaagcgcagtcctgggctttcggtccctttcttttaatgcccatcctcattcctactctgaattgtcacacttttcccttccccaccagttctttaataaaagtatttgaaaggcaaca

SEQ ID NO: 23 - DNA, 1463 bp, Homo sapiens
cttagagggagctgtgttttggtgacctctgaaactcagtactgcagcgaatgagctcctgaccttgaggagtacttaacagaattatgtctcgaagaatcattgtgggaacccttcaaagaacccagcgaaacatgaattctggaatctcgcaagtcttccagagggaactcacctgccccatctgcatgaactacttcatagacccggtcaccatagactgtgggcacagcttttgcaggccctgtttctacctcaactggcaagacatcccaattcttactcagtgctttgaatgcataaagacaatacagcagagaaacctcaaaactaacattcgattgaagaagatggcttcccttgccagaaaagccagtctctggctattcctgagctctgaggagcaaatgtgtggcattcacagggagacaaagaagatgttctgtgaagtggacaggagcctgctctgtttgctgtgctccagctctcaggagcaccggtatcacagacactgtcccgctgagtgggctgctgaggaacactgggagaagcttttaaagaaaatgcagtctttatgggaaaaagcttgtgaaaatcagagaaacctgaatgtggaaaccaccagaatcagccactggaaggcttttggagacatattatacaggagtgagtccgtgctgctgcacatgccccagcctctgaatctagcgctcagggcagggcccatcactggactgagggacaggctcaaccaattctgagtggatattactctgcatcacaatgaagccaacagtcatatcttccgatgtggagatttgagaagcatttgtattggatgtgaccgtcaaaatccgccccatatcactgcaacacctacaagttttcttgcatggggtgctcagactttcacctctggcaaatattactgggaggtccatgtgggggactcttggaattgggcttttggtgtctgtaataagtattggaaagggaagaatcagaatggcaatatatatggagaggagggactctttagtcttgggtgtgttaagaacgacattcagtgcagtctctttaccacctccccaattacactgcagtatgtcccaagacctaccaaccatgtagaattattcctggattgtgaagctagaactgtgagcttcgttgatgttagtcaaagctcccctatatacaccatccctaattgctccttctcacttcctctcagacctatctttttctgtattctcctctgaccagagacaaatcagaaatgtgttcatctgctgtgggaacccctttatcccagaaagccctcttccttgtgccttatcaaacaggacaaataggttcggttttatgtcttgaattgcattctaatgttattaaaactcatttattgtgttactattaaatgtagtaaaaacactaaaagtataaaaaaaaaaaaaaaaaaa

SEQ ID NO: 24 - DNA, 773 bp, Homo sapiens
gtggggtcagcaagagaaactctacggctatgggagagcctgcgttcacctcttttccgagcccacctgttctggggaagctcaaaagaaacatgatgccctgggctttacagaagaaacgagaaatccacatggccaaggcccatcggagacgagctgcgaggtctgctctccccatgagactcaccagctgcatcttccggaggccggtgacaaggatcaggtctcatcctgacaaccaggtcagacgcagaaaaggggacgagcacctggagaagccgcagcaactctgcgcctaccggagactgcaggccctgcagccctgcagcagccaaggagaaggttcaagtccactgcatttggagagcgtcttaagtatccttgcaccggggacggccggtgaatctctggacagggctggtgctgagcgtgtgcgcagcccgcttgagcccacccctgggcggtttccagctgtggcaggggggccaaccccaggaatgggttgtcagctcccaccgcccctctctggccaattggtgactcctgcagatatccggagacaggccaggagggtgaagaaagccagggagagactggccaaggccttgcaggcagacaggctggccaggcaggcagaaatgctgacatgtagatgaagcgcagtcctgggctttcggtccctttcttttaatgcccatcctcattcctactctgaattgtcacacttttcccttccccaccagttctttaataaaagtatttgaaaggcaaca

SEQ ID NO: 25 - DNA, 1786 bp, Homo sapiens
gatgtttttttctctagattcatcaggatgagcctccaggccccacctagactcctggagctggctgagcagagtctgctgagagaccgggccttggccatccccaccctggaggagctgcccagggagctctttcccccactgttcatggaggcctttaccaggagatgctgcgagaccctgacaactatggtgcaggcctggcccttcacctgccttcctctagggtccctgatgaagtcatgtaatctagagatctttcgagctgtgctggaggggcttgatgcactgcttgcccagaaggttcgccccaggcggtggaaacttcaagtgttggacttgcggaatgtggatgagaacttctggggcatatggtctggagcttctgcactctccccagaggccctgagtaagagacgaacagcagggaactgtccaaggccgggtgggcagcagcccttgatggtgatcctagacctttgcttcaagaatgggatgctggatgaatgcctcacccacttcttagagtggggcaagcagagaaaaggcttactgcacgtgtgttgcaaggagctgcagatttttggaatagccatccacaggatcatagaggtcctgaacacggtggagctagactgtatccaggaggtggaagtgtgctgcccgtgggagctgtccattcttataaggttcgccccttacctgggccagatgaggaatctccgcaaacttgttctcttcaacatccatgtctctgcctgcattcccctagacaggaaggagcagtttgtcatccagttcacctctcagttcctcaagctggactacttccagaagctttacatgcactctgtctctttcctcgaaggccacctggaccagctgctcaggtgtctccaggcccccttggagacagtcgtaatgaccgaatgcctgctgtcagagtcggacctgaagcatctctcttggtgcccgagcatccgtcagctaaaagagctagacctgaggggcatcacactgacccatttcagtcctgagcccctctcagttctgctggagcaagctgaggccaccctgcagaccctggacttagaggactgtgggatcgtggattcccaactcagcgccatcctgcctgccctgagccgctgctcccagctcagcaccttcagcttctgtgggaacctcatctccatggccgccctggagaacctgctgcgccacaccgtcgggctgagcaagctaagcctggagctgtatcctgcccctctggagagttatgatgcccagggtgctctctgctggggaagattttctcaacttggggctgagctgatgaagacactgagggacttaaggcagcccaagattattgtgttcagcactgtcccctgccctcgctgtggcatcagggcctcctatgacctggagcccagtcactgtctgttgaatgcctgctgtcagggtggatttatttaaagctttcttctggtcatttggcaactgaatcctaggccatgagtgtatgtcaaagggagcacagacccatcgtttcatatgcctgctcaatgtgaacgggaaaggaaaggggatgcaggaagggagggactgggggaaaagttgagttggagtcaataggagctttagagacctgtgtcccagagaatcagaaatgggaatctgaattgctagaatgagaatcaggtaggagagacacatgagacagttacccctgcacggatggttgtaaagaaacagtcagaaataaagggaagctgagtggaaa

SEQ ID NO: 26 - DNA, 585 bp, Homo sapiens
atgctctcctccacactcagggtggctgtggtgtgcgtgagcaatgtcaacaggagcatggaggcccacagcatcctcaggagaaaagggctaagtgtccggtcttttggaactgaatctcatgtgaggctaccaggaccaagacccaatcgtcctgtagtttacgattttgcaacaacatataaggagatgtacaatgacctcctcaggaaagatagagaacgctacacccgcaacggaatcttacacatcttgggaagaaatgagagaatcaagcccggtccagaaagatttcaggagtgcactgatttctttgatgtcatcttcacctgtgaggagagtgtctatgacacagtggtggaagatctgtgttccagagaacagcagacctttcagcctgtgcacgtgatcaacatggacatccaagataccctggaagatgccaccctgggagctttcctcatctgtgagatttgccagtgcctgcagcagtcagacgacatggaagacaatctggaagagctgctgttgcaaatggaggagaaggcaggaaaaagctttcttcacaccgtctgcttctactga

SEQ ID NO: 27 - DNA, 1345 bp, Homo sapiens
atggagttccatccatcaaccttggaagagctgtcttggaggcaagcaggtccagagccccatataccacgggacctgaaccgagagcaagcctgggcctgggcactggataacttccctgtgctgagccgccactcctgccaatgccatcagctctacgggagctctgctgggagctggcggctgcaccgcgcctccaccgggcacggagcccttcccggagatgagaggggccaaaaacggcctcgagactcccatgtcgaggaatcaaaggctgaaaccacagacgggctccagtcacctcaagaagacgcaaatgctaccgacggcgaagtccaaagctgcctgatgcagcctggagatgccagcgcctgcggaggtggaaggacacaagcccatggcgccctctgcagttcaggccgcccccggaagctgctgaaaacccagggttccatcaggaaggaacccggaaacgagaggaaccccagcctttctgcaagcctgcgacactacccgcttcaaggaacactgtgctggaaggagcctgaggcacaccagtggggaccctcattctgtccctgcggacttttacactggaactgtgccctgcacccacaagagcctggagacatttctcggactgtctgcagcatcaagatatgggcttcagagcacgtttttaagcacccgtgggaaactgttacaacagctgcaatgcagaaatatccaagccctgtgaacccgagcgtggtgtgggttgacgtgttaaacagacgtatagatccctccagagagttgcacagacttctccgcacagtgggaactgcaaaactcttactgaaaacaatggaacttaaatctactaatatttcacttacaaatatgatttcagtagatgagagatttatacacaaaccacatcatcgggacccccaaaaaactattttgactcaagaagccataatcactaagaagagaggtagcctcagcagttaccttgaaggactgatggcaagaactataccttcaaatgctaataaaggccgagaagcactgaaatgggtcatacataaattacatgttaagactgaagaattgacagcttccgccagaggaagcataagaacttcgatggcagcggcggcggcagcatttgtagagaattgataatgaaaatgtgaagacaacgtcaagtactccacgtctctccaagcgggcaatatatttattttaaaaatataaatatcttttaagtagaattttttaaagtaggctgatataagaatgtgacattacatcaaaacaaagagatgcatggcttctaaataaaagcgatcacctgaaa

SEQ ID NO: 28 - DNA, 1425 bp, Homo sapiens
atgagcatcagggccccacccagactcctggagctggcaaggcagaggctgctgagggaccaggccttggccatctccaccatggaggagctgcccagggagctcttccccacactgttcatggaggccttcagcaggagacgctgtgaaaccctgaaaacaatggtgcaggcctggcctttcacccgcctccctctagggtccctgatgaagtcgcctcatctggagtcattaaaatctgtgctggaaggggttgatgtgctgttgacccaagaggttcgccccaggcagtcaaaacttcaagtgctggacttgaggaatgtggatgagaacttctgcgacatattttctggagctactgcatccttcccggaggctctgagtcagaagcaaacagcagataactgtccagggacaggcaggcagcagccattcatggtgttcatagacctttgtctcaagaacaggacactagatgaatgcctcacccacctcttagagtggggcaagcagagaaaaggcttactgcatgtgtgttgcaaggagctgcaggtttttggaatgcccatccacagtatcatagaggtcctgaacatggtggagcttgactgtatccaggaggtggaagtgtgctgcccctgggagctgtccactcttgtgaagtttgccccttacctgggccagatgaggaatctccgcaaacttgttctcttcaacatccgtgcatctgcctgcattcccccagacaacaaggggcagttcattgcccgattcacctctcagttcctcaagctggactatttccagaatctgtctatgcactccgtctctttcctcgaaggccacctggaccagctgctcaggtgtctccaggcctccttggagatggtcgttatgaccgactgcctgctgtcagagtcggacttgaagcatctctcttggtgcccgagcatccgtcaattaaaggagctggacctgaggggtgtcacgctgacccatttcagccctgagcccctcacaggtctgctggagcaagttgtggccaccctgcagaccctggacttagaggactgtgggatcatggattcccaactcagcgccatcctgcctgtcctgagccgctgctcccagctcagcaccttcagcttctgtgggaacctcatctccatggctgcccttgagaacctgctgcgccacaccgtcgggctgagcaagctaagcctggagctgtatcctgcccctctggagagttatgacacccagggtgctctctgctgggggagatttgctgaacttggggctgagctgatgaagacaccgagggacttaaggcagcccaagatcattgtgttctgcaccgtcccctgccctcgctgtggcatcagggcctcctatgacctggagcccagtcactgcctctgttga

SEQ ID NO: 29 - DNA, 774 bp, Homo sapiens
caggagtggggtcagcaggaggaactctacagctatgggagaacctgcgttcacctcttttccaagcctgcctgttctggggaagctcaaaaggaacatgatgccctgggctttacagaagaaacgagaaatccacatggccaaggcccatcggagacgagctgcgaggtctgctctccccatgagactcaccagctgcatcttccggaggccggtgacaaggatcaggtctcatcctgacaaccaggtcagacgcagaaaaggggacgagcacctggagaagccgcagcaactctgcgcctaccggagactgcaggccctgcagccctgcagcagccaaggagaaggttcaagtccactgcatttggagagcgtcttaagtatccttgcaccggggacggccggtgaatctctggacagagctggtgctgagcgtgtgcgcagcccgcttgagcccacccctgggcggtttccagctgtggcaggggggccaaccccaggaatgggttgtcagctcccaccgcccctctctggccaattggtgactcctgcagatatccggagacaggccaggagggtgaagaaagccagggagagactggccaaggccttgcaggcagacaggctggccaggcgggcagaaatgctgacaggtggatgaagctcagtcttgggctttcggtccctttcttttaatgcccatcctcattcctactctgaattgtcacacttttcccttccccaccagttctttaataaaagtatttgaaaggc

SEQ ID NO: 30 - DNA, 2259 bp, Homo sapiens
acgtgctcccggcactggcgcgagagagcgagcgccaccgccgcgggccccgcagccgttctgcctgctgtcaccgctgcctccatcgccgacactagcgctccagctgcagccaaggccgctacgaaagcgcaggaagccctcgaggagcggctgcctgggggcgcaaggctcagggcgcacacctggtttagaagatcatgaccacatggatcatctaactaaatggtacatggggacacaatggtcctttagagaatacatctgaattgctggctaatttcttgatttgccactcaacgcaggacatcgcttgttcgtagctatcagaaccctcctgaattttccccaccatgctatctttattagctggaactcctgtcctaaaacggtccttctgttgatcctgtcagtcttacttttgaaagaagatgtccgtgggagtgcacagtccagtgagaggagggtggtggctcacatgctgggtgacatcattattggagctctcttttctgttcatcaccagcctactgtggacgaagttcatgagaggaagtgtggggcagtccgtgaacagtatggcattcagagagtggaggccatgctgcataccctggaaaggatcaattcagaccccacactcttgcccaacatcacactgggctgtgagataagggattcctgctggcattcggctgtggccctagagcagagcattgagttcataagagattccctcatttcttcggaagaggaagagggcttggtgtgctctgtggatggctcctcctcttccttctgctccaagaagcccatagtaggggtcattgggcctggttccagttctgtagccattcaggtccagaatttgctccagcttttcaacatacctcagattgcttactcagcaaccatcatggatctgagtgacaagactctgttcaaatatttcatgagggttgtgccttcagatgctcagcaggcaaggtccatggtggacatagtgaagaggtacaactggacctatgtatcagccgtacacacagaaggcaactatggagaaagtgggatggaagccttcaaagatatgtcagcgaaggaagggatttgcatcgcccactcttacaaaatctacagtaatgcaggggagcagagctttgataagctgctgaagaagctcacaagtcacttgcccaaggcccgggtggtggcctacttctgtgagggcatgacggtgagaggtctgctgatggccatgaggcgcctgggtctagtgggagaatttctgcttctgggcagggaaccagatgccatctttattgagatctcaaagaacagcatcctatgggaagacagaagaaaatgccaaggtcgcttccttcagggttttggagacatattacacagaagtgagtccgtgctgctgcacatgccccagcctctgaatctagagctcagtgcagggcccatcactggactgagggacaggctcatctaattctgagtggatattactctgcattataatgaagccaacagtcatatcttctgatgtggagatttgagaagcatttgtattggatgtgaccgtcaaaatgcgccccatatcactgcaacacctacaagttttcttgcatggggtgctcagactttcacctctggcaagtattactgggaggtccatgtgggggactcttggaattgggctttcggtgtttgtaataagtactggaaagggaagaatcagaatggcaatatatatggagaggagggactctttagtcttgggattgttaagaacgacattcagtgcagtctctttaccacctccccagttacactgcagtatgtcccaagacctaccaaccatgtaggattattcctggattgtgaagctagaactgtgagcttcgttgatgttaatcaaagctcccctatatacaccatccctaattgctccttctcacctcctctcaggcctatcttttgctgtattcatctctgaccagagacaaatcagaaatgtgtttatctgctgtgggaacccctttatcccataaagccctcttccttgtgccttatcaaacaggacaaataggttctgttttatgtcttgaattgcattctaatgttattaaaactcatttattgtgttactattaaatgtggtaaaaacactaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 31 - DNA, 1965 bp, Homo sapiens
aacagacgcgggtgggcagctcagccgcatcgctaagcccggccgcctcccaggctggaatccctcgacacttggtccttcccgccccgcccttccgtgccctgcccttccctgcccttccccgccctgccccgcccggcccggcccggccctgcccaaccctgccccgccctgccccgcccagccggccacctcttaaccgcgatcctccagtgcacttgccagttgttccggacacatagaaagataacgacgggaagagcggggcccgctttggggtccaggcaggttttggggcctcctgtctggtgggaggaggccgcagcgcagcaccctgctcgtcacttgggatggagaccggctttcccgcaatcatgtaccctggatcttttattgggggctggggagaagagtatctcagctgggaaggaccggggctcccagatttcgtcttccagcagcagcccgtggagtctgaagcaatgcactgcagcaaccccaagagtggagttgtgctggctacagtggcccgaggtcccgatgcttgtcagatactcaccagagccccgctgggccaggatcccccgcagaggacagtgctagggctgctaactgcaaatgggcagtacaggaggacctgtggccaggggatcacaagaatcaggtgttattctggatcagaaaatgccttccctccagctggaaagaaagcactccctgactgtggggtccaagagccccccaagcaagggtttgacatctacatggatgaactagagcagggggacagagacagctgctcggtcagagaggggatggcatttgaggatgtgtatgaagtagacaccggcacactcaagtcagacctgcacttcctgctggatttcaacacagtttcccctatgctggtagattcatctctcctctcccagtctgaagatatatccagtcttggcacagatgtgataaatgtgactgaatatgctgaagaaatttatcagtaccttagggaagctgaaataaggcacagacccaaagcacactacatgaagaagcagccagacatcacggaaggcatgcgcacgattctggtggactggctggtggaggttggggaagaatataaacttcgagcagagaccctgtatctggctgtcaacttcctggacaggttcctttcatgtatgtctgttctgagagggaaactgcagctcgtaggaacagcagctatgcttttggcttcgaaatatgaagagatatatcctcctgaagtagacgagtttgtctatatcaccgatgatacatacacaaaacgacaactgttaaaaatggaacacttgcttctgaaagttctagcttttgatctgacagtaccaaccaccaaccagtttctccttcagtacttgaggcgacaaggagtgtgcgtcaggactgagaacctggctaagtacgtagcagagctgagtctacttgaagcagatccattcttgaaatatcttccttcactgatagctgcagcagctttttgcctggcaaactatactgtgaacaagcacttttggccagaaacccttgctgcatttacagggtattcattaagtgaaattgtgccttgcctgagtgagcttcataaagcgtaccttgatataccccatcgacctcagcaagcaattagggagaagtacaaggcttcaaagtacctgtgtgtgtccctcatggagccacctgcagttcttcttctacaataagtttctgaatggaagcacttccagaacttcacctccatatcagaagtgccaataatcgtcataggcttctgcacgttggatcaactaatgttgtttacaatatagatgacattttaaaaatgtaaatgaatttagtttcccttagactttagtagtttgtaatatagtccaacattttttaaacaataaactgcttgtcttatgaccatgtgttaga

SEQ ID NO: 32 - DNA, 1765 bp, Homo sapiens
atggatacaaagaaacgtcggaagggccctcactgctccccagatactgagaccctgctcactcccaatgggcagatctgggagaatcggttcctgatcattggccatgtcaggaaagggcttcactgcacaaggaggtggaaacttcaagtgctggatttgcgggatgttgatgagaatttctgggccagatggcctggagcctgggccctgtcctgcttcccagagaccatgagtaagaggcagacagcagaggactgtccaaggatgggagagcaccagcccttaaaggtgttcatagacatctgcctcaaggaaataccccaggatgaatgcctgagatacctctttcagtgggtttaccaaaggagaggtttagtacacctgtgctgtagtaagctggtcaattatctaacgccgattaaacatctcagaaagtcattgaaaataatatacctgaatagtattcaacagctggaaattcgcaacatgtcctggccacgtctgataagaaagcttcgttgttacctgaaggagatgaagaatcttcgcaaactcgttttctccaggtgccatcattccatgtcagataatgaactcgaaggacggttagtcaccaaattcagctctgtgttcctcaggctggaacacctccagttgcttaaaataaaattgatcaccttcttcagtgggcacctggaacagctgatcaggtgcctccagaaccccttggagaacttggaattaacttatggctacctattggaagaggatgtgaagtgtctctcccagtacccaagcctcggttacctaaagcatctgaatctcagctacgtgctgctgttccgcatcagtcttgaacccctcggagctctgctagagaaaattgctgcctctctcgaaaccctcatcttggagggctgtcagatccactactcccaactcagtgccatcctgcctggcctgagccactgctcccagctcaccaccttctactttggcagaaattgtatgtctatgggtgccctgaaggacctgctgcgccacaccagtgggctgagcaagttaagcctggagacgtatcctgcccctgaggagagtttgaattccttggttcgtgtcaattgggagatcttcgccctacttcgggctgagctgatgtgtacactgagggaagtcaggcagcccaagaggatcttcattggtcccaccccctgcccttcctgtggctcatcaccgtctgaggaactggagctccatctttgctgctagggaaggcgtgcctagcggggtagagaaatccaaagttctcttccaggcactgggacactaaaatctactatgtaggtgcaaactatttttctcttttcttatttatttcattttttaataattccaaaatttttattaaagacaatttgagacagggtttctctgtgttgctctgggatcctcctgcctcagctgggcttatgggatcctcctgcctcagcttcctaaagtgctgggattactggcatgagtgactgtgtccaggccacatgcaacttaaaggaagcacagggaagtgctcagtgtgagggaaaaaaacataacagcagggggcaaggctggaggaaaatgttgaggtgacatcaatgagaacttcagggacccgtgtcctacagagtcggaaagagaagctaaagttctacagtgatgagaatgttatccctgcaaggatggttaccaaggaatatcagaaataaagagcacctgaatgaaa

SEQ ID NO: 33 - DNA, 482 bp, Homo sapiens
ctgcacacggttttcagcctcatgcctccgtggaacctgcctgtcaggcgggcacctggaatctcaagcaactttctcaagaagggccaaggcgttatcgtcggccacgcacaagatttctctccaaacaactcacagcattgagagaattgcttgaaaagaccatgcacccaagtttggctacaatggggaaactggcttcaaagctacaacttgatctatccgtagtaaagatctggttcaagaaccagcgtgccaaatggaagaggcagcagcggcagcaaatgcagacacggccatcactagggccagcaaaccagacaacttcagtgaagaaggaggagactccctcagccataactactgcaaacattcgtccagtaagtcctggaatctctgatgcaaatgaccatgatctacgtgagccttctggtatcaagaatcctggaggagccagcgcctctgcgagggtttcatcctgg

SEQ ID NO: 34 - DNA, 444 bp, Homo sapiens
atggcgcgcaccaagcagaccgcccgcaaagccaccgcctggcaggcccccaggaagcccctggccaccaaagccgccagaaaaagggcgtcgcctacaggagggatcaagaagcctcaccgctacaagcctggcaccctggcgctgcgggaaatcagaaagtaccagaagtccacgcagctgctcctgcgcaagctgcccttccagcgtctggtgcgtgagatcgcccaggccatcagcccggacctgcgcttccagagcgcggccattggcgccctgcaggaggccagcgaggcctacctggtgcaactctttgaagacaccaacctgtgtgccatccatgccaggcgcgtcacaattatgccccgagacatgcagctggcccgccgcctccgtggagagggtgccggagagcccacgctcctgggaaaccttgcactctag

SEQ ID NO: 35 - DNA, 1855 bp, Homo sapiens
acccaaagtcttcaagcctggagttcctgcttggttcttcctgaggactgagcaccttctagactacatccagatctgttttccctgcagattcgtgaagatgagcatccggactccacccagactcctggagcttgcagggcggagcctgctgagggaccaagccttggccatgtccaccctggaggagctgcccacagaacttttccccccactgttcatggaggccttcagcaggagacgctgtgaggccctgaagctgatggtgcaggcctggcccttccgccgcctccctctgaggcctctgataaagatgccttgtctggaggccttccaagctgtgctcgatgggctggatgcactgcttacccaaggggttcatcccaggaggtggaaacttcaagtgctggatttacaggatgtctgtgagaacttctggatggtttggtctgaagctatggcccatgggtgcttcctcaatgccaagaggaacaaaaaaccagtgcaggactgtccaaggatgagaggacagcagcccttgactgtgttcgtagaactttggctcaagaacaggactctggatgaatacctcacctgcctccttctatgggtcaagcagaggaaagatttactacacctgtgctgtaagaagctgaaaattttgggaatgcccttccgcaatatcagaagcatcctgaaaatggtgaacctagactgtatccaggaggtggaagtgaattgcaagtgggtactgcccatcctgacacagtttaccccatacctgggccacatgaggaatcttcagaagctcgttctctcccacatggatgtctctcgctacgtttccccagagcagaagaaggagattgttacccagttcaccactcagttcctcaagctgtgctgcctccaaaagctttctatgaactctgtttctttcctcgaaggccacctggaccagctgctcagctgtctgaagacctcgttaaaggtcctcacaataactaactgtgtgcttttggaatcagacttgaagcatctatcccagtgcccgagtatcagtcaactaaagaccctggacctgagtggcatcagactgaccaattacagtcttgtgcctctccaaattctcctagaaaaagttgcagccacccttgagtacctggatttagatgactgtggcatcatagactcccaagtcaacgccatcctgcctgccctgagccgctgctttgagctcaacaccttcagcttctgtggaaatcccatctccatggccaccctggagaacctgctgagccacacaatcatactcaaaaacttatgcgtggagctgtatcctgccccccgggagagttatgatgctgatggtactctctgctggagcagatttgctcaaattagggctgagctgatgaagagagtgagggacttaaggcaccccaagaggatcttgttctgtactgactgctgccctgactgtggcaacaggtcattttatgacctggaggcagatcaatgctgctgttgaatgcctgcctatttgggtggatatgtcaaacgctttcttctggacacttggaaactaaaacctaggtcttaggtacatcctatagggagcacagaacccatcatttcacacatgggctctgaaagtgggaaaggaaaggtgatcaagcaggggcaggacttgggggaagtgttgccatggattcgatgggactttggggacctgtgtcctgtagagtggaaaatgggaatttgaatgtctagagtggaggcttgagaatacttgagggagttactcttggatgcatggttgtaaagaaacaatcagaaataaaggaaaactgag

SEQ ID NO: 36 - DNA, 1496 bp, Homo sapiens
gccatgtcactggctcaggctcagctgctgggtcaccaggagaatggacgcttcctccacctcagctcagagcacagtgatgattcgtgactttcccaatagaacttcaaatctctgaagacggggggtggggggatgtgcttgagtgtttgtactcatggtcttattctcggagtgacaaagctggaacacaatacctctatgcatgaaaaggttgtcacttgtcacaactaacaggctttcacctcatggaaatttttttaccttgtgtacttttcccctggcagtggacatggctgcactcttccaagaagcaagcagctgtcccgtctgctcagactatctggaaaaaccaatgtccctggagtgtggatgcgccgtctgcctcaagtgcattaattcactgcagaaggagccccatggggaggatctactttgctgttgctcttccatggtctctcggaagaacaaaatcaggcgcaatcggcagctagagaggctggcttcccacatcaaggaactggagcccaagctgaagaagattctgcagatgaacccaaggatgcggaagttccaagtggatatgaccttggatgccaacacagccaacaacttcctcctcatttctgacgacctcaggagcgtccgaagtgggcgcatcagacagaatcggcaagaccttgccgagagatttgacgtgtccgtttgcatcctgggctcccctcgctttacctgtggccgccactgctgggaggtggacgtgggaacaagcacagaatgggacctgggagtctgcagagaatctgttcaccgcaaagggaggatccagctgaccacagagcttggattctggactgtgagtttgagggatggaggccgcctctctgccaccacggtgccgctgactttcctcttcgtagaccgcaagttacagcgagtggggatttttctggatatgggcatgcagaacgtttccttttttgatgctgaaagtggttcccatgtctatacattcaggagcgtatctgctgaggagccattgcgcccatttttggctccttcagttccacctaatggtgatcaaggtgtcttgagcatctgtcctttgatgaactcaggcactactgatgctccagtccgtcctggggaggccaaataagccctcactccaaaaaaacaaaaaacagggtaagaaaattacttgggtgggtagacttaggaacgctctacttcgtaaaagcattatacaaagtcacgggagaaaaatatgggacatttcttgattgtacttaatctaatttgattagattatagagtcctaagtattaattattgccaccatcaaactcattgagtcctatggttcacatcttgtttcctatagaaatgtcctgtattctgggatcaatttccaaatgctttacttttttatttctgcaagttcaaattaatgtattatagaagttatgagttaaataaagattggaatatcacct

SEQ ID NO: 37 - DNA, 1863 bp, Homo sapiens
acccaaagtcttcaagcctggagttcctgcttggttcttcctgaggtctgagcaccttctagactacatccagatctgttttccctgcagattcatgaagatgagcatccggactccacccagactcctggagcttgcagggcggagcctgctgagggaccaagctttggccatgtccaccctggaggagctgcccacagaacttttccccccactgttcatggaggccttcagcaggagacgctgtgaggccctgaagctgatggtgcaggcctggcccttccgccgcctccctctgaggcctctgataaagatgccttgtctggaggccttccaagctgtgctcgatgggcttgatgcactgcttacccaaggggttcgtcccaggaggtggaaactccaagtgctggatttacaggatgtctgtgagaacttctggatggtttggtctgaagctatggcccatgggtgcttcctcaatgccaagaggaacaaaaaaccagtgcaggactgtccaaggatgagaggacggcagcccttgactgtgttcgtagaactttggctcaagaacaggactctggatgaatacctcacctacctccttctatgggtcaagcagaggaaagatttactacacctgtgctgtaagaagctgaaaattttgggaatgcccttccgcaatatcagaagcatcctgaaaatggtgaacctagactgtatccaggaggtggaagtgaattgcaagtgggtactgcccatcctgacacagtttaccccatacctgggccacatgaggaatcttcagaagctcgttctctcccacatggatgtctctcgctacgtttccccagagcagaagaaggagattgttacccagttcaccactcagttcctcaagctgcgctgcctccaaaagctttatatgaactctgtttctttcctcgaaggccacctggaccagctgctcagctgtctgaagacctcgttaaaagtcctcacaataactaactgtgtgcttttggaatcagacttgaagcatctatcccagtgcccgagtatcagtcaactaaagaccctggacctgagtggcatcagactgaccaattatagtcttgtgcctctccaaattctcctagaaaaagttgcagccacccttgagtacctggatttagatgactgtggcatcatagactcccaagtcaacgccatcctgcctgccctgagccgctgctttgagctcaacaccttcagcttctgtggaaatcccatctgcatggccaccctggagaacctgctgagccacacaatcatactcaaaaacttatgtgtggagctgtatcctgccccccgagagagttatggtgctgatggtactctctgctggagcagatttgctcaaattagggctgagctgatgaacagagtgagggacttaaggcaccccaagaggatcttgttctgtactgactactgccctgactgtggcaacaggtcattttatgacctggaggcagatcaatactgctgttgaatgcctgcctatttggatgggtatgtcaaacgctttcttctggacacttggaaactaaaacctaggtcttaggtacatcctaaagggagcacagaacccatcatttcacacataggctctgaaagtgggaaaggaaagctgatcaagcaggggccggacttgggggaaatgttgccatggattcgatgggactttggggacctgtgtcctgtagattcgaaaatgggaatctgaatgtctagagtggaattcaggcttgagaatacatgagggagttactcttgcatggatggttgtaaagaaacaatcagaaataaaggaaaactgagcag

SEQ ID NO: 38 - DNA, 1474 bp, Homo sapiens
atgaattctggaatctcgcaagtcttccagagggaactcacctgccccatctgcctgaactacttcatagacccggtcaccatagactgtgggcacagcttttgcaggccctgtttctacctcaactggcaagacatcccaattcttactcagtgctttgaatgcctaaagacaacacagcagagaaacctcaaaactaacattcgattgaagaagatggcttcccgtgccagaaaagccagtctctggctattcctgagctctgaggagcaaatgtgtggcactcacagggagacaaagaagatattctgtgaagtggacaggagcctgctctgtttgctgtgctccagctctctggagcaccggtatcacagacactgtcccgctgagtgggctgctgaggaacaccgggagaagcttttaaagaaaatgcagtctttatgggaaaaagtttgtgaaaatcagagaaacctgaacgtggaaaccaccagaatcagccactggaaggattatgtgaatgtaaggctagaagctattagagctgagtatcagaagatgcctgcatttcatcatgaagaagaaaaacataatttggagatgctgaaaaagaaggggaaagatatttttcatcgacttcatttaagtaaagccaaaatggctcacaggagggagattttaagaggaacgtatgcggagctgatgaaaatgtgccataaaccagatgtggagctacttcaggcttttggagacatattacacaggagtgagtccgtgctgctgcacatgccccagcctctgaatctagagctcagggcagggcccatcactggactgagggacaggctcaaccaattccgagtggatattactctgcctcataatgaagccaacagtcatatcttccgacgtggagatttgagaagcatttgtattggatgtgaccgtcaaaatgcgccccatatcactgcaacacctacaagttttcttgcatggggtgctcagactttcacctctggcaaatattactgggaggtccatgtgggggactcttggaattgggcctttggtgtctgtaataagtattggaaagggacgaatcagaatggcaatatacatggagaggagggactctttagtcttgggtgtgttaagaatgacattcagtgcagcctctttaccacctccccacttacactgcagtatgtcccaagacctaccaaccatgtaggattattcctggattgtgaagctagaactgtgagcttcgttgatgttaatcaaagctcccctatacacaccatccctaattgctccttctcacctcctctcaggcctatcttttgctgtgttcacctctgaccagagacaaatcagatatgtgttcatctgctgtgggaacccctttatcccagaaagccctcttccttgtgccttatcaaacaggacaaatacgttctgttttatgtcttgaatt

SEQ ID NO: 39 - DNA, 1840 bp, Homo sapiens
ttcctgcttggttcttcctgagatctgagcaccttctagactacatccagatctgttttccctgcagattcatgaagatgagcatctggactccacccagactcctggagcttgcagggcggagcctgctaagggaccaagctttggccatgtccaccctggaggagctgcccacagaacttttccccccactgttcatggaggccttcagcaggagacgctgtgaggccctgaagctgatggtgcagtcctggcccttccgccgcctccctctgaggcctctgataaagatgccttgtctggaggccttccaagctgtgctcgatgggctggatgcactgcttaacctaggggttcgtcccaggaggtggaaacttcaagtgctggatttacaggatgtctgtgagaacttctggatggtttggtctgaagctatggcccatgggtgcttcctcaatgccaagaggaacaaaaaaccagtggaggactgtccaaggatgagaggacggcagcccttgactgtgttcgtagaactttggctcaagaacaggactctggatgaatacctcacctgcctccttctatgggtcaagcagaggaaagatttactacacctgtgctgtaagaagctgaaaattttgggaatgcccttccgcaatatcagaagcatcctgaaaatggtgaacctagactgtatccaggaggtggaagtgaattgcaagtgggtactgcccatcctgacacagtttaccccatacctgggccacatgaggaatcttcagaaactcattctctcccacatggatgtctctcgctacgtttccccagagcagaagaaggagattgttacccagttcaccactcagttcctcaagctgcgctgcctccaaaagctttatatgaactctgtttctttcctcgaaggccacctggaccagctgctcagctgtctgaagacctcgttaaagttcctcacaataactaactgtgtgcttttggaatcagacttgaagcatctatcccagtgcccgagtatcagtcaactaaagaccctggacctgagtggcatcagactgaccaattacagtcttgtgcctctccaaattctcctagaaaaagttgcagccacccttgagtacctggatttagatgactgtggcatcatagactcccaagtcaacgccatcttgcctgccctgagccgctgctttgagctcaacaccttcagcttctgtggaaatcccatctgcatggccaccctggagaacctgctgagccacacaatcatactcaaaaacttatgcgtggagctgtatcctgccccccgggagagttatggtgctgatggtactctctgctggagcagatttgctcaaattagggctgagctgatgaacagagtgagggacttaaggcaccccaagaggatcttgttctgtactgactactgccctgactgtggcaacaggtcattttatgacctggaggcagatcaatactgctgttgaatgcctgcctatttggatgggtatgtcaaacgctttcttctggacacttggaaactaaaacctaggtcttaggtacatcctaaagggagcacagaacccatcatttcacacataggctctgaaagtgggaaaggaaagctgatcaagcaggggccggacttgggggaaatgttgccatggattcgatgggactttggggacctgtgtcctgtagagtcgaaaatgggaatctgaatgtctagagtggaattcaggcttgagaatacatgagggagttactcttgcatggatggttgtaaagaaacaatcagaaataaaggaaaactgagcag

SEQ ID NO: 40 - DNA, 1847 bp, Homo sapiens
acccaaagtcttcaagcctggagttcctgcttggttcttcctgaggtctgagcaccttctagactacatccagatctgatgaagatgagcatccggactccacccagactcctggagcttgcagggcggagcctgctgagggaccaagctttggccatgtccaccctggaggagctgcccacagaacttttccccccactgttcatggaggccttcagcaggagacgctgtgaggccctgaagctgatggtgcaggcctggcccttccgccgcctccctctgaggcctctgataaagatgccttgtctggaggccttccaagctgtgctcgatgggcttgatgcactgcttacccaaggggttcgtcccaggaggtggaaactccaagtgctggatttacaggatgtctgtgagaacttctggatggtttggtctgaagctatggcccatgggtgcttcctcaatgccaagaggaacaaaaaaccagtgcaggactgtccaaggatgagaggacggcagcccttgactgtgttcgtagaactttggctcaagaacaggactctggatgaatacctcacctacctccttctatgggtcaagcagaggaaagatttactacacctgtgctgtaagaagctgaaaattttgggaatgcccttccgcaatatcagaagcatcctgaaaatggtgaacctagactgtatccaggaggtggaagtgaattgcaagtgggtactgcccatcctgacacagtttaccccatacctgggccacatgaggaatcttcagaagctcgttctctcccacatggatgtctctcgctacgtttccccagagcagaagaaggagattgttacccagttcaccactcagttcctcaagctgcgctgcctccaaaagctttatatgaactctgtttctttcctcgaaggccacctggaccagctgctcagctgtctgaagacctcgttaaaagtcctcacaataactaactgtgtgcttttggaatcagacttgaagcatctatcccagtgcccgagtatcagtcaactaaagaccctggacctgagtggcatcagactgaccaattatagtcttgtgcctctccaaattctcctagaaaaagttgcagccacccttgagtacctggatttagatgactgtggcatcatagactcccaagtcaacgccatcctgcctgccctgagccgctgctttgagctcaacaccttcagcttctgtggaaatcccatctgcatggccaccctggagaacctgctgagccacacaatcatactcaaaaacttatgtgtggagctgtatcctgccccccgagagagttatggtgctgatggtactctctgctggagcagatttgctcaaattagggctgagctgatgaacagagtgagggacttaaggcaccccaagaggatcttgttctgtactgactactgccctgactgtggcaacaggtcattttatgacctggaggcagatcaatactgctgttgaatgcctgcctatttggatgggtatgtcaaacgctttcttctggacacttggaaactaaaacctaggtcttaggtacatcctaaagggagcacagaacccatcatttcacacataggctctgaaagtgggaaaggaaagctgatcaagcaggggccggacttgggggaaatgttgccatggattcgatgggactttggggacctgtgtcctgtagattcgaaaatgggaatctgaatgtctagagtggaattcaggcttgagaatacatgagggagttactcttgcatggatggttgtaaagaaacaatcagaaataaaggaaaactgagcag

SEQ ID NO: 41 - DNA, 1474 bp, Homo sapiens; identical to SEQ ID NO: 38

SEQ ID NO: 42 - DNA, 2438 bp, Homo sapiens
ataagccagagcctagaccagtgagccaactgtgcgaaccagacccggcagccttgctcagttcagcatagcggagcggatccgatcggatcggagcggatcggagcacaccggagcaggctcatcgagaaggcgtctgcgagaccatggagaacggatacacctatgaagattataagaacactgcagaatggcttctgtctcacactaagcaccgacctcaagttgcaataatctgtggttctggattaggaggtctgactgataaattaactcaggcccagatctttgactacggtgaaatccccaactttccccgaagtacagtgccaggtcatgctggccgactggtgtttgggttcctgaatggcagggcctgtgtgatgatgcagggcaggttccacatgtatgaagggtacccactctggaaggtgacattcccagtgagggttttccaccttctgggtgtggacaccctggtagtcaccaatgcagcaggagggctgaaccccaagtttgaggttggagatatcatgctgatccgtgaccatatcaacctacctggtttcagtggtcagaaccctctcagagggcccaatgatgaaaggtttggagatcgtttccctgccatgtctgatgcctacgaccggactatgaggcagagggctctcagtacctggaaacaaatgggggagcaacgtgagctacaggaaggcacctatgtgatggtggcaggccccagctttgagactgtggcagaatgtcgtgtgctgcagaagctgggagcagacgctgttggcatgagtacagtaccagaagttatcgttgcacggcactgtggacttcgagtctttggcttctcactcatcactaacaaggtcatcatggattatgaaagcctggagaaggccaaccatgaagaagtcttagcagctggcaaacaagctgcacagaaattggaacagtttgtctccattcttatggccagcattccactccctgacaaagccagttgacctgccttggagtcgtctggcatctcccacacaagacccaagtagctgctaccttctttggccccttgctggagtcatgtgcctctgtccttaggttgtagcagaaaggaaaagattcctgtccttcacctttcccactttcttctaccagacccttctggtgccagatcctcttctcaaagctgggattacaggtgtgagcatagtgagaccttggcgctacaaaataaagctgttctcattcctgttctttcttacacaagagctggagcccgtgccctaccacacatctgtggagatgcccaggatttgactcgggccttagaactttgcatagcagctgctactagctctttgagataatacattccgaggggctcagttctgccttatctaaatcaccagagaccaaacaaggactaatccaatacctcttggattttatttaatgtcataatgttgtcagaataaagagaaagatgaaataatttcatttttttgtgtaacttggtatggggctggggcacagaccaagattgacatgaaaggatgtgagatcgcatgtcttgtgtgactatctgcttctcagacaagcagttaggaactgagatgagatagtatgtgagggcagcaaaggatgaagaagggcaaaatgatgaaaggtgaggtggaaagaggttatgagatggtaaagaaaagttaacttctggcacttgattgccacttctgtcaggctggtcctgcctctctcccttgccttctgattgtttcatttcctgtttatttgatcatatctgaattagttcactggttagcctcttccttagttcccacttccttaccaaagccctaattatatttcctcttgtttgccttttctctcctactcttctctaacatctgcagccacactctccattcactccatgctgacaaggcagtggcaaacacttttctctgctgccagccactccactgttgactggattgctgccagccccaggcaaacctgtgaagttgtttcatactctgcttctctttgagtgcctccttctctccttcctcttcctttctgggctccagtctttctcttcacttgtgcttgtcagaacctccctgtgatactgcctccaggcatttcccccatgttggctcaccgcactattatctttgcttatcaacttgcattcagctggctggcatgtttcaaaaccacactgccctcccaggcctgtgtgccttttgagaaagaccagtgctggatgagcctctagtaatgacaacattttagttgttagtggtataatacggaagagatattttgcacaggctgctttggagaactttcaaattatcctttgtttggtaactgacctacttaactgcccaatacaaagaaaaagcaaaaaaaaaaaaaaaaaa

SEQ ID NO: 43 - DNA, 1474 bp, Homo sapiens; identical to SEQ ID NO: 38

SEQ ID NO: 44 - DNA, 1029 bp, Homo sapiens
atggggctgggttggaaggggctaagtgtatggcagcatcttctgcatagatcccaacacacagactctctgaatcctgcccacagcctcaccaccattgctgtctggtaccagcactgcttccagttcaccaggttcagcccagtgacaaggcagaggccacagccctacaccatggctcgcattaggcagactgaccacaaagccaccgcctggcaggcccccaggaagcccctggccaccaaagtcaccggcaagagggtcctggctacacgagggatcaagaagcctcaccgccacaggcttggcaccctggcgctgcacaaaaccaggaggtaccagaagtccacacagctgcttctgcacaagctgaccttccagcgcctggagcacgagattgcccaggccatcaacccagacctgcgcttccaatgtgcggccactggcgatttcagggaaacagcgaggcctacctggtccccctctttaaagacatcagcctatgtgttatccatgccaggcgtgtcacagttatgcccagagacgccgtccgcaaaagggcgccctctagaggatggatcaagaagtctcacagctgcagacctggccccatggcgctgagtgaaatcagaagtaccacaagtccactcagctgctccagcacaagctaaccttccagcgcctggtgcgcgagatcccccggaccatcagccgggacctgggcttccagagcgtggccgttggtgctttcaggggaccaatgaggcctacctggtgcgcctctttgaagacaccaacctgtatgtcatccatgccagaagcgtcccaattatgccaagagacgtgcagctggcccacagcctccgtggagagggtgctgaagagccctcctgggaaatcttgcattgtaaatggtttcctttgcgtcctgttgtgtttttcctcttctctctgttaatgatagatatgatgttagaagttgtgttcagtatcagtatcatttcctgcacggggtctaaaagtagctaa

SEQ ID NO: 45 - DNA, 1651 bp, Homo sapiens
ggtgacctctgaaactcagtactgcggcgaatgagctcctgaccttgaggagtacttaacagaattatttctcgaagaatcattgtgggaacccttcaaagaacccagcgaaacatgaattctggaatctcgcaagtcttccagagggaactcacctgccccatctgcctgaactacttcatagacccggtcaccatagactgtgggcacagcttttgcaggccctgtttctacctcaactggcaagacatcccaattcttactcagtgctttgaatgcctaaagacaacacagcagagaaacctcaaaactaacattcgattgaagaagatggcttcccgtgccagaaaagccagtctctggctattcctgagctctgaggagcaaatgtgtggcactcacagggagacaaagaagatattctgtgaagtggacaggagcctgctctgtttgctgtgctccagctctctggagcaccggtatcacagacactgtcccgctgagtgggctgctgaggaacaccgggagaagcttttaaagaaaatgcagtctttatgggaaaaagtttgtgaaaatcagagaaacctgaacgtggaaaccaccagaatcagccactggaaggattatgtgaatgtaaggctagaagctattagagctgagtatcagaagatgcctgcatttcatcatgaagaagaaaaacataatttggagatgctgaaaaagaaggggaaagatatttttcatcgacttcatttaagtaaagccaaaatggctcacaggagggagattttaagaggaacgtatgcggagctgatgaaaatgtgccataaaccagatgtggagctacttcaggcttttggagacatattacacaggagtgagtccgtgctgctgcacatgccccagcctctgaatctagagctcagggcagggcccatcactggactgagggacaggctcaaccaattccgagtggatattactctgcctcataatgaagccaacagtcatatcttccgacgtggagatttgagaagcatttgtattggatgtgaccgtcaaaatgcgccccatatcactgcaacacctacaagttttcttgcatggggtgctcagactttcacctctggcaaatattactgggaggtccatgtgggggactcttggaattgggcctttggtgtctgtaataagtattggaaagggacgaatcagaatggcaatatacatggagaggagggactctttagtcttgggtgtgttaagaatgacattcagtgcagcctctttaccacctccccacttacactgcagtatgtcccaagacctaccaaccatgtaggattattcctggattgtgaagctagaactgtgagcttcgttgatgttaatcaaagctcccctatacacaccatccctaattgctccttctcacctcctctcaggcctatcttttgctgtgttcacctctgaccagagacaaatcagatatgtgttcatctgctgtgggaacccctttatcccagaaagccctcttccttgtgccttatcaaacaggacaaatacgttctgttttatgtcttgaattgcattctaatgttattaaaactcatttattgtgttactattaaatgtggtaaaaacactaaaa

SEQ ID NO: 46 - DNA, 3438 bp, Homo sapiens
gcttttcttagtgcctaaagtgtctgcacatggaaatccagaggtagacagagagaaactgagttccggacaatgcattcactaaagaaagtgacttttgaagatgtagctattgacttcacccaggaagagtgggccatgatggacacatccaagagaaagctgtacagagatgtgatgctggaaaatatcagtcacctggtgtccctcgggtaccagataagcaaatcctatataattttgcagctggagcaaggaaaagagctgtggcgggaaggaagagaatttcttcaagaccagaatccagacagggaaagtgcccttaagaaaaaacacatgatatccatgcatcctatcaccagaaaagacgcatccaccagtatgacaatggagaactctctcattctggaggatccttttgaatgtaatgattcgggagaagattgcactcacagttccacaataactcagcgtttgttaactcacagtggaaagaaaccctatgtcagcaaacagtgtggaaaatctcttcgtaatcttttctcccctaaaccacataaacaaattcatactaaaggtaaatcatatcaatgtaatctatgtgaaaaggcctatactaattgctttcgccttagacggcacaagatgactcacactggagagaggccatatgcatgtcatctatgtggaaaagccttcactcagtgttctcaccttagaagacatgagaaaactcacacgggagagagaccatataagtgtcatcaatgtgggaaagcctttattcaatcctttaaccttcgaagacatgagagaactcaccttggaaaaaagtgttatgaatgtgataaaagtgggaaagcctttagtcaaagctctggctttagaggaaacaaaataattcacactggagagaaaccacatgcttgtcttctatgtgggaaggccttcagtctgtcttccgaccttagatgacatgagagaacatgcactggagaaaagccatatgaatgccatttatgtgggaaagccttcagtcaatgtactagtcttaaatagcatcagaaaattcacgctggagagaaaattataaacttcttcagaacatattctgactttagatgacactgtgttaggaatgacgaaggtaaggaatgtggaagagacttcagctgtagttgtagcatctaaacatgccaaaggactcatattttgaagaaatactggaatcaacatggaagatacttcagttacctttattcttcagtccacatcaataaattcatatggaagagaaattgtatgacatgtatgtaccaaagacttgttagtgatctgagcataaatgacatgagagagctgaaactgtcaacatagtcaactaaaagtcttcagcaacagctttaacttaaaacatgtgggactttcaggtagagaatctctaactctgcattcagtgtgaaaatatttttatttgcaatttattgtcaaataacatgagaaaactttacttggatgaaccctttatttgtattttctgtgaataaacattcagccaagcaccaggcttgatgttcacaagaaaacagagtgataaaatgctgctaaaatggaaaataagagaggaaagccttcataagctaaataagaagggaaagtctttccaagggcaatgaattctcttggaataccaaatacttcttactggagaagttatgcaatgaaaaatcatgagaaatcctttcttcatagagcaacacttgtggcacatgtgagatttcacactggacaaaacatggttagcatcctgaaaggagaaaattctttagtggtaattcatttcttagttgacattaagtttctcacattgaggagctatcaaacttgaaaatcactgtggagaaacctgatagatttctcatcagaaaagtgagtcaagaaggtggacctctagaaaaaactcttaacacatactttagcaaaataattcagaaatttgagaaaatatctattcataaaaatgtggcatataaatgcataatagtaaagtgaccagggaataaattgaatgcagaattatataagaaatctcattaaacttttccaaaagatcaatacttacaaatattgaaagaatacaatctgttgttgaaaaaacttagtatgttggcgaagcccttgtttcatttatgcagccctaacaaatcgatttgcatctgcactccttgggtgagattcttggtcgagattctaccccaacttctgagtctccccagtctttaacagctctttcctcacagctcacctccctttacttcaacgtccactaaaaccacttgtttccatccaaccctcgagttgacacaccagggatcttcagccccacttgctagatttctcagtgtgtcattgcatagatttagcagggaaatggaggctgtatcaaagacccctagtatatgcatttgggtgtccgcagcccttgcctctgtctctgagcagttacctgggatcagagaataaggcagctcttctcttctatccctcagaaggctcttgaaattttgtccctggagcctctctaactggaagtagcagttcatctcatgacaccccacattttattcaggtgagtcctgagttattacacagagacagacacagctgtgctccttttactgcagtccagaagataaaacaccagcatgataaaacagccgaacctgtcagccaccttgcaagcctttcctatattcgattcaatgtacttttcccgaagcaaaatgaaagttctcacagaggggccctcctctgccttgtcctcagaattggaaaatgtattgtccgtgaaggagcctcagtactgaacctaaaactcaagagaaaatgtttcctgaatatcaagtgggatgacttgaaattttgccaaacaggcacaatcttaatacgatcggccttactaaggctaaatggccttatccgtggttgaagttgacaaatcaatatattaaaaaatctccacaagtgattgattttactctgcagccagggtttatgtcaagtgtgaggataatgagcaagaaattcaagcccttggcaaactggttggagaggcaaggactgtgtccaggcagagctcatatcattatttattgtttaatctatttaattaaatatgtaatttacccacaaactgtggctgacattatatgtactcctgagccacattgagatgtactatttgtgctatgaaattctatgggatttgacaaatgcatggtggcagatctccagccattattaaagcgtaacacagaatgcttctcttattcaagctcatcttccctccacatagagggaatcaatcgacttttgtatactgatttttgaatttgcttctttatttccatcttcttcaattaaagcacaagatatcgt

SEQ ID NO: 47 - DNA, 1147 bp, Homo sapiens
gcgcgcctcgcgcggccgcggcgcgagccgggacccctggaagggtcgggtgcgcggcccaggaggtaccgctgtctcctcacaatcggccaggcctgcagccgctgccgccgctgccgccgccgccgccgccgccgccgccgccctactcagggagtggcccaggcctgcggcgcgccaccctcacctccttcattcctaccgtcaaagcccagccttggctacctgacccccctgtggtctcgaactcggttagtccggtgaagggacagagaagcctgggttcccggcggcatcactgagatgctcagtcctgaattctgccctccattaggattatctgctttgtgggataatttcctcattggttgagtcagtgagttgcgtgtctgcaagagagcatcctatctggtcggagcagagcatggacatgggaacgagtgctctcagcaagaagccgtggtggaccctgcctcaaaactttcatgcaccaatggtgtttcacatggaagaggaccaggaggagctcatcttcgggcatggtgacacataccttcgctgcattgaggtgcacagccacacccttattcagctggagagttggttcacagctacaggccagactcgtgtgactgtagtcggaccacacagggcaaggcagtggctgctgcacatgttttgttgtgtggggagccaggactcctatcatcatgctcgaggcctggagatgctggaacgtgtccgaagccagcctctgaccaacgatgacctggtcacctccattagtgtgccaccatacactggagacctgtctttggctcccaggataagtggaaccgtgtgtcttagcgttcctcagccttccccttaccaagtgattggttgttcgggtttccatttgagttcactgtatccgtaattaaggactgatctcccctgggaagtggggatgagaggagaaatcttggcttcacttttggtgagaaagacatgatagacactgaggaaagtttgaacttgtggccgggacctgtccctctcaggtcttctagacatgtctgagggaagtctcttgaatgtaagaaagtgttattggcaaagcaatccaataaaataagctacctatgatgcaaaaaaaaaaaaaaaa

SEQ ID NO: 48 - DNA, 2163 bp, Homo sapiens
ctaatcttccagttgccccctattgactttaaaccaaagctttgattcatgaccactgggatccagccagcgggggatttcttttcctgatggactgtacccaggatctcacatttgcttaagcagctcccacaattgggttcttataaaaggacacggaagagacaagctcagttttctctgaaggagaaggactgcacttagaactgcattttggcgacctctgaaattcagtactgcagtgaatgagcttctgatcttgaggtgaacttaacaaaattatttttggaaaaatcgttgtgggaaccattaaaagaactccaggaaacatgaattctggaatcttacaggtctttcagggggaactcatctgccccctgtgcatgaactacttcatagacccggtcaccatagactgtgggcacagcttttgcaggccttgtttctacctcaactggcaagacatcccatttcttgtccagtgctctgaatgcacaaagtcaaccgagcagataaacctcaaaaccaacattcatttgaagaagatggcttctcttgccagaaaagtcagtctctggctattcctgagctctgaggagcaaatgtgtggcactcacagggagacaaagaagatattctgtgaagtggacaggagcctgctctgtttgctgtgctccagctctcaggagcaccggtatcacagacaccgtcccattgagtgggctgctgaggaacaccgggagaagcttttacagaaaatgcagtctttgtgggaaaaagcttgtgaaaatcacagaaacctgaatgtggaaaccaccagaaccagatgctggaaggattatgtgaatttaaggctagaagcaattagagctgagtatcagaagatgcctgcatttcatcatgaagaagaaaaacataatttggagatgctgaaaaagaaggggaaagaaatttttcatcgacttcatttaagtaaagccaaaatggctcataggatggagattttaagaggaatgtatgaggagctgaacgaaatgtgccataaaccagatgtggagctacttcaggcttttggagacatattacacaggagtgagtccgtgctgctgcacatgccccagcctctgaatccagagctcagtgcagggcccatcactggactgagggacaggctcaaccaattccgagtgcatattactctgcatcatgaagaagccaacaatgatatctttctgtatgaaattttgagaagcatgtgtattggatgtgaccatcaagatgtaccctatttcactgcaacacctagaagttttcttgcatggggtgttcagactttcacctcgggcaaatattactgggaggtccatgtaggggactcctggaattgggcttttggtgtctgtaatatgtatcggaaagagaagaatcagaatgagaagatagatggaaaggcgggactctttcttcttgggtgtgttaagaatgacattcaatgcagtctctttaccacctccccacttatgctgcaatatatcccaaaacctaccagccgagtaggattattcctggattgtgaggctaagactgtgagctttgttgatgttaatcaaagctccctaatatacaccatccctaattgctctttctcacctcctctcaggcctatcttttgctgtattcacttctgaccagagacaaatcagaaatgtgttcacatgctgtgggaacccctttatcccaggaagtcctcttccttgtgccttaacatacaggacaaataggctctattttatgtcttgaattgccttctaatgttatcaaaactcatttattgtgttactattaaatatgctgaaaacgctaaaagtatacgtattggttctttattaaataatttttgaaaaatcattattcatgatcatggcatacagtatattctcttttttttctttatttatgactgtcactgagtgaaataatagatgacagacatgtctgaatgaagtaaaaatcaatggaagacagtcgggatcttttgcttcatgcaaaaaacttggagtgaagtctcaatgataactgggaaatgtttttcttcctctttatctaactatattacacttatccatcaggtttcattgtattaatctatcctttgaggtaata

SEQ ID NO: 49 - DNA, 437 bp, Homo sapients
caaatccatagggagctctgccttaccattgggttcctaattaactgagtgagtgggtgtgttctgcatggtgagaggcattggaatgatgcatcagaaaacatgtcataatgtcatcactgtaatatgacaagaattgcagctgtggctggaacctttataaagtgaccaagcacaccttttcatccagtctcagcgtggggtgaagcctagcagctatgaggatccattatcttctgtttgctttgctcttcctgtttttggtgcctgttccaggtcatggaggaatcataaacacattacagaaatattattgcagagtcagaggcggccggtgtgctgtgctcagctgccttccaaaggaggaacagatcggcaagtgctcgacgcgtggccgaaaatgctgccgaagaaagaaataaaaaccctgaaacatg

SEQ ID NO: 50 - DNA, 1642 bp, Homo sapiens
cctgaagctactggttggttccctgagaggtcccagaactctgcaaagtgagtccagcgctgagatttttcttgcagatccatcaggatgagcatccaggccccaccgagactactggagctggcggggcagagcctgctgagagaccaggccttgtccatctctgccatggaggagctgcccagggtgctctatctcccactcttcagggaggccttcagcaggagacacttccagactctgacggtgatggtgcaggcctggcctttcacctgcctccctctggtatcgctgatgaagacgcttcatctggagccattgaaagcattgctggaagggcttcatatgctgcttacacagaaggatcgccccaggaggtggaaacttcaagtgctggatttgcgggatgttgatgagaatttctgggccagatggcctggagcctgggccctgtcctgcttcccagaggccatgagtaagaggcagacagcagaggactgtccaaggacgggagagcaccagcccttaaaggtgttcatagacatctgcctcaaggaaataccccaggatgaatgcctgagatacctcttccagtgggtttaccaaaggagaggtttagtacacctgtgctgtagtaagctggtcaattatctaacgccaattaaatatctcagaaagtcattgaaaataatatacattaatagtattggggagctggaaattcacaacacgtgctggccacatctgataagaaagctttattgttacctgaaggagatgaagactctttgcaaactcgttttctccaggtgccatcattacacgtcagataatgaactcgagggatggttagtcaccagattcacctctgtgttcctcaggctggaacacctccagttgcttaaaataaaattgatcaccttcttcagtgggcacctggaacagctgatcaggtgcctccagaaccccttggagaacttggaattaacttgtggcaacctattagaagaggacttgaagtgtctctcccagttcccaagcctcggttacctaaagcatctgaatctcagctacgtgctgctgttccgcatcagtcttgaacccctaggagctctgctagagaaaattgctgcctctctcgagaccctcgtgttagagggctgtcagatccactactcccaactcagtgccatcctgcctggcctgagctgctgctcccagctcaccaccttctactttggcagcaattgcatgtctattgacgccctgaaggacctgctgcgccacaccagtgggctgagcaagttaagcctggagacgtatcctgcccctgaggagagtttgaattccttggttcgtgtcaattgggagatcttcaccccacttcgggctgagctgatgtgtacactgagggaattcaggcagcccaagaggatcttcattggccccaccccctgcccttcctgtggctcatcaccgtctgaggaactggagctccatctttgctgctagggaaggcgtgcccagtggggtagagaaatccaaagttctcttccaggcacttggacactaaaatctactatgtaggtgcaaactatttttctcttttcttatttatttcattttttaataattccaaaat

SEQ ID NO: 51 - DNA, 1496 bp, Homo sapiens
gccgtgtcactggctcaggctcagttgctgggtcaccagtagaatggacccttcctccacctcagttcagagcacagtgatgattcgtgactttctttcccaatagaacttcaaatctctgaagacggggggtggggggatgtgcttgagtgtttgtactcatggtcttgttctagaagtgacaaagctgggacacaatacctttatgcatgaaaaggttgtcacttgtcacaactaacaggctttcacctcacggaaattttcttcccttgtgcacttttcccctggcagtggacatggctgcactcttccaagaagcaagcagctgtcccgtctgctcagactatctagagaaaccaatgtccctggagtgtggatgcgctgtctgcttcaagtgcatcaattcactgcagaaggagccccatggggaggatctactttgctgttgctgttccatggtctctcagaagaacaaaatcaggcccagttggcagctagagaggctggcttcccacatcaaggaactggagcccaagctgaagaagattctgcagatgaacccaaggatgcggaagttccaagtggatatgaccttggatgccgacacagccaacaacttcctcctcatttctgacgacctcaggagcgtccgaagtgggtgcatcacacagaatcggcaagaccttgccgagagatttgacgtgtccatttgcatcctgggctcccctcgctttacctgtggccgccactactgggaggtggacgtgggaacaagcacagaatgggacctgggagtctgcagagaatctgttcaccgcaaagggaggatccatctgaccacagagcgtggattctggactgtgagtttgagggatggaagccgcctctctgccagcacggtgccgctgactttcctcttcgtagaccgcaagttacagcgagtggggatttttctggatatgggcatgcagaacgtttccttttttgatgctgaaggtggttcccatgtctatacattcaggagtgtctctgctgaggagccactgcacttgttttttgctcctccaagtccacctaatggtgataagagtgtcttgagtatctgtcctgtgataaacccgggcactactgatgctccagtccatcctggggaggccaaataagcccccactgcaaaaaaacaaaaaacaggttaagaaaattacttgggtgggcagacttaggaacgctctactcggtaaaagcgttatacaaagtcataggagaaaaatatgggacaattcatgattatacttaatctaatttgattagtttataaattgagtcctaagtattaattattgccaccatccaactcattgagtcttatggttcacatcttgtttcctatagaaatattctgtattctgggatcaatttccaaatgcttttttatttctgtaagttcaaattaatgtattatagaagttatgagttaaataaacattggaatatca

SEQ ID NO: 52 - DNA, 864 bp, Homo sapiens
atggctgagcacttcaaacagatcattagatgtcctgtctgtctaaaagatcttgaagaagccgtgcaactgaaatgtggatatgcctgctgcctccagtgcctcaattcactccagaaggagcccgatggggaaggtttactgtgccgtttctgctctgtggtctctcagaaggatgacatcaagcccaagtacaagctgagggcgctggtttccatcatcaaggaactagagcccaagctgaaatctgttctaacaatgaacccaaggatgaggaagtttcaagtggatatgacgttcgatgtggacacagccaacaactatctcatcatttctgaagacctgaggagtttccgaagtggggatttgagccagaataggaaggagcaagctgagaggttcgacactgccctgtgcgtcctgggcacccctcgcttcacttccggccgccattactgggaggtggacgtgggcaccagccaagtgtgggatgtgggcgtgtgcaaggaatctgtcaaccgacaggggaagattgtgctttcttcagaacacggcttcttgactgtgggttgcagagaaggaaaggtctttgctgccagcactgtgcctatgactcctctctgggtgagtccccagttgcacagagtggggattttcctggatgtaggtatgaggtccattgccttttacaatgttagtgatgggtgccatatctacacattcatcgagattcctgtttgcgagccctggcgtccattttttgctcataaacgtggaagtcaagatgatcagagcatcctgagtatctgttctgtgatcaatccatccactgccagtgccccagtttcttctgagggaaagtaa

SEQ ID NO: 53 - DNA, 594 bp, Homo sapiens
atggagacaggcaggcaaagaggtgcgtctgctgagatgttcgccatgccccaaggtctgaagggcagcaacaaggatggaatccctgaggacctagatgggaacttggaagaacccagggatcaggaaagtgagctcagaagtcaggatgtcatggacctcacagaaggtgacaatgaagcatcagcctcagctcctcctgcagccaaaagacagaaaacagataccaaggggaagaaggagaggaagcccaccgtggatgcagaggaggctcagatgacaaccctgctgtatgccatgtctgaggagcagctatcccgctacgaagtgtgtcgccggtcagctttcccaaaagcacgcattgcagctctgatgcagtctatcactggcagatcggtgtctgagaacaccgccattgccatggctggaatagctaaggtcttggttggagaggtggtggaagaggccctggacgtgtgcgagatgtggggagaaacgcccccgctgcagcccaagcatttaagggaggccgttcgcaggttaaagcccaagggcctcttccccaacagcaactataaaaaaatcatgttctaa

SEQ ID NO: 54 - DNA, 1666 bp, Homo sapiens
cctccccttgtcgcctaggtccacccgagccccctcccccgggccgcccccgagcacgaagttggcgggagcctataaaagctggtgccggcgcgacccgcggacacacagtgcaggcgcccaagccgccgccgccagatcggtgccgattcctgccctgccccgaccgccagcgcgaccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccttggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaataagatggtcccatagtctgtatccaaataatgaatcttcgggtgtttccctttagctaagcacagatctaccttggtgatttggaccctggttgctttgtgtctagttttctagacccttcatctcttacttgatagacttactaataaaatgtgaagactagaccaattgtcatgcttgacacaactgctgtggctggttggtgctttgtttatggtagtagtttttctgtaacacagaatataggataagaaataagaataaagtaccttgactttgttcacagcatgtagggtgatgagcactcacaattgttgactaaaatgctgcttttaaaacataggaaagtagaatggttgagtgcaaatccatagcacaagataaattgagctagttaaggcaaatcaggtaaaatagtcatgattctatgtaatgtaaaccagaaaaaataaatgttcatgatttcaagatgttatattaaagaaaaactttaaaaattattatatatttatagcaaagttatcttaaatatgaattctgttgtaatttaatgacttttgaattacagagatataaatgaagtattatctgtaaaaattgttataattagagttgtgatacagagtatatttccattcagacaatatatcataacttaataaatattgtattttagatatattctctaataaaattcagaattct

SEQ ID NO: 55 - DNA, 1518 bp, Homo sapiens
ttagttcttcctgaggtctgagcaccctccaaactgagtccagatctgagtttttcctcggagatttgtcagaatgagcctccaggccccatccagactgctggagctggcagggcagagcctgctgaggaaccagttcttgaccatcttcaccctggatgagctgcccagggaggtcttccctctgatgttcatggaggccttcagcatgagacgttttgaggccctgaagctgatggtgcaggcctggcccttcctcagcctccctctgggatccctgatgaagacacttcatctggagaccttgcaagctgtcctgaggggacttgatacactggtggcccagaaggttcgccccaggaggtggaaacttcaagtgctggatttgcgggatgttgatgagaatttctggaccatatggtctggagccagggtcctctcctgctccccagaggccatgagtaagaggcagacagtggaggactgtccaaggatgggagagcaccagcccttgaaggtgttcatagacctctgcctaaaggaaagtacactggatgaatgcctgagctacctttttgggtggatccactacagaagaggcctagtgcacctgtgttgtagtaaggtgcagaattactcaatgcccacttcaagtttcagaaatctattggaaaggatatacccagacagtatccaggagttggaagtctggaaaaagtgctctctcaataaaacgggaaagtttgccccttacctgagccagatgagcaatcttcgtgaactctttttagccttcggttatgagcgtgagttgtacgtgagcgtccagtggccgtgcattcctgacttggactctccattcctctgcctgtactacccccagatgctttatataaaaaagatcagtaatatcaaagagcacctggagcacctgctcaggtacctcaagaaccccttgggggcctttatattcagtgatgcttacctaactgatcgggacatggagtgtctgtctcagtacccaagcctcagtcagctaaaggagctgcgtctgattcatatcctaatgtggaccacccatcttgagccccttggagttctgctggagaaagttgctgctactctcaagaccctcgtcttaaaggactgtcggatccaggacccccaactcagggtcctcctgcctgccctgagccactgttcccagctcaccaccttcaactttcatggaaatgagacctccatgaatgctctgaaagacctgctgcgtcacacaggcgggctgagcaagttaggcctggagttgtatcctgcccctctggagagtcttgactacaagggtcatgtcaattgggagatcctcaccccaattcgggctgagctgatgcgtacactcagggaagtcaggcagcccaagaggatcttctttggtcccgtcccttgccctaactgtggctcatggccatctgagaaagtggacttccatctttgctcctagggaaggcctggttcgtggga

SEQ ID NO: 56 - DNA, 975 bp, Homo sapiens
atggctgcaaattgcaactcctcatggggtcagggaggaccctgcaacagccctgagtcagagccgccacagtctgtggcttccccagaaactcaacttggaaatcatgactgggaccctgagacttgtcacgtgaacttcaggatgttcagctgcccagaggagtcggaccccatccaggctctgaggaaactcactgagctgtgccatctgtggctgaggcccgacctccacaccaaagagcagatcctggacatgctggtgatggagcagttcatgatctccatgccccaggagctccaggtcttagtcaaggtgaacggtgtgcagagctgcaaagacctggaggacctgctacgaaataacagaagacccaagaaatggtctgtggtcaattttcttggcaaggaatatcttatgcaggaatcagatgtcgagatggctgaaatccctgccagtgtcagagatgatccgagaggcgtgtccagccagcgggcctcctctgtgaatgagatgcgtccgggggaaggccaggccagccaagagctgcagaccctgcccagggtccctgcactgtccagaaggcaggaagaggacttcctgctgccagagactactgtcatgaaaagtgccccaaaggctctgagacccaagccaaccttggagaaggatctgaaccgaatccacactggtgagaagccctacacatgtgacatctgtcacaaacggttcaacaggatgttctccttgaagtgtcacaagaggagccacacgggggagaagccctataaatgtaaggactgcaaccaagttttcacctacaggaagaacctgaacgagcacaagctcatccactccggagagaaaccctataaatgtcccaagtgtctaagagcctttcgtcggcctgaaacgttaaaataccaccagaaaacacatcaagaaaccactgcacccagagaatgtgaaggatga

SEQ ID NO: 57 - DNA, 1844 bp, Homo sapiens
gaactactgcttgattctctgagagatcccagcaccctacaaactgagtccagatctgagttttcccttgcagattcatcaagatgagcatcagggccccacccagactcctggagctggcaaggcagaggctgctgagggaccaggccttggccatctccaccatggaggagctgcccagggagctcttccccacactgttcatggaggccttcagcaggagacgctgtgaaaccctgaaaacaatggtgcaggcctggcctttcacccgcctccctctagggtccctgatgaagtcgcctcatctggagtcattaaaatctgtgctggaaggggttgatgtgctgttgacccaagaggttcgccccaggcagtcaaaacttcaagtgctggacttgaggaatgtggatgagaacttctgcgacatattttctggagctactgcatccttcccggaggctctgagtcagaagcaaacagcagataactgtccagggacaggcaggcagcagccattcatggtgttcatagacctttgtctcaagaacaggacactagatgaatgcctcacccacctcttagagtggggcaagcagagaaaaggcttactgcatgtgtgttgcaaggagctgcaggtttttggaatgcccatccacagtatcatagaggtcctgaacatggtggagcttgactgtatccaggaggtggaagtgtgctgcccctgggagctgtccactcttgtgaagtttgccccttacctgggccagatgaggaatctccgcaaacttgttctcttcaacatccgtgcatctgcctgcattcccccagacaacaaggggcagttcattgcccgattcacctctcagttcctcaagctggactatttccagaatctgtctatgcactccgtctctttcctcgaaggccacctggaccagctgctcaggtgtctccaggcctccttggagatggtcgttatgaccgactgcctgctgtcagagtcggacttgaagcatctctcttggtgcccgagcatccgtcaattaaaggagctggacctgaggggtgtcacactgacccatttcagccctgagcccctcacaggtctgctggagcaagttgtggccaccctgcagaccctggacttagaggactgtgggatcatggattcccaactcagcgccatcctgcctgtcctgagccgctgctcccagctcagcaccttcagcttctgtgggaacctcatctccatggctgcccttgagaacctgctgcgccacaccgtcgggctgagcaagctaagcctggagctgtatcctgcccctctggagagttatgacacccagggtgctctctgctgggggagatttgctgaacttggggctgagctgatgaagacactgagggacttaaggcagcccaagatcattgtgttctgcaccgtcccctgccctcgctgtggcatcagggcctcctatgacctggagcccagtcactgcctctgttgaatgcctgccatcagggtggatatatttcaagctttcttctggtcatttcggagctgaaacctaggccatgagtgcatgttaaagggagcacagacccatcgtttcaaatgcctcctcagtgtgaatgggaaaggaatgaggatgcaggaggggcaggactgggggaaaagttgacttggagtggatgggctctttagagacctgtgtcccagagaatcagaaatgggaatctgaattgctagagtgagaatcagggaggagagacacatgagagggttacccctgcacagatggttgtaaagtaacagtcagaaataaagggaaactgagtggaaa

SEQ ID NO: 58 - DNA, 1839 bp, Homo sapiens
ttcctgcttggttcttcctgaggtctgagcaccttctagactacatccagatctgttttccctgcagattcatgaagatgagcatccggattccacccagactcctggagcttgcgggcggagcctgctgagggaccaagccttggccgtctccaccctggaggagctgcccacggaacttttccccccactgttcatggaggccttcagcaggagacgctgtgaggccctgaagctgatggtgcaggcctggcccttccgccgcctccctctgaggcctctgataaagatgccttgtctggaggccttccaagctgtgctcgatgggcttgatgcactgcttacccaaggggttcgtcccaggagatggaaacttcaagtgctggatttacaggatgtctgtgagaacttctggatggtttggtctgaagctatggcccatgggtgcttcctcaatgccaagaggaacaaaacaccagtgcaggactgtccaaggatgagagaacggcagcccttgactgtgtttgtagaactttggctcaagaacaggactctggatgaatacctcacctgcctccttctatgggtcaagcagaggagagatttactacacctgtgctgtaagaagctgaaaattttgggaatgcccttccgcaatatcagaagcatcctgaaaatggtgaacctagactgtatccaggaggtggaagtgaattgcaagtggatactgcccatcctgacacagtttaccccatacctgggccacttgaggaatcttcagaagctcgttctctcccacatggatgtctctcgctacgtttccccagagcagaagaaggagattgttacccagttcaccactcagttcctcaagctgcgctgcctccaaaagctttatatgaactctgtttctttcctcgaaggccacctggaccagctgctcagctgtctgaagacctcgttaaaggtcctcacaataactaactgtgtgcttttggaatcagacttgaagcatctctcccagtgcccgagtatcagtcaactaaagaccctggacctgagtggcatcagactgaccaattacagtcttgtgcctctccaaattctcctagaaaaagttgcagccacccttgagtacctggatttagatgactgtggcatcatagactcccaagtcaacgccatcctgcctgccctgagccgctgctttgagctcaacaccttcagcttctgtggaaatcccatctgcatggccaccctggagaacctgctgagccacacaatcatactcaaaaacttatgcctggagctgtatcctgccccgcaggaaagttatggtgctgatggtactctctgctggagcagatttgctcaaattagggctgagctgatgaagaaagtgaggcacttaaggcaccccaagaggatcttgttctgtactgacaactgccctgaccatggcgacaggtcattttatgacctggaggcagatcaatactgctgttgaatgcctgcctatttggatgggtatgtcaaacgctttcttctggacacttggaaactaaaacctaggtcttaggtacatcctaaagggagcacagaacccatcgtttcacacatgggctctgaaagtgggaaaggaaagctgatcaagcaggggcaggacttgggggaaatgttgccatggattcgatgggactttgggaacctgtatcctgtagagtcgaaaatgggaatctgaatgtctagagtggaattcaggcttgagaatacatgagggagttactcttgcatggatggttgtaaagaaacaatcagaaataaaggaaaactgagcag

SEQ ID NO: 59 - DNA, 770 bp, Homo sapiens
tgctgtgaaggtattttgtagatgtgatcaacatttacaatcagttgattttaagtaaagcagtttaacttccataatgtggatgggcctcatccaattagttgaaggtgttaagagaaaagaccaaggtttcctggaaaaggaattctaccacaagactaacataaaaatgcgctgtgagtttctagcctgctggcctgccttcactgtcctgggggaggcttggagagaccaggtggactggagtagactgttgagagacgctggtctggtgaagatgtccaggaaaccacgagcctccagcccattgtccaacaaccacccaccaacaccaaagaggcgaggaagtggaaggcatcctctcaaccctggcccagaagccctatcaaagttcccaagacaacccggaagggaaaagggacccatcaaggaagttccaggaacaaaaggctctccctaaaagaccgccgcttcaaaaaaacctgaggaatggagtgggccaacactatccagccactctgaccagccgaacgaggaactcaatcaaaatgagccatagcgggaccacaagggcaaggagaccaccaccttctccagtctctcttcggacagccagtaattcccgggcaaggccagagacttcaagtctatctgaaaagtctccagaggtctaaccccagataaatagccaacagggtgtagagtacattttacaccccaaagagtgtgccccatggtgatgaaaataaagtgaacatgttgcaaaatga

SEQ ID NO: 60 - DNA, 1720 bp, Homo sapiens
agcctggagttcctgcttggttcttcctgaggactgagcaccttctagactacatccagatctgttttccctgcagattcgtgaagatgagcatccggactccacccagactcctggagcttgcagggcggagcctgctgagggaccaagccttggccatgtccaccctggaggagctgcccacagaacttttccccccactgttcatggaggccttcagcaggagacgctgtgaggccctgaagctgatggtgcaggcctggcccttccgccgcctccctctgaggcctctgataaagatgccttgtctggaggccttccaagctgtgctcgatgggctggatgcactgcttacccaaggggttcatcccaggaggtggaaacttcaagtgctggatttacaggatgtctgtgagaacttctggatggtttggtctgaagctatggcccgtgggtgcttcctcaatgccaagaggaacaaaacaccagtgcaggactgtccaaggatgagaggacagcagcccttgactgtgttcgtagaactttggctcaagaacaggactctggatgaatacctcacctgcctccttctatgggtcaagcagaggaaagatttactacacctgtgctgtaagaagctgaaaattttgggaatgcccttccgcaatatcagaagcatcctgaaaatggtgaacctagactgtatccaggaggtggaagtgaattgcaagtgggtactgcccatcctgacacagtttaccccatacctgggccacatgaggaatcttcagaagctcgttctctcccacatggatgtctctcgctacgtttccccagagcagaagaaggagattgttacccagttcaccactcagttcctcaagctgtgctgcctccaaaagctttctatgaactctgtttctttcctcgaaggccacctggaccagctgctcagctgtctgaagacctcgttaaaggtcctcacaataactaactgtgtgcttttggaatcagacttgaagcatctatcccagtgcccgagtatcagtcaactaaagaccctggacctgagtggcatcagactgaccaattacagtcttgtgcctctccaaattctcctagaaaaagttgcagccacccttgagtacctggatttagatgactgtggcatcatagactcccaagtcaacgccatcctgcctgccctgagccgctgctttgagctcaacaccttcagcttctgtggaaatcccatctccatggccaccctggagaacctgctgagccacacaatcatactcaaaaacttatgcgtggagctgtatcctgccccccgggagagttatgatgctgatggtactctctgctggagcagatttgctcaaattagggctgagctgatgaagagagtgagggacttaaggcaccccaagaggatcttgttctgtactgactgctgccctgactgtggcaacaggtcattttatgacctggaggcagatcaatgctgctgttgaatgcctgcctatttgggtggatatgtcaaacgctttcttctggacacttggaaactaaaacctaggtcttaggtacatcctatagggagcacagaacccatcatttcacacatgggctctgaaagtgggaaagggaaggtgatcaagcaggggcaggacttgggggaagtgttgccatggattcgatgggactttggggacct

SEQ ID NO: 61 - DNA, 597 bp, Homo sapiens
atggagacaggcaggcaaagagatgcctctgctgagatgttccccatgccccgaggtctgaagtacagcaacaaggatggaatccctgaggacctagatgggaacttggaagaacccagggatcaggaaagtgagctcaggagtcaggatgtcatggacctcacagaaggtgacaatgaagcgtcagcctcaactcctccttcagctaaaaggcagaaaacagataccaaggggaagaaggagaggaagcccaccatggatgcagaggaggctcagaggatgacaaccctgttttctgccatgtctgaggagcagctatctcgctacgaagtgtgtcgccggtcagctttcccaaaagcacgcattgcagctctgatgcagtccatcactggcagatcggtgtctgagaacaccgccattgccatggctggaatagccaaggtctttgtgggagaggtggtggaagaggccctggacgtgtgcgaaatgtggggagaaacgcccccgctgcagcccaagcatttacgggaggctgttcgcaggttaaagcccaagggcatcctccccaacagcaactacaaaaaaatcatgttctag

SEQ ID NO: 62 - DNA, 1642 bp, Homo sapiens
acagagtagctttgcaactggctttggggacttccgaaagctaccagcactgcactgtgagactctcatccctgagctgaattcatctgattcgacggcaagctttggtgagaacatagatatatttctgaggaaaatggactcagacttctcacatgccttccagaaggaactcacctgcgtcatctgtttgaactacctggtagaccctgtcaccatctgctgtgggcacagcttctgtaggccctgtctctgcctttcgtgggaggaagcccaaagtcctgcaaactgccctgcatgcagggaaccatcaccgaaaatggacttcaaaaccaatattcttctgaagaatttagtgaccattgccagaaaagccagtctctggcaattcctgagctctgagaaacaaatatgtgggacccataggcaaacaaagaagatgttctgtgacatggacaagagtctcctctgcttgctgtgctccaactctcaggagcacggggctcacaaacaccatcccatcgaagaggcagctgaggaacaccgggagaaactcttaaagcaaatgaggattttatggaaaaagattcaagaaaatcagagaaatctatatgaggagggaagaacagccttcctctggaggggcaatgtggttttacgggcacagatgatcaggaatgagtataggaagctgcatccggttctccataaggaagaaaaacaacatttagagagactgaacaaggaataccaagagatttttcagcaactccagagaagttgggtcaaaatggatcaaaagagtaaacacttgaaagaaatgtatcaggaactaatggaaatgtgtcataaaccagatgtggagctgctccaggatttgggagacatcgtggcaaggagtgagtccgtgctgctgcacatgccccagcctgtgaatccagagctcactgcaggacccatcactggactggtgtacaggctcaaccgcttccgagtggaaatttccttccattttgaagtaaccaatcacaatatcaggctctttgaggatgtgagaagttggatgtttagacgtggacctttgaattctgacagatctgactattttgctgcatggggagccagggtcttctcctttgggaaacactactgggagctggatgtggacaactcttgtgactgggctctgggagtctgtaacaactcctggataaggaagaatagcacaatggttaactctgaggacatatttcttcttttgtgtctgaaggtggataatcatttcaatctcttgaccacctccccagtgtttcctcactacatagagaaacctctgggccgggttggtgtgtttcttgattttgaaagtggaagtgtgagttttttgaatgtcaccaagagttccctcatatggagttacccagctggctccttaacttttcctgtcaggcctttcttttacactggccacagatgatcaggattaagaaaacttactgtttgggaactccatatacaagggagcccttcactgttgatacaaagaaatcatactgttcaggcttttttgtactttagtgtcacttcattttattgctattaaataaaaaatttgtaaaaggcaaaaaaaaaaaaaaaaaaa

SEQ ID NO: 63 - DNA, 597 bp, Homo sapiens
atggagacaggcaggcaaacaggtgtgtctgctgagatgttcgccatgccccgagatctgaagggcagcaagaaggatggaatccctgaggacctagatgggaacttggaagaacccagggatcaggaaggtgagctcaggagtgaggatgtcatggacctcacagaaggtgacaatgaggcctcagcctcagctcctcctgcagccaaaagacggaaaacagataccaaaggaaagaaggagaggaagcccactgtggatgcagaggaggctcagaggatgacaaccctgctgtctgccatgtctgaggagcagctgtcccgctacgaagtgtgtcgccggtcagctttcccaaaagcatgcattgcgggtctgatgcggtctatcactggcagatcggtgtctgagaacgtggcgattgccatggctggaatagccaaggtctttgttggagaggtggtggaagaggccctggacgtgtgtgagatgtggggagaaatgcccccactgcagcccaagcatttaagggaggctgttcgcaggttaaagcccaagggcctcttccccaacagcaactacaaaaaaatcatgttctag

SEQ ID NO: 64 - DNA, 597 bp, Homo sapiens
atggagaccggcaggcaaacaggcgtgtctgctgagatgctcgccatgccccgaggtctgaagggcagcaagaaggatggaatccctgaggacctagatgggaacttggaagaacccagggatcaggaaggtgagctcaggagtgaggatgtcatggacctcacagaaggtgacagtgaggcctcagcctcagctcctcctgcagccaaaagacggaaaacacatacgaaacgcaagaaggagaggaagcccaccgtggatgcagaggaggctcagaggatgacaaccctgctgtctgccatgtctgaggagcagctgtcccgctacgaagtgtgtcgccggtcagctttcccaagagcacgcattgcgggtctgatgcggtctatcactggcagttcggtgtctgagaacgcggccattgccatggctggaatagccaaggtctttgttggagaggtggtggaagaggccctggacatgtgtgagatgtggggagagacgcccccgctgcagcccaagcatttaagggaggctgttcacaggttaaagcccaagggcctcttccccaacagcaactacaaaagagtcatgttctag

SEQ ID NO: 65 - DNA, 1604 bp, Homo sapiens
cctccgcccgcccgcctgcctgcctgcccgcccgctcgctcgcccggcccgcgactcatgtcccgccgcaaggccggcagcgcgccccgccgagtagagcccgcgcccgccgccaacccagacgacgagatggaaatgcaggacctcgtcatcgaactcaagcccgagccagacgcgcagccccaacaggccccaaggctggggcccttctccccgaaggaggtgtcctcggcggggcggttcggcggcgaaccccaccactcccctggccccatgcccgccggggccgccctcctcgccctcggcccgcggaacccgtggaccctgtggacgccgttgaccccgaactatcccgaccgccagccctggaccgacaaacacccagatctgttgacctgcggccgctgcctgcagaccttcccgttggaggccatcactgccttcatggaccacaagaagctgggctgtcagctcttcagaggccccagccgcggccagggctcagaacgagaggagctgaaggccttgagctgcctgcgctgtggcaaacagttcacagtggcctggaagctgctgcgtcacgcccagtgggaccacggactgtccatctaccagacagaatcagaggccccggaggccccgctcctgggcctggccgaggtggctgcagccgtgtcggcagtggtggggccagcagctgaggccaagagcccccgtgcaagtggcagcggcctcacccggcggagccccacctgtcctgtgtgcaagaagaccctcagctccttcagcaacctcaaagtgcacatgcgctcacacacaggcgagcggccctatgcttgcgaccagtgtccctacgcctgcgcccagagcagcaagctcaaccgccacaagaagacccaccggcaggtgccgccccagagccccctcatggccgacaccagccaggagcaggcctctgcagcccctccggagccggctgtccatgctgctgcccccaccagcacccttccatgcagcggtggtgagggggctggagccgccgccacagcaggtgtccaggaacccggggctcctggcagtggggctcaagccggccctggtggagacacttggggagccatcaccacggaacaaagaactgaccctgcaaacagccagaaggcatcacccaaaaagatgcccaagtcagggggcaagagccgcgggcccgggggcagctgtgagttctgcgggaagcattttaccaacagcagcaacctgacggtgcaccggcgctcacacaccggggagcgcccctacacctgtgagttctgcaactacgcctgcgcccagagcagtaagctcaaccgccaccgccgcatgcacggcatgacgcctggcagcacccgcttcgagtgcccccactgccatgtgcccttcggcctgcgagccaccctggacaaacacctgcggcagaagcaccctgaggcggccggcgaggcctgagcccaggaaagcccccctcactgtccctggtaccgctgccaacacccattgacctcctcgtttttgcccgccttctccaagtaaattttcccttttatttaaaaaaaaaaaaaaaaaaa

SEQ ID NO: 66 - DNA, 4167 bp, Homo sapiens
accttcgccatatatacccggggcgctgcgctccacctggccgccgcctccagcccagcacctgcggagggagcgctgaccatggctccctggcctgaattgggagatgcccagcccaaccccgataagtacctcgaaggggccgcaggtcagcagcccactgcccctgataaaagcaaagagaccaacaaaacagataacactgaggcacctgtaaccaagattgaacttctgccgtcctactccacggctacactgatagatgagcccactgaggtggatgacccctggaacctacccactcttcaggactcggggatcaagtggtcagagagagacaccaaagggaagattctctgtttcttccaagggattgggagattgattttacttctcggatttctctactttttcgtgtgctccctggatattcttagtagcgccttccagctggttggaggaaaaatggcaggacagttcttcagcaacagctctattatgtccaaccctttgttggggctggtgatcggggtgctggtgaccgtcttggtgcagagctccagcacctcaacgtccatcgttgtcagcatggtgtcctcttcattgctcactgttcgggctgccatccccattatcatgggggccaacattggaacgtcaatcaccaacactattgttgcgctcatgcaggtgggagatcggagtgagttcagaagagcttttgcaggagccactgtccatgacttcttcaactggctgtccgtgttggtgctcttgcccgtggaggtggccacccattacctcgagatcataacccagcttatagtggagagcttccacttcaagaatggagaagatgccccagatcttctgaaagtcatcactaagcccttcacaaagctcattgtccagctggataaaaaagttatcagccaaattgcaatgaacgatgaaaaagcgaaaaacaagagtcttgtcaagatttggtgcaaaacttttaccaacaagacccagattaacgtcactgttccctcgactgctaactgcacctccccttccctctgttggacggatggcatccaaaactggaccatgaagaatgtgacctacaaggagaacatcgccaaatgccagcatatctttgtgaatttccacctcccggatcttgctgtgggcaccatcttgctcatactctccctgctggtcctctgtggttgcctgatcatgattgtcaagatcctgggctctgtgctcaaggggcaggtcgccactgtcatcaagaagaccatcaacactgatttcccctttccctttgcatggttgactggctacctggccatcctcgtcggggcaggcatgaccttcatcgtacagagcagctctgtgttcacgtcggccttgacccccctgattggaatcggcgtgataaccattgagagggcttatccactcacgctgggctccaacatcggcaccaccaccaccgccatcctggccgccttagccagccctggcaatgcattgaggagttcactccagatcgccctgtgccactttttcttcaacatctccggcatcttgctgtggtacccgatcccgttcactcgcctgcccatccgcatggccaaggggctgggcaacatctctgccaagtatcgctggttcgccgtcttctacctgatcatcttcttcttcctgatcccgctgacggtgtttggcctctcgctggccggctggcgggtgctggttggtgtcggggttcccgtcgtcttcatcatcatcctggtactgtgcctccgactcctgcagtctcgctgcccacgcgtcctgccgaagaaactccagaactggaacttcctgccgctgtggatgcgctcgctgaagccctgggatgccgtcgtctccaagttcaccggctgcttccagatgcgctgctgctgctgctgccgcgtgtgctgccgcgcgtgctgcttgctgtgtggctgccccaagtgctgccgctgcagcaagtgctgcgaggacttggaggaggcgcaggaggggcaggatgtccctgtcaaggctcctgagacctttgataacataaccattagcagagaggctcagggtgaggtccctgcctcggactcaaagaccgaatgcacggccttgtaggggacgccccagattgtcagggatggggggatggtccttgagttttgcatgctctcctccctcccacttctgcaccctttcaccacctcgaggagatttgctccccattagcgaatgaaattgatgcagtcctacctaactcgattccctttggcttggtggtaggcctgcagggcacttttattccaacccctggtcactcagtaatcttttactccaggaaggcacaggatggtacctaaagagaattagagaatgaacctggcgggacggatgtctaatcctgcgcctagctgggttggtcagtagaacctattttcagactcaaaaaccatcttcagaaagaaaaggcccagggaaggaatgtatgagaggctctcccagatgaggaagtgtactctctatgactatcaagctcaggcctctccctttttttaaaccaaagtctggcaaccaagagcagcagctccatggcctccttgccccagatcagcctgggtcaggggacatagtgtcattgtttggaaactgcagaccacaaggtgtgggtctatcccacttcctagtgctccccacattccccatcagggcttcctcacgtggacaggtgtgctagtccaggcagttcacttgcagtttccttgtcctcatgcttcggggatgggagccacgcctgaactagagttcaggctggatacatgtgctcacctgctgctcttgtcttcctaagagacagagagtggggcagatggaggagaagaaagtgaggaatgagtagcatagcattctgccaaaagggccccagattcttaatttagcaaactaagaagcccaattcaaaagcattgtggctaaagtctaacgctcctctcttggtcagataacaaaagccctccctgttggatcttttgaaataaaacgtgcaagttatccaggctcgtagcctgcatgctgccaccttgaatcccagggagtatctgcacctggaatagctctccacccctctctgcctccttactttctgtgcaagatgacttcctgggttaacttccttctttccatccacccacccactggaatctctttccaaacatttttccattttcccacagatgggctttgattagctgtcctctctccatgcctgcaaagctccagatttttggggaaagctgtacccaactggactgcccagtgaactgggatcattaagtacagtcgagcacacgtgtgtgcatgggtcaaaggggtgtgttccttctcatcctagatgccttctctgtgccttccacagcctcctgcctgattacaccactgcccccgccccaccctcagccatcccaattcttcctggccagtgcgctccagccttatctaggaaaggaggagtgggtgtagccgtgcagcaagattggggcctcccccatcccagcttctccaccatcccagcaagtcaggatatcagacagtcctcccctgaccctcccccttgtagatatcaattcccaaacagagccaaatactctatatctatagtcacagccctgtacagcatttttcataagttatatagtaaatggtctgcatgatttgtgcttctagtgctctcatttggaaatgaggcaggcttcttctatgaaatgtaaagaaagaaaccactttgtatattttgtaataccacctctgtggccatgcctgccccgcccactctgtatatatgtaagttaaacccgggcaggggctgtggccgtctttgtactctggtgatttttaaaaattgaatctttgtacttgcattgattgtataataattttgagaccaggtctcgctgtgttgctcaggctggtctcaaactcctgagatcaagcaatccgcccacctcagcctcccaaagtgctgagatcacaggcgtgagccaccaccaggcctgattgtaatttttttttttttttttttactggttatgggaagggagaaataaaatcatcaaacccaaaaaaaaaaaaaaaaaa

SEQ ID NO: 67 - DNA, 594 bp, Homo sapiens
atggagaccggcaggcaaacaggtgtgtctgctgagatgttcgccataccccgaggtctgaagggctgcaacgaggatggaatccctgaggccctagatgggaacttggaagaacccagggctcaggaatgtgagctcaggagtgaggatgtcatggacctcacagtagttgacaatgaggcctcagcctcagctcctcctgcagccaaaagacagaaaacagataccaaaggccagaaggagaggaagcccagtgtggatgcagaggaggctcagaggatgacaaccctgctgtctgccatgtctgaggagcagctgtcccgctatgaagtgtgtcgccagttagctttcccaaaagcacttgttgcacgtctgatgtggtctatcactgggagatcggtgcctgagaacatggccattgccatggctggaatagccaaggtctttgttggagaggtggtggaagaggccctggacatgtgtgagatgtggggagaaatgcccccattgcagcccatggatttaagggaggctgtttgcaggttaaagcccaagggcctcttccccaacaactataaaaaagtcatgttctag

SEQ ID NO: 68 - DNA, 1674 bp, Homo sapiens
atgtgtttcaggcacgtgctgactgtgagtggaaaaacaaaggtgactcagctgggggcaggacttggtgaaaatgctgacttggcatcaataaagccttcagggacctgtttcctagactcggaaatggaacctgaagagagacccaaagtcttcaagcctagagttcctgcttggttcttcctgaggactgagcaccttctagactacatccagatctgttttccctgcagattcgtgaagatgagcatccggactccacccagactcctggagcttgcagggcggagcctgctgagggaccaagccttggccatgtccaccctggaggagctgcccacagaacttttccccccactgttcatggaggccttcagcaggagacgctgtgaggccctgaagctgatggtgcaggcctggcccttccgccgcctccctctgaggcctctgataaagatgccttgtctggaggccttccaagctgtgctcgatgggctggatgcactgcttacccaaggggttcatcccaggaggtggaaacttcaagtgctggatttacaggatgtctgtgagaacttctggatggtttggtctgaagctatggcccatgggtgcttcctcaatgccaagaggaacaaaaaaccagtgcaggactgtccaaggatgagaggacagcagcccttgactgtgttcgtagaactttggctcaagaacaggactctggatgaatacctcacctgcctccttctatgggtcaagcagaggaaagatttactacacctgtgctgtaagaagctgaaaattttgggaatgcccttccgcaatatcagaagcatcctgaaaatggtgaacctagactgtatccaggaggtggaagtgaattgcaagtgggtactgcccatcctgacacagtttaccccatacctgggccacatgaggaatcttcagaagctcgttctctcccacatggatgtctctcgctacgtttccccagagcagaagaaggagattgttacccagttcaccactcagttcctcaagctgtgctgcctccaaaagctttctatgaactctgtttctttcctcgaaggccacctggaccagctgctcagctgtctgaagacctcgttaaaggtcctcacaataactaactgtgtgcttttggaatcagacttgaagcatctatcccagtgcccgagtatcagtcaactaaagaccctggacctgagtggcatcagactgaccaattacagtcttgtgcctctccaaattctcctagaaaaagttgcagccacccttgagtacctggatttagatgactgtggcatcatagactcccaagtcaacgccatcctgcctgccctgagctgctgctttgagctcaacaccttcagcttctgtggaaatcccatctccatggccaccctggagaacctgctgagccacacaatcatactcaaaaacttatgcgtggagctgtatcctgccccccgggagagttatgatgctgatggtactctctgctggagcagatttcctcaaattagggctgagctgatgaagagagtgagggacttaaggcaccccaagaggatcttgttctgtactgactgctgccctgactgtggcaacaggtcattttatgacctggaggcagatcaatgctgctgttga

SEQ ID NO: 69 - DNA, 1156 bp, Homo sapiens
cctggagttcctgcttggctcttcctgaggtctgagcaccttctaaactacaaccagatctgaattcttgttggcagccattttgtgaagagacgaagactgagctgttttggctgcatttctggcctcgagccgcagtcagcttctccccgtagaacccggcagtaggagacttagaatcgaatctcttctccctcccgcctcctgtttttggctttttgagaaaccttatcatccaacacaatggccagcaacgttaccaacaagatggatcctcactccgtgaactcccgtgtgttcattgggaatctcaacactcttgttgtcaagaaatctgatgtggaggcgatcttttccaagtatggcaaaattgcgggctgctctgttcataagggctttgccttcgttcaatatgataaggagaaaaatgcccgggctgctgtagcaggagaggatggcagaatgattgctagccaggttgcagttattaacctggctgcagagccaaaagtgaaccgaggaaacgcaggtgtgaaacgatccgcagcggagatgtacggctcctcttttgacttggactatggctttcaacgggattattatgatgggatgtacagtttcccagcacgtgtacctcctcctcctcccattgctctggctgtagtgccctcgaaacaccagcgcatatcaggaaacacctcacgaaggggcaaaagtggcttcaattctaagagtggaaagcggggatcttccaagtctggaaagctgaaaggagatgaccttcaggccattaagcaggagttgacccagataaaacagaaagtggattctctcctggaaaacctggaaaaaattgaaaaggaacagagcaaacaagaggtagaggtgaaaaatgctaagtcagaagaggagcagagcagtagctccatgaagaaagatgagactcatgtaaagatggagtctgaggggggtgcagaagactctgctgaggagggggacccactggatgatgatgataatgaagatcagggggacaaccagcttcatttgatcaagaataatgaaaaagatgctgaggaaggagaggataacagagacagcaccaatggccaggatgactcttaagcacatagtggggttgagaaatcttatccca

SEQ ID NO: 70 - DNA, 1353 bp, Homo sapiens
atggattcagacaccctgcgagtcttccagaatgagctcatttgctgcatttgcgtgaactacttcatagacccggtcaccactgactgtgtgcacagcttttgcaggccctgcctctgcctctgctcagaagaaggcagagcaccaatgcgctgccctttgtgcagaaaaatctcagagaagcccaacttcaacaccaatgtggcactcaaaaagctggcttccctagccagacagaccagacctcagaacatcaacagctcagacaatatctgtgtgctccatgaggagactaaggagctcttctgtgaggctgacaagagattgctctgtgggccctgctctgagtcaccagagcacatggctcacagccacagcccaataggatgggctgctgaggaatgcagggtacagaaacttataaaggaaatggactatttatggaaaatcaatcaagagacacaaaacaatctaaatcaggaaactagcaaattttgttcattagtggactatgtgtcattaaggaaggtgataatcactattcagtatcaaaagatgcatatatttctcgatgaggaggagcaacggcatctgcaggcactggaaagagaagcaaaagagcttttccaacaactacaagacagtcaagtgagaatgacccaacatttagaagggatgaaagacatgtacagagagctgtgggagacataccacatgcctgacgtggagctgctccaggatgtgggaaatatatcggcaagaactgatttggcacagatgccaaagccccagccagtgaacccagagctcacttcatggtgcataactggagtcctagacatgctcaacaacttcagagtggataatgctctgagtacagaaatgactccttgctatataagcctttctgaggatgtgagacgtgtgatatttggagatgaccatcgcagtgcacccatggatccccaaggagtggagagctttgctgtgtggtgtgcgcaagcattcacctccggcaagcattactgggaagtggatgtgacccactcctccaactggattctgggagtctgtcgagattctaggacagcagataccaatatagttattgattctgacaaaacatttttttcaatttcttcaaagacgagcaatcactatagtctctccaccaattctccacctttaatccagtatgtgcaaaggcctctgggttgggttggggtgtttctggattatgataatggatctgtgagtttttttgatgtttctaaaggttctcttatctatggttttcctccttcctccttctcttcccctctgaggcctttcttttgctttggttgtacatga

SEQ ID NO: 71 - DNA, 348 bp, Homo sapiens
atgttgctgattagtgattatgtgagtttaaggacagaagcaatcagagctgaatatcagaagatgcctgcatttctccatgaagaagagcaacatcacttggagaggctgcgaaaggagggcgaggacatttttcagcaactcaatgaaagccaagccagaatggaacatcccagggagcttttaagaggaatgtatgaggatctgaagcaaatgtaccataaagcagatgtggagctactcctggcttttggagacatattacacaggggagtgcataccaagattttagcagatgctttcagtttccacaaatatcaagcaggaactttgatattgaaggtataa

SEQ ID NO: 72 - DNA, 597 bp, Homo sapiens
atggagaccggcaggcaaacaggtgtgtctgctgagatgctcgccatgccccgaggtctgaagggcagcaagaaggatggaatccctgaggacctagacgggaacttggaagcacccagggatcaggaaggtgagctcaggagtgaggatgtcatggacctcacagaaggtgacagtgaggcctcagcctcagctcctcctgcagccaaaagacggaaaacacatacgaaaggcaagaaggagagcaagcccaccgtggatgcggaggaggctcagaggatgacaaccctgctgtctgccatgtctgaggagcagctgtcccgctacgaagtgtgtcgccggtcagcgttcccgagagcacgcgttgcgggtctgatgcgggctatcactggcagttcggtgtcggagaacgcggccattgccatggctggaatagccaagctctttgttggagaggtggtggaagaggccctggacgtgtgtgagatgtggggagagacgcccccgctgcagcccaagcatttaagggaggccgttcgcaggttaaagcccaagggcctcttccccaacagcaactgcaaaagaatcatgttctag

SEQ ID NO: 73 - DNA, 507 bp, Homo sapiens
cgccgctgcgctggtaagcctgtgttttggttcgctatggcccgtactaagcagactgctcgcaagtcgaccggcggcaaggccccgaggaagcagctggccaccaaggcggcccgcaagagcgcgccggccacgggcggggtgaagaagccgcaccgctaccggcccggcaccgtagccctgcgggagatccggcgctaccagaagtccacggagctgctgatccgcaagctgcccttccagcggctggtacgcgagatcgcgcaggactttaagacggacctgcgcttccagagctcggccgtgatggcgctgcaggaggccagcgaggcctacctggtggggctgttcgaagacacgaacctgtgcgccatccacgccaagcgcgtgaccattatgcccaaggacatccagctggcccgccgcatccgtggagagcgggcttaagaagtggcggttcggccggaggttccatcgtatccaaaaggctcttttcagagccaccca

SEQ ID NO: 74 - DNA, 1533 bp, Homo sapiens
atggattcagacgacctgcaagtcttccagaatgagctcatttgctgcatttgcgtgaactacttcatagatccggtcaccattgactgtgggcacagcttttgcaggccctgcctctgcctctgctcagaagaaggcagagcaccaatgcgctgcccttcgtgcagaaaaatctcagagaagcccaacttcaacaccaatgtggtactcaaaaagctgtcttccctagccagacagaccagacctcagaacatcaacagctcagacaatatctgtgtgctccatgaggagactaaggagctcttctgtgaggctgacaagagattgctctgtgggccctgctctgagtcaccagagcacatggctcacagccacagcccaataggatgggctgctgaggaatgcagggagaaacttataaaggaaatggactatttatgggaaatcaatcaagagacaagaaacaatctaaatcaggaaactagaacatttcattcgttaaaggactatgtgtcagtaaggaagaggataatcactattcaatatcaaaagatgcctatatttctcgatgaggaggagcaacggcatctgcaggcactggaaagagaagcagaagagcttttccaacaactacaagacagtcaagtgagaatgacccaacatttagaaaggatgaaagacatgtacagagagctgtgggagacatgccacgtgcctgacgtggagctgctccaggatgtgagaaatgtatcagcaaggactgatttggcacagatgcaaaagccccagccagtgaacccagagctcacttcatggtgcataactggagtcctagacatgctcaacaacttcagagtggatagtgctctgagcacggaaatgattccttgctatataagcctttctgaggatgtgagatatgtgatatttggagatgaccatctcagtgctcccacggatccccagggagtggacagctttgctgtgtggggagcgcaagcattcacctccggcaagcattactgggaggtggatgtgaccctctcctccaactggattctgggagtctgtcaagattccaggactgcagatgccaatttcgttattgattctgatgaaagattttttttaatttcctcaaagaggagcaatcactatagtctctccaccaactctccacctttaattcagtatgtgcaaaggcctctgggtcaagttggggtgtttctggattatgataatggatctgtgagtttttttgatgtttctaaaggttctcttatctatggttttcctccttcctccttctcttcccctctgaggcctttcttttgctttggttgtacatgaaaagttggtttcacgatgatttattgtgacctcccacatatgaggcaaatactgtcctaagacactatgtgcgagagcctgtgagctcattgtaacttcatggaatgtaattactatgtggttatgaatgggataaccaccttgaatgtgtacttttgttaattaaattattttaattaataa

SEQ ID NO: 75 - DNA, 597 bp, Homo sapiens; identical to SEQ ID NO: 72

SEQ ID NO: 76 - DNA, 597 bp, Homo sapiens
atggagaccggcaggcaaacaggcgtgtctgctgagatgctcgccatgccccgaggtctgaagggcagcaagaaggatggaatccctgaggacctagacgggaacttggaagcacccagggatcaggaaggtgagctcaggagtgaggatgtcatggacctcacagaaggtgacagtgaggcctcagcctcagctcctcctgcagccaaaagacggaaaacacatacgaaaggcaagaaggagagcaagcccaccgtggatgcggaggaggctcagaggatgacaaccctgctgtctgccatgtctgaggagcagctgtcccgctacgaagtgtgtcgccggtcagcgttcccgagagcacgcgttgcgggtctgatgcgggctatcactggcagttcggtgtcggagaacgcggccattgccatggctggaatagccaagctctttgttggagaggtggtggaagaggccctggacgtgtgtgagatgtggggagagacgcccccgctgcagcccaagcatttaagggaggccgttcgcaggttaaagcccaagggcctcttccccaacagcaactgcaaaagaatcatgttctag

SEQ ID NO: 77 - DNA, 1954 bp, Homo sapiens
atgatggcacgtcgggaccccaaatcttgggccaagagactggtgagagcccagaccctccagaagcagcggagggcccgagttgggccaagggctcccccgcccgatgaagaagatcccaggctcaagtgcaaaaactgcggggcctttggccacacggccagaagtaccaggtgccccatgaagtgctggaaggcagccctggttccagcgaccttggggaaaaaggaagggaaggaaaacctgaaaccatggaagccccgggctgaagccaacccggggcccttgaacaaggataagggagagaaggaagagagaccaaggcaacaagacccgcagaggaaggctctcctccacatgttttccgggaaacctccagagaagccgctgccaaatggaaaaggatccacggaatcttctgattatctgagggttgcaagcgggccaatgccggtccacacaaccagtaagaggccgcgcttggaccctgtcctcgctgatcgctccgcaactgaaatgtctggcaggggctccgtcttggcttcactgtctcccctcagaaaaaccagcctgagctcctcctcaagtcttggaccaaaggaaagacagactggggctgcggccgacatgcctcagcctgcagtcaggcaccagggccccgagcctcttctcgaggtgaagccgacacacagccgccccgagggtggctgccaagaagttccccaggctgcctccaaaacccacggcctgctccaggcctccagaccccaggcacaagacaaacgtcctgcggtgaccccacagccctgcccgccagccgccacacacagcttgggcctaggctccaatctcagcttcgggccaggagccaagaaacctgcccaggctccgattcaggcttgcctgaacttccccaagaaaccgagactgggtcccttccagatccccgaaagcgccatccagggaggtgagctgggggccccggagaatctccaacctccgccagccgcaaccgaacttggaccaagtacgtcgccccagatgggcaggaggacaccggcccaggtgcccagcgtcgacaggcagcctccgcacagcagaccttgcctgcccactgcccaggcctgcaccatgtcccatcactcagcggccggccatgatggggcccagcctctcagagtgctcttccggagactggaaaacggacgctggagctccagcctcctggcggccccctcatttcactctcctgagaagccgggagccttcctcgctcagagccctcatgtgtcagagaagtctgaggctccctgtgttcgtgtcccaccgagcgtcctctatgaggaccttcaggtttcctcctcctcagaggacagcgattctgacctggagtgagactgcaggtggcaggggctccttggcctccagttcccgtgacttggaggggactgtgggactgaggagcgcagagcagagagcacactctgtgcggtgactccgaagctccccggctgtggcgcttctgtggatgtgggagcccaggccaggcagggagcagatgcagggactctgcctcattgaattctggtgagggacgttgtagttggcgtggttctcccgaaacgcgccaggaaaagcttccgtgacagagattcgttgcctcagaaactgcgtgacgcgcaggagtcagacttccgctgggacgtcaataggaaactggggaattactgtgtatttgctgtctagatgactgaataagggaaaagttagggaaccctgagaggtgcagcccttccgctgtgccccgccctgagagcagtgtttcggacgctgggaagcgtgctgtgcgaagcgctctcggggtctttcctcagcctcggaaactgggctctggaatgcctttgtacatatgtgtgtttaatgtgttttgaagtgaataaaattctcaaaaaga

SEQ ID NO: 78 - DNA, 1263 bp, Homo sapiens
atggactatttatgggaaatcaatcaagagacaagaaacaatctaaatcaggaaactagcacatttcattcgttaaaggactatgtgtcagtaaggaagaggataatcactattcaatatcaaaagatgcctatatttctcgatgaggaggagcaacggcatctgcaggcactggaaagagaagcagaagagcttttccaacaactacaagacagtcaagtgagaatgacccaacatttagaaaggatgaaagacatgtacagagagctgtgggagacatgccacatgcctgacgtggtgctgctccaggtgaggagggagggtccatcctcagagacaggaagcctttgctggacagtgctgccaggacatgcaaatatcacctgcgtatgtcactgctctaagctaagtgacacatgctgtctgacttccaccattacacttctccattcacgtattactgcatactttgaaatgtatcagcaaggactgatttggcacagtatgcaaaagccccagccagtgaacccagagctcacttcatggtgcataactggagtcctagacatgctcaacaacttcagagtggatagtgctctgagcacggaaatgattccttgctatataagcctttctgaggatgtgagatatgtgatatttggagatgaccatctcagtgctcccacggatccccagggagtggacagctttgctgtgtggggagcgcaagcattcacctccggcaagcattactgggaggtggatgtgaccctctcctccaactggattctgggagtctgtcgagattccaggactgcagatgccaatttcgttattgattctgatgaaagattttttttaatttcctcaaagaggagcaatcactatagtctctccaccaactctccacctttaattcagtatgtgcaaaggcctctgggtcgagttggggtgtttctggattatgataatggatctgtgagtttttttgatgtttctaaaggttctcttatctatggttttcctccttcctccttctcttcccctctgaggcctttcttttgctttggttgtacatgaaaagttggtttcacgatgatttattgtgacctcccacatatgaggcaaatactgtcctaagacactatgtgcgagagcctgtgagctcattgtaacttcatggaatgtaattactatgtggttatgaatgggataaccaccttgaatgtgtacttttgttaattaaattattttaattaataa

SEQ ID NO: 79 - DNA, 1597 bp, Homo sapiens
acccaaagtcttcaagcctggagtttctgcttggtttttcctgaggtctgagcaccctgcaaactgagtccagatctggtaagtccctaatctctgaagatgagcatccggactccacccagactcctggagctggcggggcggagcctgctgagggacgaggccttggccatctccaccctggaggagctgcccacggaactttttcccccattgttcatggaggccttcagcaggagacactgtgaggccctgaagctgatggtgcaggcctggcctttcctccgccttcctctggggtctctgatgaaaaggccttgcccagagaccttccaagctgtgctcgatgggcttgatgcactgcttacccacagggttcgtctcaggaggtggaaacttcaagtgctggatttacaggatgtcagtgagaacttctggatggtttggtctgaagccatggcccgtaggtgcttaccaaatgccatgatgaacagaaaaccagtgcaggactgtccaaggatgagaggacagcagcccttgactgtgttcatagacctttgcctcaagaacaggactctggatgaatacttcacctgcctctttctatgggtcaagcagagggaaggtttagtacacctgtgctgtaagaagctgaaaatgttgggaatgctcttccacaatatcagaaacatcctgaaaacagtcaacctagactgtatccaggaggtggaagtgaattgcaattggacactgcccgtcctggcagagtttaccccatacctcggccagatgaggaatcttcggaagctcgttctctctgacatagattctcgctacatttccccagagcagaagaaggagtttgttacccagttcaccactcagttcctcaagctgcgctgcctccaaaagctttatatgaactctgtttctttcctcgaaggccacctggaccagatgctcagctgtctaaagacctcgttaaacatcctcgcaataactaactgtgtgcttttggaatcagacttgaagcatctgtccaagtacccgagcattggtcaactaaagaccctggacctgagtggcaccagactggccaatttcagccttgtgccgctccaagttctcctagaaaaagttgcagccacccttgagtacctggacttagatgactgtggcatcgtagactcccaagtcaacgccatcctgcctgccctgagccgctgctttgagctcaccaccttcagcttccgtggaaatcccatctccacggccaccctggagaacctgctgtgccacacaatcagactcaacaacttatgcctggagctgtatcctgccccgcgggagagttatgatgttcgtggtatcgtctgccggagcagatttgcccaacttggggctgagctgatggggagagtgagggccttaagggagcccgagaggatcttgttctgtaccgactactgccctcagtgtggcaacaggtcactttacgacctggaggtagatcggtgttgctgttgaatgcctgcctatttgggtggatatatcaaactcttttttctgaacacttgaaaactaaaacctaggtctt

SEQ ID NO: 80 - DNA, 576 bp, Homo sapiens
actcttctggtccccacagactcagagagaacccaccatggtgctgtctcctgccgacaagaccaacgtcaaggccgcctggggtaaggtcggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtggccatgcttcttgccccttgggcctccccccagcccctcctccccttcctgcacccgtacccccgtggtctttgaataaagtctgagtgggcggc

SEQ ID NO: 81 - DNA, 1674 bp, Homo sapiens
cttagagggagctgtattttggtgacctctaaaagtcagtactgcggtgaatgagctccagaacttgaggagtacttaacagaattatttctggaagaatcactgtgggaaccattcaaagaacccagcgaaacatgaattctggaatctcgcaagacttccagatggaaattacctgccccatctgcatgaattacttcatagacccagtcaccatagactgtgggcacagcttttgcaggccctgtttctacttcaactggcaagacatcccaattcttactcagtgctttgaatgcatgaagacaacatggcagagaaacctgaaaactaacattcatttgaagcagatggcttcccttgccagaaaagccagtctctggctattcctgagctctgaggagcaaatgtgtggcactcacagggagacaaagaagatattctgtgaagtggacaggagcctgctctgtttgctgtgctccagctctcaggagcaccggtatcacagacaccgtcccattgagtgggctgctgaggaacaccgggagaagcttttaaagaaaatgcagtctttatgggaaaaagcttgtgaaaatcagagaaacctgaatgtggaaaccaccagaatcagccactggaaggattatgtgaatttaaggctagaagctatgagagctgagtatcagaagatggctgcatttcaccatgaagaagaaaaacataatttggagatgctgaaaaagaaggggaaagatatttttcatcaacttcatttaagtaaagccaaaatggctcataggagggagattttaagaggaatgtatgaggagctgaaggaaatgtgccataaaccagatgtggagctacttcagggtttcggagacatattacacaggagtgagtccgtgctgctgcacatgccccagcctctgaatctagagctcagtgcagggcccatcactggactgagggacaggctcatccaattctgagtggatattactctgcctcataatgaagccaacagtcatatcttccgatgtggagatttgagaagcatgtgtattggatgtgaccgtcaaaatgtgccccatatcactgcaacacctacaagttttcttgcatggggtgctcagactttcacctctggcaaatattactgggaggtccatgtgggggactcttggaattgggcctttggtgtctgtaataagtattggaaagggaagaatcagaatgacaatatatatggagaggagggactctttagtcttggatgtgtcaagaatgacattcagtgcagtctctttaccacctccccacttacactgcaatatatcccaagaactaccagccgcataggattattcctggattgtgaagctagaactgagcttcgttgatgttaaccaaagctgccttatatacaccatccctaattgctccttctcacctcctctcaggcctatctttcggtgtattcacctctgaccagagataaatcagaaatgtgttcatctgctgtgagaatccctttattccaggaagccctcttccttgtgccttatcaaacaggacaaataggttctgttttatgtcttgaattgcctcctaatgttattaaaactcatttattgtgttactattaaaaatggtaaaaacactaaaagtata

SEQ ID NO: 82 - DNA, 1630 bp, Homo sapiens
gcaaggaatgagctcctgatcttggggagtacttaaaagaactttttcttggaagaattactgcaggaaacattcatagaaccttgggaaacatgaattctggaatcttgcaagtcttccagagggcactcacctgtcccatctgcatgaactacttcatagacccagtcaccatagactgtgggcacaacttttgccggccctgtttctacctcaactggcgagacatggcagttcttgctcagtgctctaaatgcaagaagacaatacagcagagaaacctcaaaactgacatttgtttgaagaacatggcttccactgccagaaaagccagcctctggcaattccttagctctgaggagcaaatatgtgggatgcacagagagacaaagaagatgttctgtgaagtgaacaagagccggctctgttggctgtgctccaactctcaggagcaccggaatcacagacactgtcccattgagtgggctgctgaggaacgccgggaggagctcctaaaaaaaatgcagtctttatggcaaaaagcttgtgaaaatctcagaaacctgaacacagaaaccaccagaaccagatgctggaaggattatgtgagtttaaggacagaagcaatcagagctgaatatcagaagatgcctgcatttctccatgaagaagagcaacatcacttggagaggctgcgaaaggagggcgaggacatttttcagcaactcaatgaaagccaagccagaatggaacatcccagggagcttttaagaggaatgtatgaggatctgaagcaaatgtaccataaagcagatgtggagctactcctggcttttggagacatattacacaggggagtgtatgagtccccgctgctgcaagtgtctgagcctgtgaatccagagctcagtgcagggcccatcactggactgctggacaggctcagtggattcagagttgattttactctgcagcctgaaagaaccaatagtcatatcttcctgtatggagatttgagaagcatgaatgttggatgtgaccctcaagatgatcccgatatcactgcaaaatctgaatgttttcttgtagggggggctccggctttcacatctggcaaatattattgggaggttcacgtgggggactcttggaattgggcttttggtgtctgtaacaattatcggaaagagaagagacagaatgacaagatagatggagaggagggactctttcttcttggatgtgttaaggaggacactcactgcagtctctttaccacctccccacttgtggtgcaatatgttccaagacctaccagcacagtaggattattcctggattgtgaaggtagaaccatgagctttgttggtgttgatcaaagttccctgatatacaccatccctaattgctccttctcacctcctctcaggcctatcttttgctgtagtcacttctgaccagagaaaagtcagaaatgtgtctatatgctctgggaacctgtttatcccagaaagccctctttttcgcacctcatcaaatgggacaaataagttacatttaatgtctttagttgcattctaatgtcatcaaaactcatttatagtgtttctattaaatatggtgaaaacac

SEQ ID NO: 83 - DNA, 622 bp, Homo sapiens
cataaaccctggcgcgctcgcgggccggcactcttctggtccccacagactcagagagaacccaccatggtgctgtctcctgccgacaagaccaacgtcaaggccgcctggggtaaggtcggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtagccgttcctcctgcccgctgggcctcccaacgggccctcctcccctccttgcaccggcccttcctggtctttgaataaagtctgagtgggcagcaaaaaaaaaaaaaaaaaa

SEQ ID NO: 84 - DNA, 597 bp, Homo sapiens
atggagaccggcaggcaaacaggtgtgtctgctgagatgctcgccatgccccgaggtctgaagggcagcaagaaggatggaatccctgaggacctagacgggaacttggaagcacccagggatcaggaaggtgagctcaggagtgaggatgtcatggacctcacagaaggtgacagtgaggcctcagcctcagctcctcctgcagccaaaagacggaaaacacatacgaaaggcaagaaggagagcaagcccaccgtggatgcggaggaggctcagaggatgacaaccctgctgtctgccatgtctgaggagcagctgtcccgctacgaagtgtgtcgccggtcagcgttcccgagagcacgcgttgcgggtctgatgcgggctatcactggcagttcggtgtcggagaacgcggccattgccatggctggaatagccaagctttttgttggagaggtggtggaagaggccctggacgtgtgtgagatgtggggagagacgctcccgctgcagcccaagcatttaagggaggccgttcgcaggttaaagcccaagggcctcttccccaacagcaactgcaaaagaatcatgttctag

SEQ ID NO: 85 - DNA, 1369 bp, Homo sapiens
atgaattctggaatctcgcaagtattccagagggcactcacctgtcccatctacatgaactacttcctagacccagtcaccatagactgtgggcacagcttttgccggtcctgtttgtacctcaactggcaagacatggcagttcctgcccagtgctctaaatgcaagaagacaacacggcagagaaacctcaacactgacatttgtttgaagaacatggcttccattgccagaaaagccagcctccggcaattccttagctctgaggagcaaatatgtgggatgcacagacacaaaaagaaggtgttctgtgaagtggacaagagcctgctctgttttctgtgctccaactctcaggagcaccgagatcacagacactgtcccattgagttggctgctgaggaataccgggaggagctcctaaaaaaaaatgcagtgcttatgggaaaaagcttgtgaaaatctcagaaaccagaacatggaaaccaccagaaccagatgctggaagtttagtaccgattatgtgagtttaaggatagaagcaatcagagctgaatatcagaagatgcttgcatttctccgtgaagaagagcaacatcacttggagaggctgcgaaaggagggcgaggacatttttcagcaactcaatgaaagcaaagccagaatggaacattccagggagcttttaagaggaatgtatgaggatctgaagcaaatgtgccataaagcagatgtggagctactccagtcttttggaaacatattacaaaggtatgagtccctgctgctgcaagtgtctgagcctgtgaatccagagctcagtgcagggcccatcactggactgctggacaggctcagtggattcagagttgattttactctgcggcttgaaagagctaatagtcatatcttcctgtatggagatttgagaagcatgaatgttggatgtgaccctcaagatgatcccgatatcactgcaaaatctgaatgttttcttgtatgggggactcaggctttcacatctggcaaatattattgggaggtgcacgtgggggattcttggaattgggcttttggtgtctgtaacaattattggaaagagaagagacagaatgacaagatagatggagaggagggactctttcttcttggatgtgttaaggaggacactcactgcagtctctttaccaccaccccacttgtggtgcaatatgttccaaaacctaccagcacagtaggattattcctggattgtgaaggtagaatcatgagctttgttgatgttgatcaaagtttcctgatatacatcatccctaattgctccttctcacctcctctcaggcctatcttttgctgtagtcacttctga

SEQ ID NO: 86 - DNA, 624 bp, Homo sapiens
atgggaacaaacttgtgtttgggtgaaacaggcttccccattgcagttactataacacctgtgtggtacctcaagaaccccttgggggcctttatattcagtgatgcttacctaactgatcgggacatggagtgtctgtctcagtacccaagcctcagtcagctaaaggagctgcgtctgattcatatcctaatgtggaccaccaatcttgagccccttggagttctgctggagaaagttgctgctactctcaagaccctcgtcttaaaggactgtcggatccaggacccccaactcagggtcctcctgcctgccctgagccactgttcccagctcaccaccttcaactttcatggaaatgagacctccatgaatgctctgaaagacctgctgcgtcacacacgtgggctgagcaagttaggcctggagttgtatcctgcccctctggagagtcttgactacaagggtcatgtcaattgggagatcctcaccccaattcgggctgagctgatgcgtacactcagggaagtcaggcagcccaagaggatcttctttggtcccgtcccttgccctacctgtggctcatggccatctgagaaagtggacttccatctttgctcctag

SEQ ID NO: 87 - DNA, 909 bp, Homo sapiens
atgcattcactaaagaaagtgacttttgaagatgtagctattgacttcacccaggaagagtgagccatgatggacacatccaagagaaagctgtacagagatgtgatgctggaaaatatcagtcacctggtgtccctcgtaacagggtaccagataagcaaatcctatataattttgcagctggagcaaggaaaagagctgtggcaggaaggaagagaatttcttcaagaccagaatccagacagggaaagtgcccttaagaaaacacacttgatatccatgcatcctatcatcagaaaagatgcacccaccagtatgacaatggagaactctctcattctggaggatccttttgaatgtaatgattcgggagaagattgcactcacagttccacaataattcagtgtttgttaactcatagtggaaagaaaccctatgtcagcaaacagtgtggaaaatcccttagtaatcttttgtcccctaaaccacataaacaaattcatactaaaggtaaatcatatcaatgtaatctatgtgaaaaggcctatactaattgctttcaccttagacggcccaagatgactcacactggagagaggccatatacatgtcatctatgtagaaaagccttcactcagtgttctcaccttagaagacatgagaaaactcacacaggagagagaccatataagtgtcatcaatgtgggaaagcctttattcaatcctttaaccttcgaagacatgagagaactcaccttggagaaaagtggtatgaatgtgataacagtgggaaagcctttagtcaaagctctggctttagaggaaacaaaataattcacactggagagaaaccacatgcttgtcttctatgtgggaaggccttcagtctgtcttccgaccttagatga

SEQ ID NO: 88 - DNA, 1705 bp, Homo sapiens
aagtttgagtttgtgtgcttactagcaaccactttcagcgtttctgcaaagctagagaagggttcccagtgccgattccgacactgtgaagaggcccttggcagtgacactgtgtgctcattatggagagagaggaaatgtttagatccactttgcagatttagacacatggaaatgcagcaaaactgcagcatttcatgcttctgggaaactcagcctcttggttgtgtgaagatcagctgtatcttttatcacagcaaacctcgaaatatcaatggattatttttgccaccaagtagcaatatcacactacagaaagaaattcaggaaggaattccactccagtcccagagtcaagaacctctgaaacctcaggaaaatatatcacgacccatccaccatcctttagttttaaaaactaattttgaggaagaagaggaagtagatgaacaaaatgatgcttctagtttatggacaaagacccctgaagaaattgaagaaaaaagagcaataaaggagatgtgttataaatctggggaatactacagatttcatactcctccagatattttgtcatcaaaaagcatgacacctacagcagaaaaacagttagaaaagcctttggaaaatggcagtgaattgcaagaaggagatagtcttacagttccgacaaaacttagccaatatgaaaggcaaggtgaaataaaaacatcattgcatgggaaaccaaagactgacattgctgcttttgaaaatggaggaggtgactgttatgttccacagagggtcatatttcttggagtcgatgaaagtgaagctttaactgaagagaaagaaatcaccatatcaaaatgttcaaatactaaagataacaaggacagtcctcatccaaagcattccctaactacccgactagtacctacaacgcatgtattaaatgctactgagaatatcagtatgaagtgcagagaggacccctcttcaatgaatgatgtccagccagtgaagaagcctcattttaaaggtgtgaagaaaagaaaatggatttatgatgaaccacagaactttcctaactctggaatgcagagagcagtacaggccccaagaccccaaaataaaatgagttatcaccgcaataataaaaatcgaaatgcggagaatgcatcctatatccacgttcaaagagatgctgtcaggactgtcgcgttgaatgcaccttcccgcagcaggcccacgcatgggtcctacaataaagtccatgctaacagggaacccaaacccaacctcagtccagacaaatatacgtcaacatcatataatgattcagcctggcgaaaacgaattcctttttcaaaaacatattcaaaaagtgaaaagatatatccagagccaagaagaaatgggagcaagtaaacctggaggttgagagagagaaaaatgagaaaaaattgccagacatcatctgatttacaatcaaaaatgaaaattcaaagaatgagatactcaagtactccatgttatgtagtggatagccaaagatgataaaaatgcaaattctgaaaataagatgcaggaagtacattgtgctctttcaaaactgataaaaatgcaaattaccaacagctgccagtgtatttatgtaggtctggaattcattccgtgcattactccaaacattttaataaaatttaaaagtttgacaatt

SEQ ID NO: 89 - DNA, 957 bp, Homo sapiens
atgtgctcagggaatcagacttctcagaatcaaacagcaagcactgatttcaccctcacgggactctttgctgagagcaagcatgctgccctcctctacaccgtgaccttccttcttttcttgatggccctcactgggaatgccctcctcatcctcctcatccactcagagccccgcctccacacccccatgtacttcttcatcagccagctcgcgctcatggatctcatgtacctatgcgtgactgtgcccaagatgcttgtgggccaggtcactggagatgataccatttccccgtcaggctgtgggatccagatgttcttccacctgaccctggctggagctgaggttttcctcctggctgccatggcctatgaccgatatgctgctgtttgcagacctctccattacccactgctgatgaaccagagggtgtgccagctcctggtgtcagcctgctgggttttgggaatggttgatggtttgttgctcacccccattaccatgagcttccccttttgccagtctaggaaaatcctgagttttttctgtgagactcctgccctgctgaagctctcctgctctgacgtctccctctataagatgctcacgtacctgtgctgcatcctcatgcttctcacccccatcatggtcatctccagctcatacaccctcatcctgcatctcatccacaggatgaattctgccgccggccgcaggaaggccttggccacctgctcctcccacatgatcatagtgctgctgctcttcggtgcttccttctacacctacatgctccggagttcctaccacacagctgagcaggacatgatggtgtctgccttttacaccatcttcactcctgtgctgaaccccctcatttacagtctccgcaacaaagatgtcaccagggctctgaggagcatgatgcagtcaagaatgaaccaagaaaagtag

SEQ ID NO: 90 - DNA, 1839 bp, Homo sapiens
agaaagtgctagaattttactttcagaaggaccagtacccgaactacgaccagcgactgaatctggcggagatgctcagcctcagggagcaacagctgcaggtgtggttcaagaatcgccgcgccaaactagctcgggagcggcggctccagcagcagccccagcgcgtccctgggcagagaggccgaggagcccgcgctgcgcccctagtccctgcagcctctgcctccgcacctcagcggggcccctcgggaatccttccagcggcggaacccacgatctgcagcctccaccaggcctggggtggccctgggtgcagagcccagaagggcatcccagctgccctgagtccaggccctggcccgatccctgccccaatcccaggcccagcccagatcccaggcccactccctggctcaattccaggcccaattccaggcccagctcagatcccaagcccgatcccagccccaatcccaggcccaatttcaggcccagtccagatcccaggcccattccgtggcccaatcccaggcccaatttcaggcccagccccgatcccaggcccaatctcaggcccattctcaggcccaaacccaggcccgatcccaggcccaaacccaggcccgatcccaggcccaatctcaggcccgatcccaggcccaatctcagtcccgatcccaggcccgatcccaggcccaatctcaggcccaatctcaggcccaaacccaggcccgatcccaggcccaatcccaggcccaatctcaggcccgaacccaggcccgatcccaggcccaatctcaggcccgaacccaggcctgatcccaggcccaatcccaggcccaatctcaggcccaggcccaattataggcccgattcccagcccagcccagatcccaggcccaggcagactccaaggcccaggtcccatcttaagtcctggccggatgcgaagccctggctcacttccaggcctagccccgattttaggcccaggctcaggcccaggctcaggctcagtcccagctccaatcccaggcccaggatcactcccagccccagcccccttatggcctcagagccccgatgcctccgacttcttgccagacacccagttattccctcacttcacagagctgctcctacccctagaccccttggagggatcctcagtctccaccatgacctctcagtaccaagaaggggatgactctatgggcaaaaaacactcagggtctcagccccaagaggagggtggctctgtgaatgaaaatcactcaggccccaggttattactggatttatagggggcctgtgcctgcaaagttcttcagatcccagagggcctggagtggctgatctacactgctggtgactttctgcagtgaatgcatcaccctttacccaccctgctgcaggtggacatttgctgtcttcactgtgcagctatcacaggtggctctgctgtgaatgtgcctgcctgtcatttgaattgagcctgagcgtttctctgctgggattggagtcgctgctcactgggatatctggcattagtggatgctgctcgagagctttctaaagggcttttcccagggcttctgatggtcagcatcttccccaacacttaggattgtgagactttttaatgtttgtcgatccaatgacttgtggttttgttgtggttgtaatatccatttccctgatgattgagaagttaaaccctcacatgttaattgaaccttagatatcttcataatgtgtatttgtttaccccttttccttcatttatttgaatgtattcttatattctcttggcaattaaagttctgcagacacctt

SEQ ID NO: 91 - DNA, 597 bp, Homo sapiens
atggagaccggcaggcaaacaggcgtgtctgctgagatgctcgccatgccccgaggtctgaagggcagcaagaaggatggaatccctgaggacctagacgggaacttggaagcacccagggatcaggaaggtgagctcaggagtgaggatgtcatggacctcacagaaggtgacagtgaggcctcagcctcagctcctcctgcagccaaaagacggaaaacacatacgaaaggcaagaaggagagcaagcccaccgtggatgcggaggaggctcagaggatgacaaccctgctgtctgccatgtctgaggagcagctgtcccgctacgaagtgtgtcgccggtcagcgttcccgagagcacgcgttgcgggtctgatgcgggctatcactggcagttcggtgtcggagaacgcggccattgccatggctggaatagccaagctctttgttggagaggtggtggaagaggccctggacgtgtgtgagatgtggggagagacgcccccgctgcagcccaagcatttaagggaggccgttcgcaggttaaagcccaagggcctcttccccaacagcaactgcagaagaatcatgttctag

SEQ ID NO: 92 - DNA, 597 bp, Homo sapiens; identical to SEQ ID NO: 72

SEQ ID NO: 93 - DNA, 1565 bp, Homo sapiens
ctcactgtcaacaacatcccatcctgaagacttgcttaccacaactgagagtggggagtcagtttgaggcctagcatcacttaaacttcctccaaaggcacaaaagccagaagagaaaaatgaagacgggacattttgaaatagtcaccatgctgctggcaaccatgattctagtggacattttccaggtgaaggctgaagtgttagacatggcagataatgcatttgatgatgaatacctgaaatgtacggacaggatggaaattaaatacgttccccaactgctaaaggaggaaaaagcaagccaccagcaattagatactgtgtgggaaaatgcaaaagccaaatgggcagcccgaaagactcaaatctttctccctatgaattttaaggataaccatggaatagccctgatggcatatatttccgaagctcaagagcaaactcccttttaccatctgttcagtgaagctgtgaagatggctggccaatctcgagaagattatatctatggcttccagttcaaagctttccacttttacctcacaagagccctgcagttgctgagaaaaccttgtgaggccagttccaaaactgtggtatatagaacaagccagggcacttcatttacatttggagggctaaaccaagccaggtttggccattttaccttggcatattcagccaaacctcaggctgctaatgaccagctcactgtgttatccatctacacatgccttggagttgacattgaaaattttcttgataaagaaagtgaaagaattactttaatacctctgaatgaggtttttcaagtgtcacaggagggggctggcaataaccttatccttcaaagcataaacaagacctgcagccattatgagtgtgcatttctaggtggactaaaaaccgaaaactgtattgagaacctagaatattttcaacccatctatgtctacaaccctggtgagaaaaaccagaagcttgaagaccatagtgagaaaaactggaagcttgaagaccatggtgagaaaaaccagaagcttgaagaccatggtgtgaaaatccttgaacccacccaaatacctgaagataaaagtcaaggaaatatcaacaatcctactccaggtccagttcctgttccaggtcccaaaagccatccttctgcatcctcgggcaaactgctgcttccacagtttgggatggtcatcattttaatcagtgtttctgctataaatctctttgttgctctgtagtttgatgcattgtttatctttcttattctttacttgaaataactatagggatccacaggagatcaaaaggaatgatgtattttttacgtgttggccaaagtcactggataaaatgagaattgtatatttgttattcattttgcaaattcagaaagttggtccagatatatgtcacagaacttttcacttgtatactactcttacaatggaaaaaaatcccgaaaactgtatacttctgattaaattcaataaaagattttgattagatatttcagaattgccaaaaaaaaaaaaaaaaaaaaaaaaaaa

SEQ ID NO: 94 - DNA, 908 bp, Homo sapiens
agctggaggccaggggagaaactccagaaggagagacatttgccatggctgagcacttcaaacagatcattagatgtcctgtctgtctaaaagatcttgaagaagccgtgcaactgaaatgtggatatgcctgctgcctccagtgcctcaattcactccagaaggagcccgatggggaaggtttactgtgccgtttctgctctgtggtctctcagaaggatgacatcaagcccaagtacaagctgagggcgctggtttccatcatcaaggaactagagcccaagctgaaatctgttctaacaatgaacccaaggatgaggaagtttcaagtggatatgacgttcgatgtggacacagccaacaactatctcatcatttctgaagacctgaggagtttccgaagtggggatttgagccagaataggaaggagcaagctgagaggttcgacactgccctgtgcgtcctgggcacccctcgcttcacttccggccgccattactgggaggtggacgtgggcaccagccaagtgtgggatgtgggcgtgtgcaaggaatctgtgaaccgacaggggaagattgtgctttcttcagaacacggcttcttgactgtgggttgcagagaaggaaaggtctttgctgccagcactgtgcctatgactcctctctgggtgagtccccagttgcacagagtggggattttcctggatgtaggtatgaggtccattgccttttacaatgttagtgatgggtgccatatctacacattcatcgagattcctgtttgcgagccctggcgtccattttttgctcataaacgtggaagtcaagatgatcagagcatcctgagtatctgttctgtgatcaatccatccgctgccagtgccccagtttcttctgagggaaagtaa

SEQ ID NO: 95 - DNA, 1038 bp, Homo sapiens
atgaatttggagggcacttcaggtggcatacttcaaaaagaattctggagaaacagaattcagtataaccagagtcaaaaggatatcctccaatcatggtttcaacatgaccctttccctgataaagctgccagagaacaactggccaaagaaattggggttccagaatctaatattcaggtttggtttaaaaattacagagtaaaacagagaaaactggattataagtgcttctcagaaaaagatcaaacccaggggcatgaccagtcccagcatctgactcaagaatacttacctaaagaagcccgacaaaaacagacattcatcacatggactcaaaaaaacaggctagtgcaagcctttgagaggaacccattccctgatattgctaccagaaaaaaactggctgaacaaacaggcctgcaggaatcaagaattcaaatgtggtttcagaaacaaagatctctgtacctcaagaagagcagaatggagcccatgaatttattggtagacgacccaaatgagagaccagatgcaactgttgggtggcatccaatcaacctgttcctccccacagacagctctcattatttttcttgctcacattcttccagcgggcatgaaactcttccacctgttcttccttcaacccaggctccttgggatcccttcaggttccatgtgagccaaggaccaaatgtcatgatcatgcagcccacacaggctgtgcaggaaggagagaagtctgatcagcctctgataattccgaatcacctcctgacactgccaattctgacaaaggacttagatactccgactcccttctggctccaataccaagaagaacaccaaaatcacaaagaacactctggctcgggagtaccacaggtcaagagccattctcagcctgaacctgagcacagggagcaacaacctctaaatctgggtcagtttgacatatcgaacattttgcaaaggtgggacgagatctgccaggctctgcttgctgaatgggaccctctcaaagggacacactga

SEQ ID NO: 96 - DNA, 2346 bp, Homo sapiens
cggcgcgcgacgtgctttgctgtataaatgcggtggcgcccggcgtagggacacttcggtcctgagcgcttgggagttaggttgtttgccggcgtagcggccagcgcctgagcccgcccttgatcttcgctgtggcatggcggacgaggggaagtcgtacagcgaacacgatgatgaacgcgttaatttccctcaaagaaagaagaaaggccggggtcccttccggtggaaatatggtgaaggaaaccgtaggtctggaagaggcggttctggtattcggtcttcccgccttgaggaagatgatggagatgtggcaatgagtgatgcccaggatggtccccgagtacgatacaacccctataccacccgacctaaccgtcggggtgatacttggcatgatcgagatcgcattcatgttactgtgcggagagacagagctcctccagagagaggaggggctggcaccagccaggatgggacctcaaagaactggttcaagattacaattccttatggcagaaagtatgacaaggcatggctcctgagcatgattcagagcaagtgcagtgtgcccttcacccctattgagtttcactatgagaatacacgggcccagttcttcgttgaagacgccagtactgcctctgcattgaaggctgtcaactataagattttggatcgggagaaccgaaggatatctatcatcatcaactcttctgctccaccccacactatactgaatgaactgaagccagaacaagtagaacagctaaagctgatcatgagcaaacgatacgatggctcccaacaagcccttgacctcaaaggcctccgttcagacccagatttggtggcccagaacattgacgttgtcctgaatcgcagaagctgtatggcagctaccctgaggatcattgaagagaacatccctgagctattgtccttgaacttgagcaacaacaggctgtacaggctggatgacatgtctagcattgttcagaaggcacccaacctgaagatcctaaacctttctggaaatgaattgaagtctgagcgggaattggacaagataaaggggctgaagctagaagagctctggctcgatggaaactccctgtgtgacaccttccgagaccagtccacctacatcagcgccattcgcgaacgatttcccaagttactacgcctggatggccatgagctacccccaccaattgcctttgatgttgaagcccccacgacgttaccgccctgcaagggaagctattttggaacagaaaacttgaagagtctggtcttgcacttcctgcaacagtactatgcaatttacgactctggagaccgacaagggctcctggatgcctaccatgatggggcctgctgttccctgagcattcctttcattcctcagaaccctgcccgaagcagcttagccgagtatttcaaggatagcagaaatgtgaagaagcttaaagaccctaccttgcggttccggctgctgaagcacacgcgtctcaacgttgttgccttcctcaatgagttgcccaaaacccagcacgacgtcaattccttcgtggtagacataagcgcccagacaagcacattgctgtgtttttctgtcaatggagtcttcaaggaagtggacggaaagtcccgggattctttgcgagccttcacccggacattcattgctgttcctgctagcaattcagggctatgtattgtaaatgatgagctatttgtgcggaatgccagttctgaagagatccaaagagccttcgctatgcctgcacccacgccttcctccagcccggtgcccaccctctctccagagcagcaggaaatgttgcaagcattctctacccagtctggcatgaacctcgagtggtcccagaagtgccttcaggacaacaactgggactacaccagatctgcccaggccttcactcatctcaaggccaagggcgagatcccagaagtggcattcatgaagtgatcgtagtcatgcctcagaagcagtcccccctgtaaatagtccttggatattaccgtctggttgtcgtctgtcatctcctcctgtctggcccgaggccgccccgtgactgtgaccgagggagggagggctgcctgatccctctcctcgcctgccttctggaagacttcagaagattgagcctcactggtgccaggaagccaaagcttactttgtagaactgacactaaactacccgaaggacttaggtgctttgtgtacttaaccccaggacctccttactttttaatataaagagtgatgttgtatttcgtgttctgcactttttaatataaagagtgatgttgtatttc

SEQ ID NO: 97 - DNA, 597 bp, Homo sapiens
atggagaccggcaggcaaacaggcgtgtctgctgagatgctcgccatgccccgaggtctgaagggcagcaagaaggatggaatccctgaggacctagacgggaacttggaagcacccagggatcaggaaggtgagctcaggagtgaggatgtcatggacctcacagaaggtgacagtgaggcctcagcctcagctcctcctgcagccaaaagacggaaaacacatacgaaaggcaagaaggagagcaagcccaccgtggatgcggaggaagctcagaggatgacaaccctgctgtctgccatgtctgaggagcagctgtcccgctacgaagtgtgtcgccggtcagcgttcccgagagcacgcgttgcgggtctgatgcgggctatcactggcagttcggtgtcggagaacgcggccattgccatggctggaatagccaagctctttgttggagaggtggtggaagaggcactggacgtgtgtgagatgtggggagagacgcccccgctgcagcccaagcatttaagggaggccgttcgcaggttaaagcccaagggcctcttccccaacagcaactgcaaaagaatcatgttctag

SEQ ID NO: 98 - DNA, 1557 bp, Homo sapiens
tctagagtttttcactggagatttgtcagaatgagcctccagtccccatccagactcctggagctggcaggccagagcctgctgaggaaccagttcttgaccatcttcatcctggacgagctgcccagggaggtcttccctctgatgttcatggaggcctccagcatgagacattttgaggccctgaagctgatggtgcaggcctggcccttcctccgcctccctctgggatccctgatgaagacacctcatctggagaccttgcaagctgtgctgaagggacttgatacactgctggcccagaagcttcgccccaggaggtggaaacttcaagtgctggatttgcgggatgttgatgggaatttctggactatatggtctggagccagggccctctcctgctccccagaggccatgagtaagaggcagacagtggaggactatccaaggacgggagagcaccagcccttgaaggtgttcatagacctctgccaaaaggaaagtacactggatgaatgcctgagctacctctgcaggtggatccactacagaagaggtctagtgcacctgtgttgtaataaggtgcagaattactcaatgcccacttcaagtttcagaaatttattgaaaagggtatacccagacagtatccaggagttggaaattaagagaaagtgctctctgaataaaacaggaaagtttgccccttacttgagccagatgagcaatcttcgcaaactctttttagccttcggttatgacgatgagttatatgtaagcggccaacagcagttcgttcctgacttggactgtccattcctctgcctgtactaccctcagatgctttatataagaaagatcagtaatatcaaagagcacctggagcacctgctcaggtgcctcaagaaccccttgggaacctttatattctgtcatgcttacctagctgatcaggacatggagtgtctgtctcagtacccaagcctcagtcagctaaaggagctgcatctgattcatatcctaatgtggaccaccaatcttgagccccttggagctctgctagagaaagttgctgctactctcgagatcctcacgttaaaggactgtcagatccaggactcccagctcagggtcctcctgcctgccctgagccgctgctcccagctcaccaccttctactttcgcggaaatgagacctccacgaatgctctgaaagacctgctgtgtcacacaggtgggctgagcaagttaggtctggagttgtatcctgcccctctggagtgtcttgacaacaggggtcatgtcaattgggagatcctcgccccaattcgggctgagctgatgtgtacactcagggaagtcaggcagcccaagaggatcttttttggtcccatcccctgcccttcctgtggctcatggccatctgagaaagtggacttccatctttgctcttagtgaaggcctgattagtgggatggatatgctttcttcaggacccttaggcactaaaatctaggacacaggtgggtttttttgtttttttgttttttttttgat

SEQ ID NO: 99 - DNA, 4335 bp, Homo sapiens
gtctaatgtaaatccagcccagtgattacattagctgggcgctgattaggttaggatgttgcccaggtacaaggccaggatcttcgggacctggcttcatttggagttcagctaccaaaaggaaaccttcctctgggtcctggagtatttggcctgaaattgggaactcggaagttgctgctccagggcgctccctgcggagctccgccgcccgcctctccgcccggcctttcccggcgtccccacgcggggcgcaaccgcgagaaagaaacgcaggtcgcaccgtcagcgcccagagcagcgccagtttccgggcccgggctgctctcggagccatgagctgcggccgcccccctcccgacgtggacggcatgatcaccctcaaggtggacaacctgacctaccgcacctctcccgacagcttgaggcgcgtgttcgagaagtacgggcgcgtgggcgacgtgtacatcccgcgggagccccacaccaaggcgccccggggcttcgctttcgtccgctttcacgaccggcgcgacgcccaagacgccgaggccgccatggacggggcggagctggacggacgcgagctgcgggtgcaggtggcgcgctatggccgccgggacctgccccgcagccgccagggagagccacgcggcaggtccagaggcggcggctacggacggcggagccgcagctacgggcggcggagccgcagccccaggcggcgacaccgcagccgatcccggggtcccagctgctccaggtcccgcagccgatctcgctataggggttctcgctatagccggtctccctacagccgatctccttacagccggtcgcgctacagccgctctccctacagcagatctcgctacagggaatctcgctacggcggatctcactacagctcatctggttacagtaactctcgctacagccgatatcacagcagccggtctcactcgaagtctgggtcctccactagctctcgctctgcatcaacctccaaatcgagctctgcgcgacgatccaagtcctcctcggtctccaggtctcgctcgcggtccaggtcttcatctatgaccaggagtcctccccgggtatccaagaggaaatccaagtcaaggtcgcgatccaagaggccccccaagtctcctgaagaggaaggacagatgtcctcttaagaaaatgatgcatcaggaagcaacgtgatggaggacttgggggaaaaggatcacatactcagtctatggaagcaacgtccctggaagaagaggctgcctattgaaaaggttgtgtcacacttttctacctttttgccagtttgaagctttgcatcaggtggcaaaattcattctatgtgccgttttgttgttattcacattttattgtaacttaggaggtgaacgacctaagattacgttattgggtttggatatttgaggcaaaaatttatttttatttctatagtgatgactgttttggtttgaaatgaacagattggtaacctaatttgtggcctcctgacttttaaggaaacgtgtgcagccattacacacagcctaacgctgtcaagacattgcttcaacattgccttcattccttaaaaacctacaaaaggtggtgtaaattaatatggataattttatttacctccaggtctaaaaggtagtgtgacccaaatttgtataaagatttttcatgtgaaaggactggggttttagcaaacacaggtctaatctcttttgtgtttttgtgcaccaggcccggctgcgtagcagttgagtgatgctggttagctattaaggtggcctgttgcagtgcagagtgctgagctgcttcctgttttcttctgattgctcctggggaaaacacgccttgtcctgaagaacaaatggctgtccagtttattaaaatgcctgtcaactgcacttccagtcacccaggccttgcagataaataatggagcatgcagtgagcacatctagctgacgataatcacaccttttcccccgtcttttctgaaaaattgtaaatctgatcatatcaacatgtatgaacttaaaatatggagaatgttatggaagaaatagtttataagtttgttaagtacttataacatggtttatctttttgattattaattttttacgctaaccattgtttctgtagttaaaattgttttcttggtgttatcttttctcagaataaaattagaaacttttgatggaaagtaggttgttttattttctgtatgacttttggatatttgtacttttgagaaaattattagcaccaagtgtttctcaaaatataatttttaaaaaatccttaataggcttttagctatgtgctttattgttttatcacaatgcagtttatttgtagtttctctcttttttcctcacacctatggtttttttacttccaaaattattttcaaataatccatttttggctttcatcattatccctactagatgttatgtgttcttttgcaattgtttctgcttatacctttactagcaaagggaaaaataacaatttggtgtcaatgatctggtgacaataggattacattggagccaattgaataaatttattctttcaatcatatggatcttcattaatatttttgactgaattatcaattaaaaatatttcattccctttgtgcagaaactgctaaatccagggttcgattcttgaatgaactggcaaggtggctgtggtctgtagatatacatcccacattttgttgttataacagttagtagttagtattgctttcatatatagactccagaatctaaattttacgataatgacatttcttctggtcatgacaaatgtaatattttacaaatataaatctacgtagaatccaaagacacacacagagcagtcctgtctgagaaataaaaaatcaggacacccatggcatcgtagtagcccctcgcgtccagcaggtggcgaagggaggtgaggtatatttattaaatgggaccgagtgggacggggacggggcagccctaagggtagggaagcattgtcaatttctggggatagaatgagacccaggcatagctggagtttgaagctttgaagcaaaaatatctgtagaacatcttaaacgtgaccaaaatatgatgttaaaatcagcaactctttatacgttaaaaactttgaaacctgtggccgggcatggtggctcgcgcctgtaatcccagcactttgggaggccaaggcaggtggatcacctgaggtcaggagctcaagaccagcctgaccaacatggtgaaaccccgtctctactaaaaatacaaaaattagctgggcatggtggcgcatgcctataatcccagctactcggaggctgaggcaggataatcgcttgaaaccgggagacagaggttgtggtgagccgagatcacgccattgcactccagccggggcaacaagagcaaaacttcatctcaaaaaaaaaaaaaaaagaaaaaggaaaaaaacaaacaaacaacaactttgaaacctaggaacaattttggacctgttggagcagaaatgtgtagttgtcattgaaggtgtgaacataccctacctctactctacatgtggaagacctgacccacagaggacatgaacccatgaagaagagtgatgtgacatgttttgcattttattagtaccatgatctaaccaactgagctaactggccacaccattttatcctttataatgatcactatgagggctgagtgcagaatggagtgtatgtggtcaagagtggaagctgtgaggctattgcagtaatccagaaagaaaggtgacaatggcttagacttcagtggtagtgttggaaatggacataaatagacatattcaggaaatatattgatcttagatctaataagacacgtaatagattagaagtagggaaattaatgaaaaagcagaattaaggatgacttctcactggcttgaaatgctaaatggatgacggtgctgtttagtgagttggggaagactgggcgtggtgatgggtgtagaaattgagaaaggaaaatcaagaatttccagaactttcagagcaatgttaaatactttccttgatagtgggtcagtcatcatttataagtgcatttctaaaaatattgcttaattttgtttttgagctttgtataagtggaattttgtgtgtactgattattttgctcaatactatgtctttaagattctctgtattgttgcatgtggctgagtttatttttgatgctgtgtagtgttttactaattaataaatcataattcagtttttactgttctattgatgga

SEQ ID NO: 100 - DNA, 121 bp, Homo sapiens
gtctacggccataccaccctgaacgcgcccgatctcgtctgatctcggaagctaagcagggtcgggcctggttagtacttggacgggagaccgcctgggaataccgggtgctgtaggcttt

SEQ ID NO: 101 - DNA, 2008 bp, Homo sapiens
agtcacacaggggatgactgcaagtccttcgtaaccactccaggacgctgccagggcctgagaagggacaaactccaattgctccagttcaaactggtcctggcctggtcaaacggaggagacagagcagtgattcacagcaggacgtggaattgggacactcacccaggacacactatggtgcttcagccaagtgaaaggcactatcgagattggaaccacagaagatgtgccttgcttccccttcgccttctgccatgattgtaagtttcctgagacctccccagccatgcagaactctgacattatctctacggtagaattcaaccacacgggagaattactagcgacaggggacaaggggggtcgggttgtaatatttcaacgagagcaggagagtaaaaatcaggttcatcgtaggggtgaatacaatgtttacagcacattccagagccatgaacccgagttcgattacctgaagagtttagaaatagaagaaaaaatcaataaaataagatggctcccccagcagaatgcagcttactttcttctgtctactaatgataaaactgtgaagctgtggaaagtcagcgagcgtgataagaggccagaaggctacaatctgaaagatgaggagggccggctccgggatcctgccaccatcacaaccctgcgggtgcctgtcctgagacccatggacctgatggtggaggccaccccacgaagagtatttgccaacgcacacacatatcacatcaactccatatctgtcaacagcgactatgaaacctacatgtccgctgatgacctgaggattaacctatggaactttgaaataaccaatcaaagttttaatattgtggacattaagccagccaacatggaggagctcacggaggtgatcacagcagccgagttccacccccatcattgcaacaccttcgtgtacagcagcagcaaagggacaatccggctgtgtgacatgcgggcatctgccctgtgtgacaggcacaccaaattttttgaagagccggaagatccaagcaacagatcatttttctctgaaattatctcttcgatttcggatgtgaagttcagccacagtgggaggtatatcatgaccagggactacttgaccgtcaaagtctgggatctcaacatggaaaaccgccccatcgagacttaccaggttcatgactacctccgcagcaagctgtgttccctctatgaaaatgactgcatttttgataaatttgagtgtgtgtggaatgggtcagacagtgtcatcatgacaggctcctacaacaacttcttcaggatgttcgacagaaacaccaagcgtgatgtgacccttgaggcttcgagggaaaacagcaagccccgggctatcctcaaaccccgaaaagtgtgtgtggggggcaagcggagaaaagacgagatcagtgtcgacagtctggactttagcaaaaagatcttgcatacagcttggcatccttcagaaaatattatagcagtggcggctacaaataacctatatatattccaggacaaggttaactaggtggacaagttattacttaataatctcacatactgaatactagtcaaacaagtttttaaatgtttctttgggtcttcatttgatgcattgactttaatttccctatacaggaaatgattggaatagaattaaaaggagtccaacattcccagctccccagttctaagaaacttttgtcaaacccaataggtttgggacacttctgtttagaattgaaagctgccagctaacagtaattcttccatagttgacttgaacttctgatgcttttattgcccagttttctctggtgggtccagtgttttgttcctaggtgtctgctgcgataaaatgaggttgtctgtagtatttaaggagaaaagagataagttttttttaattaagcaattccatttgattgaaaaaaatcaacaaaaaataaacaccgtttactcttagacaaaaaaa

SEQ ID NO: 102 - DNA, 5653 bp, Homo sapiens
gacaaagagaactaatgctttgtgctgattcatatttgaatcgaggcattgggaaccctgtatgccttgtttgtggaaagaaccagtgacaccatcactgagcttcctaaaagttcgaagaagttagaggactatacactttcttttgaacttttataataaatatttgctctggtttttggaacccagggctgttagaggggtgagtgacaagtcttacaagtggccttattccaactccagaaattgcccaacggaactttgagattatatgcaatcgaaagtgacaggaaacatgccaactcaatccctcttaatgtacatggatgggccagaagtgattggcagctctcttggcagtccgatggagatggaggatgccttgtcaatgaaagggaccgctgttgttccattccgagctacacaagaaaaaaatgtcatccaaatcgaggggtatatgcccttggattgcatgttctgcagccagaccttcacacattcagaagaccttaataaacatgtcttaatgcaacaccggcctaccctctgtgaaccagcagttcttcgggttgaagcagagtatctcagtccgcttgataaaagtcaagtgcgaacagaacctcccaaggaaaagaattgcaaggaaaatgaatttagctgtgaggtatgtgggcagacatttagagtcgcttttgatgttgagatccacatgagaacacacaaagattctttcacttacgggtgtaacatgtgcggaagaagattcaaggagccttggtttcttaaaaatcacatgcggacacataatggcaaatcgggggccagaagcaaactgcagcaaggcttggagagtagtccagcaacgatcaacgaggtcgtccaggtgcacgcggccgagagcatctcctctccttacaaaatctgcatggtttgtggcttcctatttccaaataaagaaagtctaattgagcaccgcaaggtgcacaccaaaaaaactgctttcggtaccagcagcgcgcagacagactctccacaaggaggaatgccgtcctcgagggaggacttcctgcagttgttcaacttgagaccaaaatctcaccctgaaacggggaagaagcctgtcagatgcatccctcagctcgatccgttcaccaccttccaggcttggcagctggctaccaaaggaaaagttgccatttgccaagaagtgaaggaatcggggcaagaagggagcaccgacaacgacgattcgagttccgagaaggagcttggagaaacaaataagggcagttgtgcaggcctctcgcaagagaaagagaagtgcaaacactcccacggcgaagcgccctccgtggacgcggatcccaagttacccagtagcaaggagaagcccactcactgctccgagtgcggcaaagctttcagaacctaccaccagctggtcttgcactccagggtccacaagaaggaccggagggccggcgcggagtcgcccaccatgtctgtggacgggaggcagccggggacgtgttctcctgacctcgccgcccctctggatgaaaatggagccgtggatcgaggggaaggtggttctgaagacggatctgaggatgggcttcccgaaggaatccatctggataaaaatgatgatggaggaaaaataaaacatcttacatcttcaagagagtgtagttattgtggaaagtttttccgttcaaattattacctcaatattcatctcagaacgcatacaggtgaaaaaccatacaaatgtgaattttgtgaatatgctgcagcccagaagacatctctgaggtatcacttggagagacatcacaaggaaaaacaaaccgatgttgctgctgaagtcaagaacgatggtaaaaatcaggacactgaagatgcactattaaccgctgacagtgcgcaaaccaaaaatttgaaaagattttttgatggtgccaaagatgttacaggcagtccacctgcaaagcagcttaaggagatgccttctgtttttcagaatgttctgggcagcgctgtcctctcaccagcacacaaagatactcaggatttccataaaaatgcagctgatgacagtgctgataaagtgaataaaaaccctacccctgcttacctggacctgttaaaaaagagatcagcagttgaaactcaggcaaataacctcatctgtagaaccaaggcggatgttactcctcctccggatggcagtaccacccataaccttgaagttagccccaaagagaagcaaacggagaccgcagctgactgcagatacaggccaagtgtggattgtcacgaaaaacctttaaatttatccgtgggggctcttcacaattgcccggcaatttctttgagtaaaagtttgattccaagtatcacctgtccattttgtaccttcaagacattttatccagaagttttaatgatgcaccagagactggagcataaatacaatcctgacgttcataaaaactgtcgaaacaagtccttgcttagaagtcgacgtaccggatgcccgccagcgttgctgggaaaagatgtgcctcccctctctagtttctgtaaacccaagcccaagtctgctttcccggcgcagtccaaatccctgccatctgcgaaggggaagcagagccctcctgggccaggcaaggcccctctgacttcagggatagactctagcactttagccccaagtaacctgaagtcccacagaccacagcagaatgtgggggtccaaggggccgccaccaggcaacagcaatctgagatgtttcctaaaaccagtgtttcccctgcaccggataagacaaaaagacccgagacaaaattgaaacctcttccagtagctccttctcagcccaccctcggcagcagtaacatcaatggttccatcgactaccccgccaagaacgacagcccgtgggcacctccgggaagagactatttctgtaatcggagtgccagcaatactgcagcagaatttggtgagccccttccaaaaagactgaagtccagcgtggttgcccttgacgttgaccagcccggggccaattacagaagaggctatgaccttcccaagtaccatatggtcagaggcatcacatcactgttaccgcaggactgtgtgtatccgtcgcaggcgctgcctcccaaaccaaggttcctgagctccagcgaggtcgattctccaaatgtgctgactgttcagaagccctatggtggctccgggccactttacacttgtgtgcctgctggtagtccagcatccagctcgacgttagaaggaaaaaggcctgtgtcatatcaacacttatctaacagcatggcacaaaagagaaactatgagaattttattgggaatgcacattatcgaccaaatgacaaaaaaacttgattcactaattagggggaaaaaaggtcttggtggatgtcagtgcttactccccatgaaattaaattttacttcatcctttgagaagcgaatggtgaaagctactgaaataagctgtgattgtactgtacataaaacatatgaggaatctgcaaggaacactacagttgtgtaaagttgttctgttaacttttgtaccaaatagcaatacaaactagttggaacagttggaacttacatacatggggactggaaatctctattttgtccctgaataatatttttcttagaattgaccaaataagaagtggaatttttgcatacttgagcgctgctgaaaagaaatcatttgggttgggtggggcgggatgggggaaaagtatataatgcttgcacctcaggtaaaaatctgtaaatatctaagttgtaaacctgcttgttcaaatactgtgtgtattccttttctctaatgcagctcatcacttggagcagtttctgctattgtgctctttcatttaaaatgtatgtttttttttttttaaactgtcaatgattttgtattatgttgaatccacccaaatctattgttgtcttaaaattgttaatggaagtattgaccctctatgatatgtgctgcagatatcgaggtcagccattcggaagctggcagcattttatcgcaactttgagcatctcagatggggaaggcaccttcttcctcgcctctccagattgtcctggaacctccaggatccttgactgagggcgttgggatggcttgtaggatttttaagagagtgtgtctacagacaagcattttctctgtagagcagccacacgttgtataaacataaactgtatgtgcagttatttaaatttgtttctgtcaaattaatcatttttgttggacgattcagtggcgggggggttttgcctaaattagtatataaaaacaaaaatgctaaattatatctgtgaattgcaggtattggggaacagttttaagggaaattttggggggaacattttaggtttgtatttggtagtcttaatgtatctggcatttgggtgaactgtggacatactagagttgattatagacacattgattttgaataaggaactgctggccgagcccgctgggagtctagaaagagaaaatctgtttctagacctcagttattttcccatttttggttgttttgaagcagtaacatttttctcagtgcacatgcaatttgggttttagagaagatggccaccagctggcttcctagatattttaaacttttgttctttaatatgctgtccatggctgagtttattagtacatgggcttagtgaccacaaaatattttattaagaaactgtttcaaaaataaatttgcactgttcatttttctggcctcgctgttctccatagagcaagggtaatcctagaaaaaattttttttttttaaattatgcaacgtaagatgtcctccttgatagaagtcttagctcctgtgttacaagggagaactcatttgagatcagtctgttggcattgcaatgaagtgctttgtatcaggaaagtgtacactattgaccttttttcctgttcacaagctgagccatatgtacataatctagattttgttttcatagttttgcacttttatagcctatttttgaagattaacacatttgcaagatgattgactcaatctttgcctaatccaatgagtgttacagagagcttgctgtgactagaaccataaatcttaaagggggtatgtgataatagagggctggaatttaaacctgtatttaaaaaaaagaatcaccaaatctatttgaaaacaagtcgatttgtattatgctggaattttttgggctttcagatttctctttttaaccacatttctgaatgtataaaaataccaattattttcctacagccctttgtacttcaaaatatgtttttgtgtccatcagtattaactattggtatactactggttttatatttttttttctttgagacaacagtacatataatagaggtacaattcgttggatttttgtttatgtatttatttcattccagtttgatttattttaattgttgatacttaagttgtcgaacagtagacattacttgttttatttatgatatatttcagcttaaagttatgttattatatgtggaagtgtaaatatagatttggtgttttgcaaaaaaaaaaaaaaaaaaaa

SEQ ID NO: 103 - DNA, 2222 bp, Homo sapiens
ccaggtgcacagcgcatcgcccgaggctgtcaccgccctgccccgcccaccccagctgtcctggacccaggggcagggagaggctggacgccaggtgcgcggacacagaagcgtctaagcacagcttcctccttgccgctccgggaagtgggcagccagcccaggaaccagtaccacctgcaccatggggctgtcccggaaggagcaggtcttcttggccctgctgggggcctcgggggtctcaggcctcacggcactcattctcctcctggtggaggccaccagcgtgctcctgcccacagacatcaagtttgggatcgtgtttgatgcgggctcctcccacacgtccctcttcctgtatcagtggctggcgaacaaggagaatggcacgggtgtggtcagccaggccctggcctgccaggtggaagggcctggaatctcctcctacacttctaatgctgcacaggctggtgagagcctgcagggctgcttggaggaggcgctggtgctgatcccagaggcccagcatcggaaaacacccacgttcctgggggccacggctggcatgaggttgctcagccggaagaacagctctcaggccagggacatctttgcagcagtcacccaggtcctgggccggtctcccgtggacttttggggtgccgagctcctggccgggcaggccgaaggtgcctttggttggatcactgtcaactacggcttggggacgctggtcaagtactccttcactggagaatggatccagcctccggaggagatgctggtgggtgccctggacatgggaggggcctccacccagatcacgttcgtgcctgggggccccatcttggacaagagcacccaggccgattttcgcctctacggctccgactacagcgtctacactcacagctacctgtgctttggacgggaccagatgctgagcaggctcctcgtggggctggtacagagccgcccggctgccctgctccgtcacccgtgctacctcagcggctaccagaccacactggccctgggcccgctgtatgagtcaccctgtgtccacgccacgcccccgctgagcctcccccagaacctcacagttgaagggacaggcaaccctggagcctgcgtctcagccatccgggaacttttcaacttctccagctgccagggccaggaggactgcgcctttgacggggtctaccagcccccgctgcggggccagttctatgccttctccaacttctactacaccttccacttcctgaacctcacctccaggcagcccctgagcacggtcaacgccaccatctgggagttttgccagaggccctggaaactggtggaggccagctaccctgggcaggaccgctggctgcgggactactgtgcctcaggcctgtacatcctcaccctcctgcacgagggctacgggttcagcgaggagacctggcccagcctcgagttccgaaagcaggcgggcggtgtggacattggctggacactgggctacatgctgaacctgaccgggatgatcccggccgatgcgccggctcagtggcgggcagagagctacggcgtctgggtggccaaagtggtgttcatggtgctggccctggtggcggtggtgggggctgccttggtccagctcttctggttgcaggactagtgggaaggcggaggtgggcccccacagagcccacaggcagctgcgtcccggatgctggaggcttcctgagccctgagcgccgtggggccttgctctgtggctctgcccacggtcaggtgacagccacctccagggcaccgtcagggtggtgctggccacagaggctgcatgacctcccctcccggcgtccctcccccaacctccttccgcaactgggcttccagggccgtaggtgcctttctgcacacaggccgccaggactcgtggtgtctccaggctgtgtgactgcagggccacatgctgcctgcaaacagggcaagaccacggaggcacaggggtcctgctcctgatggggcctcaggaggggcggagaggggtggaagggagggagctgccccacctggacccccgctctccctgctgttgtctgagcagatggatggagtccaggcctgggggcttctgctgggccagcccggcctcccacacccacttggagggtgagactgcagtgggggttgtttttattaaaagcatcatggacacagca

SEQ ID NO: 104 - DNA, 891 bp, Homo sapiens
caggcccgggctgctctcggagccatgagctgcggccgcccccctcccgacgtggatggcatgatcaccctcaaggtggacaacctgacctaccgcacctctcccgacagcttgaggcgcgtgttcgagaagtacgggcgcgtgggcgacgtgtacatcccgctggagccccacaccaaggcgccccggggcttcgccttcgtccgctttcacgaccggagcgacgcgcaagacgccgaggccgccatggacggggcggtgctggacgaacgcgagctgcgtgtgcggatggcgcgctatggccgccgggacctgcctcgcagcggccaggaagagccacgcggcaggtcctggggcggccgctacggacggcggagcggcagccccagggggcgacaccgcagccaatcccggggtcccagctactctaggtcccgcagcagatctcactatggggggtctcgctatagcctgtctatctacaggagatctcgctacagccgatctccctacagacgatctcattacaggggatctcgctacggccgatctccctacagtcgatcttacagccggcatcactacagccgatctccctacagggaatctcgctacaggaggtctccctacatccggtcttcccgcagcaggtctccctaccgccgctctcactcgaagtctgggtctcactctgcatctccatcaacctccaaatccagctctccgtgaagatccaagtcctcctccatctccagatcttgctcatggtccaggtctagatctacgtccgggagtcctcccccgacatccaagagggaatccaagtccaggtcgcgatccaagagtcctcccaagtctcctgaaggggaaaaaggacaagtgccctcctag

SEQ ID NO: 105 - DNA, 1091 bp, Homo sapiens
gagccgatgcccgattccgcgcccgccatggccgacaaaatggacatgtctctggacgacatcattaaactgaaccggagccagcgaggcggccggggcgggggccggaaccgcgccgggccggctcccagggcggcccggcggtgggcgcaggccccgcgcgagtgaatcgaggcggcgggcccatccggaatccgcggccatcgccggcgcggccggcggaggcggcaggaaccgaccggcgccctacagcaggccaaaacaacttcccgacaagtggcagcacgatcttttcgacagtggcttcggcggtggtgccggcgtggagacaggtgggaaactgctggtgtccaatctggattttggagtctcagacgccgatattcaggaactctttgctgaatttggaacgctgaagaaggcggctgtgcactatgatcgctctggtcgcagcttaggaacagcagacgtgcactttgagcggaaggcagatgccctgaaggccatgaagcagtacaacggcgtccctctggatggccgccccatgaacattcagcttgtcacgtcacagattgacgcacagcggaggcctgcacagagcgtaaacagaggtggcatgactagaaaccgtggcgctggaggttttggtggtggtggaggcacccggagaggcacccgcggaggcgcccgtggaagaggcagaggtgccggcaggaattcaaagcagcagctttcggcagaggagctggatgcccagctggacgcctataatgcgagaatggacaccagttaaacagaccagcaaatccgcgtgcggaacaggacccaggcgtctcctcttgctccctggttggggggcggtggctggggctgtgcggccaatgatggatttgtttcttttatgttttaaaataggatttaaaaactcatgtaaaggttttttttttttctttttttttttttttaattctgaaacagacctgttttgtaccgagttatttttgggataaattttactggttgctgttgtggagaaggtggcgtttccaccttttccataataaaatagaaatgtgtgtagaactggaact

SEQ ID NO: 106 - DNA, 597 bp, Homo sapiens; identical to SEQ ID NO: 76

SEQ ID NO: 107 - DNA, 330 bp, Homo sapiens
agtctcagcgtggggtgaagcctagcagctatgaggatccattatcttctgtttgctttgctcttcctgtttttggtgcctgttccaggtcatggaggaatcataaacacattacagaaatattattgcagagtcagaggcggccggtgtgctgtgctcagctgccttccaaaggaggaacagatcggcaagtgctcgacgcgtggccgaaaatgctgccgaagaaagaaataaaaaccctgaaacatgacgagagtgttgtaaagtgtggaaatgccttcttaaagtttataaaagtaaaatcaaattacatttttttttcaaaaaaaa

SEQ ID NO: 108 - DNA, 1574 bp, Homo sapiens
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcgctggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggtctaggcccggtgagagactccacaccgcggagaactgccattctttcctgggcatcccggggatcccagagccggcccaggtaccagcagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatag

SEQ ID NO: 109 - DNA, 1710 bp, Homo sapiens
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcgctggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggccgtggcctctctttcgcggggaacacctggctggctacggaggggcgtgtctccgccccgccccctccaccgggctgaccggcctgggattcctgccttctaggtctaggcccggtgagagactccacaccgcggagaactgccattctttcctgggcatcccggggatcccagagccggcccaggtaccagcagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatag

SEQ ID NO: 110 - protein, 424 aa, Homo sapiens
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 111 - DNA, 768 bp, Homo sapiens
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggtctaggcccggtgagagactccacaccgcggagaactgccattctttcctgggcatcccggggatcccagagccggcccaggtaccagcagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatag

SEQ ID NO: 112 - protein, 160 aa, Homo sapiens
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQV

SEQ ID NO: 113 - DNA, 20 bp, Artificial sequence
  Synthetic
acccatcactggactggtgt

SEQ ID NO: 114 - DNA, 20 bp, Artificial sequence
  Synthetic
cacatcctcaaagagcctga

SEQ ID NO: 115 - DNA, 20 bp, Artificial sequence
  Synthetic
gctggaacaccttcagttgc

SEQ ID NO: 116 - DNA, 20 bp, Artificial sequence
  Synthetic
agttctccaaggggttctgg

SEQ ID NO: 117 - DNA, 21 bp, Artificial sequence
  Synthetic
gagacgtaggcttcggatctt

SEQ ID NO: 118 - DNA, 20 bp, Artificial sequence
  Synthetic
ggctgaattcaagtgggtct

SEQ ID NO: 119 - DNA, 20 bp, Artificial sequence
  Synthetic
tggaaatcaagtggcaaaaa

SEQ ID NO: 120 - DNA, 18 bp, Artificial sequence
  Synthetic
ctgcatgtggacgtggac

SEQ ID NO: 121 - DNA, 20 bp, Artificial sequence
  Synthetic
accaatggtgtttcacatgg

SEQ ID NO: 122 - DNA, 20 bp, Artificial sequence
  Synthetic
tgaataagggtgtggctgtg

SEQ ID NO: 123 - DNA, 20 bp, Artificial sequence
  Synthetic
cccacatcaaggaactggag

SEQ ID NO: 124 - DNA, 20 bp, Artificial sequence
  Synthetic
tgttggcatccaaggtcata

SEQ ID NO: 125 - DNA, 20 bp, Artificial sequence
  Synthetic
cgtggaacgtttttcctgtt

SEQ ID NO: 126 - DNA, 20 bp, Artificial sequence
  Synthetic
ggtgctgaaatcaacccact

SEQ ID NO: 127 - DNA, 22 bp, Artificial sequence
  Synthetic
ggtcccacctataggaatgttg

SEQ ID NO: 128 - DNA, 20 bp, Artificial sequence
  Synthetic
gaccaagcgtcttccttctg

SEQ ID NO: 129 - DNA, 20 bp, Artificial sequence
  Synthetic
gcgttcacctcttttccaag

SEQ ID NO: 130 - DNA, 20 bp, Artificial sequence
  Synthetic
gccatgtggatttctcgttt

SEQ ID NO: 131 - DNA, 22 bp, Artificial sequence
  Synthetic
tgaagcagatccattcttgaaa

SEQ ID NO: 132 - DNA, 20 bp, Artificial sequence
  Synthetic
accctgtaaatgcagcaagg

SEQ ID NO: 133 - DNA, 20 bp, Artificial sequence
  Synthetic
tgaatgtggaaaccaccaga

SEQ ID NO: 134 - DNA, 20 bp, Artificial sequence
  Synthetic
gttgagcctgtccctcagtc

SEQ ID NO: 135 - DNA, 20 bp, Artificial sequence
  Synthetic
accttcttcagtgggcacct

SEQ ID NO: 136 - DNA, 20 bp, Artificial sequence
  Synthetic
tgggaactgggagagacact

SEQ ID NO: 137 - DNA, 20 bp, Artificial sequence
  Synthetic
ccatagcagccaagatgatg

SEQ ID NO: 138 - DNA, 20 bp, Artificial sequence
  Synthetic
gaacttggtcaatccggaga

SEQ ID NO: 139 - DNA, 20 bp, Artificial sequence
  Synthetic
gcaggagagggataggaagc

SEQ ID NO: 140 - DNA, 20 bp, Artificial sequence
  Synthetic
cttgtgggggatcttgtcat

SEQ ID NO: 141 - DNA, 20 bp, Artificial sequence
  Synthetic
ctaaggcaaatggggtgaag

SEQ ID NO: 142 - DNA, 20 bp, Artificial sequence
  Synthetic
tctgggcccgagataaagta

SEQ ID NO: 143 - DNA, 20 bp, Artificial sequence
  Synthetic
gtccagctcatatcgggaaa

SEQ ID NO: 144 - DNA, 20 bp, Artificial sequence
  Synthetic
gctgcactccttttctggac

SEQ ID NO: 145 - DNA, 20 bp, Artificial sequence
  Synthetic
cttctgccgtgattgtgagg

SEQ ID NO: 146 - DNA, 20 bp, Artificial sequence
  Synthetic
ccagggggtctaggtaggag

SEQ ID NO: 147 - DNA, 20 bp, Artificial sequence
  Synthetic
aacctcctccttttccaagc

SEQ ID NO: 148 - DNA, 21 bp, Artificial sequence
  Synthetic
gcagtacctgtttagccacga

SEQ ID NO: 149 - DNA, 2375 bp, Homo sapiens
tcaggctcgctgtcgcgccattttgccggggtttgaatgtgaggcggagcggcggcaggagcgggtagtgccagctacggtccgcggctggggttccctcctccgtttctgtatccccacgagatcctatagcaatggaactcagcgatgcaaatctgcaaacactaacagaatatttaaagaaaacacttgatcctgatcctgccatccgacgtccagctgagaaatttcttgaatctgttgaaggaaatcagaattatccactgttgcttttgacattactggagaagtcccaggataatgttatcaaagtatgtgcttcagtaacattcaaaaactatattaaaaggaactggagaattgttgaagatgaaccaaacaaaatttgtgaagccgatcgagtggccattaaagccaacatagtgcacttgatgcttagcagcccagagcaaattcagaagcagttaagtgatgcaattagcattattggcagagaagattttccacagaaatggcctgacttgctgacagaaatggtgaatcgctttcagagtggagatttccatgttattaatggagtcctccgtacagcacattcattatttaaaagataccgtcatgaatttaagtcaaacgagttatggactgaaattaagcttgttctggatgcctttgctttgcctttgactaatctttttaaggtatggaatgcatcttggtgatatttttaaattaatattttaaattgcttgaatgtttgtatacatgttaagagaatgctttgaaagcttatttgatagataatgtttagagcatttcttttgaaaaatttcaaacctacacagaaatagagatgctatagtgaactcccatctattattacacaaatttaatggttagcaacgttttattttctctgttccctttcttaggaatgttataaaagcaaatcccaagcatcatatcatttcacccaagtattttaaagtatgtgtctctaaaggaagaaatacatatttttatttgactatgtttggagtttaattaattatatatttatacattattttacagtttacaggatactttcctgaatgtgttcttggtgtttgtgaaaatcctataagatttaaagaaatatatatatactttttttcttttttcttttttaaagacagggtctcactctcattgcccaggctggagtgcagtgagctattacagtgccactgcaatccagcctgggcaacagagcgaggtcccgtttcttaaaaaacatatatgtgtgtggcgtgtgtatatatatgtatatatattttttcattgtattattacggagactttcaaacatatatagaaagagcataatgaagcctgcatgtgcccagcttcaataattaccaatatcttgccagttttgtttcgtttctccttttattctctgtattgagcaagtcttagacatcatacgtttcacgcgtaagtaccttattctacatcattaaccagtaaggactttttaattaaccacaataccactatcacacctaataatagtaattccttatggatcttttctttagacctatttttgaaggcataaaagcagttgagtttctggagaatttttggatggtgattaatgacttgactggctgctcttcccagagctgtggcagctctcccgtagaagatggggtttgtattggcgcaccaagatctccaacagccagtgtgtgtttcccatctcttgtaggttccatcaatggtgagcaccagcctgaatgcagaagcgctccagtatctccaagggtaccttcaggcagccagtgtgacactgctttaaactgcatttttctaatgggctaaacccagatggtttcctaggaaatcacaggcttctgagcacagctgcattaaaacaaaggaagttctccttttgaacttgtcacgaattccatcttgtaaaggatattaaatgttgctttaacctgaaccttgagcaaattagttggtttgtgtgatcatacagttatgtgggtggcttctagtttgcaacttcaagggacaagtattaatagttcagtgtatggcgttggtttgtgttgagcgtttgcacggtttggataatcttaaattttgacggacactgtggagactttctgttactaaatccttttgttttgaagctgttgctatttgtatttctcttgtcctttatattttttgtctgtttatttacgcttttattggaaatgtgaataagtaaagaattacttgtgttacttgccaagcagtgcacatttcatagtttcaaatctgtaatcagcaataaaaatcctaaaatatgtacctaaaaaaaaaaaaaaaaaaaa

SEQ ID NO: 150 - DNA, 31 bp, Artificial sequence
  Synthetic
aggagtgatgataatttaatcagccgtgcaa

SEQ ID NO: 151 - DNA, 31 bp, Artificial sequence
  Synthetic
aggagtgatgatacttttatgagccgtgcaa

SEQ ID NO: 152 - DNA, 31 bp, Artificial sequence
  Synthetic
cctgtgggaggtaatccaatcatggaggcag

SEQ ID NO: 153 - DNA, 31 bp, Artificial sequence
  Synthetic
cctgtgggaggtactcctatgatggaggcag

SEQ ID NO: 154 - DNA, 31 bp, Artificial sequence
  Synthetic
ccaggtggagataattgaatcatgggggcag

SEQ ID NO: 155 - DNA, 31 bp, Artificial sequence
  Synthetic
ccaggtggagatacttgtatgatgggggcag

SEQ ID NO: 156 - DNA, 20 bp, Artificial sequence
  Synthetic
acccctcatggagaacctct

SEQ ID NO: 157 - DNA, 20 bp, Artificial sequence
  Synthetic
accctcttctcacagctcca

SEQ ID NO: 158 - DNA, 21 bp, Artificial sequence
  Synthetic
ttctcaggcttcaagatttgg

SEQ ID NO: 159 - DNA, 20 bp, Artificial sequence
  Synthetic
ctggagcttgcggaatttag

SEQ ID NO: 160 - DNA, 20 bp, Artificial sequence
  Synthetic
caccccaagttcgaggagat

SEQ ID NO: 161 - DNA, 20 bp, Artificial sequence
  Synthetic
agcgttggacacgtcaaata

SEQ ID NO: 162 - DNA, 28 bp, Artificial sequence
  Synthetic
cactcccctgcggcctgctgctggatga

SEQ ID NO: 163 - DNA, 30 bp, Artificial sequence
  Synthetic
ccaggagatgtaactctaatccaggtttgc

SEQ ID NO: 164 - DNA, 27 bp, Artificial sequence
  Synthetic
gagctcctggcgagcccggagtttctg

SEQ ID NO: 165 - DNA, 30 bp, Artificial sequence
  Synthetic
gtaactctaatccaggtttgcctagacagc

SEQ ID NO: 166 - DNA, 11 bp, Artificial sequence
  Synthetic
taaybbaatca

SEQ ID NO: 167 - DNA, 11 bp, Artificial sequence
  Synthetic
taayybaatca

SEQ ID NO: 168 - DNA, 11 bp, Artificial sequence
  Synthetic
taaybyaatca

SEQ ID NO: 169 - DNA, 11 bp, Artificial sequence
  Synthetic
taatttaatca

SEQ ID NO: 170 - DNA, 11 bp, Artificial sequence
  Synthetic
tacttttatga

SEQ ID NO: 171 - DNA, 11 bp, Artificial sequence
  Synthetic
taattgaatca

SEQ ID NO: 172 - DNA, 11 bp, Artificial sequence
  Synthetic
taatccaatca

SEQ ID NO: 173 - DNA, 10 bp, Artificial sequence
  Synthetic
tacttctatg

SEQ ID NO: 174 - DNA, 11 bp, Artificial sequence
  Synthetic
tactcctatga

SEQ ID NO: 175 - DNA, 31 bp, Artificial sequence; identical to SEQ ID NO: 150
  Synthetic

SEQ ID NO: 176 - DNA, 31 bp, Artificial sequence
  Synthetic
aatcacgtctttaaatcaatcactgacatgg

SEQ ID NO: 177 - DNA, 141 bp, Artificial sequence
  Synthetic
agtaattcaatcaacagacaagtgttatccaatcacgtctttaaatcaatcactgacatggagctggggctggatgaagattccatcagtaattcaatcaacagacaagtgttatccaatcacgtctttaaatcaatcact

SEQ ID NO: 178 - protein, 67 aa, Homo sapiens
MRIHYLLFALLFLFLVPVPGHGGIINTLQKYYCRVRGGRCAVLSCLPKEEQIGKCSTRGRKCCRRKK

SEQ ID NO: 179 - DNA, 19 bp, Artificial Sequence
  Synthetic
cuuacgcugaguacuucga

SEQ ID NO: 180 - DNA, 19 bp, Artificial Sequence
  Synthetic
gagccugcuuugagcggaa

SEQ ID NO: 181 - DNA, 19 bp, Artificial Sequence
  Synthetic
gcgcaaccucuccuagaaa

SEQ ID NO: 182 - DNA, 19 bp, Artificial Sequence
  Synthetic
caaaccuggauuagaguua

SEQ ID NO: 183 - DNA, 19 bp, Artificial Sequence
  Synthetic
gaugauuaguucagagaua

SEQ ID NO: 184 - DNA, 20 bp, Artificial Sequence
  Synthetic
ctagcctgttctggggaaga

SEQ ID NO: 185 - DNA, 20 bp, Artificial Sequence
  Synthetic
agtcggcacacttcttttgc

SEQ ID NO: 186 - DNA, 20 bp, Artificial Sequence
  Synthetic
gagcgcctcctacacaagag

SEQ ID NO: 187 - DNA, 20 bp, Artificial Sequence
  Synthetic
cggattctctgggagatttg

SEQ ID NO: 188 - DNA, 20 bp, Artificial Sequence
  Synthetic
tgtttgctttgctcttcctg

SEQ ID NO: 189 - DNA, 20 bp, Artificial Sequence
  Synthetic
cgcctctgactctgcaataa

SEQ ID NO: 190 - DNA, 20 bp, Artificial Sequence
  Synthetic
ctgtaaccttcccaggacca

SEQ ID NO: 191 - DNA, 20 bp, Artificial Sequence
  Synthetic
tcccttctggatctttttgg

SEQ ID NO: 192 - DNA, 19 bp, Artificial Sequence
  Synthetic
gtaccctgggatcgctgac

SEQ ID NO: 193 - DNA, 20 bp, Artificial Sequence
  Synthetic
ccgatccacaccgagtattt

SEQ ID NO: 194 - DNA, 20 bp, Artificial Sequence
  Synthetic
agattggggtggtgaatgtc

SEQ ID NO: 195 - DNA, 20 bp, Artificial Sequence
  Synthetic
tcctcaatccagtcctccag

SEQ ID NO: 196 - DNA, 20 bp, Artificial Sequence
  Synthetic
caagttcgagtttggggaga

SEQ ID NO: 197 - DNA, 20 bp, Artificial Sequence
  Synthetic
agccttcttgctgtgcttct

SEQ ID NO: 198 - DNA, 20 bp, Artificial Sequence
  Synthetic
gccaagtgtttccgatcatt

SEQ ID NO: 199 - DNA, 20 bp, Artificial Sequence
  Synthetic
cacgatggaagaaaggcatc

SEQ ID NO: 200 - DNA, 20 bp, Artificial Sequence
  Synthetic
gatcaatctccccatccaga

SEQ ID NO: 201 - DNA, 20 bp, Artificial Sequence
  Synthetic
tggcagagggtctctgtctt

(19) United States
(12) Patent Application Publication
(10) Pub. No.: US 2014/0242093 A1
(43) Pub. Date: Aug. 28, 2014

(54) METHODS AND COMPOSITIONS FOR MODULATING THE INNATE IMMUNE RESPONSE AND/OR MYOGENESIS IN A MAMMALIAN SUBJECT

(71) Applicants: Stephen J. Tapscott, Seattle, WA (US); Linda Geng, Plano, TX (US)

(72) Inventors: Stephen J. Tapscott, Seattle, WA (US); Linda Geng, Plano, TX (US)

(73) Assignee: FRED HUTCHINSON CANCER RESEARCH CENTER, Seattle, WA (US)

(21) Appl. No.: 14/236,003

(22) PCT Filed: Jul. 27, 2012
(86) PCT No.: PCT/US2012/048557

§ 371 Date: May 2, 2014

Related U.S. Application Data
(60) Provisional application No. 61/513,456, filed on Jul. 29, 2011.
(60) Provisional application No. 61/513,467, filed on Jul. 29, 2011.
(60) Provisional application No. 61/556,099, filed on Nov. 4, 2011.

(51) Int. Cl. C07K 16/18; G01N 33/569; C12Q 1/68; C12N 15/113

(52) U.S. Cl. CPC C07K 16/18; C12N 15/113; G01N 33/56966; C12Q 1/6881
USPC 424/172.1; 514/44 A; 536/24.31; 530/389.1; 435/375; 435/7.1; 435/6.11; 536/24.33

(57) ABSTRACT

In one aspect, the present invention relates to methods for increasing, decreasing or maintaining the innate immune response in a mammalian subject comprising modulating the expression of DUX4-fl, or modulating the expression of beta-defensin 3 (DEFB103). In another aspect, the present invention relates to methods for increasing, decreasing or maintaining myogenesis or muscle differentiation in a mammalian subject comprising modulating the expression of beta-defensin 3 (DEFB103). In additional aspects, the present invention involves diagnostic methods based on assessment of identified biomarkers.

This application claims priority to U.S. Application Nos. 61/513,456 and 61/453, 467 filed on Jul. 29, 2011, U.S. Application No. 61/556,099 filed on Nov. 4, 2011, the entire contents of which are hereby incorporated by reference without disclaimer.

This Invention was made with government support under NS069539, AR045113 and AR0045203 awarded by National Institutes of Health. The government has certain rights in the invention.

### FIELD OF THE INVENTION

The present invention relates generally to medicine, diagnostic and therapeutic methods. In particular, embodiments are directed to the diagnosis and treatment of DUX-4 related disorders, such as muscular dystrophy, autoimmune diseases, infection, and cancer.

### BACKGROUND

Immunity can generally be classified as innate immunity or as adaptive immunity Innate immune responses typically occur immediately upon infection to provide an early barrier to infectious disease whereas adaptive immune responses occur later with the generation of antigen-specific effector cells and often long term protective immunity.

The innate immune system, also known as non-specific immune system and first line of defense, comprises the cells and mechanisms that defend the host from infection by other organisms in a non-specific manner. This means that the cells of the innate system recognise and respond to pathogens in a generic way, but unlike the adaptive immune system, it does not confer long-lasting or protective immunity to the host. Innate immune systems provide immediate defense against infection, and are found in all classes of plant and animal life. The innate immune system is thought to constitute an evolutionarily older defense strategy.

There remains a need to identify strategies to modulate immune activation, including control of unwanted activation of the innate immune response or increase desired innate immune response.

Facioscapulohumeral dystrophy (FSHD) is the third most common muscular dystrophy. The mutation that causes FSHD was identified nearly 20 years ago (Wijmenga et al., 1992), yet the molecular mechanism(s) of the disease remains elusive. The most prevalent form of FSHD (FSHD1) is caused by the deletion of a subset of D4Z4 macrosatellite repeats in the subtelomeric region of chromosome 4q. Unaffected individuals have 11-100 of the 3.3 kb D4Z4 repeat units, whereas FSHD1 individuals have 10 or fewer repeats. At least one repeat unit appears necessary for FSHD because no case has been identified with a complete deletion of D4Z4 repeats (Tuplet et al., 1996). Each repeat unit contains a copy of the double homeobox retrogene DUX4 (Clapp et al., 2007; Gabriels et al., 1999; Lyle et al., 1995), and inappropriate expression of DUX4 was initially proposed as a possible cause of FSHD. This was supported by the observations that repeat contraction is associated with decreased repressive epigenetic marks in the remaining D4Z4 units (van Overveld et al., 2003; Zeng et al., 2009), and that overexpression of the DUX4 protein in a variety of cells, including skeletal muscle, causes apoptotic cell death (Kowaljow et al., 2007; Wallace et al., 2011; Wuebbles et al., 2010). However, initial attempts to identify DUX4 mRNA transcripts in FSHD muscle were unsuccessful, leading to the suggestion that other genes in the region were causative for FSHD (Gabellini et al., 2002; Klooster et al., 2009; Laoudj-Chemvesse et al., 2005; Reed et al., 2007).

Currently, the diagnostic test for FSHD1 requires pulse-field gel electrophoresis and Southern blotting to detect the contraction of the D4Z4 repeats, and there are no commercially available diagnostic tests for FSHD2.

### SUMMARY OF THE INVENTION

In accordance with the foregoing, in one aspect, the invention provides a method of inhibiting the innate immune response in a mammalian subject in need thereof, comprising administering to the mammalian subject an agent capable of inducing, or increasing the level of DUX4-fl expression in a population of cells in the mammalian subject.

In another aspect, the invention provides a method of inhibiting the innate immune response in a mammalian subject in need thereof, comprising administering to the mammalian subject an agent capable of inducing, or increasing the level of DEFB103A and/or DEFB103B expression in a population of cells in the mammalian subject.

In another aspect, the invention provides a method of increasing or maintaining the innate immune response in a mammalian subject in need thereof, comprising administering to the mammalian subject an agent capable of inhibiting, or suppressing the level of DUX4-fl expression, or an agent capable of inhibiting DUX4-fl mediated transcription activation in a population of cells in the mammalian subject.

In another aspect, the invention provides a method of inducing one or more testis expressed genes in a non-testis cell type comprising contacting the non-testis cell type with an agent capable of inducing, or increasing the level of DUX4-fl expression in a population of cells.

In accordance with the foregoing, in one aspect, the invention provides a method of determining the presence of, or risk of developing, Facioscapulohumeral dystrophy (FSHD) in a mammalian subject. The method in accordance with this aspect comprises: (a) determining the presence or amount of at least one FSHD biomarker in a biological test sample obtained from a mammalian subject, wherein the at least one FSHD biomarker comprises a gene product of a DUX-4-fl induced gene; and (b) comparing the presence or amount of the biomarker determined in step (a) with a reference standard or control sample, wherein an increase in the presence or amount of the FSHD biomarker determined in the test sample in comparison to the reference standard or control sample is indicative of the presence of FSHD, or increased risk of developing FSHD, in the mammalian subject.

In another aspect, the invention provides an isolated polynucleotide probe for detecting an FSHD biomarker, or a polynucleotide primer for amplifying at least a portion of an FSHD biomarker, wherein the nucleic acid probe or primer has a length of from at least 10 nucleotides to 200 nucleotides (i.e., from 10-50, 50-100, nucleotides, or 15, 20, 50, 75, 100, 150, or 175 nucleotides in length) and specifically hybridizes to the nucleic acid sequence of at least one FSHD biomarker set forth in TABLE 1 or TABLE 2.

In another aspect, the invention provides an isolated population of polynucleotide probes comprising a plurality of polynucleotides each complementary and hybridizable to a sequence of at least two different FSHD biomarkers selected from any one of TABLE 1 or TABLE 2.

In another aspect, the invention provides an isolated antibody that specifically binds to an FSHD polypeptide biomarker encoded by a nucleic acid set forth in TABLE 1 or TABLE 2. In another aspect, the invention provides a kit comprising one or more detection reagents for detecting one or more FSHD biomarkers set forth in TABLE 1 or TABLE 2 for use in an assay to determine the presence or risk of FSHD in a biological sample obtained from a mammalian subject.

In another aspect, the invention provides a nucleic acid molecule comprising an expression cassette comprising a promoter operationally linked to a reporter gene or selectable marker, wherein the promoter contains at least one DUX4-responsive element comprising the consensus sequence “TAAYBBAATCA” (SEQ ID NO:166).

In another aspect, the invention provides a method of detecting the presence of DUX4-fl protein in a cell sample comprising introducing a nucleic acid molecule comprising an expression cassette comprising a promoter operationally linked to a reporter gene or selectable marker, wherein the promoter contains at least one DUX4-responsive element comprising the consensus sequence “TAAYBBAATCA” (SEQ ID NO:166), and assaying the cell for expression of the reporter gene, or selecting for growth under conditions requiring expression of the selectable marker.

In another aspect, the invention provides a method of identifying an inhibitor of DUX4-fl induced expression. The method in accordance with this aspect of the invention comprises: (a) contacting a cell containing (i) an expression cassette comprising a promoter operationally linked to a reporter gene or selectable marker, wherein the promoter contains at least one DUX4-responsive element comprising the consensus sequence “TAAYBBAATCA” (SEQ ID NO:166), and (ii) DUX4-fl polypeptide, with a candidate inhibitory agent; and (b) determining whether the cell expresses the reporter gene or selectable marker in the presence and absence of the candidate inhibitory agent, wherein the absence of expression of the reporter gene or selectable marker in the presence of the inhibitory agent indicates that the agent is an inhibitor of DUX4-fl induced expression.

In further embodiments, methods may also involve determining the presence or absence of a polymorphism resulting in a functional polyadenylation sequence operationally linked to exon 3 of the DUX4 gene. The determination may involve genotyping a biolodical sample. A determination of the absence of a functional polyadenylation sequence operationally linked to exon 3 may indicate the subject does not have a genetic predisposition to develop or is not suffering from FSHD, while the presence of the sequence may indicate a predisposition toward developing the disease (or the presence of the disease already). In certain embodiments, the polymorphism is described in PCT/US2011/048318, which has been published as WO/2012/024535, which is hereby incorporated by reference.

Embodiments discussed in the context of methods and/or compositions of the invention may be employed with respect to any other method or composition described herein. Thus, an embodiment pertaining to one method or composition may be applied to other methods and compositions of the invention as well.

As used herein the terms “encode” or “encoding” with reference to a nucleic acid are used to make the invention readily understandable by the skilled artisan; however, these terms may be used interchangeably with “comprise” or “comprising” respectively.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows the results of RT-PCR validation of DUX-fl induced target genes shown to be upregulated in the expression microarray, (−) unstransfected cells; (+) transfected cells, as described in Example 1;

FIG. 2A illustrates the structure of the luciferase reporter construct containing a 31 bp DUX binding site (obtained from genomic regions of TRIM48 or ZCAN4 genes) located upstream of an SV40 promoter cloned into pGL3-promoter reporter vector, as described in Example 2;

FIG. 2B graphically illustrates the results of human rhabdomyoscaroma cell line RD transfected with the reporter construct containing the DUX4 binding site from TRIM48. Cells were co-transfected with the reporter construct and DUX4-fl or DUX4-s. pCS2-β galactosidase (beta gal) was used to balance DNA amount in control condition. TRIM48mut=construct containing a mutated binding site. Luciferase activity was set relative to control, as described in Example 2;

FIG. 2C graphically illustrates the results of human rhabdomyoscaroma cell line RD transfected with the reporter construct containing the DUX4 binding site from ZCAN4. Cells were co-transfected with the reporter construct and DUX4-fl or DUX4-s. pCS2-β galactosidase (beta gal) was used to balance DNA amount in control condition. ZSCAN4mut=construct containing a mutated binding site. Luciferase activity was set relative to control, as described in Example 2;

FIG. 2D shows the relative luciferase activity in the presence of DUX4-fl from a reporter construct in which the 31 bp DUX4 binding site was inserted in reverse orientation upstream of the SV40 promoter, as described in Example 2;

FIG. 2E shows the relative luciferase activity from a reporter construct in which the 31 bp DUX4 binding site was inserted in the original orientation, but moved downstream of the reporter gene, as described in Example 2;

FIG. 3 is a Heat map showing expression of cancer testis antigens (CTA) in HCT116 cells under conditions that activate DUX4-fl expression (i.e. treatment with the demethylating agent 5-azacytidine). The relative expression of the CTA in each row was measured by RT-PCR and represented as high (yellow, or light shading) or low (black, dark shading). The first column shows very low expression of CTAs in HCT116 that are not treated (−) and the second column shows a robust induction after treatment with azacytidine (+), a condition that induces expression of DUX4-fl, as described in Example 4;

FIG. 4 graphically illustrates that Pargyline decreases the amount of DUX4 mRNA in FSHD muscle cells. FSHD muscle cells that express endogenous DUX4-fl mRNA were treated with the MAO inhibitor Pargyline that has been reported to inhibit the histone demethylase LSD1, or with another MAO inhibitor tranylcypromine that has a different spectrum of activity on demethylase. The pargyline decreases the abundance of DUX4-fl mRNA in a dose dependent manner as measured by quantitative RT-PCR, as described in Example 6;

FIG. 5 graphically illustrates that Pargyline has a dose dependent inhibition of DUX4 mRNA expression in FSHD muscle cells. Cultured FSHD muscle cells were differentiated for 48 hours in differentiation medium (DM) with varying amounts of pargyline and the amount of DUX4-fl mRNA was measured by RT-PCR. There was a dose dependent inhibition of DUX4 expression (top panel). Middle panel is a no RT control and bottom panel is a GAPDH loading control, as described in Example 6;

FIG. 6 demonstrates that the protein synthesis inhibitor cycloheximide (chx) prevents decay of the DUX4 mRNA, as described in Example 6;

FIG. 7A graphically illustrates the expression levels (as determined by real-time PCR analysis of cultured myoblasts) of innate immune responder IFIH1 after infection with lenti-GFP or lenti-DUX4-fl, as described in Example 8;

FIG. 7B graphically illustrates the expression levels (as determined by real-time PCR analysis of cultured myoblasts) of secreted factor DEFB103 after infection with lenti-GFP or lenti-DUX4-fl, as described in Example 8;

FIG. 8A-8B graphically illustrates the expression levels (as determined by real-time PCR analysis of cultured myoblasts) of innate immune responders IFIH1 (FIG. 8A) and ISG20 (FIG. 8B) after infection with lenti-GFP in either media supplemented with human β-defensin 3 peptide or conditioned media (CM) from lenti-DUX4-fl, as described in Example 8;

FIG. 9 graphically illustrates the endogenous expression of DEFB103 in control testis and skeletal muscle tissues, FSHD muscle biopsies, and cultured FSHD and control muscle cells, as described in Example 8;

FIG. 10 graphically illustrates the upregulation of myostatin (MSTN) in β-defensin 3 peptide (DEFB103) treated myoblasts cultured in growth media, as described in Example 8;

FIG. 11A-11G graphically illustrates the expression levels of various muscle marker genes (FIG. 11A: ACTA1; FIG. 11B: CKM; FIG. 11C: CASQ2; FIG. 11D: MYH2; FIG. 11E: TNNT3; FIG. 11F: MYG6; and FIG. 11G: DESMIN) in response to human β-defensin when added to myoblasts cultured in differentiation media. MYG6 (FIG. 11F) and DESMIN (FIG. 11G) were included as genes that were unchanged on the arrays, as described in Example 8.

FIG. 12A-12B. UPF1 knock-down increases the abundance of the DUX4 mRNA and the expression of the DUX4 target gene ZSCAN4. A. Semiquantitative RT-PCR showing DUX4 expression in growth medium (GM) and differentiation medium (DM) from FSHD muscle cells. siUPF1 represents cells transfected with an si RNA to knock-down UPF1; siluc is the control si RNA to luciferase which is not expressed in the cells. DUX4 mRNA is detected in DM and is at higher levels in cells with the UPF1 knockdown. B. Determination by quantitative RT-qPCR of the levels of the DUX4 target gene ZSCAN4, as described in Example 9.

### DETAILED DESCRIPTION

Unless specifically defined herein, all terms used herein have the same meaning as they would to one skilled in the art of the present invention.

As used herein the term “the innate immune response” refers to the cellular pathways that respond to pathogen associated molecular patterns and activate a defense response through the RIG-1-like receptors, the toll-like receptors, or other pathogen associated molecular pattern receptors to activate interferon, NF-kapa-B, STAT, IRF and other response pathways that protect against pathogen infection. Indicators of the activation of the innate immune response include increased expression and/or phosphorylation of IRF family members, increased expression of the RIG-I like receptors, and increased expression of interferons and/or chemokines.

The terms “percent identity” or “percent identical,” as applied to polypeptide sequences, such as the polypeptides encoded by the DUX4-fl induced genes set forth in TABLES 1 and 2, or a portion thereof, is defined as the percentage of amino acid residues in a candidate protein sequence that are identical with the subject protein sequence (such as the amino acid sequence encoded by SEQ ID NO:1, or a portion thereof comprising at least 10 consecutive amino acid residues) after aligning the candidate and subject sequences to achieve the maximum percent identity. For example, percentage identity between two protein sequences can be determined by pairwise comparison of the two sequences using the bl2seq interface at the Web site of the National Center for Biotechnology Information (NCBI), U.S. National Library of Medicine, 8600 Rockville Pike, Bethesda, Md. 20894, U.S.A. The bl2seq interface permits sequence alignment using the BLAST tool described by Tatiana et al. (1999). The following alignment parameters are used: Matrix=BLOSUM62; Gap open penalty=11; Gap extension penalty=1; Gap x_dropff=50; Expect=10.0; Word size=3; and Filter=off. In some embodiments, the FSHD polypeptide biomarkers comprise at least 90%, or at least 95%, or at least 99% identity to the polypeptides encoded by the DUX4-fl induced genes set forth in TABLES 1 or 2, including naturally occurring variants thereof. The terms “percent identity” or “percent identical,” as applied to nucleic acid molecules, is the percentage of nucleotides in a candidate nucleic acid sequence that are identical with a subject nucleic acid molecule sequence (such as the nucleic acid molecule sequence set forth in SEQ ID NO:1, or a portion thereof comprising at least 20 consecutive nucleotides) after aligning the sequences to achieve the maximum percent identity, and not considering any nucleic acid residue substitutions as part of the sequence identity. No gaps are introduced into the candidate nucleic acid sequence in order to achieve the best alignment. Nucleic acid sequence identity can be determined in the following manner. The subject polynucleotide molecule sequence is used to search a nucleic acid sequence database, such as the Genbank database, using the program BLASTN version 2.1 (based on Altschul et al., 1997). The program is used in the ungapped mode. Default filtering is used to remove sequence homologies due to regions of low complexity as defined in Wootton and Federhen (1996). The default parameters of BLASTN are utilized. In some embodiments, the FSHD gene biomarkers comprise at least 90%, or at least 95%, or at least 99% identity to the nucleic acid sequences of the DUX4-fl induced genes set forth in TABLES 1 or 2, including naturally occurring variants thereof.

As used herein, the term “healthy human subject” refers to an individual who is known not to suffer from FSHD, such knowledge being derived from clinical data on the individual. As used herein, the term “DUX4-fl induced gene product” refers to a gene product (mRNA or polypeptide) expressed from a gene that is induced at least 2-fold (i.e. at least 3-fold, at least 5-fold, at least 8-fold, at least 10-fold, at least 16 fold or greater) in the presence of DUX4-fl, including genes driven by a promoter that is directly bound by DUX4-fl as well as genes that are induced indirectly by DUX4-fl. In some embodiments, the DUX4-fl induced genes contain one or more DUX4-fl responsive element(s) which are directly bound by DUX4-fl. As used herein, the term “DUX4-fl” encompasses naturally occurring DUX4-fl protein that is isolated from a human subject (i.e. SEQ ID NO:110, or a naturally occurring variant thereof, encoded by at least one of SEQ ID NO:108 or SEQ ID NO:109, or a naturally occurring variant thereof), as well as cultured cells making DUX4-fl, or made by recombinant DNA technology (e.g., in eukaryotic expression systems (e.g., COS cells)), in yeast, mammalian, or in bacterial expression systems). The term “variant of DUX4-fl” refers to a polypeptide comprising at least 90%, or at least 95%, or at least 99% identity to DUX4-fl polypeptide, set forth as SEQ ID NO:110, including naturally occurring variants thereof.

As used herein, the term “DUX-s” encompasses naturally occurring DUX4-s protein that is isolated from a human subject (i.e. SEQ ID NO:112, or a naturally occurring variant thereof, encoded by DUX4-s cDNA (Genbank No. HQ266762) (SEQ ID NO:111). The term “variant of DUX4-s” refers to a polypeptide comprising at least 90%, or at least 95%, or at least 99% identity to DUX4-s polypeptide, set forth as SEQ ID NO:112, including naturally occurring variants thereof.

As used herein, the term “FSHD gene marker” refers to an entire gene, or portion thereof, such as an EST derived from that gene, the expression or level of which (including mRNA or protein) is induced in the presence of DUX4-fl.

As used herein, the term “FSHD gene marker-derived polynucleotides” refers to the RNA transcribed from a marker gene, any cDNA or cRNA produced therefrom, and any nucleic acid derived therefrom, such as synthetic nucleic acid having a sequence derived from the gene corresponding to the marker gene.

As used herein, the term “biological sample” refers to any type of material of biological origin isolated from a subject, including, for example, DNA, RNA, protein, such as, for example, blood, plasma, serum, fecal matter, urine, semen, bone marrow, bile, spinal fluid, tears, saliva, muscle biopsy, organ tissue or other material of biological origin known by those of ordinary skill in the art.

As used herein, the term “antibody” encompasses antibodies and antibody fragments thereof, derived from any antibody-producing mammal (e.g., mouse, rat, rabbit, and primate including human), that specifically bind to FSHD biomarker polypeptides or portions thereof. Exemplary antibodies include polyclonal, monoclonal and recombinant antibodies; multispecific antibodies (e.g., bispecific antibodies); humanized antibodies; murine antibodies; chimeric, mouse-human, mouse-primate, primate-human monoclonal antibodies; and anti-idiotype antibodies, and may be any intact molecule or fragment thereof. As used herein, “a mammalian subject” includes all mammals, including without limitation humans, non-human primates, dogs, cats, horses, sheep, goats, cows, rabbits, pigs and rodents.

As used herein, the term “operatively linked” refers to a juxtaposition wherein the components so described are in a relationship permitting them to function in their intended manner. For example, a promoter sequence is operatively linked to a coding sequence if the promoter sequence promotes transcription of the coding sequence.

As used herein, the term “vector” is a nucleic acid molecule, preferably self-replicating, which transfers and/or replicates an inserted nucleic acid molecule into and/or between host cells.

As used herein, the term “nucleic acid sequences allowing for autonomous replication” refers to a polynucleotide comprising an origin of replication (generally referred to as an on sequence) which allows for replication of the polynucleotide in the appropriate host cell.

As used herein, the term “nucleic acid sequences allowing for selection” refers to polynucleotides encoding any protein that provides a phenotypic marker, for example, a protein that is necessary for cell growth, or resistance to a toxin, or a protein providing a surface antigen for which specific antibodies/ligands are available.

As used herein, the term “therapeutically effective amount” is an amount of an agent of the invention that alleviates, totally or partially, the pathophysiological effects of at least one of FSHD, myotonic dystrophy or Huntington's disease; or of an autoimmune disease such as Systemic Lupus Erythermatosis, Aicardi-Goutieres Syndrome or Multiple Sclerosis. The amount will depend on, for example, the subject size, gender, magnitude of the associated condition or injury, and the like. For a given subject in need thereof a therapeutically effective amount can be determined by those of ordinary skill in the art by methods known to those of ordinary skill in the art.

As used herein, the term “treat” and all its forms and tenses refer to both therapeutic treatment and prophylactic or preventative treatment.

### I. FACIOSCAPULOHUMERAL DYSTROPHY (FSHD)

It has been known for about 20 years that FSHD is caused by the shortening of a macrosatellite repeat array on chromosome 4, but the molecular mechanism leading to muscle pathology has been elusive and controversial. It was recently determined that the DUX4 retrogene contained in these repeats is the likely cause of FSHD, based on genetic studies that identified polymorphisms that create a DUX4 polyadenylation site as necessary for a D4Z4 contraction to cause FSHD (Lemmers et al., 2010). It has also been shown that a subset of individuals with clinical features of FSHD do not have contracted D4Z4 repeats on chromosome 4 but do have decreased repressive heterochromatin at the D4Z4 repeats (de Greef et al., 2009) (FSHD2), indicating that loss of repressive chromatin at D4Z4 is the primary cause of FSHD. High sensitivity RT-PCR assays detect DUX4 mRNA specifically in FSHD muscle (Dixit et al., 2007; Snider et al., 2010). It has also been shown that DUX4 is normally expressed in germ cells and epigenetically repressed in healthy somatic tissues, but the occasional escape from epigenetic repression of FSHD muscle cells results in bursts of DUX4 in a small fraction of nuclei (Snider et al., 2010). Still, a major problem with the hypothesis that DUX4 expression causes FSHD has been the extremely low abundance of the mRNA and inability to reliably detect the protein in FSHD biopsy samples.

Our prior work demonstrated that the low abundance of DUX4 in FSHD muscle cells represents a relatively high expression in a small subset of nuclei (Snider et al., 2010, supra). However, it remained unclear whether the low expression of DUX4 in FSHD muscle has a biological consequence that might drive the pathophysiology of FSHD.

### II. DUX4

DUX4 belongs to the double-homeobox transcription factor family, and the biological role of this large class of DNA-binding proteins is largely unknown. The coding sequence of the DUX4 retrogene has been conserved in primates (Clapp et al., 2007), but whether this retrogene has a normal physiological function is unknown. Previously the inventors found that DUX4 is normally expressed at high levels in germ cells of human testes and is epigenetically repressed in somatic tissues (Snider et al., 2010), whereas the epigenetic repression of the DUX4 locus in somatic tissues is less efficient in both FSHD1 and FSHD2, resulting in DUX4 expression in FSHD muscle cell nuclei. The germline-specific expression pattern of DUX4 is similar to that of other double homeodomain proteins (Booth and Holland, 2007; Wu et al., 2010). The function of this distinct family of DNA-binding proteins is unknown, but their shared tissue expression pattern may indicate a possible role for double homeodomain transcription factors in reproductive biology.

As described herein in Examples 1-3, the present inventors have now discovered that DUX4 regulates the expression of genes involved in germline and early stem cell development. As described herein, the genes regulated by DUX4 are reliably detected in FSHD muscle but not in controls, providing direct support for the model that misexpression of DUX4 is a causal factor for FSHD. As described in Example 1, through the use of expression arrays and chromatin immunoprecipitation combined with high throughput sequencing the inventors have identified DUX4 target genes that are bound and regulated by DUX4. As further described herein, DUX4 regulates germline and stem cell genes, which is consistent with its normal expression pattern and indicates a physiological role for DUX4 in germ cell and reproductive biology. As described in Example 2, the inventors identified the consensus binding site for DUX4, a double homeodomain motif, and further demonstrate that DUX4 binds to and activates transcription from endogenous retrotransposon LTRs of the MaLR family. As described in Example 3, the inventors have determined that the transcriptional targets of DUX4 are aberrantly expressed in biopsies of FSHD skeletal muscle but not in control muscle biopsies. Therefore, the low level of DUX4 expression in FSHD is sufficient to effect numerous downstream changes and activate genes of germ cell and early development in postmitotic skeletal muscle. These findings provide direct support for DUX4 as the causal factor for FSHD, and also provide valuable biomarkers to assess the presence or risk of FSHD, a disease that has been difficult to diagnose with genetic testing.

As described in Example 4, the inventors have determined that DUX4-fl activates expression of multiple cancer testis antigens and gene families in FSHD muscle and DUX4-fl expression correlates with expression of cancer testis antigens (CTAs) in a cancer cell and CTA family members are induced by DUX4-fl in dendritic cells.

As described in Example 5, the inventors have discovered that agents that increase chromatin mediated repression, such as agents that inhibit LSD1 activity, are useful to suppress DUX4 and are candidate therapeutic agents for FSHD. Such agents are believed to also have application to other diseases, such as myotonic dystrophy or Huntington's disease, where increasing chromatin mediated suppression of the mutant allele would have to herapeutic benefit. As further described in Example 5, the inventors have also discovered that an agent that modifies translation dependent nonsense mediated decay stabilizes DUX4 mRNA levels. Therefore, approaches that block translation dependent nonsense mediated decay can be used to increase DUX4 mRNA and agents that enhance nonsense mediated decay can be used to enhance the degradation of DUX4 mRNA, which provides a candidate therapy for FSHD.

As described in Example 6, DUX4 binds and activates LTR elements from a class of MaLR endogenous primate retrotransposons and suppresses the innate immune response to viral infection, at least in part through the activation of DEFB103, a human defensin that can inhibit muscle differentiation. These findings suggest specific mechanisms of FSHD pathology and identify biomarkers useful for disease diagnosis and progression.

As described in Example 7, siRNA knockdown of DUX4 confirmed that the activation of germline genes in FSHD muscle cells is due to the leaky expression of DUX4 in FSHD muscle cells. Therefore, agents that inhibit the activity of DUX4, either by eliminating its expression in the muscle cells, as done in vitro with an siRNA, or by introducing a dominant negative agent, such as the DUX4-s splice form are expected to be useful as therapeutic agents for treating and/or preventing FSHD, or symptoms related to FSHD.

As described in Example 8, DUX4 can prevent the innate immune response to viral infection in skeletal muscle cells, at least in part, through the transcriptional induction of DEFB103. As further described in Example 8, the inventors have discovered that DEFB103 suppresses the induction of skeletal muscle differentiation genes, beta-defensin 3 (DEFB103) inhibits muscle cell fusion and expression of myosin heavy chain in primary muscle.

### III. METHODS OF INHIBITING THE INNATE IMMUNE RESPONSE IN A MAMMALIAN SUBJECT

In one aspect, the invention provides a method of inhibiting the innate immune response in a mammalian subject in need thereof, comprising administering to the mammalian subject an agent capable of inducing, or increasing the level of DUX4-fl expression in a population of cells in the mammalian subject.

In some embodiments of this aspect of the invention, the method comprises administering a therapeutic agent that is capable of inducing or increasing the level of endogenous DUX4-fl. In some embodiments, the therapeutic agent is a demethylating agent, such as 5-azacytidine (decatibine).

In some embodiments of this aspect of the invention, the method comprises administering a therapeutic agent that blocks translation dependent nonsense mediated decay, such as cycloheximide, or an inhibitor of UPF, such as the inhibitor of UPF described in Sun et al., 1998), hereby incorporated herein by reference or an inhibitory nucleic acid that specifically binds a UPF gene, such as UPF1 siRNA, or an agent that inhibits the kinase dependent activation of UPF1.

In some embodiments of this aspect of the invention, the method comprises administering DUX4-fl polypeptide or a nucleic acid encoding DUX4-fl polypeptide in a composition formulated for in vivo delivery to a mammalian subject.

In some embodiments, the agent capable of increasing or inducing DUX4-fl expression is administered to the mammalian subject in an amount sufficient to inhibit or reduce the expression of at least one or more of the three primary sensors of viral RNA LGP2 (DHX58), IFIH1 (MDA5), and/or DDX58 (RIG-1)). In some embodiments, the agent is administered to the mammalian subject in an amount sufficient to inhibit the innate immune response to a viral infection in the mammalian subject.

In one embodiment, the invention provides a method of inhibiting the innate immune response in a mammalian subject by administering to the subject an agent capable of inducing, or increasing the level of DEFB103A (SEQ ID NO:49), and/or DEFB103B (SEQ ID NO:107) expression in a population of cells in the mammalian subject. In some embodiments, the method comprises administering the DEFB103A/B polypeptide (SEQ ID NO:178), or a nucleic acid molecule encoding the polypeptide (SEQ ID NO:49 or SEQ ID NO:107) to the mammalian subject.

In certain embodiments, the level of DEFB103A/B is increased by about, at least about, or at most about 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 400, 500, 600, 700, 800, 900, 1000% or more (or any range derivable therein) relative to the level in the absence of the agent.

The methods in accordance with this aspect of the invention can be used to suppress the immune response in order to induce immune tolerance, and may be used to treat a subject suffering from an autoimmune disease (e.g., Systemic Lupus Erythermatosis, Aicardi-Goutieres syndrome, or Multiple Sclerosis, or to prepare a subject for transplant, or treat a subject that is undergoing, or has undergone, an organ or tissue transplant.

In another aspect, the invention provides a method of suppressing or inhibiting the innate immune response in a mammalian subject in need thereof, comprising administering to the mammalian subject an agent capable of inducing, or increasing the level of DEFB103A and/or DEFB103B expression or activity in a population of cells in the mammalian subject. In some embodiments, the agent is capable of increasing endogenous DEFB103 mRNA expression in the cells. In some embodiments, the agent comprises a nucleic acid molecule (e.g., SEQ ID NO:49 and/or SEQ ID NO:107) encoding DEFB103 (SEQ ID NO:178). In some embodiments, the agent comprises the DEFB103 polypeptide (SEQ ID NO:178).

The method in accordance with this aspect of the invention can be used to treat a subject in need thereof selected from the group consisting of (i) a subject suffering from an autoimmune disease and (ii) a subject that is undergoing, or has undergone an organ or tissue transplant. In some embodiments, the autoimmune disease is selected from the group consisting of Systemic Lupus Erythermatosis, Aicardi-Goutieres syndrome and Multiple Sclerosis.

### IV. METHODS OF INCREASING OR MAINTAINING THE INNATE IMMUNE RESPONSE IN A MAMMALIAN SUBJECT

In another aspect, the invention provides a method of increasing or maintaining the innate immune response in a mammalian subject in need thereof. The method in accordance with this aspect of the invention comprises administering to the mammalian subject an agent capable of inhibiting, or suppressing the level of DUX4-fl expression, or an agent capable of inhibiting DUX4-fl mediated transcription activation in a population of cells in the mammalian subject.

In some embodiments of this aspect of the invention, the method comprises administering a therapeutic agent that is capable of inhibiting or suppressing the level of endogenous DUX4-fl. In some embodiments, the agent is capable of increasing chromatin mediated repression, such as an agent that inhibits histone demethylase LSD1 activity (e.g., paragline). In some embodiments, the agent enhances nonsense mediated decay and thereby enhances the degradation of DUX4 mRNA.

In some embodiments, the agent is capable of inhibiting DUX4-fl mediated transcriptional activation is an agent that interferes with DUX4-fl binding to one or more DUX4-fl consensus binding site(s) “TAAYBBAATCA” (SEQ ID NO: 166) that is present upstream of one or more DUX4-fl inducible genes. An exemplary agent for use in accordance with this embodiment is a DUX4-s polypeptide, or a nucleic acid encoding DUX4-s polypeptide. In some embodiments, the invention provides a pharmaceutical composition comprising a DUX4-s polypeptide or a nucleic acid encoding a DUX4-s polypeptide and a pharmaceutically acceptable carrier.

The methods in accordance with this aspect of the invention can be used to enhance the immune response, and can be used to treat a subject suffering from, or at risk for developing FSHD, or a subject suffering from, or at risk for developing myotonic dystrophy or Huntington's disease, or a subject suffering from cancer, or a subject that is infected with a pathogen, such as a viral infection (e.g., HIV).

In another aspect, the invention provides methods for increasing or maintaining the innate immune response in a mammalian subject in need thereof, comprising administering to the mammalian subject an agent capable of inhibiting, or suppressing the level of DEFB103A and/or DEFB103B expression in a population of cells in the mammalian subject.

In some embodiments, the agent capable of inhibiting DEFB103 expression is an agent that interferes with DUX4-fl binding to one or more DUX4-fl consensus binding site(s) “TAAYBBAATCA” (SEQ ID NO: 166) that is present upstream of DEFB103 (which is an DUX4-fl inducible gene). An exemplary agent for use in accordance with this embodiment is a DUX4-s polypeptide, or a nucleic acid encoding DUX4-s polypeptide. In some embodiments, the invention provides a pharmaceutical composition comprising a DUX4-s polypeptide or a nucleic acid encoding a DUX4-s polypeptide and a pharmaceutically acceptable carrier.

In another embodiment, the agent capable of inhibiting DEFB103 expression is a nucleic acid molecule (e.g., antisense, siRNA,) that specifically hybridizes to a nucleic acid encoding DEFB103, such as SEQ ID NO; 49 and/or SEQ ID NO:107.

In another embodiment, the agent capable of inhibiting DEFB103 activity specifically binds to DEFB103A polypeptide (SEQ ID NO:178), such as an antibody, or fragment thereof that is capable of inhibiting or blocking DEFB103 activity. In another embodiment, the agent capable of inhibiting DEFB103 activity is a small molecule inhibitor.

In some embodiments, the agent for use in this aspect of the invention is an agent that is capable of reducing the level of endogenous DUX4-fl. In some embodiments, the agent enhances translation dependent nonsense mediated decay.

The methods in accordance with this aspect of the invention can be used to maintain and/or enhance the innate immune response or treat or prevent a disease in need of modulation of DUX4 or its target gene such as DEFB103A or DEFB103B. In certain aspects, the methods can be used to restore the expression or activity of DUX4 or its target gene to a normal level. In certain aspects, the modulation may be inhition of the expression or activity of DUX4 or its target gene if DUX4 or its target gene is present at or is determined to have a higher expression or activity as compared to a normal control; in other aspects, the modulation may upregulation of the expression or activity of DUX4 or its target gene if the expression or activity in the subject is lower than a normal control.

For example, the methods can be used to treat a subject selected from the group consisting of (i) a subject suffering from, or at risk for developing FSHD, (ii) a subject suffering from or at risk for developing myotonic dystrophy, (iii) a subject suffering from or at risk for developing Huntington's disease, (iv) a subject suffering from cancer, (v) a subject suffering from an autoimmune disease, and (vi) a subject infected with a pathogen, such as a virus, such as HIV.

### V. METHODS OF MODULATING MYOGENESIS

In another aspect, the invention provides methods and compositions for modulating myogenesis in muscle cells.

In one embodiment, the invention provides a method of inhibiting myogenesis in muscle cells comprising contacting the cells with an agent capable of inducing or increasing the level of DEFB103. In some embodiments, the agent is capable of increasing endogenous DEFB103 mRNA expression in the cells. In some embodiments, the agent comprises a nucleic acid molecule encoding DEFB103. In some embodiments, the agent comprises DEFB103 polypeptide.

In another embodiment, the invention provides a method of promoting myogenesis in muscle cells comprising contacting the cells with an agent capable of inhibiting DEFB103 expression and/or DEFB103 activity. In some embodiments, the agent specifically binds to DEFB103A polypeptide or a nucleic acid encoding DEFB103A polypeptide. In some embodiments, the agent is capable of inhibiting DUX4-fl mediated transcriptional activation.

In another aspect, the invention provides a method of promoting or maintaining muscle differentiation in a mammalian subject in need thereof, comprising administering to the mammalian subject an agent capable of inhibiting, or suppressing the level of DEFB103 expression and/or inhibiting DEFB103 activity in a population of muscle cells in the mammalian subject. In some embodiments, the agent specifically binds to DEFB103A polypeptide or a nucleic acid encoding DEFB103A polypeptide. In some embodiments, the agent is capable of inhibiting DUX4-fl mediated transcriptional activation. In some embodiments, the subject in need thereof is selected from the group consisting of (i) a subject suffering from, or at risk for developing FSHD, (ii) a subject suffering from or at risk for developing myotonic dystrophy, (iii) a subject suffering from or at risk for developing Huntington's disease, (iv) a subject suffering from or at risk for developing muscular dystrophy, (v) a subject suffering from or at risk for developing sarcopenia.

### VI. METHODS OF INDUCING TESTIS-EXPRESSED GENES IN NON-TESTIS CELL TYPES

In another aspect, the invention provides a method of inducing one or more testis expressed genes (e.g., cancer testis antigens) in a non-testis cell type comprising contacting the non-testis cell type with an agent capable of inducing, or increasing the level of DUX4-fl expression in a population of cells.

In some embodiments, the agent for use in this aspect of the invention is an agent that is capable of inducing or increasing the level of endogenous DUX4-fl. In some embodiments, the agent is a demethylating agent, such as 5-azacytidine (decatibine). In some embodiments, the agent blocks translation dependent nonsense mediated decay, such as cycloheximide.

In some embodiments, the agent is a DUX4-fl polypeptide or a nucleic acid encoding DUX4-fl polypeptide. In some embodiments, the invention provides a pharmaceutical composition comprising a DUX4-fl polypeptide or a nucleic acid encoding a DUX4-fl polypeptide and a pharmaceutically acceptable carrier.

The methods of this aspect of the invention may be carried out by contacting any type of non-testis cell type, including for example, skeletal muscle, cancer cells, and dendritic cells. The methods can be used in vitro or in vivo to enhance the immune response to the induced proteins for the purpose of expanding or activating T-cell populations, such as for anti-cancer therapies. This can be applied to immune therapy to stimulate T-cells to a cancer, or as a vaccine therapy to induce immunity to these antigens.

### VII. PHARMACEUTICAL PREPARATIONS, CARRIERS AND DELIVERY VEHICLES

In general, the agents for use in the methods of the present invention, are suitably contained in a pharmaceutically acceptable carrier. The carrier is non-toxic, biocompatible and is selected so as not to detrimentally affect the biological activity of the agent. The agents of the invention may be formulated into preparations for local delivery (i.e. to a specific location of the body, such as skeletal muscle or other tissue) or systemic delivery, in solid, semi-solid, gel, liquid or gaseous forms such as tablets, capsules, powders, granules, ointments, solutions, depositories, inhalants and injections allowing for oral, parenteral or surgical administration. The invention also contemplates local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting example, microparticles, microspheres, nanospheres or nanoparticles composed of proteins, liposomes, carbohydrates, synthetic organic compounds, inorganic compounds, polymeric or copolymeric hydrogels and polymeric micelles.

The actual dosage amount of a composition of the present invention administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound, such as an isolated DUX4-fl polypeptide or DEFB103 polypeptide or their expression constructs, inhibitory antibodies or inhibitory nucleic acids. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 μg/kg/body weight to about 100 mg/kg/body weight, about 5 microgram/kg/body weight to about 500 milligram/kg/body weight, etc., can be administered. A gene expression inhibitor may be administered in a dose of 1, 2, 3, 4, 5, 6, 7, 8, 9, 10, 15, 20, 25, 30, 40, 50, 60, 70, 80, 90, 100 or more μg of nucleic acid per dose. Each dose may be in a volume of 1, 10, 50, 100, 200, 500, 1000 or more μl or ml.

Solutions of therapeutic compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

The therapeutic compositions of the present invention may include classic pharmaceutical preparations. Administration of therapeutic compositions according to the present invention will be via any common route so long as the target tissue is available via that route. This includes oral, nasal, buccal, rectal, vaginal or topical. Topical administration may be particularly advantageous for the treatment of skin cancers, to prevent chemotherapy-induced alopecia or other dermal hyperproliferative disorder. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol is between about 0.01 ml and 0.5 ml.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses discussed above in association with its administration, i.e., the appropriate route and treatment regimen. The quantity to be administered, both according to number of treatments and unit dose, depends on the protection or effect desired.

Precise amounts of the therapeutic composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### VIII. BIOMARKERS USEFUL FOR ASSESSING THE PRESENCE OR RISK OF DEVELOPING FSHD IN A MAMMALIAN SUBJECT

In one aspect, the invention provides a plurality of biomarkers useful for assessing the presence or risk of developing FSHD in a mammalian subject, wherein the FSHD biomarkers comprise a gene product of a DUX-4-fl induced gene, as set forth in TABLE 1 or TABLE 2. As described in Example 1, the inventors have determined that DUX4-fl (SEQ ID NO:110), encoded by DUX4-fl splice variant 1 cDNA (introns 1 and 2 are spliced) (Genbank No. HQ266760): (SEQ ID NO:108), or DUX4-fl splice variant 2 cDNA (intron 2 is spliced) (Genbank No. HQ266761): (SEQ ID NO:109), activates the expression of germline genes set forth in TABLE 1 and TABLE 2, referred to as “FSHD biomarkers.” The FSHD markers in TABLE 2 are a subset of the markers in TABLE 1. The gene products expressed from the gene markers listed in TABLES 1 and 2, or the polypeptides encoded by the gene markers, may be detected in accordance with the methods described herein for assessing the presence or risk of developing FSHD in a mammalian subject.

In some embodiments, the invention provides an isolated polynucleotide probe for detecting an FSHD biomarker, or a polynucleotide primer for amplifying at least a portion of an FSHD biomarker, wherein the nucleic acid probe or primer has a length of from at least 10 nucleotides to 200 nucleotides or longer, and specifically hybridizes to the nucleic acid sequence of at least one FSHD biomarker set forth in TABLE 1 or TABLE 2.

In some embodiments, the invention provides an isolated population of polynucleotide probes comprising a plurality of polynucleotides each complementary and hybridizable to a sequence of at least two or more (i.e. at least 3, 4, 5, 10, 15, 20, or more) different FSHD biomarkers selected from any one of TABLE 1 or TABLE 2. In some embodiments, the isolated population of polynucleotide probes are attached to a diagnostic tool for diagnosing or predicting the risk of developing FSHD in a human subject. In some embodiments, the polynucleotide probes are immobilized on a solid support, such as, for example a microarray. In one embodiment, the isolated population of polynucleotide probes comprise PCR primers for amplifying a portion of one or more FSHD biomarkers selected from TABLES 1 or 2. PCR primers are preferably chosen based on the sequence of the marker that will result in amplification of specific fragments of the marker gene. Computer programs that are well known in the art are useful in the design of primes with the required specificity and optimal amplification properties, such as Oligo version 5.0 (National Biosciences). PCR methods are well known in the art, and are described, for example, in Innis et al. (1990).

In another aspect, the invention provides one or more isolated antibodies that specifically bind to one or more FSHD polypeptide biomarker(s) encoded by the nucleic acid sequences set forth in TABLE 1 or TABLE 2. In some embodiments, the isolated antibodies further comprise a detectable label for use in a diagnostic assay. In some embodiments, one or more antibodies are bound to the surface of diagnostic tool (e.g., an immunoassay plate, a bead or a resin) for diagnosing or predicting the risk of developing FSHD in a human subject. The antibodies capable of binding to the polypeptides encoded by the one or more FSHD biomarkers can be polyclonal or monoclonal.

In certain embodiments, a diagnosis or risk assessment of FSHD can be made by analyzing the presence or amount of one or more FSHD biomarker polypeptide(s), by a variety of methods, including methods described herein, and also generally methods comprising spectroscopy, colorimetry, electrophoresis, isoelectric focusing, immunoprecipitations, and immunofluorescence, and immunoassays (e.g., David et al., U.S. Pat. No. 4,376,110) such as, for example immunoblotting (see also Current Protocols in Molecular Biology, particularly chapter 10).

Both quantitative and qualitative increases of the FSHD biomarker polypeptides are encompassed by the present invention. For example, in a particular embodiment, an antibody capable of binding to the polypeptide, preferably an antibody with a detectable label or an antibody that can be detected by a secondary antibody, can be used.

Antibodies can be polyclonal or monoclonal, and may be generated according to well known methods in the art, for example, monoclonal antibodies can be prepared, for example, using hybridoma technology (Köhler and Milstein, 1975).

An antibody in certain aspects of the present invention can be an intact immunoglobulin derived from natural sources or from recombinant sources, and can be immunoreactive portions of an intact immunoglobulin (including, for example, an antibody fragment and a single chain antibody). An antibody is typically a tetramer of immunoglobulin molecules. An antibody of the present invention can be prepared by a variety of methods (Coligan et al., 1991). For example, cells expressing a polypeptide of the present invention are administered to an animal to induce the production of sera containing polyclonal antibodies.

In particular aspects, a preparation of the secreted protein is prepared and purified to render it substantially free of natural contaminants. Such a preparation is then introduced into an animal in order to produce polyclonal antisera of greater specific activity.

In particular embodiments, an antibody of the present invention is a monoclonal antibody (mAb), or protein binding fragment thereof. Such monoclonal antibody can be prepared, for example, using hybridoma technology (Köhler and Milstein, 1975; Köhler and Milstein, 1976; Köhler et al., 1976; Monoclonal Antibodies and T-Cell Hybridomas, Elsevier, N.Y., pp. 563-681, 1981). In general, such methods involve immunizing an animal (e.g., a mouse) with polypeptide or with a secreted polypeptide-expressing cell. The splenocytes of, for example, such mice following the methods described above are extracted and fused with a suitable myeloma cell-line. The hybridoma cells obtained through such a selection are then assayed to identify clones that secrete antibodies capable of binding the polypeptide. An intact antibody, or a fragment thereof (e.g., Fab or F (ab′) 2) can be used. The term “labeled” with regard to the probe or antibody, is intended to encompass direct labeling of the antibody by coupling (i.e., physically linking) a detectable substance to the antibody, as well as indirect labeling of the antibody by reactivity with another reagent that is directly labeled or indirectly labeled. Examples of direct and indirect labels include, for example, a fluorescent moiety, an enzyme, a chromophoric moiety, a radioactive atom, a biotin tag, or a colorimetric tag. Some examples of a fluorescent moiety include rhodamine, fluorescein, etc. Some examples of enzymes include, horseradish peroxidase, glucose oxidase, glucose-6-phosphate dehydrogenase, alkaline phosphatase, beta-galactosidase, urease, luciferase, etc. Some examples of radioactive atoms are ³²P, ¹²⁵I, ³H, etc.

In some methods, a patient is identified as having one or more biomarkers indicative of FSHD or of being at risk for FSHD. In further embodiments, there is a step of reporting the patient as having one or more biomarkers indicative of FSHD or of being at risk for FSHD. Alternatively, in other embodiments there is a step of reporting the presence or absence of a biomarker or reporting the level of the biomarker. Additional embodiments include reporting to the subject or patient or to a treating clinician the results of any analysis or determination. Such reporting can involve an electronic or physical document.

Methods may also involve comparing a level of a biomarker to a control or reference level that reflects either the level of a patient who has FSHD or is at risk of FSHD or to a patient who does not have FSHD or who is not at risk for FSHD.

In additional embodiments, a patient identified as having or being at risk for FSHD may be treated accordingly. Further embodiments may involve knowing that a patient or subject is at risk for FSHD based on an analysis or determination discussed herein and subsequently treating or counseling the patient accordingly. A clinician may discuss lifestyle options to minimize muscle damage, career counseling and/or genetic counseling. These things may occur after a subject or patient is identified as having or being at risk for FSHD.

In another aspect, the invention provides a kit comprising one or more detection reagents for detecting one or more FSHD biomarkers set forth in TABLE 1 or TABLE 2 for use in an assay to determine the presence or risk of FSHD in a biological sample obtained from a mammalian subject. Reagents that are suited for obtaining a sample from an individual may be included in a kit of the invention, such as a syringe, collection vial, needle, or other instruments necessary to take a biopsy or other relevant sample. The kits may comprise a suitably aliquoted composition and/or additional agent compositions of the present invention, whether labeled or unlabeled, as may be used to prepare a standard curve for a detection assay. The components of the kit may be packaged in combination or alone in the same or in separate containers, depending on, for example, cross-reactivity or stability, and can also be supplied in solid, liquid, lyophilized, or other applicable form. The container means of the kits will generally include, for example, at least one vial, test tube, flask, bottle, syringe or other container means, into which a component may be placed, and preferably, suitably aliquoted. Where there is more than one component in the kit, the kit can contain a second, third or other additional container into which the additional components may be contained. However, various combinations of components may be comprised in a vial. The kits of the present invention also will typically include a means for containing the composition, additional agent and any other reagent containers in close confinement for commercial sale. Such containers may include, for example, injection or blow molded plastic containers into which the desired vials are retained.

### IX. METHODS OF DETERMINING THE PRESENCE OF FSHD, OR RISK OF DEVELOPING FSHD IN A MAMMALIAN SUBJECT

In one aspect, the invention provides a method of determining the presence of, or risk of developing, Facioscapulohumeral dystrophy (FSHD) in a mammalian subject. The method in accordance with this aspect comprises: (a) determining the presence or amount of at least one FSHD biomarker in a biological test sample obtained from a mammalian subject, wherein the at least one FSHD biomarker comprises a gene product of a DUX-4-fl induced gene; and (b) comparing the presence or amount of the biomarker determined in step (a) with a reference standard or control sample, wherein an increase in the presence or amount of the FSHD biomarker determined in the test sample in comparison to the reference standard or control sample is indicative of the presence of FSHD, or increased risk of developing FSHD, in the mammalian subject.

In some embodiments, the DUX4-fl induced gene is expressed from a promoter comprising at least one or more DUX-4 responsive elements comprising the consensus sequence “TAAYBBAATCA” (SEQ ID NO:166). In some embodiments, the at least one DUX4-fl induced gene product (mRNA or polypeptide) is expressed at an increased level of at least 2-fold or greater (i.e. at least 3-fold, at least 5-fold, at least 8-fold, at least 10-fold, at least 16 fold or greater) in FSHD skeletal muscle as compared to normal control skeletal muscle. In some embodiments, the method comprises determining the presence or amount of at least one or more FSHD biomarkers selected from TABLE 1 or TABLE 2 in a biological test sample by contacting the sample with a detection reagent (e.g., a PCR primer or antibody) that specifically detects a nucleic acid (e.g., mRNA) or polypeptide expressed from, or derived from, the FSHD biomarker.

In some embodiments, the method comprises determining the presence or amount of a nucleic acid or polypeptide expressed from, or derived from, at least one DUX4-fl induced gene selected from the group consisting of: TRIM43 (SEQ ID NO:62), TRIM48 (SEQ ID NO:23), KHDC1 (SEQ ID NO:21), MBD3L2 (SEQ ID NO:29), PRAMEF1 (SEQ ID NO:16), PRAMEF2 (SEQ ID NO:28), ZSCAN4 (SEQ ID NO:5), RFPL2 (SEQ ID NO:36), CCNA1 (SEQ ID NO:31), DEFB103A (SEQ ID NO:49), and DEFB103B (SEQ ID NO:107).

In some embodiments, the method comprises performing quantitative RT-PCR on the biological sample with reagents that specifically hybridize to the mRNA expressed from the DUX4-fl induced gene. In some embodiments, the method comprises contacting the biological test sample with an antibody that specifically binds to the at least one biomarker. In some embodiments, the method comprises analyzing the biological sample with mass spectrometry to detect the presence or amount of the at least one biomarker.

In some embodiments, the method comprises determining the presence or amount of two or more different FSHD biomarkers (i.e., at least 3, 4, 5, 10, 15, 20, or more) selected from any one of TABLE 1 or TABLE 2 in the biological test sample.

In some embodiments, the biological test sample is obtained from a mammalian living fetus, such as a living human fetus. In some embodiments, the biological test sample is obtained from a subject suspected of having FSHD. In some embodiments, the biological test sample is obtained from a subject with a family member diagnosed with FSHD. In some embodiments, the biological test sample is obtained from a subject known to have FSHD, for example, in an embodiment in which the method is used for monitoring disease activity or progression, or response to therapy in a clinical trial or during therapeutic intervention.

In some embodiments, the biological test sample is selected from the group consisting of a muscle biopsy, blood, plasma, serum, urine, saliva, tears,

In accordance with the practice of various embodiments of the invention, polynucleotide molecules are extracted from a biological sample taken from a mammalian subject. The sample may be collected in any clinically acceptable manner, but must be collected such that marker-derived polynucleotides (i.e., RNA) are preserved and/or marker-derived polypeptides are preserved. In some embodiments, mRNA or nucleic acids derived therefrom (i.e., cDNA or amplified DNA) are preferably labeled distinguishably from standard or control polynucleotide molecules, and both are simultaneously or independently hybridized to a nucleic acid array, such as a microarray comprising some or all of the markers or marker sets or subsets described above. Alternatively, mRNA or nucleic acids derived therefrom may be labeled with the same label as the standard or control polynucleotide molecules, wherein the intensity of hybridization of each at a particular probe is compared. Methods for preparing total and poly(A)+RNA are well known and are described generally in Sambrook et al. (1989) and Ausubel et al. (1994).

In accordance with the methods of the invention, the presence or amount of the at least one FSHD biomarker in the test biological sample is compared with a reference standard or control sample, wherein an increase in the presence or amount of the FSHD biomarker determined in the test sample in comparison to the reference standard or control sample is indicative of the presence of FSHD, or increased risk of developing FSHD, or predict disease onset in the mammalian subject. In some embodiments, an increase in the presence or amount of the FSHD biomarker provides a clinical diagnosis of FSHD. In some embodiments, an increase in the presence or amount of the FSHD biomarker is indicative of disease progression. In some embodiments, a decrease in the amount of the FSHD biomarker is indicative of improvement of pathology in response to a therapeutic agent.

In one embodiment, the reference standard is the level of the one or more FSHD biomarkers measured in one or more biological sample(s) obtained from healthy control subjects known not to have FSHD. One or more, including 2, 3, 4, 5, 10 or more healthy individuals can be used to generate a reference standard for use in the methods. When multiple individuals are used to generate a reference standard for a particular FSHD biomarker, the biomarker levels determined from the individuals can be averaged to create a single reference standard value.

In another embodiment, the reference standard is an established threshold level. In one embodiment, the methods comprises the use of a control sample which may be obtained from a healthy subject.

In some embodiments, a determination is made that the mammalian subject from which the test sample was obtained has FSHD, or has an increased risk of developing FSDH, when the FSDH biomarker is found to be expressed at an increased level of at least 2-fold or greater (i.e. at least 3-fold, at least 4-fold, at least 5-fold, at least 8-fold, at least 10-fold, at least 12-fold, at least 16-fold or greater) in the test biological sample (e.g., skeletal muscle) as compared to the control or reference standard (e.g., normal skeletal muscle).

In another aspect, the invention provides a nucleic acid molecule comprising an expression cassette comprising a promoter operationally linked to a reporter gene or selectable marker, wherein the promoter contains at least one DUX4-responsive element (i.e., one, two, three, four or more) comprising the consensus sequence “TAAYBBAATCA” (SEQ ID NO:166). The reporter gene may be any suitable reporter gene used in the art. Non-limiting Examples of such reporter genes include chloramphenicol acetyl transferase (CAT) or luciferase. Non-limiting examples of suitable promoters include viral promoters such as a CMV or SV40 promoter. In some embodiments, the expression cassette is contained on an expression vector. In some embodiments, the expression cassette is present in a mammalian cell.

In another aspect, the invention provides a method of detecting the presence of DUX4-fl protein in a cell sample comprising introducing a nucleic acid molecule comprising an expression cassette comprising a promoter operationally linked to a reporter gene or selectable marker, wherein the promoter contains at least one DUX4-responsive element comprising the consensus sequence “TAAYBBAATCA” (SEQ ID NO:166), and assaying the cell for expression of the reporter gene, or selecting for growth under conditions requiring expression of the selectable marker, wherein expression of the reporter gene or growth under conditions requiring expression of the selectable marker indicate the presence of DUX4-fl protein.

In another aspect, the invention provides a method of identifying an inhibitor of DUX4-fl induced expression. The methods according to this aspect comprise: (a) contacting a cell containing: (i) a nucleic acid molecule comprising an expression cassette comprising a promoter operationally linked to a reporter gene or selectable marker, wherein the promoter contains at least one DUX4-responsive element comprising the consensus sequence “TAAYBBAATCA” (SEQ ID NO:166), and (ii) DUX4-fl polypeptide, with a candidate inhibitory agent; and (b) determining whether the cell expresses the reporter gene or selectable marker in the presence and absence of the candidate inhibitory agent, wherein the absence of expression of the reporter gene or selectable marker in the presence of the inhibitory agent indicates that the agent is an inhibitor of DUX4-fl induced expression. Candidate inhibitor compounds which may be used in accordance with this aspect of the invention may be natural or synthetic chemical compounds used in drug screening programmes (i.e. small molecules), or may be polypeptides (i.e. inhibitory peptides or antibodies).

### X. MODULATION OF GENE EXPRESSION OR ACTIVITY

Aspects of the invention include modulating expression or activity of genes, such as DUX4, or its target genes, particularly DEFB103A or DEFB103B, in cells, tissues, or organs of a subject. Depending on the particular treatment purposes, the modulation may include inhibiting gene expression or activity by introducing inhibitory peptides or inhibitory nucleic acids to the subject, or increasing gene expression or activity by introducing isolated polypeptides or exogenous expression constructs or by increasing the endogenous gene expression.

A. Inhibition of Gene Expression or Activity

1. Inhibitory Peptides

In certain aspects, methods and compositions may be provided to inhibit the activity of particular polypeptides or peptides in a cell, for example, by molecules that specifically binds DUX4-fl or peptides encoded by its target genes, or specifically binds DUX4-responsive elements on its target genes but has reduced or no activity for regulating target genes as compared to native DUX4-fl (such as a dominant negative mutant). Such molecules may be an antibody, an isolated polypeptide or peptide, a synthetic peptide or a small molecule. The antibody may be selected from the group consisting of a chimeric antibody, an affinity matured antibody, a polyclonal antibody, a monoclonal antibody or a humanized antibody, and a human antibody. In a particular example, the antibody is a monoclonal antibody or a humanized antibody. In another example, the antibody is a polyclonal antibody. For example, a dominant negative mutant for DUX4-fl may be a DUX4 truncated form that lacks the transcriptional activation domain (such as DUX4-s)

In one embodiment, the antibody is a chimeric antibody, for example, an antibody comprising antigen binding sequences from a non-human donor grafted to a heterologous non-human, human or humanized sequence (e.g., framework and/or constant domain sequences). In one embodiment, the non-human donor is a mouse. In one embodiment, an antigen binding sequence is synthetic, e.g., obtained by mutagenesis (e.g., phage display screening, etc.). In one embodiment, a chimeric antibody of the invention has murine V regions and human C region. In one embodiment, the murine light chain V region is fused to a human kappa light chain. In one embodiment, the murine heavy chain V region is fused to a human IgG1 C region.

Examples of antibody fragments suitable for the present invention include, without limitation: (i) the Fab fragment, consisting of VL, VH, CL and CH1 domains; (ii) the “Fd” fragment consisting of the VH and CH1 domains; (iii) the “Fv” fragment consisting of the VL and VH domains of a single antibody; (iv) the “dAb” fragment, which consists of a VH domain; (v) isolated CDR regions; (vi) F(ab′)2 fragments, a bivalent fragment comprising two linked Fab fragments; (vii) single chain Fv molecules (“scFv”), wherein a VH domain and a VL domain are linked by a peptide linker which allows the two domains to associate to form a binding domain; (viii) bi-specific single chain Fv dimers (see U.S. Pat. No. 5,091,513) and (ix) diabodies, multivalent or multispecific fragments constructed by gene fusion (US Patent App. Pub. 20050214860). Fv, scFv or diabody molecules may be stabilized by the incorporation of disulphide bridges linking the VH and VL domains. Minibodies comprising a scFv joined to a CH3 domain may also be made (Hu et al, 1996).

Methods have been developed to replace light and heavy chain constant domains of the monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent and human amino acid sequences. In “humanized” monoclonal antibodies, only the hypervariable CDR is derived from mouse monoclonal antibodies, and the framework regions are derived from human amino acid sequences. It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

It is possible to create engineered antibodies, using monoclonal and other antibodies and recombinant DNA technology to produce other antibodies or chimeric molecules which retain the antigen or epitope specificity of the original antibody, i.e., the molecule has a binding domain. Such techniques may involve introducing DNA encoding the immunoglobulin variable region or the CDRs of an antibody to the genetic material for the framework regions, constant regions, or constant regions plus framework regions, of a different antibody. See, for instance, U.S. Pat. Nos. 5,091,513, and 6,881,557, which are incorporated herein by this reference.

2. Inhibitory Nucleic Acids

In certain aspects of the present invention, inhibitors for DUX4-fl and its target genes (such as DEFB103A or DEFB103B) may be used for treating a subject. For example, an DUX4-specific inhibitory nucleic acid or an inhibitory nucleic acid for UPF1 a may be used.

Examples of an inhibitory nucleic acid include but are not limited to siRNA (small interfering RNA), short hairpin RNA (shRNA), double-stranded RNA, an antisense oligonucleotide, a ribozyme and a nucleic acid encoding thereof.

In another embodiment, the inhibitory nucleic acid such as an siRNA molecule of a DUX4-fl gene or a related gene (as a template) has a sequence that is at least 75, 80, 81, 82, 83, 84, 85, 86, 87, 88, 89, 90, 91, 92, 93, 94, 95, 96, 97, 98, 99 or 100% identity, preferably 95%, 99%, or 100% identity, to at least 10, 20, 50, 100, or 200 contiguous nucleotides of the nucleic acid sequences of a template. Without undue experimentation and using the disclosure of this invention, it is understood that additional siRNAs that modulate a template gene's expression can be designed and used to practice the methods of the invention.

An inhibitory nucleic acid may inhibit the transcription of a gene or prevent the translation of a gene transcript in a cell. An inhibitory nucleic acid may be from 10, 11, 12, 13, 14, 15, 16, 17, 18, 19, 20, 30, 40, 50, 60, 70, 80, 100, 200, 300, 400, 500, 600, 700, 800, 900, to 1000 nucleotides long, and in certain embodiments from 18 to 100 nucleotides long.

Particularly, an inhibitory nucleic acid or analog may be capable of decreasing the expression of a target gene, such as DUX4 or its target gene such as DEFB103A/B or its upstream regulator UPF1, by at least 10%, 20%, 30%, or 40%, more particularly by at least 50%, 60%, or 70%, and most particularly by at least 75%, 80%, 90%, 95% or more or any ranges in between the foregoing.

Inhibitory nucleic acids are well known in the art. For example, siRNA and double-stranded RNA have been described in U.S. Pat. Nos. 6,506,559 and 6,573,099, as well as in U.S. Patent Publications 2003/0051263, 2003/0055020, 2004/0265839, 2002/0168707, 2003/0159161, and 2004/0064842, all of which are herein incorporated by reference in their entirety.

For example, the inhibitory nucleic acid may be siRNA. siRNA can be obtained from commercial sources, natural sources, or can be synthesized using any of a number of techniques well-known to those of ordinary skill in the art. For example, commercial sources of predesigned siRNA include Invitrogen's Stealth™ Select technology (Carlsbad, Calif.), Ambion® (Austin, Tex.), and Qiagen® (Valencia, Calif.). An inhibitory nucleic acid that can be applied in the compositions and methods of the present invention may be any nucleic acid sequence that has been found by any source to be a validated down-regulator of a corresponding gene.

Certain embodiments of the present invention pertain to methods of inhibiting expression of DUX4-fl and its target genes in a cell by introduction of inhibitory nucleic acids or analogs into the cell. Introduction of nucleic acids or analogs into cells can be achieved by methods known in the art, including for example, microinjection, electroporation, or transfection of a vector comprising a nucleic acid from which the siRNA can be transcribed. Alternatively, an inhibitory nucleic acid or analog can be directly introduced into a cell in a form that is capable of binding to target desired mRNA transcripts. To increase durability and membrane-permeability the inhibitory nucleic acid or analog may be combined or modified with liposomes, poly-L-lysine, lipids, cholesterol, lipofectine or derivatives thereof. In a particular aspect, the inhibitory nucleic acid analog may be an antisense morpholino molecule.

3. Other Inhibitory Agents

In some embodiments of this aspect of the invention, the method comprises administering a therapeutic agent that is capable of inhibiting or suppressing the level of endogenous DUX4-fl.

In some embodiments, the agent is capable of increasing chromatin mediated repression, such as an agent that inhibits histone demethylase LSD1 activity (e.g., pargyline). Pargyline (Eutonyl; N-Benzyl-N-methylprop-2-yn-1-amine) is an irreversible monoamine oxidase B (MAO-B) inhibitor.

In some embodiments, the agent enhances nonsense mediated decay and thereby enhances the degradation of DUX4 mRNA.

B. Enhancement of Gene Expression or Activity

1. Enhancement of Endogenous Expression

In some embodiments of this aspect of the invention, the method comprises administering a therapeutic agent that blocks or reduces translation-dependent nonsense mediated decay, such as cycloheximide (a protein synthesis inhibitor), or an inhibitor of UPF, such as the inhibitor of UPF described in Sun et al. (1998), hereby incorporated herein by reference, or an agent that inhibits the kinase dependent activation of UPF1.

2. Isolated Polypeptides

In certain aspects, the invention is directed to a pharmaceutical composition comprising DUX4-fl polypeptide or peptides encoded by DUX4-fl target genes, such as DEFB103A or DEFB103B, or a peptide or polypeptide derived there from. It is contemplated that the compositions and methods disclosed herein may be utilized to express all or part of sequences selected from the group consisting of SEQ ID NOs:1-107 (TALE 2) and derivatives thereof. The structure of the various polypeptides or peptides can be modeled or resolved by computer modeling, NMR, or x-ray crystallography. Such structures may be used to engineer derivatives of a particular native protein.

The following is a discussion based upon changing of the amino acids of a native polypeptide described herein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and in its underlying DNA or RNA coding sequence, and nevertheless produce a protein with like properties. It is thus contemplated by the inventors that various changes may be made in the DNA or RNA sequences of genes or coding regions without appreciable loss of their biological utility or activity, as discussed herein.

Various types of expression vectors are known in the art that can be used for the production of protein or peptide products. For example, following transfection with a expression vector comprising a coding sequence selected from the group consisting of SEQ ID NOs:1-107 to a cell in culture, e.g., a primary mammalian cell, a recombinant protein product may be prepared in various ways. A host cell strain may be chosen that modulates the expression of the inserted sequences, or that modifies and processes the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed. In order for the cells to be kept viable while in vitro and in contact with the expression construct, it is necessary to ensure that the cells maintain contact with the correct ratio of oxygen and carbon dioxide and nutrients but are protected from microbial contamination.

Certain aspects of the present invention concern the purification, and in particular embodiments, the substantial purification, of an encoded protein or peptide. The term “isolated or purified protein or peptide” as used herein, is intended to refer to a composition, isolatable from other components, wherein the protein or peptide is purified to any degree relative to its naturally obtainable state. A isolated or purified protein or peptide therefore also refers to a protein or peptide, free from the environment in which it may naturally occur.

3. Exogenous Expression Constructs

Aspects of the invention include introducing into a cell with an expression construct comprising at least a particular peptide, such as DUX4-fl polypeptide or peptides encoded by DUX4-fl target genes, such as DEFB103A or DEFB103B. In other aspects, expression construct may include one or more additional nucleic acid sequences, such as additional reporters, additional coding regions, or additional promoters.

In certain embodiments of the present invention, transfer of an expression construct into a cell is accomplished using a viral vector. Techniques using “viral vectors” are well-known in the art. A viral vector is meant to include those constructs containing viral sequences sufficient to (a) support packaging of the expression cassette and (b) to ultimately express a recombinant gene construct that has been cloned therein.

Several non-viral methods for the transfer of nucleic acids into cells also are contemplated by certain aspects of the present invention. These include calcium phosphate precipitation (Graham and Van Der Eb, 1973; Chen and Okayama, 1987; Rippe et al, 1990) DEAE-dextran (Gopal, 1985), electroporation (Tur-Kaspa et al, 1986; Potter et al, 1984), nucleofection (Trompeter et al, 2003), direct microinjection (Harland and Weintraub, 1985), DNA-loaded liposomes (Nicolau and Sene, 1982; Fraley et al, 1979) and lipofectamine-DNA complexes, polyamino acids, cell sonication (Fechheimer et al, 1987), gene bombardment using high velocity microprojectiles (Yang et al, 1990), polycations (Boussif et al, 1995) and receptor-mediated transfection (Wu and Wu, 1987; Wu and Wu, 1988). Some of these techniques may be successfully adapted for in vivo or ex vivo use. A person of ordinary skill in the art would be familiar with the techniques pertaining to use of nonviral vectors, and would understand that other types of nonviral vectors than those disclosed herein are contemplated by the present invention. In a further embodiment of the invention, the expression cassette may be entrapped in a liposome or lipid formulation. Liposomes are vesicular structures characterized by a phospholipid bilayer membrane and an inner aqueous medium. Multilamellar liposomes have multiple lipid layers separated by aqueous medium. Also contemplated is a gene construct complexed with Lipofectamine (Gibco BRL). One of ordinary skill in the art would be familiar with techniques utilizing liposomes and lipid formulations.

### XI. DISEASES

Diseases to be prevented, treated or diagnosed can be any disease that affects a subject that would be amenable to therapy or prevention through administration of a composition or a therapeutic agent as described herein. For example, the disease may be a disease amenable to the therapy for modulation of DUX4-fl expression or activity or its target genes such as DEFB103A/B. In particular examples, there may provided methods and compositions involving inhibition or suppression of DUX4-fl or its target gene DEFB103A/B for treating muscular dystrophy or myotonic dystrophy.

Examples include muscular diseases, cancer, infections, diabetes, cardiovascular disease, neurological disease, neurodegenerative disease, genetic disease, liver disease, infection, trauma, toxicity, or immunological disease.

A. Muscular Dystrophy

According to an embodiment of the invention, the methods described herein are useful in inhibiting the development of and/or treating muscular dystrophy or myotonic dystrophy. In a specific embodiment, treatment is by inhibiting or reducing the expression of DUX4-fl or DEFB103A/B.

Muscular dystrophy (MD) is a group of muscle diseases that weaken the musculoskeletal system and hamper locomotion. Muscular dystrophies are characterized by progressive skeletal muscle weakness, defects in muscle proteins, and the death of muscle cells and tissue.

It soon became evident that the disease had more than one form. In addition to Duchenne muscular dystrophy, the other major forms are Becker, limb-girdle, congenital, facioscapulohumeral, myotonic, oculopharyngeal, distal, and Emery-Dreifuss muscular dystrophy. These diseases predominately affect males, although females may be carriers of the disease gene. Most types of MD are multi-system disorders with manifestations in body systems including the heart, gastrointestinal system, nervous system, endocrine glands, eyes and brain.

Apart from the nine major types of muscular dystrophy listed above, several MD-like conditions have also been identified. Normal intellectual, behavioral, bowel and sexual function is noticed in individuals with other forms of MD and MD-like conditions. MD-affected individuals with susceptible intellectual impairment are diagnosed through molecular characteristics but not through problems associated with disability. However, a third of patients who are severely affected with MD may have cognitive impairment, behavioral, vision and speech problems.

Myotonic dystrophy (dystrophia myotonica, myotonia atrophica) is a chronic, slowly progressing, highly variable, inherited multisystemic disease. It is characterized by wasting of the muscles (muscular dystrophy), cataracts, heart conduction defects, endocrine changes, and myotonia. Two types of myotonic dystrophy exist.

Myotonic dystrophy type 1 (DM1), also called Steinert disease, has a severe congenital form and a milder childhood-onset form. Myotonic dystrophy type 2 (DM2), also called proximal myotonic myopathy (PROMM) or adult-onset form, is rarer than DM1 and generally manifests with milder signs and symptoms. Myotonic dystrophy can occur in patients of any age. Both forms of the disease display an autosomal dominant pattern of inheritance.

B. Cancer

The present invention may be used to treat a disease, such as cancer. For example, a pharmaceutical preparation may be delivered to treat a cancer. The cancer may be a solid tumor, metastatic cancer, or non-metastatic cancer. In certain embodiments, the cancer may originate in the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In certain embodiments, the cancer is human ovarian cancer. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Nonetheless, it is also recognized that the present invention may also be used to treat a non-cancerous disease (e.g., a fungal infection, a bacterial infection, a viral infection, and/or a neurodegenerative disease).

C. AIDS or HIV-1 Infection

According to an embodiment of the invention, the methods described herein are useful in inhibiting the development of and/or treating AIDS or HIV-1 infections. In a specific embodiment, treatment is by inhibiting or reducing the expression of DUX4-fl or DEFB103A/B.

In accordance with another embodiment, the methods of this invention can be applied in conjunction with, or supplementary to, the customary treatments of AIDS or HIV-1 infection. Historically, the recognized treatment for HIV-1 infection is nucleoside analogs, inhibitors of HIV-1 reverse transcriptase (RT). Intervention with these antiretroviral agents has led to a decline in the number of reported AIDS cases and has been shown to decrease morbidity and mortality associated with advanced AIDS. Prolonged treatment with these reverse transcriptase inhibitors eventually leads to the emergence of viral strains resistant to their antiviral effects. Recently, inhibitors of HIV-1 protease have emerged as a new class of HIV-1 chemotherapy. HIV-1 protease is an essential enzyme for viral infectivity and replication. Protease inhibitors have exhibited greater potency against HIV-1 in vitro than nucleoside analogs targeting HIV-1 RT. Inhibition of HIV-1 protease disrupts the creation of mature, infectious virus particles from chronically infected cells. This enzyme has become a viable target for therapeutic intervention and a candidate for combination therapy.

D. Transplant/Graft Rejection

The success of surgical transplantation of organs and tissue is largely dependent on the ability of the clinician to modulate the immune response of the transplant recipient. Specifically the immunological response directed against the transplanted foreign tissue must be controlled if the tissue is to survive and function. Currently, skin, kidney, liver, pancreas, lung and heart are the major organs or tissues with which allogeneic transplantations are performed. It has long been known that the normally functioning immune system of the transplant recipient recognizes the transplanted organ as “non-self” tissue and thereafter mounts an immune response to the presence of the transplanted organ. Left unchecked, the immune response will generate a plurality of cells and proteins that will ultimately result in the loss of biological functioning or the death of the transplanted organ.

This tissue/organ rejection can be categorized into three types: hyperacute, acute and chronic. Hyperacute rejection is essentially caused by circulating antibodies in the blood that are directed against the tissue of the transplanted organ (transplant). Hyperacute rejection can occur in a very short time—often in minutes—and leads to necrosis of the transplant. Acute graft rejection reaction is also immunologically mediated and somewhat delayed compared to hyperacute rejection. The chronic form of graft rejection that can occur years after the transplant is the result of a disease state commonly referred to as Graft Arterial Disease (GAD). GAD is largely a vascular disease characterized by neointimal proliferation of smooth muscle cells and mononuclear infiltrates in large and small vessels. This neointimal growth can lead to vessel fibrosis and occlusion, lessening blood flow to the graft tissue and resulting in organ failure. Current immunosuppressant therapies do not adequately prevent chronic rejection. Most of the gains in survival in the last decade are due to improvements in immunosuppressive drugs that prevent acute rejection. However, chronic rejection losses remain the same and drugs that can prevent it are a critical unmet medical need.

According to an embodiment of the invention, the methods described herein are useful in inhibiting innate immune response in cell graft or tissue graft rejection. Thus, the methods are useful for such grafted tissue as heart, lung, kidney, skin, cornea, liver, neuronal tissue or cell, or with stem cells, including hematopoetic or embryonic stem cells. In accordance herewith, treatment can be by inducing or increasing the expression or activity of DUX4-fl or its target genes such as DEFB103.

In accordance with another embodiment, the methods of this invention can be applied in conjunction with, or supplementary to, the customary treatments of transplant/graft rejection. Tissue graft and organ transplant recipients are customarily treated with one or more cytotoxic agents in an effort to suppress the transplant recipient's immune response against the transplanted organ or tissue. Current immunosuppressant drugs include: cyclosporin, tacrolimus (FK506), sirolimus (rapamycin), methotrexate, mycophenolic acid (mycophenolate mofetil), everolimus, azathiprine, steroids and NOX-100. All of these drugs have side effects (detailed below) that complicate their long-term use. For example, cyclosporin (cyclosporin A), a cyclic polypeptide consisting of 11 amino acid residues and produced by the fungus species Tolypocladium inflatum Gams, is currently the drug of choice for administration to the recipients of allogeneic kidney, liver, pancreas and heart (i.e., wherein donor and recipient are of the same species of mammals) transplants. However, administration of cyclosporin is not without drawbacks as the drug can cause kidney and liver toxicity as well as hypertension. Moreover, use of cyclosporin can lead to malignancies (such as lymphoma) as well as opportunistic infection due to the “global” nature of the immunosuppression it induces in patients receiving long term treatment with the drug, i.e., the hosts normal protective immune response to pathogenic microorganisms is down-regulated thereby increasing the risk of infections caused by these agents. FK506 (tacrolimus) has also been employed as an immunosuppressive agent as a stand-alone treatment or in combination. Although its immunosuppressive activity is 10 100 times greater than cyclosporin, it still has toxicity issues. Known side effects include kidney damage, seizures, tremors, high blood pressure, diabetes, high blood potassium, headache, insomnia, confusion, seizures, neuropathy, and gout. It has also been associated with miscarriages. Methotrexate is commonly added to the treatment of the cytotoxic agent. Methotrexate is given in small doses several times after the transplant. Although the combination of cyclosporin and methotrexate has been found to be effective in decreasing the severity of transplant rejection, there are side effects, such as mouth sores and liver damage. Severe transplant rejection can be treated with steroids. However, the side effects of steroids can be extreme, such as weight gain, fluid retention, elevated blood sugar, mood swings, and/or confused thinking.

E. Autoimmune Disease

“Autoimmune Disease” refers to those diseases which are commonly associated with the nonanaphylactic hypersensitivity reactions (Type II, Type III and/or Type IV hypersensitivity reactions) that generally result as a consequence of the subject's own humoral and/or cell-mediated immune response to one or more immunogenic substances of endogenous and/or exogenous origin. Such autoimmune diseases are distinguished from diseases associated with the anaphylactic (Type I or IgE-mediated) hypersensitivity reactions.

According to an embodiment of the invention, the methods described herein are useful in inhibiting the development of an autoimmune disease comprising inducing or increasing the expression or activity of DUX4-fl or its target genes such as DEFB103 in a subject.

Thus, the methods are useful for such autoimmune diseases as multiple sclerosis, systemic lupus erythematosus, type 1 diabetes, viral endocarditis, viral encephalitis, rheumatoid arthritis, Graves' disease, autoimmune thyroiditis, autoimmune myositis, and discoid lupus erythematosus.

The methods in accordance with this aspect of the invention may also comprise positive modulation of DUX4 or its target gene such as DEFB103A or DEFB103B. In certain aspect, if the subject has a disease such as an autoimmune disease that are caused by the misexpression of DUX4 (as determined by a higher level or activity than a normal control), such as FSHD, methods involving inhibiting expression or activity of DUX4 or its target genes (such as the DEFB103A or DEFB103 or cancer testis antigens) may applied to the subject for treatment.

### XII. KITS

Certain aspects of the present invention provide kits, such as diagnostic and therapeutic kits, as well as kits for preparing and/or screening antibodies. For example, a kit may comprise one or more pharmaceutical compositions as described herein and optionally instructions for their use. Kits may also comprise one or more devices for accomplishing administration of such compositions. For example, a subject kit may comprise a pharmaceutical composition and catheter for accomplishing direct intraarterial injection of the composition into a cancerous tumor. In other embodiments, a subject kit may comprise pre-filled ampoules of a protein isoform specific antibody construct, optionally formulated as a pharmaceutical, or lyophilized, for use with a delivery device.

Kits may comprise a container with a label. Suitable containers include, for example, bottles, vials, and test tubes. The containers may be formed from a variety of materials such as glass or plastic. The container may hold a composition which includes an antibody that is effective for therapeutic or non-therapeutic applications, such as described above. The label on the container may indicate that the composition is used for a specific therapy or non-therapeutic application, and may also indicate directions for either in vivo or in vitro use, such as those described above. The kit of the invention will typically comprise the container described above and one or more other containers comprising materials desirable from a commercial and user standpoint, including buffers, diluents, filters, needles, syringes, and package inserts with instructions for use.

### XIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

This Example demonstrates that DUX4-fl activates the expression of germline genes and binds uniformly throughout the genome.

### Background/Rationale:

Previously, the inventors identified two different DUX4 mRNA transcripts in human skeletal muscle, both at extremely low abundance: a full-length open reading frame mRNA (DUX4-fl) only detected in FSHD muscle and an internally spliced form of DUX4 mRNA (DUX4-s) that maintains the N-terminal double-homeobox domains but deletes the C-terminal domain and is detected in both control and FSHD muscle (Snider et al., 2010). Forced over-expression of DUX4-fl is toxic to cells, inducing apoptotic cell death (Kowaljow et al., 2007; Wallace et al., 2011), whereas forced over-expression of DUX4-s is not toxic to cultured human skeletal muscle cells (Geng et al., 2011).

To determine whether gene expression is regulated by DUX4-fl and/or DUX4-s in human muscle cells, as described in this Example, the inventors transduced primary myoblasts from a control individual (unaffected by muscle disease) with a lentiviral vector expressing either DUX4-fl or DUX4-s and performed expression microarrays.

### Methods and Materials:

Transduction of Primary Control Myoblasts with Lentiviral Vectors Expressing DUX4 fl or DUX4-s

Lentiviral vectors expressing either DUX4-fl or DUX4-s were constructed as follows:

The DUX4-fl and DUX4-s lentiviral constructs were generated by replacing the GFP gene in the lentiviral vector backbone “pRRLSIN.cPPT.PGK-GPF.WPRE”, as described in http://www.addgene.org/12252/” incorporated herein by reference, with the cDNA encoding DUX-4fl (SEQ ID NO:108 or SEQ ID NO:109), or with the cDNA encoding DUX4-s (SEQ ID NO:111).

Primary myoblasts from a control individual unaffected by muscle disease were transduced with the Lentiviral vectors expressing either DUX4-fl or DUX4-s. Primary human myoblasts were collected and cultured as previously described (Snider et al., 2010). Primary myoblasts were maintained at or below 70% confluency for proliferation. For differentiation, cells were allowed to reach 95-100% confluency in growth medium. Once confluency was reached, the cells were changed to differentiation medium (F10 media supplemented with 1% horse serum, 10 μg/mL insulin, and 10 μg/mL transferrin, penicillin/streptomycin) and maintained for 4 days. Human RD cells were grown in DMEM in 10% bovine calf serum (Hyclone) and penicillin/streptomycin. The primary myoblasts were transduced with lentivirus carrying DUX-fl, DUX4- or GFP (MOI=15).

### Expression Analysis

Expression microarrays were performed on the transduced cells at 24 hours after transduction as follows. Quadruplicate total RNA samples were collected from control human primary myoblasts transduced with lentivirus carrying DUX4-fl, DUX4-s or GFP (MOI=15) for 24 h. Samples were analyzed by Illumina Human Whole Genome microarrays. Probe intensities were corrected, normalized, and summarized by the Lumi package of Bioconductor (Du et al., 2008). Differentially expressed genes were identified by the LIMMA package of Bioconductor (Wettenhall and Smyth, 2004). Gene set enrichment analysis (GSEA) was performed using the Bioconductor GOstats package (Falcon and Gentleman, 2007).

### Microarray Gene Target Validation by RT-PCR

RNA was collected from cultured control skeletal muscle either transduced with a DUX4-fl expressing lentivirus (+) or not transduced (−). RPL13A was used as an internal standard. Total RNA was treated with DNase using TURBO DNA-free kit (Ambion) according to manufacturer's protocol. One μg of DNase-treated RNA was reverse transcribed to first strand cDNA with SuperScript III and anchored oligo dT (Invitrogen) at 52° C. for 1 h. Residual RNA was digested with RNase H at 37° C. for 20 min. cDNA was used in various PCR and real-time PCR reactions with primers listed below.

cDNA from DUX4-fl transduced or untransduced primary myoblasts was diluted 1:5 and used in PCR reactions with Platinum Taq polymerase (Invitrogen) with conditions of 55° C. annealing temperature and 35 cycles. The primers shown below were designed to span exon-exon junctions where possible. Primers from select genes were also used in real-time PCR reactions to examine endogenous expression of targets in FSHD versus control samples described separately.

| Gene | Forward primer | Reverse primer |
|---|---|---|
| name | sequence | sequence |
| TRIM43 | ACCCATCACTGGACTGGTGT | CACATCCTCAAAGAGCCTGA |
| | (SEQ ID NO: 113) | (SEQ ID NO: 114) |
| PRAMEF1 | GCTGGAACACCTTCAGTTGC | AGTTCTCCAAGGGGTTCTGG |
| | (SEQ ID NO: 115) | (SEQ ID NO: 116) |
| RFPL4B | GAGACGTAGGCTTCGGATCTT | GGCTGAATTCAAGTGGGTCT |
| | (SEQ ID NO: 117) | (SEQ ID NO: 118) |
| ZSCAN4 | TGGAAATCAAGTGGCAAAAA | CTGCATGTGGACGTGGAC |
| | (SEQ ID NO: 119) | (SEQ ID NO: 120) |
| KHDC1 | ACCAATGGTGTTTCACATGG | TGAATAAGGGTGTGGCTGTG |
| | (SEQ ID NO: 121) | (SEQ ID NO: 122) |
| RFPL2 | CCCACATCAAGGAACTGGAG | TGTTGGCATCCAAGGTCATA |
| | (SEQ ID NO: 123) | (SEQ ID NO: 124) |
| CXCR4 | CGTGGAACGTTTTTCCTGTT | GGTGCTGAAATCAACCCACT |
| | (SEQ ID NO: 125) | (SEQ ID NO: 126) |
| WDR33 | GGTCCCACCTATAGGAATGT | GACCAAGCGTCTTCCTTCTG |
| | TG | (SEQ ID NO: 128) |
| | (SEQ ID NO: 127) | |
| MBD3L2 | GCGTTCACCTCTTTTCCAAG | GCCATGTGGATTTCTCGTTT |
| | (SEQ ID NO: 129) | (SEQ ID NO: 130) |
| CCNA1 | TGAAGCAGATCCATTCTTGA | ACCCTGTAAATGCAGCAAGG |
| | AA | (SEQ ID NO: 132) |
| | (SEQ ID NO: 131) | |
| TRIM48 | TGAATGTGGAAACCACCAGA | GTTGAGCCTGTCCCTCAGTC |
| | (SEQ ID NO: 133) | (SEQ ID NO: 134) |
| PRAMEF2 | ACCTTCTTCAGTGGGCACCT | TGGGAACTGGGAGAGACACT |
| | (SEQ ID NO: 135) | (SEQ ID NO: 136) |
| IFI27 | CCATAGCAGCCAAGATGATG | GAACTTGGTCAATCCGGAGA |
| | (SEQ ID NO: 137) | (SEQ ID NO: 138) |
| TESK2 | GCAGGAGAGGGATAGGAAGC | CTTGTGGGGGATCTTGTCAT |
| | (SEQ ID NO: 139) | (SEQ ID NO: 140) |
| PELI1 | CTAAGGCAAATGGGGTGAAG | TCTGGGCCCGAGATAAAGTA |
| | (SEQ ID NO: 141) | (SEQ ID NO: 142) |
| FRG2B | GTCCAGCTCATATCGGGAAA | GCTGCACTCCTTTTCTGGAC |
| | (SEQ ID NO: 143) | (SEQ ID NO: 144) |
| HSPA2 | CTTCTGCCGTGATTGTGAGG | CCAGGGGGTCTAGGTAGGAG |
| | (SEQ ID NO: 145) | (SEQ ID NO: 146) |
| RPL13A | AACCTCCTCCTTTTCCAAGC | GCAGTACCTGTTTAGCCACGA |
| | (SEQ ID NO: 147) | (SEQ ID NO: 148) |

### Results:

### Identification of Genes Regulated by DUX4 in Human Primary Myoblasts

At 24 hours after transduction, DUX4-fl increased the expression of 1071 genes and decreased the expression of 837 genes compared to a control myoblast population similarly infected with a GFP expressing lentivirus (2-fold change and FDR<0.01); whereas DUX4-s increased the expression of 159 genes and decreased expression of 45 genes. The full set of genes regulated by DUX4-fl or DUX4-s is in Table 1, provided in the appendix.

Table 1 (included as an Appendix) shows the expression array analysis of DUX4-fl and DUX4-s in cultured human skeletal muscle.

Using a more stringent 3-fold criteria (>1.584 log₂-fold change and FDR<0.01), 466 genes were increased and 244 decreased by DUX4-fl; and 37 were increased and one decreased by DUX4-s. Only two annotated genes were increased 3-fold or more by both (CCNA1, MAP2), and none were decreased 3-fold or more by both.

In view of the fact that Table 1 lists the fold-change in log 2, a value of “3” in Table 1 would be an 8-fold change. As shown in Table 1, 164 genes were identified that increase 8-fold or more, 107 increased 16-fold or more, and 72 genes increased 32-fold or more (i.e. log 2fc≧5).

The 107 genes that were found to be increased by at least 16-fold or greater in the presence of DUX4-fl are useful as FSHD biomarkers and are provided below in Table 2:

TABLE 2
FSHD Biomarker Genes upregulated by DUX4-fl

| SEQ | | | | |
|---|---|---|---|---|
| ID | | | | |
| NO: | Symbol | RefSeq* | full_fc | full_pval |
| 1 | RFPL1S | NR_002727.1 | 8.395820858 | 4.68E−27 |
| 2 | LOC643263 | XR_016355.1 | 8.345299826 | 5.16E−27 |
| 3 | RFPL4B | NM_001013734.2 | 8.340345819 | 5.13E−28 |
| 4 | LOC390031 | XM_372343.1 | 8.330613566 | 5.02E−28 |
| 5 | ZSCAN4 | NM_152677.1 | 8.321990102 | 1.94E−28 |
| 6 | LOC340970 | XR_038494.1 | 8.315993278 | 3.20E−28 |
| 7 | LOC136157 | XM_069743.3 | 8.298510216 | 1.98E−27 |
| 8 | LOC643445 | XR_038080.1 | 8.249957558 | 1.44E−28 |
| 9 | LOC729458 | XM_001130308.2 | 8.246687197 | 2.30E−27 |
| 10 | LOC653192 | XM_926437.2 | 8.228018909 | 2.48E−27 |
| 11 | LOC645669 | XM_928680.1 | 8.202022481 | 1.85E−27 |
| 12 | LOC391769 | XM_001713901.1 | 8.189552468 | 3.39E−27 |
| 13 | LOC196120 | XM_114987.3 | 8.178925427 | 2.42E−27 |
| 14 | LOC651308 | XM_940443.1 | 8.168661444 | 4.84E−25 |
| 15 | RFPL3 | NM_001098535.1 | 8.144474769 | 9.29E−29 |
| 16 | PRAMEF1 | NM_023013.1 | 8.072400408 | 3.19E−27 |
| 17 | LOC100134199 | XM_001719549.1 | 8.048036849 | 6.76E−28 |
| 18 | SPRYD5 | NM_032681.1 | 8.044967325 | 5.44E−28 |
| 19 | LOC284428 | XM_208203.5 | 8.022522551 | 1.38E−26 |
| 20 | LOC642362 | XM_925891.1 | 8.015825025 | 1.66E−27 |
| 21 | KHDC1L | NM_001126063.2 | 8.012411091 | 1.06E−27 |
| 22 | LOC653656 | XM_928688.3 | 7.897231482 | 5.40E−28 |
| 23 | TRIM48 | NM_024114.2 | 7.880137061 | 5.54E−26 |
| 24 | LOC653657 | XM_928697.2 | 7.856575803 | 3.03E−27 |
| 25 | PRAMEF12 | NM_001080830.1 | 7.801903788 | 1.84E−25 |
| 26 | LOC441584 | XM_497258.1 | 7.781378819 | 4.75E−27 |
| 27 | LOC730974 | XR_037751.1 | 7.715075519 | 9.06E−26 |
| 28 | PRAMEF7 | NM_001012277.1 | 7.631155888 | 1.22E−27 |
| 29 | MBD3L2 | NM_144614.2 | 7.622770725 | 3.46E−26 |
| 30 | LOC440040 | XM_495873.4 | 7.533852122 | 2.79E−27 |
| 31 | CCNA1 | NM_003914.2 | 7.525825564 | 1.10E−26 |
| 32 | PRAMEF13 | XM_001713933.1 | 7.421574077 | 3.37E−27 |
| 33 | LOC342900 | XM_001129035.1 | 7.391093477 | 4.53E−28 |
| 34 | LOC340096 | XM_293943.2 | 7.38245832 | 9.80E−25 |
| 35 | PRAMEF5 | NM_001013407.1 | 7.34950535 | 3.80E−23 |
| 36 | RFPL2 | NM_006605.1 | 7.293384138 | 3.38E−25 |
| 37 | PRAMEF9 | NM_001010890.1 | 7.130773908 | 7.31E−25 |
| 38 | LOC100134006 | XM_001725030.1 | 7.08721139 | 7.77E−27 |
| 39 | PRAMEF4 | NM_001009611.1 | 7.060257208 | 2.65E−24 |
| 40 | PRAMEF15 | XM_001713659.1 | 7.000221925 | 4.98E−26 |
| 41 | LOC100131392 | XM_001713681.1 | 6.975776511 | 9.12E−25 |
| 42 | NP | NM_000270.1 | 6.960976026 | 4.12E−27 |
| 43 | LOC399939 | XM_374919.3 | 6.930795087 | 9.92E−27 |
| 44 | LOC642148 | XR_019607.1 | 6.85089804 | 8.92E−25 |
| 45 | LOC729384 | NM_001105522.1 | 6.831960625 | 2.20E−27 |
| 46 | ZNF705A | NM_001004328.1 | 6.831813353 | 3.44E−27 |
| 47 | C6orf148 | NM_030568.2 | 6.759160491 | 7.93E−25 |
| 48 | TRIM49 | NM_020358.2 | 6.551062725 | 3.44E−26 |
| 49 | DEFB103A | NM_001081551.2 | 6.441860402 | 1.15E−25 |
| 50 | PRAMEF2 | NM_023014.1 | 6.439143984 | 2.12E−25 |
| 51 | RFPL1 | NM_021026.2 | 6.264001827 | 8.17E−25 |
| 52 | LOC100133984 | XM_001723079.1 | 6.203778673 | 8.08E−25 |
| 53 | LOC642127 | XM_936272.2 | 6.112037689 | 6.46E−24 |
| 54 | CA2 | NM_000067.1 | 6.091135387 | 5.91E−24 |
| 55 | PRAMEF10 | NM_001039361.1 | 6.063554254 | 1.77E−23 |
| 56 | LOC646698 | XM_929644.2 | 6.012022368 | 9.84E−24 |
| 57 | LOC729516 | XR_038445.1 | 5.954919316 | 1.03E−25 |
| 58 | PRAMEF11 | XM_001714028.1 | 5.93984508 | 1.97E−24 |
| 59 | CSAG3 | NM_001129826.1 | 5.871224381 | 6.50E−24 |
| 60 | PRAMEF6 | NM_001010889.2 | 5.82553958 | 8.31E−25 |
| 61 | LOC391764 | XM_373076.3 | 5.820931052 | 1.05E−24 |
| 62 | TRIM43 | NM_138800.1 | 5.805862854 | 1.43E−20 |
| 63 | LOC391742 | XM_373056.1 | 5.733140049 | 1.50E−25 |
| 64 | LOC391766 | XM_373077.2 | 5.723821554 | 3.38E−25 |
| 65 | ZNF296 | NM_145288.1 | 5.536035027 | 9.82E−25 |
| 66 | SLC34A2 | NM_006424.2 | 5.513611409 | 5.77E−22 |
| 67 | LOC391767 | XM_373078.1 | 5.491772222 | 3.46E−21 |
| 68 | LOC729368 | XM_001130065.2 | 5.416246795 | 1.19E−23 |
| 69 | LOC440563 | NM_001136561.1 | 5.312436177 | 3.77E−22 |
| 70 | LOC646754 | XM_929704.2 | 5.110280465 | 3.49E−22 |
| 71 | LOC654101 | XM_939354.1 | 5.033863949 | 5.71E−21 |
| 72 | LOC729731 | XM_001131140.1 | 5.007248294 | 1.46E−23 |
| 73 | HIST2H3A | NM_001005464.2 | 4.94502277 | 2.03E−21 |
| 74 | TRIM64 | XM_061890.11 | 4.943161345 | 2.26E−23 |
| 75 | LOC402207 | XM_377884.2 | 4.902732221 | 6.85E−23 |
| 76 | LOC729700 | XM_001131081.1 | 4.817203 | 1.04E−23 |
| 77 | LOC645558 | XM_928577.2 | 4.802893 | 1.18E−22 |
| 78 | LOC642219 | XM_936370.2 | 4.798732 | 2.95E−20 |
| 79 | PRAMEF20 | NM_001099852.1 | 4.795166 | 1.03E−23 |
| 80 | HBA1 | NM_000558.3 | 4.786546 | 5.55E−23 |
| 81 | TRIM53 | XR_041244.1 | 4.777538 | 1.16E−22 |
| 82 | LOC399940 | NM_001136118.1 | 4.726731 | 6.54E−22 |
| 83 | HBA2 | NM_000517.3 | 4.72082 | 4.76E−24 |
| 84 | LOC646103 | XM_377879.3 | 4.658033 | 6.41E−21 |
| 85 | LOC732393 | XR_015873.1 | 4.637178 | 1.36E−21 |
| 86 | LOC100133446 | XM_001717965.1 | 4.634629 | 4.84E−23 |
| 87 | LOC100131539 | XM_001724873.1 | 4.629059 | 6.67E−21 |
| 88 | C12orf50 | NM_152589.1 | 4.521768 | 6.50E−23 |
| 89 | OR2T34 | NM_001001821.1 | 4.519029 | 5.05E−23 |
| 90 | TPRX1 | NM_198479.2 | 4.48321 | 1.10E−23 |
| 91 | LOC402199 | XM_377875.2 | 4.39249 | 3.01E−21 |
| 92 | LOC646066 | XM_116384.2 | 4.391241 | 2.75E−21 |
| 93 | ART3 | NM_001179.3 | 4.363323 | 2.34E−22 |
| 94 | RFPL4A | XM_001719234.1 | 4.347532 | 6.99E−22 |
| 95 | LOC401860 | XM_377445.3 | 4.272237 | 3.19E−21 |
| 96 | NXF1 | NM_006362.4 | 4.233044 | 3.92E−22 |
| 97 | LOC729706 | XM_001131091.1 | 4.227191 | 1.26E−21 |
| 98 | PRAMEF17 | XM_938420.2 | 4.223086 | 5.13E−20 |
| 99 | SFRS2B | NM_032102.2 | 4.215303 | 3.27E−22 |
| 100 | RN5S9 | NR_023371.1 | 4.191231 | 9.29E−23 |
| 101 | PPP2R2B | NM_181677.1 | 4.130028 | 1.09E−21 |
| 102 | ZNF217 | NM_006526.2 | 4.113561 | 6.85E−22 |
| 103 | ENTPD8 | NM_001033113.1 | 4.072927 | 1.36E−21 |
| 104 | LOC647827 | XR_018213.1 | 4.053399 | 4.92E−20 |
| 105 | THOC4 | XM_001134346.1 | 4.034801 | 7.79E−22 |
| 106 | LOC729694 | XM_001131061.1 | 4.028728 | 2.38E−19 |
| 107 | DEFB103BA | NM_018661.3 | | |

*Genbank reference No. as accessed on Jul. 22, 2011.

A representative sample of genes activated by DUX4-fl is shown in Table 3.

TABLE 3
Representative genes induced by DUX4-fl

| | | | |
|---|---|---|---|
| | Log₂ | Log₂ | |
| | DUX4-fl | DUX4-s | |
| Category | Fc* | Fc* | Comments |

Germline and Stem Cells

| | | | |
|---|---|---|---|
| ZSCAN4 | 8.3 | 0.0 | Genome stability, telomere length |
| (SEQ ID NO: 5) | | | |
| PRAMEF1 | 8.1 | 0.1 | Melanoma antigen family |
| (SEQ ID NO: 16) | | | |
| SPRYD5 | 8.0 | −0.1 | Expressed in oocyte |
| (SEQ ID NO: 18) | | | |
| KHDC1L (SEQ | 8.0 | −0.1 | KH RNA binding domain |
| ID NO: 21) | | | |
| MBD3L2 (SEQ | 7.6 | 0.0 | Methyl-CpG-binding protein |
| ID NO: 29) | | | |
| ZNF705A | 6.8 | −0.1 | Zinc finger protein |
| (SEQ ID NO: 46) | | | |
| TRIM43 | 5.8 | 0.0 | Preimplantation embryo |
| (SEQ ID NO: 62) | | | |
| TPRX1 | 4.5 | −0.1 | Homeobox protein |
| (SEQ ID NO: 90) | | | |
| ZNF217 | 4.1 | −0.3 | Expressed in cancer stem cells |
| (SEQ ID No: 102) | | | |
| HSPA2 | 3.7 | −0.3 | Chaperone, heat shock 70 kd |
| JUP | 3.2 | −0.1 | expressed in germline and testicular cancers |
| FGFR3 | 3.1 | 0.0 | Expressed in spermatogonia |
| CD24 | 2.6 | −0.4 | Stem cell marker |
| SLC2A14 | 2.4 | 0.2 | Spermatogenesis |
| ID2 | 2.3 | 0.3 | Negative regulator of cell differentiation |
| PVRL3 | 2.2 | 0.4 | Spermatid-sertoli junction |
| HOXB2 | 2.2 | 0.0 | Anterior-posterior axis development |
| ZSCAN2 | 2.2 | −0.2 | Spermatogenesis and embryonic |
| | | | development |

RNA Processing

| | | | |
|---|---|---|---|
| SFRS2B (SEQ | 4.2 | −0.3 | Splicing |
| ID NO: 99) | | | |
| THOC4 | 4.0 | −0.2 | Splicing, RNA transport |
| (SEQ ID NO: 105) | | | |
| ZNHIT6 | 3.5 | 0.3 | sno-RNA processing |
| DBR1 | 3.4 | 0.2 | RNA lariat debranching enzyme |
| TFIP11 | 3.2 | 0.1 | Splicesome assembly |
| CWC15 | 2.6 | 0.1 | Spliceosome-associated |
| ARS2 | 2.6 | −0.2 | miRNA processing |
| PABPN1 | 2.6 | −0.3 | PolyA binding |
| SFRS17A | 2.5 | 0.2 | Spliceosome-associated |
| RMRP | 2.3 | 0.1 | Mitochondrial RNA processing |
| SNIP1 | 2.1 | −0.2 | miRNA biogenesis |
| RPPH1 | 2.0 | 0.2 | tRNA processing |
| RNGTT | 2.0 | −0.6 | mRNA processing |

Ubiquitin Pathway

| | | | |
|---|---|---|---|
| SIAH1 | 3.7 | −0.1 | Targets TRF2 telomere maintenance |
| FBXO33 | 3.2 | 0.2 | E3 ubiquitin-protein ligase complex |
| PELI1 | 2.9 | 0.1 | E3 ligases involved in innate immunity |
| USP29 | 2.6 | −0.1 | Ubiquitin-specific peptidase |
| ARIH1 | 2.2 | 0.8 | Ubiquitin-conjugating enzyme E2 binding |
| | | | protein |
| TRIM23 | 2.2 | 0.6 | E3 ubiquitin ligase involved in immunity |

Immunity and Innate Defense

| | | | |
|---|---|---|---|
| DEFB103B | 6.4 | 0.1 | Innate defense |
| (SEQ ID NO: 49) | | | |
| IFRD1 | 3.0 | −0.2 | Interferon-related developmental regulator |
| CXADR | 2.5 | −0.1 | Leukocyte migration |
| CBARA1 | 2.1 | −0.2 | T-helper 1-mediated autoreactivity |
| SON | 2.1 | −0.3 | Viral response |
| CXCR4 | 2.0 | −0.1 | Chemotaxis |

General Transcription

| | | | |
|---|---|---|---|
| GTF2F1 | 3.2 | 0.3 | General transcription factor IIF |
| MED26 | 2.1 | 0.1 | RNA Pol II mediator complex |
| RRN3 | 2.1 | 0.1 | RNA Pol I preinitiation complex |

Cancer Expressed

| | | | |
|---|---|---|---|
| CSAG3 | 5.9 | 0.1 | Chondrosarcoma-associated gene |
| (SEQ ID NO: 59) | | | |
| SLC34A2 | 5.5 | 0.0 | Breast cancer biomarker |
| (SEQ ID NO: 66) | | | |
| PNMA6B | 3.6 | −0.2 | Paraneoplastic antigen |
| CSE1L | 2.9 | 0.1 | Cellular apoptosis susceptibility protein |
| AMACR | 2.7 | 0.1 | Prostate cancer biomarker |

Other

| | | | |
|---|---|---|---|
| FLJ45337 | 3.7 | −0.2 | Endogenous retrovirus |
| HNRNPCL1 | 3.5 | −0.1 | Nucleosome assembly |
| SPTY2D1 | 3.3 | −0.3 | Suppressor of ty retrotransposons in yeast |
| MGC10997 | 2.4 | −0.3 | Endogenous retrotransposon |

The Gene Ontology (GO) terms significantly enriched in 3-fold up-regulated genes by DUX4-fl included categories such as RNA polymerase II mediator complexes, RNA splicing and processing, and gamete/spermatogenesis, as shown in Table 4.

Table 4 shows the gene Ontology analysis of genes up-regulated by DUX4-fl.

TABLE 4
Gene Ontology Analysis of genes upregulated by DUX4-fl

| GOID | Pvalue | OddsRatio | ExpCount | Count | Size | Term | Ontology | Geneset |
|---|---|---|---|---|---|---|---|---|
| GO: 0016455 | 0.000191764 | 11.43687515 | 0.561257787 | 5 | 22 | RNA polymerase II transcription | MF | fc3.up |
| | | | | | | mediator activity | | |
| GO: 0016592 | 0.000328786 | 9.937015504 | 0.623932435 | 5 | 25 | mediator complex | CC | fc3.up |
| GO: 0016607 | 3.11E−07 | 6.292245907 | 2.620516227 | 14 | 105 | nuclear speck | CC | fc3.up |
| GO: 0000398 | 0.000279448 | 6.248262359 | 1.294881345 | 7 | 52 | nuclear mRNA splicing, via spliceosome | BP | fc3.up |
| GO: 0007411 | 0.004634577 | 5.074925075 | 1.10245249 | 5 | 44 | axon guidance | BP | fc3.up |
| GO: 0003729 | 0.005361849 | 4.884960159 | 1.140029688 | 5 | 45 | mRNA binding | MF | fc3.up |
| GO: 0006986 | 0.002679577 | 4.752207792 | 1.406677266 | 6 | 56 | response to unfolded protein | BP | fc3.up |
| GO: 0005681 | 2.10E−05 | 4.514666667 | 3.244448662 | 13 | 130 | spliceosomal complex | CC | fc3.up |
| GO: 0000375 | 0.000181802 | 4.508736326 | 2.486804452 | 10 | 99 | RNA splicing, via transesterification | BP | fc3.up |
| | | | | | | reactions | | |
| GO: 0048762 | 0.007434327 | 4.483738245 | 1.230842607 | 5 | 49 | mesenchymal cell differentiation | BP | fc3.up |
| GO: 0050657 | 0.000411547 | 4.442982456 | 2.26073132 | 9 | 90 | nucleic acid transport | BP | fc3.up |
| GO: 0051236 | 0.000411547 | 4.442982456 | 2.26073132 | 9 | 90 | establishment of RNA localization | BP | fc3.up |
| GO: 0008380 | 4.17E−05 | 3.948955478 | 3.96665952 | 14 | 163 | RNA splicing | BP | fc3.up |
| GO: 0019222 | 0.022673693 | 3.317567568 | 1.622481442 | 5 | 70 | regulation of metabolic process | BP | fc3.up |
| GO: 0010720 | 0.023351201 | 3.282327586 | 1.632750397 | 5 | 65 | positive regulation of cell development | BP | fc3.up |
| GO: 0006397 | 0.001604973 | 2.933267749 | 4.427103404 | 12 | 183 | mRNA processing | BP | fc3.up |
| GO: 0010769 | 0.038069546 | 2.851386807 | 1.858823529 | 5 | 74 | regulation of cell morphogenesis involved | BP | fc3.up |
| | | | | | | in differentiation | | |
| GO: 0051169 | 0.00252323 | 2.765977011 | 4.67217806 | 12 | 186 | nuclear transport | BP | fc3.up |
| GO: 0001655 | 0.043864833 | 2.734187109 | 1.93257471 | 5 | 77 | urogenital system development | BP | fc3.up |
| GO: 0007018 | 0.043997794 | 2.731681034 | 1.93418124 | 5 | 77 | microtubule-based movement | BP | fc3.up |
| GO: 0002521 | 0.027827111 | 2.514466403 | 2.938950715 | 7 | 117 | leukocyte differentiation | BP | fc3.up |
| GO: 0007275 | 0.021259682 | 2.127133015 | 5.476301779 | 11 | 233 | multicellular organismal development | BP | fc3.up |
| GO: 0044419 | 0.013115877 | 2.123638693 | 6.473314905 | 13 | 258 | interspecies interaction between | BP | fc3.up |
| | | | | | | organisms | | |
| GO: 0007276 | 0.046093024 | 2.102561097 | 3.977549711 | 8 | 159 | gamete generation | BP | fc3.up |
| GO: 0005654 | 0.011464976 | 2.095199054 | 7.064796326 | 14 | 309 | nucleoplasm | CC | fc3.up |
| GO: 0007283 | 0.042362233 | 2.035263158 | 4.621939587 | 9 | 184 | spermatogenesis | BP | fc3.up |
| GO: 0006915 | 0.038108478 | 1.992664746 | 5.256515307 | 10 | 209 | apoptosis | BP | fc3.up |
| GO: 0070013 | 3.55E−05 | 1.856371356 | 40.15629152 | 65 | 1609 | intracellular organelle lumen | CC | fc3.up |
| GO: 0032504 | 0.028221469 | 1.849315475 | 7.937678855 | 14 | 316 | multicellular organism reproduction | BP | fc3.up |
| GO: 0031974 | 8.33E−05 | 1.794502609 | 41.2793699 | 65 | 1654 | membrane-enclosed lumen | CC | fc3.up |
| GO: 2000026 | 0.045419104 | 1.721140016 | 8.484942332 | 14 | 341 | regulation of multicellular organismal | BP | fc3.up |
| | | | | | | development | | |
| GO: 0005634 | 2.40E−05 | 1.680057275 | 99.52516619 | 132 | 4000 | nucleus | CC | fc3.up |
| GO: 0003723 | 0.026131966 | 1.675158085 | 12.53272055 | 20 | 512 | RNA binding | MF | fc3.up |
| GO: 0050794 | 0.003573262 | 1.663024053 | 31.57386454 | 46 | 1223 | regulation of cellular process | BP | fc3.up |
| GO: 0005730 | 0.018174998 | 1.641408991 | 16.04754223 | 25 | 643 | nucleolus | CC | fc3.up |
| GO: 0060255 | 0.00375921 | 1.575747148 | 38.14639346 | 54 | 1498 | regulation of macromolecule metabolic | BP | fc3.up |
| | | | | | | process | | |
| GO: 0034641 | 0.001043025 | 1.543947976 | 72.70723982 | 95 | 2819 | cellular nitrogen compound metabolic | BP | fc3.up |
| | | | | | | process | | |

GOID: Gene Ontology ID
ExpCount: expected count
Count: actual count in data set
Size: size of GO term
Term: GO term
Geneset: genes upregulated by DUX4-fl by 3x fold change or more

The down-regulated genes represent the immune response pathways, as shown in TABLE 5.

Table 5 shows the Gene Ontology analysis of genes down-regulated by DUX4-fl.

TABLE 5
Gene Ontology Analysis of genes downregulated by DUX4-fl

| GOID | Pvalue | OddsRatio | ExpCount | Count | Size | Term | Ontology | Geneset |
|---|---|---|---|---|---|---|---|---|
| GO: 0008009 | 4.46E−06 | 17.99454 | 0.461189 | 6 | 22 | chemokine activity | MF | fc3.down |
| GO: 0002253 | 3.77E−05 | 17.02899 | 0.40639 | 5 | 18 | activation of immune response | BP | fc3.down |
| GO: 0009615 | 9.02E−17 | 15.93194 | 1.893662 | 21 | 84 | response to virus | BP | fc3.down |
| GO: 0002541 | 8.86E−05 | 13.69643 | 0.478537 | 5 | 21 | activation of plasma proteins involved in | BP | fc3.down |
| | | | | | | acute inflammatory response | | |
| GO: 0050792 | 0.000113 | 12.88936 | 0.501325 | 5 | 22 | regulation of viral reproduction | BP | fc3.down |
| GO: 0006955 | 2.35E−17 | 11.47143 | 3.073569 | 26 | 150 | immune response | BP | fc3.down |
| GO: 0045087 | 6.12E−09 | 10.07058 | 1.626719 | 13 | 73 | innate immune response | BP | fc3.down |
| GO: 0006952 | 0.000475 | 8.863863 | 0.664982 | 5 | 34 | defense response | BP | fc3.down |
| GO: 0050900 | 0.000804 | 7.869162 | 0.74721 | 5 | 33 | leukocyte migration | BP | fc3.down |
| GO: 0019882 | 0.000879 | 7.688316 | 0.761086 | 5 | 34 | antigen processing and presentation | BP | fc3.down |
| GO: 0048519 | 9.32E−06 | 7.615385 | 1.400399 | 9 | 68 | negative regulation of biological process | BP | fc3.down |
| GO: 0006959 | 0.000294 | 7.570055 | 0.93004 | 6 | 41 | humoral immune response | BP | fc3.down |
| GO: 0051384 | 0.000708 | 6.296703 | 1.089983 | 6 | 48 | response to glucocorticoid stimulus | BP | fc3.down |
| GO: 0042542 | 0.00279 | 5.753133 | 0.979862 | 5 | 43 | response to hydrogen peroxide | BP | fc3.down |
| GO: 0048545 | 0.004675 | 5.03714 | 1.101595 | 5 | 49 | response to steroid hormone stimulus | BP | fc3.down |
| GO: 0001664 | 0.000882 | 5.011867 | 1.551271 | 7 | 74 | G-protein-coupled receptor binding | MF | fc3.down |
| GO: 0060326 | 0.004951 | 4.965368 | 1.116587 | 5 | 49 | cell chemotaxis | BP | fc3.down |
| GO: 0006916 | 1.41E−05 | 4.709181 | 3.12766 | 13 | 140 | anti-apoptosis | BP | fc3.down |
| GO: 0023038 | 0.003257 | 4.534895 | 1.4584 | 6 | 64 | signal initiation by diffusible mediator | BP | fc3.down |
| GO: 0044419 | 7.31E−09 | 4.498795 | 6.540011 | 25 | 287 | interspecies interaction between organisms | BP | fc3.down |
| GO: 0050776 | 0.000861 | 4.411115 | 2.005544 | 8 | 90 | regulation of immune response | BP | fc3.down |
| GO: 0016757 | 0.008193 | 4.336898 | 1.254502 | 5 | 60 | transferase activity, transferring | MF | fc3.down |
| | | | | | | glycosyl groups | | |
| GO: 0051604 | 0.001941 | 4.336606 | 1.777424 | 7 | 78 | protein maturation | BP | fc3.down |
| GO: 0032496 | 0.008947 | 4.250743 | 1.283484 | 5 | 57 | response to lipopolysaccharide | BP | fc3.down |
| GO: 0002684 | 7.38E−05 | 4.243549 | 3.160098 | 12 | 139 | positive regulation of immune system | BP | fc3.down |
| | | | | | | process | | |
| GO: 0006935 | 0.010797 | 4.042572 | 1.34314 | 5 | 60 | chemotaxis | BP | fc3.down |
| GO: 0001871 | 0.004449 | 3.681051 | 2.054385 | 7 | 98 | pattern binding | MF | fc3.down |
| GO: 0006954 | 0.00274 | 3.618056 | 2.400128 | 8 | 111 | inflammatory response | BP | fc3.down |
| GO: 0005126 | 0.005119 | 3.579944 | 2.107496 | 7 | 101 | cytokine receptor binding | MF | fc3.down |
| GO: 0005615 | 4.47E−07 | 3.542467 | 8.041374 | 25 | 380 | extracellular space | CC | fc3.down |
| GO: 0009617 | 0.010017 | 3.519614 | 1.836402 | 6 | 81 | response to bacterium | BP | fc3.down |
| GO: 0007584 | 0.005785 | 3.50269 | 2.158862 | 7 | 95 | response to nutrient | BP | fc3.down |
| GO: 0030246 | 0.00614 | 3.452381 | 2.179423 | 7 | 105 | carbohydrate binding | MF | fc3.down |
| GO: 0009605 | 7.19E−05 | 3.377922 | 5.255012 | 16 | 239 | response to external stimulus | BP | fc3.down |
| GO: 0007568 | 0.007331 | 3.339047 | 2.255962 | 7 | 99 | aging | BP | fc3.down |
| GO: 0048584 | 0.004435 | 3.323799 | 2.596461 | 8 | 116 | positive regulation of response to stimulus | BP | fc3.down |
| GO: 0007626 | 0.001116 | 3.260458 | 3.668786 | 11 | 161 | locomotory behavior | BP | fc3.down |
| GO: 0051100 | 0.025205 | 3.204482 | 1.663487 | 5 | 73 | negative regulation of binding | BP | fc3.down |
| GO: 0008285 | 0.000348 | 3.167963 | 4.840465 | 14 | 216 | negative regulation of cell proliferation | BP | fc3.down |
| GO: 0031668 | 0.026548 | 3.157695 | 1.686275 | 5 | 74 | cellular response to extracellular stimulus | BP | fc3.down |
| GO: 0060548 | 5.55E−05 | 3.072478 | 6.859036 | 19 | 301 | negative regulation of cell death | BP | fc3.down |
| GO: 0007267 | 0.002407 | 2.9375 | 4.040065 | 11 | 179 | cell-cell signaling | BP | fc3.down |
| GO: 0007155 | 0.002567 | 2.911979 | 4.074034 | 11 | 182 | cell adhesion | BP | fc3.down |
| GO: 0008083 | 0.035758 | 2.892365 | 1.823791 | 5 | 87 | growth factor activity | MF | fc3.down |
| GO: 0002521 | 0.017372 | 2.78715 | 2.666137 | 7 | 117 | leukocyte differentiation | BP | fc3.down |
| GO: 0005625 | 0.002584 | 2.749994 | 4.676694 | 12 | 221 | soluble fraction | CC | fc3.down |
| GO: 0005576 | 1.10E−07 | 2.677219 | 19.78601 | 45 | 935 | extracellular region | CC | fc3.down |
| GO: 0002252 | 0.048324 | 2.650262 | 1.983392 | 5 | 90 | immune effector process | BP | fc3.down |
| GO: 0005792 | 0.017776 | 2.556128 | 3.301196 | 8 | 156 | microsome | CC | fc3.down |
| GO: 0090046 | 0.047437 | 2.399631 | 2.620562 | 6 | 115 | regulation of transcription regulator activity | BP | fc3.down |
| GO: 0060537 | 0.034573 | 2.390475 | 3.076312 | 7 | 135 | muscle tissue development | BP | fc3.down |
| GO: 0003714 | 0.049119 | 2.374029 | 2.641353 | 6 | 126 | transcription corepressor activity | MF | fc3.down |
| GO: 0040011 | 0.010723 | 2.363266 | 4.94011 | 11 | 218 | locomotion | BP | fc3.down |
| GO: 0080134 | 0.015205 | 2.237124 | 5.195548 | 11 | 228 | regulation of response to stress | BP | fc3.down |
| GO: 0010942 | 0.004177 | 2.182383 | 8.340223 | 17 | 366 | positive regulation of cell death | BP | fc3.down |
| GO: 0050896 | 0.00296 | 2.147643 | 10.29764 | 20 | 617 | response to stimulus | BP | fc3.down |
| GO: 0030154 | 0.006723 | 2.127693 | 8.050932 | 16 | 368 | cell differentiation | BP | fc3.down |
| GO: 0008233 | 0.014467 | 2.031607 | 7.274202 | 14 | 347 | peptidase activity | MF | fc3.down |
| GO: 0002520 | 0.029295 | 2.00897 | 5.742448 | 11 | 252 | immune system development | BP | fc3.down |
| GO: 0001568 | 0.038557 | 1.986315 | 5.263911 | 10 | 231 | blood vessel development | BP | fc3.down |
| GO: 0007165 | 0.010716 | 1.622598 | 21.94903 | 33 | 961 | signal transduction | BP | fc3.down |

GOID: Gene Ontology ID
ExpCount: expected count
Count: actual count in data set
Size: size of GO term
Term: GO term Geneset: genes downregulated by DUX4-fl by 3x fold change or more

The up-regulation of a large number of transcription-related and RNA processing factors suggests that DUX4-fl might be a central component of a complex gene regulatory network, and the large number of germline associated genes suggests a possible role in reproductive biology.

In primary human myoblasts, DUX4-fl robustly induced a large number of genes not normally detected in skeletal muscle.

This set of DUX4-fl induced genes, especially ones such as the group of 107 genes (shown in TABLE 2) that were found to be increased by at least 16-fold or greater in the presence of DUX4-fl are useful as biomarkers of DUX4 activity in skeletal muscle, and therefore useful as biomarkers for the presence or risk of developing FSHD, since there would be extremely little to no background expression in control muscle.

GO analysis for these highly induced genes showed enrichment for gamete generation and spermatogenesis categories, as shown in TABLE 6.

TABLE 6
Gene Ontology Analysis of genes upregulated by DUX4-fl 8-fold or more

| GOID | Pvalue | OddsRatio | ExpCount | Count | Size | Term | Ontology | Geneset |
|---|---|---|---|---|---|---|---|---|
| GO: 0042301 | 0.00082 | 58.85380117 | 0.0435084 | 2 | 11 | phosphate binding | MF | fc8 |
| GO: 0051327 | 0.019602 | 10.1148429 | 0.2166402 | 2 | 73 | M phase of meiotic cell cycle | BP | fc8 |
| GO: 0002521 | 0.046737 | 6.215384615 | 0.3472178 | 2 | 117 | leukocyte differentiation | BP | fc8 |
| GO: 0007283 | 0.016709 | 6.116685083 | 0.5460519 | 3 | 184 | spermatogenesis | BP | fc8 |
| GO: 0007276 | 0.033342 | 4.643037975 | 0.7122417 | 3 | 240 | gamete generation | BP | fc8 |
| GO: 0008270 | 9.41E−06 | 4.609476512 | 5.5730248 | 17 | 1409 | zinc ion binding | MF | fc8 |
| GO: 0002682 | 0.043192 | 4.1721673 | 0.7894012 | 3 | 266 | regulation of immune system process | BP | fc8 |
| GO: 0043167 | 7.01E−05 | 3.570087799 | 10.248195 | 22 | 2591 | ion binding | MF | fc8 |

GOID: Gene Ontology ID
ExpCount: expected count
Count: actual count in data set
Size: size of GO term
Term: GO term
Geneset: genes downregulated by DUX4-fl by 3x fold change or more

In many cases, DUX4-fl activated multiple members of gene families involved in germ cell biology and early development, including some primate-specific genes, as shown in Table 7.

TABLE 7
DUX4 highly activates gene families involved in
germ cell and early development

| Gene Family | Members | Biological Context | Fc range |
|---|---|---|---|
| Preferentially | PRAMEF1 | Cancer-testis antigen | 9-269 |
| expressed in | PRAMEF2 | (Chang et al., 2011) | |
| melanoma family | PRAMEF4-15 | | |
| | PRAMEF17 | | |
| | PRAMEF20 | | |
| Tripartite | TRIM43 | Testis-expressed, | 27-235 |
| motif-containing | TRIM48 | preimplantation embryos | |
| | TRIM49 | (Stanghellini et al., 2009) | |
| | TRIM53 | | |
| | TRIM64 | | |
| Methyl-binding | MBD3L2 | Spermatids & germ cell | 197-310 |
| protein-like | MBD3L3 | tumors (Jiang et al., | |
| | MBD3L5 | 2002; | |
| | Jin et al., 2008) | | |
| Zinc finger and | ZSCAN4 | Telomere maintenance in | 13-320 |
| SCAN domain | ZSCAN5B | embryonic stem cells | |
| containing | ZSCAN5D | (Zalzman et al., 2010) | |
| Ret-finger | RFPL1 | Primate neocortex | 20-336 |
| Protein-like | RFPL1S | development | |
| | RFPL2 | (Bonnefont, 2008) | |
| | RFPL3 | | |
| | RFPL4A | | |
| | RFPL4B | | |
| KH homology | KHDC1 | Oocyte- and embryo- | 108-258 |
| domain | KHDC1L | expressed | |
| containing | | (Pierre et al., 2007) | |
| Family with | FAM90A1 | Primate-specific gene | 9-19 |
| sequence | FAM90A2P | family with unknown | |
| similarity 90 | FAM90A6P | function | |
| | FAM90A7 | (Bosch et al., 2007) | |

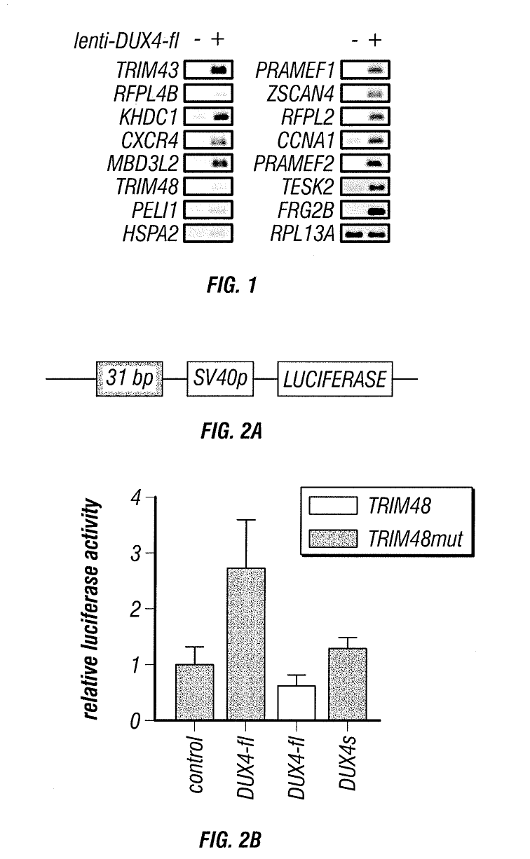

The inventors validated the differential expression of 15 of the DUX4-fl regulated genes by RT-PCR, as shown in FIG. 1. FIG. 1 shows the results of RT-PCR validation of DUX-fl target genes shown to be upregulated in the expression microarray.

### Discussion:

Prior genetic and molecular studies identified DUX4 as the most likely candidate gene for FSHD, however, the abundance of DUX4-fl mRNA was extremely low in FSHD muscle and the protein was not reliably detected. Therefore, it was unclear whether DUX4-fl was expressed at levels sufficient to have a biological consequence in FSHD. The inventors identified genes regulated by DUX4-fl and show that they are expressed at readily detectable levels in FSHD skeletal muscle, both cell lines and muscle biopsies, but not in control tissues, providing direct support for the model that misexpression of DUX4-fl is a causal factor for FSHD. Furthermore, the genes regulated by DUX4-fl suggest several specific mechanisms for FSHD pathophysiology and provide needed candidate biomarkers for the disease.

Currently, the diagnostic test for FSHD1 requires pulse-field gel electrophoresis and Southern blotting to detect the contraction of the D4Z4 repeats, and there are no commercially available diagnostic tests for FSHD2. The set of genes robustly upregulated by DUX4 in FSHD skeletal muscle are candidate biomarkers because they are easily detected in FSHD muscle but absent in control muscle. Furthermore, some target genes encode secreted proteins, such as CSE1L (SEQ ID NO:149) (Genbank NM_{—}177436.1 see Table 1), which suggests the potential for developing blood tests to diagnose FSHD or monitor response to interventions.

Many of the genes highly upregulated by DUX4-fl normally function in the germline and/or early stem cells and are not present in healthy adult skeletal muscle. This supports a biological role for DUX4-fl in germ cell development and suggests potential disease mechanisms for FSHD. Activation of the gametogenic program might be incompatible with post-mitotic skeletal muscle, leading to apoptosis or cellular dysfunction. Also, the testis is an immune-privileged site and testis proteins misexpressed in cancers can induce an immune response (Simpson et al., 2005). In fact, some of the genes regulated by DUX4-fl, such as the PRAME family (Chang et al., 2011), are known cancer testis antigens, so it is reasonable to suggest that expression of these genes in skeletal muscle might also induce an adaptive immune response. An immune-mediated mechanism for FSHD is consistent with the focal inflammation and CD8+ T cell infiltrates that characterize FSHD muscle biopsies (Frisullo et al., 2011; Molnar et al., 1991).

DUX4-fl regulated targets also include genes involved in RNA processing, developmentally regulated components of the PolII transcription complex, ubiquitin-mediated protein degradation pathways and the innate immune response pathways, all of which may have pathophysiological consequences. For example, abnormal splicing has been reported in FSHD, although this was attributed to potential misexpression of another candidate gene for FSHD, FRG1 (Gabellini et al., 2006). In addition, reactivation of retroelements can result in genomic instability (Belancio et al., 2010) and transcriptional deregulation (Schulz et al., 2006), so DUX4's activation of MaLR transcripts might contribute to the apoptosis or modulation of the innate immune response seen in muscle cells expressing DUX4.

### Example 2

This Example describes the identification of DUX4-fl binding sites and a consensus binding sequence motif and demonstrates that DUX4-fl activates the expression of germline genes by binding to a double homeodomain motif.

### Background/Rationale:

Double homeodomain proteins comprise a distinct group of DNA-binding proteins (Holland et al., 2007), but their consensus recognition sites and genomic targets are unknown. Therefore, the inventors performed chromatin immunoprecipitation combined with high throughput sequencing (ChIP-Seq) to identify DUX4-binding sites in human muscle cells, as described in this Example.

### Methods:

### Antibody Development and Characterization

Custom anti-DUX4 polyclonal antibodies MO488 and MO489 were developed through Covance. Rabbits were immunogenized with GST-DUX4 C-terminus fusion protein as antigen (Geng et al., 2011). Human myoblasts were transduced with lentivirus-DUX4-fl and used for testing the antibodies on western blot and immunofluorescence as previously described in Geng et al., 2011, supra. Briefly described, for western blot, 5 μg of lysate from transfected or untransfected cells were run on 4-12% gradient bis-tris polyacrylamide gel and transferred to 0.45 μm nitrocellulose membranes. Membranes were probed with antibodies at a 1:500 dilution. α-tubulin was used as a loading control. Briefly described, for immunofluorescence, cells were fixed in 2% paraformaldehyde and incubated overnight with antibodies at a 1:1000 dilution. Cells were counterstained with 4′,6-diamidino-2-phenylindole (DAPI) for nuclei.

The specificity of the rabbit polyclonal antibodies for DUX4 was confirmed by Western blot and immunofluorescence. In addition, the inventors used the rabbit polyclonals to immunoprecipitate transduced lysates and then demonstrated the pull-down of DUX4 by western blot using a mouse monoclonal antibody to DUX4 (data not shown). Immunoprecipitation of lysates were performed with rabbit polyclonals bound to a 1:1 mixture of Protein A and Protein G Dynabeads (Invitrogen, CA), following manufacturer's instructions. DUX4 protein was immunoprecipitated overnight at 4° C. Precipitated material was eluted directly in Laemmli buffer and boiled for western blot. Samples were run on 4-12% gradient bis-tris polyacrylamide gel, transferred to 0.45 μm nitrocellulose membranes and probed with a custom mouse monoclonal antibody against DUX4 called P4H2. Anti-mouse kappa light chain (SouthernBiotech, Ala.) was used a secondary antibody to minimize cross reactivity against denatured rabbit IgG heavy chain.

It was further determined that the antibodies that were raised against the C-terminus of DUX4-fl do not recognize DUX4-s.

### Immunoprecipitation Analysis

The inventors used two polyclonal rabbit antisera against DUX4 to immunoprecipitate DUX4-fl from human primary myoblasts 24 hours after transduction with lentiviral expressed DUX4-fl or control non-transduced primary myoblasts. Non-redundant reads unambiguously mapped to the human genome were computationally extended to a total length of 200 nucleotides and “peaks” were defined as regions where the number of reads was higher than a statistical threshold compared to the background, as described below. Reads mapping to the X and Y chromosomes were excluded from the analysis.

### Chromatin Immunoprecipitation and Ultra-High-Throughput Sequencing

ChIP was performed and ChIP DNA samples were prepared as previously described in Cao et al., Dev. Cell 18:662-674 (2010), hereby incorporated herein by reference. Anti-DUX4 C-terminus rabbit polyclonal antibodies MO488 and MO489 were combined to immunoprecipitate DUX4-fl. Anti-DUX4 N-terminus polyclonal antibodies FH106 and FH107 were combined to immunoprecipitate DUX4-s. The samples were sequenced with Illumina Genome Analyzer II.

### Defining Peaks

Sequences were extracted by Illumina package GApipeline and reads were aligned using BWA to the human genome (hg18). The inventors only kept one of the duplicated sequences to minimize the artifacts of PCR amplification. Each read was extended in the sequencing orientation to a total of 200 bases to infer the coverage at each genomic position. Peak calling was performed by a house developed R package “peakSig” (pending submission to Bioconductor), which model background reads by a negative binomial distribution. The negative binomial distribution can be viewed as a continuous mixture of Poisson distribution where the mixing distribution of the Poisson rate is modeled as a Gamma prior. This prior distribution is used to capture the variation of background reads density across the genome. Model parameters were estimated by fitting the truncated distribution on the number of bases with low coverage (one to three), to avoid the problem of inferring effective genome size excluding the non-mappable regions, and to eliminate contamination of any foreground signals in the high coverage regions. The inventors also fit a GC dependent mixture model so that the significance of the peaks is determined not only by peak height, but also by the GC content of the neighboring genomic regions.

### Motif Analysis

Discriminative motif discovery was carried out as described in Palii et al. (2011), in which motifs were identified that distinguish a positive and a negative sequence dataset, which in this study, the positive sequences correspond to Dux4 binding sites and negative sequences correspond to the randomly sampled flanking regions of Dux4 binding sites. To generate more accurate presentation of the Dux4 binding sites from the consensus pattern returned by this analysis, the inventors tried to learn a positional weight matrix (PWM) model, using the matches of the consensus pattern as the seed to initialize the iterative expectation-maximization (EM) refinement process similar to MEME. If appropriate, the motifs are extended iteratively as long as there is sequence preference in the flanking region, and refined in the same EM process.

### Electromobility Shift Assay

EMSA was performed with ³²P-labeled 31-bp oligonucleotides from endogenous genomic sequences containing the putative DUX4 binding site as probes (sequences below; only forward shown). Radiolabeled probes were incubated with in vitro translated protein generated from pCS2-DUX4-fl or pCS2-DUX4-s² vectors using the TNT SP6 Coupled Wheat Germ Extract System (Promega) according to manufacturer's instructions. To obtain supershift of protein-DNA complexes, 0.1 μg of E14-3 anti-DUX4 rabbit monoclonal antibody was added to the mixtures. For competition experiments, excess unlabeled probes of either wild-type or mutant sequences were included in the binding reaction. The gels were prepared and run as previously described (Knoepfler et al., 1999).

| | | SEQ ID |
|---|---|---|
| Probe | Forward oligo sequence | NO: |
| TRIM48 | AGGAGTGATGATAATTTAATCAGCCGTGCAA | 150 |
| TRIM48mut | AGGAGTGATGATACTTTTATGAGCCGTGCAA | 151 |
| THED1 | CCTGTGGGAGGTAATCCAATCATGGAGGCAG | 152 |
| THE1Dmut | CCTGTGGGAGGTACTCCTATGATGGAGGCAG | 153 |
| CSF1R | CCAGGTGGAGATAATTGAATCATGGGGGCAG | 154 |
| CSF1Rmut | CCAGGTGGAGATACTTGTATGATGGGGGCAG | 155 |

### Association of Binding and Expression

The inventors associated a peak to its closest TSS within the region flanked by CTCF binding sites, which were identified in a ChIP-seq experiment on human CD4+ T cells (GEO accession number GSE12889/GSM325895).

### Enhancer Activity Reporter Test

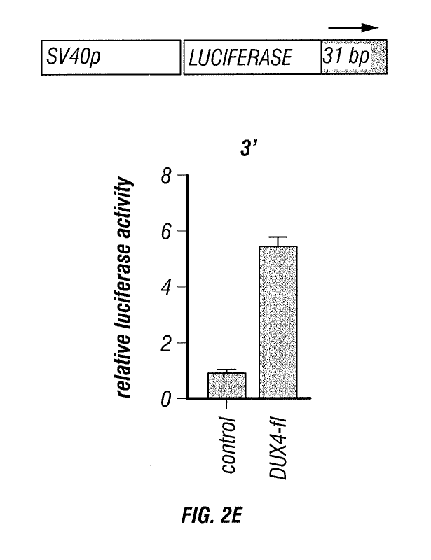

The DUX4 binding site in the ZSCAN4 pGL3-promoter construct was either reversed in orientation (as shown in FIG. 3D) or moved downstream of the reporter gene (as shown in FIG. 3E). Cells were co-transfected with pCS2 expression vectors (1 ug/plate) carrying either β-galactosidase or DUX4-fl and with pGL3-promoter luciferase reporter vectors (1 ug/plate). Transfections and luciferase assays were done as in main methods. Data are given as the averages±SD of triplicates.

### MaLR Expression Analysis

Real-time PCR was performed as described above. Water and minus RT controls were checked to ensure there was no amplification of these repetitive elements from residual or contaminating genomic DNA. Primer sequences were:

(SEQ ID NO: 156)
THE1 forward, 5′-ACCCCTCATGGAGAACCTCT-3′
and
(SEQ ID NO: 157)
THE1 reverse, 5′-ACCCTCTTCTCACAGCTCCA-3′.

### Luciferase Assay

Transient DNA transfections of RD cells were performed using SuperFect (Qiagen) according to manufacturer specifications. Briefly, 3×10⁵ cells were seeded per 35 mm plate the day prior to transfection. Cells were co-transfected with pCS2 expression vectors (2 ug/plate) carrying either β-galactosidase, DUX4-fl or DUX4-s and with pGL3-promoter luciferase reporter vectors (1 μg/plate) carrying various putative DUX4 binding sites or mutant sites upstream of the SV40 promoter or pGL3-basic reporter vector (1 μg/plate) carrying test promoter fragment upstream of the firefly luciferase gene. Cells were lysed 24 h post-transfection in Passive Lysis Buffer (Promega). Luciferase activities were quantified using reagents from the Dual-Luciferase Reporter Assay System (Promega) following manufacturer's instructions. Light emission was measured using BioTek Synergy2 luminometer. Luciferase data are given as the averages±SD of at least triplicates.

### Real-Time PCR of Targets in Matched Testis and Skeletal Muscle

Real-time PCR was performed as described above. Primer sequences for muscle markers are listed below.

| Gene | Forward primer | Reverse primer |
|---|---|---|
| name | sequence | sequence |
| MYH2 | TTCTCAGGCTTCAAGATTTGG | CTGGAGCTTGCGGAATTTAG |
| | (SEQ ID NO: 158) | (SEQ ID NO: 159) |
| CKM | CACCCCAAGTTCGAGGAGAT | AGCGTTGGACACGTCAAATA |
| | (SEQ ID NO: 160) | (SEQ ID NO: 161) |

### DUX4-fl PCR

Nested DUX4-fl3′ PCR on primary myoblast and muscle biopsies were performed as described herein. Primers used were:

182 forward
(SEQ ID NO: 162)
(5′-CACTCCCCTGCGGCCTGCTGCTGGATGA-3′)
and
183 reverse
(SEQ ID NO: 163)
(5′-CCAGGAGATGTAACTCTAATCCAGGTTTGC-3′)
nested with
1A forward
(SEQ ID NO: 164)
(5′-GAG CTC CTG GCG AGC CCG GAG TTT CTG-3′)
and
184 reverse
(SEQ ID NO: 165)
(5′-GTAACTCTAATCCAGGTTTGCCTAGACAGC-3′).

### Knockdown of DUX4-fl Targets

FSHD cultured myoblasts were grown to confluence and switched to differentiation media as described herein. Simultaneously, cells were transduced by lentivirus carrying DUX4-s or GFP along with 8 μg/mL polybrene. Cells were washed and changed to plain differentiation media after 24 hours. Cells were harvested for RNA after 48 hours of differentiation. Untransduced cells were used to assess baseline expression of DUX4-fl target genes.

### Results:

A total of 62,028 and 39,737 peaks were identified at P-value thresholds of 10⁻¹⁰ and 10⁻¹⁵, respectively, after subtracting background peaks in the control samples. DUX4-fl peaks were widely distributed both upstream and downstream of gene transcription start sites (TSSs) with higher numbers in introns and intergenic regions, but showing a relatively constant peak density in all genomic regions when normalized for the size of the genomic compartment. This pattern differs from that reported for many other transcription factors, such as MYOD (Cao et al., 2010), that show higher average peak density in regions near TSSs.

A de novo motif analysis identified the sequence “TAAYBBAATCA” (SEQ ID NO: 166) (IUPAC nomenclature: wherein: T=Thymine; A=Adenine; Y=Pyrimidine (Cytosine (C), Thymine (T), or Uracil (U)); B=Cytosine (C), Thymine (T), Uracil (U) or Guanine (G) (not Adenine (A)); C=cytosine), near the center of greater than 90% of peaks.

To the inventor's knowledge, this motif has not been described for any other transcription factor, but does contain two canonical homeodomain binding motifs (TAAT) arranged in tandem and separated by one nucleotide. Approximately 30% of sequences under the DUX4-fl peaks also contained a second larger motif that encompasses the primary DUX4-fl binding motif. This longer motif matches the long terminal repeat (LTR) of retrotransposons.

Assessment of the representation of DUX4-fl binding at different annotated repetitive elements in the genome shows a nearly 10-fold enrichment of DUX4-fl binding in the Mammalian apparent LTR-Retrotransposon (MaLR) family of retrotransposons and some enrichment in the related ERV family, as shown in TABLE 8 below. Note that the quantitative estimate of repeat-associated binding sites is conservative since reads mapping to more than one locus are excluded from the analysis.

TABLE 8
Repeat families bound by DUX4

| | | Overall | |
|---|---|---|---|
| | DUX4-fl Binding | Genome | DUX4-fl |
| | Prevalence | Prevalence | Enrichment |
| LTR/ERVL-MaLR | 0.35716 | 0.036 | 9.92 |
| LTR/ERV | 0.00032 | 6.00E−05 | 5.33 |
| LTR/ERVK | 0.00803 | 0.0027 | 2.97 |
| LTR/ERVL | 0.04558 | 0.01823 | 2.50 |
| rRNA | 1.00E−04 | 6.00E−05 | 1.67 |
| SINE/tRNA | 0.00011 | 7.00E−05 | 1.57 |
| Unknown | 0.00063 | 0.00043 | 1.47 |
| DNA/TcMar-Mariner | 0.00105 | 0.00092 | 1.14 |
| DNA/TcMar-Tigger | 0.0124 | 0.01121 | 1.11 |
| LTR/Gypsy | 0.00081 | 0.00076 | 1.07 |
| LINE/CR1 | 0.00356 | 0.00356 | 1.00 |
| DNA/hAT? | 0.00016 | 0.00017 | 0.94 |
| LINE/L2 | 0.02978 | 0.03443 | 0.86 |
| SINE/MIR | 0.02317 | 0.0281 | 0.82 |
| LTR/ERV1 | 0.01878 | 0.02604 | 0.72 |
| Satellite | 0.00068 | 0.00103 | 0.66 |
| LINE/L1 | 0.0938 | 0.16059 | 0.58 |
| DNA/hAT-Blackjack | 0.00064 | 0.00113 | 0.57 |
| Simple_repeat | 0.00442 | 0.00836 | 0.53 |
| DNA/hAT-Charlie | 0.00761 | 0.01486 | 0.51 |
| DNA/hAT-Tip100 | 0.00098 | 0.0022 | 0.45 |
| Satellite/centr | 0.00047 | 0.00243 | 0.19 |
| SINE/Alu | 0.00777 | 0.10171 | 0.08 |

DUX4-fl binding prevalence: fraction of all DUX4-fl peaks
Overall genome prevalence: fraction of whole genome
DUX4-fl enrichment: (DUX4-fl binding prevalence)/(overall genome prevalence)

MaLR family members expanded in the primate lineages (Smit, 1993). Thus, if DUX4-fl binding sites were carried throughout the genome during this expansion, these newer sites might have a different sequence motif compared to DUX4-fl binding sites located outside of MaLR repeats. To determine if the expansion of MaLR-associated binding sites might obscure the identification of a different DUX4 binding motif in non-repetitive elements, the inventors performed separate motif analysis of MaLR-associated sites and sites not associated with repeats; both yielded nearly identical core motifs, TAAYYBAATCA (SEQ ID NO:167) and TAAYBYAATCA (SEQ ID NO:168), respectively, but the repeat-associated motifs had slightly more flanking nucleotides preferences reflecting the LTR sequence.

Electrophoretic mobility shift assay (EMSA) confirmed that DUX4-fl binds the core motifs present in both MaLR-associated and non-repeat associated sites: TRIM48 oligos—the TAATTTAATCA (SEQ ID NO:169) core sequence found near the TRIM48 gene, CSF1R oligos—the TAATTGAATCA (SEQ ID NO:171) core sequence found within the LTR of a THE1B retroelement near the CSF1R gene, and THE1D oligos—the TAATCCAATCA (SEQ ID NO:172) core sequence found within the LTR of the THE1D retroelement. Mutation of the core nucleotides abolishes binding, including sites from both repeat and non-repeat regions: competition with cold TRIM48 oligos reduces binding whereas competition with cold TRIM48mut oligos, containing the mutated core sequence TACTTTTATGA (SEQ ID NO:170), does not; competition with the cold CSF1R and THE1D probes to their respective radioactive oligos inhibited binding, whereas competition with cold mutant CSF1Rmut and THE1Dmut oligos, containing sites TACTTCTATG (SEQ ID NO:173) and TACTCCTATGA (SEQ ID NO:174), respectively, do not. Because the DUX4-s alternative splice form retains the N-terminal DNA-binding homeodomains, the inventors hypothesized that it would bind to the same sites as DUX4-fl. EMSA confirmed that DUX4-s specifically binds the same core binding site as DUX4-fl in vitro.

Thus, these results demonstrate that DUX4-s can bind the same sequences as DUX4-fl but does not activate transcription of the same genes, which supports the prior determination that the C-terminus contains a transactivation domain (Kawamura-Saito et al., 2006).

### DUX4-fl is a Transcriptional Activator

The number of DUX4-fl binding locations exceeds the number of genes that robustly increase expression in muscle cells following transduction with DUX4-fl.

A genome-wide analysis of peak height and regional gene expression shows only a weak association of binding and gene expression for DUX4-fl. To determine whether DUX4-fl binding might function as a transcriptional activator at some of the identified binding sites, DUX4 binding sites from selected genes were cloned upstream of the SV40 promoter in the pGL3-promoter luciferase construct as follows (DUX4 binding sites are underlined)

DUX4 binding site from TRIM48:
(SEQ ID NO: 175)
5′ AGGAGTGATGATAATTTAATCAGCCGTGCAA 3′
DUX4 binging site from ZSCAN4:
(SEQ ID NO: 176)
5′ AATCACGTCTTTAAATCAATCACTGACATGG 3′

The 31 bp DUX4 binding site from TRIM48 (SEQ ID NO:175) or ZSCAN4 (SEQ ID NO:176) were inserted into the luciferase reporter construct upstream of the SV40 promoter.

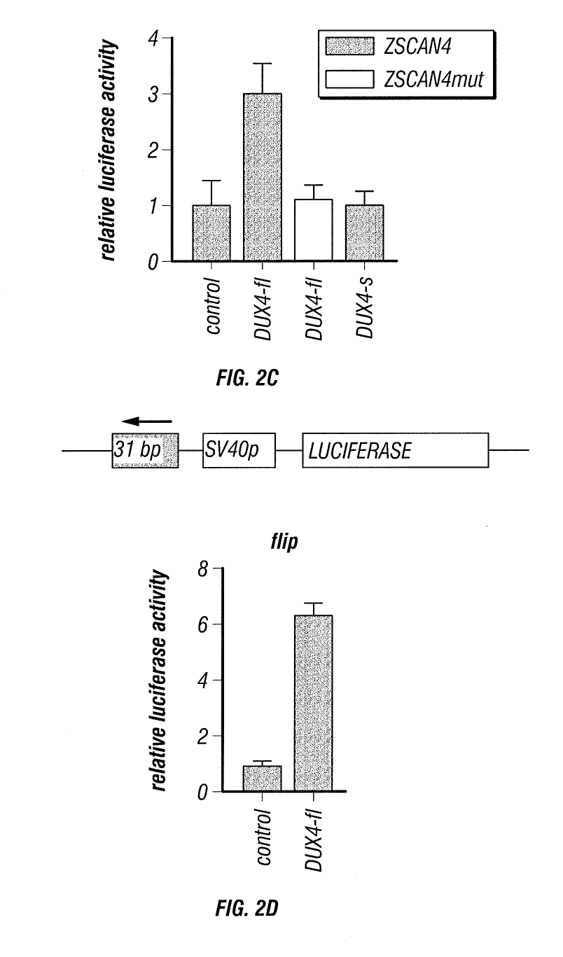

FIG. 2 illustrates that DUX4-fl activates transcription in vivo and DUX4-s can interfere with its activity. FIG. 2A shows reporter construct structure; Genomic fragments near the TRIM48 (FIG. 2B) and ZSCAN4 genes (FIG. 2C) containing DUX4 binding sites were cloned into pGL3-promoter reporter vector (schematic, top) and transfected into human rhabdomyoscaroma cell line RD. Cells were co-transfected with DUX4-fl or DUX4-s. pCS2-13 galactosidase (beta gal) was used to balance DNA amount in control condition. TRIM48mut and ZSCAN4mut are mutated binding sites. Luciferase activity was set relative to control.

As further shown in FIGS. 2D and 2E, DUX4-fl can act as an enhancer at certain loci. FIG. 2D shows the relative luciferase activity in the presence of DUX4-fl from a reporter construct in which the 31 bp DUX4 binding site was inserted in reverse orientation upstream of the SV40 promoter. FIG. 2E shows the relative luciferase activity from a reporter construct in which the 31 bp DUX4 binding site was inserted in the original orientation, but moved downstream of the reporter gene. The luciferase activity was set relative to control plasmid conditions and error bars represent standard deviation of triplicates.

As shown in FIG. 2, co-transfection with DUX4-fl in human rhabdomyosarcoma cell line RD significantly induced luciferase expression independent of orientation or position, and mutation of the DUX4 binding motif eliminated the induction. In contrast to DUX4-fl, DUX4-s did not activate expression despite demonstrating in vitro binding to this site.

To determine whether DUX4 binding might directly regulate transcription of select genes, the inventors cloned the 1.9 kb enhancer and promoter region of the ZSCAN4 gene that includes four DUX4 binding sites as follows: (DUX4 binding sites are underlined)

(SEQ ID NO: 177)
5′AGTAATTCAATCAACAGACAAGTGTTATCCAATCACGTCTTTAAATC
AATCACTGACATGGAGCTGGGGCTGGATGAAGATTCCATCAGTAATTCA
ATCAACAGACAAGTGTTATCCAATCACGTCTTTAAATCAATCACT3′

The 1.9 kb enhancer and promoter region of the ZCAN4 gene that includes the four DUX4 binding sites from ZCAN4 (SEQ ID NO:177) were inserted upstream of the luciferase reporter construct (pGL3 basic luciferase vector). Co-transfection with DUX4-fl significantly induced expression of this reporter and mutation of three of the four DUX4 binding sites nearly abolished the induction. DUX4-s interfered with the activity of DUX4-fl when the two were co-expressed, suggesting that DUX4-s acts as a dominant negative for DUX4-fl activity. DUX4-fl also activated transcription through DUX4 sites in repetitive elements: DUX4-fl activated transcription of a luciferase reporter containing DUX4 binding sites cloned from LTRs at a MaLR THE1D element and RT-PCR showed induction of endogenous MaLR transcripts in muscle cells transduced with DUX4-fl.

### Discussion:

The results in this Example demonstrate that DUX4 binds to and activates transcription from endogenous retrotransposon LTRs of the MaLR family. To the inventor's knowledge, this is the first identification of a transcription factor that can regulate the expression of these repetitive elements in the human genome. The induction of DUX4 expression may be used to induce expression to create placental like invasion and tolerance in allogeneic organ transplants, or to induced mobilization of retrotransposed elements for insertional mutagenesis.

### Example 3

This Example demonstrates that DUX4 targets are normally expressed in human testis but not in healthy skeletal muscle, and that DUX4 regulated genes normally expressed in the testis are aberrantly expressed in FSHD muscle.

### Methods and Materials

### Real Time PCR

One microgram of total RNA was reverse transcribed into first strand cDNA in a 20 uL reaction using SuperScript 111 (Invitrogen) and digested with 1 U of RNase H (Invitrogen) for 20 min at 37° C. cDNA was diluted and used for quantitative PCR with iTaq SYBR Green supermix with ROX (Bio-Rad). The relative expression levels of target genes were normalized to those of ribosomal protein L13A (RPL13A) by 2^{Δct}. Undetermined values were equated to zero. Standard deviations from the mean of the ΔCt values were calculated from triplicates. PCR primers used for detecting the transcripts of the selected genes are listed in Supplementary methods.

### Muscle Biopsies and Human RNA

Muscle biopsy samples were collected from the vastus lateralis muscle of clinically affected and control individuals as previously described (Snider et al., 2010). RNA from matched tissues from healthy donors were purchased from BioChain (Hayward, Calif.).

### Statistical Analyses

Statistical significance between two means was determined by unpaired one-tailed t tests with P-value <0.05. Statistics for the microarray and ChIP-Seq experiments are described separately.

### Results:

### DUX4-fl Directly Regulates Genes Involved in Germline Development

To identify the set of genes that might reflect the function of DUX4-fl prior to the expansion of MaLRs in primates, the inventors identified the subset of genes activated at least 3-fold by DUX4-fl that also contain a non-repeat associated binding site within six kilobases of the TSS and not separated from the TSS by a binding site for the insulator factor CTCF, as shown in TABLE 9.

TABLE 9
Non-repeat element DUX4-fl binding sites associated
with expressed genes

| space | max.cov | Full.fc | Symbol | dist2tss |
|---|---|---|---|---|
| chr22 | 124 | 8.4 | RFPL1 | −3042 |
| chr7 | 114 | 8.3 | hCG_1651160 | −892 |
| chr7 | 215 | 8.3 | hCG_1651160 | −192 |
| chr6 | 232 | 8.3 | RFPL4B | −321 |
| chr19 | 364 | 8.3 | ZSCAN4 | 1430 |
| chr2 | 85 | 8.1 | TRIM43 | −5584 |
| chr2 | 47 | 8.1 | TRIM43 | 1385 |
| chr11 | 178 | 7.9 | TRIM48 | −151 |
| chr1 | 99 | 7.8 | PRAMEF12 | −1230 |
| chr13 | 56 | 7.5 | CCNA1 | −2425 |
| chr13 | 58 | 7.5 | CCNA1 | 1874 |
| chr22 | 118 | 7.3 | RFPL2 | −2853 |
| chr22 | 129 | 7.3 | RFPL2 | 3057 |
| chr14 | 344 | 7 | PNP | −104 |
| chr11 | 85 | 6.9 | TRIM49L1 | 200 |
| chr11 | 109 | 6.9 | TRIM49L2 | −202 |
| chr8 | 55 | 6.4 | DEFB103A | −2289 |
| chr19 | 110 | 5.5 | ZNF296 | 182 |
| chr11 | 373 | 4.2 | SFRS2B | 42 |
| chr5 | 91 | 4.1 | PPP2R2B | 2498 |
| chr20 | 55 | 4.1 | ZNF217 | −2168 |
| chr20 | 122 | 4.1 | ZNF217 | 3546 |
| chr12 | 224 | 3.9 | ZNF705A | −5106 |
| chr22 | 118 | 3.8 | PANX2 | −2040 |
| chr19 | 98 | 3.8 | ZSCAN5B | −5014 |
| chr19 | 67 | 3.8 | ZSCAN5B | −4101 |
| chr19 | 89 | 3.8 | ZSCAN5B | 4892 |
| chr16 | 118 | 3.7 | SIAH1 | −2337 |
| chr12 | 129 | 3.6 | FAM90A1 | −1597 |
| chr12 | 54 | 3.6 | PRR4 | 83 |
| chr3 | 117 | 3.4 | DBR1 | 3981 |
| chr11 | 105 | 3.3 | SPTY2D1 | −797 |
| chr11 | 74 | 3.3 | SPTY2D1 | 5934 |
| chr14 | 117 | 3.2 | FBXO33 | −2226 |
| chr19 | 83 | 3.2 | GTF2F1 | 134 |
| chr17 | 177 | 3.2 | JUP | −701 |
| chr22 | 234 | 3.2 | TFIP11 | −1353 |
| chr21 | 96 | 3.1 | CLDN14 | −2523 |
| chr20 | 53 | 2.9 | CSE1L | −5914 |
| chr20 | 57 | 2.9 | CSE1L | −1935 |
| chr2 | 85 | 2.9 | PELI1 | 4936 |
| chr7 | 63 | 2.7 | BZW2 | 1939 |
| chr7 | 86 | 2.7 | BZW2 | 1955 |
| chr10 | 265 | 2.7 | CCNJ | −637 |
| chr1 | 116 | 2.6 | DENND2C | −3169 |
| chr14 | 257 | 2.6 | PABPN1 | −529 |
| chr7 | 128 | 2.6 | SRRT | 1546 |
| chr19 | 104 | 2.6 | USP29 | 2090 |
| chr19 | 66 | 2.6 | USP29 | 3676 |
| chr14 | 159 | 2.5 | C14orf102 | −558 |
| chr11 | 46 | 2.4 | CTR9 | 1913 |
| chr21 | 229 | 2.4 | SYNJ1 | −766 |
| chr6 | 159 | 2.3 | NFYA | 49 |
| chr1 | 132 | 2.2 | C1orf63 | −1326 |
| chr17 | 283 | 2.2 | HOXB2 | −2471 |
| chr3 | 51 | 2.2 | PVRL3 | 2678 |
| chr6 | 250 | 2.1 | C6orf191 | 61 |
| chr10 | 70 | 2.1 | CBARA1 | 2340 |
| chr10 | 250 | 2.1 | CBARA1 | 4786 |
| chr21 | 244 | 2.1 | SON | −2951 |
| chr10 | 56 | 2 | AVPI1 | 126 |
| chr10 | 200 | 2 | FRG2B | 598 |
| chr16 | 46 | 2 | RBBP6 | −4040 |
| chr16 | 323 | 2 | RBBP6 | −1922 |
| chr16 | 76 | 2 | RBBP6 | −1619 |
| chr1 | 139 | 1.9 | EXOSC10 | 150 |
| chr2 | 150 | 1.9 | GPBAR1 | −224 |
| chr9 | 85 | 1.9 | NANS | 1843 |
| chr20 | 62 | 1.9 | SNAI1 | 769 |
| chr18 | 65 | 1.9 | TAF4B | −1741 |
| chr21 | 274 | 1.8 | C21orf91 | −803 |
| chr2 | 163 | 1.8 | CLK1 | 4634 |
| chr1 | 93 | 1.8 | KDM5B | 301 |
| chr12 | 73 | 1.8 | KIF21A | −4249 |
| chr12 | 37 | 1.8 | KIF21A | −1030 |
| chr17 | 43 | 1.8 | MED13 | −2755 |
| chr10 | 172 | 1.8 | SEC61A2 | −1861 |
| chr11 | 133 | 1.8 | SPRYD5 | −2855 |
| chr14 | 50 | 1.7 | C14orf138 | −4696 |
| chr14 | 53 | 1.7 | C14orf138 | 326 |
| chr12 | 55 | 1.7 | DDX47 | −4416 |
| chr5 | 269 | 1.7 | MAST4 | −1253 |
| chr10 | 57 | 1.7 | PRPF18 | 3230 |
| chr6 | 105 | 1.7 | PTP4A1 | −2227 |
| chr9 | 198 | 1.6 | CTNNAL1 | 641 |
| chr1 | 205 | 1.6 | EGLN1 | −1 |
| chr9 | 69 | 1.6 | MAPKAP1 | −4397 |
| chr9 | 153 | 1.6 | MAPKAP1 | 3619 |
| chr2 | 52 | 1.6 | RTN4 | −2562 |
| chr2 | 115 | 1.6 | RTN4 | 1853 |
| chr10 | 43 | 1.6 | SAMD8 | 3338 |
| chr9 | 88 | 1.6 | SH3GL2 | −4600 |

Chromosome: chromosome location of binding site
max.cov: peak height
full.fc: expression fold change for DUX4-fl targets
dist2tss: distance to TSS

The 74 genes meeting these criteria are highly enriched for genes involved in stem and germ cell functions, RNA processing, and regulated components of the PolII complex, similar to the major GO categories identified for all of the genes regulated by DUX4-fl. Quantitative RT-PCR of six DUX4 regulated genes (PRAMEF1; RFPL2; TRIM43; ZSCAN4; KHDC1; MBD3L2) on paired samples of testis mRNA and skeletal muscle mRNA from two control individuals found high expression of these targets in the testes and absent, or nearly absent, expression in skeletal muscle, supporting a conserved role for DUX4 in germline biology. The inventors also detected the expression of the related DUXA and DUX1 genes in healthy testis (data not shown), further supporting the notion that this family of double homeodomain proteins has a role in germ cell biology. The results of real-time RT-qPCR analysis of gene expression in human testis versus matched skeletal muscle tissue from two healthy donors showed that DUX4 targets tested are normally expressed in human testis but not in healthy skeletal muscle. The expression results shown are presented relative to internal standard RPL13a, and error bars represent standard deviation of PCR triplicates for the following DUX-fl target genes: (A) PRAMEF1; (B) RFPL2; (C) TRIM43; (D) ZSCAN4; (E) KHDC1; (F) MBD3L2; and controls (G) MYH2 (skeletal muscle marker), (H) CKM (skeletal muscle marker), and (I) RPL13a.

DUX4-fl-Regulated Gene Targets are Expressed in FSHD Muscle

To determine whether the low levels of endogenous DUX4-fl mRNA detected in FSHD skeletal muscle is sufficient to activate DUX4 target genes, the inventors assessed the expression of some of these genes ((A) PRAMEF1; (B) RFPL2; (C) MBD3L2; (D) TRIM43; (E) KHDC1; and (F) ZSCAN4) in a set of control and FSHD muscle. Cultured muscle cells from control biopsies showed low or absent expression of the six DUX4-fl regulated genes, whereas these genes were expressed at significantly higher levels in the FSHD muscle cultures, including those from both FSHD1 and FSHD2 individuals.

The endogenous DUX4-fl expression status is provided in TABLE 10.

TABLE 10
DUX4-fl expression in FSHD and control muscle

| | | | |
|---|---|---|---|
| Sample # | Formal Identifier | DUX4-fl expression | Disease Status |

Primary Human Myoblasts

| | | | |
|---|---|---|---|
| 1 | MB135 | not detected | control |
| 2 | MB196 | not detected | control |
| 3 | MB201 | not detected | control |
| 4 | MB209 | not detected | control |
| 5 | MB230 | not detected | control |
| 6 | MB54-1* | not detected | control* |
| 7 | MB073 | detected | FSHD1 |
| 8 | MB183 | detected | FSHD1 |
| 9 | MB197 | detected | FSHD1 |
| 10 | MB216 | detected | FSHD1 |
| 11 | MB200 | detected | FSHD2 |
| 12 | MB54-2* | detected | FSHD1* |

Muscle Biopsies

| | | | |
|---|---|---|---|
| 1 | C-20 | not detected | control |
| 2 | C-22 | not detected | control |
| 3 | C-33 | not detected | control |
| 4 | C-38 | not detected | control |
| 5 | C-40 | not detected | control |
| 6 | C-2333/C-2397 | not detected | control |
| 7 | F-2315 | not detected | FSHD1 |
| 8 | F-2316 | detected | FSHD1 |
| 9 | F-2319 | not detected | FSHD1 |
| 10 | F-2326 | not detected | FSHD1 |
| 11 | F-2331 | detected | FSHD1 |
| 12 | F-2367 | detected | FSHD1 |
| 13 | F-2369 | detected | FSHD1 |
| 14 | F-2377 | detected | FSHD1 |

*Myoblasts cultured from the same mosaic individual that either do not have (MB54-1) or have (MB54-2) a contracted 4q allele

The results obtained demonstrate that DUX4 regulated genes normally expressed in the testis are aberrantly expressed in FSHD muscle. Similar to the expression of DUX4-fl regulated targets in cultured FSHD muscle, muscle biopsies from FSHD individuals had readily detectable mRNA of DUX4-fl regulated genes, although at varying levels in different biopsies.

For the results of real-time RT-PCR analysis of expression of DUX4-fl target genes in Control and FSHD muscle biopsies from 15 individuals for the following target genes: (A) PRAMEF1; (B) RFPL2; (C) MBD3L2; (D) TRIM43; (E) KHDC1; and (F) ZSCAN4, it is noted that the DUX4-fl mRNA is at extremely low abundance in FSHD muscle and it is notable that some biopsy samples in which the DUX4-fl mRNA was not detected showed elevation of DUX4 regulated targets (Table 10), indicating that the target mRNA is of significantly higher abundance and perhaps more stable than the DUX4 mRNA. The DUX4 expression status in the muscle samples in Control and FSHD muscle biopsies from 15 individuals was analyzed, as determined by nested DUX4 PCR on cDNA from cultured muscle cells or biopsies. RPL13A PCR was used for an internal standard. The coded sample names and complete status information for the biopsy samples are provided in TABLE 10.

To determine whether the expression of the DUX4 target genes in FSHD muscle was due to binding of the DUX4 protein to its consensus DNA motif, the inventors used DUX4-s to interfere with DUX4-fl activity. As shown above, DUX4-s binds the same consensus motif as DUX4-fl but does not activate gene expression and co-transfection of DUX4-s with DUX4-fl interferes with the ability of DUX4-fl to activate a reporter construct.

Lentiviral expression of DUX4-s in FSHD muscle cells inhibited the endogenous expression of the target genes as well, indicating that the DUX4 target genes in FSHD muscle require an activating factor that binds at the DUX4 motif, which is most likely the DUX4-fl protein. DUX4-s blocks expression of DUX4-fl target genes in FSHD muscle cells. DUX4-s maintains the DNA binding domain of DUX4 but lacks the transcriptional activation domain and therefore acts as a dominant negative to DUX4-fl by binding to the DUX4 motif (see FIG. 2). Real-time RT-PCR quantitation of three DUX4 target genes, (A) PRAMEF1, (B) RFPL2 and (C) MBD3L2 in FSHD cultured muscle cells transduced with lenti-GFP or lenti-DUX4-s or untransduced was performed.

In summary, this data support the model that inappropriate expression of DUX4 plays a causal role in FSHD skeletal muscle pathophysiology by activating germline gene expression and endogenous retrotransposons in postmitotic skeletal muscle. Beyond their utilities as candidate biomarkers, the DUX4 targets identified in Example 1 point to specific mechanisms of disease and may help guide the development of therapies for FSHD.

### Example 4

This Example demonstrates that DUX4-fl activates expression of multiple cancer testis antigens and gene families in FSHD muscle and DUX4-fl expression correlates with expression of cancer testis antigens (CTAs) in a cancer cell and CTA family members are induced by DUX4-fl in dendritic cells.

### Methods and Results:

### DUX4-fl Activates Expression of Cancer Testis Antigens and Gene Families in FSHD Muscle

As described above in EXAMPLES 1-3, based on expression array data, many of the genes found to be activated by DUX4-fl in skeletal muscle cells are expressed in the germline, and some are close family members of cancer testis antigens. For example, DUX4-fl activates the expression of CSAG3 and PRAMEF1, as well as other PRAME family members, whereas CSAG2 and PRAME have been characterized as inducing a T-cell response to cancers.

To further analyze the expression of cancer testis antigen in the presence of DUX4-fl, an experiment was carried out in which normal skeletal muscle cells were transduced with the Lentiviral vector expressing either DUX4-fl, or a control Lentiviral vector expressing GFP, generated as described in Example 1, and the transduced cells were analyzed by RT-PCR for expression of several known cancer testis antigens. The results were normalized to an internal control standard of either 18S or GAPDH.

Seven known cancer testis antigens: BAGE, MAGEA4, MAGEA9, SSX1, SSX2, SSX4, and one of the PRAME family members are all induced over 2-fold by the expression of DUX4-fl as shown by RT-PCR analysis.

To determine whether the T-cell infiltrate associated with FSHD represents an oligoclonal response to a disease-related antigen, the T-cell receptor beta-chain was deep-sequenced from DNA isolated from a muscle biopsy from an FSHD patient. Three independent regions of the biopsy showed clonal expansion of the same small number of T-cell clones, demonstrating a clonal expansion consistent with the response to a limited set of antigens, in which a dominant clone was found to be present, representing over one million of the sequences and a small number of other clones. In contrast, deep sequencing of T-cell receptors from a control peripheral blood showed a broad representation of different T-cell receptors, the most abundant present at about 100,000 times (data not shown). Therefore, the T-cells infiltrating the FSHD skeletal muscle represent a small number of clones that have expanded, consistent with an immune response to a muscle expressed antigen.

DUX4-fl Expression Correlates with Expression of Cancer Testis Antigens (CTAs) in a Cancer Cell and CTA Family Members are Induced by DUX4-fl in Dendritic Cells

It was determined that DUX4-fl is expressed in some cancer cell lines and its expression correlates with expression of CTAs. It was further determined that the colon cancer cell line HCT116 does not express DUX4-fl but DUX4-fl expression is induced when the two major DNA methyltransferase genes are disrupted in the HCT116 double knock-out line. The results of an RT-PCR assay for DUX4-fl in HCT116 cells demonstrated that DNA methylation suppresses DUX4 expression. DUX4-fl is not detected in the parental HCT116 colon cancer cell line (wt lane), nor in the derivatives of this line that have single gene knock-outs for DNMT1 (1−/− lane) or DNMT3b (3b−/− lane); however, DUX4-fl is expressed in the double knock-out of DNMT1 and DNMT3b (DKO lane) that substantially reduces the degree of DNA CpG methylation. xxx

In addition, DUX4-fl expression can be detected in HCT116 cells treated with the demethylating agent azacytidine (decitabine). Treatment of HCT116 cells with the DNA demethylating agent 5-azacytidine (decitabine) induces the expression of DUX4-fl, as determined by RT-PCR for DUX4-fl in different tumor cell lines.

FIG. 3 is a Heat map showing expression of cancer testis antigens (CTA) in HCT116 cells under conditions that activate DUX4-fl expression (i.e., treatment with the demethylating agent 5-azacytidine). The relative expression of the CTA in each row was measured by RT-PCR and represented as high (light shading) or low (dark shading). The first column shows very low expression of CTAs in HCT116 that are not treated (−) and the second column shows a robust induction after treatment with azacytidine (+), a condition that induces expression of DUX4-fl. Similar patterns are seen with the DNMT1 and DNMT3 mutants, whereas a higher basal level of CTAs is seen in the DKO even before azacytidine treatment. Therefore, there is a strong correlation between expression of DUX4-fl and the expression of CTAs. As further shown in FIG. 3, the expression of DUX4-fl in these cells correlates with upregulation of multiple known CTAs, including BAGE, NYESO1 and MAGE and PRAME family members.

An experiment was carried out to measure the expression level of DUX4-fl regulated genes in chronic myelogenous leukemia (CML) cells using an RT-PCR assay for the target genes. The results showed that DUX4-fl regulated genes are expressed in CML cells that express the PRAME cancer testis antigen (CTA). It is noted that CML1 does not express the PRAME CTA, whereas CML2 and CML3 do express the PRAME CTA. CML2 and CML3 also express genes regulated by DUX4-fl, TRIM43 and ZSCAN4, indicating that DUX4-fl or a DUX family member is likely activating these genes in the CML cells. In CML cells, the expression of the PRAME CTA correlates with the expression of other DUX4-fl targets, indicating that DUX4-fl or another member of the DUX family, is likely the transcriptional driver of CTA expression in these cells.

### Expression of DUX4-fl in HCT116 Cells Induces the Expression of the CTA Family Member PRAMEF1.

Expression of DUX4-fl in HCT116, primary dendritic cells, and in two cancer cell lines JJ and FS shows robust activation of the PRAMEF1 gene, a gene highly related to the PRAME CTA. The cancer cell lines JJ and FS are tumor cell lines derived from patients with chondrosarcomas, as described in Jagasia et al. (1996).

Results showed that expression of DUX4-fl in HCT116 cells induces the expression of the CTA family member PRAMEF1 in HCT116 wt cells. HCT116 cells were infected with a control lenti-GFP or with lenti-DUX4-fl and the abundance of PRAMEF1 mRNA measured relative to a constitutively expressed gene, RPL13a.

Results showed that expression of DUX4-fl induces expression of the CTA family member PRAMEF1 in primary dendritic cells and cancer cell lines. Primary dendritic cells and the tumor cell lines derived from patients with chondrosarcomas designated JJ and FS (described in Jagasia, et al., 1996, supra) were transduced with lentiviral constructs expressing a control gene, green fluorescent protein (GFP), or DUX4fl. PRAMEF1 mRNA was measured by real-time PCR relative to a constitutively expressed control, RPL13a.

### Summary of Results:

These results demonstrate that DUX4-fl activates expression of multiple cancer testis antigens and gene families in FSHD muscle and DUX4-fl expression correlates with expression of cancer testis antigens (CTAs) in a cancer cell and CTA family members are induced by DUX4-fl in dendritic cells.

### Example 5

This Example demonstrates that DUX4-fl inhibits the innate immune response induced by lenti-viral infection and further that DUX4-fl induces expression of a secreted factor that suppresses the innate immune response.

### Methods and Results:

### DUX4-fl Inhibits the Innate Immune Response Induced by Lenti-Viral Infection.

In the experiment generating expression array data described in Example 1, the inventors surprisingly determined that lentiviral infection activates the innate immune response in human muscle cells, whereas DUX4-fl suppresses the induction of the innate immune response. In this experiment, Human myoblasts were infected with control lenti-virus expressing green fluorescent protein (lenti-GFP), DUX4-fl (lenti-DUX4-fl), or the short splice form of DUX4 that lacks the carboxyterminal region of the protein (lenti-DUX4-s). As shown previously, DUX4-s contains the DNA binding domains but lacks the carboxyterminal activation domain, and therefore binds DNA but does not activate gene transcription. RNA was harvested from the lenti-viral infected cells and uninfected control cells at 24 hours after infection.

Compared to uninfected control cells, the lenti-GFP infection induced expression of 341 genes using a 2-fold change and FDR<0.01 criteria, as shown in TABLE 11.

TABLE 11
Genes Induced by Lentiviral Constructs in Human Muscle Cells

| | Symbol | GFP/NoLenti.fc | Full/GFP.fc | Short/GFP.fc | Full/NoLenti.fc | Short/NoLenti.fc |
|---|---|---|---|---|---|---|
| 1 | IFI27 | 7.30 | −6.53 | 0.27 | 0.92 | 7.60 |
| 2 | MX1 | 7.21 | −5.67 | 0.26 | 1.58 | 7.49 |
| 3 | IFITM1 | 6.73 | −6.12 | 0.34 | 0.73 | 7.08 |
| 4 | IFI44L | 5.75 | −5.12 | 0.47 | 0.74 | 6.22 |
| 5 | CFB | 5.39 | −5.62 | 0.63 | 0.07 | 6.04 |
| 6 | HERC5 | 5.37 | −3.20 | 1.33 | 2.21 | 6.71 |
| 7 | SOD2 | 5.21 | −4.62 | 1.53 | 0.67 | 6.75 |
| 8 | IFI6 | 5.09 | −4.24 | −0.04 | 0.82 | 5.05 |
| 9 | ISG15 | 5.06 | −4.31 | 0.13 | 0.74 | 5.20 |
| 10 | IFIT1 | 4.79 | −4.38 | 0.72 | 0.43 | 5.50 |
| 11 | BST2 | 4.69 | −4.25 | 0.21 | 0.58 | 4.91 |
| 12 | OAS1 | 4.38 | −4.61 | 1.45 | 0.13 | 5.82 |
| 13 | IFIT2 | 4.37 | −4.31 | 2.07 | 0.20 | 6.44 |
| 14 | EPSTI1 | 4.36 | −4.12 | 0.66 | 0.34 | 5.05 |
| 15 | LOC100129681 | 4.24 | −4.07 | 0.52 | 0.34 | 4.75 |
| 16 | SERPINA3 | 4.12 | −2.22 | −0.29 | 1.89 | 3.83 |
| 17 | STAT1 | 3.95 | −2.92 | 0.82 | 1.08 | 4.79 |
| 18 | HERC6 | 3.89 | −3.73 | 0.52 | 0.32 | 4.41 |
| 19 | MX2 | 3.85 | −4.07 | 0.96 | 0.07 | 4.80 |
| 20 | SAA1 | 3.85 | −4.14 | 0.26 | 0.03 | 4.11 |
| 21 | TNFAIP6 | 3.80 | −2.21 | 1.31 | 1.63 | 5.10 |
| 22 | CCL20 | 3.73 | 0.11 | 1.63 | 3.84 | 5.35 |
| 23 | OAS3 | 3.73 | −4.03 | 0.87 | −0.09 | 4.59 |
| 24 | IFIH1 | 3.69 | −3.70 | 1.66 | 0.25 | 5.34 |
| 25 | LY6E | 3.69 | −3.25 | 0.25 | 0.44 | 3.94 |
| 26 | IFIT3 | 3.68 | −3.86 | 1.81 | 0.04 | 5.49 |
| 27 | ECGF1 | 3.59 | −4.30 | 0.62 | −0.66 | 4.21 |
| 28 | IL8 | 3.52 | −3.88 | 1.19 | −0.13 | 4.69 |
| 29 | C1QTNF1 | 3.51 | −3.85 | 0.77 | −0.12 | 4.28 |
| 30 | HLA-B | 3.45 | −2.75 | 0.34 | 0.70 | 3.78 |
| 31 | C1R | 3.43 | −2.83 | 0.49 | 0.67 | 3.92 |
| 32 | IFI35 | 3.41 | −3.85 | 0.92 | −0.32 | 4.33 |
| 33 | IFI44 | 3.29 | −2.42 | 0.29 | 0.90 | 3.59 |
| 34 | CXCL1 | 3.16 | −3.52 | 0.20 | −0.04 | 3.36 |
| 35 | SLC15A3 | 3.13 | −3.75 | 0.78 | −0.51 | 3.91 |
| 36 | PRIC285 | 3.01 | −2.61 | 0.86 | 0.45 | 3.87 |
| 37 | SAMD9 | 2.93 | −2.86 | 1.40 | 0.13 | 4.32 |
| 38 | CHI3L2 | 2.88 | −2.70 | 0.26 | 0.43 | 3.14 |
| 39 | FOS | 2.87 | 1.08 | 0.47 | 3.94 | 3.33 |
| 40 | IRF7 | 2.86 | −1.96 | 1.14 | 1.02 | 3.99 |
| 41 | PARP12 | 2.85 | −3.17 | 0.78 | −0.17 | 3.62 |
| 42 | VWCE | 2.84 | −3.32 | −0.12 | −0.22 | 2.72 |
| 43 | EIF2AK2 | 2.72 | −2.30 | 0.54 | 0.45 | 3.27 |
| 44 | MT1M | 2.70 | −2.92 | 1.19 | −0.17 | 3.90 |
| 45 | LGALS3BP | 2.68 | −2.12 | 0.31 | 0.57 | 3.00 |
| 46 | VCAM1 | 2.68 | −3.01 | 0.63 | −0.11 | 3.31 |
| 47 | XAF1 | 2.66 | −3.62 | 0.62 | −0.83 | 3.27 |
| 48 | AGRN | 2.63 | −2.61 | 0.38 | 0.10 | 3.01 |
| 49 | TMEM140 | 2.61 | −2.95 | 1.00 | −0.03 | 3.59 |
| 50 | PARP14 | 2.58 | −2.83 | 0.52 | −0.09 | 3.10 |
| 51 | FBXO32 | 2.57 | −1.18 | 0.77 | 1.41 | 3.33 |
| 52 | S1PR3 | 2.56 | −2.12 | 1.13 | 0.53 | 3.68 |
| 53 | TAP1 | 2.55 | −3.26 | 0.70 | −0.70 | 3.26 |
| 54 | SP110 | 2.52 | −2.94 | 1.15 | −0.33 | 3.66 |
| 55 | NAMPT | 2.51 | −0.58 | 1.21 | 1.95 | 3.72 |
| 56 | HLA-E | 2.49 | −1.11 | 0.81 | 1.37 | 3.30 |
| 57 | CCL5 | 2.47 | −2.53 | 1.89 | 0.22 | 4.33 |
| 58 | HIST2H2AA3 | 2.46 | 1.29 | 1.07 | 3.74 | 3.52 |
| 59 | PSMB9 | 2.43 | −3.14 | 0.95 | −0.53 | 3.37 |
| 60 | IRF9 | 2.43 | −2.25 | 0.29 | 0.20 | 2.72 |
| 61 | CCL2 | 2.39 | −2.68 | 0.44 | 0.01 | 2.82 |
| 62 | OAS2 | 2.38 | −2.71 | 0.46 | 0.00 | 2.82 |
| 63 | SAMD9L | 2.37 | −2.53 | 1.26 | −0.04 | 3.62 |
| 64 | CD68 | 2.37 | −1.09 | 1.18 | 1.28 | 3.54 |
| 65 | DHX58 | 2.35 | −2.84 | 0.70 | −0.17 | 3.04 |
| 66 | USP18 | 2.32 | −2.50 | 1.00 | 0.09 | 3.29 |
| 67 | ISG20 | 2.30 | −2.94 | 2.36 | −0.38 | 4.63 |
| 68 | KIAA0247 | 2.30 | −2.13 | 0.37 | 0.29 | 2.66 |
| 69 | ABCA1 | 2.29 | −1.21 | −0.32 | 1.10 | 1.97 |
| 70 | UBE2L6 | 2.26 | −3.08 | 0.58 | −0.83 | 2.84 |
| 71 | PTX3 | 2.26 | −2.74 | 0.94 | −0.18 | 3.19 |
| 72 | SLC7A2 | 2.26 | −2.48 | −0.11 | −0.13 | 2.15 |
| 73 | RARRES3 | 2.25 | −2.22 | 1.27 | 0.20 | 3.51 |
| 74 | HIST2H2AA4 | 2.24 | 1.31 | 1.07 | 3.54 | 3.30 |
| 75 | TRIM22 | 2.24 | −2.58 | −0.02 | −0.26 | 2.22 |
| 76 | DDR2 | 2.23 | −2.22 | 0.63 | 0.05 | 2.86 |
| 77 | TNFAIP3 | 2.22 | −2.90 | 0.28 | −0.38 | 2.50 |
| 78 | IGFBP4 | 2.22 | −1.94 | 0.40 | 0.30 | 2.64 |
| 79 | GBP2 | 2.19 | −2.40 | 0.66 | −0.19 | 2.87 |
| 80 | C10orf10 | 2.19 | −2.64 | 0.14 | −0.32 | 2.33 |
| 81 | NFKBIA | 2.19 | −3.00 | 0.28 | −0.83 | 2.46 |
| 82 | TRIM25 | 2.18 | −2.51 | 0.51 | −0.27 | 2.69 |
| 83 | STOM | 2.17 | −2.29 | 0.62 | −0.10 | 2.80 |
| 84 | PARP9 | 2.13 | −2.10 | 0.44 | 0.15 | 2.56 |
| 85 | DDX58 | 2.12 | −2.47 | 0.02 | −0.18 | 2.14 |
| 86 | SP100 | 2.10 | −2.28 | 1.22 | 0.08 | 3.29 |
| 87 | DKK1 | 2.06 | −2.29 | 0.47 | −0.24 | 2.53 |
| 88 | MIR1978 | 2.06 | −1.28 | 0.09 | 0.79 | 2.14 |
| 89 | RSAD2 | 2.05 | −2.40 | 2.36 | −0.06 | 4.38 |
| 90 | HLA-C | 2.04 | −1.79 | 0.60 | 0.32 | 2.65 |
| 91 | CEBPD | 2.03 | −1.52 | 0.16 | 0.49 | 2.19 |
| 92 | IL18BP | 2.01 | −1.25 | 1.40 | 0.85 | 3.37 |
| 93 | | 2.00 | −2.51 | −0.13 | −0.25 | 1.88 |
| 94 | SUSD2 | 2.00 | −1.40 | −2.02 | 0.69 | 0.15 |
| 95 | IFITM3 | 1.98 | −1.95 | −0.13 | −0.01 | 1.84 |
| 96 | MT1X | 1.98 | −2.44 | 0.32 | −0.47 | 2.29 |
| 97 | SHISA5 | 1.98 | −1.76 | −0.27 | 0.22 | 1.69 |
| 98 | MSI2 | 1.97 | −1.76 | 1.33 | 0.28 | 3.29 |
| 99 | ZBTB16 | 1.97 | −2.04 | 0.49 | 0.11 | 2.45 |
| 100 | XPC | 1.95 | −2.46 | 0.26 | −0.48 | 2.21 |
| 101 | SPATA18 | 1.94 | −1.99 | −0.21 | 0.08 | 1.73 |
| 102 | TRIM21 | 1.94 | −1.36 | 0.98 | 0.63 | 2.91 |
| 103 | SESN1 | 1.94 | −1.58 | −0.11 | 0.40 | 1.83 |
| 104 | UGCG | 1.93 | −0.89 | 1.45 | 1.04 | 3.37 |
| 105 | STAT2 | 1.92 | −2.69 | 0.19 | −0.77 | 2.11 |
| 106 | RTP4 | 1.88 | −2.28 | 1.21 | −0.09 | 3.05 |
| 107 | FST | 1.88 | −1.81 | 0.91 | 0.08 | 2.79 |
| 108 | HLA-F | 1.87 | −1.93 | 0.94 | 0.14 | 2.79 |
| 109 | DDX60 | 1.86 | −2.35 | 0.45 | −0.26 | 2.30 |
| 110 | NFKBIZ | 1.85 | −1.57 | −0.26 | 0.30 | 1.58 |
| 111 | NFIL3 | 1.84 | −1.82 | 0.32 | 0.07 | 2.16 |
| 112 | IFI16 | 1.84 | −2.01 | 0.68 | −0.14 | 2.52 |
| 113 | APCDD1 | 1.82 | −1.89 | −0.43 | 0.11 | 1.41 |
| 114 | CXCL5 | 1.80 | −2.47 | 0.82 | −0.37 | 2.60 |
| 115 | DCN | 1.78 | −1.67 | 0.26 | 0.22 | 2.04 |
| 116 | TAPBP | 1.78 | −1.84 | 0.47 | −0.04 | 2.25 |
| 117 | CMBL | 1.78 | −1.99 | 0.07 | −0.18 | 1.85 |
| 118 | PAPPA | 1.77 | −2.26 | 0.40 | −0.26 | 2.16 |
| 119 | GRINA | 1.77 | −1.43 | 0.47 | 0.37 | 2.24 |
| 120 | GDF15 | 1.77 | −2.17 | 0.92 | −0.28 | 2.69 |
| 121 | LNPEP | 1.76 | −1.35 | 2.08 | 0.48 | 3.82 |
| 122 | ZNFX1 | 1.75 | −2.13 | 0.82 | −0.35 | 2.56 |
| 123 | LAP3 | 1.75 | −1.55 | 1.40 | 0.21 | 3.16 |
| 124 | PSMB8 | 1.74 | −2.25 | 0.71 | −0.39 | 2.45 |
| 125 | MAMDC2 | 1.73 | −1.86 | −0.03 | −0.11 | 1.71 |
| 126 | GFPT2 | 1.73 | −1.61 | 0.05 | 0.14 | 1.78 |
| 127 | UBA7 | 1.72 | −2.56 | 0.57 | −0.69 | 2.29 |
| 128 | SLC2A5 | 1.72 | −1.96 | 0.81 | −0.10 | 2.52 |
| 129 | SLC44A1 | 1.71 | −1.13 | 0.78 | 0.61 | 2.49 |
| 130 | C19orf66 | 1.71 | −2.35 | 0.62 | −0.46 | 2.32 |
| 131 | SERPING1 | 1.69 | −1.67 | 1.21 | 0.23 | 2.87 |
| 132 | STXBP6 | 1.69 | −2.23 | −0.14 | −0.52 | 1.54 |
| 133 | HIST1H2AC | 1.68 | 0.26 | 1.07 | 1.92 | 2.70 |
| 134 | TSC22D3 | 1.68 | −1.73 | 0.20 | 0.03 | 1.87 |
| 135 | PARP10 | 1.67 | −2.06 | 0.54 | −0.13 | 2.19 |
| 136 | COL7A1 | 1.67 | −2.12 | −0.19 | −0.41 | 1.49 |
| 137 | ZC3H12A | 1.66 | −1.55 | 0.54 | 0.29 | 2.18 |
| 138 | HIPK3 | 1.66 | −0.30 | 1.55 | 1.39 | 3.15 |
| 139 | GBP1 | 1.66 | −2.24 | 1.01 | −0.54 | 2.66 |
| 140 | LOC729009 | 1.66 | −2.65 | 0.66 | −1.00 | 2.32 |
| 141 | TNFSF13B | 1.65 | −1.76 | 2.19 | 0.12 | 3.78 |
| 142 | TGFBR3 | 1.64 | −1.34 | 0.46 | 0.43 | 2.08 |
| 143 | CABC1 | 1.64 | −1.49 | −0.18 | 0.22 | 1.47 |
| 144 | PLEKHA4 | 1.64 | −1.46 | 1.33 | 0.24 | 2.95 |
| 145 | NDRG1 | 1.63 | −2.33 | 0.66 | −0.66 | 2.29 |
| 146 | GALNTL2 | 1.63 | −1.83 | 0.99 | 0.03 | 2.58 |
| 147 | PDK4 | 1.62 | −1.52 | 0.03 | 0.29 | 1.64 |
| 148 | ERAP2 | 1.62 | −1.61 | 0.77 | 0.05 | 2.38 |
| 149 | CXCL6 | 1.61 | −1.99 | 0.17 | −0.10 | 1.77 |
| 150 | LOC387763 | 1.60 | −1.46 | 0.20 | 0.32 | 1.80 |
| 151 | CYP27A1 | 1.60 | −1.60 | −0.06 | 0.04 | 1.54 |
| 152 | FTHL3 | 1.58 | −2.58 | 0.45 | −0.97 | 2.04 |
| 153 | PHF11 | 1.57 | −2.27 | 0.66 | −0.66 | 2.22 |
| 154 | CYBASC3 | 1.57 | −1.95 | −0.42 | −0.35 | 1.15 |
| 155 | MLKL | 1.57 | −2.99 | 1.46 | −1.15 | 3.01 |
| 156 | CYP26B1 | 1.56 | −1.46 | 0.41 | 0.20 | 1.96 |
| 157 | ZNF650 | 1.55 | −1.02 | 0.69 | 0.59 | 2.23 |
| 158 | FUCA1 | 1.55 | −1.61 | 0.29 | 0.02 | 1.84 |
| 159 | C9orf169 | 1.55 | −2.51 | −0.05 | −0.79 | 1.50 |
| 160 | RORA | 1.55 | 0.89 | 1.89 | 2.40 | 3.38 |
| 161 | DUSP19 | 1.52 | −2.38 | 0.75 | −0.84 | 2.28 |
| 162 | EVC | 1.51 | −1.95 | −0.39 | −0.37 | 1.14 |
| 163 | IL7R | 1.51 | −2.26 | 0.57 | −0.49 | 2.05 |
| 164 | CA12 | 1.51 | −2.63 | 0.95 | −0.85 | 2.44 |
| 165 | FOXQ1 | 1.49 | −1.43 | 0.64 | 0.27 | 2.07 |
| 166 | PSME1 | 1.49 | −1.14 | 0.28 | 0.33 | 1.76 |
| 167 | PCTK3 | 1.48 | −1.64 | 0.59 | 0.07 | 2.03 |
| 168 | HIST2H2AC | 1.47 | 1.31 | 0.96 | 2.77 | 2.42 |
| 169 | CFD | 1.46 | −1.20 | 0.25 | 0.37 | 1.70 |
| 170 | C4orf34 | 1.46 | −1.04 | 0.61 | 0.42 | 2.06 |
| 171 | SGK | 1.45 | 2.04 | −0.11 | 3.50 | 1.34 |
| 172 | PDPN | 1.45 | −1.62 | −0.29 | −0.09 | 1.17 |
| 173 | C18orf56 | 1.45 | −1.62 | 0.21 | −0.06 | 1.66 |
| 174 | PTGFR | 1.45 | −1.09 | 0.15 | 0.45 | 1.59 |
| 175 | SERPINE2 | 1.44 | −1.37 | 0.20 | 0.07 | 1.65 |
| 176 | AHR | 1.44 | 1.50 | 1.64 | 2.90 | 3.04 |
| 177 | MUC1 | 1.43 | −1.74 | 0.35 | −0.14 | 1.77 |
| 178 | RN7SK | 1.43 | 1.06 | 0.47 | 2.42 | 1.86 |
| 179 | LOC643384 | 1.42 | −2.22 | 1.02 | −0.76 | 2.43 |
| 180 | RTN1 | 1.42 | −1.35 | −0.06 | 0.23 | 1.37 |
| 181 | MAOA | 1.42 | −1.71 | −0.38 | −0.08 | 1.07 |
| 182 | MYBPHL | 1.41 | −1.49 | −0.52 | 0.02 | 0.91 |
| 183 | SPPL2A | 1.41 | −0.75 | 1.12 | 0.66 | 2.52 |
| 184 | ANPEP | 1.40 | −2.12 | 0.36 | −0.69 | 1.76 |
| 185 | LOC389386 | 1.40 | −1.52 | 1.37 | −0.06 | 2.76 |
| 186 | BTN3A2 | 1.40 | −1.76 | 0.34 | −0.18 | 1.72 |
| 187 | CENTG2 | 1.39 | −1.31 | 0.52 | 0.12 | 1.91 |
| 188 | NT5C3 | 1.39 | −0.66 | 1.88 | 0.73 | 3.26 |
| 189 | CA9 | 1.38 | −1.53 | 0.52 | 0.07 | 1.87 |
| 190 | KRT17 | 1.38 | −1.63 | 0.46 | −0.03 | 1.81 |
| 191 | OSBPL8 | 1.37 | 1.95 | 1.05 | 3.27 | 2.39 |
| 192 | C4orf18 | 1.37 | −1.78 | −0.51 | −0.38 | 0.86 |
| 193 | TP53INP1 | 1.37 | −1.14 | −0.57 | 0.26 | 0.81 |
| 194 | ADAR | 1.36 | −2.34 | 0.31 | −0.96 | 1.68 |
| 195 | APOBEC3G | 1.36 | −2.01 | 1.41 | −0.39 | 2.72 |
| 196 | IRAK3 | 1.35 | −1.50 | 1.05 | 0.01 | 2.37 |
| 197 | CST3 | 1.35 | −1.13 | 0.16 | 0.24 | 1.53 |
| 198 | C13orf15 | 1.35 | 1.35 | −0.26 | 2.63 | 1.12 |
| 199 | RRM2B | 1.35 | −1.01 | 0.62 | 0.42 | 1.95 |
| 200 | CCND2 | 1.35 | −1.06 | −0.53 | 0.30 | 0.82 |
| 201 | BTN3A3 | 1.34 | −1.62 | 0.40 | −0.05 | 1.72 |
| 202 | EEA1 | 1.34 | −0.96 | 1.03 | 0.50 | 2.31 |
| 203 | RIOK3 | 1.34 | −0.52 | 1.13 | 0.81 | 2.46 |
| 204 | GBP4 | 1.34 | −1.48 | 2.34 | 0.08 | 3.58 |
| 205 | PSME2 | 1.33 | −1.48 | 0.43 | −0.13 | 1.78 |
| 206 | MTSS1 | 1.33 | −1.16 | −0.41 | 0.26 | 0.94 |
| 207 | RELB | 1.33 | −1.39 | 0.29 | 0.13 | 1.59 |
| 208 | MUSK | 1.32 | −1.35 | 0.62 | 0.14 | 1.91 |
| 209 | IL1R1 | 1.32 | −1.34 | 0.30 | 0.09 | 1.61 |
| 210 | CEBPB | 1.32 | −2.44 | 0.28 | −1.13 | 1.59 |
| 211 | TNFRSF6B | 1.31 | −1.68 | 0.01 | −0.29 | 1.32 |
| 212 | CSF3 | 1.31 | −1.63 | 0.81 | −0.07 | 2.06 |
| 213 | ARID4B | 1.30 | 1.40 | 0.57 | 2.69 | 1.86 |
| 214 | HLA-H | 1.30 | −1.80 | 0.33 | −0.37 | 1.62 |
| 215 | CIDEC | 1.30 | −1.31 | −0.03 | 0.17 | 1.27 |
| 216 | MT1G | 1.29 | −1.27 | 0.25 | 0.05 | 1.55 |
| 217 | FTHL11 | 1.29 | −2.09 | 0.60 | −0.77 | 1.89 |
| 218 | IFITM2 | 1.29 | −1.71 | 0.01 | −0.46 | 1.29 |
| 219 | RBM43 | 1.29 | −1.52 | 0.32 | −0.07 | 1.59 |
| 220 | ABI3BP | 1.29 | −1.27 | −0.41 | 0.04 | 0.89 |
| 221 | MMP7 | 1.28 | −1.27 | 0.21 | 0.22 | 1.48 |
| 222 | C6orf138 | 1.28 | −1.31 | 0.98 | 0.11 | 2.23 |
| 223 | NFE2L2 | 1.28 | −1.09 | 0.33 | 0.20 | 1.61 |
| 224 | HIPK2 | 1.27 | −1.17 | 0.36 | 0.12 | 1.63 |
| 225 | FRMD3 | 1.26 | −1.12 | −0.29 | 0.26 | 1.00 |
| 226 | ARFGEF2 | 1.26 | 0.13 | 1.34 | 1.38 | 2.54 |
| 227 | C14orf159 | 1.26 | −2.07 | 0.52 | −0.70 | 1.77 |
| 228 | OASL | 1.25 | −1.33 | 1.74 | 0.13 | 2.88 |
| 229 | GAS1 | 1.25 | −1.73 | 0.01 | −0.48 | 1.26 |
| 230 | HCG4 | 1.25 | −1.65 | 0.68 | −0.38 | 1.92 |
| 231 | YPEL3 | 1.25 | −1.28 | −0.27 | 0.01 | 0.98 |
| 232 | SLC39A8 | 1.24 | −1.22 | 0.90 | 0.15 | 2.11 |
| 233 | CYGB | 1.24 | −1.40 | 0.16 | 0.02 | 1.39 |
| 234 | BTG2 | 1.24 | −1.57 | −0.12 | −0.28 | 1.12 |
| 235 | CLDN15 | 1.24 | −1.55 | −0.37 | −0.07 | 0.91 |
| 236 | BCL6 | 1.24 | −1.26 | 0.36 | −0.01 | 1.60 |
| 237 | MMP3 | 1.24 | −1.51 | 0.28 | −0.05 | 1.50 |
| 238 | EGFR | 1.24 | −1.39 | 1.21 | −0.06 | 2.42 |
| 239 | UNC93B1 | 1.23 | −1.96 | 0.94 | −0.64 | 2.17 |
| 240 | MT1F | 1.22 | −1.18 | −0.54 | 0.08 | 0.70 |
| 241 | HLA-A | 1.22 | −1.08 | 0.56 | 0.15 | 1.80 |
| 242 | TP53I3 | 1.22 | −1.96 | 0.19 | −0.72 | 1.41 |
| 243 | HECW2 | 1.22 | −1.17 | 0.06 | 0.17 | 1.27 |
| 244 | LOC653879 | 1.22 | −1.42 | 0.33 | 0.02 | 1.51 |
| 245 | IGFBP5 | 1.22 | −1.44 | 0.18 | −0.07 | 1.38 |
| 246 | SLC22A18 | 1.21 | −1.56 | −0.05 | −0.23 | 1.16 |
| 247 | FILIP1L | 1.21 | −1.78 | 0.07 | −0.51 | 1.28 |
| 248 | TNFRSF14 | 1.21 | −2.72 | 0.57 | −1.36 | 1.78 |
| 249 | CES2 | 1.21 | −2.04 | 0.06 | −0.80 | 1.27 |
| 250 | H1F0 | 1.21 | −2.43 | 0.50 | −1.15 | 1.71 |
| 251 | C1RL | 1.21 | −1.63 | −0.16 | −0.24 | 1.06 |
| 252 | PPAP2A | 1.21 | −1.11 | 0.54 | 0.13 | 1.75 |
| 253 | RNU6-15 | 1.21 | 1.01 | 0.71 | 2.22 | 1.91 |
| 254 | HIST2H2BE | 1.21 | 1.35 | 0.27 | 2.51 | 1.46 |
| 255 | SSH2 | 1.20 | −1.28 | −0.32 | −0.05 | 0.89 |
| 256 | DNAJC3 | 1.19 | 0.87 | 1.60 | 2.02 | 2.74 |
| 257 | MR1 | 1.19 | −1.28 | 0.68 | 0.01 | 1.85 |
| 258 | SPTLC3 | 1.19 | −1.44 | 0.24 | −0.11 | 1.42 |
| 259 | TCEA3 | 1.19 | −1.71 | −0.12 | −0.50 | 1.07 |
| 260 | JUNB | 1.19 | −1.06 | 0.52 | 0.23 | 1.67 |
| 261 | NACC2 | 1.18 | −1.79 | 0.98 | −0.43 | 2.14 |
| 262 | PHLDA3 | 1.18 | −1.56 | 0.20 | −0.34 | 1.38 |
| 263 | TTC39B | 1.18 | −1.26 | 1.23 | 0.03 | 2.37 |
| 264 | SCHIP1 | 1.18 | −2.09 | 0.06 | −0.81 | 1.24 |
| 265 | CFLAR | 1.17 | −1.94 | 0.53 | −0.70 | 1.71 |
| 266 | ATL3 | 1.17 | −1.46 | 1.35 | −0.26 | 2.51 |
| 267 | ACSM5 | 1.17 | −1.34 | 0.05 | −0.04 | 1.22 |
| 268 | DRAM1 | 1.17 | −1.09 | −0.36 | 0.11 | 0.81 |
| 269 | LTBR | 1.17 | −1.15 | 0.41 | 0.02 | 1.57 |
| 270 | SUSD1 | 1.17 | −1.30 | 0.51 | −0.05 | 1.67 |
| 271 | FTH1 | 1.17 | −2.03 | −0.42 | −0.84 | 0.76 |
| 272 | SLC7A11 | 1.17 | −1.36 | 1.35 | 0.03 | 2.43 |
| 273 | DDX60L | 1.17 | −1.64 | 0.66 | −0.29 | 1.80 |
| 274 | CORO6 | 1.15 | −1.12 | −0.30 | 0.06 | 0.86 |
| 275 | UGP2 | 1.15 | −1.17 | 0.11 | 0.02 | 1.26 |
| 276 | LUM | 1.15 | −1.13 | 0.39 | 0.04 | 1.54 |
| 277 | NDUFA4L2 | 1.15 | −1.27 | 0.19 | 0.03 | 1.33 |
| 278 | PTGES | 1.15 | −1.63 | 0.31 | −0.26 | 1.44 |
| 279 | DGKA | 1.15 | −1.96 | 0.10 | −0.59 | 1.24 |
| 280 | C1S | 1.14 | −1.19 | 0.42 | 0.16 | 1.52 |
| 281 | TAP2 | 1.14 | −1.25 | 1.35 | 0.04 | 2.44 |
| 282 | CABYR | 1.13 | −1.76 | −0.04 | −0.46 | 1.09 |
| 283 | MOCOS | 1.13 | −1.72 | 0.42 | −0.53 | 1.55 |
| 284 | ALDH3A2 | 1.13 | −1.28 | −0.20 | −0.12 | 0.93 |
| 285 | FTHL8 | 1.12 | −2.35 | 0.51 | −1.23 | 1.64 |
| 286 | KYNU | 1.12 | −1.31 | 1.46 | 0.03 | 2.46 |
| 287 | NRCAM | 1.11 | −1.30 | −0.11 | 0.00 | 1.01 |
| 288 | PYGB | 1.11 | −2.05 | −0.12 | −0.91 | 0.99 |
| 289 | ZFHX3 | 1.11 | −1.76 | 0.50 | −0.59 | 1.61 |
| 290 | ITPRIP | 1.10 | −1.76 | 0.55 | −0.61 | 1.66 |
| 291 | ASAM | 1.10 | −1.26 | 0.02 | −0.04 | 1.12 |
| 292 | MTE | 1.10 | −2.38 | 0.50 | −1.25 | 1.60 |
| 293 | SLC39A14 | 1.10 | −1.87 | 0.48 | −0.65 | 1.57 |
| 294 | STK17B | 1.09 | −0.06 | 1.31 | 1.03 | 2.39 |
| 295 | PSTPIP2 | 1.09 | −1.38 | 0.48 | −0.09 | 1.53 |
| 296 | CXCL2 | 1.09 | −1.34 | 0.00 | −0.04 | 1.09 |
| 297 | MME | 1.09 | −1.05 | −0.18 | 0.05 | 0.90 |
| 298 | SEMA4B | 1.09 | −1.94 | 0.15 | −0.66 | 1.23 |
| 299 | COPS8 | 1.08 | −1.26 | 0.14 | −0.15 | 1.23 |
| 300 | HLA-G | 1.08 | −1.36 | 0.12 | −0.12 | 1.19 |
| 301 | TDRD7 | 1.08 | −1.06 | 1.07 | 0.11 | 2.12 |
| 302 | SLC30A1 | 1.08 | −0.35 | 1.24 | 0.76 | 2.27 |
| 303 | BCL3 | 1.08 | −1.23 | −0.04 | −0.12 | 1.03 |
| 304 | SRGN | 1.08 | −1.87 | 0.20 | −0.80 | 1.28 |
| 305 | LOC100133866 | 1.07 | −1.34 | −0.13 | −0.18 | 0.95 |
| 306 | TNFSF10 | 1.07 | −1.13 | 2.97 | 0.14 | 3.88 |
| 307 | AK3 | 1.07 | −1.26 | −0.43 | −0.18 | 0.65 |
| 308 | IFIT5 | 1.07 | −1.65 | −0.28 | −0.36 | 0.81 |
| 309 | NCOA7 | 1.07 | 1.31 | −0.06 | 2.36 | 1.01 |
| 310 | PDE4B | 1.07 | −1.14 | 0.68 | 0.11 | 1.68 |
| 311 | DDB2 | 1.07 | −1.41 | 0.11 | −0.16 | 1.16 |
| 312 | FKBP5 | 1.06 | −1.76 | −0.10 | −0.71 | 0.95 |
| 313 | LEPR | 1.06 | −1.00 | −0.55 | 0.10 | 0.53 |
| 314 | APOOL | 1.06 | −0.58 | 1.37 | 0.53 | 2.39 |
| 315 | BATF2 | 1.05 | −1.28 | 1.38 | −0.02 | 2.32 |
| 316 | FLT3LG | 1.05 | −1.07 | 0.39 | 0.13 | 1.40 |
| 317 | FBXO6 | 1.05 | 0.03 | 1.02 | 1.08 | 1.99 |
| 318 | IDS | 1.04 | −1.02 | 0.67 | 0.05 | 1.69 |
| 319 | SLU7 | 1.04 | 1.51 | 0.72 | 2.53 | 1.74 |
| 320 | HIST1H4H | 1.04 | 1.03 | 1.23 | 1.97 | 2.16 |
| 321 | PCBP3 | 1.04 | −1.12 | 0.21 | 0.02 | 1.24 |
| 322 | SAA2 | 1.04 | −1.22 | 0.28 | 0.02 | 1.29 |
| 323 | ANKRA2 | 1.04 | −1.26 | 0.34 | −0.14 | 1.36 |
| 324 | C3 | 1.03 | −1.00 | 0.25 | 0.21 | 1.25 |
| 325 | STC1 | 1.03 | −1.57 | 0.81 | −0.43 | 1.82 |
| 326 | TNFRSF10A | 1.03 | −1.13 | 0.84 | 0.07 | 1.81 |
| 327 | KIAA1618 | 1.03 | −1.84 | −0.30 | −0.72 | 0.74 |
| 328 | KLF9 | 1.02 | −1.26 | −0.05 | −0.22 | 0.97 |
| 329 | PLXNB1 | 1.02 | −1.44 | −0.10 | −0.34 | 0.93 |
| 330 | CASP1 | 1.02 | −1.17 | 2.17 | 0.05 | 3.02 |
| 331 | PLA2G4C | 1.01 | −1.43 | 0.07 | −0.29 | 1.09 |
| 332 | LOC644423 | 1.01 | −1.35 | 0.00 | −0.23 | 1.02 |
| 333 | TRIM55 | 1.01 | −1.05 | 0.20 | −0.04 | 1.22 |
| 334 | TNFAIP2 | 1.01 | −1.59 | 0.06 | −0.42 | 1.06 |
| 335 | SSBP2 | 1.01 | −1.29 | 0.35 | −0.26 | 1.36 |
| 336 | RND3 | 1.01 | 1.39 | 0.93 | 2.37 | 1.92 |
| 337 | TLR3 | 1.00 | −1.27 | 1.67 | −0.06 | 2.56 |
| 338 | UBR1 | 1.00 | −0.09 | 1.11 | 0.92 | 2.03 |
| 339 | C1orf57 | 1.00 | −1.12 | 0.35 | −0.09 | 1.36 |
| 340 | HOXC13 | 1.00 | −1.11 | 0.44 | −0.09 | 1.45 |
| 341 | PTGER2 | 1.00 | −1.18 | −0.20 | −0.18 | 0.80 |

As shown in TABLE 11, of these 341 genes induced by lenti-GFP infection, 315 were induced 2-fold or more by the lenti-DUX4-s infection. The majority of these genes are known to be in the innate immune response pathway and are likely to be induced by the introduction of the lenti-viral RNA. However, of the 341 genes induced by lenti-GFP, only 34 were induced two-fold or more by DUX4-fl and only 24 were induced within 50% of the level of induction by the lenti-GFP. Therefore, these results show that the expression of DUX4-fl suppresses the induction of 317 out of 341 (93%) lentivirus-induced genes.

Further in this regard, it is noted that DUX4-fl suppresses the induction of the three primary sensors of viral RNA (LGP2 (DHX58), IFIH1 (MDA5), and DDX58 (RIG-1)), which both positively activate their own transcription and also activate the transcription of additional transcription factors, such as IRF1 and IRF7, as shown in FIG. 2 of Sandling et al., (2011), hereby incorporated herein by reference. With continued reference to FIG. 2 of Sandling, et al., it is further noted that DUX4-fl suppressed the induction of these additional specific genes was observed (TNFAIP3, TBK1, NFKB activation, IKBKE, IRF1, IRF7, TLR3, STAT2, STAT1, IRF9, IL8, CXCL10, TNFSF138). It was further noted that DUX4-fl suppressed the induction of the following close homologues to the genes shown in FIG. 2 of Sandling, et al. (TRAM, TRIF, IFNAR2, TNFSF4).

As shown below in TABLE 12, both IRF1 and IRF7 induction is suppressed by DUX4-fl, as well as multiple components of the downstream pathway, including NFKB, interferons, STATS, TNF family members and cytokines.

TABLE 12
Representative Genes Induced by Lenti-GFP and Lenti-DUX4-s but
poorly induced by Lenti-DUX4-fl (log2 fold change: GFP = control-lenti;
Full = DUX4-fl, Short = DUX4-s; NoLenti = uninfected)

| | | | | | | |
|---|---|---|---|---|---|---|
| | Genbank Ref | | | | | |
| Symbol | No.* | GFP/NoLenti.fc | Full/GFP.fc | Short/GFP.fc | Full/NoLenti.fc | Short/NoLenti.fc |

Immediate Responders

| | | | | | | |
|---|---|---|---|---|---|---|
| IFIH1 | NM_022168.2 | 3.69 | −3.70 | 1.66 | 0.25 | 5.34 |
| (MDA5) | | | | | | |
| DDX58 | NM_014314.3 | 2.12 | −2.47 | 0.02 | −0.18 | 2.14 |
| DHX58 | NM_024119.2 | 2.35 | −2.84 | 0.70 | −0.17 | 3.04 |
| (LGP2) | | | | | | |

Representative genes in the Pathway

| | | | | | | |
|---|---|---|---|---|---|---|
| TNFAIP3 | NM_006290.2 | 2.22 | −2.90 | 0.28 | −0.38 | 2.50 |
| TNFAIP6 | NM_007115.2 | 3.80 | −2.21 | 1.31 | 1.63 | 5.10 |
| TNFSF13B | NM_006573.3 | 1.65 | −1.76 | 2.19 | 0.12 | 3.78 |
| TNFRSF6B | NM_032945.2 | 1.31 | −1.68 | 0.01 | −0.29 | 1.32 |
| TNFRSF14 | NM_003820.2 | 1.21 | −2.72 | 0.57 | −1.36 | 1.78 |
| TNFSF10 | NM_003810.2 | 1.07 | −1.13 | 2.97 | 0.14 | 3.88 |
| TNFRSF10A | NM_003844.2 | 1.03 | −1.13 | 0.84 | 0.07 | 1.81 |
| TNFAIP2 | NM_006291.2 | 1.01 | −1.59 | 0.06 | −0.42 | 1.06 |
| IRF7 | NM_004029.2 | 2.86 | −1.96 | 1.14 | 1.02 | 3.99 |
| IRF9 | NM_006084.4 | 2.43 | −2.25 | 0.29 | 0.20 | 2.72 |
| NFKBIA | NM_020529.1 | 2.19 | −3.00 | 0.28 | −0.83 | 2.46 |
| NFKBIZ | NM_001005474.1 | 1.85 | −1.57 | −0.26 | 0.30 | 1.58 |
| IL7R | NM_002185.2 | 1.51 | −2.26 | 0.57 | −0.49 | 2.05 |
| IL8 | NM_000584.2 | 3.52 | −3.88 | 1.19 | −0.13 | 4.69 |
| IL18BP | NM_173042.2 | 2.01 | −1.25 | 1.40 | 0.85 | 3.37 |
| NFIL3 | NM_005384.2 | 1.84 | −1.82 | 0.32 | 0.07 | 2.16 |
| IL1R1 | NM_000877.2 | 1.32 | −1.34 | 0.30 | 0.09 | 1.61 |
| CXCL1 | NM_001511.1 | 3.16 | −3.52 | 0.20 | −0.04 | 3.36 |
| CXCL5 | NM_002994.3 | 1.80 | −2.47 | 0.82 | −0.37 | 2.60 |
| CXCL6 | NM_002993.2 | 1.61 | −1.99 | 0.17 | −0.10 | 1.77 |
| CXCL2 | NM_002089.3 | 1.09 | −1.34 | 0.00 | −0.04 | 1.09 |
| STAT1 | NM_007315.2 | 3.95 | −2.92 | 0.82 | 1.08 | 4.79 |
| STAT2 | NM_005419.2 | 1.92 | −2.69 | 0.19 | −0.77 | 2.11 |
| TLR3 | NM_003265.2 | 1.00 | −1.27 | 1.67 | −0.06 | 2.56 |
| IFI27 | NM_005532.3 | 7.30 | −6.53 | 0.27 | 0.92 | 7.60 |
| IFITM1 | NM_003641.3 | 6.73 | −6.12 | 0.34 | 0.73 | 7.08 |
| IFI44L | NM_006820.1 | 5.75 | −5.12 | 0.47 | 0.74 | 6.22 |
| MX1 | NM_002462.2 | 7.21 | −5.67 | 0.26 | 1.58 | 7.49 |
| IFITM1 | NM_003641.3 | 6.73 | −6.12 | 0.34 | 0.73 | 7.08 |
| IFI44L | NM_006820.1 | 5.75 | −5.12 | 0.47 | 0.74 | 6.22 |
| IFI6 | NM_022872.2 | 5.09 | −4.24 | −0.04 | 0.82 | 5.05 |
| IFIT1 | NM_001548.3 | 4.79 | −4.38 | 0.72 | 0.43 | 5.50 |
| IFIT3 | NM_001549.2 | 3.68 | −3.86 | 1.81 | 0.04 | 5.49 |
| IFI35 | NM_005533.2 | 3.41 | −3.85 | 0.92 | −0.32 | 4.33 |
| IFI44 | NM_006417.3 | 3.29 | −2.42 | 0.29 | 0.90 | 3.59 |
| CCL5 | NM_002985.2 | 2.47 | −2.53 | 1.89 | 0.22 | 4.33 |
| CCL2 | NM_002982.3 | 2.39 | −2.68 | 0.44 | 0.01 | 2.82 |
| IFITM3 | NM_021034.2 | 1.98 | −1.95 | −0.13 | −0.01 | 1.84 |
| IFI16 | NM_005531.1 | 1.84 | −2.01 | 0.68 | −0.14 | 2.52 |
| IFITM2 | NM_006435.2 | 1.29 | −1.71 | 0.01 | −0.46 | 1.29 |
| IFIT5 | NM_012420.1 | 1.07 | −1.65 | −0.28 | −0.36 | 0.81 |

GFP/NoLenti, Expression ratio of lenti-GFP infected vs not infected;
Full/GFP.fc, expression ratio of lenti-DUX4-fl to lenti-GFP
Short/GFP.fc, Expression ratio of lenti-DUX4-s to lenti-GFP;
Full/NoLenti.fc, expression ratio of lenti-DUX4-fl to no infection;
Short/NoLenti.fc, Expression ratio of lenti-DUX4-s to no infection.
*The sequence of each Genbank No. referenced in TABLE 12 is hereby incorporated by reference herein, with reference to Genbank accessed on Jul. 27, 2011.

Because GFP and DUX4-s do not have any significant sequence similarities on the RNA or protein level, the inventors concluded that the common set of genes activated by lenti-GFP and lenti-DUX4-s represent a response to the viral RNA that activates a common innate immune response pathway. Because DUX4-fl differs from DUX4-s primarily by lacking a transcriptional activation domain, the inventors further concluded that transcriptional activation of a gene or genes by DUX4-fl suppresses the innate immune response.

DUX4-fl Induces Expression of a Secreted Factor that Suppresses the Innate Immune Response

It was determined that DEFB103A (SEQ ID NO:49) and DEFB103B (SEQ ID NO:107), each encoding the polypeptide DEFB103A/B set forth as SEQ ID NO:178, are both activated by lenti-DUX4-fl and not by lenti-DUX4-s or lenti-GFP. These represent transcripts from a duplicated beta-defensin 3 gene, which can function as a suppressor of inflammation. As a secreted protein, DEFB103A/B (SEQ ID NO:178) has the potential to suppress the innate immune response in cells infected with lenti-GFP. As an initial step toward testing this, the inventors transduced cells with lenti-DUX4-fl or nothing for 12 hrs, washed extensively and replaced the culture media, then after an additional 12 hrs transferred the conditioned media to cells infected 24 hrs earlier with lenti-GFP.

Results of a RT-PCR assay showed that conditioned media from lenti-DUX4-fl transduced cells suppresses the induction of IFIH1 by lenti-GFP transduced cells. Cultured human muscle cells (54-1) were transduced with nothing (first lane panels A and B), lenti-DUX4-fl @ approximately MOI=10 (lane 2), lenti-DUX4-fl MOI ˜1 (lane 3), lenti-GFP MOI ˜10 (lane 4), lenti-GFP transduced cells exposed to conditioned media from lane 2 cells (lane 5), lenti-GFP transduced cells exposed to conditioned media from lane 3 cells (lane 6), lenti-GFP transduced cells exposed to media from lane 5 cells that was filtered through a 35 kD cut-off filter. The conditioned media from the lenti-DUX4 transduced cells suppresses the IFIH1 induction by lenti-GFP (Panel A). Filtering through a 35 kD filter does not remove all of the factor(s) responsible for the suppression. Lenti-DUX4-fl induces expression of DEFB103A/B, whereas the conditioned media does not, nor does lenti-GFP.

Thus, in cells infected without exposure to DUX4-fl conditioned medium, qRT-PCR confirmed that lenti-GFP activated IFIH1 and lenti-DUS4-fl did not; whereas lenti-GFP did not activate DEFB103A/B and DUX4-fl did. Conditioned media from lenti-DUX4 transduced cells suppressed the activation of IFIH1 by lenti-GFP, but did not induce expression of defensin B. Therefore, the inventors concluded that at least part of the suppression of the innate immune response by DUX4-fl is through a secreted factor, which might be DEFB103A/B.

### Summary of Results

The results described in this example demonstrates that DUX4-fl inhibits the innate immune response induced by lenti-viral infection and further that DUX4-fl induces expression of a secreted factor that suppresses the innate immune response.

### Example 6

This Example demonstrates that an agent known to inhibit the histone demethylase LSD1 suppresses DUX4 mRNA levels, and an agent that modifies translation dependent nonsense mediated decay stabilizes DUX4 mRNA levels.

### Methods and Results:

An Agent Previously Shown to Inhibit the Histone Demethylase LSD1 can Suppress DUX4 mRNA Levels

The inventors' previous work demonstrated that DUX4 expression is epigenetically repressed and the inefficient repression of DUX4 that causes FSHD is correlated with decreased repressive histone methylation at lysine nine of histone 3 (H3K9 methylation). The monamine oxidase inhibitor pargyline has been reported to inhibit the activity of the LSD1 histone demethylase that demethylates H3K9. Treatment of FSHD muscle cultures with pargyline suppresses the expression of DUX4 mRNA expression, whereas an MAO inhibitor with a different spectrum of activity does not alter DUX4 mRNA levels.

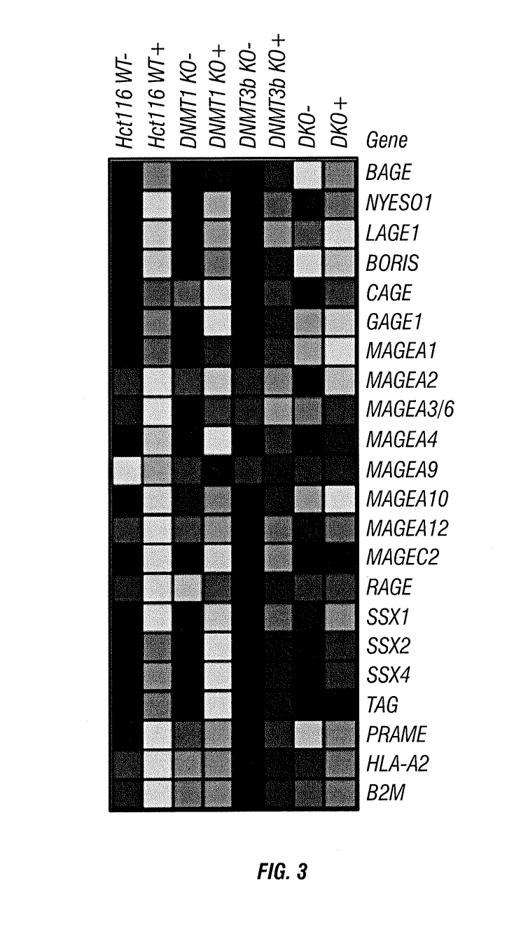

Results showed that pargyline decreases the amount of DUX4 mRNA in FSHD muscle cells (FIG. 4). FSHD muscle cells that express endogenous DUX4-fl mRNA were treated with the MAO inhibitor pargyline that has been reported to inhibit the histone demethylase LSD1, or with another MAO inhibitor tranylcypromine that has a different spectrum of activity. The pargyline decreases the abundance of DUX4-fl mRNA in a dose-dependent manner as measured by quantitative RT-PCR while tranylcypromine had no inhibitory effect as compared to control.

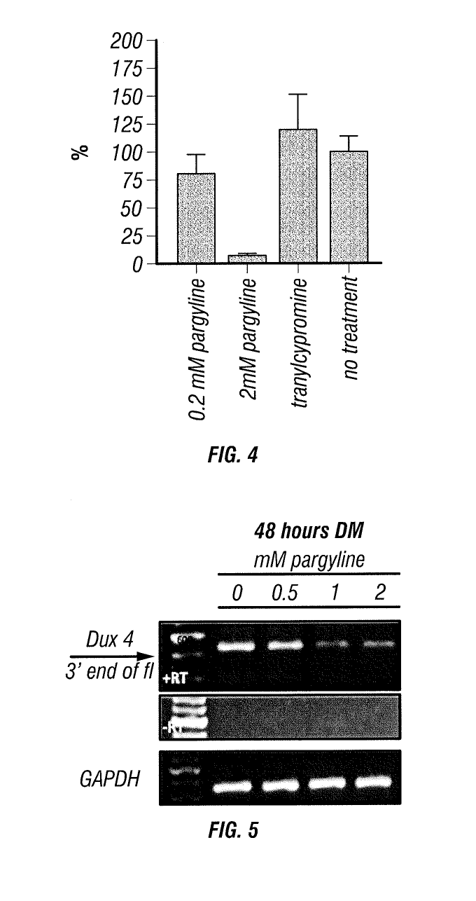

Results showed that pargyline has a dose-dependent inhibition of DUX4 mRNA expression in FSHD muscle cells (FIG. 5). Cultured FSHD muscle cells were differentiated for 48 hours in differentiation medium (DM) with varying amounts of pargyline and the amount of DUX4-fl mRNA was measured by RT-PCR. There was a dose-dependent inhibition of DUX4 expression (top panel). Middle panel is a no RT control and bottom panel is a GAPDH loading control.

The inventors conclude that agents that increase chromatin mediated repression, such as agents that inhibit LSD1 activity, will be useful to suppress DUX4 and are candidate therapeutic agents for FSHD. Such agents are believed to also have application to other diseases, such as myotonic dystrophy or Huntington's disease, where increasing chromatin mediated suppression of the mutant allele would have therapeutic benefit.

An Agent that Modifies Translation Dependent Nonsense Mediated Decay Will Stabilize DUX4 mRNA Levels

The 3-prime untranslated region (UTR) of the DUX4 mRNA has an unusual exon-intron structure. The translational stop codon is in the first exon, whereas the polyadenylation sequence is in the third exon. The separation of the translational stop codon from the polyadenylation site by two exon-intron junctions would be predicted to make this mRNA subject to translation dependent nonsense mediated decay. The inventors can demonstrate that blocking translation with cycloheximide results in the accumulation of the DUX4 mRNA, whereas washing out the cycloheximide and permitting translation of the accumulated DUX4 mRNA (as evidenced by the activation of DUX4 target genes) results in the rapid degradation of the DUX4 mRNA.

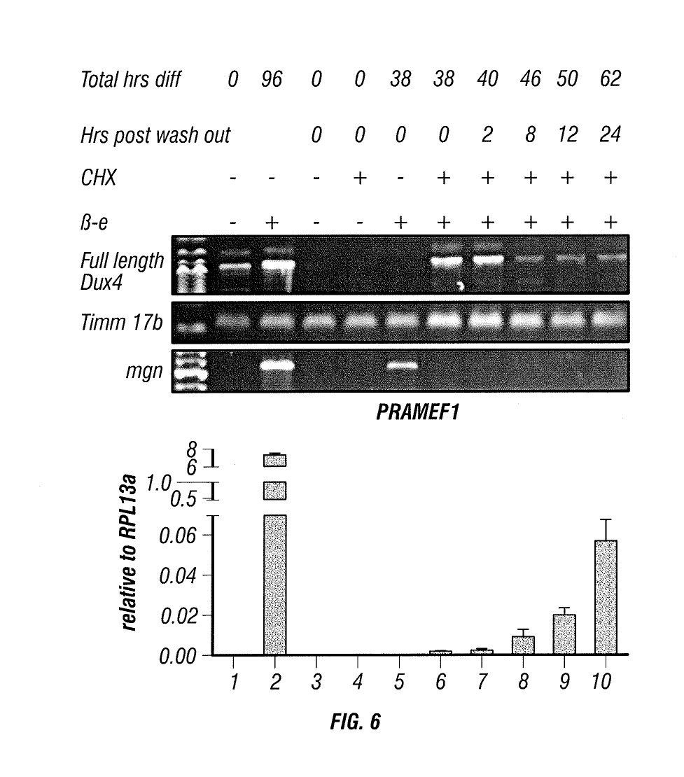

Results showed that the protein synthesis inhibitor cycloheximide (chx) prevents decay of the DUX4 mRNA (FIG. 6). FSHD fibroblasts expressing low amounds of DUX4 mRNA were stably transduced with a beta-estradiol inducible MyoD so that addition of beta-estradiol will convert them to skeletal muscle, which after 96 hours of induction increases the steady-state levels of the DUX4 mRNA and activates expression of the MyoD target Mgn. At 38 hours in differentiation conditions, there is very low abundance of DUX4 mRNA with MyoD induction alone, whereas the addition of chx results in a significant increase in DUX4 mRNA. Washout of the chx results in the rapid loss of the DUX4 mRNA, disappearing between 2 and 8 hrs of washout. The loss of DUX4 mRNA is associated with its translation since the DUX4 target PRAME 1 is induced as the DUX4 mRNA disappears.

Therefore, approaches that block translation dependent nonsense mediated decay can be used to increase DUX4 mRNA and agents that enhance nonsense mediated decay can be used to enhance the degradation of DUX4 mRNA. The latter would be candidate therapies for FSHD.

### Summary of Results:

These results demonstrate that an agent known to inhibit the histone demethylase LSD1 suppresses DUX4 mRNA levels. Therefore, agents that increase chromatin mediated repression, such as agents that inhibit LSD1 activity, will be useful to suppress DUX4 and are candidate therapeutic agents for FSHD. Such agents are believed to also have application to other diseases, such as myotonic dystrophy or Huntington's disease, where increasing chromatin mediated suppression of the mutant allele would have therapeutic benefit.

These results also demonstrate that an agent that modifies translation dependent nonsense mediated decay stabilizes DUX4 mRNA levels. Therefore, approaches that block translation dependent nonsense mediated decay can be used to increase DUX4 mRNA and agents that enhance nonsense mediated decay can be used to enhance the degradation of DUX4 mRNA, which provides a candidate therapy for FSHD.

It is further noted, as described above in Example 2, DUX4-s can bind the same sequences as DUX4-fl but does not activate transcription of the same genes. Therefore, DUX4-s functions as an inhibitor of DUX4-fl and can also be used as an inhibitor for FSHD.

### Example 7

This Example demonstrates that activation of germline genes in FSHD muscle cells is directly due to the leaky expression of DUX4.

### Rationale:

As described in Example 3, the inventors have demonstrated that DUX4 regulates the expression of many genes expressed almost exclusively in the germline, some of which have known functions in meiosis and gametogenesis. As further described in Example 3, the inventors have also found expression of the DUX4-related genes DUXA and DUX1 in the testis, indicating the likelihood of multiple redundant factors. In order to confirm there is a functional link between DUX4 induction and molecular changes in FSHD muscle, the following siRNA knock-down experiment was carried out which demonstrates that the activation of germline genes in FSHD muscle cells is directly due to the leaky expression of DUX4.

### Methods:

siRNA Knockdown of DUX4

siRNAs (Dharmacon) targeted to the mature mRNA of DUX4 and a control siRNA against luciferase were used as follows:

(SEQ ID NO: 179)
Control siRNA 5′-r(CUUACGCUGAGUACUUCGA)d(TT)-3′
(SEQ ID NO: 180)
DUX4 siRNA-1 5′-r(GAGCCUGCUUUGAGCGGAA)d(TT)-3′
(SEQ ID NO: 181)
DUX4 siRNA-2 5′-r(GCGCAACCUCUCCUAGAAA)d(TT)-3′
(SEQ ID NO: 182)
DUX4 siRNA-3 5′-r(CAAACCUGGAUUAGAGUUA)d(TT)-3′
(SEQ ID NO: 183)
DUX4 siRNA-4 5′-r(GAUGAUUAGUUCAGAGAUA)d(TT)-3′

Cultured FSHD myoblasts were transfected in 35 mm dishes with 30 pmol of siRNA using RNAiMAX (Invitrogen) according to manufacturer's recommendations. Following overnight incubation with siRNA complexes, cells were washed and allowed to recover for 12-24 hours in fresh growth media (F10, 20% FBS, 1 μM dexamethasone, 0.01 μg/ml FGF). When confluent, cultures were changed to differentiation media (F10, 1% horse serum, 10 μg/ml each insulin and transferrin,) for 48 hours. RNA was isolated using RNeasy Miniprep Kit (Qiagen), RT and PCR protocols were performed as described in Snider et al. (2010) using primers 1A (SEQ ID NO:164) and 183 (SEQ ID NO:163).

### DUX4-fl PCR

Nested DUX4-fl3′ PCR on primary myoblast and muscle biopsies were performed as described in Snider et al., PloS Genet 6, e1001181 (2010). Primers used were 182 forward (SEQ ID NO: 162) and 183 reverse (SEQ ID NO:163) nested with 1A forward (SEQ ID NO:164) and 184 reverse (SEQ ID NO:165).

### Dominant Negative Inhibition of DUX4-fl

Cultured FSHD myoblasts were grown to confluence and switched to differentiation media as described above. Simultaneously, cells were transduced by lentivirus carrying DUX4-s or GFP along with 8 μg/mL polybrene. Cells were washed and changed to plain differentiation media after 24 hours. Cells were harvested for RNA after 48 hours of differentiation. Untransduced cells were used to assess baseline expression of DUX4-fl target genes.

### Results:

The siRNA sequences that decreased the DUX4-fl mRNA also resulted in decreased expression of the DUX4 target genes, confirming that endogenous DUX4 drives the expression of these genes in FSHD muscle cells.

Results showed that, in a RT-PCR assay, siRNA knockdown of endogenous DUX4-fl in cultured FSHD muscle cells, done in triplicate with Timm17b as an internal standard. Negative control siRNA is against unrelated luciferase gene. Further results showed the results of qPCR analysis of DUX4-fl target genes in the presence of siRNA to endogenous DUX4-fl, relative to the control treated samples, demonstrating that the levels of DUX4-fl target genes were also reduced when endogenous DUX4-fl was knocked down. Error bars represent standard deviation of triplicates, *P<0.05, **P<0.01 between DUX4 siRNA and control siRNA treated cells.

Results of real-time RT-PCR quantitation of three target genes, PRAMEF1 (A), RFPL2 (B) and MBD3L2 (C) in FSHD cultured muscle cells transduced with lenti-GFP or lenti-DUX4-s or untransduced controls were obtained. The abundance of targets was calculated relative to internal standard RPL13a and then set as percentages relative to the untransduced condition. Values represent mean+/−SEM from three independent experiments. DUX4-s blocks expression of these three DUX4-fl target genes in FSHD muscle cells. These results are consistent with the results described in Example 3, demonstrating that expression of the dominant negative DUX4-s also inhibited the endogenous expression of the target genes. These results confirm that the activation of germline genes in FSHD muscle cells is due to the leaky expression of DUX4 in FSHD muscle cells.

Therefore, the results shown in this example demonstrate that agents that inhibit the activity of DUX4, either by eliminating its expression in the muscle cells, as done in vitro with an siRNA, or by introducing a dominant negative agent, such as the DUX4-s splice form are expected to be useful as therapeutic agents for treating and/or preventing FSHD, or symptoms related to FSHD.

### Example 8

This Example demonstrates that DUX4-induced DEFB103 inhibits the innate immune response and muscle differentiation.

### Rationale:

As described in Example 5, the inventors have determined that genes enriched in the innate immunity pathway were expressed at lower levels in myoblasts transduced with lenti-DUX4 compared to the lenti-GFP or lenti-DUX4-s. When compared to non-transduced cells, it was evident that about 350 genes, as shown in TABLE 11 and as shown in updated TABLE 13 included below, most of which were in the innate immunity pathway, were unchanged in the lenti-DUX4-fl transduced myoblasts but increased in cells transduced with either control lenti-GFP or lenti-DUX4-s. Therefore, lentiviral induction of the innate immune response in human muscle cells appear to be inhibited by DUX4-fl.

In this Example, experiments were carried out to further determine the effects of DUX4-fl induced expression on the innate immunity pathway and the myogenesis pathway.

### Methods:

Real time RT-PCR quantitation of innate immune responsive genes and genes involved in muscle differentiation was carried out on lenti-GFP infected cells, lenti-DUX4-fl infected cells and uninfected control cells, as described below.

### Beta-Defensin 3 and Innate Immune Response

Cultured control human myoblasts were grown to 80% confluence and infected with equivalent titers of lenti-GFP, lenti-DUX4-s and lenti-DUX4-fl in growth media supplemented with 8 μg/mL polybrene. Expression of innate immune responsive genes including IFIH1 (MDA5), ISG20 and DEFB103 were assessed by real-time qPCR as previously described at 24 hours (primer sequences below). For conditioned media, cells were infected with lenti-DUX4-fl for 12 hours, thoroughly washed 3 times with PBS and switched to fresh growth media to condition for 12 hours. Control conditioned media was produced from cells not infected with any lentivirus. Myoblasts were infected with lenti-GFP in either control conditioned media, lenti-DUX4-fl conditioned media or regular growth media supplemented with 1 μM human β-defensin 3 (Peptides International, Louisville, Ky.). Expression of innate immune responsive genes were examined after 24 hours.

| Gene | Forward primer | Reverse primer |
|---|---|---|
| name | sequence | sequence |
| IFIH1 | CTAGCCTGTTCTGGGGAAGA | AGTCGGCACACTTCTTTTGC |
| | (SEQ ID NO: 184) | (SEQ ID NO: 185 |
| ISG20 | GAGCGCCTCCTACACAAGAG | CGGATTCTCTGGGAGATTTG |
| | (SEQ ID NO: 186) | (SEQ ID NO: 187) |
| DEFB103 | TGTTTGCTTTGCTCTTCCTG | CGCCTCTGACTCTGCAATAA |
| | (SEQ ID NO: 188) | (SEQ ID NO: 189) |

### Beta-Defensin 3 and Muscle Differentiation

Cultured control myoblasts were grown at 50% confluence and treated with 1 μM human β-defensin 3. Equivalent volume of vehicle (water) was added to myoblasts for the control condition. Quadruplicate samples of control- and DEFB103-treated myoblasts were assessed for global expression changes on HumanHT-12 v4 Expression BeadChip Whole Genome arrays and analyzed as described in main methods. Differential expression of myostatin (MSTN) was confirmed by real-time qPCR.

| Gene | Forward primer | Reverse primer |
|---|---|---|
| name | sequence | sequence |
| MSTN | CTGTAACCTTCCCAGGACCA | TCCCTTCTGGATCTTTTTGG |
| | (SEQ ID NO: 190) | (SEQ ID NO: 191) |

Cultured control myoblasts were grown to confluence and switched to differentiation media (as described in Snider et al., 2010). 24 hours later, media was refreshed and either supplemented with 1 μM human β-defensin 3 or equivalent volume of water. Media was refreshed again at 48 hours. Cells were differentiated for a total of 72 hours. Quadruplicate samples were analyzed by expression microarrays as described above. Differential expression of various markers of muscle differentiation were confirmed by real-time qPCR using the primers shown below (see above for CKM and MYH2 primer sequences).

| Gene | Forward primer | Reverse primer |
|---|---|---|
| name | sequence | sequence |
| ACTA1 | GTACCCTGGGATCGCTGAC | CCGATCCACACCGAGTATTT |
| | (SEQ ID NO: 192) | (SEQ ID NO: 193) |
| CASQ2 | AGATTGGGGTGGTGAATGTC | TCCTCAATCCAGTCCTCCAG |
| | (SEQ ID NO: 194) | (SEQ ID NO: 195) |
| TNNT3 | CAAGTTCGAGTTTGGGGAGA | AGCCTTCTTGCTGTGCTTCT |
| | (SEQ ID NO: 196) | (SEQ ID NO: 197) |
| MYF6 | GCCAAGTGTTTCCGATCATT | CACGATGGAAGAAAGGCATC |
| | (SEQ ID NO: 198) | (SEQ ID NO: 199) |
| DESMIN | GATCAATCTCCCCATCCAGA | TGGCAGAGGGTCTCTGTCTT |
| | (SEQ ID NO: 200) | (SEQ ID NO: 201) |

### Beta-Defensin 3 (DEFB103) and Myotube Formation in Primary Muscle

Cultured primary muscle cells were cultured in differentiation medium for 72 hours in the presence or absence of 0.5 uM Human β-defensin 3. The cells were then immunostained for myosin heavy chain and nuclei, showing that DEFB103 inhibits muscle cell fusion and expression of myosin heavy chain, as compared to the muscle cell cultures that do not contain DEFB103.

As shown in TABLE 13 below, the inventors have determined that genes enriched in the innate immunity pathway were unchanged in the lenti-DUX4-fl transduced myoblasts but increased in cells transduced with either control lenti-GFP or lenti-DUX4-s. Therefore, lentiviral induction of the innate immune response in human muscle cells appears to be inhibited by DUX4-fl.

TABLE 13
Genes induced by lenti-GFP and lenti-DUX4-s but poorly induced by lenti-DUX4-fl

| | GFP v. | | Short_v. GFP | Full v._NoLenti | Short_vs. |
|---|---|---|---|---|---|
| Gene symbol | NoLenti (Fc) | Full_v. GFP (Fc) | (Fc) | (Fc) | NoLenti (Fc) |
| ABCA1 | 2.291820276 | −1.188123428 | −0.317144691 | 1.103696848 | 1.974675585 |
| ABI3BP | 1.285349969 | −1.242569141 | −0.399190433 | 0.042780827 | 0.886159535 |
| ACSM5 | 1.171658705 | −1.210123921 | 0.049346191 | −0.038465216 | 1.221004895 |
| ADAR | 1.360446453 | −2.320782749 | 0.316391226 | −0.960336295 | 1.676837679 |
| ADAR | 1.486951815 | −2.180439697 | 0.356871011 | −0.693487882 | 1.843822826 |
| ADCK3 | 1.64121942 | −1.422215254 | −0.173131237 | 0.219004166 | 1.468088183 |
| AGAP1 | 1.390382407 | −1.271915967 | 0.522414096 | 0.11846644 | 1.912796503 |
| AGRN | 2.630293701 | −2.531537554 | 0.379640013 | 0.098756147 | 3.009933713 |
| AK3 | 1.073902409 | −1.251916684 | −0.421459526 | −0.178014275 | 0.652442883 |
| ALDH3A2 | 1.125411852 | −1.242892954 | −0.194265137 | −0.117481102 | 0.931146715 |
| ALOX15B | 1.084246479 | −1.214364727 | 0.715187163 | −0.130118248 | 1.799433642 |
| ANGPT1 | 1.027654313 | −1.084975107 | 0.217446704 | −0.057320794 | 1.245101017 |
| ANKRA2 | 1.035862621 | −1.17479048 | 0.327844812 | −0.138927859 | 1.363707433 |
| ANKRA2 | 1.143001219 | −1.343557009 | 0.246235239 | −0.20055579 | 1.389236458 |
| ANPEP | 1.4045147 | −2.09404032 | 0.355618263 | −0.689525619 | 1.760132964 |
| APCDD1 | 1.816660994 | −1.702791884 | −0.409640643 | 0.11386911 | 1.407020351 |
| APOBEC3G | 1.356603191 | −1.743348451 | 1.362079936 | −0.38674526 | 2.718683128 |
| ATL3 | 1.172765182 | −1.43324744 | 1.336632204 | −0.260482258 | 2.509397386 |
| BATF2 | 1.051676687 | −1.066958424 | 1.271438373 | −0.015281737 | 2.32311506 |
| BCL3 | 1.076324149 | −1.198088718 | −0.043769492 | −0.121764569 | 1.032554657 |
| BCL6 | 1.239304605 | −1.250683236 | 0.359886105 | −0.011378631 | 1.59919071 |
| BIRC3 | 1.783961421 | −2.14067143 | 1.056849919 | −0.356710009 | 2.84081134 |
| BST2 | 4.693875549 | −4.110609557 | 0.211256373 | 0.583265992 | 4.905131922 |
| BTG2 | 1.24201129 | −1.524684273 | −0.121180138 | −0.282672983 | 1.120831152 |
| BTN3A2 | 1.396364956 | −1.575530186 | 0.323881527 | −0.17916523 | 1.720246483 |
| BTN3A3 | 1.342617431 | −1.39213812 | 0.373702947 | −0.049520689 | 1.716320378 |
| C10orf10 | 2.191627592 | −2.507058994 | 0.142569335 | −0.315431402 | 2.334196927 |
| C14orf159 | 1.258024351 | −1.957895816 | 0.509946226 | −0.699871465 | 1.767970578 |
| C18orf56 | 1.451934043 | −1.513673871 | 0.209056629 | −0.061739828 | 1.660990672 |
| C19orf66 | 1.714465108 | −2.178084015 | 0.603885269 | −0.463618907 | 2.318350377 |
| C1QTNF1 | 3.514164258 | −3.63617124 | 0.762580688 | −0.122006982 | 4.276744946 |
| C1R | 3.431740878 | −2.764990793 | 0.489024106 | 0.666750085 | 3.920764984 |
| C1R | 4.097498539 | −2.991070279 | 0.476766971 | 1.10642826 | 4.574265511 |
| C1RL | 1.210460055 | −1.453877289 | −0.149539045 | −0.243417235 | 1.060921009 |
| C1S | 3.50755039 | −2.743766253 | 0.538006932 | 0.763784136 | 4.045557322 |
| C4orf34 | 1.457645505 | −1.036411533 | 0.604138258 | 0.421233972 | 2.061783763 |
| C6orf138 | 1.28233824 | −1.176619381 | 0.946386736 | 0.105718859 | 2.228724976 |
| C9orf169 | 1.550194985 | −2.339507952 | −0.049415733 | −0.789312967 | 1.500779252 |
| CA12 | 1.505275285 | −2.358845458 | 0.93380504 | −0.853570172 | 2.439080325 |
| CA9 | 1.383310651 | −1.314316803 | 0.489105112 | 0.068993848 | 1.872415763 |
| CABYR | 1.134071595 | −1.591154258 | −0.041503054 | −0.457082663 | 1.092568541 |
| CCL2 | 2.390739575 | −2.377452771 | 0.425667754 | 0.013286804 | 2.816407329 |
| CCL5 | 2.46842308 | −2.245603872 | 1.863386716 | 0.222819207 | 4.331809795 |
| CCL5 | 2.792121924 | −3.05224536 | 1.431316683 | −0.260123436 | 4.223438607 |
| CCND2 | 1.345983368 | −1.046404064 | −0.528969223 | 0.299579305 | 0.817014146 |
| CCND2 | 1.211684627 | −1.010372009 | −0.539017483 | 0.201312618 | 0.672667144 |
| CD68 | 2.369427793 | −1.085666614 | 1.175094172 | 1.283761179 | 3.544521965 |
| CDK18 | 1.479105183 | −1.409658202 | 0.55520404 | 0.069446981 | 2.034309223 |
| CDKN1A | 1.577318436 | −2.005503982 | 0.150353898 | −0.428185547 | 1.727672334 |
| CEBPB | 1.316137124 | −2.448210379 | 0.278125966 | −1.132073255 | 1.59426309 |
| CEBPD | 2.027652082 | −1.536750261 | 0.1644903 | 0.490901821 | 2.192142382 |
| CES2 | 1.211182709 | −2.015465315 | 0.060436142 | −0.804282606 | 1.271618851 |
| CES2 | 1.462241013 | −1.322576234 | 0.297278159 | 0.139664779 | 1.759519172 |
| CFB | 5.389687275 | −5.32123731 | 0.655123952 | 0.068449966 | 6.044811228 |
| CFD | 1.460205956 | −1.09433816 | 0.241365677 | 0.365867797 | 1.701571633 |
| CFLAR | 1.173687322 | −1.870286111 | 0.531638717 | −0.696598789 | 1.70532604 |
| CHI3L2 | 2.883508876 | −2.453932636 | 0.25288949 | 0.429576241 | 3.136398366 |
| CIDEC | 1.295153579 | −1.12502825 | −0.027982338 | 0.17012533 | 1.267171242 |
| CLDN15 | 1.762738312 | −2.317560734 | −0.503444118 | −0.554822422 | 1.259294194 |
| CLMP | 1.100423145 | −1.139438434 | 0.02079778 | −0.03901529 | 1.121220924 |
| CMBL | 1.779468214 | −1.956455153 | 0.067785064 | −0.176986938 | 1.847253278 |
| COL7A1 | 1.672860855 | −2.086299714 | −0.187476705 | −0.413438859 | 1.48538415 |
| COPS8 | 1.084649605 | −1.235003851 | 0.143856969 | −0.150354246 | 1.228506574 |
| CORO6 | 1.154149699 | −1.09307255 | −0.292855728 | 0.061077149 | 0.861293971 |
| CSF3 | 1.306379572 | −1.379014306 | 0.751058681 | −0.072634734 | 2.057438253 |
| CSF3 | 1.170619642 | −1.285172578 | 0.512509223 | −0.114552936 | 1.683128865 |
| CST3 | 1.352916208 | −1.114340315 | 0.173115486 | 0.238575894 | 1.526031695 |
| CXCL1 | 3.161742919 | −3.2056022 | 0.199746505 | −0.043859282 | 3.361489423 |
| CXCL2 | 1.088531774 | −1.12427504 | 0.000297431 | −0.035743266 | 1.088829206 |
| CXCL5 | 1.796732217 | −2.163410121 | 0.802217283 | −0.366677904 | 2.5989495 |
| CXCL5 | 2.552762132 | −2.7972733 | 0.752156307 | −0.244511169 | 3.304918439 |
| CXCL6 | 1.612320374 | −1.711616386 | 0.16209417 | −0.099296012 | 1.774414544 |
| CXCL6 | 2.889344416 | −2.764659712 | 0.355794239 | 0.124684703 | 3.245138655 |
| CYBASC3 | 1.566585338 | −1.92049503 | −0.418331779 | −0.353909692 | 1.148253559 |
| CYFIP2 | 1.388031804 | −2.080860129 | 0.269013837 | −0.692828325 | 1.657045641 |
| CYGB | 1.242739213 | −1.222078642 | 0.144472485 | 0.020660571 | 1.387211698 |
| CYP26B1 | 1.557009643 | −1.35288291 | 0.402951144 | 0.204126733 | 1.959960788 |
| CYP27A1 | 1.603224607 | −1.558991917 | −0.059213982 | 0.04423269 | 1.544010625 |
| DCN | 1.780361473 | −1.557663269 | 0.260180776 | 0.222698204 | 2.040542249 |
| DCN | 1.255744448 | −1.364162449 | 0.516581942 | −0.108418 | 1.77232639 |
| DCN | 2.535469628 | −2.115329344 | 0.20630244 | 0.420140284 | 2.741772068 |
| DDB2 | 1.065126371 | −1.227252657 | 0.098724876 | −0.162126285 | 1.163851248 |
| DDR2 | 2.23329097 | −2.184044969 | 0.628165913 | 0.049246001 | 2.861456882 |
| DDX58 | 2.122646261 | −2.302586875 | 0.020336733 | −0.179940614 | 2.142982994 |
| DDX60 | 1.857459371 | −2.114354393 | 0.440034021 | −0.256895022 | 2.297493392 |
| DDX60L | 1.165403911 | −1.453009147 | 0.631525336 | −0.287605236 | 1.796929248 |
| DGKA | 1.146131144 | −1.731341707 | 0.097641823 | −0.585210562 | 1.243772967 |
| DGKA | 1.538412659 | −2.05990981 | 0.223417083 | −0.521497151 | 1.761829742 |
| DHX58 | 2.354550923 | −2.524320066 | 0.681738729 | −0.169769142 | 3.036289652 |
| DKK1 | 2.058791481 | −2.299442613 | 0.466215843 | −0.240651132 | 2.525007325 |
| DRAM1 | 1.168624381 | −1.055889591 | −0.358650503 | 0.112734791 | 0.809973878 |
| DUSP10 | 1.052825287 | −1.721052291 | −0.082362184 | −0.668227004 | 0.970463102 |
| DUSP19 | 1.515692701 | −2.35548253 | 0.759827653 | −0.839789829 | 2.275520354 |
| EGFR | 1.23501357 | −1.29432245 | 1.188755373 | −0.05930888 | 2.423768943 |
| EIF2AK2 | 2.716902652 | −2.268220749 | 0.552783876 | 0.448681903 | 3.269686528 |
| EPSTI1 | 4.363652437 | −4.026958202 | 0.683321824 | 0.336694234 | 5.046974261 |
| ERAP2 | 1.617866844 | −1.564993767 | 0.765635296 | 0.052873078 | 2.38350214 |
| FAM160B1 | 1.895947832 | −1.553397895 | 1.542319975 | 0.342549937 | 3.438267807 |
| FAM198B | 1.372529451 | −1.749916317 | −0.511628689 | −0.377386866 | 0.860900762 |
| FAM198B | 1.564969517 | −1.666998002 | −0.288322282 | −0.102028485 | 1.276647235 |
| FBXO32 | 2.567095797 | −1.157656064 | 0.762440333 | 1.409439733 | 3.32953613 |
| FBXO32 | 2.993395576 | −1.797189568 | 0.768145422 | 1.196206008 | 3.761540997 |
| FILIP1L | 1.21285947 | −1.72307622 | 0.063505825 | −0.51021675 | 1.276365295 |
| FKBP5 | 1.059782793 | −1.768400334 | −0.105221181 | −0.708617541 | 0.954561612 |
| FOXQ1 | 1.487355619 | −1.213797134 | 0.586270597 | 0.273558485 | 2.073626216 |
| FRMD3 | 1.264347851 | −1.008896541 | −0.268549855 | 0.25545131 | 0.995797996 |
| FST | 1.879306676 | −1.797658416 | 0.915428493 | 0.08164826 | 2.79473517 |
| FTH1 | 1.16763403 | −2.003091159 | −0.411200627 | −0.835457129 | 0.756433403 |
| FTH1P3 | 1.578547897 | −2.546551924 | 0.458681522 | −0.968004027 | 2.037229419 |
| FUCA1 | 1.553243173 | −1.53000218 | 0.284588564 | 0.023240994 | 1.837831738 |
| GALNTL2 | 1.627249768 | −1.600577362 | 0.951168181 | 0.026672406 | 2.578417949 |
| GAS1 | 1.248292919 | −1.730183911 | 0.006982613 | −0.481890992 | 1.255275533 |
| GAS1 | 1.307924743 | −1.545046265 | 0.089615034 | −0.237121521 | 1.397539777 |
| GBP1 | 1.663035588 | −2.204470258 | 0.995466771 | −0.54143467 | 2.658502359 |
| GBP1 | 1.539177456 | −1.911140496 | 1.032949835 | −0.37196304 | 2.572127291 |
| GBP2 | 2.194934144 | −2.389860644 | 0.677298854 | −0.1949265 | 2.872232998 |
| GBP4 | 1.335924885 | −1.254995665 | 2.240198911 | 0.08092922 | 3.576123797 |
| GDF15 | 1.771872719 | −2.053594964 | 0.915096518 | −0.281722245 | 2.686969237 |
| GFPT2 | 1.729215691 | −1.590429831 | 0.049458918 | 0.13878586 | 1.778674609 |
| GRINA | 1.773238707 | −1.404244628 | 0.466739167 | 0.368994079 | 2.239977874 |
| GRINA | 1.161955946 | −1.198733642 | 0.421477903 | −0.036777696 | 1.583433849 |
| H1F0 | 1.210846088 | −2.357976468 | 0.498399795 | −1.14713038 | 1.709245883 |
| HCG4 | 1.247551934 | −1.625383459 | 0.675501339 | −0.377831525 | 1.923053273 |
| HECW2 | 1.217292839 | −1.048324787 | 0.055161117 | 0.168968052 | 1.272453956 |
| HERC5 | 5.367300538 | −3.156622521 | 1.346816689 | 2.210678018 | 6.714117227 |
| HERC6 | 3.892685006 | −3.57126233 | 0.518426791 | 0.321422676 | 4.411111797 |
| HIPK2 | 1.273540096 | −1.156200641 | 0.35601076 | 0.117339455 | 1.629550856 |
| HLA-A | 1.224282572 | −1.070849545 | 0.574982093 | 0.153433026 | 1.799264664 |
| HLA-B | 3.448509688 | −2.752055852 | 0.333358529 | 0.696453836 | 3.781868217 |
| HLA-C | 2.043797634 | −1.72804812 | 0.602243193 | 0.315749513 | 2.646040827 |
| HLA-E | 2.489318232 | −1.122898369 | 0.809237995 | 1.366419864 | 3.298556227 |
| HLA-F | 1.873709807 | −1.735091355 | 0.913162292 | 0.138618452 | 2.786872099 |
| HLA-F | 2.704239531 | −2.113067715 | 0.61482352 | 0.591171816 | 3.319063051 |
| HLA-G | 1.084389593 | −1.205707251 | 0.109199209 | −0.121317658 | 1.193588802 |
| HLA-H | 1.297130552 | −1.669181838 | 0.321169353 | −0.372051286 | 1.618299905 |
| HLA-H | 2.553573965 | −2.193531507 | 0.353939424 | 0.360042458 | 2.90751339 |
| HOXC13 | 1.000534735 | −1.088749573 | 0.444564824 | −0.088214838 | 1.445099559 |
| IFI16 | 1.841068311 | −1.984982422 | 0.679092735 | −0.143914111 | 2.520161046 |
| IFI27 | 7.303388227 | −6.379335039 | 0.300265465 | 0.924053188 | 7.603653692 |
| IFI35 | 3.410532516 | −3.729262173 | 0.92164533 | −0.318729656 | 4.332177846 |
| IFI44 | 3.287546389 | −2.389816265 | 0.30094931 | 0.897730124 | 3.588495698 |
| IFI44L | 5.753370016 | −5.011530642 | 0.464189819 | 0.741839374 | 6.217559834 |
| IFI6 | 5.085469725 | −4.262101551 | −0.040327545 | 0.823368174 | 5.04514218 |
| IFIH1 (MDA5) | 3.693641619 | −3.44578554 | 1.642722308 | 0.247856079 | 5.336363928 |
| IFIT1 | 4.787387527 | −4.356740033 | 0.716020264 | 0.430647493 | 5.503407791 |
| IFIT2 | 4.365674354 | −4.164948286 | 2.078380504 | 0.200726068 | 6.444054859 |
| IFIT3 | 3.681888585 | −3.640974725 | 1.807701431 | 0.040913859 | 5.489590016 |
| IFIT3 | 5.228105553 | −4.765773435 | 1.104511852 | 0.462332118 | 6.332617405 |
| IFIT3 | 3.365518868 | −3.151248319 | 0.534775341 | 0.214270549 | 3.900294209 |
| IFIT5 | 1.071262503 | −1.42655834 | −0.257503173 | −0.355295837 | 0.81375933 |
| IFITM1 | 6.728483235 | −5.998523582 | 0.346633408 | 0.729959653 | 7.075116644 |
| IFITM2 | 1.287202103 | −1.750085601 | 0.0071809 | −0.462883498 | 1.294383003 |
| IFITM3 | 1.982789092 | −1.995925104 | −0.140675202 | −0.013136012 | 1.84211389 |
| IGDCC4 | 1.215267655 | −1.594375685 | −0.624900224 | −0.37910803 | 0.59036743 |
| IGFBP4 | 2.222133478 | −1.926807782 | 0.418741062 | 0.295325696 | 2.64087454 |
| IGFBP5 | 1.215794572 | −1.28188317 | 0.168705129 | −0.066088598 | 1.384499701 |
| IGFBP5 | 1.539901788 | −1.310867444 | 0.281298714 | 0.229034345 | 1.821200502 |
| IL18BP | 2.005806379 | −1.15855931 | 1.366694726 | 0.847247069 | 3.372501105 |
| IL1R1 | 1.321158201 | −1.23340973 | 0.287796205 | 0.087748472 | 1.608954406 |
| IL32 | 2.025999493 | −2.208173565 | 0.154358881 | −0.182174073 | 2.180358374 |
| IL7R | 1.506258978 | −1.99732977 | 0.548115296 | −0.491070792 | 2.054374274 |
| IL8 | 3.516952055 | −3.648099533 | 1.170200764 | −0.131147477 | 4.687152819 |
| IL8 | 5.331346339 | −5.451111992 | 0.930533775 | −0.119765653 | 6.261880114 |
| IRAK3 | 1.353708064 | −1.342339713 | 1.020300119 | 0.01136835 | 2.374008182 |
| IRF7 | 2.864351588 | −1.844318439 | 1.125390895 | 1.020033148 | 3.989742483 |
| IRF7 | 2.755432373 | −2.160487542 | 0.95548081 | 0.594944831 | 3.710913184 |
| IRF9 | 2.42820399 | −2.232627679 | 0.295571895 | 0.195576311 | 2.723775884 |
| ISG15 | 5.06042249 | −4.31794577 | 0.141645299 | 0.74247672 | 5.202067789 |
| ISG20 | 2.30394629 | −2.679339661 | 2.329592064 | −0.375393371 | 4.633538353 |
| ITPRIP | 1.1013701 | −1.710399371 | 0.554083786 | −0.60902927 | 1.655453886 |
| KIAA0247 | 2.303673186 | −2.013448857 | 0.358981191 | 0.290224329 | 2.662654377 |
| KLF9 | 1.023238226 | −1.245660536 | −0.052040135 | −0.22242231 | 0.971198091 |
| KRT17 | 1.380656082 | −1.409415072 | 0.431567127 | −0.02875899 | 1.81222321 |
| KYNU | 1.119459921 | −1.093624095 | 1.337790233 | 0.025835826 | 2.457250155 |
| LAP3 | 1.749851576 | −1.537094669 | 1.409981585 | 0.212756907 | 3.159833161 |
| LGALS3BP | 2.681022143 | −2.112677157 | 0.317090182 | 0.568344986 | 2.998112325 |
| LNPEP | 1.763130709 | −1.279670486 | 2.052682291 | 0.483460223 | 3.815813 |
| LTBR | 1.168565135 | −1.14953269 | 0.400952848 | 0.019032446 | 1.569517983 |
| LUM | 1.151065803 | −1.112365335 | 0.386054713 | 0.038700469 | 1.537120516 |
| LY6E | 3.685056133 | −3.242347639 | 0.257332792 | 0.442708494 | 3.942388925 |
| MAMDC2 | 1.733193514 | −1.841626819 | −0.027430153 | −0.108433305 | 1.705763361 |
| MAOA | 1.417318353 | −1.497995721 | −0.34587423 | −0.080677369 | 1.071444123 |
| MLKL | 1.566078589 | −2.71738901 | 1.443379629 | −1.151310421 | 3.009458218 |
| MME | 1.086772749 | −1.03372451 | −0.182908298 | 0.053048238 | 0.903864451 |
| MMP3 | 1.238760776 | −1.286719097 | 0.258499598 | −0.047958321 | 1.497260374 |
| MMP7 | 1.284665197 | −1.062720937 | 0.191250354 | 0.22194426 | 1.475915551 |
| MOCOS | 1.129068005 | −1.661858991 | 0.417460239 | −0.532790986 | 1.546528244 |
| MR1 | 1.191026064 | −1.17841186 | 0.658029657 | 0.012614204 | 1.849055721 |
| MSI2 | 1.97398629 | −1.695161066 | 1.317806308 | 0.278825224 | 3.291792598 |
| MT1F | 1.224979554 | −1.148897962 | −0.529782109 | 0.076081593 | 0.695197445 |
| MT1G | 1.293754959 | −1.240605881 | 0.255687444 | 0.053149078 | 1.549442403 |
| MT1M | 2.698286944 | −2.872732992 | 1.198565544 | −0.174446049 | 3.896852488 |
| MT1X | 1.981216 | −2.450995611 | 0.310879734 | −0.469779611 | 2.292095734 |
| MTSS1 | 1.331426508 | −1.069633876 | −0.39218049 | 0.261792632 | 0.939246018 |
| MTSS1 | 2.167447769 | −1.354900669 | −0.443277442 | 0.8125471 | 1.724170327 |
| MUC1 | 1.431503927 | −1.575675409 | 0.338131504 | −0.144171482 | 1.769635431 |
| MUC1 | 1.203764531 | −2.101829876 | 0.322803033 | −0.898065345 | 1.526567564 |
| MUSK | 1.324782191 | −1.183254592 | 0.586577408 | 0.141527599 | 1.911359599 |
| MX1 | 7.205558299 | −5.623173414 | 0.288267363 | 1.582384885 | 7.493825662 |
| MX2 | 3.852926817 | −3.778486728 | 0.945818472 | 0.074440089 | 4.798745289 |
| MYBPHL | 1.409152912 | −1.384607359 | −0.499125714 | 0.024545554 | 0.910027198 |
| NACC2 | 1.184709948 | −1.618000195 | 0.95658574 | −0.433290247 | 2.141295688 |
| NDRG1 | 1.630384529 | −2.292807858 | 0.654936282 | −0.66242333 | 2.285320811 |
| NDUFA4L2 | 1.149943565 | −1.122951133 | 0.177144155 | 0.026992433 | 1.32708772 |
| NFE2L2 | 1.280834247 | −1.085476789 | 0.32469271 | 0.195357458 | 1.605526957 |
| NFIL3 | 1.843454665 | −1.768559534 | 0.321122226 | 0.074895131 | 2.16457689 |
| NFKBIA | 2.185485916 | −3.012569006 | 0.269962031 | −0.82708309 | 2.455447947 |
| NFKBIZ | 1.845887352 | −1.544280602 | −0.266011822 | 0.30160675 | 1.57987553 |
| NRCAM | 1.109271808 | −1.109495143 | −0.100930752 | −0.000223335 | 1.008341055 |
| NTPCR | 1.001528266 | −1.091034449 | 0.353533227 | −0.089506183 | 1.355061493 |
| OAS1 | 4.380499828 | −4.254302153 | 1.43883568 | 0.126197675 | 5.819335508 |
| OAS1 | 3.600570008 | −3.699570683 | 1.298090435 | −0.099000675 | 4.898660443 |
| OAS1 | 3.760626423 | −3.807467129 | 1.290300518 | −0.046840706 | 5.050926941 |
| OAS2 | 2.378995669 | −2.376411131 | 0.439887914 | 0.002584538 | 2.818883583 |
| OAS2 | 1.558066832 | −1.597321635 | 0.992880408 | −0.039254803 | 2.55094724 |
| OAS2 | 5.94172586 | −5.520599629 | 0.386635011 | 0.421126231 | 6.32836087 |
| OAS3 | 3.731806761 | −3.817466129 | 0.853256816 | −0.085659368 | 4.585063577 |
| OASL | 1.249672439 | −1.117056273 | 1.634341212 | 0.132616165 | 2.884013651 |
| OASL | 3.536129109 | −3.373570039 | 2.063311525 | 0.16255907 | 5.599440634 |
| PAPPA | 1.773861156 | −2.036309436 | 0.390380692 | −0.262448279 | 2.164241848 |
| PARP10 | 1.326615304 | −2.536442296 | 0.402346977 | −1.209826991 | 1.728962281 |
| PARP12 | 2.850604542 | −3.023002311 | 0.766021002 | −0.172397769 | 3.616625545 |
| PARP14 | 2.583199759 | −2.668276254 | 0.521446407 | −0.085076495 | 3.104646166 |
| PARP9 | 2.126994093 | −1.978563435 | 0.430816142 | 0.148430659 | 2.557810235 |
| PARP9 | 3.27501628 | −2.702171197 | 0.444946635 | 0.572845083 | 3.719962915 |
| PCBP3 | 1.037949803 | −1.019127778 | 0.201291636 | 0.018822025 | 1.239241439 |
| PDK4 | 1.618120933 | −1.329005545 | 0.022234341 | 0.289115388 | 1.640355274 |
| PDPN | 1.451948088 | −1.541326521 | −0.284732802 | −0.089378433 | 1.167215286 |
| PHF11 | 1.569022139 | −2.232289429 | 0.650930764 | −0.663267289 | 2.219952903 |
| PHF11 | 1.486247098 | −2.46187233 | 0.512439235 | −0.975625232 | 1.998686333 |
| PHLDA3 | 1.183373039 | −1.520391317 | 0.197956852 | −0.337018278 | 1.381329891 |
| PLA2G4C | 1.014228181 | −1.304151409 | 0.071454308 | −0.289923228 | 1.085682489 |
| PLEKHA4 | 1.637450704 | −1.3962661 | 1.317454769 | 0.241184604 | 2.954905473 |
| PLXNB1 | 1.022415626 | −1.365163177 | −0.096237354 | −0.342747551 | 0.926178272 |
| PPAP2A | 1.209105487 | −1.07983494 | 0.545678749 | 0.129270547 | 1.754784236 |
| PRIC285 | 3.012033847 | −2.562869089 | 0.854090164 | 0.449164758 | 3.866124011 |
| PSMB8 | 1.741409023 | −2.133472048 | 0.71176636 | −0.392063025 | 2.453175382 |
| PSMB8 | 1.866869714 | −2.200415524 | 0.645884897 | −0.33354581 | 2.512754611 |
| PSMB8 | 2.084184811 | −2.268354645 | 0.5538343 | −0.184169834 | 2.638019111 |
| PSMB9 | 2.430860946 | −2.965037107 | 0.939217969 | −0.534176161 | 3.370078914 |
| PSME1 | 1.487094034 | −1.159108877 | 0.270285521 | 0.327985157 | 1.757379556 |
| PSME2 | 1.33182787 | −1.465882921 | 0.446427126 | −0.134055051 | 1.778254996 |
| PSTPIP2 | 1.09063831 | −1.179957984 | 0.435242738 | −0.089319674 | 1.525881048 |
| PTGER2 | 1.000183179 | −1.17556292 | −0.202348968 | −0.175379741 | 0.797834212 |
| PTGES | 1.148907799 | −1.41021311 | 0.290393152 | −0.261305311 | 1.439300951 |
| PTGFR | 1.449331827 | −1.001601653 | 0.144372433 | 0.447730174 | 1.593704259 |
| PTGFR | 1.603159628 | −1.169956502 | 0.207292196 | 0.433203126 | 1.810451824 |
| PTX3 | 2.263730112 | −2.441375044 | 0.922458958 | −0.177644932 | 3.18618907 |
| PYGB | 1.107233391 | −2.015216504 | −0.118331933 | −0.907983113 | 0.988901458 |
| RARRES3 | 2.25204439 | −2.047163548 | 1.253186958 | 0.204880842 | 3.505231348 |
| RBCK1 | 1.149568733 | −1.372120151 | 0.357340897 | −0.222551418 | 1.50690963 |
| RBM43 | 1.286746579 | −1.355757178 | 0.307342528 | −0.069010599 | 1.594089107 |
| RCAN1 | 1.356269615 | −2.044901472 | 0.590666079 | −0.688631857 | 1.946935694 |
| RELB | 1.327414196 | −1.197089288 | 0.265979581 | 0.130324908 | 1.593393777 |
| RNF213 | 1.026257639 | −1.741511934 | −0.290717605 | −0.715254295 | 0.735540034 |
| RSAD2 | 2.054673683 | −2.109763462 | 2.325557335 | −0.055089779 | 4.380231018 |
| RTN1 | 1.418819362 | −1.193126029 | −0.052438051 | 0.225693333 | 1.36638131 |
| RTP4 | 1.880595259 | −1.968387483 | 1.165511333 | −0.087792223 | 3.046106592 |
| S1PR3 | 2.559224128 | −2.033491398 | 1.123858575 | 0.52573273 | 3.683082702 |
| SAA1 | 3.85141175 | −3.821734188 | 0.256140691 | 0.029677562 | 4.107552441 |
| SAA1 | 1.898438098 | −2.092755962 | 0.278410761 | −0.194317864 | 2.17684886 |
| SAA2 | 1.037522145 | −1.01522487 | 0.248605491 | 0.022297274 | 1.286127636 |
| SAMD9 | 2.925593069 | −2.794870287 | 1.396975715 | 0.130722782 | 4.322568785 |
| SAMD9L | 2.369767909 | −2.404963489 | 1.249533389 | −0.035195579 | 3.619301299 |
| SCHIP1 | 1.175607634 | −1.985056899 | 0.060788621 | −0.809449265 | 1.236396254 |
| SEMA4B | 1.085951137 | −1.750329849 | 0.149020906 | −0.664378712 | 1.234972043 |
| SERPINA3 | 4.119662196 | −2.227495953 | −0.287856775 | 1.892166243 | 3.831805421 |
| SERPINE2 | 1.442986361 | −1.370783037 | 0.204105894 | 0.072203324 | 1.647092255 |
| SERPING1 | 1.693843482 | −1.460555279 | 1.17423948 | 0.233288203 | 2.868082962 |
| SESN1 | 1.939615386 | −1.536312348 | −0.112046912 | 0.403303038 | 1.827568474 |
| SHISA5 | 1.977851326 | −1.756748253 | −0.28305778 | 0.221103073 | 1.694793547 |
| SLC15A3 | 3.130576286 | −3.64382322 | 0.779102407 | −0.513246933 | 3.909678693 |
| SLC22A18 | 1.2134566 | −1.443898771 | −0.050527818 | −0.23044217 | 1.162928783 |
| SLC2A5 | 1.72003983 | −1.822730899 | 0.79755969 | −0.102691069 | 2.51759952 |
| SLC39A14 | 1.097600298 | −1.749617299 | 0.470029151 | −0.652017 | 1.567629449 |
| SLC39A8 | 1.244476955 | −1.095426078 | 0.863021155 | 0.149050877 | 2.10749811 |
| SLC44A1 | 1.71490211 | −1.104904122 | 0.779718309 | 0.609997987 | 2.494620419 |
| SLC7A11 | 1.166852082 | −1.137370507 | 1.259609808 | 0.029481575 | 2.42646189 |
| SLC7A2 | 2.259398922 | −2.39407 | −0.106968828 | −0.134671078 | 2.152430093 |
| SNAI2 | 1.342068773 | −2.049114848 | 0.214733442 | −0.707046075 | 1.556802215 |
| SOD2 | 5.212222862 | −4.543679151 | 1.537353539 | 0.668543711 | 6.749576401 |
| SOD2 | 4.995584404 | −3.780434297 | 0.347061658 | 1.215150107 | 5.342646062 |
| SOD2 | 1.60136037 | −1.58646149 | −0.064257692 | 0.01489888 | 1.537102678 |
| SP100 | 2.100236314 | −2.018687379 | 1.188181794 | 0.081548935 | 3.288418108 |
| SP100 | 1.508601396 | −1.642600221 | 1.176024656 | −0.133998825 | 2.684626052 |
| SP110 | 2.518950711 | −2.846716622 | 1.137414514 | −0.327765911 | 3.656365225 |
| SP110 | 1.720088434 | −1.882249168 | 1.055346467 | −0.162160733 | 2.775434901 |
| SP110 | 2.690392001 | −2.892516229 | 1.103030741 | −0.202124228 | 3.793422743 |
| SP110 | 1.310024912 | −1.487091031 | 0.740242611 | −0.177066119 | 2.050267523 |
| SPATA18 | 1.944042704 | −1.862961372 | −0.20921297 | 0.081081332 | 1.734829734 |
| SPTLC3 | 1.188352855 | −1.296558987 | 0.230797274 | −0.108206132 | 1.419150129 |
| SPTLC3 | 1.17708351 | −1.407741922 | −0.378437155 | −0.230658412 | 0.798646355 |
| SRGN | 1.075062288 | −1.878809962 | 0.204158948 | −0.803747674 | 1.279221236 |
| SRGN | 1.238744661 | −1.68310726 | 0.209977919 | −0.444362599 | 1.44872258 |
| SSBP2 | 1.005366935 | −1.261257011 | 0.350878869 | −0.255890076 | 1.356245804 |
| SSH2 | 1.199702614 | −1.247618181 | −0.311161918 | −0.047915566 | 0.888540696 |
| STAT1 | 3.947853023 | −2.871457346 | 0.837427127 | 1.076395677 | 4.785280151 |
| STAT1 | 3.99689458 | −2.848826485 | 0.468407551 | 1.148068096 | 4.465302131 |
| STAT1 | 2.906304539 | −2.585014232 | −0.093155195 | 0.321290307 | 2.813149344 |
| STAT2 | 1.915817586 | −2.686061344 | 0.191027908 | −0.770243758 | 2.106845494 |
| STC1 | 1.0292146 | −1.457999055 | 0.794608722 | −0.428784454 | 1.823823322 |
| STOM | 2.16750243 | −2.265579191 | 0.636207371 | −0.098076761 | 2.803709801 |
| STXBP6 | 1.685192125 | −2.205008349 | −0.140264617 | −0.519816224 | 1.544927508 |
| STXBP6 | 1.357533381 | −1.548702316 | −1.014370584 | −0.191168935 | 0.343162797 |
| SUSD1 | 1.167809054 | −1.215356084 | 0.502018478 | −0.04754703 | 1.669827532 |
| SUSD2 | 1.995586666 | −1.30123156 | −1.843821737 | 0.694355106 | 0.151764929 |
| TAP1 | 2.554918275 | −3.250566846 | 0.704354101 | −0.695648571 | 3.259272376 |
| TAP2 | 1.139829115 | −1.104732199 | 1.295486422 | 0.035096916 | 2.435315537 |
| TAP2 | 1.495730318 | −1.568017857 | −0.063687025 | −0.072287539 | 1.432043293 |
| TAPBP | 1.779590119 | −1.814839188 | 0.46938487 | −0.035249069 | 2.248974988 |
| TCEA3 | 1.187385692 | −1.689879617 | −0.116757872 | −0.502493925 | 1.07062782 |
| TGFBR3 | 1.642867893 | −1.213035396 | 0.441813674 | 0.429832497 | 2.084681566 |
| TLR3 | 1.00307105 | −1.066022034 | 1.561205837 | −0.062950984 | 2.564276887 |
| TMEM140 | 2.60512035 | −2.638936113 | 0.985994997 | −0.033815763 | 3.591115347 |
| TMEM179B | 1.063133997 | −1.059209078 | 0.48556917 | 0.003924918 | 1.548703167 |
| TNFAIP2 | 1.009483894 | −1.432893589 | 0.055437207 | −0.423409695 | 1.0649211 |
| TNFAIP3 | 2.222657286 | −2.600109658 | 0.274980522 | −0.377452372 | 2.497637808 |
| TNFAIP6 | 3.800322521 | −2.175271279 | 1.295987256 | 1.625051242 | 5.096309777 |
| TNFRSF14 | 1.212245798 | −2.570296708 | 0.566392867 | −1.35805091 | 1.778638665 |
| TNFRSF6B | 1.314356831 | −1.602258212 | 0.008270152 | −0.287901381 | 1.322626983 |
| TNFRSF6B | 1.045278227 | −1.221948387 | 0.037804436 | −0.176670161 | 1.083082663 |
| TNFRSF6B | 1.606266779 | −1.778662824 | −0.018117171 | −0.172396045 | 1.588149607 |
| TNFSF13B | 1.646466367 | −1.528604316 | 2.13086148 | 0.117862051 | 3.777327847 |
| TNFSF13B | 1.499970062 | −1.427861244 | 2.150984925 | 0.072108818 | 3.650954987 |
| TP53I3 | 1.219126295 | −1.936048428 | 0.187892187 | −0.716922133 | 1.407018482 |
| TP53INP1 | 1.372265015 | −1.110614071 | −0.560185532 | 0.261650944 | 0.812079483 |
| TRIM21 | 1.94378019 | −1.315572815 | 0.970781411 | 0.628207375 | 2.9145616 |
| TRIM22 | 2.23643209 | −2.500884935 | −0.01884083 | −0.264452845 | 2.21759126 |
| TRIM25 | 2.180788981 | −2.44717143 | 0.507762266 | −0.266382449 | 2.688551247 |
| TRIM5 | 1.356054889 | −1.320968917 | 1.331111668 | 0.035085972 | 2.687166557 |
| TRIM55 | 1.011150302 | −1.049617761 | 0.204866799 | −0.038467459 | 1.216017102 |
| TSC22D3 | 1.675169239 | −1.64108393 | 0.195597878 | 0.034085309 | 1.870767117 |
| TSC22D3 | 2.367185894 | −1.913270465 | 0.277087408 | 0.45391543 | 2.644273302 |
| TSC22D3 | 2.253212406 | −1.519040364 | 0.175595871 | 0.734172041 | 2.428808277 |
| TTC39B | 1.177973441 | −1.145548414 | 1.195730435 | 0.032425027 | 2.373703876 |
| TYMP | 3.590408302 | −4.248591981 | 0.616829013 | −0.658183679 | 4.207237316 |
| TYMP | 1.807874606 | −1.828855461 | 0.992455865 | −0.020980856 | 2.800330471 |
| TYMP | 2.13163775 | −2.186708951 | 1.227113406 | −0.055071201 | 3.358751156 |
| TYMP | 1.899181412 | −1.941140272 | 0.995147713 | −0.04195886 | 2.894329125 |
| UBA7 | 1.723362397 | −2.412154439 | 0.569362394 | −0.688792042 | 2.29272479 |
| UBE2L6 | 2.264152884 | −3.092070504 | 0.57213622 | −0.82791762 | 2.836289104 |
| UBE2L6 | 1.959635831 | −2.107710194 | 0.942889832 | −0.148074363 | 2.902525663 |
| UGP2 | 1.152318593 | −1.131727121 | 0.108407345 | 0.020591472 | 1.260725938 |
| UNC93B1 | 1.230652435 | −1.872083002 | 0.936282671 | −0.641430567 | 2.166935106 |
| USP18 | 2.316759582 | −2.22518459 | 0.972883677 | 0.091574992 | 3.289643259 |
| VCAM1 | 2.680275193 | −2.786083447 | 0.630957412 | −0.105808254 | 3.311232604 |
| VWCE | 2.836246852 | −3.054134611 | −0.118830018 | −0.217887759 | 2.717416834 |
| XAF1 | 2.657844238 | −3.488687703 | 0.613210061 | −0.830843465 | 3.2710543 |
| XAF1 | 1.360588979 | −1.759772476 | 0.788471377 | −0.399183497 | 2.149060356 |
| XPC | 1.949606716 | −2.428503055 | 0.257466175 | −0.478896339 | 2.207072891 |
| YPEL3 | 1.246536785 | −1.235396214 | −0.268728988 | 0.01114057 | 0.977807797 |
| ZBTB16 | 1.972537335 | −1.863255579 | 0.481865817 | 0.109281756 | 2.454403152 |
| ZBTB16 | 1.99291856 | −2.195343242 | 0.412011223 | −0.202424682 | 2.404929783 |
| ZC3H12A | 1.664514842 | −1.372941613 | 0.519679139 | 0.291573229 | 2.184193981 |
| ZFHX3 | 1.106012558 | −1.699672846 | 0.50234344 | −0.593660287 | 1.608355998 |
| ZNFX1 | 1.751304649 | −2.100029966 | 0.809909136 | −0.348725317 | 2.561213785 |

GFP v. NoLenti (Fc) = log₂ fold change lenti-GFP versus uninfected
Full_v. GFP (Fc) = log₂ fold change lenti-DUX4-fl versus lenti-GFP
Short_v. GFP (Fc) = log₂ fold change lenti-DUX4-s versus lenti-GFP
Full v._NoLenti (Fc) = log₂ fold change lenti-DUX4-fl versus uninfected
Short_vs. NoLenti (Fc) = log₂ fold change lenti-DUX4-s versus uninfected

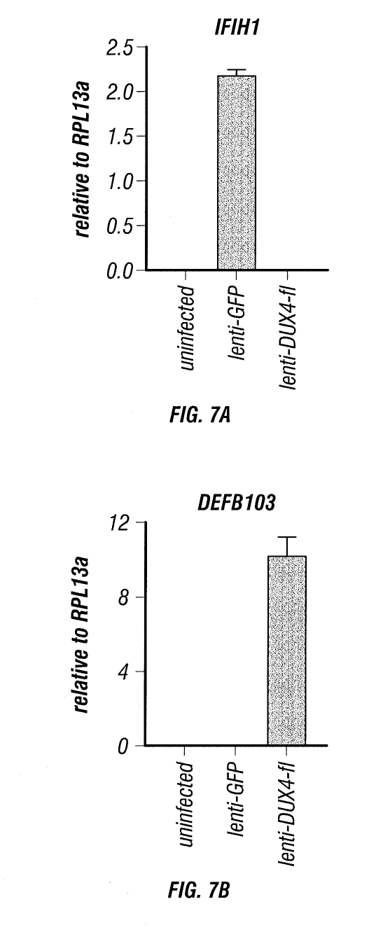

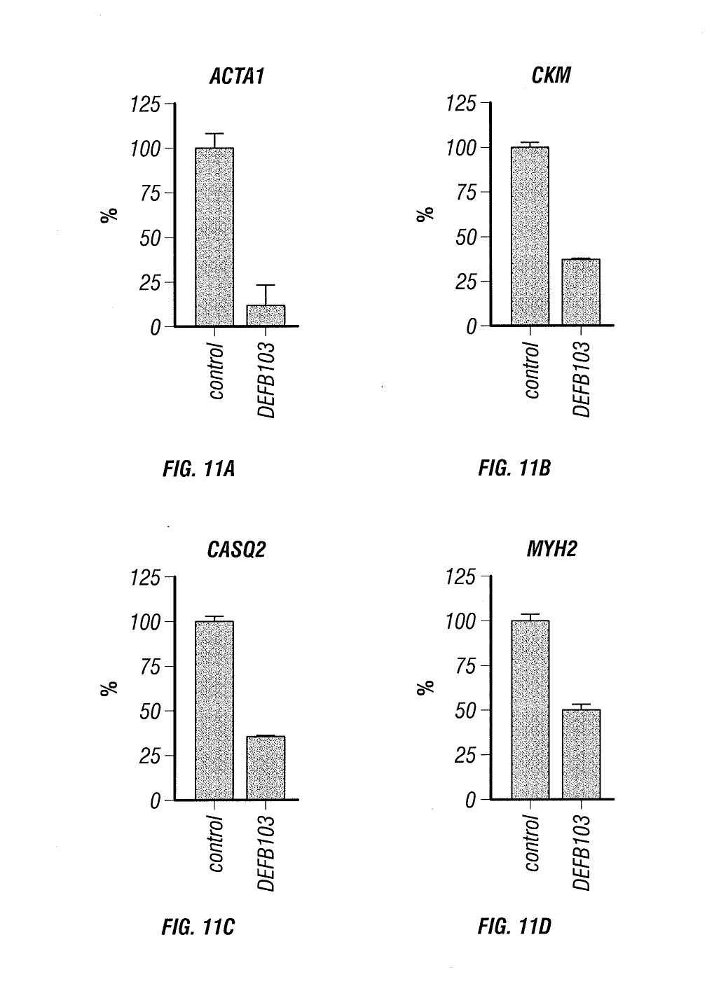

FIGS. 7-11 show the results of real-time PCR analysis demonstrating that DEFB103 inhibits the innate immune response to viral infection and inhibits muscle differentiation. Values shown in FIGS. 7-11 represent mean+/−SD from triplicates and are either expressed as relative to internal standard RPL13a or as a percentage relative to control condition after being normalized to RPL13a.

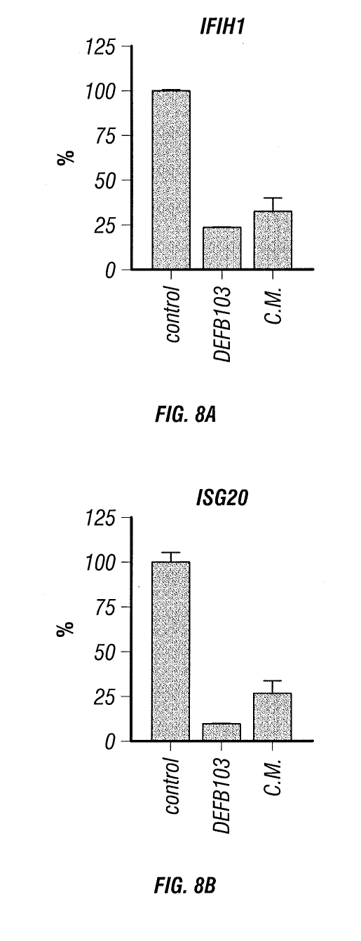

The RT-qPCR results validated that lenti-GFP, lenti-DUX4-s and multiple other lentivirus constructs induced the innate immune response in myoblasts, whereas similar titers of lenti-DUX4-fl did not (see FIG. 7A, and data not shown). Additionally, supernatant from DUX4-fl infected cells (CM) reduced the induction of these genes by lenti-GFP (see FIG. 8), indicating that a secreted factor induced by DUX4-fl could mediate this suppressive effect.

DUX4-fl robustly induced expression of DEFB103A/B (β-defensin 3) (set forth as SEQ ID NO:178), as shown in FIG. 7B, which has been shown to inhibit the transcription of pro-inflammatory genes in TLR4-stimulated macrophages (Semple et al., 2011). Indeed, addition of DEFB103 peptide also inhibited the induction of the innate immune response to lenti-GFP when added to the muscle cells at the time of infection (see FIG. 8), but did not prevent viral entry and transduction as measured by copies of viral integrants in the genome and levels of GFP mRNA expressed (data not shown). Thus, DUX4 can prevent the innate immune response to viral infection in skeletal muscle cells, at least in part, through the transcriptional induction of DEFB103.

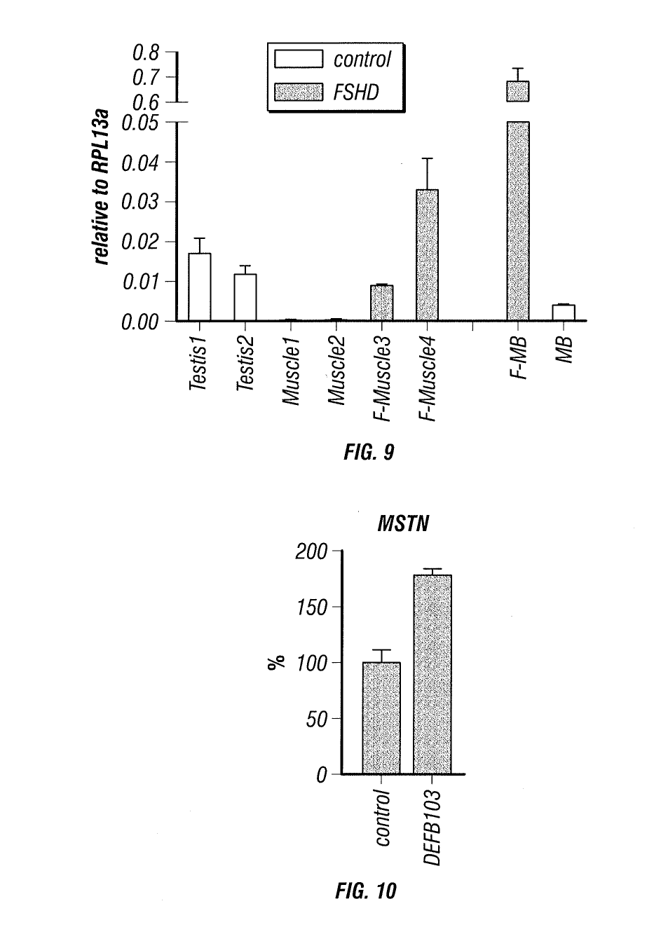

Like other DUX4-regulated genes, endogenous expression of DEFB103 was detected in FSHD cultured muscle cells, FSHD biopsies, and in healthy testes, but little to none was seen in control skeletal muscle, as shown in FIG. 9.

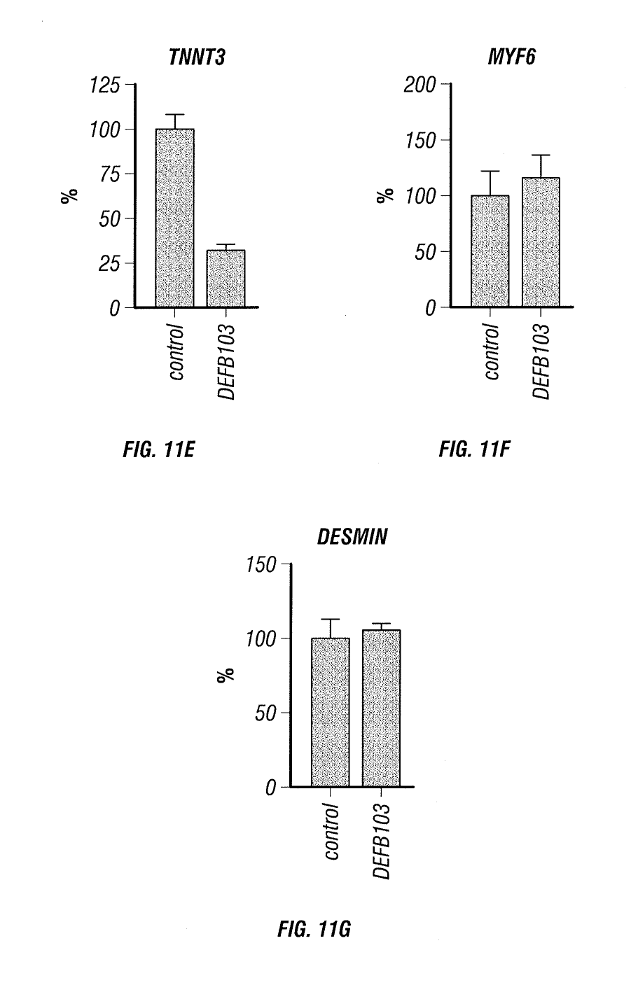

DEFB103 has been previously shown to bind to the CCR6, CCR2, and melanocortin receptors and to be an antagonist ligand for the CXCR4 receptor, which is important for muscle cell migration and differentiation (Candille et al., 2007, Feng et al., 2006; Jin et al., 2010; Yang et al., 1999). To determine whether DEFB103 could affect myoblasts or muscle differentiation, the inventors treated cultured control human muscle cells with DEFB103 peptide at concentrations considered to be physiological (0.5-1.0 ug/ml) (see Midorikawa et al., 2003; Semple et al., 2011), and assessed changes with gene expression arrays. Based on a 2-fold change threshold, DEFB103 did not alter the expression of any genes in myoblasts, although it is of interest that myostatin was upregulated approximately 50% and RT-qPCR confirmed that DEFB103 increased the mRNA for myostatin in myoblasts (as shown in FIG. 11). In contrast, exposing differentiating muscle cells to DEFB103 reduced the expression of 44 genes relative to the untreated control, the majority of which were genes associated with muscle differentiation, as shown below in TABLE 14.

TABLE 14
DEFB103 suppresses the induction of skeletal
muscle differentiation genes.

| Symbol | log₂ FC defMT/MT | log₂ FC MT/MB |
|---|---|---|
| ACTA1 | −2.65 | 5.42 |
| MYH8 | −2.62 | 4.58 |
| MYH3 | −2.20 | 6.93 |
| CASQ2 | −2.00 | 3.49 |
| CKM | −1.90 | 3.28 |
| MYL4 | −1.86 | 4.59 |
| SMPX | −1.76 | 2.92 |
| MYH7 | −1.73 | 2.11 |
| CACNG1 | −1.58 | 2.51 |
| TNNT3 | −1.55 | 2.85 |
| MYLPF | −1.53 | 6.54 |
| TNNT3 | −1.52 | 2.89 |
| ENO3 | −1.51 | 3.38 |
| MYBPH | −1.49 | 5.13 |
| TNNC2 | −1.49 | 2.29 |
| LOC389827 | −1.48 | 2.30 |
| ENO3 | −1.47 | 2.51 |
| TNNC1 | −1.45 | 5.92 |
| TPM2 | −1.41 | 3.53 |
| HRC | −1.40 | 3.31 |
| LOC389827 | −1.38 | 2.20 |
| HES6 | −1.24 | 3.80 |
| VASH2 | −1.22 | 1.78 |
| MYOM1 | −1.20 | 1.70 |
| MYL1 | −1.18 | 5.71 |
| AIF1L | −1.16 | 1.95 |
| CKB | −1.16 | 2.98 |
| CTGF | −1.12 | 0.82 |
| MYL1 | −1.11 | 5.58 |
| HBEGF | −1.10 | 2.07 |
| PRAGMIN | −1.10 | 1.38 |
| FOLR1 | −1.10 | 1.45 |
| ZFP106 | −1.09 | 1.55 |
| MYL4 | −1.08 | 1.98 |
| SMYD1 | −1.07 | 2.42 |
| ARPP-21 | −1.06 | 2.86 |
| CYP2J2 | −1.05 | 1.39 |
| ATP2A2 | −1.04 | 2.48 |
| HFE2 | −1.04 | 2.89 |
| RASSF4 | −1.03 | 2.49 |
| IL32 | −1.03 | 1.85 |
| FOLR1 | −1.03 | 1.38 |
| LMCD1 | −1.03 | 1.24 |
| MYL6B | −1.00 | 1.59 |
| TNNT1 | −1.00 | 2.27 |
| NDRG1 | 1.01 | −0.09 |
| ANGPTL4 | 1.21 | −0.76 |
| DKK1 | 1.22 | −3.56 |
| MME | 1.27 | −1.36 |
| AKR1C2 | 1.41 | 2.32 |
| PLIN2 | 1.61 | −0.76 |
| MT1X | 1.65 | −1.50 |
| HMOX1 | 1.66 | −0.15 |
| PLIN2 | 1.79 | −0.93 |

log 2 FC DefMT/MT is the log 2 ratio of expression in DEF103 treated muscle cells compared to control muscle cells
log 2 FC MT/MB is the log 2 ratio of expression in differentiated muscle cells to myoblasts

As shown in FIG. 11, RT-qPCR on select genes (ACTA1, CKM, CASQ2, MYH2 and TNNT3) validated the array results. Therefore, DEFB103 activates the expression of myostatin in myoblasts and inhibits the expression of genes necessary for normal muscle differentiation. Therefore, DUX4-mediated expression of DEFB103 in FSHD muscle can modulate the innate immune response to retroviral infection and can inhibit myogenic differentiation.

The induction of DEFB103 by DUX4 might influence both the adaptive and the innate immune response. DEFB103 can have a pro-inflammatory role in the adaptive immune response and can act as a chemo-attractant for monocytes, lymphocytes and dendritic cells (Lai and Gallo, 2009). In this regard, it might enhance an adaptive immune response to germline antigens expressed in FSHD muscle. Though traditionally known for its role in antimicrobial defense (Sass et al., 2010), DEFB103 has been shown to suppress the innate immune response to LPS and TLR4 stimulation in macrophages (Semple et al., 2011; Semple et al., 2010). DEFB103 has also been shown to be an antagonistic ligand of the CXCR4 receptor (Feng et al., 2006), which is important for muscle migration, regeneration, and differentiation (Griffin et al., 2010; Melchionna et al., 2010).

### Discussion of Results

As described herein, the inventors have identified genes regulated by DUX4-fl and show that they are expressed at readily detectable levels in FSHD skeletal muscle, both cell lines and muscle biopsies, but not in control tissues, providing direct support for the model that misexpression of DUX4-fl is a causal factor for FSHD. The genes regulated by DUX4-fl suggest several specific mechanisms for FSHD pathophysiology.

In the Examples provided herein the inventors have demonstrated that DEFB103 inhibited the innate immune response to lentiviral infection in skeletal muscle cells, modestly induced myostatin in myoblasts, and impaired muscle cell differentiation. Therefore, while not wishing to be bound by any particular theory, DEFB103 may contribute to FSHD pathology by modulating the adapative and innate immune response, as well as through inhibiting muscle differentiation.

Reactivation of retroelements can result in genomic instability (Belancio et al., 2010) and transcriptional deregulation (Schulz et al., 2006). Therefore, DUX4 activation of MaLR transcripts might directly contribute to FSHD pathophysiology. It is interesting that DUX4 both activates retroelement transcription and suppresses the virally induced innate immune response. Although the inventors have shown that DEFB103 can substitute for DUX4 to suppress the innate immune response, products of retroelements and endogenous retroviruses may do the same and, thus, the DUX4-mediated suppression of the innate immune response might be multi-factorial. Since DEFB103 is also expressed in the testis, it is interesting to consider whether the role of DUX4 in the germline might include a simultaneous activation of retroelement transcription and suppression of the innate immune response to those transcripts.

DUX4 regulated targets also include genes involved in RNA splicing, developmentally regulated components of the Pol II transcription complex, and ubiquitin-mediated protein degradation pathways, all of which may have pathophysiological consequences. For example, DUX4 is known to induce apoptosis and inhibit myogenesis in muscle cells In this regard, other genes have been identified as candidates for FSHD. For example, FRG1 expression has been reported to be elevated in FSHD muscle (Gabellini et al., 2002) and FRG1 transgenic mice display a muscular dystrophy phenotype (Gabellini et al., 2006). It is interesting the FRG1 is reported to alter RNA splicing in FSHD muscle (Gabellini et al., 2006 supra) and that the inventors' study shows that DUX4-fl also alters the expression of many genes that regulate splicing and RNA processing. It will be important to determine the relative contributions of DUX4 and FRG1 to FSHD pathophysiology; however, the human genetics shows a convincing linkage to polymorphisms necessary for the polyadenylation of the DUX4 mRNA (Lemmers et al., 2010), indicating that DUX4 mRNA is a necessary component of the disease. Therefore, agents that reduce the activity of DUX4, either by eliminating its expression in the muscle cells as the inventors have done in vitro with an siRNA or by introducing a dominant negative, such as the DUX4-s splice form are believed to be useful as therapeutic agents for treatment of subjects suffering from FSHD and/or for prevention of symptoms related to FSHD.

In conclusion, these data support the model that inappropriate expression of DUX4 plays a causal role in FSHD skeletal muscle pathophysiology by activating germline gene expression, endogenous retrotransposons, and suppressors of differentiation in skeletal muscle. The set of genes robustly upregulated by DUX4 in FSHD skeletal muscle are candidate biomarkers because they are absent in control muscle and easily detected in FSHD1 and FSHD2 muscle. Furthermore, some target genes encode secreted proteins, which offer the potential for developing blood tests to diagnose FSHD or monitor response to interventions. Beyond their utilities as candidate biomarkers, the DUX4 targets identified in this study point to specific mechanisms of disease and may help guide the development of therapies for FSHD.

Accordingly, in view of the data demonstrating that DEFB103 blocks myogenesis, the therapeutic indication would be to neutralize or block DEFB103 in FSHD. Therefore in some embodiments, the invention provides DEFB103 inhibitory agents and methods of using DEFB103 inhibitors to treat FSHD subjects and to ameliorate or prevent symptoms associated with FSHD by promoting muscle regeneration/differentiation.

In another embodiment, the invention provides methods of treating a subject suffering from a sarcopenias or other muscular dystrophy by administering an amount of DEFB103 inhibitory agent effective to facilitate normal muscle regeneration/differentiation.

### Example 9

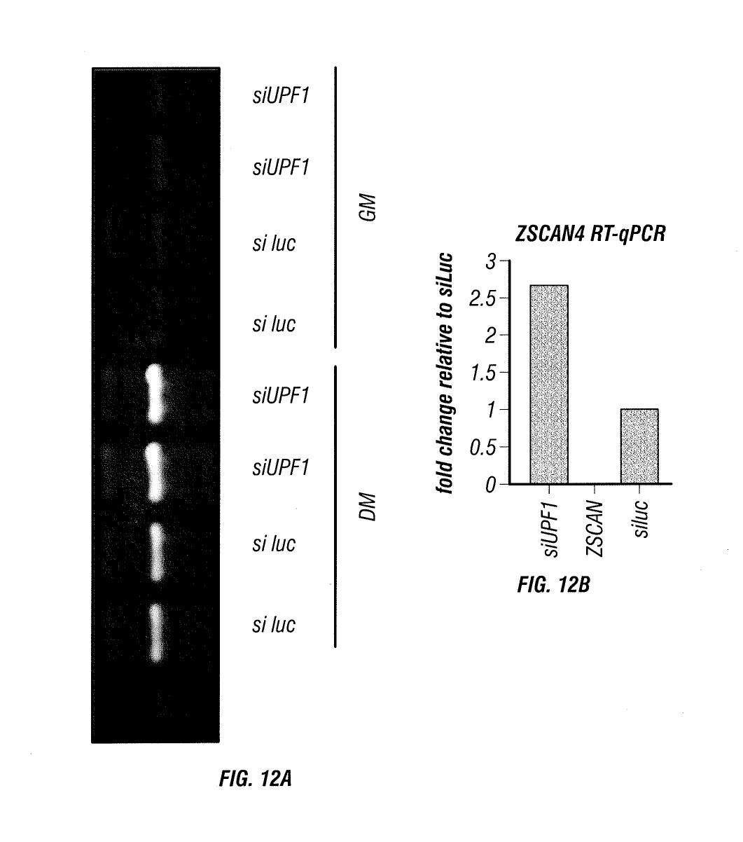

siRNA knock-down of UPF1 results in an increase of DUX4 mRNA and an increase in the expression of a DUX4 target gene ZSCAN4. Consistent with the higher DUX4 mRNA in cells with UPF1 knock-down, there is a higher abundance of the ZSCAN4 target gene in the cells with the DUX4 knock-down (FIG. 12A-12B).

Method for transfection of myoblasts with siUPF1 (siRNA against UPF1): Human FSHD myoblasts were plated for transfection at 30% confluency in F10 media containing 20% FBS and no antibiotics. siRNA targeting siUPF1 (ThermoScientific) was transfected at 100 nM final concentration using Lipofectamine 2000 according to manufacturers protocol. siLuciferase was used as a negative control. Following overnight incubation of cells with transfection complexes the cells were washed with PBS and fed fresh growth media: F10 media, 20% FBS, dexamethasone (1 uM) and fgf (10 ng/ml). When cultures reached confluency growth media was replaced with serum free differentiation media (F10 media, insulin and transferrin at 10 ug/ml each) and culture continued for 48 hours. Total RNA was isolated using Qiagen RNeasy columns and analysis for expression of Dux4 mRNA and for target gene activation was performed.

The sequence of siUPF1 used herein is GAUGCAGUUCCGCUCCAUUUU (sense) (SEQ ID NO:202) (See Kim et al.). The cDNA sequence of human UPF1 is NM_{—}002911 (SEQ ID NO:203).

APPENDIX
TABLE 1: Expression Array Analysis of DUX4-fl and DUX4-s in cultured human skeletal muscle

| | Symbol | Refseq* | Full.fc | Short.fc | Full.pval | Short.pval | Full.fdr | Short.fdr |
|---|---|---|---|---|---|---|---|---|
| 1. | RFPL1S | NR_002727.1 | 8.395820858 | 0.114675803 | 4.68E−27 | 0.239049748 | 0 | 0.42494 |
| 2. | LOC643263 | XR_016355.1 | 8.345299826 | −0.13296007 | 5.16E−27 | 0.174365432 | 0 | 0.34164 |
| 3. | RFPL4B | NM_001013734.2 | 8.340345819 | −0.10307784 | 5.13E−28 | 0.233239884 | 0 | 0.41777 |
| 4. | LOC390031 | XM_372343.1 | 8.330613566 | −0.10971721 | 5.02E−28 | 0.204581737 | 0 | 0.38176 |
| 5. | ZSCAN4 | NM_152677.1 | 8.321990102 | 0.04366422 | 1.94E−28 | 0.589332902 | 0 | 0.75086 |
| 6. | LOC340970 | XR_038494.1 | 8.315993278 | 0.03296229 | 3.20E−28 | 0.69048443 | 0 | 0.82273 |
| 7. | LOC136157 | XM_069743.3 | 8.298510216 | 0.05270893 | 1.98E−27 | 0.561916557 | 0 | 0.73064 |
| 8. | LOC643445 | XR_038080.1 | 8.249957558 | 0.078820982 | 1.44E−28 | 0.322618619 | 0 | 0.51927 |
| 9. | LOC729458 | XM_001130308.2 | 8.246687197 | 0.008125932 | 2.30E−27 | 0.928539738 | 0 | 0.96551 |
| 10. | LOC653192 | XM_926437.2 | 8.228018909 | −0.03931919 | 2.48E−27 | 0.665571629 | 0 | 0.80599 |
| 11. | LOC645669 | XM_928680.1 | 8.202022481 | 0.087540869 | 1.85E−27 | 0.331908052 | 0 | 0.52852 |
| 12. | LOC391769 | XM_001713901.1 | 8.189552468 | 0.151962984 | 3.39E−27 | 0.109385798 | 0 | 0.24599 |
| 13. | LOC196120 | XM_114987.3 | 8.178925427 | 0.051907662 | 2.42E−27 | 0.566127782 | 0 | 0.73383 |
| 14. | LOC651308 | XM_940443.1 | 8.168661444 | 0.039056723 | 4.84E−25 | 0.740883199 | 0 | 0.85586 |
| 15. | RFPL3 | NM_001098535.1 | 8.144474769 | −0.06033097 | 9.29E−29 | 0.430802078 | 0 | 0.62198 |
| 16. | PRAMEF1 | NM_023013.1 | 8.072400408 | 0.069102721 | 3.19E−27 | 0.44691755 | 0 | 0.63632 |
| 17. | LOC100134199 | XM_001719549.1 | 8.048036849 | 0.032523272 | 6.76E−28 | 0.695970658 | 0 | 0.82645 |
| 18. | SPRYD5 | NM_032681.1 | 8.044967325 | −0.07701669 | 5.44E−28 | 0.353686738 | 0 | 0.55026 |
| 19. | LOC284428 | XM_208203.5 | 8.022522551 | −0.09940864 | 1.38E−26 | 0.309802653 | 0 | 0.50524 |
| 20. | LOC642362 | XM_925891.1 | 8.015825025 | −0.01469809 | 1.66E−27 | 0.865315092 | 0 | 0.92997 |
| 21. | KHDC1L | NM_001126063.2 | 8.012411091 | −0.07068267 | 1.06E−27 | 0.407798804 | 0 | 0.60161 |
| 22. | LOC653656 | XM_928688.3 | 7.897231482 | −0.14187285 | 5.40E−28 | 0.090015441 | 0 | 0.21361 |
| 23. | TRIM48 | NM_024114.2 | 7.880137061 | −0.07953563 | 5.54E−26 | 0.438422117 | 0 | 0.62889 |
| 24. | LOC653657 | XM_928697.2 | 7.856575803 | 0.186023041 | 3.03E−27 | 0.044025347 | 0 | 0.12468 |
| 25. | PRAMEF12 | NM_001080830.1 | 7.801903788 | 0.126872497 | 1.84E−25 | 0.244937761 | 0 | 0.43179 |
| 26. | LOC441584 | XM_497258.1 | 7.781378819 | −0.14419106 | 4.75E−27 | 0.115629739 | 0 | 0.25615 |
| 27. | LOC730974 | XR_037751.1 | 7.715075519 | 0.023836761 | 9.06E−26 | 0.815803599 | 0 | 0.90182 |
| 28. | PRAMEF7 | NM_001012277.1 | 7.631155888 | 0.106015999 | 1.22E−27 | 0.201113017 | 0 | 0.37739 |
| 29. | MBD3L2 | NM_144614.2 | 7.622770725 | 0.026835566 | 3.46E−26 | 0.780460765 | 0 | 0.88063 |
| 30. | LOC440040 | XM_495873.4 | 7.533852122 | 0.081279966 | 2.79E−27 | 0.336680469 | 0 | 0.53366 |
| 31. | CCNA1 | NM_003914.2 | 7.525825564 | 1.883773429 | 1.10E−26 | 4.89E−15 | 0 | 0 |
| 32. | PRAMEF13 | XM_001713933.1 | 7.421574077 | 0.11785555 | 3.37E−27 | 0.166851976 | 0 | 0.33175 |
| 33. | LOC342900 | XM_001129035.1 | 7.391093477 | 0.131534159 | 4.53E−28 | 0.090162105 | 0 | 0.21383 |
| 34. | LOC340096 | XM_293943.2 | 7.38245832 | −0.07892668 | 9.80E−25 | 0.477985391 | 0 | 0.66308 |
| 35. | PRAMEF5 | NM_001013407.1 | 7.34950535 | 0.105404705 | 3.80E−23 | 0.430635599 | 0 | 0.62189 |
| 36. | RFPL2 | NM_006605.1 | 7.293384138 | 0.031224439 | 3.38E−25 | 0.762925317 | 0 | 0.87012 |
| 37. | PRAMEF9 | NM_001010890.1 | 7.130773908 | −0.07265342 | 7.31E−25 | 0.49226852 | 0 | 0.67472 |
| 38. | LOC100134006 | XM_001725030.1 | 7.08721139 | −0.02089226 | 7.77E−27 | 0.801081744 | 0 | 0.89304 |
| 39. | PRAMEF4 | NM_001009611.1 | 7.060257208 | 0.042304869 | 2.65E−24 | 0.704169906 | 0 | 0.83172 |
| 40. | PRAMEF15 | XM_001713659.1 | 7.000221925 | −0.05867233 | 4.98E−26 | 0.516703891 | 0 | 0.69394 |
| 41. | LOC100131392 | XM_001713681.1 | 6.975776511 | −0.00710376 | 9.12E−25 | 0.945565419 | 0 | 0.97352 |
| 42. | NP | NM_000270.1 | 6.960976026 | 0.227948457 | 4.12E−27 | 0.008382197 | 0 | 0.0338 |
| 43. | LOC399939 | XM_374919.3 | 6.930795087 | −0.06731419 | 9.92E−27 | 0.415983247 | 0 | 0.60893 |
| 44. | LOC642148 | XR_019607.1 | 6.85089804 | 0.135198137 | 8.92E−25 | 0.1950279 | 0 | 0.36981 |
| 45. | LOC729384 | NM_001105522.1 | 6.831960625 | −0.07068137 | 2.20E−27 | 0.350703268 | 0 | 0.54712 |
| 46. | ZNF705A | NM_001004328.1 | 6.831813353 | −0.0605928 | 3.44E−27 | 0.432743467 | 0 | 0.6236 |
| 47. | C6orf148 | NM_030568.2 | 6.759160491 | −0.10455994 | 7.93E−25 | 0.302878889 | 0 | 0.49795 |
| 48. | TRIM49 | NM_020358.2 | 6.551062725 | −0.01702844 | 3.44E−26 | 0.836826254 | 0 | 0.91386 |
| 49. | DEFB103A | NM_001081551.2 | 6.441860402 | 0.065810955 | 1.15E−25 | 0.449659752 | 0 | 0.63905 |
| 50. | PRAMEF2 | NM_023014.1 | 6.439143984 | −0.04937285 | 2.12E−25 | 0.581121856 | 0 | 0.74436 |
| 51. | RFPL1 | NM_021026.2 | 6.264001827 | 0.340220484 | 8.17E−25 | 0.001422993 | 0 | 0.00774 |
| 52. | LOC100133984 | XM_001723079.1 | 6.203778673 | −0.03264954 | 8.08E−25 | 0.722972189 | 0 | 0.84364 |
| 53. | LOC642127 | XM_936272.2 | 6.112037689 | 0.04198888 | 6.46E−24 | 0.677564423 | 0 | 0.81389 |
| 54. | CA2 | NM_000067.1 | 6.091135387 | 0.104613634 | 5.91E−24 | 0.302024566 | 0 | 0.49727 |
| 55. | PRAMEF10 | NM_001039361.1 | 6.063554254 | 0.008756171 | 1.77E−23 | 0.933700957 | 0 | 0.96831 |
| 56. | LOC646698 | XM_929644.2 | 6.012022368 | 0.110541637 | 9.84E−24 | 0.28229693 | 0 | 0.47595 |
| 57. | LOC729516 | XR_038445.1 | 5.954919316 | −0.00368993 | 1.03E−25 | 0.96294883 | 0 | 0.98328 |
| 58. | PRAMEF11 | XM_001714028.1 | 5.93984508 | 0.12170361 | 1.97E−24 | 0.196313298 | 0 | 0.37122 |
| 59. | CSAG3 | NM_001129826.1 | 5.871224381 | 0.090735156 | 6.50E−24 | 0.354078253 | 0 | 0.55061 |
| 60. | PRAMEF6 | NM_001010889.2 | 5.82553958 | −0.06188677 | 8.31E−25 | 0.477094509 | 0 | 0.6623 |
| 61. | LOC391764 | XM_373076.3 | 5.820931052 | 0.121080051 | 1.05E−24 | 0.176502333 | 0 | 0.34469 |
| 62. | TRIM43 | NM_138800.1 | 5.805862854 | 0.023851911 | 1.43E−20 | 0.866762315 | 0 | 0.93058 |
| 63. | LOC391742 | XM_373056.1 | 5.733140049 | 0.159640297 | 1.50E−25 | 0.051475695 | 0 | 0.14059 |
| 64. | LOC391766 | XM_373077.2 | 5.723821554 | −0.05536847 | 3.38E−25 | 0.497562687 | 0 | 0.67933 |
| 65. | ZNF296 | NM_145288.1 | 5.536035027 | 0.1758227 | 9.82E−25 | 0.044329889 | 0 | 0.12539 |
| 66. | SLC34A2 | NM_006424.2 | 5.513611409 | −0.03230882 | 5.77E−22 | 0.777899548 | 0 | 0.87914 |
| 67. | LOC391767 | XM_373078.1 | 5.491772222 | 0.055424445 | 3.46E−21 | 0.658195002 | 0 | 0.80141 |
| 68. | LOC729368 | XM_001130065.2 | 5.416246795 | −0.0600205 | 1.19E−23 | 0.517110941 | 0 | 0.69425 |
| 69. | LOC440563 | NM_001136561.1 | 5.312436177 | 0.070512689 | 3.77E−22 | 0.515651476 | 0 | 0.69305 |
| 70. | LOC646754 | XM_929704.2 | 5.110280465 | −0.14855706 | 3.49E−22 | 0.161275857 | 0 | 0.32367 |
| 71. | LOC654101 | XM_939354.1 | 5.033863949 | 0.094787028 | 5.71E−21 | 0.42384468 | 0 | 0.61572 |
| 72. | LOC729731 | XM_001131140.1 | 5.007248294 | 0.098807532 | 1.46E−23 | 0.258865952 | 0 | 0.44828 |
| 73. | HIST2H3A | NM_001005464.2 | 4.94502277 | −0.05202979 | 2.03E−21 | 0.6356191 | 0 | 0.78508 |
| 74. | TRIM64 | XM_061890.11 | 4.943161345 | −0.17396551 | 2.26E−23 | 0.056348789 | 0 | 0.15062 |
| 75. | LOC402207 | XM_377884.2 | 4.902732221 | −0.01834074 | 6.85E−23 | 0.840730236 | 0 | 0.91594 |
| 76. | LOC729700 | XM_001131081.1 | 4.817202768 | −0.18404267 | 1.04E−23 | 0.033176571 | 0 | 0.10016 |
| 77. | LOC645558 | XM_928577.2 | 4.802893457 | −0.04868182 | 1.18E−22 | 0.597603188 | 0 | 0.75758 |
| 78. | LOC642219 | XM_936370.2 | 4.798732171 | 4.39E−05 | 2.95E−20 | 0.999712853 | 0 | 0.99985 |
| 79. | PRAMEF20 | NM_001099852.1 | 4.795165678 | −0.00363174 | 1.03E−23 | 0.964219718 | 0 | 0.98388 |
| 80. | HBA1 | NM_000558.3 | 4.786546251 | 0.080655575 | 5.55E−23 | 0.365066949 | 0 | 0.56113 |
| 81. | TRIM53 | XR_041244.1 | 4.777537744 | 0.079134921 | 1.16E−22 | 0.390798501 | 0 | 0.58576 |
| 82. | LOC399940 | NM_001136118.1 | 4.726731116 | −0.03723776 | 6.54E−22 | 0.70656513 | 0 | 0.83286 |
| 83. | HBA2 | NM_000517.3 | 4.720819569 | 0.093332131 | 4.76E−24 | 0.231860599 | 0 | 0.41599 |
| 84. | LOC646103 | XM_377879.3 | 4.658033426 | −0.13870545 | 6.41E−21 | 0.214084076 | 0 | 0.39391 |
| 85. | LOC732393 | XR_015873.1 | 4.637178107 | 0.036830325 | 1.36E−21 | 0.714721227 | 0 | 0.8384 |
| 86. | LOC100133446 | XM_001717965.1 | 4.634628768 | 0.116130356 | 4.84E−23 | 0.180518364 | 0 | 0.3498 |
| 87. | LOC100131539 | XM_001724873.1 | 4.629058602 | 0.065239114 | 6.67E−21 | 0.551165724 | 0 | 0.72228 |
| 88. | C12orf50 | NM_152589.1 | 4.521768101 | −0.0213687 | 6.50E−23 | 0.799126907 | 0 | 0.89218 |
| 89. | OR2T34 | NM_001001821.1 | 4.519029057 | −0.05407982 | 5.05E−23 | 0.515865558 | 0 | 0.69307 |
| 90. | TPRX1 | NM_198479.2 | 4.483209754 | −0.0501894 | 1.10E−23 | 0.51112799 | 0 | 0.68941 |
| 91. | LOC402199 | XM_377875.2 | 4.392490269 | 0.081696113 | 3.01E−21 | 0.414457022 | 0 | 0.60764 |
| 92. | LOC646066 | XM_116384.2 | 4.39124129 | −0.02686057 | 2.75E−21 | 0.785823472 | 0 | 0.88355 |
| 93. | ART3 | NM_001179.3 | 4.363323034 | 0.090732461 | 2.34E−22 | 0.300700573 | 0 | 0.49596 |
| 94. | RFPL4A | XM_001719234.1 | 4.347531657 | −0.09031651 | 6.99E−22 | 0.327678483 | 0 | 0.52442 |
| 95. | LOC401860 | XM_377445.3 | 4.272236536 | −0.13415173 | 3.19E−21 | 0.176085184 | 0 | 0.34414 |
| 96. | NXF1 | NM_006362.4 | 4.233044352 | −0.34555942 | 3.92E−22 | 0.000626159 | 0 | 0.00389 |
| 97. | LOC729706 | XM_001131091.1 | 4.227191316 | −0.01625531 | 1.26E−21 | 0.858756767 | 0 | 0.92628 |
| 98. | PRAMEF17 | XM_938420.2 | 4.223085794 | 0.10076379 | 5.13E−20 | 0.366524268 | 0 | 0.56217 |
| 99. | SFRS2B | NM_032102.2 | 4.2153031 | −0.33354085 | 3.27E−22 | 0.000767866 | 0 | 0.00463 |
| 100. | RN5S9 | NR_023371.1 | 4.191231411 | 0.761555557 | 9.29E−23 | 5.90E−09 | 0 | 0 |
| 101. | PPP2R2B | NM_181677.1 | 4.130027648 | −0.10914388 | 1.09E−21 | 0.226560435 | 0 | 0.40935 |
| 102. | ZNF217 | NM_006526.2 | 4.113561121 | −0.32621866 | 6.85E−22 | 0.001060723 | 0 | 0.00605 |
| 103. | ENTPD8 | NM_001033113.1 | 4.072927263 | 0.035234049 | 1.36E−21 | 0.690580498 | 0 | 0.82277 |
| 104. | LOC647827 | XR_018213.1 | 4.053399058 | −0.05208092 | 4.92E−20 | 0.623450204 | 0 | 0.777 |
| 105. | THOC4 | XM_001134346.1 | 4.034801354 | −0.18956718 | 7.79E−22 | 0.035528484 | 0 | 0.10552 |
| 106. | LOC729694 | XM_001131061.1 | 4.028728476 | −0.16550591 | 2.38E−19 | 0.157362785 | 0 | 0.31828 |
| 107. | LOC440053 | NM_001039615.1 | 3.918817013 | −0.09981524 | 3.01E−21 | 0.267252236 | 0 | 0.45861 |
| 108. | LOC440041 | XR_018122.2 | 3.89480714 | 0.021004094 | 2.00E−20 | 0.82868932 | 0 | 0.90915 |
| 109. | HBEGF | NM_001945.1 | 3.868092908 | −0.07673133 | 1.21E−20 | 0.417643656 | 0 | 0.61029 |
| 110. | NEUROG2 | NM_024019.2 | 3.85681225 | −0.00447299 | 1.25E−21 | 0.957178138 | 0 | 0.98027 |
| 111. | PANX2 | NM_052839.2 | 3.830560921 | 0.200055177 | 8.49E−21 | 0.03839939 | 0 | 0.11233 |
| 112. | ZNF280A | NM_080740.3 | 3.797765728 | 0.058889453 | 4.98E−21 | 0.506083159 | 0 | 0.68592 |
| 113. | LOC647366 | XR_018122.1 | 3.783001088 | −0.00333941 | 1.25E−21 | 0.967398855 | 0 | 0.98544 |
| 114. | LOC285697 | XM_210642.1 | 3.782783592 | −0.06561338 | 2.08E−19 | 0.539118315 | 0 | 0.71238 |
| 115. | LOC441081 | XR_017029.1 | 3.763514442 | 0.060234864 | 6.90E−20 | 0.548451457 | 0 | 0.71998 |
| 116. | LOC342933 | XM_938208.2 | 3.75200974 | −0.10207234 | 4.43E−21 | 0.246101014 | 0 | 0.43311 |
| 117. | EGR1 | NM_001964.2 | 3.748663671 | 0.026507346 | 1.16E−21 | 0.7427747 | 0 | 0.85686 |
| 118. | DYNC2H1 | NM_001080463.1 | 3.721145052 | 0.045575817 | 2.77E−21 | 0.587582811 | 0 | 0.74952 |
| 119. | LOC100128202 | XM_001723719.1 | 3.711047498 | −0.11569545 | 8.83E−20 | 0.254283291 | 0 | 0.44265 |
| 120. | PRAMEF8 | NM_001012276.1 | 3.701296487 | 0.166197201 | 1.17E−19 | 0.111383935 | 0 | 0.24918 |
| 121. | SIAH1 | NM_001006610.1 | 3.699147241 | −0.07930283 | 9.21E−22 | 0.319519563 | 0 | 0.51575 |
| 122. | FLJ45337 | NM_207465.1 | 3.685113625 | −0.24611487 | 1.92E−21 | 0.006220997 | 0 | 0.02649 |
| 123. | HSPA2 | NM_021979.3 | 3.669897207 | −0.25459401 | 5.26E−20 | 0.014415927 | 0 | 0.05203 |
| 124. | ODC1 | NM_002539.1 | 3.657122126 | −0.2007912 | 1.71E−21 | 0.019930626 | 0 | 0.06739 |
| 125. | LOC730167 | XM_001726158.1 | 3.655888029 | 1.256728305 | 1.22E−19 | 6.08E−11 | 0 | 0 |
| 126. | FAM90A1 | NM_018088.3 | 3.636367832 | 0.029370652 | 6.18E−19 | 0.786076088 | 0 | 0.88361 |
| 127. | LOC653194 | XM_926449.1 | 3.632643404 | −0.01762031 | 2.57E−18 | 0.879603109 | 0 | 0.93741 |
| 128. | PNMA6B | XM_001721351.1 | 3.603031443 | −0.2188711 | 2.19E−18 | 0.067120975 | 0 | 0.17186 |
| 129. | LOC100132564 | XM_001713808.1 | 3.588075831 | 0.401019931 | 1.43E−18 | 0.001646443 | 0 | 0.00875 |
| 130. | PRR4 | NM_001098538.1 | 3.562468474 | 0.160622534 | 7.87E−22 | 0.042850318 | 0 | 0.12235 |
| 131. | LOC653978 | XM_937424.1 | 3.553895481 | 0.192785536 | 1.85E−19 | 0.063485858 | 0 | 0.16471 |
| 132. | HSPA1A | NM_005345.4 | 3.540028117 | −0.02035008 | 2.76E−18 | 0.858054237 | 0 | 0.92574 |
| 133. | LOC729698 | XM_001131072.1 | 3.538584507 | −0.06572337 | 1.83E−19 | 0.508304015 | 0 | 0.68753 |
| 134. | ZNHIT6 | NM_017953.2 | 3.518983721 | 0.326535866 | 2.16E−21 | 0.000424269 | 0 | 0.00281 |
| 135. | NT5C1B | NM_001002006.1 | 3.495396607 | −0.03655421 | 1.38E−19 | 0.704367331 | 0 | 0.83172 |
| 136. | HNRNPCL1 | NM_001013631.1 | 3.484393042 | −0.10353966 | 2.55E−19 | 0.302153912 | 0 | 0.49734 |
| 137. | CTGLF7 | XM_001714786.1 | 3.436721543 | −0.29494408 | 3.42E−21 | 0.001095911 | 0 | 0.00621 |
| 138. | HSPA1B | NM_005346.3 | 3.401785142 | 0.358980336 | 4.90E−19 | 0.001676423 | 0 | 0.00889 |
| 139. | SLC2A3 | NM_006931.1 | 3.385403683 | 0.101237382 | 2.04E−20 | 0.238786567 | 0 | 0.42467 |
| 140. | DBR1 | NM_016216.2 | 3.378534844 | 0.224692341 | 7.63E−19 | 0.036759428 | 0 | 0.10848 |
| 141. | KLHL15 | NM_030624.1 | 3.37342888 | 0.774243223 | 9.49E−22 | 8.73E−10 | 0 | 0 |
| 142. | LOC650167 | XM_939249.1 | 3.363320722 | 0.048105363 | 7.07E−21 | 0.546500283 | 0 | 0.7183 |
| 143. | LOC100130652 | XM_001719052.1 | 3.330216691 | 0.105014512 | 2.71E−19 | 0.275804678 | 0 | 0.46865 |
| 144. | SPTY2D1 | NM_194285.2 | 3.286224106 | −0.2916034 | 4.47E−21 | 0.000918981 | 0 | 0.00538 |
| 145. | SDHALP1 | NR_003264.1 | 3.276271252 | 0.122529763 | 1.06E−20 | 0.132047058 | 0 | 0.28079 |
| 146. | FBXO33 | NM_203301.1 | 3.219913773 | 0.216167119 | 1.72E−19 | 0.02415313 | 0 | 0.07857 |
| 147. | GTF2F1 | NM_002096.1 | 3.207918515 | 0.290008826 | 7.38E−21 | 0.000980706 | 0 | 0.00568 |
| 148. | FAM90A7 | NM_001136572.1 | 3.19857361 | −0.21371846 | 4.16E−17 | 0.082629899 | 0 | 0.20087 |
| 149. | TFIP11 | NM_012143.2 | 3.191115229 | 0.112444499 | 5.78E−21 | 0.142573457 | 0 | 0.29687 |
| 150. | PRAMEF14 | NM_001099854.1 | 3.185645753 | −0.16938858 | 1.23E−17 | 0.136924741 | 0 | 0.28794 |
| 151. | JUP | NM_002230.1 | 3.172493972 | −0.10574784 | 8.62E−19 | 0.277847276 | 0 | 0.47091 |
| 152. | RAB6B | NM_016577.3 | 3.170174833 | 0.007069344 | 1.31E−17 | 0.948962003 | 0 | 0.97536 |
| 153. | CLDN14 | NM_012130.2 | 3.147780532 | −0.3005501 | 1.06E−20 | 0.000705891 | 0 | 0.00431 |
| 154. | LOC653111 | XM_926073.2 | 3.139215982 | −0.09966257 | 5.28E−18 | 0.345009515 | 0 | 0.54173 |
| 155. | FGFR3 | NM_022965.1 | 3.111910187 | 0.041474475 | 7.15E−20 | 0.617422726 | 0 | 0.77246 |
| 156. | LOC642446 | XM_001717781.1 | 3.10134434 | 0.187941596 | 2.37E−18 | 0.068762031 | 0 | 0.17497 |
| 157. | LOC649330 | XM_001723218.1 | 3.10126304 | −0.26972993 | 1.95E−17 | 0.022495581 | 0 | 0.07403 |
| 158. | SOX9 | NM_000346.2 | 3.099073039 | −0.30863513 | 1.41E−20 | 0.000545661 | 0 | 0.00347 |
| 159. | KBTBD8 | NM_032505.1 | 3.075609437 | 0.010765435 | 5.01E−18 | 0.915890843 | 0 | 0.95951 |
| 160. | LOC727828 | XR_015137.1 | 3.053539854 | −0.00555958 | 5.39E−20 | 0.944623917 | 0 | 0.97324 |
| 161. | PPP1R14C | NM_030949.2 | 3.048726581 | −0.01369375 | 8.26E−18 | 0.894960188 | 0 | 0.94666 |
| 162. | LOC729724 | XM_001131132.1 | 3.038453461 | 0.111242726 | 8.02E−20 | 0.18128368 | 0 | 0.35074 |
| 163. | LOC652433 | XM_941875.1 | 3.022406734 | −0.03197171 | 1.82E−17 | 0.765570805 | 0 | 0.87176 |
| 164. | LOC391761 | XM_373073.2 | 3.009217657 | −0.05893793 | 4.67E−18 | 0.554866398 | 0 | 0.7253 |
| 165. | IFRD1 | NM_001550.2 | 2.990137587 | −0.19198734 | 9.17E−19 | 0.044831594 | 0 | 0.12646 |
| 166. | LOC342934 | XM_292724.5 | 2.987840631 | −0.06004995 | 1.28E−16 | 0.609711079 | 0 | 0.7666 |
| 167. | DBNDD2 | NM_001048223.1 | 2.986531035 | 0.225696775 | 1.48E−19 | 0.011894374 | 0 | 0.04467 |
| 168. | MGC61598 | XM_939432.1 | 2.969400698 | 0.095811933 | 5.41E−19 | 0.281754516 | 0 | 0.47532 |
| 169. | CSE1L | NM_177436.1 | 2.936775247 | 0.072869191 | 3.16E−19 | 0.392112884 | 0 | 0.58683 |
| 170. | NEFM | NM_005382.1 | 2.92509738 | 0.848466864 | 8.17E−18 | 4.02E−08 | 0 | 0 |
| 171. | LOC650236 | XR_036872.1 | 2.915370575 | −0.05944742 | 1.48E−17 | 0.562576956 | 0 | 0.73099 |
| 172. | LOC100130311 | XM_001724111.1 | 2.912233006 | 0.143640536 | 7.08E−19 | 0.111098741 | 0 | 0.24867 |
| 173. | EOMES | NM_005442.2 | 2.897434573 | 0.064838479 | 8.70E−20 | 0.409051786 | 0 | 0.60273 |
| 174. | LOC645373 | XM_928412.1 | 2.89149778 | −0.06601509 | 1.86E−16 | 0.569921908 | 0 | 0.73685 |
| 175. | PELI1 | NM_020651.2 | 2.885705123 | 0.148281618 | 3.32E−18 | 0.124626886 | 0 | 0.26982 |
| 176. | LOC285299 | XM_936463.2 | 2.839952293 | −0.03457605 | 4.43E−17 | 0.743286409 | 0 | 0.85714 |
| 177. | LOC652349 | XM_941777.1 | 2.835445356 | 0.016964784 | 6.30E−17 | 0.87431718 | 0 | 0.93496 |
| 178. | LOC400464 | XR_041115.1 | 2.831055937 | −0.07853086 | 2.32E−18 | 0.388228712 | 0 | 0.58348 |
| 179. | LOC391747 | XM_373059.2 | 2.788628167 | −0.10487233 | 8.75E−20 | 0.172080374 | 0 | 0.33855 |
| 180. | BAMBI | NM_012342.2 | 2.783131683 | 0.110053957 | 6.13E−19 | 0.193134544 | 0 | 0.36739 |
| 181. | PELI2 | NM_021255.2 | 2.782423319 | −0.37316879 | 9.34E−18 | 0.000780669 | 0 | 0.00469 |
| 182. | T1560 | NM_199048.1 | 2.769356823 | 0.02012096 | 1.01E−16 | 0.851350752 | 0 | 0.92166 |
| 183. | KCNH4 | NM_012285.1 | 2.748842971 | −0.07365183 | 8.94E−17 | 0.489289998 | 0 | 0.67248 |
| 184. | AMACR | NM_014324.4 | 2.743048821 | 0.084305604 | 4.20E−18 | 0.35465387 | 0 | 0.55115 |
| 185. | SLC3A1 | NM_000341.2 | 2.73626768 | 0.003792308 | 7.66E−19 | 0.963231713 | 0 | 0.98339 |
| 186. | DYNLL2 | NM_080677.1 | 2.735918959 | 0.652116243 | 2.47E−17 | 1.75E−06 | 0 | 3.00E−05 |
| 187. | LOC642843 | XM_926241.2 | 2.72784969 | −0.0584813 | 7.41E−17 | 0.575540743 | 0 | 0.74024 |
| 188. | LOC100129053 | XM_001718702.1 | 2.724096232 | 0.0995082 | 1.76E−17 | 0.308376623 | 0 | 0.50373 |
| 189. | CCNJ | NM_019084.2 | 2.694767424 | 0.079155108 | 2.77E−18 | 0.365798327 | 0 | 0.56146 |
| 190. | BZW2 | NM_014038.1 | 2.677197026 | −0.4002905 | 5.61E−19 | 5.53E−05 | 0 | 5.00E−04 |
| 191. | CWC15 | NM_016403.3 | 2.643111234 | 0.09338116 | 3.88E−18 | 0.287130937 | 0 | 0.48154 |
| 192. | CD24 | NM_013230.2 | 2.623896274 | −0.35857296 | 9.32E−18 | 0.000655138 | 0 | 0.00405 |
| 193. | C9orf61 | NM_004816.2 | 2.614048685 | −0.256295 | 1.47E−16 | 0.020853562 | 0 | 0.06978 |
| 194. | DENND2C | NM_198459.2 | 2.608835297 | −0.26836909 | 6.99E−18 | 0.005865841 | 0 | 0.02523 |
| 195. | ARS2 | NM_015908.4 | 2.602017661 | −0.20020565 | 5.95E−18 | 0.03056309 | 0 | 0.09388 |
| 196. | YRDC | NM_024640.3 | 2.600956126 | 0.000579047 | 1.56E−18 | 0.994305939 | 0 | 0.99763 |
| 197. | USP29 | NM_020903.2 | 2.596878397 | −0.09045716 | 3.88E−18 | 0.293726512 | 0 | 0.48842 |
| 198. | EYA3 | NM_001990.2 | 2.572156831 | −0.016718 | 8.10E−18 | 0.848369803 | 0 | 0.91967 |
| 199. | LOC646914 | XM_929877.1 | 2.569191334 | −0.02473384 | 9.21E−18 | 0.778586131 | 0 | 0.87953 |
| 200. | PABPN1 | NM_004643.1 | 2.567302816 | −0.25853738 | 3.01E−18 | 0.005006723 | 0 | 0.02213 |
| 201. | MGC40489 | XR_016048.1 | 2.541736559 | −0.46037932 | 1.56E−16 | 0.000170117 | 0 | 0.00129 |
| 202. | Cllorf82 | NM_145018.2 | 2.530678429 | −0.27379577 | 2.31E−17 | 0.006360255 | 0 | 0.02696 |
| 203. | C14orf102 | NM_017970.2 | 2.529377841 | 0.176891759 | 4.39E−18 | 0.044016052 | 0 | 0.12468 |
| 204. | FAM107B | NM_031453.2 | 2.529113421 | −0.18414043 | 7.44E−17 | 0.067746068 | 0 | 0.17309 |
| 205. | CYCSL1 | NR_001561.1 | 2.505598739 | 0.37325739 | 2.32E−18 | 0.000131642 | 0 | 0.00104 |
| 206. | DEFB103B | NM_018661.3 | 2.499063846 | −0.03506737 | 1.60E−15 | 0.754511987 | 0 | 0.86535 |
| 207. | LOC646508 | XM_937570.1 | 2.498284454 | −0.04201402 | 4.66E−17 | 0.652242402 | 0 | 0.79692 |
| 208. | CSRNP3 | NM_024969.2 | 2.495907791 | −0.07940541 | 2.30E−16 | 0.435421955 | 0 | 0.62628 |
| 209. | CXADR | NM_001338.3 | 2.494457445 | −0.07004782 | 6.78E−16 | 0.514379205 | 0 | 0.69223 |
| 210. | C13orf34 | NM_024808.2 | 2.49194451 | −0.00212698 | 3.53E−18 | 0.979075125 | 0 | 0.99072 |
| 211. | LOC100134322 | XR_037416.1 | 2.488088458 | 0.19623414 | 1.37E−17 | 0.033397294 | 0 | 0.10071 |
| 212. | PRAMEF21 | NM_001100114.1 | 2.487018924 | −0.06826812 | 3.03E−17 | 0.454028195 | 0 | 0.64319 |
| 213. | LOC728450 | XM_001131473.2 | 2.474009279 | 0.134655982 | 1.20E−17 | 0.127894561 | 0 | 0.27504 |
| 214. | SFRS17A | NM_005088.2 | 2.456301079 | 0.240381708 | 3.93E−17 | 0.014336264 | 0 | 0.05183 |
| 215. | FLJ45139 | NM_001001692.1 | 2.451388314 | 0.100850114 | 8.41E−16 | 0.347811335 | 0 | 0.54453 |
| 216. | C6orf117 | NM_138409.1 | 2.446043441 | −0.06998426 | 1.53E−17 | 0.419253401 | 0 | 0.61133 |
| 217. | NOLC1 | NM_004741.1 | 2.438536354 | 0.49765378 | 1.73E−17 | 1.09E−05 | 0 | 0.00013 |
| 218. | SYNJ1 | NM_203446.1 | 2.433164214 | −0.07514634 | 2.72E−16 | 0.452617734 | 0 | 0.64184 |
| 219. | MGC10997 | NR_001565.1 | 2.426009364 | −0.29360506 | 2.86E−18 | 0.001131964 | 0 | 0.00638 |
| 220. | LOC649563 | XM_938635.2 | 2.424763127 | −0.0611379 | 1.45E−16 | 0.525444143 | 0 | 0.70146 |
| 221. | KPNA2 | NM_002266.2 | 2.416323657 | −0.50749956 | 2.90E−16 | 4.68E−05 | 0 | 0.00044 |
| 222. | MIR2278 | NR_031755.1 | 2.412240829 | 0.097675867 | 2.53E−15 | 0.382579115 | 0 | 0.57813 |
| 223. | ZNF622 | NM_033414.2 | 2.40621318 | 0.014027344 | 6.58E−18 | 0.862356543 | 0 | 0.92843 |
| 224. | CTR9 | NM_014633.3 | 2.402374134 | −0.02160461 | 1.54E−17 | 0.798169527 | 0 | 0.89173 |
| 225. | NCRNA00092 | NR_024129.1 | 2.394551708 | −0.11225744 | 3.98E−16 | 0.267969634 | 0 | 0.45928 |
| 226. | FAM46C | NM_017709.3 | 2.389165515 | −0.12894575 | 5.82E−14 | 0.325974529 | 0 | 0.52277 |
| 227. | SLC2A14 | NM_153449.2 | 2.374348717 | 0.247150911 | 5.24E−16 | 0.021643048 | 0 | 0.07182 |
| 228. | PRRG4 | NM_024081.4 | 2.369418474 | 0.209621361 | 8.34E−16 | 0.051930043 | 0 | 0.14159 |
| 229. | SLIT2 | NM_004787.1 | 2.368717174 | 0.850098551 | 2.92E−17 | 3.76E−09 | 0 | 0 |
| 230. | CRY1 | NM_004075.2 | 2.341679414 | −0.12675954 | 1.85E−17 | 0.13815092 | 0 | 0.28994 |
| 231. | ID2 | NM_002166.4 | 2.335808841 | 0.275613793 | 3.13E−15 | 0.018296862 | 0 | 0.06306 |
| 232. | PRAMEF19 | NM_001099790.1 | 2.335767802 | −0.03732366 | 5.68E−18 | 0.632899653 | 0 | 0.78322 |
| 233. | NFYA | NM_002505.3 | 2.334723566 | 0.915312276 | 4.47E−16 | 9.21E−09 | 0 | 0 |
| 234. | LOC732416 | XM_001133386.1 | 2.328833136 | 0.05578418 | 4.22E−17 | 0.519939778 | 0 | 0.69684 |
| 235. | NUP50 | NM_007172.3 | 2.323544688 | 1.034765786 | 1.70E−17 | 5.78E−11 | 0 | 0 |
| 236. | LOC645137 | XM_928167.2 | 2.322677192 | −0.15240225 | 7.82E−17 | 0.097906179 | 0 | 0.22652 |
| 237. | LOC651709 | XM_001732813.1 | 2.304254222 | 0.094448257 | 2.01E−17 | 0.257839464 | 0 | 0.44705 |
| 238. | TMEM185A | NM_032508.1 | 2.292123236 | 0.232711608 | 2.08E−17 | 0.009348785 | 0 | 0.03688 |
| 239. | LOC648533 | XM_937587.1 | 2.283913086 | 0.037364122 | 5.74E−13 | 0.790074552 | 0 | 0.88629 |
| 240. | WDR47 | NM_014969.4 | 2.276954944 | 0.140077923 | 4.04E−16 | 0.150823161 | 0 | 0.30902 |
| 241. | RMRP | NR_003051.2 | 2.266910293 | 0.073341561 | 1.81E−15 | 0.476105118 | 0 | 0.66141 |
| 242. | C8orf33 | NM_023080.1 | 2.250696465 | −0.06074557 | 5.76E−17 | 0.476217777 | 0 | 0.66141 |
| 243. | DUSP12 | NM_007240.1 | 2.249346009 | 0.098691596 | 6.04E−18 | 0.199416906 | 0 | 0.37517 |
| 244. | FAM90A9 | XM_496956.4 | 2.247726801 | 0.016928845 | 1.30E−16 | 0.847999834 | 0 | 0.91942 |
| 245. | ARIH1 | NM_005744.2 | 2.24300359 | 0.783967696 | 3.40E−17 | 6.64E−09 | 0 | 0 |
| 246. | TRIM23 | NM_001656.3 | 2.234876163 | 0.630256517 | 7.41E−18 | 5.77E−08 | 0 | 0 |
| 247. | ADPGK | NM_031284.3 | 2.232580766 | 0.091955001 | 2.16E−17 | 0.257304543 | 0 | 0.44637 |
| 248. | PVRL3 | NM_015480.1 | 2.223358786 | 0.447298551 | 1.67E−15 | 0.000209677 | 0 | 0.00154 |
| 249. | ZNF214 | NM_013249.1 | 2.219239817 | 0.16599169 | 2.09E−14 | 0.156088036 | 0 | 0.31667 |
| 250. | HSPH1 | NM_006644.2 | 2.216889189 | −0.48791765 | 1.07E−17 | 2.89E−06 | 0 | 4.00E−05 |
| 251. | PIM1 | NM_002648.2 | 2.214932099 | −0.67564311 | 4.69E−18 | 1.13E−08 | 0 | 0 |
| 252. | PSPN | NM_004158.2 | 2.202671331 | −0.0387554 | 1.84E−15 | 0.697279542 | 0 | 0.82706 |
| 253. | HOXB2 | NM_002145.3 | 2.202149933 | −0.04068855 | 9.93E−17 | 0.634244204 | 0 | 0.7842 |
| 254. | LOC100133588 | XM_001714755.1 | 2.200369303 | 0.039776542 | 5.95E−14 | 0.739658675 | 0 | 0.85497 |
| 255. | C1orf63 | NM_020317.3 | 2.189819772 | −0.13903881 | 2.44E−12 | 0.344977916 | 0 | 0.54172 |
| 256. | STK3 | NM_006281.2 | 2.185603351 | −0.21498857 | 3.10E−15 | 0.044513409 | 0 | 0.12581 |
| 257. | HEY1 | NM_001040708.1 | 2.180101337 | −0.04270689 | 1.45E−16 | 0.621081524 | 0 | 0.77541 |
| 258. | LOC728429 | XR_038921.1 | 2.177569373 | −0.02833864 | 2.42E−15 | 0.776523643 | 0 | 0.87813 |
| 259. | HNRPDL | NR_003249.1 | 2.175352802 | 0.016888999 | 1.34E−16 | 0.843620455 | 0 | 0.91755 |
| 260. | LOC727846 | XM_001126140.1 | 2.175344255 | 0.003661846 | 2.12E−15 | 0.970479736 | 0 | 0.98654 |
| 261. | LOC391045 | XM_372780.3 | 2.165905106 | 0.353079083 | 2.62E−14 | 0.004826112 | 0 | 0.02149 |
| 262. | UBL3 | NM_007106.2 | 2.164486474 | −0.80700031 | 8.07E−17 | 4.82E−09 | 0 | 0 |
| 263. | ZSCAN2 | NM_017894.4 | 2.151267294 | −0.19990085 | 8.39E−17 | 0.023707117 | 0 | 0.07732 |
| 264. | PNO1 | NM_020143.2 | 2.150848548 | 0.248468101 | 2.17E−17 | 0.003888212 | 0 | 0.018 |
| 265. | GPR37 | NM_005302.2 | 2.121339069 | 0.215068748 | 1.36E−14 | 0.054464486 | 0 | 0.14697 |
| 266. | TSPAN13 | NM_014399.3 | 2.116834376 | −0.14687037 | 1.64E−15 | 0.133289273 | 0 | 0.28287 |
| 267. | SNIP1 | NM_024700.2 | 2.104097443 | −0.17404984 | 4.31E−17 | 0.03536112 | 0 | 0.10521 |
| 268. | MED26 | NM_004831.3 | 2.099784167 | 0.137079716 | 5.67E−16 | 0.135137322 | 0 | 0.28545 |
| 269. | C6orf191 | NM_001010876.1 | 2.090649638 | −0.05752287 | 1.93E−15 | 0.545204643 | 0 | 0.71727 |
| 270. | LOC645381 | XR_038557.1 | 2.089101452 | −0.1265825 | 1.39E−16 | 0.135422085 | 0 | 0.28587 |
| 271. | PPP1R15A | NM_014330.2 | 2.079608755 | 0.566757113 | 1.80E−16 | 1.15E−06 | 0 | 2.00E−05 |
| 272. | RRN3 | NM_018427.3 | 2.068096238 | 0.107184797 | 2.56E−16 | 0.212066606 | 0 | 0.39151 |
| 273. | CBARA1 | NM_006077.2 | 2.064175867 | −0.15426321 | 2.91E−15 | 0.117698621 | 0 | 0.25949 |
| 274. | NGDN | NM_015514.1 | 2.057504656 | 0.408772415 | 1.43E−15 | 0.000219075 | 0 | 0.0016 |
| 275. | MED31 | NM_016060.2 | 2.054924581 | 0.31225842 | 3.76E−16 | 0.001432204 | 0 | 0.00778 |
| 276. | SON | NM_032195.1 | 2.054844468 | −0.28384608 | 5.39E−15 | 0.008436028 | 0 | 0.03398 |
| 277. | STX6 | NM_005819.4 | 2.053005326 | −0.46956758 | 5.95E−15 | 0.00010875 | 0 | 0.00088 |
| 278. | C1orf55 | NM_152608.3 | 2.049481602 | −0.36315178 | 8.73E−15 | 0.001592623 | 0 | 0.00851 |
| 279. | SGK | NM_005627.2 | 2.039844812 | −0.11041486 | 4.65E−16 | 0.206764605 | 0 | 0.38453 |
| 280. | RPPH1 | NR_002312.1 | 2.037174446 | 0.226914242 | 2.02E−15 | 0.022037966 | 0 | 0.07279 |
| 281. | CEP78 | NM_032171.1 | 2.035493751 | −0.13956271 | 1.64E−16 | 0.096278858 | 0 | 0.22372 |
| 282. | CASP6 | NM_032992.2 | 2.035084058 | 0.233567145 | 3.68E−15 | 0.02223849 | 0 | 0.07334 |
| 283. | ARID3B | NM_006465.2 | 2.024103942 | −0.0745492 | 2.94E−16 | 0.373855818 | 0 | 0.56922 |
| 284. | AVPI1 | NM_021732.1 | 2.021560017 | 0.188378801 | 9.68E−17 | 0.024283729 | 0 | 0.07885 |
| 285. | RNGTT | NM_003800.3 | 2.017732238 | −0.59159318 | 4.94E−17 | 1.45E−07 | 0 | 0 |
| 286. | KIAA0020 | NM_014878.4 | 2.017163694 | 0.017829862 | 1.80E−15 | 0.844688542 | 0 | 0.91796 |
| 287. | SLC25A44 | NM_014655.1 | 2.016768709 | −0.0765425 | 1.49E−16 | 0.343019748 | 0 | 0.5399 |
| 288. | RBM12 | NM_006047.4 | 2.012922532 | −0.37476245 | 1.03E−16 | 9.94E−05 | 0 | 0.00082 |
| 289. | CXCR4 | NM_001008540.1 | 2.003654471 | −0.05210245 | 2.10E−14 | 0.613887002 | 0 | 0.76996 |
| 290. | PDSS1 | NM_014317.3 | 2.002705196 | −0.07459033 | 1.33E−15 | 0.405603148 | 0 | 0.59918 |
| 291. | ISOC1 | NM_016048.1 | 2.002585299 | −0.61873447 | 7.89E−16 | 5.78E−07 | 0 | 1.00E−05 |
| 292. | SERTAD1 | NM_013376.3 | 2.001534685 | 0.410030021 | 1.32E−14 | 0.000524692 | 0 | 0.00336 |
| 293. | CCDC58 | NM_001017928.2 | 2.001365764 | 0.076783734 | 3.92E−15 | 0.418170823 | 0 | 0.6105 |
| 294. | DNAJC25 | NM_001015882.2 | 1.995453072 | −0.50719957 | 5.50E−17 | 1.28E−06 | 0 | 2.00E−05 |
| 295. | LSG1 | NM_018385.1 | 1.994244729 | −0.06468359 | 7.42E−16 | 0.454331188 | 0 | 0.64342 |
| 296. | HSPA6 | NM_002155.3 | 1.993905834 | 0.128022925 | 1.90E−16 | 0.120483692 | 0 | 0.26369 |
| 297. | FRG2B | NM_001080998.1 | 1.993396796 | 0.13147002 | 1.21E−15 | 0.146280541 | 0 | 0.30263 |
| 298. | CD9 | NM_001769.2 | 1.991567881 | −0.00478773 | 2.64E−14 | 0.96313223 | 0 | 0.98338 |
| 299. | LOC652080 | XM_941404.1 | 1.987117494 | 0.173413351 | 8.20E−15 | 0.085827376 | 0 | 0.20642 |
| 300. | RAB11FIP1 | NM_001002814.1 | 1.98677521 | −0.47650768 | 2.06E−16 | 7.22E−06 | 0 | 9.00E−05 |
| 301. | RBBP6 | NM_032626.5 | 1.98571063 | −0.1339181 | 2.18E−13 | 0.255048947 | 0 | 0.4434 |
| 302. | INO80C | NM_194281.3 | 1.979828473 | −0.11275614 | 3.37E−14 | 0.286888863 | 0 | 0.48127 |
| 303. | TRA2A | NM_013293.3 | 1.977977513 | −0.10947385 | 1.54E−15 | 0.224834701 | 0 | 0.40702 |
| 304. | LYAR | NM_017816.1 | 1.97693312 | 0.000874971 | 1.25E−15 | 0.992016109 | 0 | 0.99687 |
| 305. | C1QTNF3 | NM_181435.4 | 1.974262994 | −0.02264422 | 1.81E−16 | 0.774302617 | 0 | 0.87667 |
| 306. | KLC1 | NM_005552.4 | 1.96683651 | −0.06609191 | 1.44E−15 | 0.454063934 | 0 | 0.6432 |
| 307. | LOC399988 | XR_018287.2 | 1.965863889 | −0.41310115 | 2.86E−15 | 0.000178798 | 0 | 0.00134 |
| 308. | MED10 | NM_032286.2 | 1.965185968 | 0.599425454 | 1.80E−16 | 2.22E−07 | 0 | 1.00E−05 |
| 309. | LOC391763 | XM_001715080.1 | 1.963119789 | 0.092872676 | 3.74E−13 | 0.434006893 | 0 | 0.62476 |
| 310. | RGS4 | NM_005613.3 | 1.951453189 | −0.48369978 | 1.18E−15 | 1.53E−05 | 0 | 0.00017 |
| 311. | POLR3K | NM_016310.2 | 1.950192615 | 0.058729235 | 5.12E−15 | 0.529572686 | 0 | 0.7048 |
| 312. | OSBPL8 | NM_020841.4 | 1.950078352 | 1.0547593 | 5.57E−15 | 3.91E−10 | 0 | 0 |
| 313. | PNN | NM_002687.3 | 1.949442084 | 0.044126108 | 3.43E−16 | 0.585331055 | 0 | 0.74731 |
| 314. | TUBB2C | NM_006088.5 | 1.948000242 | −0.53914845 | 2.08E−16 | 1.03E−06 | 0 | 2.00E−05 |
| 315. | SNAI1 | NM_005985.2 | 1.943131852 | −0.03657258 | 1.02E−14 | 0.703998321 | 0 | 0.83171 |
| 316. | LOC651390 | XM_942401.1 | 1.937495897 | −0.07745626 | 2.67E−14 | 0.445805611 | 0 | 0.63538 |
| 317. | EXOSC10 | NM_002685.2 | 1.936970335 | 0.277137164 | 1.08E−11 | 0.058545399 | 0 | 0.15506 |
| 318. | GPBAR1 | NM_170699.2 | 1.936378949 | −0.12982997 | 7.31E−15 | 0.177666352 | 0 | 0.34619 |
| 319. | NEFH | NM_021076.2 | 1.936165554 | 0.099517054 | 9.82E−15 | 0.304158408 | 0 | 0.49929 |
| 320. | PEG10 | XM_499343.2 | 1.93541239 | −0.09718045 | 4.86E−13 | 0.413319297 | 0 | 0.60668 |
| 321. | LOC643336 | XM_001718563.1 | 1.933728249 | 1.023196188 | 1.62E−15 | 1.85E−10 | 0 | 0 |
| 322. | HNRPA1L-2 | NR_002944.2 | 1.921260156 | −0.89796096 | 5.13E−16 | 5.51E−10 | 0 | 0 |
| 323. | FRAT2 | NM_012083.2 | 1.918313267 | −0.05118051 | 5.04E−16 | 0.529046462 | 0 | 0.70431 |
| 324. | HSPB3 | NM_006308.1 | 1.913506168 | −0.79338174 | 3.48E−14 | 1.44E−07 | 0 | 0 |
| 325. | CRLF3 | NM_015986.2 | 1.911700836 | −0.14486974 | 2.52E−16 | 0.073921065 | 0 | 0.18466 |
| 326. | MIR503 | NR_030228.1 | 1.909586785 | −0.13610263 | 6.88E−14 | 0.202499117 | 0 | 0.37906 |
| 327. | POLR1B | NM_019014.3 | 1.90405424 | −0.13127473 | 2.09E−15 | 0.140374918 | 0 | 0.29338 |
| 328. | LOC643731 | XM_927019.1 | 1.903360811 | −0.00093141 | 4.70E−13 | 0.993571402 | 0 | 0.99732 |
| 329. | CCDC59 | NM_014167.2 | 1.892481485 | 0.2508832 | 4.34E−15 | 0.010195171 | 0 | 0.03944 |
| 330. | ETNK1 | NM_001039481.1 | 1.89168344 | 0.064440994 | 3.57E−13 | 0.570766866 | 0 | 0.73739 |
| 331. | NOP58 | NM_015934.3 | 1.886662902 | 0.224260185 | 1.13E−15 | 0.012880266 | 0 | 0.04768 |
| 332. | BRIX1 | NM_018321.3 | 1.886222351 | 0.299106046 | 7.02E−15 | 0.003529392 | 0 | 0.01657 |
| 333. | SNRNP70 | NM_003089.4 | 1.884727124 | 0.017757319 | 1.78E−15 | 0.834507764 | 0 | 0.91261 |
| 334. | ELOF1 | NM_032377.3 | 1.884130525 | −0.30080573 | 3.39E−15 | 0.002480132 | 0 | 0.0123 |
| 335. | CCNT2 | NM_058241.1 | 1.876469209 | −0.32206863 | 7.60E−16 | 0.0006697 | 0 | 0.00412 |
| 336. | NANS | NM_018946.2 | 1.871711876 | −0.00660686 | 4.71E−14 | 0.947536181 | 0 | 0.97449 |
| 337. | LOC100129630 | XM_001714940.1 | 1.870936058 | −0.10375837 | 1.63E−14 | 0.281144687 | 0 | 0.47443 |
| 338. | TAF4B | NM_005640.1 | 1.86960695 | −0.04440194 | 6.38E−14 | 0.663918819 | 0 | 0.805 |
| 339. | SRP19 | NM_003135.1 | 1.866226706 | 0.227022607 | 8.39E−15 | 0.020599821 | 0 | 0.06904 |
| 340. | SGCG | NM_000231.1 | 1.863213339 | 0.125027437 | 3.80E−15 | 0.163210943 | 0 | 0.32631 |
| 341. | CCNE1 | NM_057182.1 | 1.863055664 | −0.12768816 | 3.24E−13 | 0.257494264 | 0 | 0.44663 |
| 342. | SLC40A1 | NM_014585.3 | 1.858510401 | −0.42386003 | 3.33E−15 | 7.95E−05 | 0 | 0.00068 |
| 343. | PKIB | NM_032471.4 | 1.858446651 | −0.17186416 | 2.73E−14 | 0.087611639 | 0 | 0.20959 |
| 344. | LARP1B | NM_032239.2 | 1.857923432 | −0.26022457 | 3.16E−16 | 0.002599598 | 0 | 0.01279 |
| 345. | SNORD56 | NR_002739.1 | 1.854937162 | −0.16574948 | 6.20E−14 | 0.112311042 | 0 | 0.25059 |
| 346. | SIRT1 | NM_012238.3 | 1.843963503 | 0.207080634 | 2.73E−13 | 0.068322693 | 0 | 0.1741 |
| 347. | KIAA0114 | NR_024031.1 | 1.840881565 | −0.31600952 | 3.86E−14 | 0.003849075 | 0 | 0.01784 |
| 348. | LOC399937 | XM_374917.3 | 1.840861475 | 0.000791404 | 5.08E−14 | 0.993631669 | 0 | 0.99732 |
| 349. | CLK1 | NM_001024646.1 | 1.840475237 | 0.075199503 | 3.48E−12 | 0.548449186 | 0 | 0.71998 |
| 350. | LOC391092 | XM_372792.2 | 1.837421253 | 0.110663076 | 9.40E−14 | 0.286465176 | 0 | 0.48071 |
| 351. | SEC61A2 | NM_018144.2 | 1.837165462 | 0.209041091 | 3.40E−15 | 0.022761839 | 0 | 0.07478 |
| 352. | KIF21A | NM_017641.2 | 1.836729176 | 0.015294409 | 2.76E−15 | 0.856782313 | 0 | 0.92516 |
| 353. | LOC651816 | XM_941060.1 | 1.836368433 | −0.53879136 | 3.60E−15 | 3.67E−06 | 0 | 5.00E−05 |
| 354. | KCNA1 | NM_000217.2 | 1.831756083 | 0.028418893 | 6.64E−13 | 0.802216761 | 0 | 0.89368 |
| 355. | PPM1B | NM_177968.2 | 1.830564265 | −0.03419808 | 1.49E−14 | 0.71162437 | 0 | 0.83624 |
| 356. | MYLIP | NM_013262.3 | 1.824056957 | −0.29688235 | 2.69E−15 | 0.001923181 | 0 | 0.00997 |
| 357. | KATNA1 | NM_007044.2 | 1.819203659 | 0.113184818 | 6.97E−15 | 0.208410535 | 0 | 0.3867 |
| 358. | MBD2 | NM_015832.3 | 1.817924533 | −0.07679197 | 5.80E−15 | 0.383295862 | 0 | 0.5789 |
| 359. | MED13 | NM_005121.2 | 1.816759236 | 0.026398068 | 1.83E−15 | 0.747828875 | 0 | 0.86101 |
| 360. | SOX4 | NM_003107.2 | 1.808997932 | −0.21735552 | 7.84E−14 | 0.039377791 | 0 | 0.11469 |
| 361. | SERPINI1 | NM_005025.3 | 1.808914219 | 0.035398211 | 1.10E−14 | 0.694040672 | 0 | 0.82487 |
| 362. | LOC389633 | XM_372030.4 | 1.806620634 | −0.08520179 | 1.06E−14 | 0.346747877 | 0 | 0.54351 |
| 363. | CHORDC1 | NM_012124.1 | 1.804721727 | 0.126125821 | 3.39E−15 | 0.144922027 | 0 | 0.30054 |
| 364. | ARC | NM_015193.3 | 1.801587557 | −0.18800477 | 2.20E−15 | 0.031295976 | 0 | 0.09574 |
| 365. | INSM1 | NM_002196.2 | 1.800557044 | 0.230573913 | 1.78E−14 | 0.019367652 | 0 | 0.06592 |
| 366. | RBM39 | NM_184234.1 | 1.799763318 | 0.263046794 | 2.84E−14 | 0.010347921 | 0 | 0.0399 |
| 367. | LOC642538 | XM_926027.2 | 1.792389919 | 0.125787579 | 2.61E−14 | 0.186933675 | 0 | 0.35927 |
| 368. | TESK2 | NM_007170.2 | 1.787330928 | 0.126864846 | 4.92E−15 | 0.146548958 | 0 | 0.30296 |
| 369. | PDRG1 | NM_030815.2 | 1.782963685 | 0.062203847 | 4.00E−14 | 0.514127 | 0 | 0.69201 |
| 370. | KLF17 | NM_173484.3 | 1.778806102 | 0.080447536 | 4.47E−14 | 0.402160621 | 0 | 0.59562 |
| 371. | FAM90A12 | XM_496961.3 | 1.774389657 | 0.048242903 | 9.46E−14 | 0.626471745 | 0 | 0.77881 |
| 372. | LOC388275 | XM_928429.1 | 1.772108431 | −0.93959193 | 1.86E−14 | 1.63E−09 | 0 | 0 |
| 373. | ZNF365 | NM_014951.2 | 1.771160957 | −0.37403373 | 2.58E−14 | 0.000552145 | 0 | 0.00351 |
| 374. | RNF122 | NM_024787.2 | 1.768869814 | 0.191599957 | 3.76E−14 | 0.051887596 | 0 | 0.14151 |
| 375. | KDM5B | NM_006618.3 | 1.762560409 | −0.11116732 | 1.38E−14 | 0.218359857 | 0 | 0.39927 |
| 376. | HOXB6 | NM_018952.4 | 1.761678941 | 0.064514742 | 6.42E−14 | 0.504431835 | 0 | 0.68485 |
| 377. | C21orf91 | NM_017447.2 | 1.759665752 | 0.079481476 | 5.99E−14 | 0.409986098 | 0 | 0.60376 |
| 378. | FAM90A5 | XM_496947.4 | 1.757517326 | 0.104189301 | 1.59E−15 | 0.19548293 | 0 | 0.37021 |
| 379. | FAM133B | NM_152789.2 | 1.756086039 | 0.088582621 | 2.09E−14 | 0.331959546 | 0 | 0.52857 |
| 380. | NIPSNAP3A | NM_015469.1 | 1.750667829 | −0.06938901 | 1.89E−14 | 0.441356857 | 0 | 0.63143 |
| 381. | RNF152 | NM_173557.2 | 1.749157783 | 0.225296538 | 1.87E−13 | 0.035514631 | 0 | 0.10551 |
| 382. | C13orf31 | NM_153218.1 | 1.748039669 | 0.014786819 | 1.38E−14 | 0.866326588 | 0 | 0.93043 |
| 383. | ELOVL4 | NM_022726.2 | 1.746070975 | −0.32373904 | 2.27E−15 | 0.000556816 | 0 | 0.00353 |
| 384. | TP53BP2 | NM_001031685.2 | 1.746064864 | −0.03883125 | 9.55E−15 | 0.652528964 | 0 | 0.79705 |
| 385. | RGMB | NM_173670.2 | 1.744692748 | −0.08083442 | 3.88E−14 | 0.387433275 | 0 | 0.58277 |
| 386. | SNORD57 | NR_002738.1 | 1.743583957 | −0.13727561 | 3.02E−14 | 0.143871452 | 0 | 0.29888 |
| 387. | B3GNT2 | NM_006577.5 | 1.7399837 | 0.250805704 | 1.11E−14 | 0.008204737 | 0 | 0.03325 |
| 388. | RHPN2 | NM_033103.3 | 1.737884672 | 0.158675469 | 2.56E−15 | 0.058054713 | 0 | 0.15405 |
| 389. | YARS2 | NM_001040436.1 | 1.734675756 | 0.142331612 | 1.56E−14 | 0.116254974 | 0 | 0.25685 |
| 390. | SHISA2 | NM_001007538.1 | 1.730687009 | −0.29083242 | 2.65E−14 | 0.003885633 | 0 | 0.01799 |
| 391. | IRX5 | NM_005853.5 | 1.727349949 | 0.188078458 | 3.74E−14 | 0.050714653 | 0 | 0.13905 |
| 392. | ALG13 | NM_018466.3 | 1.725153512 | 0.27014979 | 1.71E−14 | 0.005461924 | 0 | 0.02376 |
| 393. | STAU1 | NM_017453.2 | 1.725139964 | 0.220644256 | 1.02E−14 | 0.016547772 | 0 | 0.05821 |
| 394. | EAF1 | NM_033083.6 | 1.723531505 | 0.009204673 | 1.84E−13 | 0.926295141 | 0 | 0.96428 |
| 395. | LOC440258 | NM_001013702.1 | 1.72092305 | 0.222949425 | 4.34E−14 | 0.023466916 | 0 | 0.07668 |
| 396. | HNRPA1P4 | XM_939887.2 | 1.720333337 | −1.13055551 | 7.21E−15 | 1.63E−11 | 0 | 0 |
| 397. | LOC730081 | XR_041261.1 | 1.720102785 | 0.285804464 | 5.43E−14 | 0.005563248 | 0 | 0.02411 |
| 398. | CDKN2AIP | NM_017632.2 | 1.718073072 | 0.331152557 | 5.13E−14 | 0.001738143 | 0 | 0.00916 |
| 399. | LOC440061 | XR_037839.1 | 1.712654514 | −0.25460059 | 3.16E−12 | 0.037315128 | 0 | 0.10986 |
| 400. | C16orf80 | NM_013242.2 | 1.710982277 | −0.18355681 | 2.36E−14 | 0.048676471 | 0 | 0.13456 |
| 401. | CTH | NM_153742.3 | 1.708483062 | 0.2684476 | 1.19E−14 | 0.004647673 | 0 | 0.02082 |
| 402. | DDX47 | NM_016355.3 | 1.708313699 | 0.248170569 | 1.77E−15 | 0.003940592 | 0 | 0.01819 |
| 403. | TFB2M | NM_022366.1 | 1.708281567 | −0.01288396 | 2.95E−14 | 0.88539985 | 0 | 0.94104 |
| 404. | C1orf52 | NM_198077.2 | 1.703202814 | −0.22109401 | 9.07E−16 | 0.006815725 | 0 | 0.02859 |
| 405. | C14orf138 | NM_001040662.1 | 1.702642937 | 0.300283903 | 1.34E−14 | 0.001999261 | 0 | 0.0103 |
| 406. | AURKAPS1 | NR_001587.1 | 1.70161228 | 0.068219051 | 2.34E−15 | 0.385267608 | 0 | 0.58075 |
| 407. | ARPP-21 | NM_001025068.1 | 1.69906582 | 0.013710129 | 3.79E−13 | 0.893086928 | 0 | 0.94527 |
| 408. | PRPF18 | NM_003675.3 | 1.698120259 | 0.343027256 | 2.69E−13 | 0.002458302 | 0 | 0.01222 |
| 409. | WDR43 | XM_944889.1 | 1.688207463 | 0.98248568 | 2.28E−14 | 4.03E−10 | 0 | 0 |
| 410. | SLC25A4 | NM_001151.2 | 1.68799987 | 0.228376088 | 2.32E−13 | 0.029717308 | 0 | 0.09193 |
| 411. | EIF4A3 | NM_014740.2 | 1.682073483 | −0.02293191 | 7.82E−15 | 0.779929294 | 0 | 0.88037 |
| 412. | SNORD68 | NR_002450.1 | 1.678911456 | 0.096404262 | 8.09E−15 | 0.247869843 | 0 | 0.43546 |
| 413. | LOC729423 | XM_001726952.1 | 1.67695359 | −0.81244327 | 4.52E−12 | 7.23E−07 | 0 | 1.00E−05 |
| 414. | MAP2 | NM_002374.3 | 1.676115893 | 1.955494491 | 3.97E−13 | 2.26E−14 | 0 | 0 |
| 415. | TUBB4Q | NM_020040.3 | 1.675304726 | −0.29288776 | 1.74E−13 | 0.006038937 | 0 | 0.02585 |
| 416. | MAD2L1BP | NM_014628.2 | 1.674485119 | 0.49354184 | 1.75E−14 | 1.05E−05 | 0 | 0.00013 |
| 417. | NUP98 | NM_016320.3 | 1.674194958 | −0.17098799 | 5.36E−15 | 0.042873219 | 0 | 0.12239 |
| 418. | TDG | NM_003211.3 | 1.674073464 | 0.174499498 | 1.04E−13 | 0.074107463 | 0 | 0.18496 |
| 419. | TCEB3 | NM_003198.1 | 1.673830159 | 0.528724598 | 4.16E−15 | 1.48E−06 | 0 | 2.00E−05 |
| 420. | PTP4A1 | NM_003463.3 | 1.672611158 | −0.12680128 | 2.38E−14 | 0.153505006 | 0 | 0.31292 |
| 421. | HSPA8 | NM_153201.1 | 1.666088959 | −0.34827432 | 1.87E−14 | 0.00051701 | 0 | 0.00332 |
| 422. | UBL5 | NM_024292.2 | 1.665307847 | −0.10865722 | 2.41E−13 | 0.272792946 | 0 | 0.4649 |
| 423. | C2orf56 | NM_001083946.1 | 1.661422458 | 0.281261857 | 2.36E−14 | 0.003507891 | 0 | 0.01649 |
| 424. | MAST4 | NM_198828.2 | 1.658879828 | 0.031824635 | 1.15E−14 | 0.700387125 | 0 | 0.82933 |
| 425. | BCL2L12 | NM_001040668.1 | 1.656287859 | −0.3354186 | 1.93E−14 | 0.000708935 | 0 | 0.00432 |
| 426. | INVS | NM_183245.1 | 1.652697508 | 0.431685941 | 1.42E−13 | 0.00016241 | 0 | 0.00124 |
| 427. | CDC42 | NM_001039802.1 | 1.650469411 | 0.562942841 | 4.05E−14 | 2.74E−06 | 0 | 4.00E−05 |
| 428. | ATF3 | NM_001040619.1 | 1.647975758 | 1.164189073 | 1.21E−13 | 6.55E−11 | 0 | 0 |
| 429. | HEY2 | NM_012259.1 | 1.643903866 | 0.097146095 | 7.56E−13 | 0.348571796 | 0 | 0.54507 |
| 430. | ZSWIM6 | XM_035299.8 | 1.642128642 | −0.4778082 | 1.46E−14 | 1.09E−05 | 0 | 0.00013 |
| 431. | C3orf58 | NM_173552.2 | 1.641296279 | 0.01647501 | 1.46E−12 | 0.87657807 | 0 | 0.93618 |
| 432. | LOC401097 | XM_941354.2 | 1.638403413 | −0.11820885 | 2.04E−13 | 0.222817497 | 0 | 0.40457 |
| 433. | EGLN1 | NM_022051.1 | 1.637555803 | 1.211267396 | 7.84E−14 | 1.92E−11 | 0 | 0 |
| 434. | CUGBP1 | NM_001025596.1 | 1.6340811 | 0.303511987 | 3.48E−14 | 0.001989256 | 0 | 0.01026 |
| 435. | LOC642678 | XM_926130.1 | 1.63220557 | 0.263521524 | 9.61E−15 | 0.003494982 | 0 | 0.01644 |
| 436. | C15orf60 | NM_001042367.1 | 1.630853946 | −0.02529098 | 4.33E−15 | 0.743037038 | 0 | 0.85707 |
| 437. | PHAX | NM_032177.2 | 1.630648972 | 0.499748672 | 1.47E−14 | 5.57E−06 | 0 | 7.00E−05 |
| 438. | MED6 | NM_005466.2 | 1.62829406 | 0.234613809 | 7.47E−15 | 0.007155742 | 0 | 0.02974 |
| 439. | CTNNAL1 | NM_003798.2 | 1.625720574 | −0.17578358 | 3.23E−14 | 0.050525454 | 0 | 0.13863 |
| 440. | PDK3 | NM_005391.2 | 1.625329362 | 0.772620864 | 4.98E−14 | 2.32E−08 | 0 | 0 |
| 441. | PLEKHB2 | NM_001031706.1 | 1.622273401 | 0.054655486 | 3.21E−14 | 0.523709551 | 0 | 0.70026 |
| 442. | NUDT11 | NM_018159.3 | 1.621197373 | −0.57819231 | 2.56E−15 | 1.73E−07 | 0 | 0 |
| 443. | BDNF | NM_170732.3 | 1.620223761 | 0.035311781 | 1.84E−11 | 0.769673882 | 0 | 0.8738 |
| 444. | NGLY1 | NM_018297.2 | 1.61360189 | 0.298383033 | 2.22E−15 | 0.000564863 | 0 | 0.00357 |
| 445. | ZNF705D | NM_001039615.3 | 1.610202541 | −0.02597342 | 5.30E−13 | 0.792068462 | 0 | 0.88765 |
| 446. | MED15 | NM_001003891.1 | 1.609997025 | −0.06790583 | 9.68E−14 | 0.452731449 | 0 | 0.64195 |
| 447. | ZIK1 | NM_001010879.2 | 1.609559281 | 0.460511424 | 6.70E−14 | 3.67E−05 | 0 | 0.00036 |
| 448. | PRPF40A | NM_017892.3 | 1.60701676 | 0.43524072 | 1.48E−14 | 2.68E−05 | 0 | 0.00027 |
| 449. | PMAIP1 | NM_021127.1 | 1.605658964 | 0.635854476 | 1.01E−10 | 9.09E−05 | 0 | 0.00076 |
| 450. | KRTAP2-1 | XM_926554.2 | 1.605615798 | −0.00836408 | 2.52E−12 | 0.937638775 | 0 | 0.96996 |
| 451. | SNORA67 | NR_002912.1 | 1.603876448 | −0.01990015 | 4.24E−13 | 0.837308572 | 0 | 0.9142 |
| 452. | NKIRAS1 | NM_020345.3 | 1.602380221 | −0.21505265 | 7.40E−15 | 0.011263604 | 0 | 0.04272 |
| 453. | SAMD8 | NM_144660.1 | 1.599343462 | 0.673947538 | 1.53E−12 | 2.24E−06 | 0 | 3.00E−05 |
| 454. | LOC728408 | XR_039142.1 | 1.597556992 | 0.195973187 | 1.22E−13 | 0.039933909 | 0 | 0.11598 |
| 455. | MAPKAP1 | NM_001006618.1 | 1.596744892 | 0.290086205 | 3.92E−15 | 0.000881923 | 0 | 0.0052 |
| 456. | ECD | NM_007265.1 | 1.596500869 | 0.440442679 | 6.46E−13 | 0.000216775 | 0 | 0.00159 |
| 457. | TOPORS | NM_005802.2 | 1.595626451 | 0.418698719 | 3.87E−15 | 1.65E−05 | 0 | 0.00018 |
| 458. | RTN4 | NM_007008.2 | 1.593944683 | 0.264653804 | 4.43E−13 | 0.011750635 | 0 | 0.04419 |
| 459. | ARHGAP19 | NM_032900.4 | 1.593323373 | −0.26239597 | 8.31E−14 | 0.006924484 | 0 | 0.02897 |
| 460. | SH3GL2 | NM_003026.1 | 1.59238137 | 0.073484379 | 2.15E−13 | 0.431704365 | 0 | 0.62263 |
| 461. | MYBPH | NM_004997.2 | 1.591566604 | −0.44577378 | 2.98E−11 | 0.001408545 | 0 | 0.00767 |
| 462. | NT5DC3 | NM_016575.1 | 1.590953067 | −0.47059679 | 2.60E−15 | 2.60E−06 | 0 | 4.00E−05 |
| 463. | SNRPN | NM_022807.2 | 1.589618988 | −0.17582455 | 1.07E−13 | 0.059789988 | 0 | 0.15751 |
| 464. | GJA1 | NM_000165.3 | 1.58780493 | −0.178685 | 1.61E−12 | 0.094898465 | 0 | 0.22167 |
| 465. | LOC400013 | XR_039228.1 | 1.587250344 | 0.031497834 | 6.92E−15 | 0.682627414 | 0 | 0.81743 |
| 466. | HNRNPM | NM_031203.2 | 1.586899323 | −0.14982292 | 1.50E−14 | 0.072940459 | 0 | 0.1828 |
| 467. | STX3 | NM_004177.3 | 1.581157146 | 0.356650082 | 3.87E−13 | 0.00114871 | 0 | 0.00645 |
| 468. | LOC644914 | XM_930111.2 | 1.575423572 | −0.06923633 | 6.93E−09 | 0.676335846 | 0 | 0.81295 |
| 469. | LOC100133836 | XM_001713608.1 | 1.575168029 | 0.10564662 | 8.84E−13 | 0.292962838 | 0 | 0.48767 |
| 470. | GTF2B | NM_001514.3 | 1.574967456 | 0.347163622 | 1.99E−15 | 8.61E−05 | 0 | 0.00073 |
| 471. | SCML1 | NM_001037540.1 | 1.574201734 | 0.188494795 | 7.40E−13 | 0.065842365 | 0 | 0.16936 |
| 472. | TBPL1 | NM_004865.2 | 1.573806979 | 0.205833238 | 8.90E−14 | 0.02721547 | 0 | 0.08586 |
| 473. | ZNF551 | NM_138347.2 | 1.568648262 | −0.00011162 | 7.49E−13 | 0.999088618 | 0 | 0.99958 |
| 474. | SFRS10 | NM_004593.1 | 1.5681537 | −0.75201357 | 4.24E−14 | 1.76E−08 | 0 | 0 |
| 475. | OXR1 | NM_181354.3 | 1.562540281 | −0.71933778 | 1.96E−14 | 1.74E−08 | 0 | 0 |
| 476. | BHLHE22 | NM_152414.3 | 1.560252215 | 0.101626343 | 2.45E−13 | 0.274055287 | 0 | 0.46656 |
| 477. | OR6X1 | NM_001005188.1 | 1.558631453 | −0.12817436 | 3.38E−13 | 0.178290073 | 0 | 0.34676 |
| 478. | LMO4 | NM_006769.2 | 1.557490718 | 0.353184397 | 4.70E−14 | 0.000377316 | 0 | 0.00254 |
| 479. | LOC645166 | XM_001129441.2 | 1.554857161 | 0.192882939 | 2.99E−14 | 0.026452044 | 0 | 0.08411 |
| 480. | STIL | NM_003035.2 | 1.55295308 | −0.4828307 | 5.72E−14 | 1.19E−05 | 0 | 0.00014 |
| 481. | LOC100133760 | XM_001719676.1 | 1.552936795 | 0.16059717 | 1.79E−13 | 0.084458299 | 0 | 0.20408 |
| 482. | FZD7 | NM_003507.1 | 1.550854894 | −0.13090274 | 2.74E−13 | 0.16304396 | 0 | 0.32612 |
| 483. | BTAF1 | NM_003972.2 | 1.549813547 | 0.070013752 | 6.30E−13 | 0.467209265 | 0 | 0.65411 |
| 484. | CCNC | NM_005190.3 | 1.54954803 | 0.095974423 | 1.98E−14 | 0.235071298 | 0 | 0.41982 |
| 485. | DNAJB1 | NM_006145.1 | 1.54841722 | −0.33172027 | 3.62E−14 | 0.000572756 | 0 | 0.00361 |
| 486. | ASB7 | NM_024708.2 | 1.548080129 | 0.274794966 | 3.54E−14 | 0.002879842 | 0 | 0.01399 |
| 487. | DDX39 | NM_005804.2 | 1.547089571 | 0.1754289 | 2.56E−13 | 0.065048264 | 0 | 0.16785 |
| 488. | LOC85389 | NR_001453.1 | 1.546684873 | 0.007015657 | 1.79E−12 | 0.944650057 | 0 | 0.97324 |
| 489. | APIP | NM_015957.1 | 1.542886454 | −0.00581131 | 4.45E−15 | 0.936540677 | 0 | 0.96961 |
| 490. | ZNF330 | NM_014487.3 | 1.540957622 | −0.07386804 | 2.12E−13 | 0.413978578 | 0 | 0.60714 |
| 491. | ABL1 | NM_007313.2 | 1.539307052 | 0.145489802 | 4.48E−13 | 0.130010749 | 0 | 0.27803 |
| 492. | WDR45L | NM_019613.2 | 1.538237535 | 0.360400213 | 7.47E−14 | 0.000349259 | 0 | 0.00237 |
| 493. | CDR2 | NM_001802.1 | 1.53742338 | −0.30870921 | 9.68E−14 | 0.001642031 | 0 | 0.00874 |
| 494. | LOC648040 | XM_937090.1 | 1.53646268 | 0.053464634 | 5.12E−13 | 0.570118838 | 0 | 0.73692 |
| 495. | TMSB15A | NM_021992.2 | 1.535257062 | −1.12870839 | 6.88E−14 | 1.89E−11 | 0 | 0 |
| 496. | DDX3X | NM_001356.3 | 1.533546299 | −0.1679824 | 1.05E−12 | 0.096033914 | 0 | 0.2235 |
| 497. | TAF5 | NM_006951.3 | 1.533278341 | −0.20722919 | 1.52E−13 | 0.026663375 | 0 | 0.0846 |
| 498. | SNORA80 | NR_002996.2 | 1.531309838 | 0.08504288 | 4.65E−14 | 0.306803596 | 0 | 0.5021 |
| 499. | LOC731049 | XM_001129232.1 | 1.530043889 | −0.70866978 | 7.11E−12 | 1.95E−06 | 0 | 3.00E−05 |
| 500. | RBM14 | NM_006328.2 | 1.527566336 | −0.21778628 | 2.56E−14 | 0.011666847 | 0 | 0.04393 |
| 501. | CNNM4 | NM_020184.3 | 1.523298011 | 0.02269985 | 1.51E−12 | 0.818049687 | 0 | 0.90311 |
| 502. | GLS | NM_014905.2 | 1.52096024 | −0.80631784 | 2.48E−14 | 2.12E−09 | 0 | 0 |
| 503. | METTL7B | NM_152637.1 | 1.518189623 | −0.55481484 | 4.95E−13 | 6.63E−06 | 0 | 9.00E−05 |
| 504. | RSRC2 | NM_023012.4 | 1.514721977 | −0.1150749 | 1.90E−14 | 0.148027453 | 0 | 0.30524 |
| 505. | FAM90A17 | XM_001129363.2 | 1.513614783 | −0.09546518 | 1.31E−13 | 0.273354754 | 0 | 0.46568 |
| 506. | SLU7 | NM_006425.4 | 1.512152853 | 0.717990416 | 3.02E−14 | 1.54E−08 | 0 | 0 |
| 507. | LOC654256 | XM_942353.1 | 1.509691066 | 0.003681464 | 2.30E−12 | 0.970637014 | 0 | 0.98654 |
| 508. | LOC730820 | XM_001127763.1 | 1.506653829 | 0.257075032 | 7.79E−14 | 0.005290665 | 0 | 0.02314 |
| 509. | GLMN | NM_053274.2 | 1.506236955 | −0.16030238 | 1.21E−12 | 0.107692491 | 0 | 0.24313 |
| 510. | LOC728640 | XR_015400.1 | 1.506174187 | −0.16289752 | 1.38E−13 | 0.068584855 | 0 | 0.17462 |
| 511. | NUP54 | NM_017426.2 | 1.503392773 | 0.027683096 | 2.48E−13 | 0.753930426 | 0 | 0.8649 |
| 512. | AHR | NM_001621.3 | 1.500923505 | 1.64275074 | 4.05E−13 | 7.61E−14 | 0 | 0 |
| 513. | H2AFZ | NM_002106.3 | 1.499572269 | −0.3879234 | 9.97E−13 | 0.000523412 | 0 | 0.00335 |
| 514. | C1orf128 | NM_020362.3 | 1.497934427 | −0.06705701 | 1.71E−14 | 0.383082842 | 0 | 0.57869 |
| 515. | RNF4 | NM_002938.2 | 1.497336031 | −0.25222173 | 1.90E−13 | 0.008011302 | 0 | 0.03261 |
| 516. | TFAP2C | NM_003222.3 | 1.496945403 | −0.18736367 | 5.78E−13 | 0.052500579 | 0 | 0.14285 |
| 517. | C1orf185 | XM_209252.6 | 1.49683368 | 0.058034061 | 3.70E−11 | 0.617231268 | 0 | 0.77232 |
| 518. | BRD2 | NM_005104.2 | 1.495026143 | 0.083794647 | 2.68E−14 | 0.288647235 | 0 | 0.48332 |
| 519. | RAB3IP | NM_175624.2 | 1.494489505 | 0.11802541 | 3.56E−13 | 0.196543599 | 0 | 0.37153 |
| 520. | PITX1 | NM_002653.3 | 1.494464327 | 0.245507893 | 7.06E−13 | 0.014555822 | 0 | 0.05244 |
| 521. | ALG11 | NM_001004127.1 | 1.493839221 | 0.493936201 | 1.03E−12 | 3.81E−05 | 0 | 0.00037 |
| 522. | TCP1 | NM_030752.2 | 1.493545632 | −0.05603384 | 4.05E−12 | 0.584510317 | 0 | 0.74675 |
| 523. | DHX9 | NM_001357.3 | 1.493186665 | −0.0171031 | 2.72E−13 | 0.846033265 | 0 | 0.9184 |
| 524. | NOL11 | NM_015462.3 | 1.489057392 | −0.04128462 | 8.08E−14 | 0.616750218 | 0 | 0.77192 |
| 525. | TPM3 | NM_152263.2 | 1.48876168 | 0.579968022 | 3.03E−13 | 1.95E−06 | 0 | 3.00E−05 |
| 526. | LOC283116 | XM_208043.4 | 1.487827846 | 0.118511077 | 1.06E−12 | 0.218730077 | 0 | 0.39975 |
| 527. | RYBP | NM_012234.4 | 1.486399459 | −0.35369721 | 2.11E−14 | 0.000147008 | 0 | 0.00114 |
| 528. | SNORD43 | NR_002439.1 | 1.48537347 | 0.039488281 | 2.52E−11 | 0.725753871 | 0 | 0.84571 |
| 529. | RHOBTB1 | NM_198225.1 | 1.485256007 | −0.45165823 | 2.03E−13 | 3.61E−05 | 0 | 0.00035 |
| 530. | LOC641802 | XM_935872.1 | 1.485216463 | 0.376747849 | 8.91E−14 | 0.000169723 | 0 | 0.00128 |
| 531. | HTRA4 | NM_153692.2 | 1.484230449 | 0.215030394 | 1.08E−13 | 0.016696826 | 0 | 0.05858 |
| 532. | ZNF263 | NM_005741.3 | 1.481056926 | −0.13718839 | 3.70E−14 | 0.09197961 | 0 | 0.21695 |
| 533. | LOC645232 | XM_928271.1 | 1.479784917 | −0.00044634 | 2.41E−12 | 0.996376666 | 0 | 0.99841 |
| 534. | DI03 | NM_001362.2 | 1.477091363 | −0.07106955 | 1.13E−11 | 0.508479304 | 0 | 0.68767 |
| 535. | SRFBP1 | NM_152546.1 | 1.475739204 | 0.45287234 | 7.60E−14 | 1.70E−05 | 0 | 0.00019 |
| 536. | DNAJA1 | NM_001539.2 | 1.471203017 | 0.643687358 | 8.61E−14 | 1.34E−07 | 0 | 0 |
| 537. | PPFIBP2 | NM_003621.1 | 1.467327061 | −0.00235925 | 8.11E−12 | 0.981938862 | 0 | 0.99193 |
| 538. | NDEL1 | NM_030808.3 | 1.467127006 | −0.02163182 | 2.46E−14 | 0.776179739 | 0 | 0.87787 |
| 539. | RRP15 | NM_016052.3 | 1.465748171 | 0.165874388 | 2.22E−14 | 0.038048251 | 0 | 0.11152 |
| 540. | SUPT6H | NM_003170.3 | 1.464959001 | 0.257955364 | 1.11E−11 | 0.022964207 | 0 | 0.07531 |
| 541. | EIF1 | NM_005801.3 | 1.46472025 | −0.21791089 | 7.47E−13 | 0.025400915 | 0 | 0.08167 |
| 542. | C1orf187 | NM_198545.2 | 1.46296546 | −0.02269906 | 3.13E−13 | 0.794138153 | 0 | 0.88916 |
| 543. | SLC35F3 | NM_173508.2 | 1.462545631 | −0.09338754 | 3.92E−11 | 0.414528519 | 0 | 0.60764 |
| 544. | LOC732387 | XR_015868.1 | 1.45974406 | −0.16936331 | 4.12E−12 | 0.101560033 | 0 | 0.23264 |
| 545. | DPPA3 | NM_199286.2 | 1.457269792 | −0.00424754 | 3.09E−11 | 0.969625546 | 0 | 0.98648 |
| 546. | BCCIP | NM_078468.1 | 1.454741172 | 0.108769507 | 1.55E−13 | 0.2007215 | 0 | 0.3768 |
| 547. | FBXW7 | NM_033632.2 | 1.454071129 | 0.143817093 | 1.36E−12 | 0.135509671 | 0 | 0.2859 |
| 548. | LOC732360 | XR_038607.1 | 1.450256538 | 0.156874564 | 3.06E−13 | 0.079962819 | 0 | 0.19619 |
| 549. | ILF2 | NM_004515.2 | 1.450144347 | −0.2235945 | 6.27E−14 | 0.009657131 | 0 | 0.03776 |
| 550. | TAF7 | NM_005642.2 | 1.44931134 | 0.260318224 | 2.95E−12 | 0.013665045 | 0 | 0.05007 |
| 551. | FBXO28 | NM_015176.1 | 1.449224666 | 0.332557144 | 8.84E−14 | 0.000468708 | 0 | 0.00306 |
| 552. | LOC648390 | XR_037845.1 | 1.446477421 | −0.16743094 | 9.36E−14 | 0.048260154 | 0 | 0.13375 |
| 553. | LOC100134083 | XM_001714551.1 | 1.444757278 | 0.065916236 | 7.44E−11 | 0.572248293 | 0 | 0.73805 |
| 554. | CDS1 | NM_001263.2 | 1.443156315 | −0.03729718 | 1.11E−10 | 0.753883222 | 0 | 0.8649 |
| 555. | TMPO | NM_003276.1 | 1.440849718 | −0.16059408 | 6.25E−12 | 0.122797864 | 0 | 0.26716 |
| 556. | SLC10A4 | NM_152679.2 | 1.438316891 | 0.131702245 | 1.31E−12 | 0.164547852 | 0 | 0.32843 |
| 557. | SFRS2 | NM_003016.3 | 1.43818754 | −0.55216219 | 5.75E−14 | 6.74E−07 | 0 | 1.00E−05 |
| 558. | LOC100129267 | XR_037397.1 | 1.436698179 | −0.10192887 | 9.54E−13 | 0.268686055 | 0 | 0.46016 |
| 559. | CSRP2 | NM_001321.1 | 1.436294732 | −0.82970582 | 1.34E−12 | 1.78E−08 | 0 | 0 |
| 560. | HNRNPA2B1 | NM_031243.2 | 1.436197608 | 0.130215149 | 4.15E−13 | 0.143912138 | 0 | 0.29891 |
| 561. | CLP1 | NM_006831.1 | 1.433554015 | 0.348572252 | 2.57E−13 | 0.000467471 | 0 | 0.00305 |
| 562. | MTF2 | NM_007358.2 | 1.430806129 | 0.244039964 | 5.37E−13 | 0.010617995 | 0 | 0.04071 |
| 563. | C13orf27 | NM_138779.2 | 1.428664756 | 0.047576445 | 1.17E−11 | 0.646885227 | 0 | 0.79308 |
| 564. | MEX3C | NM_016626.3 | 1.427435993 | 0.093724078 | 4.05E−13 | 0.283008406 | 0 | 0.47662 |
| 565. | CBX4 | NM_003655.2 | 1.426881488 | 0.311472696 | 2.28E−13 | 0.001191417 | 0 | 0.00666 |
| 566. | DDX21 | NM_004728.2 | 1.426501707 | −0.17094795 | 1.18E−13 | 0.043877499 | 0 | 0.12445 |
| 567. | SFRS15 | NM_020706.1 | 1.424894099 | 0.168074451 | 2.26E−13 | 0.054322289 | 0 | 0.14667 |
| 568. | AHCTF1 | NM_015446.3 | 1.422776124 | 0.285495925 | 5.55E−14 | 0.001279213 | 0 | 0.00706 |
| 569. | STARD7 | NM_020151.3 | 1.422197808 | 0.009352233 | 7.93E−12 | 0.926146793 | 0 | 0.96426 |
| 570. | LOC347376 | XM_937928.1 | 1.421140581 | −0.31817931 | 2.93E−09 | 0.034321047 | 0 | 0.10284 |
| 571. | C6orf66 | NM_014165.1 | 1.417175337 | 0.119941892 | 4.37E−13 | 0.172177225 | 0 | 0.33865 |
| 572. | LOC644330 | XR_017492.1 | 1.414996029 | 0.663508029 | 1.07E−10 | 1.26E−05 | 0 | 0.00015 |
| 573. | ABCG1 | NM_207629.1 | 1.411683724 | −0.03110394 | 1.60E−11 | 0.765467774 | 0 | 0.87176 |
| 574. | NOV | NM_002514.2 | 1.410494135 | −0.04872585 | 1.01E−11 | 0.631961219 | 0 | 0.7829 |
| 575. | GFM1 | NM_024996.5 | 1.407835107 | −0.79555703 | 2.13E−13 | 4.99E−09 | 0 | 0 |
| 576. | LOC652595 | XM_942117.1 | 1.405139131 | −0.09810807 | 1.65E−12 | 0.290150795 | 0 | 0.48482 |
| 577. | ZNF281 | NM_012482.3 | 1.404648603 | −0.00361308 | 7.00E−13 | 0.966941631 | 0 | 0.98526 |
| 578. | ARID4B | NM_016374.5 | 1.404306999 | 0.569331433 | 8.04E−12 | 1.25E−05 | 0 | 0.00015 |
| 579. | LOC645233 | NR_024382.1 | 1.403953598 | 0.103826446 | 1.83E−12 | 0.266012715 | 0 | 0.45717 |
| 580. | MYOG | NM_002479.4 | 1.400086822 | −0.85712385 | 5.19E−10 | 1.19E−06 | 0 | 2.00E−05 |
| 581. | OSR2 | XM_001126824.1 | 1.399885028 | 0.140060681 | 4.11E−13 | 0.108689759 | 0 | 0.24479 |
| 582. | LOC727758 | XM_001125808.2 | 1.396320204 | 0.343226006 | 2.86E−12 | 0.001407708 | 0 | 0.00766 |
| 583. | HIC2 | NM_015094.2 | 1.395105507 | −0.07986711 | 7.32E−14 | 0.303989988 | 0 | 0.49905 |
| 584. | PHLPP2 | NM_015020.2 | 1.393342712 | 0.011230089 | 1.82E−13 | 0.888929658 | 0 | 0.94301 |
| 585. | LOC728153 | XM_001128002.1 | 1.392470561 | −0.1239819 | 2.15E−12 | 0.187228802 | 0 | 0.35968 |
| 586. | PHLPP1 | NM_194449.1 | 1.391942682 | −0.11204861 | 1.71E−13 | 0.171819649 | 0 | 0.33813 |
| 587. | SBNO1 | NM_018183.2 | 1.391640047 | 0.912611144 | 3.73E−11 | 4.46E−08 | 0 | 0 |
| 588. | ZNF574 | NM_022752.5 | 1.391080303 | −0.02575443 | 9.43E−13 | 0.769404142 | 0 | 0.87366 |
| 589. | BAGE5 | NM_182484.1 | 1.390112085 | 0.312869895 | 1.32E−12 | 0.002062769 | 0 | 0.01058 |
| 590. | EML4 | NM_019063.2 | 1.388253958 | −0.89138642 | 1.80E−12 | 4.14E−09 | 0 | 0 |
| 591. | SHFM1 | NM_006304.1 | 1.387446578 | −0.02974983 | 3.90E−12 | 0.753525894 | 0 | 0.86487 |
| 592. | SLC12A2 | NM_001046.2 | 1.386367245 | −0.75596 | 1.01E−11 | 2.46E−07 | 0 | 1.00E−05 |
| 593. | RND3 | NM_005168.3 | 1.386186726 | 0.925453193 | 4.07E−12 | 4.56E−09 | 0 | 0 |
| 594. | MGAT4C | NM_013244.2 | 1.385868579 | −0.0799553 | 2.07E−12 | 0.385732105 | 0 | 0.58121 |
| 595. | ERN1 | NM_152461.2 | 1.385516021 | −0.07639676 | 1.49E−12 | 0.398333442 | 0 | 0.59217 |
| 596. | C16orf87 | NM_001001436.2 | 1.384283092 | −0.05230872 | 1.30E−12 | 0.557876212 | 0 | 0.72752 |
| 597. | MBIP | NM_016586.1 | 1.383473795 | 0.279754309 | 3.55E−13 | 0.002746197 | 0 | 0.0134 |
| 598. | SUV420H1 | NM_016028.4 | 1.383227676 | 0.315044495 | 2.43E−12 | 0.00245823 | 0 | 0.01222 |
| 599. | MFSD4 | NM_181644.2 | 1.383119443 | −0.12045834 | 4.50E−12 | 0.214393959 | 0 | 0.39439 |
| 600. | TSC22D2 | NM_014779.2 | 1.382388949 | −0.27410124 | 5.22E−11 | 0.019370365 | 0 | 0.06592 |
| 601. | FNBP1L | NM_001024948.1 | 1.382235187 | −0.88872668 | 1.36E−12 | 3.13E−09 | 0 | 0 |
| 602. | SLC25A13 | NM_014251.1 | 1.382003256 | −0.09193427 | 1.14E−12 | 0.30332903 | 0 | 0.49844 |
| 603. | GAB2 | NM_080491.1 | 1.381903767 | −0.22970672 | 5.75E−13 | 0.012715227 | 0 | 0.04719 |
| 604. | BMP2K | NM_017593.3 | 1.380005052 | 0.155131234 | 3.90E−12 | 0.110918545 | 0 | 0.24845 |
| 605. | CCK | NM_000729.3 | 1.377654752 | −0.04697335 | 9.98E−14 | 0.543484917 | 0 | 0.71617 |
| 606. | GCC1 | NM_024523.5 | 1.377403631 | 0.345129567 | 3.16E−13 | 0.000388553 | 0 | 0.00261 |
| 607. | DOHH | NM_031304.3 | 1.377136426 | 0.060583272 | 3.42E−13 | 0.464259653 | 0 | 0.65186 |
| 608. | ZNF721 | NM_133474.2 | 1.374804369 | −0.08379608 | 2.03E−12 | 0.359423741 | 0 | 0.55546 |
| 609. | MGC39900 | NM_194324.1 | 1.373970776 | −0.60573503 | 7.17E−13 | 6.56E−07 | 0 | 1.00E−05 |
| 610. | KCTD5 | NM_018992.2 | 1.373520781 | −0.24714681 | 2.17E−14 | 0.002096705 | 0 | 0.01073 |
| 611. | CDO1 | NM_001801.2 | 1.372706326 | 0.115369911 | 3.98E−13 | 0.172921753 | 0 | 0.33967 |
| 612. | SNHG1 | NR_003098.1 | 1.372510324 | −0.24948274 | 8.72E−14 | 0.003479011 | 0 | 0.01638 |
| 613. | RPF1 | NM_025065.6 | 1.372051665 | 0.218421831 | 8.46E−13 | 0.018398193 | 0 | 0.06336 |
| 614. | ZNF408 | NM_024741.1 | 1.371029586 | 0.061624875 | 2.76E−13 | 0.449623929 | 0 | 0.63904 |
| 615. | PFKFB3 | NM_004566.2 | 1.369564991 | 0.250778898 | 8.85E−13 | 0.00802405 | 0 | 0.03265 |
| 616. | C8orf79 | NM_020844.2 | 1.369321582 | 0.053699642 | 1.69E−11 | 0.597484886 | 0 | 0.75758 |
| 617. | ZNF256 | NM_005773.2 | 1.367327098 | 0.093687711 | 2.44E−12 | 0.309062419 | 0 | 0.50444 |
| 618. | VGLL2 | NM_153453.1 | 1.367173619 | 0.126611023 | 1.56E−12 | 0.163851344 | 0 | 0.32735 |
| 619. | CCDC49 | NM_017748.3 | 1.364719391 | 0.136302741 | 5.90E−13 | 0.11587493 | 0 | 0.25639 |
| 620. | AMD1 | NM_001033059.1 | 1.362028458 | −0.75667482 | 5.49E−12 | 1.11E−07 | 0 | 0 |
| 621. | RAPGEF2 | NM_014247.2 | 1.361231387 | 0.055181721 | 2.93E−10 | 0.642357037 | 0 | 0.79015 |
| 622. | SNORD36A | NR_002448.1 | 1.35930066 | 0.194368066 | 2.93E−09 | 0.162157825 | 0 | 0.32489 |
| 623. | BUD31 | NM_003910.2 | 1.357782326 | 0.193119675 | 5.33E−13 | 0.029005221 | 0 | 0.09028 |
| 624. | FBXL12 | NM_017703.1 | 1.356779609 | 0.167789916 | 1.68E−11 | 0.106231056 | 0 | 0.24053 |
| 625. | SNORD55 | NR_000015.2 | 1.356349401 | 0.150448175 | 5.67E−13 | 0.082334618 | 0 | 0.20033 |
| 626. | KIAA1429 | NM_183009.1 | 1.355815619 | 0.116182435 | 2.49E−13 | 0.154973264 | 0 | 0.31506 |
| 627. | LOC729200 | XR_015946.2 | 1.353808445 | 0.172309846 | 1.44E−12 | 0.059946156 | 0 | 0.15783 |
| 628. | DLEU1 | NR_002605.1 | 1.352475748 | −0.34822451 | 5.08E−13 | 0.000383086 | 0 | 0.00258 |
| 629. | BAZ1A | NM_013448.2 | 1.352043552 | 0.403141817 | 2.65E−13 | 5.40E−05 | 0 | 0.00049 |
| 630. | TXNDC12 | NM_015913.2 | 1.351366565 | −0.2192402 | 1.23E−12 | 0.01857404 | 0 | 0.06389 |
| 631. | SDC2 | NM_002998.3 | 1.350110395 | −0.08724225 | 1.56E−12 | 0.325167328 | 0 | 0.52177 |
| 632. | ROCK1 | NM_005406.2 | 1.34927666 | 0.148525788 | 3.50E−12 | 0.116008557 | 0 | 0.25657 |
| 633. | IER2 | NM_004907.2 | 1.349204584 | 0.064050022 | 2.24E−10 | 0.581064955 | 0 | 0.74435 |
| 634. | MRPL44 | NM_022915.2 | 1.348983987 | 0.617239832 | 4.79E−13 | 2.75E−07 | 0 | 1.00E−05 |
| 635. | HIST2H2BE | NM_003528.2 | 1.347350076 | 0.271284389 | 1.02E−13 | 0.001642357 | 0 | 0.00874 |
| 636. | SRP14P1 | NR_003273.1 | 1.345710532 | 0.171734899 | 2.29E−12 | 0.065389222 | 0 | 0.16848 |
| 637. | C13orf15 | NM_014059.2 | 1.345166905 | −0.26286642 | 1.58E−12 | 0.006411715 | 0 | 0.02713 |
| 638. | CRHBP | NM_001882.3 | 1.344139949 | −0.12281419 | 8.79E−12 | 0.209617367 | 0 | 0.38827 |
| 639. | LOC642333 | XR_019071.1 | 1.343692626 | 0.025090359 | 1.16E−11 | 0.796766364 | 0 | 0.8907 |
| 640. | KIAA0922 | NM_015196.2 | 1.342982901 | 0.04361988 | 3.17E−12 | 0.631042252 | 0 | 0.78227 |
| 641. | CAMK2G | NM_001222.2 | 1.341779485 | −0.17192793 | 4.52E−12 | 0.073983174 | 0 | 0.18471 |
| 642. | TNFRSF10D | NM_003840.3 | 1.340389565 | 0.140679104 | 1.35E−11 | 0.161604163 | 0 | 0.32404 |
| 643. | TC2N | NM_152332.3 | 1.338002738 | −0.12357892 | 2.28E−11 | 0.228501198 | 0 | 0.41174 |
| 644. | FBX044 | NM_001014765.1 | 1.334510208 | −0.03205932 | 9.53E−11 | 0.768688418 | 0 | 0.87335 |
| 645. | C1orf182 | NM_144627.2 | 1.333386659 | 0.214999181 | 7.85E−12 | 0.032329912 | 0 | 0.09824 |
| 646. | NRBF2 | NM_030759.3 | 1.330837635 | 0.047479638 | 2.06E−12 | 0.589362968 | 0 | 0.75086 |
| 647. | TMEM119 | NM_181724.1 | 1.330166389 | −0.5390834 | 9.96E−11 | 6.95E−05 | 0 | 0.00061 |
| 648. | TFAM | NM_003201.1 | 1.32791716 | −0.2004992 | 1.61E−12 | 0.029119131 | 0 | 0.09055 |
| 649. | ADNP2 | NM_014913.2 | 1.326077627 | −0.11379936 | 3.31E−13 | 0.160510429 | 0 | 0.32288 |
| 650. | DUX4 | NM_033178.1 | 1.325834384 | −0.12775373 | 6.03E−12 | 0.178056067 | 0 | 0.34639 |
| 651. | LOC100132418 | XM_001719607.1 | 1.325451904 | 0.072319727 | 2.91E−13 | 0.361714474 | 0 | 0.55754 |
| 652. | FAM89A | XM_939093.1 | 1.324823585 | 0.485070974 | 2.01E−11 | 7.88E−05 | 0 | 0.00068 |
| 653. | DOPEY1 | NM_015018.2 | 1.324238908 | −0.00463821 | 9.57E−13 | 0.955758726 | 0 | 0.97971 |
| 654. | RPS7 | NM_001011.3 | 1.323525637 | 0.022583587 | 1.80E−12 | 0.794199811 | 0 | 0.88916 |
| 655. | LOC285407 | XM_209597.8 | 1.321959178 | −0.17369245 | 3.54E−10 | 0.146378753 | 0 | 0.30272 |
| 656. | TRIM36 | NM_018700.3 | 1.321275285 | 0.009470146 | 8.39E−11 | 0.929501366 | 0 | 0.96595 |
| 657. | C5orf27 | XR_040299.1 | 1.320540369 | 0.154366501 | 1.77E−09 | 0.23549402 | 0 | 0.42037 |
| 658. | FAM53C | NM_016605.1 | 1.319296494 | −0.17615807 | 5.63E−12 | 0.066492472 | 0 | 0.17066 |
| 659. | ACAP2 | NM_012287.4 | 1.317695146 | −0.40479958 | 4.99E−12 | 0.000227632 | 0 | 0.00166 |
| 660. | LOC653080 | XM_925939.1 | 1.316954224 | −0.01245615 | 6.77E−12 | 0.893022129 | 0 | 0.94527 |
| 661. | NEDD4 | NM_006154.2 | 1.316933474 | −0.03632561 | 6.83E−12 | 0.695551993 | 0 | 0.82619 |
| 662. | RBM7 | NM_016090.2 | 1.315671181 | 0.056243381 | 1.80E−11 | 0.566438271 | 0 | 0.7341 |
| 663. | HIST1H2BK | NM_080593.1 | 1.315332973 | 1.001473221 | 4.10E−13 | 5.61E−11 | 0 | 0 |
| 664. | LOC728779 | XM_001128458.2 | 1.313895474 | −0.22508026 | 1.45E−10 | 0.051226883 | 0 | 0.14009 |
| 665. | SF3B4 | NM_005850.3 | 1.313732124 | 0.02305885 | 2.28E−12 | 0.791135393 | 0 | 0.887 |
| 666. | SNORD95 | NR_002591.1 | 1.310849643 | 0.196557485 | 5.32E−13 | 0.022164576 | 0 | 0.07311 |
| 667. | HIST2H2AC | NM_003517.2 | 1.310148122 | 0.96312555 | 1.46E−12 | 3.47E−10 | 0 | 0 |
| 668. | NCOA7 | NM_181782.2 | 1.308543286 | −0.05714471 | 1.26E−12 | 0.498253253 | 0 | 0.67988 |
| 669. | SNORD35A | NR_000018.1 | 1.308528091 | −0.11569171 | 4.01E−11 | 0.262839706 | 0 | 0.45307 |
| 670. | HIST2H2AA4 | NM_001040874.1 | 1.308519368 | 1.065247493 | 1.96E−13 | 8.39E−12 | 0 | 0 |
| 671. | POLB | NM_002690.1 | 1.307319155 | −0.10916113 | 1.26E−13 | 0.150760489 | 0 | 0.30898 |
| 672. | USPL1 | NM_005800.3 | 1.306587916 | 0.105879184 | 6.19E−12 | 0.254825898 | 0 | 0.4433 |
| 673. | PPP2R5E | NM_006246.2 | 1.306086979 | −0.22178951 | 5.16E−13 | 0.010766342 | 0 | 0.04116 |
| 674. | APOE | NM_000041.2 | 1.303237847 | −0.2177197 | 5.23E−08 | 0.171345308 | 0 | 0.3376 |
| 675. | YY1 | NM_003403.3 | 1.303013147 | 0.502718394 | 1.51E−11 | 3.56E−05 | 0 | 0.00035 |
| 676. | TNNC1 | NM_003280.1 | 1.302492998 | −0.34243175 | 4.20E−09 | 0.01679525 | 0 | 0.05887 |
| 677. | MCCC1 | NM_020166.3 | 1.302246373 | −0.12257597 | 4.12E−13 | 0.129876892 | 0 | 0.27791 |
| 678. | TAF13 | NM_005645.3 | 1.301676767 | 0.91664427 | 9.66E−11 | 3.40E−08 | 0 | 0 |
| 679. | TCEAL6 | NM_001006938.1 | 1.300207468 | 0.092987926 | 1.26E−11 | 0.331687907 | 0 | 0.52832 |
| 680. | CCNT1 | NM_001240.2 | 1.29946761 | 0.323578115 | 1.71E−11 | 0.002857177 | 0 | 0.01389 |
| 681. | THOC5 | NM_001002878.1 | 1.293641378 | 0.198067292 | 1.83E−12 | 0.028130358 | 0 | 0.08812 |
| 682. | DCLK2 | NM_001040260.1 | 1.292712666 | 0.001051025 | 7.33E−12 | 0.990814815 | 0 | 0.99641 |
| 683. | HIST2H2AA3 | NM_003516.2 | 1.29270342 | 1.068878593 | 6.00E−11 | 1.59E−09 | 0 | 0 |
| 684. | LOC732432 | XM_001724189.1 | 1.29255263 | 0.356110754 | 3.60E−13 | 0.000157208 | 0 | 0.0012 |
| 685. | LOC642414 | XR_016151.1 | 1.292393963 | 0.080639319 | 3.68E−12 | 0.363877307 | 0 | 0.55985 |
| 686. | LOC100128086 | XR_039419.1 | 1.290071693 | −0.18490951 | 6.51E−13 | 0.029470618 | 0 | 0.09135 |
| 687. | LOC650029 | XM_941861.1 | 1.288588402 | −0.15880653 | 8.82E−12 | 0.09585711 | 0 | 0.22333 |
| 688. | KLHL11 | NM_018143.1 | 1.28224461 | 0.481352064 | 1.15E−11 | 4.11E−05 | 0 | 0.00039 |
| 689. | CACYBP | NM_014412.2 | 1.281960322 | 0.100405054 | 9.02E−12 | 0.280273125 | 0 | 0.47356 |
| 690. | ZNF207 | NM_001032293.2 | 1.280488078 | −0.35384934 | 1.73E−11 | 0.00121518 | 0 | 0.00676 |
| 691. | MYH3 | NM_002470.2 | 1.279552852 | 0.040077269 | 1.23E−12 | 0.62562708 | 0 | 0.77824 |
| 692. | LOC100129186 | XM_001722466.1 | 1.276664758 | −0.06261277 | 1.17E−11 | 0.501268658 | 0 | 0.68228 |
| 693. | RAB8B | NM_016530.2 | 1.274043645 | −0.25001293 | 5.54E−11 | 0.020865118 | 0 | 0.06981 |
| 694. | UTP6 | NM_018428.2 | 1.273624089 | 0.077948838 | 3.39E−13 | 0.311461608 | 0 | 0.50699 |
| 695. | LOC727759 | XM_001125931.1 | 1.270342079 | −0.08623897 | 1.19E−10 | 0.415482102 | 0 | 0.60841 |
| 696. | C10orf137 | NM_015608.2 | 1.270304463 | −0.2203145 | 9.39E−11 | 0.043795827 | 0 | 0.1243 |
| 697. | LHX3 | NM_014564.2 | 1.269639702 | 0.181228781 | 1.56E−09 | 0.148067536 | 0 | 0.30529 |
| 698. | C17orf85 | NM_018553.1 | 1.267776498 | −0.34613961 | 1.81E−12 | 0.000426416 | 0 | 0.00282 |
| 699. | FAM90A6P | XR_016591.1 | 1.267761321 | −0.11675928 | 2.46E−11 | 0.231679429 | 0 | 0.41577 |
| 700. | IVNS1ABP | NM_006469.4 | 1.267243547 | −0.47174373 | 4.25E−12 | 2.34E−05 | 0 | 0.00024 |
| 701. | KIAA0831 | NM_014924.3 | 1.26652451 | −0.16734391 | 1.53E−12 | 0.05225327 | 0 | 0.1423 |
| 702. | PLS1 | NM_002670.1 | 1.266329601 | −0.17494654 | 1.31E−11 | 0.069705634 | 0 | 0.17675 |
| 703. | RBM15 | NM_022768.4 | 1.266035304 | 0.293252074 | 1.82E−11 | 0.00503893 | 0 | 0.02224 |
| 704. | ABCE1 | NM_001040876.1 | 1.262588568 | −0.26709225 | 2.32E−12 | 0.004241556 | 0 | 0.01934 |
| 705. | TMEM126A | NM_032273.2 | 1.261901695 | −0.05964487 | 2.33E−12 | 0.478764938 | 0 | 0.66392 |
| 706. | ITPR1 | NM_002222.4 | 1.261809954 | −0.0147413 | 1.48E−12 | 0.856699688 | 0 | 0.92516 |
| 707. | RLF | NM_012421.2 | 1.26115445 | 0.287736727 | 4.21E−12 | 0.003076025 | 0 | 0.01476 |
| 708. | UTP14A | NM_006649.2 | 1.257816378 | 0.095234161 | 1.32E−12 | 0.246855274 | 0 | 0.43415 |
| 709. | LGMN | NM_001008530.1 | 1.256081468 | 1.113926389 | 3.67E−10 | 2.80E−09 | 0 | 0 |
| 710. | RBBP5 | NM_005057.2 | 1.255816476 | 0.093609057 | 1.07E−11 | 0.308046571 | 0 | 0.5034 |
| 711. | CCDC109A | NM_138357.1 | 1.254274908 | −0.17212988 | 7.10E−12 | 0.062920858 | 0 | 0.16362 |
| 712. | LOC644863 | XM_927955.1 | 1.254068947 | 0.163338204 | 6.17E−12 | 0.074216318 | 0 | 0.18513 |
| 713. | EEF1B2 | NM_021121.2 | 1.248657387 | −0.12650563 | 5.85E−13 | 0.110892564 | 0 | 0.24841 |
| 714. | PTGR1 | NM_012212.2 | 1.248408075 | −0.07406433 | 1.00E−12 | 0.353941061 | 0 | 0.55047 |
| 715. | SLC4A7 | NM_003615.2 | 1.246394864 | −0.73843401 | 3.41E−11 | 1.97E−07 | 0 | 0 |
| 716. | LOC651441 | XM_940596.1 | 1.246244963 | 0.129210396 | 5.63E−11 | 0.200030139 | 0 | 0.37594 |
| 717. | LOC653544 | NM_001127388.1 | 1.245204137 | 8.133080049 | 5.52E−12 | 1.24E−27 | 0 | 0 |
| 718. | WDSOF1 | NM_015420.5 | 1.245112777 | 0.341385415 | 8.15E−12 | 0.000896331 | 0 | 0.00527 |
| 719. | LOC730740 | XM_001128558.1 | 1.245100265 | −0.50513988 | 2.33E−12 | 5.11E−06 | 0 | 7.00E−05 |
| 720. | MED30 | NM_080651.1 | 1.243913532 | −0.04610568 | 1.20E−12 | 0.563719573 | 0 | 0.73184 |
| 721. | FASTKD5 | NM_021826.4 | 1.243376527 | −0.04083647 | 5.19E−13 | 0.592075228 | 0 | 0.75304 |
| 722. | NFKBIB | NM_001001716.1 | 1.241142414 | 0.16150492 | 2.67E−11 | 0.098219921 | 0 | 0.22704 |
| 723. | TDRD3 | NM_030794.1 | 1.241044113 | 0.158162814 | 3.16E−10 | 0.155663666 | 0 | 0.31604 |
| 724. | EAF2 | NM_018456.4 | 1.240601106 | 0.006357779 | 2.61E−12 | 0.938766592 | 0 | 0.97063 |
| 725. | RBM24 | NM_153020.1 | 1.238467184 | −0.35037323 | 7.03E−12 | 0.000621695 | 0 | 0.00387 |
| 726. | LOC653884 | XM_936240.1 | 1.237831878 | 0.507599057 | 1.42E−11 | 1.62E−05 | 0 | 0.00018 |
| 727. | ZNF644 | NM_201269.1 | 1.237163651 | 0.1866136 | 8.64E−13 | 0.024652802 | 0 | 0.07977 |
| 728. | LOC146517 | XM_928464.1 | 1.23477277 | 0.12999852 | 8.73E−13 | 0.105411256 | 0 | 0.23913 |
| 729. | LTV1 | NM_032860.3 | 1.233857043 | 0.166110732 | 1.69E−12 | 0.049498263 | 0 | 0.13639 |
| 730. | SNRPB | NM_003091.3 | 1.233132452 | 0.057129747 | 1.02E−11 | 0.521802992 | 0 | 0.6983 |
| 731. | C17orf98 | NM_001080465.2 | 1.231755999 | −0.1664012 | 1.23E−08 | 0.223277849 | 0 | 0.40501 |
| 732. | TUFT1 | NM_020127.1 | 1.231104113 | −0.4862346 | 2.07E−11 | 3.32E−05 | 0 | 0.00033 |
| 733. | LOC728889 | XR_015885.2 | 1.230896717 | −0.31160961 | 1.89E−10 | 0.006845006 | 0 | 0.02868 |
| 734. | C10orf2 | NM_021830.3 | 1.229963691 | 0.212014111 | 1.82E−11 | 0.029394954 | 0 | 0.09118 |
| 735. | TPMT | NM_000367.2 | 1.229256548 | −0.82629722 | 2.47E−12 | 2.58E−09 | 0 | 0 |
| 736. | VGF | NM_003378.2 | 1.228290671 | 0.144420185 | 1.36E−11 | 0.119191485 | 0 | 0.2617 |
| 737. | LOC654121 | XM_942442.1 | 1.227259775 | 0.572587844 | 3.06E−11 | 5.35E−06 | 0 | 7.00E−05 |
| 738. | USP33 | NM_201624.1 | 1.227092095 | 0.364765018 | 3.21E−11 | 0.000864329 | 0 | 0.00511 |
| 739. | NLF2 | XM_940314.2 | 1.225068547 | −0.01440261 | 1.30E−08 | 0.914278599 | 0 | 0.95828 |
| 740. | EIF1AX | NM_001412.3 | 1.224893472 | 0.396862112 | 2.60E−08 | 0.009416068 | 0 | 0.0371 |
| 741. | DNAJB9 | NM_012328.1 | 1.224559886 | 0.297230165 | 7.88E−12 | 0.002486592 | 0 | 0.01233 |
| 742. | SCYL2 | NM_017988.4 | 1.223376659 | 0.47495511 | 6.42E−12 | 1.85E−05 | 0 | 2.00E−04 |
| 743. | TMED7 | NM_181836.3 | 1.223335413 | −0.04749956 | 2.79E−12 | 0.563673099 | 0 | 0.73182 |
| 744. | DSP | NM_001008844.1 | 1.223040013 | 0.42967214 | 1.91E−12 | 2.77E−05 | 0 | 0.00028 |
| 745. | TERF2IP | NM_018975.2 | 1.222689453 | −0.23536142 | 4.29E−13 | 0.004309204 | 0 | 0.01959 |
| 746. | U2AF2 | NM_007279.2 | 1.220559233 | 0.016936652 | 8.69E−13 | 0.825243869 | 0 | 0.9073 |
| 747. | U2AF1 | NM_001025203.1 | 1.219213779 | −0.041112 | 7.40E−12 | 0.634230121 | 0 | 0.7842 |
| 748. | SNORA63 | NR_002586.1 | 1.219018919 | −0.06620908 | 1.19E−11 | 0.457454961 | 0 | 0.64605 |
| 749. | HMGB2 | NM_002129.2 | 1.218723322 | −0.53026679 | 1.50E−07 | 0.002562451 | 0 | 0.01264 |
| 750. | DHX8 | NM_004941.1 | 1.218618843 | 0.210654498 | 5.11E−12 | 0.02013271 | 0 | 0.06787 |
| 751. | DOCK10 | NM_014689.2 | 1.218590663 | −0.042392 | 1.38E−12 | 0.59054411 | 0 | 0.75181 |
| 752. | LOC144438 | NR_024266.1 | 1.216625995 | 0.134450249 | 9.10E−12 | 0.133366661 | 0 | 0.28295 |
| 753. | WBP11 | NM_016312.2 | 1.215719368 | 0.398606804 | 2.78E−13 | 1.78E−05 | 0 | 0.00019 |
| 754. | PUM1 | NM_001020658.1 | 1.215582613 | 0.10228495 | 2.58E−12 | 0.215979086 | 0 | 0.39656 |
| 755. | PSMD12 | NM_002816.3 | 1.21295595 | 0.798891424 | 4.44E−12 | 6.17E−09 | 0 | 0 |
| 756. | CCDC148 | NM_138803.2 | 1.212800766 | 0.040306505 | 1.64E−11 | 0.653656949 | 0 | 0.79782 |
| 757. | ZCCHC8 | NM_017612.2 | 1.211248515 | 0.140658038 | 5.76E−12 | 0.107274482 | 0 | 0.2424 |
| 758. | DCP1A | NM_018403.4 | 1.210640832 | 0.495731651 | 2.96E−12 | 5.37E−06 | 0 | 7.00E−05 |
| 759. | ARL5B | NM_178815.3 | 1.210545187 | −0.09231272 | 1.11E−11 | 0.298109633 | 0 | 0.49291 |
| 760. | IMMP2L | NM_032549.2 | 1.209910969 | 0.118062245 | 2.12E−12 | 0.149914058 | 0 | 0.30778 |
| 761. | RFWD3 | NM_018124.3 | 1.208049138 | −0.11579668 | 1.10E−12 | 0.142978067 | 0 | 0.2976 |
| 762. | C20orf7 | NM_024120.3 | 1.206932447 | −0.02172869 | 1.68E−11 | 0.807832173 | 0 | 0.89708 |
| 763. | SCML2 | NM_006089.1 | 1.206323289 | −0.07204003 | 1.79E−11 | 0.424905947 | 0 | 0.6166 |
| 764. | FBXL20 | NM_032875.1 | 1.205956758 | 0.454990439 | 1.72E−10 | 0.000214189 | 0 | 0.00157 |
| 765. | LOC653541 | XM_927996.1 | 1.20581303 | 7.882897275 | 3.07E−11 | 7.96E−27 | 0 | 0 |
| 766. | DCP2 | NM_152624.4 | 1.204056822 | −0.3465364 | 2.71E−10 | 0.003144443 | 0 | 0.01504 |
| 767. | LOC388796 | NR_015366.2 | 1.20284251 | 0.039131411 | 7.49E−11 | 0.686674781 | 0 | 0.82025 |
| 768. | LOC652051 | XM_945171.1 | 1.201518638 | −0.07496917 | 2.53E−09 | 0.529864774 | 0 | 0.70492 |
| 769. | CYB5R4 | NM_016230.3 | 1.197397531 | 0.349078413 | 1.06E−09 | 0.005162324 | 0 | 0.0227 |
| 770. | NOMO3 | NM_001004067.2 | 1.195932458 | 0.046354378 | 1.14E−12 | 0.545034581 | 0 | 0.71717 |
| 771. | FRG2 | NM_001005217.1 | 1.193331745 | 0.021093081 | 5.12E−10 | 0.844224234 | 0 | 0.91796 |
| 772. | BCYRN1 | NR_001568.1 | 1.193135044 | −0.22422137 | 2.53E−11 | 0.020757969 | 0 | 0.06948 |
| 773. | LOC728732 | XR_015658.2 | 1.19197695 | −0.38091713 | 1.54E−09 | 0.003075392 | 0 | 0.01476 |
| 774. | RPS24 | NM_001026.3 | 1.191834275 | −0.02375199 | 1.55E−12 | 0.758786803 | 0 | 0.86777 |
| 775. | LOC729101 | XR_015731.1 | 1.191021556 | 0.08489762 | 1.84E−11 | 0.34338919 | 0 | 0.54043 |
| 776. | C16orf91 | NM_001010878.1 | 1.190020554 | 0.224307109 | 4.30E−12 | 0.011584594 | 0 | 0.0437 |
| 777. | FAM32A | NM_014077.2 | 1.189895447 | −0.36437445 | 5.59E−12 | 0.000251167 | 0 | 0.0018 |
| 778. | ARGLU1 | NM_018011.3 | 1.189872383 | −0.44077423 | 2.68E−12 | 1.81E−05 | 0 | 2.00E−04 |
| 779. | PDE12 | NM_177966.4 | 1.189087195 | −0.24628904 | 7.81E−12 | 0.007852276 | 0 | 0.0321 |
| 780. | NUDT1 | NM_198948.1 | 1.186929197 | −0.60529807 | 1.46E−11 | 8.85E−07 | 0 | 2.00E−05 |
| 781. | ZNF197 | NM_001024855.1 | 1.185963019 | 0.065216551 | 5.25E−09 | 0.595754477 | 0 | 0.75601 |
| 782. | RWDD1 | NM_016104.2 | 1.185820615 | 0.212048388 | 5.90E−11 | 0.033857374 | 0 | 0.10179 |
| 783. | GABPB2 | NM_016655.3 | 1.185669017 | 0.622815689 | 8.22E−11 | 2.29E−06 | 0 | 4.00E−05 |
| 784. | PPTC7 | NM_139283.1 | 1.18504186 | 0.377800479 | 1.22E−12 | 6.51E−05 | 0 | 0.00058 |
| 785. | C16orf33 | NM_024571.2 | 1.184672145 | −0.28869785 | 4.23E−13 | 0.000592512 | 0 | 0.00372 |
| 786. | EZH2 | NM_004456.3 | 1.18450651 | −0.09718009 | 1.20E−10 | 0.327044721 | 0 | 0.52385 |
| 787. | CLK3 | NM_003992.1 | 1.182797695 | 0.143529638 | 9.16E−11 | 0.147062855 | 0 | 0.30375 |
| 788. | C6orf211 | NM_024573.1 | 1.182525121 | −0.12028548 | 9.63E−12 | 0.166541987 | 0 | 0.33151 |
| 789. | PPHLN1 | NM_201438.1 | 1.180946148 | −0.0874507 | 4.44E−11 | 0.3487725 | 0 | 0.54521 |
| 790. | CCT2 | NM_006431.2 | 1.18058187 | −0.04058786 | 1.04E−11 | 0.634135173 | 0 | 0.7842 |
| 791. | RYK | NM_002958.3 | 1.180514518 | 0.034025682 | 7.51E−12 | 0.68423347 | 0 | 0.81863 |
| 792. | RNF38 | NM_022781.4 | 1.180119761 | −0.31966537 | 2.57E−12 | 0.000554487 | 0 | 0.00352 |
| 793. | FOSB | NM_006732.1 | 1.18009734 | 0.065219396 | 4.78E−11 | 0.483952007 | 0 | 0.66799 |
| 794. | 3-Mar | NM_178450.2 | 1.179405566 | −0.14780229 | 8.75E−12 | 0.090635754 | 0 | 0.2146 |
| 795. | F13A1 | NM_000129.3 | 1.179334053 | −0.22849384 | 4.00E−12 | 0.009520136 | 0 | 0.03736 |
| 796. | UTP23 | NM_032334.1 | 1.179003856 | 0.169136319 | 8.46E−11 | 0.088424102 | 0 | 0.21095 |
| 797. | ADAT3 | NM_138422.1 | 1.17865106 | 0.161462572 | 1.85E−12 | 0.046975442 | 0 | 0.13112 |
| 798. | IMPA1 | NM_005536.2 | 1.178227788 | −0.08075737 | 7.92E−10 | 0.46062656 | 0 | 0.64874 |
| 799. | RAE1 | NM_003610.3 | 1.178175402 | 0.092187439 | 2.08E−11 | 0.302608212 | 0 | 0.49776 |
| 800. | FKBP14 | NM_017946.2 | 1.177647758 | 0.034851434 | 9.59E−12 | 0.68041316 | 0 | 0.81551 |
| 801. | LOC653555 | XM_930357.1 | 1.177450046 | −0.10106816 | 1.38E−11 | 0.248889707 | 0 | 0.43674 |
| 802. | CHD2 | NM_001042572.2 | 1.177393315 | 0.536849393 | 1.23E−11 | 3.66E−06 | 0 | 5.00E−05 |
| 803. | LBH | XM_001132517.1 | 1.176505407 | 0.146755689 | 1.52E−10 | 0.147661982 | 0 | 0.30468 |
| 804. | LPAR2 | NM_004720.5 | 1.176329258 | −0.19958741 | 5.99E−10 | 0.073078115 | 0 | 0.18312 |
| 805. | UTX | NM_021140.1 | 1.175093332 | 0.368146383 | 1.77E−12 | 9.97E−05 | 0 | 0.00082 |
| 806. | C21orf66 | NM_013329.3 | 1.174366312 | 0.227324159 | 5.18E−12 | 0.010471937 | 0 | 0.04028 |
| 807. | PKN2 | NM_006256.2 | 1.173284488 | 0.376740622 | 7.94E−10 | 0.002174139 | 0 | 0.01104 |
| 808. | NCRNA00120 | NR_002767.1 | 1.173282961 | 0.154729511 | 3.69E−11 | 0.099773325 | 0 | 0.22967 |
| 809. | PAMR1 | NM_015430.2 | 1.172030741 | −0.53596645 | 6.27E−10 | 5.90E−05 | 0 | 0.00053 |
| 810. | HNRNPF | NM_001098204.1 | 1.171885264 | 0.04829931 | 9.03E−12 | 0.565705988 | 0 | 0.73349 |
| 811. | SESTD1 | NM_178123.3 | 1.171845532 | 0.559779016 | 1.09E−10 | 1.01E−05 | 0 | 0.00012 |
| 812. | KIAA1370 | NM_019600.1 | 1.170437676 | 0.222026045 | 5.61E−08 | 0.124466191 | 0 | 0.26955 |
| 813. | DUX5 | NM_012149.2 | 1.169110122 | 8.352310262 | 2.85E−11 | 1.29E−27 | 0 | 0 |
| 814. | H2AFX | NM_002105.2 | 1.168526313 | −0.40973461 | 9.73E−11 | 0.000324394 | 0 | 0.00224 |
| 815. | LOC151162 | NR_024275.1 | 1.168361058 | −0.52040882 | 1.72E−11 | 6.42E−06 | 0 | 8.00E−05 |
| 816. | FLNA | NM_001456.2 | 1.166988048 | −0.15565008 | 2.63E−08 | 0.250549307 | 0 | 0.43867 |
| 817. | KLC2 | XM_942434.1 | 1.166725495 | −0.05195465 | 3.31E−11 | 0.5639232 | 0 | 0.73198 |
| 818. | Cxorf40B | NM_001013845.1 | 1.165126358 | −0.20340904 | 1.58E−12 | 0.013176463 | 0 | 0.04859 |
| 819. | LOC649679 | XM_945045.1 | 1.164608362 | −0.10676387 | 4.20E−11 | 0.247388043 | 0 | 0.43485 |
| 820. | YTHDF3 | NM_152758.4 | 1.163867955 | −0.54389276 | 1.72E−10 | 1.85E−05 | 0 | 2.00E−04 |
| 821. | WDR1 | NM_017491.3 | 1.162128671 | 0.314399308 | 4.57E−12 | 0.000755102 | 0 | 0.00456 |
| 822. | GPR137C | NM_001099652.1 | 1.162065847 | −0.14626927 | 1.18E−11 | 0.094311309 | 0 | 0.22079 |
| 823. | ZNF280C | NM_017666.2 | 1.16196573 | −0.30812714 | 5.93E−11 | 0.003022016 | 0 | 0.01456 |
| 824. | SNRPA1 | NM_003090.2 | 1.160234865 | −0.0971834 | 6.59E−13 | 0.185745052 | 0 | 0.35735 |
| 825. | LOC731314 | XM_001129173.1 | 1.160171401 | −0.59088551 | 1.13E−11 | 7.32E−07 | 0 | 1.00E−05 |
| 826. | WDR33 | NM_001006623.1 | 1.159819745 | 0.034425292 | 1.90E−09 | 0.760249381 | 0 | 0.86846 |
| 827. | KIAA0174 | NM_014761.2 | 1.159746034 | 0.094593398 | 1.03E−11 | 0.264976548 | 0 | 0.45596 |
| 828. | CCNYL1 | NM_152523.1 | 1.158021572 | 0.324086783 | 4.91E−11 | 0.001799583 | 0 | 0.00944 |
| 829. | ZSCAN5A | NM_024303.1 | 1.157005262 | 0.099010624 | 2.61E−10 | 0.328308092 | 0 | 0.52502 |
| 830. | RQCD1 | NM_005444.1 | 1.156902459 | 0.284534409 | 3.03E−11 | 0.004009816 | 0 | 0.01845 |
| 831. | SYT14 | NM_153262.1 | 1.155984015 | −0.05094428 | 1.16E−09 | 0.641268115 | 0 | 0.78936 |
| 832. | ZFP37 | NM_003408.1 | 1.154571801 | −0.15669228 | 5.15E−12 | 0.061327996 | 0 | 0.16062 |
| 833. | LOC100132715 | XR_039129.1 | 1.153897859 | −0.59825112 | 1.23E−10 | 3.76E−06 | 0 | 5.00E−05 |
| 834. | ARPC5L | NM_030978.1 | 1.153323166 | 0.192210575 | 1.00E−11 | 0.02941857 | 0 | 0.09121 |
| 835. | EIF2C3 | NM_024852.2 | 1.153143186 | 0.088701042 | 5.10E−10 | 0.396584237 | 0 | 0.5905 |
| 836. | LOC648218 | XR_038470.1 | 1.15300723 | 0.116219282 | 5.04E−09 | 0.333733818 | 0 | 0.53075 |
| 837. | ERMP1 | NM_024896.2 | 1.152922839 | −0.34213368 | 1.83E−10 | 0.002063249 | 0 | 0.01058 |
| 838. | SLC25A26 | NM_173471.2 | 1.1527525 | 0.107031552 | 4.80E−11 | 0.245026441 | 0 | 0.43181 |
| 839. | LOC100134229 | NR_024451.1 | 1.151548683 | −0.07008176 | 1.95E−11 | 0.418539839 | 0 | 0.61073 |
| 840. | PDZD8 | NM_173791.3 | 1.150893598 | 0.690707165 | 9.87E−11 | 3.99E−07 | 0 | 1.00E−05 |
| 841. | CAMSAP1 | NM_015447.1 | 1.150184828 | −0.29416462 | 5.42E−11 | 0.003828029 | 0 | 0.01777 |
| 842. | GRPEL2 | NM_152407.3 | 1.149277281 | −0.34708288 | 3.43E−12 | 0.000219119 | 0 | 0.0016 |
| 843. | ATXN1L | NM_001137675.2 | 1.149100267 | 0.160679583 | 1.60E−11 | 0.069529635 | 0 | 0.17643 |
| 844. | JARID1A | NM_005056.1 | 1.148694966 | 0.660170132 | 3.60E−11 | 3.25E−07 | 0 | 1.00E−05 |
| 845. | HACL1 | NM_012260.2 | 1.148242322 | 0.170042244 | 5.42E−13 | 0.023782023 | 0 | 0.07753 |
| 846. | LOC652864 | XM_942571.1 | 1.147835016 | 0.251953952 | 3.58E−12 | 0.003853529 | 0 | 0.01786 |
| 847. | DNAJA2 | NM_005880.2 | 1.147267377 | −0.33265979 | 7.18E−12 | 0.000497232 | 0 | 0.00321 |
| 848. | NARG2 | NM_024611.4 | 1.146741454 | 0.176576894 | 3.88E−10 | 0.093477893 | 0 | 0.21943 |
| 849. | MRPL4 | NM_146387.1 | 1.145647285 | −0.02282466 | 1.17E−10 | 0.80945376 | 0 | 0.89811 |
| 850. | LOC402112 | XR_038697.1 | 1.144036715 | −0.3090144 | 2.74E−09 | 0.012338231 | 0 | 0.04605 |
| 851. | RBM25 | NM_021239.1 | 1.143184414 | −0.7474824 | 4.09E−10 | 3.77E−07 | 0 | 1.00E−05 |
| 852. | LOC440957 | NM_001124767.1 | 1.142658371 | −0.11333951 | 1.30E−11 | 0.183800864 | 0 | 0.35459 |
| 853. | RBM4 | NM_002896.2 | 1.142534032 | −0.12535865 | 3.80E−12 | 0.118666169 | 0 | 0.26092 |
| 854. | DOCK7 | NM_033407.2 | 1.141715883 | 0.120684314 | 2.54E−11 | 0.173016832 | 0 | 0.33974 |
| 855. | MTAP | NM_002451.3 | 1.141530828 | −0.42200093 | 4.84E−11 | 0.00012275 | 0 | 0.00098 |
| 856. | SPRY2 | NM_005842.2 | 1.141246432 | 0.116007546 | 1.52E−11 | 0.17724193 | 0 | 0.34566 |
| 857. | SGSH | NM_000199.2 | 1.141108849 | −0.07693406 | 2.86E−11 | 0.381155146 | 0 | 0.57683 |
| 858. | PSME4 | NM_014614.1 | 1.1409872 | −0.13684263 | 6.12E−12 | 0.098020161 | 0 | 0.2267 |
| 859. | SNRPD3 | NM_004175.3 | 1.140784492 | −0.40231415 | 1.57E−11 | 0.000102598 | 0 | 0.00084 |
| 860. | CDC20 | NM_001255.2 | 1.140182998 | −1.05610107 | 1.68E−11 | 6.52E−11 | 0 | 0 |
| 861. | TAPT1 | NM_153365.2 | 1.13931014 | −0.29519427 | 8.03E−12 | 0.0014642 | 0 | 0.00792 |
| 862. | IL34 | NM_152456.1 | 1.13658319 | −0.15420528 | 1.03E−11 | 0.071015728 | 0 | 0.17933 |
| 863. | KIF5C | NM_004522.1 | 1.136063506 | 0.284965136 | 2.62E−12 | 0.001132059 | 0 | 0.00638 |
| 864. | SNORD46 | NR_000024.2 | 1.135886497 | 0.126491002 | 7.94E−11 | 0.178079469 | 0 | 0.34639 |
| 865. | LOC650681 | XM_939769.1 | 1.135843324 | 0.252741513 | 1.16E−07 | 0.087843276 | 0 | 0.20994 |
| 866. | SDHD | NM_003002.1 | 1.135547623 | 0.16981842 | 6.16E−11 | 0.071811562 | 0 | 0.18078 |
| 867. | UTP3 | NM_020368.1 | 1.13507536 | −0.01605332 | 8.34E−10 | 0.878544114 | 0 | 0.93721 |
| 868. | SFRS13A | NM_054016.1 | 1.132010092 | 0.346810466 | 4.77E−12 | 0.000228251 | 0 | 0.00166 |
| 869. | RAPGEF6 | NM_016340.4 | 1.131041131 | −0.20229021 | 2.62E−10 | 0.049371237 | 0 | 0.13613 |
| 870. | BNIP2 | NM_004330.1 | 1.1307605 | 0.058327397 | 4.16E−11 | 0.509960162 | 0 | 0.68865 |
| 871. | LOC100129585 | XM_001720509.1 | 1.130482384 | −0.33768311 | 4.22E−11 | 0.000950757 | 0 | 0.00553 |
| 872. | ACTR6 | NM_022496.3 | 1.130266859 | 0.2078082 | 3.36E−10 | 0.046720379 | 0 | 0.13056 |
| 873. | SNORD3D | NR_006882.1 | 1.129349989 | −0.15047088 | 1.78E−09 | 0.178731436 | 0 | 0.34738 |
| 874. | PCDH7 | NM_002589.2 | 1.129103025 | −0.11725418 | 1.88E−11 | 0.173317449 | 0 | 0.34017 |
| 875. | ZFAND2A | NM_182491.1 | 1.127598591 | 0.424216292 | 1.71E−10 | 0.000219516 | 0 | 0.0016 |
| 876. | C18orf19 | NM_152352.1 | 1.125760245 | 0.576124294 | 9.33E−11 | 3.61E−06 | 0 | 5.00E−05 |
| 877. | CD55 | NM_000574.2 | 1.125583524 | 0.09757057 | 1.65E−08 | 0.437689181 | 0 | 0.62834 |
| 878. | ORC6L | NM_014321.2 | 1.124086663 | −0.25724434 | 1.59E−10 | 0.012257872 | 0 | 0.04578 |
| 879. | C9orf72 | NM_018325.1 | 1.123974845 | −0.05500311 | 1.48E−10 | 0.560325102 | 0 | 0.72912 |
| 880. | GART | NM_175085.1 | 1.121272949 | −0.0022074 | 1.25E−10 | 0.981050813 | 0 | 0.9915 |
| 881. | C2orf25 | NM_015702.1 | 1.120641663 | 0.203618336 | 2.79E−11 | 0.025460046 | 0 | 0.08178 |
| 882. | DNAJC12 | NM_201262.1 | 1.12040563 | −0.0362982 | 2.42E−10 | 0.707153619 | 0 | 0.83337 |
| 883. | USP38 | NM_032557.4 | 1.119769951 | −0.22060385 | 1.71E−11 | 0.014090426 | 0 | 0.05121 |
| 884. | C12orf43 | NM_022895.1 | 1.119308684 | 0.090747708 | 7.53E−11 | 0.320137982 | 0 | 0.51653 |
| 885. | KIAA1553 | XM_166320.6 | 1.118336577 | −0.16362906 | 3.17E−10 | 0.105928992 | 0 | 0.24001 |
| 886. | CCNE2 | NM_057735.1 | 1.118143253 | −0.54142828 | 2.03E−10 | 1.34E−05 | 0 | 0.00015 |
| 887. | LOC440013 | XM_495854.3 | 1.117710485 | −0.11283821 | 1.12E−09 | 0.290476588 | 0 | 0.48528 |
| 888. | HSPC111 | NM_016391.3 | 1.11521876 | −0.08294349 | 4.69E−11 | 0.348179771 | 0 | 0.54481 |
| 889. | CROP | NM_006107.2 | 1.115120822 | −0.02390027 | 1.42E−08 | 0.845309646 | 0 | 0.91816 |
| 890. | LOC650659 | XM_939743.1 | 1.114062456 | −0.018238 | 1.51E−10 | 0.845164054 | 0 | 0.91816 |
| 891. | TOP1P2 | NR_001283.1 | 1.113709756 | 0.457354204 | 1.29E−08 | 0.001124671 | 0 | 0.00635 |
| 892. | INA | NM_032727.2 | 1.113095856 | 0.371877537 | 1.20E−10 | 0.000585691 | 0 | 0.00368 |
| 893. | SNORD96A | NR_002592.1 | 1.111980201 | 0.173882795 | 7.81E−11 | 0.063957759 | 0 | 0.16557 |
| 894. | CTGF | NM_001901.1 | 1.108704726 | 0.048757922 | 1.47E−10 | 0.600387669 | 0 | 0.75992 |
| 895. | PELO | NM_015946.4 | 1.107903729 | 0.447764627 | 2.19E−11 | 2.61E−05 | 0 | 0.00027 |
| 896. | FAM13B | NM_001101800.1 | 1.107074212 | −0.39747766 | 2.19E−11 | 0.000103456 | 0 | 0.00084 |
| 897. | SECISBP2L | NM_014701.2 | 1.106578146 | −0.22306548 | 2.00E−10 | 0.026538534 | 0 | 0.0843 |
| 898. | PTRH2 | NM_016077.3 | 1.106198027 | 0.051646279 | 5.30E−10 | 0.60554211 | 0 | 0.76362 |
| 899. | ZNF326 | NM_182976.1 | 1.105462864 | 0.089833502 | 1.60E−09 | 0.402361041 | 0 | 0.59572 |
| 900. | MRPS22 | NM_020191.2 | 1.104391719 | 0.137510836 | 5.48E−12 | 0.084902738 | 0 | 0.20478 |
| 901. | ETFA | NM_000126.2 | 1.104011525 | −0.11194638 | 3.10E−12 | 0.143171588 | 0 | 0.29776 |
| 902. | UBE2C | NM_181800.1 | 1.103795231 | −0.66378591 | 1.29E−11 | 6.81E−08 | 0 | 0 |
| 903. | CPEB4 | NM_030627.1 | 1.103538487 | −0.04915988 | 6.54E−10 | 0.626317192 | 0 | 0.77881 |
| 904. | LRIG1 | NM_015541.2 | 1.103069438 | −0.3892228 | 9.87E−12 | 7.63E−05 | 0 | 0.00066 |
| 905. | MTERFD1 | NM_015942.3 | 1.101609269 | 0.203037686 | 8.82E−12 | 0.016698947 | 0 | 0.05858 |
| 906. | RNF11 | NM_014372.3 | 1.100921921 | 0.80070928 | 3.32E−12 | 9.01E−10 | 0 | 0 |
| 907. | API5 | NM_006595.2 | 1.100005738 | −0.35541321 | 7.13E−11 | 0.000606078 | 0 | 0.00379 |
| 908. | LOC100008589 | NR_003287.1 | 1.099836345 | 0.013221676 | 2.48E−11 | 0.87371699 | 0 | 0.93459 |
| 909. | TMEM41B | NM_015012.1 | 1.099587778 | −0.06296624 | 3.71E−12 | 0.404057125 | 0 | 0.59754 |
| 910. | DISCI | NM_018662.2 | 1.099547571 | 0.009834467 | 5.62E−09 | 0.931273035 | 0 | 0.96692 |
| 911. | POLR2C | NM_032940.2 | 1.099528324 | 0.144651532 | 2.39E−11 | 0.092968286 | 0 | 0.21857 |
| 912. | MELK | NM_014791.2 | 1.099204313 | 0.005893628 | 7.72E−11 | 0.946978282 | 0 | 0.97421 |
| 913. | CSPP1 | NM_024790.5 | 1.098156009 | −0.29171614 | 1.19E−11 | 0.001427133 | 0 | 0.00775 |
| 914. | ZFAND6 | NM_019006.2 | 1.098143704 | 0.121576691 | 5.23E−09 | 0.290628354 | 0 | 0.48546 |
| 915. | LOC645159 | XM_928195.2 | 1.097335884 | 0.343566046 | 2.37E−09 | 0.004355571 | 0 | 0.01976 |
| 916. | NUP35 | NM_138285.3 | 1.096524096 | 0.241203379 | 8.35E−12 | 0.005336103 | 0 | 0.02329 |
| 917. | C4orf32 | NM_152400.1 | 1.096395353 | −0.14449188 | 7.12E−11 | 0.112146071 | 0 | 0.25035 |
| 918. | TIPIN | NM_017858.1 | 1.096292861 | 0.336263475 | 1.87E−11 | 0.0004838 | 0 | 0.00315 |
| 919. | MTMR14 | NM_001077525.1 | 1.096198232 | −0.19881486 | 5.91E−08 | 0.142124407 | 0 | 0.2961 |
| 920. | AHSA1 | NM_012111.1 | 1.095960996 | 0.112980245 | 1.28E−10 | 0.223001167 | 0 | 0.40477 |
| 921. | FAM91A1 | NM_144963.2 | 1.095803947 | 1.249135004 | 5.30E−10 | 5.44E−11 | 0 | 0 |
| 922. | MTX3 | NM_001010891.3 | 1.095417954 | −0.06931404 | 5.25E−11 | 0.426238402 | 0 | 0.61793 |
| 923. | DYSF | NM_003494.2 | 1.095135096 | −0.70362085 | 7.27E−12 | 1.44E−08 | 0 | 0 |
| 924. | SDCBP | NM_001007067.1 | 1.094876133 | 0.74649087 | 1.94E−10 | 1.05E−07 | 0 | 0 |
| 925. | GOLGB1 | NM_004487.3 | 1.093155626 | 0.047716974 | 3.07E−11 | 0.570009169 | 0 | 0.73687 |
| 926. | TTC14 | NM_001042601.1 | 1.093029226 | 0.120133704 | 4.48E−09 | 0.289652805 | 0 | 0.48427 |
| 927. | LOC651959 | XM_941245.2 | 1.092888624 | −0.09423409 | 6.22E−11 | 0.286093964 | 0 | 0.48037 |
| 928. | DNTT | NM_001017520.1 | 1.09144065 | −0.0043337 | 5.52E−10 | 0.964932011 | 0 | 0.98418 |
| 929. | MATR3 | NM_199189.1 | 1.089506951 | 0.31286756 | 1.78E−09 | 0.007094557 | 0 | 0.02955 |
| 930. | FAM108B1 | NM_016014.2 | 1.089500547 | 0.279342324 | 1.19E−09 | 0.012538681 | 0 | 0.0467 |
| 931. | BMP4 | NM_130851.1 | 1.089143839 | 0.157561736 | 3.83E−11 | 0.073449004 | 0 | 0.1837 |
| 932. | RBP1 | NM_002899.2 | 1.08832098 | −0.50985444 | 2.41E−09 | 0.000110487 | 0 | 0.00089 |
| 933. | PDCL3 | NM_024065.3 | 1.088070063 | 0.022658241 | 6.04E−09 | 0.841613833 | 0 | 0.91659 |
| 934. | CBLL1 | NM_024814.1 | 1.088060901 | 0.833493724 | 3.99E−11 | 3.87E−09 | 0 | 0 |
| 935. | LOC100130856 | XM_001726438.1 | 1.086766448 | 0.072716194 | 2.91E−11 | 0.385084323 | 0 | 0.58059 |
| 936. | ALKBH1 | NM_006020.2 | 1.086751593 | 0.167891316 | 6.47E−12 | 0.037531806 | 0 | 0.1103 |
| 937. | LOC728643 | NR_003277.1 | 1.086411108 | −0.63353437 | 7.06E−11 | 4.59E−07 | 0 | 1.00E−05 |
| 938. | FLRT3 | NM_198391.1 | 1.085370713 | −0.50650589 | 5.46E−11 | 8.20E−06 | 0 | 1.00E−04 |
| 939. | PPP2CA | NM_002715.2 | 1.085290607 | −0.84059439 | 7.68E−12 | 6.81E−10 | 0 | 0 |
| 940. | KITLG | NM_000899.3 | 1.085131721 | −0.48718124 | 6.85E−11 | 1.58E−05 | 0 | 0.00018 |
| 941. | LOC729608 | XM_001714722.1 | 1.084391366 | 0.427810224 | 7.62E−11 | 7.94E−05 | 0 | 0.00068 |
| 942. | NHP2L1 | NM_001003796.1 | 1.083998244 | 0.280628365 | 1.74E−10 | 0.005678718 | 0 | 0.02454 |
| 943. | HIATL1 | NM_032558.2 | 1.083841715 | 0.127406235 | 1.27E−11 | 0.118068169 | 0 | 0.25999 |
| 944. | NRAS | NM_002524.2 | 1.081779536 | 0.507268201 | 4.41E−09 | 0.00016224 | 0 | 0.00124 |
| 945. | LCOR | NM_032440.1 | 1.081170798 | 0.153373454 | 9.61E−10 | 0.13951058 | 0 | 0.29203 |
| 946. | STIM2 | NM_020860.2 | 1.081169785 | 0.139486813 | 3.27E−10 | 0.15164717 | 0 | 0.31037 |
| 947. | C20orf4 | NM_015511.3 | 1.08024455 | 0.122711085 | 9.11E−12 | 0.123485859 | 0 | 0.26817 |
| 948. | TNNT2 | NM_001001431.1 | 1.078937179 | −1.30374985 | 7.68E−08 | 3.69E−09 | 0 | 0 |
| 949. | CDKN1A | NM_078467.1 | 1.078195342 | 0.61217478 | 1.54E−09 | 7.94E−06 | 0 | 1.00E−04 |
| 950. | FOS | NM_005252.2 | 1.077938553 | 0.471813546 | 5.00E−08 | 0.001353374 | 0 | 0.0074 |
| 951. | TRAPPC6B | NM_001079537.1 | 1.07714553 | 0.620681475 | 7.18E−10 | 3.59E−06 | 0 | 5.00E−05 |
| 952. | DIP2B | NM_173602.2 | 1.076215614 | −0.22003106 | 1.45E−09 | 0.042790054 | 0 | 0.12221 |
| 953. | LOC100128266 | XR_037888.1 | 1.075894157 | 0.090683001 | 5.06E−11 | 0.291263966 | 0 | 0.48606 |
| 954. | UBXN7 | NM_015562.1 | 1.075123071 | 0.303669543 | 2.10E−11 | 0.001102216 | 0 | 0.00624 |
| 955. | LOC649137 | XM_001131980.1 | 1.074657889 | 0.086009469 | 1.90E−11 | 0.289591841 | 0 | 0.48427 |
| 956. | PLAGL2 | NM_002657.2 | 1.074636945 | −0.10608532 | 1.23E−11 | 0.18446662 | 0 | 0.35553 |
| 957. | ENC1 | NM_003633.1 | 1.073105121 | −0.28244281 | 1.61E−10 | 0.00488697 | 0 | 0.0217 |
| 958. | CS | NM_004077.2 | 1.071166193 | −0.32349921 | 2.04E−11 | 0.000588363 | 0 | 0.0037 |
| 959. | TSC1 | NM_000368.3 | 1.070718233 | 0.132984568 | 2.53E−10 | 0.160748993 | 0 | 0.32322 |
| 960. | SNHG12 | NR_024127.1 | 1.069391677 | 0.173833734 | 1.61E−09 | 0.102695845 | 0 | 0.23446 |
| 961. | MAPRE3 | NM_012326.2 | 1.068880559 | 0.12586366 | 6.94E−11 | 0.152556734 | 0 | 0.31178 |
| 962. | ZNF509 | NM_145291.2 | 1.068683005 | 0.044510129 | 2.47E−10 | 0.629930314 | 0 | 0.78167 |
| 963. | NAF1 | NM_138386.1 | 1.068629462 | 0.14733421 | 8.56E−11 | 0.100810151 | 0 | 0.23132 |
| 964. | BRPF3 | NM_015695.2 | 1.066813315 | −0.0421277 | 1.19E−11 | 0.587612568 | 0 | 0.74952 |
| 965. | HDAC2 | NM_001527.2 | 1.066502454 | 0.013464456 | 1.00E−10 | 0.877414192 | 0 | 0.93679 |
| 966. | LOC652874 | XM_942590.1 | 1.066391837 | 0.005987456 | 2.58E−09 | 0.954742823 | 0 | 0.9788 |
| 967. | ZMYM5 | NM_001039650.1 | 1.0652354 | 0.06857313 | 8.66E−11 | 0.431333338 | 0 | 0.6223 |
| 968. | GALM | NM_138801.1 | 1.06343722 | 0.000591631 | 8.74E−12 | 0.993776069 | 0 | 0.99739 |
| 969. | UPF2 | NM_080599.1 | 1.063309533 | 0.174018826 | 8.51E−10 | 0.088977545 | 0 | 0.21178 |
| 970. | ZFP91 | NM_053023.3 | 1.061621244 | 0.533426555 | 3.95E−09 | 7.03E−05 | 0 | 0.00061 |
| 971. | NUP153 | NM_005124.2 | 1.059886645 | −0.07908733 | 1.79E−10 | 0.382448966 | 0 | 0.57803 |
| 972. | PRPF3 | NM_004698.1 | 1.058354904 | 0.122657814 | 3.09E−11 | 0.141136078 | 0 | 0.2945 |
| 973. | LOC646786 | XM_929738.1 | 1.0577539 | 0.092682481 | 1.41E−09 | 0.363633182 | 0 | 0.5596 |
| 974. | PRPF38A | NM_032864.3 | 1.057666912 | −0.17014044 | 8.89E−10 | 0.094963043 | 0 | 0.22176 |
| 975. | LOC647081 | XR_017490.2 | 1.057295145 | 0.08645715 | 1.22E−10 | 0.329087945 | 0 | 0.52586 |
| 976. | LOC643509 | XM_932666.2 | 1.057259344 | 0.004510543 | 1.11E−08 | 0.96851601 | 0 | 0.98597 |
| 977. | RN7SK | NR_001445.1 | 1.055974111 | 0.467639661 | 3.45E−08 | 0.00099303 | 0 | 0.00573 |
| 978. | LOC641844 | XR_018036.2 | 1.055485991 | 0.034857969 | 4.64E−11 | 0.673977402 | 0 | 0.81163 |
| 979. | LOC729120 | XM_001133026.1 | 1.054623336 | −0.01773804 | 1.03E−10 | 0.837515158 | 0 | 0.9142 |
| 980. | KHSRP | NM_003685.2 | 1.053086053 | 0.175919484 | 2.53E−10 | 0.064293983 | 0 | 0.16625 |
| 981. | LOC645691 | XM_928701.3 | 1.052836096 | −0.43820017 | 3.08E−08 | 0.001599259 | 0 | 0.00855 |
| 982. | ZFYVE1 | NM_021260.1 | 1.051285604 | 0.133970068 | 5.62E−11 | 0.119019359 | 0 | 0.26142 |
| 983. | SYAP1 | NM_032796.2 | 1.050864137 | 0.498717647 | 4.36E−09 | 0.000141491 | 0 | 0.0011 |
| 984. | LOC644877 | XR_017355.2 | 1.050610394 | 0.090333661 | 4.83E−11 | 0.280687722 | 0 | 0.47387 |
| 985. | LOC647037 | XM_930029.1 | 1.050461424 | 0.115257018 | 6.54E−11 | 0.179670227 | 0 | 0.34878 |
| 986. | FAM103A1 | NM_031452.2 | 1.049593448 | 0.548964175 | 1.41E−09 | 2.10E−05 | 0 | 0.00022 |
| 987. | FLRT2 | NM_013231.4 | 1.048015868 | −0.5218431 | 1.47E−10 | 7.35E−06 | 0 | 9.00E−05 |
| 988. | NUP155 | NM_153485.1 | 1.047710989 | 0.074659225 | 2.47E−10 | 0.412428516 | 0 | 0.60612 |
| 989. | SLC25A38 | NM_017875.1 | 1.047432233 | −0.01502885 | 3.29E−11 | 0.851906405 | 0 | 0.92211 |
| 990. | SNRPB2 | NM_198220.1 | 1.046529006 | −0.01035738 | 8.35E−11 | 0.902747873 | 0 | 0.95146 |
| 991. | AZIN1 | NM_015878.4 | 1.046116284 | 0.275367926 | 7.12E−10 | 0.008801689 | 0 | 0.03517 |
| 992. | LOC729920 | NM_001101426.2 | 1.044490551 | 0.182331537 | 8.67E−12 | 0.022336598 | 0 | 0.07357 |
| 993. | FAM176A | NM_001135032.1 | 1.043068124 | 0.679565693 | 9.32E−11 | 1.11E−07 | 0 | 0 |
| 994. | BTBD7 | NM_018167.3 | 1.042663787 | 0.076298792 | 4.05E−10 | 0.413418603 | 0 | 0.60671 |
| 995. | DIRC2 | NM_032839.1 | 1.042199441 | 0.33518563 | 3.81E−11 | 0.000450503 | 0 | 0.00295 |
| 996. | LOC642268 | XM_930669.1 | 1.042113396 | 0.093744876 | 4.75E−08 | 0.450937945 | 0 | 0.64012 |
| 997. | PRICKLE1 | NM_153026.1 | 1.0419728 | −0.32716061 | 8.88E−10 | 0.002741031 | 0 | 0.01338 |
| 998. | RSL1D1 | NM_015659.2 | 1.041324635 | 0.198372858 | 2.86E−11 | 0.020013413 | 0 | 0.06755 |
| 999. | LOC647150 | XR_017449.2 | 1.041135995 | 0.401698852 | 2.58E−10 | 0.000216086 | 0 | 0.00158 |
| 1000. | HK2 | NM_000189.4 | 1.040582509 | 0.756005024 | 6.67E−12 | 1.76E−09 | 0 | 0 |
| 1001. | ZNF280B | NM_080764.2 | 1.040377213 | −0.34290809 | 1.03E−10 | 0.000612546 | 0 | 0.00382 |
| 1002. | LOC648210 | XR_018923.1 | 1.040288367 | −0.54383326 | 6.96E−08 | 0.000313357 | 0 | 0.00217 |
| 1003. | RECQL | NM_032941.1 | 1.040035529 | 0.682791422 | 4.19E−11 | 4.86E−08 | 0 | 0 |
| 1004. | C7orf40 | NR_003697.1 | 1.039297922 | 0.254352266 | 1.25E−08 | 0.034232718 | 0 | 0.10265 |
| 1005. | PABPC4L | NM_001114734.1 | 1.039177821 | −0.28475758 | 2.21E−10 | 0.004165504 | 0 | 0.01904 |
| 1006. | RIF1 | NM_018151.3 | 1.038795437 | 0.25544696 | 2.27E−10 | 0.008885581 | 0 | 0.0354 |
| 1007. | MYC | NM_002467.3 | 1.037900386 | 0.3695377 | 3.03E−11 | 0.000138606 | 0 | 0.00108 |
| 1008. | LOC652481 | XM_941942.1 | 1.037532026 | 0.112115526 | 1.14E−09 | 0.259332229 | 0 | 0.44874 |
| 1009. | ARMC5 | NM_024742.1 | 1.037457586 | 0.079707127 | 3.61E−10 | 0.387807213 | 0 | 0.58317 |
| 1010. | C4orf39 | NM_153027.1 | 1.037314675 | −0.01489551 | 2.43E−10 | 0.867606731 | 0 | 0.93093 |
| 1011. | LRRC42 | NM_052940.3 | 1.035624329 | −0.04027074 | 8.03E−11 | 0.631371597 | 0 | 0.78245 |
| 1012. | SLC25A25 | NM_052901.2 | 1.035390695 | −0.43420388 | 4.21E−11 | 2.61E−05 | 0 | 0.00027 |
| 1013. | CIRH1A | NM_032830.1 | 1.034638884 | 0.162219004 | 1.91E−11 | 0.045901428 | 0 | 0.12876 |
| 1014. | NOMO1 | NM_014287.3 | 1.034361189 | 0.020524855 | 1.37E−08 | 0.856292177 | 0 | 0.92479 |
| 1015. | LOC649167 | XM_938236.1 | 1.03294322 | −0.00095784 | 1.72E−08 | 0.993340796 | 0 | 0.99732 |
| 1016. | UTP11L | NM_016037.2 | 1.03253166 | 0.126861343 | 1.01E−10 | 0.144427742 | 0 | 0.29971 |
| 1017. | FAM126B | NM_173822.2 | 1.03203744 | 0.210382913 | 3.07E−11 | 0.013978022 | 0 | 0.05093 |
| 1018. | OTUD6B | NM_016023.2 | 1.03061043 | −0.25011699 | 2.20E−10 | 0.009555644 | 0 | 0.03747 |
| 1019. | OCIAD2 | NM_152398.2 | 1.030571051 | 0.27354447 | 1.58E−09 | 0.011237412 | 0 | 0.04266 |
| 1020. | OVOL1 | XM_001129344.1 | 1.029828388 | −0.05407431 | 1.62E−10 | 0.534543893 | 0 | 0.70883 |
| 1021. | MAK10 | NM_024635.3 | 1.029561675 | 0.263536187 | 1.23E−10 | 0.005344587 | 0 | 0.02332 |
| 1022. | C12orf35 | NM_018169.2 | 1.029288164 | 0.138049248 | 1.28E−09 | 0.168036349 | 0 | 0.33326 |
| 1023. | TROVE2 | NM_001042370.1 | 1.028979236 | 0.169282342 | 5.63E−10 | 0.080488255 | 0 | 0.197 |
| 1024. | LOC648638 | XM_937706.1 | 1.028567866 | 0.223742233 | 2.87E−11 | 0.009165968 | 0 | 0.03628 |
| 1025. | LOC642812 | XR_036892.1 | 1.028443066 | 0.256454233 | 2.83E−10 | 0.008820126 | 0 | 0.03521 |
| 1026. | HNRNPAB | NM_031266.2 | 1.026350617 | −0.24070782 | 7.90E−11 | 0.008160613 | 0 | 0.03309 |
| 1027. | LOC643167 | XR_038497.1 | 1.026257842 | 0.228448193 | 3.03E−11 | 0.008047914 | 0 | 0.03272 |
| 1028. | HIST1H4H | NM_003543.3 | 1.025732011 | 1.2313351 | 3.41E−10 | 1.38E−11 | 0 | 0 |
| 1029. | YTHDC1 | NM_001031732.2 | 1.025117772 | 0.182849261 | 2.04E−10 | 0.04700437 | 0 | 0.13117 |
| 1030. | CD2AP | NM_012120.2 | 1.025051265 | −0.01299116 | 6.14E−10 | 0.889108296 | 0 | 0.94308 |
| 1031. | C16orf61 | NM_020188.2 | 1.024443103 | −0.25843268 | 7.05E−12 | 0.001726012 | 0 | 0.00912 |
| 1032. | ZMAT2 | NM_144723.1 | 1.024373429 | 0.172328577 | 7.71E−12 | 0.026128565 | 0 | 0.08343 |
| 1033. | C12orf31 | NM_032338.2 | 1.024231622 | −0.0757358 | 8.37E−10 | 0.428059074 | 0 | 0.61989 |
| 1034. | GTF2A2 | NM_004492.1 | 1.02237211 | −0.198989 | 1.42E−10 | 0.028671893 | 0 | 0.08945 |
| 1035. | LOC100132528 | XR_038720.1 | 1.022350592 | −0.74065639 | 4.22E−10 | 8.24E−08 | 0 | 0 |
| 1036. | TOMM40 | NM_006114.1 | 1.022228207 | −0.08335431 | 4.55E−11 | 0.303971739 | 0 | 0.49905 |
| 1037. | YWHAE | NM_006761.3 | 1.021995926 | −0.09076355 | 4.35E−10 | 0.324800606 | 0 | 0.52151 |
| 1038. | SNAPC2 | NM_003083.2 | 1.021301486 | −0.05465569 | 1.50E−09 | 0.577920709 | 0 | 0.74199 |
| 1039. | TRO | NM_001039705.1 | 1.021058495 | −0.22973069 | 2.00E−11 | 0.006408205 | 0 | 0.02713 |
| 1040. | STIP1 | NM_006819.1 | 1.019923878 | −0.12480443 | 2.66E−11 | 0.118431632 | 0 | 0.26054 |
| 1041. | GOLPH4 | NM_014498.2 | 1.018008616 | 0.715057288 | 1.96E−10 | 6.70E−08 | 0 | 0 |
| 1042. | URB2 | NM_014777.2 | 1.0167874 | −0.28830611 | 1.06E−11 | 0.000752289 | 0 | 0.00455 |
| 1043. | TRK1 | NR_001449.1 | 1.016402194 | 0.06997919 | 1.82E−09 | 0.480194904 | 0 | 0.66512 |
| 1044. | ZDHHC14 | NM_024630.2 | 1.016284079 | 0.084770694 | 9.91E−09 | 0.440766133 | 0 | 0.63111 |
| 1045. | FAM90A18 | XM_496955.4 | 1.015859899 | 0.065543358 | 2.28E−09 | 0.513587831 | 0 | 0.69157 |
| 1046. | LOC100130550 | XR_037892.1 | 1.015722289 | 0.533948234 | 1.71E−09 | 2.27E−05 | 0 | 0.00024 |
| 1047. | LOC730153 | XM_001717676.1 | 1.015289849 | −0.00504612 | 3.32E−11 | 0.948440233 | 0 | 0.97509 |
| 1048. | HNRNPA1 | NM_031157.2 | 1.014750875 | −0.42094853 | 2.92E−11 | 2.32E−05 | 0 | 0.00024 |
| 1049. | COX10 | NM_001303.2 | 1.014240958 | −0.05259565 | 5.17E−11 | 0.512849102 | 0 | 0.69087 |
| 1050. | LOC728022 | XM_001720082.1 | 1.014197516 | −0.20136281 | 7.68E−09 | 0.07187497 | 0 | 0.18088 |
| 1051. | CENPN | NM_018455.3 | 1.013635291 | 0.001233995 | 2.88E−11 | 0.987260072 | 0 | 0.99438 |
| 1052. | CTDSPL2 | NM_016396.2 | 1.013554506 | 0.353555626 | 2.36E−10 | 0.000562438 | 0 | 0.00356 |
| 1053. | TRMT11 | NM_001031712.2 | 1.01294925 | −0.06262036 | 2.60E−11 | 0.418764287 | 0 | 0.61097 |
| 1054. | RPF2 | NM_032194.1 | 1.011426861 | 0.492183836 | 2.84E−10 | 1.60E−05 | 0 | 0.00018 |
| 1055. | SLC25A36 | NM_018155.1 | 1.009418195 | 0.308796337 | 1.94E−09 | 0.004729358 | 0 | 0.02114 |
| 1056. | DPM1 | NM_003859.1 | 1.008124946 | 0.013439446 | 2.82E−10 | 0.878048978 | 0 | 0.93691 |
| 1057. | RNU6-15 | NR_028372.1 | 1.00785758 | 0.710506662 | 1.87E−10 | 6.07E−08 | 0 | 0 |
| 1058. | LOC100133950 | XM_001721634.1 | 1.007465188 | −0.14119302 | 1.60E−08 | 0.214330739 | 0 | 0.3943 |
| 1059. | U2AF1L2 | NM_005089.1 | 1.00671488 | 0.144171307 | 8.07E−11 | 0.088145085 | 0 | 0.21044 |
| 1060. | LOC651864 | XM_944981.1 | 1.006372187 | 0.013469472 | 4.00E−09 | 0.895197227 | 0 | 0.94682 |
| 1061. | SSR2 | XM_945430.1 | 1.006216181 | 0.009945239 | 4.38E−10 | 0.911682417 | 0 | 0.95632 |
| 1062. | SFT2D2 | NM_199344.1 | 1.005497503 | 0.367781793 | 1.58E−10 | 0.000279846 | 0 | 0.00197 |
| 1063. | BCL2L11 | NM_006538.3 | 1.005369308 | 0.161087859 | 3.24E−09 | 0.121645079 | 0 | 0.26556 |
| 1064. | ILF3 | NM_004516.2 | 1.004527624 | −0.20420779 | 1.11E−10 | 0.02135674 | 0 | 0.07109 |
| 1065. | FAM90A15 | XM_001726945.1 | 1.004342636 | 0.009238588 | 6.36E−10 | 0.919542528 | 0 | 0.96147 |
| 1066. | GADD45B | NM_015675.2 | 1.003484506 | 0.350422477 | 1.44E−10 | 0.00042227 | 0 | 0.0028 |
| 1067. | PSPC1 | NM_001042414.1 | 1.002965925 | −0.24270314 | 1.31E−08 | 0.036522764 | 0 | 0.10799 |
| 1068. | IDI1 | NM_004508.2 | 1.002827597 | −0.21356264 | 1.19E−09 | 0.033719799 | 0 | 0.10147 |
| 1069. | TAF9 | NM_001015891.1 | 1.00218608 | 0.314494729 | 3.89E−09 | 0.005311051 | 0 | 0.0232 |
| 1070. | DUSP6 | NM_022652.2 | 1.00169102 | 0.274501313 | 1.07E−09 | 0.00790461 | 0 | 0.03227 |
| 1071. | RBM28 | NM_018077.1 | 1.001253323 | 0.371778522 | 5.49E−10 | 0.000493853 | 0 | 0.0032 |
| 1072. | ACTR5 | NM_024855.3 | 1.001084095 | −0.17674663 | 2.39E−10 | 0.050990782 | 0 | 0.13957 |
| 1073. | STX11 | NM_003764.2 | 0.9255359 | 1.156607294 | 2.71E−10 | 5.25E−12 | 0 | 0 |
| 1074. | RORA | NM_002943.2 | 0.892828404 | 1.888665695 | 2.31E−09 | 3.17E−15 | 0 | 0 |
| 1075. | YME1L1 | NM_139313.1 | 0.885338942 | 1.012476977 | 7.74E−10 | 7.60E−11 | 0 | 0 |
| 1076. | DNAJC3 | NM_006260.2 | 0.866723986 | 1.604460862 | 1.04E−07 | 3.07E−12 | 0 | 0 |
| 1077. | RDH5 | NM_002905.2 | 0.86041216 | −1.19985636 | 6.03E−09 | 2.00E−11 | 0 | 0 |
| 1078. | DUX3 | NM_012148.2 | 0.832695437 | 7.831652893 | 3.05E−09 | 1.20E−27 | 0 | 0 |
| 1079. | MGC87042 | XM_001128032.1 | 0.822066529 | 1.301716471 | 8.12E−08 | 3.78E−11 | 0 | 0 |
| 1080. | AKIRIN1 | NM_024595.1 | 0.817666967 | 1.027987047 | 1.72E−09 | 3.30E−11 | 0 | 0 |
| 1081. | ACTG2 | NM_001615.3 | 0.611516577 | −1.02765041 | 9.24E−06 | 4.10E−09 | 3.00E−05 | 0 |
| 1082. | OGFRL1 | NM_024576.3 | 0.600178387 | 1.17123344 | 2.66E−05 | 1.39E−09 | 9.00E−05 | 0 |
| 1083. | NRP1 | NM_001024629.1 | 0.578245006 | 1.051207308 | 1.19E−06 | 8.05E−11 | 1.00E−05 | 0 |
| 1084. | CDKN1C | NM_000076.1 | 0.570682201 | −1.05155747 | 1.43E−06 | 8.01E−11 | 1.00E−05 | 0 |
| 1085. | STEAP1 | NM_012449.2 | 0.545080861 | 1.029472388 | 1.73E−06 | 6.59E−11 | 1.00E−05 | 0 |
| 1086. | UBE2N | NM_003348.3 | 0.524162096 | −1.07963714 | 4.18E−06 | 4.47E−11 | 2.00E−05 | 0 |
| 1087. | ACO1 | NM_002197.1 | 0.52007481 | −1.15783196 | 4.58E−05 | 3.17E−10 | 0.00015 | 0 |
| 1088. | CLDN5 | NM_003277.2 | 0.502369548 | −1.37078899 | 1.23E−05 | 1.26E−12 | 4.00E−05 | 0 |
| 1089. | CGGBP1 | NM_003663.3 | 0.462779712 | 1.045033157 | 4.44E−05 | 2.38E−10 | 0.00014 | 0 |
| 1090. | LOC441455 | XR_041340.1 | 0.441963221 | 1.002721498 | 0.000350086 | 5.42E−09 | 0.00095 | 0 |
| 1091. | LOC651861 | XM_001719902.1 | 0.393308176 | 6.597749965 | 0.001275915 | 3.94E−24 | 0.0031 | 0 |
| 1092. | MCL1 | NM_021960.3 | 0.383311489 | 1.672724945 | 0.000244946 | 2.57E−14 | 0.00068 | 0 |
| 1093. | HNRNPAO | NM_006805.3 | 0.345768683 | −1.25021419 | 0.000437807 | 2.41E−12 | 0.00116 | 0 |
| 1094. | LOC203547 | NM_001017980.2 | 0.342264638 | −1.09033514 | 0.001022675 | 1.17E−10 | 0.00253 | 0 |
| 1095. | AK3L1 | NM_203464.1 | 0.328543021 | 1.026597459 | 0.001018104 | 1.62E−10 | 0.00252 | 0 |
| 1096. | ABL2 | NM_007314.2 | 0.316857819 | 1.066692333 | 0.002098229 | 1.93E−10 | 0.00487 | 0 |
| 1097. | HN1 | NM_016185.2 | 0.307197324 | −1.03615031 | 0.001150568 | 5.40E−11 | 0.00282 | 0 |
| 1098. | SFRS6 | NM_006275.4 | 0.307168529 | −1.01210641 | 0.000906724 | 5.10E−11 | 0.00227 | 0 |
| 1099. | HIST1H2AC | NM_003512.3 | 0.260774576 | 1.066326492 | 0.005750248 | 6.64E−11 | 0.01218 | 0 |
| 1100. | SOX8 | NM_014587.2 | 0.169095918 | −1.26116236 | 0.053417055 | 2.20E−12 | 0.09055 | 0 |
| 1101. | ARFGEF2 | NM_006420.2 | 0.125067472 | 1.336273378 | 0.224568998 | 2.47E−11 | 0.31242 | 0 |
| 1102. | TIMM23B | XM_928114.3 | 0.115549462 | 1.879844743 | 0.359592652 | 2.38E−12 | 0.45772 | 0 |
| 1103. | LOC100133997 | XM_001715556.1 | 0.111353621 | 2.784961 | 0.223354649 | 2.89E−18 | 0.31098 | 0 |
| 1104. | CCL20 | NM_004591.1 | 0.109855803 | 1.630861801 | 0.282130945 | 6.26E−13 | 0.37593 | 0 |
| 1105. | ELK4 | NM_001973.2 | 0.109492959 | 1.305687487 | 0.305027251 | 7.75E−11 | 0.4008 | 0 |
| 1106. | WDR36 | NM_139281.2 | 0.09600657 | −1.06945542 | 0.221989934 | 1.06E−11 | 0.30948 | 0 |
| 1107. | SPCS3 | NM_021928.2 | 0.09520259 | 1.036524317 | 0.235186656 | 2.70E−11 | 0.32466 | 0 |
| 1108. | ABHD2 | NM_152924.3 | 0.091334877 | 1.047075939 | 0.388131846 | 3.13E−09 | 0.48657 | 0 |
| 1109. | ALOX5AP | NM_001629.2 | 0.085945199 | −1.02892181 | 0.354225084 | 4.43E−10 | 0.45226 | 0 |
| 1110. | FGFR4 | NM_213647.1 | 0.082598045 | −1.05368209 | 0.404016027 | 9.26E−10 | 0.5024 | 0 |
| 1111. | PAFAH1B2 | NM_002572.2 | 0.062231978 | 1.073521452 | 0.485847616 | 1.19E−10 | 0.58111 | 0 |
| 1112. | SHD | NM_020209.2 | 0.058233212 | −1.12342048 | 0.486197866 | 1.68E−11 | 0.5814 | 0 |
| 1113. | AIM2 | NM_004833.1 | 0.038083639 | 1.074477056 | 0.775655702 | 9.97E−08 | 0.83218 | 0 |
| 1114. | CD274 | NM_014143.2 | 0.032275451 | 1.364691343 | 0.736102256 | 6.36E−12 | 0.79956 | 0 |
| 1115. | FBXO6 | NM_018438.4 | 0.031481072 | 1.017628884 | 0.740715281 | 9.71E−10 | 0.8033 | 0 |
| 1116. | DUX2 | NM_012147.2 | 0.030665701 | 1.029618827 | 0.716259583 | 9.97E−11 | 0.78326 | 0 |
| 1117. | RET | NM_020975.4 | 0.024106598 | 1.110768839 | 0.787715983 | 7.55E−11 | 0.84159 | 0 |
| 1118. | CUL4B | NM_001079872.1 | 0.023471479 | 1.098940558 | 0.811846314 | 4.88E−10 | 0.86066 | 0 |
| 1119. | C10orf6 | NM_018121.2 | 0.000177537 | −1.17382939 | 0.998280312 | 6.53E−12 | 0.99863 | 0 |
| 1120. | LOC652641 | XM_942195.1 | −0.00864993 | 1.077990314 | 0.949506215 | 1.35E−07 | 0.96253 | 0 |
| 1121. | UBE2L3 | NM_198157.1 | −0.01341915 | −1.09204802 | 0.867605419 | 1.57E−11 | 0.90231 | 0 |
| 1122. | RALA | NM_005402.2 | −0.03700305 | −1.21157879 | 0.657272573 | 4.40E−12 | 0.73323 | 0 |
| 1123. | OLFML2B | NM_015441.1 | −0.04541052 | −1.28414302 | 0.636267293 | 1.93E−11 | 0.71543 | 0 |
| 1124. | SFRS2IP | NM_004719.2 | −0.05649792 | −1.06811987 | 0.51966034 | 9.77E−11 | 0.61198 | 0 |
| 1125. | STK17B | NM_004226.2 | −0.05995437 | 1.309143946 | 0.542561363 | 2.07E−11 | 0.63341 | 0 |
| 1126. | CYP2J2 | NM_000775.2 | −0.06947768 | 1.127417514 | 0.504412013 | 7.28E−10 | 0.59783 | 0 |
| 1127. | UBR1 | NM_174916.1 | −0.08618191 | 1.111143825 | 0.33001699 | 4.99E−11 | 0.42689 | 0 |
| 1128. | USP13 | NM_003940.2 | −0.08799215 | −1.03814996 | 0.407571152 | 3.90E−09 | 0.50579 | 0 |
| 1129. | NES | NM_006617.1 | −0.08904184 | −1.1178013 | 0.287165139 | 1.59E−11 | 0.38115 | 0 |
| 1130. | SERPINB8 | NM_198833.1 | −0.10463003 | 1.016402892 | 0.277407814 | 9.66E−10 | 0.37084 | 0 |
| 1131. | PANX1 | NM_015368.3 | −0.11404982 | 1.232833893 | 0.222933166 | 1.88E−11 | 0.31052 | 0 |
| 1132. | PPM1K | NM_152542.2 | −0.14597856 | 1.440041018 | 0.05468873 | 1.39E−14 | 0.09244 | 0 |
| 1133. | HSPA13 | NM_006948.4 | −0.17560967 | 1.339950446 | 0.066870745 | 4.13E−12 | 0.11054 | 0 |
| 1134. | LOC649425 | XM_938508.1 | −0.20651231 | 2.538886962 | 0.042102553 | 6.55E−17 | 0.07337 | 0 |
| 1135. | UHRF1 | NM_001048201.1 | −0.20908283 | −1.03401173 | 0.048115314 | 1.91E−09 | 0.0826 | 0 |
| 1136. | RUNX1 | NM_001754.3 | −0.21076513 | −1.0466384 | 0.059906433 | 4.38E−09 | 0.10021 | 0 |
| 1137. | LOC730996 | XM_001128017.1 | −0.2204346 | 1.048730809 | 0.012172606 | 3.50E−11 | 0.02403 | 0 |
| 1138. | STRN | NM_003162.2 | −0.23478993 | 1.092059428 | 0.046030603 | 4.45E−09 | 0.07943 | 0 |
| 1139. | AASDHPPT | NM_015423.2 | −0.23796404 | −1.24578049 | 0.052732631 | 1.06E−09 | 0.08952 | 0 |
| 1140. | SERBP1 | NM_001018069.1 | −0.24103121 | −1.17884225 | 0.010692592 | 1.47E−11 | 0.02138 | 0 |
| 1141. | SLC25A24 | NM_013386.3 | −0.28433847 | 1.103286675 | 0.010767317 | 8.54E−10 | 0.02152 | 0 |
| 1142. | HIPK3 | NM_005734.3 | −0.29819454 | 1.554806445 | 0.006930015 | 1.38E−12 | 0.01445 | 0 |
| 1143. | COL1A1 | NM_000088.3 | −0.29920476 | −1.25734786 | 0.003454429 | 1.18E−11 | 0.00765 | 0 |
| 1144. | ZBP1 | NM_030776.1 | −0.32809759 | 1.186552517 | 0.004054499 | 2.43E−10 | 0.00885 | 0 |
| 1145. | SLC30A1 | NM_021194.2 | −0.34623511 | 1.235769639 | 0.00536185 | 5.91E−10 | 0.01144 | 0 |
| 1146. | HBB | NM_000518.4 | −0.3539808 | 1.196265371 | 0.000443389 | 8.37E−12 | 0.00118 | 0 |
| 1147. | REEP5 | NM_005669.4 | −0.35562399 | −1.13758724 | 0.000710849 | 5.38E−11 | 0.00181 | 0 |
| 1148. | PAFAH1B1 | NM_000430.2 | −0.3583053 | −1.23721336 | 0.000353276 | 3.75E−12 | 0.00095 | 0 |
| 1149. | GPD2 | NM_001083112.1 | −0.36517075 | 1.196546422 | 0.000234585 | 4.67E−12 | 0.00066 | 0 |
| 1150. | ALPK2 | NM_052947.3 | −0.38259025 | −1.07997593 | 0.000124942 | 2.34E−11 | 0.00037 | 0 |
| 1151. | IFNB1 | NM_002176.2 | −0.38935446 | 1.043614495 | 0.002459152 | 1.24E−08 | 0.00563 | 0 |
| 1152. | GPM6B | NM_001001995.1 | −0.3997932 | −1.11868219 | 0.000123343 | 2.68E−11 | 0.00037 | 0 |
| 1153. | C1orf58 | NM_144695.2 | −0.40749213 | 1.053917811 | 0.00094079 | 3.47E−09 | 0.00234 | 0 |
| 1154. | CENPF | NM_016343.3 | −0.412248 | −1.02783029 | 4.57E−05 | 4.38E−11 | 0.00015 | 0 |
| 1155. | RBMS2P | XR_019556.2 | −0.45074102 | 1.313174925 | 0.000333757 | 7.03E−11 | 0.00091 | 0 |
| 1156. | GMPR | NM_006877.2 | −0.45186814 | 1.360040022 | 0.000972186 | 2.83E−10 | 0.00242 | 0 |
| 1157. | SLC8A1 | NM_021097.2 | −0.45957574 | 1.045939008 | 1.24E−05 | 3.33E−11 | 4.00E−05 | 0 |
| 1158. | SCAMPI | NM_004866.4 | −0.47485584 | 1.368524278 | 1.21E−05 | 4.52E−13 | 4.00E−05 | 0 |
| 1159. | LAMC1 | NM_002293.2 | −0.51077655 | −1.06119464 | 1.71E−06 | 1.19E−11 | 1.00E−05 | 0 |
| 1160. | SLC29A1 | NM_001078174.1 | −0.51113688 | −1.03438508 | 1.87E−05 | 4.58E−10 | 7.00E−05 | 0 |
| 1161. | RIOK3 | NM_003831.2 | −0.5229374 | 1.132954811 | 0.000117643 | 2.07E−09 | 0.00035 | 0 |
| 1162. | C3orf38 | NM_173824.2 | −0.54578092 | 1.032769768 | 1.67E−05 | 1.23E−09 | 6.00E−05 | 0 |
| 1163. | FGD4 | NM_139241.1 | −0.55922158 | 1.214139906 | 2.95E−06 | 1.11E−11 | 1.00E−05 | 0 |
| 1164. | FEM1B | NM_015322.3 | −0.57078589 | 1.026412754 | 2.11E−05 | 3.96E−09 | 7.00E−05 | 0 |
| 1165. | APOOL | NM_198450.3 | −0.57573228 | 1.369364434 | 1.32E−05 | 1.65E−11 | 5.00E−05 | 0 |
| 1166. | NAMPT | NM_005746.2 | −0.57670041 | 1.209760681 | 2.76E−06 | 1.87E−11 | 1.00E−05 | 0 |
| 1167. | MDM2 | NM_002392.2 | −0.58797092 | 1.078380354 | 7.84E−06 | 7.64E−10 | 3.00E−05 | 0 |
| 1168. | LITAF | NM_004862.2 | −0.58920447 | 1.119550459 | 1.02E−05 | 5.89E−10 | 4.00E−05 | 0 |
| 1169. | AHI1 | NM_017651.3 | −0.59267719 | −1.05352126 | 9.53E−07 | 9.12E−11 | 0 | 0 |
| 1170. | RCAN2 | NM_005822.2 | −0.59682376 | −1.00464437 | 6.30E−07 | 1.43E−10 | 0 | 0 |
| 1171. | RBMS1 | NM_002897.3 | −0.61592056 | 1.104404158 | 3.87E−07 | 2.61E−11 | 0 | 0 |
| 1172. | PDGFRL | NM_006207.1 | −0.622703 | 1.202642815 | 8.04E−07 | 1.70E−11 | 0 | 0 |
| 1173. | MBTPS2 | NM_015884.1 | −0.65389988 | 1.034768609 | 6.43E−07 | 4.26E−10 | 0 | 0 |
| 1174. | NT5C3 | NM_001002010.1 | −0.6591791 | 1.882081623 | 5.40E−08 | 4.32E−16 | 0 | 0 |
| 1175. | DCBLD1 | NM_173674.1 | −0.66702493 | 1.593519185 | 1.50E−07 | 4.36E−14 | 0 | 0 |
| 1176. | OSMR | NM_003999.1 | −0.74169069 | 1.118425735 | 1.60E−08 | 1.45E−11 | 0 | 0 |
| 1177. | SPPL2A | NM_032802.3 | −0.75238691 | 1.124334269 | 5.24E−09 | 4.95E−12 | 0 | 0 |
| 1178. | IL4I1 | NM_172374.1 | −0.75714583 | 1.768767185 | 3.29E−06 | 3.41E−12 | 1.00E−05 | 0 |
| 1179. | LOC400759 | NR_003133.1 | −0.76220171 | 1.031978608 | 5.83E−05 | 1.17E−06 | 0.00018 | 2.00E−05 |
| 1180. | RBMS2 | NM_002898.2 | −0.77198946 | 1.447873733 | 6.48E−08 | 1.38E−12 | 0 | 0 |
| 1181. | FAM62B | NM_020728.1 | −0.79375901 | −1.38724639 | 3.35E−08 | 2.32E−12 | 0 | 0 |
| 1182. | IL6 | NM_000600.1 | −0.80239057 | 1.19847345 | 7.54E−09 | 7.44E−12 | 0 | 0 |
| 1183. | ZC3HAV1 | NM_024625.3 | −0.82334287 | 1.470989986 | 1.24E−07 | 7.17E−12 | 0 | 0 |
| 1184. | ARSK | NM_198150.1 | −0.84351166 | 1.030295348 | 2.97E−09 | 9.74E−11 | 0 | 0 |
| 1185. | LGALS9 | NM_009587.2 | −0.85488357 | 1.706039237 | 3.76E−08 | 2.34E−13 | 0 | 0 |
| 1186. | C15orf48 | NM_032413.2 | −0.8681125 | 1.653430083 | 2.53E−08 | 3.48E−13 | 0 | 0 |
| 1187. | CXCL10 | NM_001565.2 | −0.87657905 | 3.234893251 | 3.47E−07 | 3.32E−17 | 0 | 0 |
| 1188. | CMPK2 | NM_207315.2 | −0.88262315 | 1.350691419 | 2.60E−08 | 1.90E−11 | 0 | 0 |
| 1189. | UGCG | NM_003358.1 | −0.88905185 | 1.446532694 | 1.21E−09 | 2.09E−13 | 0 | 0 |
| 1190. | GBP5 | NM_052942.2 | −0.90943424 | 1.314021934 | 9.28E−09 | 1.70E−11 | 0 | 0 |
| 1191. | EEA1 | NM_003566.2 | −0.96299928 | 1.034083322 | 3.71E−09 | 1.13E−09 | 0 | 0 |
| 1192. | CLIC4 | NM_013943.1 | −0.99759701 | 1.227854278 | 2.58E−09 | 7.34E−11 | 0 | 0 |
| 1193. | TRIM78P | NR_002777.2 | −0.99905444 | 1.697782233 | 1.45E−08 | 1.49E−12 | 0 | 0 |
| 1194. | LEPR | NM_001003679.1 | −1.00018695 | −0.5494339 | 1.42E−08 | 6.22E−05 | 0 | 0.00055 |
| 1195. | C3 | NM_000064.1 | −1.0004553 | 0.24788445 | 1.36E−08 | 0.033098399 | 0 | 0.10003 |
| 1196. | ANXA1 | NM_000700.1 | −1.00085997 | 0.085372445 | 3.94E−11 | 0.279023811 | 0 | 0.47209 |
| 1197. | STARD13 | NM_178006.1 | −1.00129109 | −0.89633388 | 4.93E−09 | 2.95E−08 | 0 | 0 |
| 1198. | TRIM69 | NM_182985.3 | −1.00157132 | 0.263947206 | 8.46E−10 | 0.009323484 | 0 | 0.0368 |
| 1199. | TAGLN2 | NM_003564.1 | −1.00175678 | 0.054949045 | 6.66E−10 | 0.549856028 | 0 | 0.72103 |
| 1200. | TIGD5 | NM_032862.2 | −1.00280461 | −0.21629119 | 7.66E−10 | 0.027983442 | 0 | 0.08778 |
| 1201. | MZF1 | NM_198055.1 | −1.00402759 | 0.055575913 | 8.86E−09 | 0.604934683 | 0 | 0.76307 |
| 1202. | LOC338758 | XM_931359.2 | −1.00460172 | −0.07165599 | 1.51E−10 | 0.398871092 | 0 | 0.59273 |
| 1203. | HOXA5 | NM_019102.2 | −1.00487033 | −0.17592457 | 2.05E−10 | 0.050905611 | 0 | 0.13942 |
| 1204. | DIS3L | NM_133375.2 | −1.00527399 | −0.27282182 | 1.16E−09 | 0.008704447 | 0 | 0.0349 |
| 1205. | CKAP4 | NM_006825.2 | −1.00553048 | 0.035382271 | 3.69E−11 | 0.649924815 | 0 | 0.79516 |
| 1206. | ATRIP | NM_032166.2 | −1.00586522 | 0.00213758 | 6.12E−11 | 0.978691922 | 0 | 0.99057 |
| 1207. | EBPL | NM_032565.2 | −1.00639331 | −0.09009877 | 2.04E−11 | 0.239661944 | 0 | 0.42565 |
| 1208. | REC8 | NM_005132.2 | −1.00688308 | −0.24397279 | 3.88E−09 | 0.025354659 | 0 | 0.08155 |
| 1209. | PHYH | NM_001037537.1 | −1.00704487 | −0.57574982 | 1.81E−10 | 1.33E−06 | 0 | 2.00E−05 |
| 1210. | TMEM9 | NM_016456.2 | −1.00750993 | −0.20518529 | 9.19E−11 | 0.019955494 | 0 | 0.06746 |
| 1211. | CLSTN1 | NM_001009566.1 | −1.00859488 | −0.2767763 | 6.84E−11 | 0.00246216 | 0 | 0.01224 |
| 1212. | HSCB | NM_172002.3 | −1.00916402 | 0.17345123 | 4.20E−11 | 0.037214021 | 0 | 0.1096 |
| 1213. | RRM2B | NM_015713.3 | −1.00974531 | 0.616053008 | 7.80E−11 | 2.56E−07 | 0 | 1.00E−05 |
| 1214. | ACVRL1 | NM_000020.1 | −1.00990276 | 0.061988973 | 2.37E−10 | 0.478138188 | 0 | 0.66321 |
| 1215. | SDSL | NM_138432.2 | −1.01062924 | −0.09242636 | 5.14E−08 | 0.445803048 | 0 | 0.63538 |
| 1216. | NFKB2 | NM_001077493.1 | −1.01108617 | 0.220201533 | 1.21E−11 | 0.006597758 | 0 | 0.02781 |
| 1217. | ALDH2 | NM_000690.2 | −1.0111075 | 0.205252115 | 5.38E−10 | 0.033930312 | 0 | 0.10194 |
| 1218. | ANTXR1 | NM_053034.1 | −1.01289398 | 0.35946479 | 8.37E−10 | 0.000948907 | 0 | 0.00552 |
| 1219. | IL15 | NM_000585.2 | −1.01323643 | −0.10288728 | 5.30E−11 | 0.20758339 | 0 | 0.38576 |
| 1220. | TTC15 | NM_016030.5 | −1.01495273 | −0.11927462 | 4.30E−11 | 0.14290549 | 0 | 0.29751 |
| 1221. | ILVBL | NM_006844.3 | −1.01507386 | −0.28026084 | 9.39E−11 | 0.002706147 | 0 | 0.01324 |
| 1222. | PPM1M | NM_144641.1 | −1.01514806 | −0.23811269 | 8.15E−11 | 0.008251188 | 0 | 0.03339 |
| 1223. | ACSL1 | NM_001995.2 | −1.01526208 | 0.146999817 | 1.55E−08 | 0.199246167 | 0 | 0.37497 |
| 1224. | C15orf52 | NM_207380.1 | −1.01586158 | −0.29561857 | 4.88E−10 | 0.00375115 | 0 | 0.01746 |
| 1225. | ZNF650 | NM_172070.2 | −1.01591905 | 0.690405961 | 1.42E−11 | 1.06E−08 | 0 | 0 |
| 1226. | ATG7 | NM_006395.1 | −1.01606708 | 0.208256973 | 7.39E−09 | 0.063279327 | 0 | 0.1644 |
| 1227. | TGFBR2 | NM_001024847.1 | −1.01698806 | 0.174313232 | 4.10E−10 | 0.06484134 | 0 | 0.16745 |
| 1228. | VAMP4 | NM_003762.3 | −1.01752757 | 0.473105403 | 2.23E−09 | 0.000113827 | 0 | 0.00092 |
| 1229. | BEXL1 | XM_936467.2 | −1.01784735 | −0.15283526 | 2.44E−11 | 0.05801274 | 0 | 0.15396 |
| 1230. | RUNX2 | NM_001024630.2 | −1.01911377 | 0.055237951 | 5.35E−11 | 0.495051574 | 0 | 0.67713 |
| 1231. | VAMP8 | NM_003761.2 | −1.01962958 | 0.002691325 | 3.35E−08 | 0.98182034 | 0 | 0.9919 |
| 1232. | LOC653583 | XM_928224.2 | −1.01970709 | 0.100451843 | 1.62E−10 | 0.249663459 | 0 | 0.43766 |
| 1233. | TERF2 | NM_005652.2 | −1.02002113 | 0.331398728 | 1.36E−09 | 0.002556405 | 0 | 0.01262 |
| 1234. | PTPRA | NM_080841.2 | −1.0200671 | 0.041132719 | 8.37E−11 | 0.619707912 | 0 | 0.77438 |
| 1235. | TRPV2 | NM_016113.3 | −1.02033054 | 0.31160415 | 5.14E−10 | 0.002648347 | 0 | 0.01298 |
| 1236. | Cxorfl2 | NM_003492.1 | −1.02042797 | 0.034624553 | 2.46E−09 | 0.73130807 | 0 | 0.84976 |
| 1237. | RNF146 | NM_030963.2 | −1.02044138 | 0.146286749 | 6.23E−09 | 0.179678608 | 0 | 0.34878 |
| 1238. | GAMT | NM_000156.4 | −1.02058409 | −0.35290292 | 8.79E−10 | 0.00122572 | 0 | 0.00681 |
| 1239. | GLT8D1 | NM_018446.2 | −1.02073656 | −0.27151244 | 4.33E−09 | 0.015882461 | 0 | 0.05639 |
| 1240. | WIPI1 | NM_017983.4 | −1.02164211 | 0.07989414 | 6.59E−11 | 0.33359598 | 0 | 0.53065 |
| 1241. | NTN4 | NM_021229.3 | −1.02216955 | 0.03723997 | 2.31E−08 | 0.747819665 | 0 | 0.86101 |
| 1242. | ZMYM3 | NM_201599.1 | −1.02219273 | −0.01297442 | 3.31E−09 | 0.899441917 | 0 | 0.94953 |
| 1243. | FAM3A | NM_021806.1 | −1.02234696 | −0.18333266 | 4.83E−10 | 0.056482113 | 0 | 0.15086 |
| 1244. | RBM23 | NM_018107.4 | −1.02240319 | −0.07728809 | 8.01E−11 | 0.354820288 | 0 | 0.55116 |
| 1245. | SMARCD3 | NM_003078.3 | −1.02245569 | −0.3421277 | 3.73E−11 | 0.000300327 | 0 | 0.00209 |
| 1246. | C1orf131 | NM_152379.2 | −1.02288052 | −0.08788694 | 4.23E−10 | 0.3396059 | 0 | 0.5365 |
| 1247. | RNF213 | NM_020914.3 | −1.02352275 | 0.630793255 | 3.71E−10 | 8.21E−07 | 0 | 2.00E−05 |
| 1248. | DNAJB4 | NM_007034.3 | −1.02382303 | 0.413005013 | 4.52E−08 | 0.002525128 | 0 | 0.01248 |
| 1249. | ULBP1 | NM_025218.2 | −1.02464222 | 0.619275117 | 1.00E−08 | 1.52E−05 | 0 | 0.00017 |
| 1250. | IDS | NM_000202.3 | −1.02471053 | 0.665503463 | 1.20E−10 | 1.44E−07 | 0 | 0 |
| 1251. | SLC16A5 | NM_004695.2 | −1.02541031 | −0.03968265 | 3.36E−09 | 0.70079185 | 0 | 0.82953 |
| 1252. | TINF2 | NM_012461.1 | −1.02546681 | 0.386138648 | 3.63E−10 | 0.000339715 | 0 | 0.00232 |
| 1253. | GAS6 | NM_000820.1 | −1.02587109 | −0.31870511 | 4.47E−11 | 0.000683668 | 0 | 0.0042 |
| 1254. | SLC43A3 | NM_017611.2 | −1.02596127 | 0.437566237 | 1.02E−09 | 0.000175557 | 0 | 0.00132 |
| 1255. | FAM156A | NM_014138.3 | −1.02743694 | −0.13647211 | 1.98E−10 | 0.129889659 | 0 | 0.27791 |
| 1256. | ASAP2 | NM_003887.2 | −1.02803205 | −0.34231267 | 2.73E−11 | 0.000263984 | 0 | 0.00188 |
| 1257. | GJD3 | NM_152219.3 | −1.02847929 | 0.864574016 | 1.35E−10 | 2.62E−09 | 0 | 0 |
| 1258. | C2orf28 | NM_080592.2 | −1.02874375 | −0.07858913 | 4.95E−11 | 0.33696391 | 0 | 0.53401 |
| 1259. | EFEMP1 | NM_001039348.1 | −1.02978102 | −0.20772504 | 1.74E−10 | 0.025432771 | 0 | 0.08174 |
| 1260. | GSTO1 | NM_004832.1 | −1.03007833 | 0.314163517 | 4.61E−11 | 0.000823775 | 0 | 0.00491 |
| 1261. | D2HGDH | NM_152783.3 | −1.03061752 | −0.3981992 | 3.73E−09 | 0.000983544 | 0 | 0.00569 |
| 1262. | PTEN | NM_000314.4 | −1.03096663 | −0.30285484 | 2.76E−11 | 0.000890422 | 0 | 0.00524 |
| 1263. | RAB13 | NM_002870.2 | −1.03194193 | 0.349571805 | 5.53E−09 | 0.003547841 | 0 | 0.01664 |
| 1264. | APBA3 | NM_004886.3 | −1.03241529 | 0.001776099 | 1.06E−11 | 0.980961543 | 0 | 0.9915 |
| 1265. | MGST1 | NM_145792.1 | −1.03246386 | 0.227081996 | 6.67E−11 | 0.011534287 | 0 | 0.04357 |
| 1266. | MGLL | NM_007283.5 | −1.0334287 | −0.00330488 | 4.32E−11 | 0.967298433 | 0 | 0.98544 |
| 1267. | MOSC1 | NM_022746.2 | −1.03390536 | −0.10838932 | 6.41E−08 | 0.389841994 | 0 | 0.58474 |
| 1268. | ZNF672 | NM_024836.1 | −1.03400454 | −0.14813865 | 8.74E−10 | 0.133783769 | 0 | 0.28365 |
| 1269. | CDC42EP4 | NM_012121.4 | −1.03422592 | 0.066327892 | 5.16E−11 | 0.419663147 | 0 | 0.61177 |
| 1270. | NUDT18 | NM_024815.3 | −1.03435449 | 0.169208751 | 2.09E−10 | 0.066744183 | 0 | 0.17107 |
| 1271. | MXD4 | NM_006454.2 | −1.03509964 | −0.91679597 | 5.02E−09 | 3.56E−08 | 0 | 0 |
| 1272. | TMEM189-UBE2V1 | NM_199203.1 | −1.03579522 | 0.063781062 | 7.55E−10 | 0.505651759 | 0 | 0.68575 |
| 1273. | SLC9A3R1 | NM_004252.2 | −1.03608282 | −0.06339429 | 5.02E−11 | 0.440271783 | 0 | 0.63064 |
| 1274. | DENND5A | NM_015213.2 | −1.03650108 | 0.079913603 | 8.79E−12 | 0.28696254 | 0 | 0.48133 |
| 1275. | HOXB7 | NM_004502.3 | −1.03685924 | −0.06123515 | 4.39E−11 | 0.45275097 | 0 | 0.64195 |
| 1276. | PACSIN2 | NM_007229.1 | −1.03777942 | −0.32531045 | 3.09E−11 | 0.000515457 | 0 | 0.00331 |
| 1277. | ACCS | NM_032592.3 | −1.03842113 | −0.15573423 | 1.20E−10 | 0.081305814 | 0 | 0.19845 |
| 1278. | XPO1 | NM_003400.3 | −1.03964261 | −0.23248685 | 3.98E−10 | 0.018989932 | 0 | 0.06495 |
| 1279. | C20orf72 | NM_052865.2 | −1.03987237 | −0.05253853 | 5.69E−11 | 0.525881169 | 0 | 0.70174 |
| 1280. | ITGA3 | NM_002204.1 | −1.04102345 | 0.126786496 | 6.97E−11 | 0.139776672 | 0 | 0.29239 |
| 1281. | IRF2BP2 | NM_182972.2 | −1.04105083 | −0.80601308 | 1.76E−10 | 1.30E−08 | 0 | 0 |
| 1282. | SRC | NM_198291.1 | −1.04134599 | 0.056904895 | 3.78E−11 | 0.483095417 | 0 | 0.6673 |
| 1283. | LOC285296 | XM_001714301.1 | −1.04185053 | 0.824626417 | 6.62E−11 | 3.62E−09 | 0 | 0 |
| 1284. | MYF6 | NM_002469.1 | −1.04218609 | −0.02360164 | 3.67E−10 | 0.797483378 | 0 | 0.89132 |
| 1285. | ZFP36 | NM_003407.2 | −1.04372218 | 0.231703522 | 7.05E−11 | 0.011107992 | 0 | 0.04224 |
| 1286. | C4orf34 | NM_174921.1 | −1.04434188 | 0.611877122 | 5.29E−12 | 4.71E−08 | 0 | 0 |
| 1287. | FAM57A | NM_024792.1 | −1.04479032 | −0.00313515 | 9.16E−10 | 0.9742696 | 0 | 0.98794 |
| 1288. | LSS | NM_002340.3 | −1.04498765 | −0.00730124 | 9.00E−11 | 0.931544337 | 0 | 0.96702 |
| 1289. | MME | NM_000902.3 | −1.04587432 | −0.18403688 | 7.15E−11 | 0.038349759 | 0 | 0.11221 |
| 1290. | SULF2 | NM_018837.2 | −1.04795874 | −0.04816615 | 3.65E−11 | 0.553792099 | 0 | 0.72451 |
| 1291. | ATP6AP1L | NM_001017971.1 | −1.04946395 | 0.02802534 | 7.28E−11 | 0.739990972 | 0 | 0.85518 |
| 1292. | RCOR2 | NM_173587.2 | −1.05065782 | −0.19485673 | 1.98E−09 | 0.068327822 | 0 | 0.1741 |
| 1293. | FAM160B1 | NM_020940.3 | −1.05090412 | 0.628169983 | 3.44E−10 | 1.19E−06 | 0 | 2.00E−05 |
| 1294. | RFX5 | NM_001025603.1 | −1.05207078 | −0.06022618 | 1.38E−11 | 0.43746356 | 0 | 0.62822 |
| 1295. | FAM65A | NM_024519.2 | −1.05253845 | −0.12629991 | 1.27E−10 | 0.15875063 | 0 | 0.32029 |
| 1296. | TRIM55 | NM_184086.1 | −1.05288688 | 0.196695804 | 1.84E−11 | 0.019444539 | 0 | 0.06604 |
| 1297. | MYPOP | NM_001012643.2 | −1.05317044 | −0.03552199 | 2.19E−11 | 0.654134469 | 0 | 0.79802 |
| 1298. | TCFL5 | NM_006602.2 | −1.05337852 | 0.033604893 | 3.48E−12 | 0.638799111 | 0 | 0.78755 |
| 1299. | GPR1 | NM_001098199.1 | −1.05391401 | 0.220118451 | 3.83E−11 | 0.01303422 | 0 | 0.04819 |
| 1300. | IGDCC4 | NM_020962.1 | −1.05413923 | −0.52159292 | 2.30E−11 | 1.94E−06 | 0 | 3.00E−05 |
| 1301. | ARHGEF2 | NM_004723.2 | −1.05674634 | −0.07135827 | 6.16E−11 | 0.400540634 | 0 | 0.59451 |
| 1302. | CCND2 | NM_001759.2 | −1.05683056 | −0.52817599 | 1.20E−09 | 3.25E−05 | 0 | 0.00032 |
| 1303. | EEF2K | NM_013302.3 | −1.05774489 | −0.53218489 | 1.24E−11 | 9.44E−07 | 0 | 2.00E−05 |
| 1304. | CASP4 | NM_001225.3 | −1.05930929 | 0.690796965 | 4.20E−11 | 5.41E−08 | 0 | 0 |
| 1305. | RNASE4 | NM_194431.1 | −1.06008853 | −0.1107261 | 2.08E−10 | 0.2296911 | 0 | 0.41321 |
| 1306. | AUH | NM_001698.1 | −1.06019572 | 0.160413368 | 2.35E−11 | 0.05577631 | 0 | 0.14961 |
| 1307. | TDRD7 | NM_014290.1 | −1.06193552 | 1.072280332 | 1.18E−09 | 1.01E−09 | 0 | 0 |
| 1308. | RCN3 | NM_020650.2 | −1.06243068 | −0.20210498 | 2.00E−11 | 0.01808814 | 0 | 0.06246 |
| 1309. | JUNB | NM_002229.2 | −1.06337011 | 0.515245722 | 1.65E−10 | 1.14E−05 | 0 | 0.00013 |
| 1310. | HAS1 | NM_001523.1 | −1.06338584 | 0.217026478 | 5.25E−09 | 0.059345921 | 0 | 0.1566 |
| 1311. | C14orf93 | NM_021944.1 | −1.06340255 | −0.10731526 | 3.28E−11 | 0.198485007 | 0 | 0.37388 |
| 1312. | PPP2R2C | NM_181876.2 | −1.06387129 | −0.23080346 | 6.30E−10 | 0.025665245 | 0 | 0.08234 |
| 1313. | ANGPTL4 | NM_139314.1 | −1.06440002 | 1.266897552 | 1.39E−09 | 6.94E−11 | 0 | 0 |
| 1314. | FXYD5 | NM_144779.1 | −1.06511972 | 0.094610302 | 3.23E−11 | 0.255044755 | 0 | 0.4434 |
| 1315. | HOM-TES-103 | NM_080731.1 | −1.06515635 | −0.31655432 | 1.51E−10 | 0.001860683 | 0 | 0.0097 |
| 1316. | FLT3LG | NM_001459.2 | −1.0654256 | 0.38728166 | 5.98E−11 | 0.000167558 | 0 | 0.00127 |
| 1317. | GBA | NM_001005742.1 | −1.06545864 | 0.288978323 | 2.35E−11 | 0.001652062 | 0 | 0.00878 |
| 1318. | ETS1 | NM_005238.2 | −1.06666678 | −0.1783118 | 8.53E−09 | 0.127470143 | 0 | 0.2745 |
| 1319. | THY1 | NM_006288.2 | −1.06677074 | −0.64440098 | 1.24E−11 | 6.13E−08 | 0 | 0 |
| 1320. | AARS | NM_001605.2 | −1.06728577 | 0.277572737 | 6.67E−11 | 0.003689333 | 0 | 0.01725 |
| 1321. | NDST1 | NM_001543.3 | −1.06902065 | −0.69119706 | 1.51E−09 | 1.34E−06 | 0 | 2.00E−05 |
| 1322. | ALOX15B | NM_001141.2 | −1.07066744 | 0.451755678 | 9.89E−10 | 0.000192752 | 0 | 0.00143 |
| 1323. | TAX1BP3 | NM_014604.2 | −1.07230743 | −0.34095702 | 6.77E−11 | 0.000685354 | 0 | 0.0042 |
| 1324. | RILPL1 | NM_178314.2 | −1.0723764 | 0.623268364 | 5.01E−10 | 2.40E−06 | 0 | 4.00E−05 |
| 1325. | FEZ1 | NM_022549.2 | −1.07316721 | 0.189981814 | 5.49E−11 | 0.03482 | 0 | 0.10397 |
| 1326. | KTELC1 | NM_020231.3 | −1.07325158 | 0.156414048 | 8.86E−12 | 0.052061477 | 0 | 0.14186 |
| 1327. | RHOT1 | NM_001033566.1 | −1.07434479 | 0.330750314 | 3.74E−12 | 0.000188365 | 0 | 0.0014 |
| 1328. | MAP3K6 | NM_004672.3 | −1.07445986 | −0.22682833 | 3.93E−11 | 0.012330138 | 0 | 0.04603 |
| 1329. | TBC1D2 | NM_018421.2 | −1.07454756 | 0.292616438 | 6.85E−10 | 0.006929103 | 0 | 0.02898 |
| 1330. | FAM189B | NM_006589.2 | −1.07523534 | 0.040193947 | 1.48E−10 | 0.655893355 | 0 | 0.79969 |
| 1331. | NUP37 | NM_024057.2 | −1.075314 | −0.13391316 | 1.11E−11 | 0.096579133 | 0 | 0.22414 |
| 1332. | PSAT1 | NM_021154.3 | −1.07567406 | 0.736451787 | 3.35E−11 | 2.05E−08 | 0 | 0 |
| 1333. | PLEKHA2 | NM_021623.1 | −1.07712827 | 0.062003171 | 4.81E−11 | 0.466371544 | 0 | 0.65369 |
| 1334. | BID | NM_197966.1 | −1.07811035 | −0.16745726 | 8.96E−12 | 0.039744067 | 0 | 0.11553 |
| 1335. | HS1BP3 | NM_022460.3 | −1.07839537 | 0.051425836 | 2.84E−11 | 0.533427888 | 0 | 0.70789 |
| 1336. | ARSD | NM_001669.2 | −1.07860998 | −0.20520685 | 1.08E−10 | 0.029680584 | 0 | 0.09185 |
| 1337. | GSTK1 | NM_015917.1 | −1.07866338 | 0.059298016 | 4.51E−12 | 0.427729021 | 0 | 0.61948 |
| 1338. | ACPL2 | NM_152282.3 | −1.07921904 | 0.14679038 | 6.29E−11 | 0.099555678 | 0 | 0.22935 |
| 1339. | RNF31 | NM_017999.4 | −1.07984343 | 0.051415213 | 1.87E−10 | 0.576016056 | 0 | 0.74068 |
| 1340. | TBK1 | NM_013254.2 | −1.08002803 | 0.357183055 | 3.94E−11 | 0.000348733 | 0 | 0.00237 |
| 1341. | NOTCH1 | NM_017617.3 | −1.08006 | −0.66953259 | 2.93E−09 | 4.13E−06 | 0 | 6.00E−05 |
| 1342. | C20orf117 | NM_199181.2 | −1.08011362 | −0.53217661 | 2.09E−11 | 1.91E−06 | 0 | 3.00E−05 |
| 1343. | HLA-A | NM_002116.5 | −1.08013352 | 0.557697842 | 1.59E−08 | 0.000134164 | 0 | 0.00105 |
| 1344. | CYFIP2 | NM_001037333.1 | −1.08099479 | −0.63302219 | 2.29E−11 | 1.67E−07 | 0 | 0 |
| 1345. | ATP2B1 | NM_001001323.1 | −1.0817218 | −0.70390831 | 4.82E−09 | 3.21E−06 | 0 | 5.00E−05 |
| 1346. | ZKSCAN1 | NM_003439.1 | −1.0817446 | 0.380240593 | 1.65E−08 | 0.004447165 | 0 | 0.0201 |
| 1347. | C8orf55 | NM_016647.2 | −1.0823364 | −0.38704721 | 3.80E−11 | 0.000151713 | 0 | 0.00117 |
| 1348. | CLIC1 | NM_001288.4 | −1.08331855 | 0.308893162 | 6.15E−12 | 0.000538018 | 0 | 0.00343 |
| 1349. | ARHGAP22 | NM_021226.2 | −1.08424685 | 0.077731251 | 2.72E−11 | 0.351028366 | 0 | 0.54746 |
| 1350. | NADSYN1 | NM_018161.4 | −1.08625609 | 0.118208324 | 2.30E−11 | 0.158998903 | 0 | 0.32067 |
| 1351. | DRAM1 | NM_018370.2 | −1.08687246 | −0.36488141 | 4.65E−11 | 0.000329433 | 0 | 0.00226 |
| 1352. | SHMT2 | NM_005412.4 | −1.08862162 | 0.261511532 | 1.09E−10 | 0.00787209 | 0 | 0.03216 |
| 1353. | PTGFR | NM_000959.3 | −1.08884041 | 0.149623299 | 2.17E−10 | 0.119685358 | 0 | 0.26244 |
| 1354. | CXCL16 | NM_022059.1 | −1.09023442 | 1.183509176 | 9.24E−11 | 2.17E−11 | 0 | 0 |
| 1355. | CD68 | NM_001251.1 | −1.0912592 | 1.175059517 | 3.43E−10 | 9.47E−11 | 0 | 0 |
| 1356. | NRSN2 | NM_024958.1 | −1.09142027 | 0.068072359 | 2.96E−12 | 0.358447094 | 0 | 0.55456 |
| 1357. | LOC730278 | XM_001126471.1 | −1.09182645 | 0.189911348 | 7.13E−10 | 0.069266329 | 0 | 0.176 |
| 1358. | THBS1 | NM_003246.2 | −1.09190365 | −0.56593524 | 4.24E−11 | 1.64E−06 | 0 | 3.00E−05 |
| 1359. | NFE2L2 | NM_006164.2 | −1.09267987 | 0.32900533 | 9.58E−12 | 0.000402512 | 0 | 0.00268 |
| 1360. | LOC728060 | XR_015272.2 | −1.09472957 | 0.25532926 | 8.89E−11 | 0.008837118 | 0 | 0.03525 |
| 1361. | AP1S1 | NM_057089.2 | −1.09532783 | −0.14035425 | 8.83E−10 | 0.177712026 | 0 | 0.3462 |
| 1362. | CAV1 | NM_001753.3 | −1.09664018 | −0.19112993 | 4.09E−11 | 0.03465702 | 0 | 0.10359 |
| 1363. | BEX1 | NM_018476.3 | −1.09691667 | −0.23461683 | 4.30E−11 | 0.011748596 | 0 | 0.04419 |
| 1364. | PRKD1 | NM_002742.2 | −1.09863799 | −0.6085326 | 4.20E−11 | 6.33E−07 | 0 | 1.00E−05 |
| 1365. | WEE1 | NM_003390.2 | −1.09868601 | 0.332519669 | 3.81E−10 | 0.002478081 | 0 | 0.0123 |
| 1366. | PRKAG2 | NM_024429.1 | −1.09879065 | 0.754723456 | 6.53E−12 | 4.39E−09 | 0 | 0 |
| 1367. | HCP5 | NM_006674.2 | −1.09901267 | 0.144295449 | 1.82E−11 | 0.088959444 | 0 | 0.21176 |
| 1368. | SLC24A6 | NM_024959.2 | −1.09932642 | 0.00010176 | 8.15E−10 | 0.999200656 | 0 | 0.9996 |
| 1369. | C2orf32 | NM_015463.1 | −1.09974886 | 0.078131125 | 1.18E−10 | 0.394204315 | 0 | 0.58867 |
| 1370. | DPYSL2 | NM_001386.4 | −1.09989485 | 0.228537719 | 1.59E−11 | 0.009925877 | 0 | 0.03863 |
| 1371. | ETV6 | NM_001987.4 | −1.1000486 | −0.0553284 | 1.19E−10 | 0.544679472 | 0 | 0.71702 |
| 1372. | ODF3B | NM_001014440.2 | −1.10039204 | 0.42956235 | 1.46E−11 | 3.03E−05 | 0 | 3.00E−04 |
| 1373. | PEX16 | NM_004813.1 | −1.10070279 | 0.024268324 | 5.42E−10 | 0.8070527 | 0 | 0.89681 |
| 1374. | SH3PXD2B | NM_001017995.1 | −1.10111105 | −0.01555465 | 9.36E−12 | 0.843659438 | 0 | 0.91755 |
| 1375. | BEST1 | NM_004183.2 | −1.1014659 | 0.266093011 | 1.05E−11 | 0.00292983 | 0 | 0.01419 |
| 1376. | LRP10 | NM_014045.3 | −1.10241025 | 0.415789662 | 7.02E−12 | 2.80E−05 | 0 | 0.00028 |
| 1377. | PMP22 | NM_153321.1 | −1.10263074 | 0.266687734 | 1.39E−10 | 0.008249178 | 0 | 0.03339 |
| 1378. | RIPK1 | NM_003804.3 | −1.10270158 | −0.24065996 | 1.05E−09 | 0.028965073 | 0 | 0.09019 |
| 1379. | WNT5A | NM_003392.3 | −1.10285871 | −0.39934446 | 4.58E−10 | 0.000568039 | 0 | 0.00359 |
| 1380. | LOC390530 | XM_372543.2 | −1.10354111 | 0.10829986 | 1.40E−09 | 0.310194598 | 0 | 0.5057 |
| 1381. | TXNRD2 | NM_006440.3 | −1.10446667 | −0.16661747 | 1.31E−10 | 0.081135275 | 0 | 0.19814 |
| 1382. | ENG | NM_000118.1 | −1.1045979 | 0.492655113 | 2.40E−10 | 4.11E−05 | 0 | 0.00039 |
| 1383. | MOBKL2C | NM_145279.4 | −1.10570526 | 0.127380024 | 1.17E−09 | 0.231058038 | 0 | 0.41488 |
| 1384. | PPAP2A | NM_176895.1 | −1.10579812 | 0.544950169 | 3.75E−12 | 4.86E−07 | 0 | 1.00E−05 |
| 1385. | PFKP | NM_002627.3 | −1.10605138 | 0.775927102 | 2.83E−10 | 9.46E−08 | 0 | 0 |
| 1386. | HOXC13 | NM_017410.2 | −1.1072319 | 0.443969061 | 7.06E−11 | 6.28E−05 | 0 | 0.00056 |
| 1387. | IL10RB | NM_000628.3 | −1.1077727 | −0.05056423 | 5.67E−12 | 0.514424403 | 0 | 0.69225 |
| 1388. | APOBEC3C | NM_014508.2 | −1.10809902 | 0.58273362 | 2.15E−10 | 4.77E−06 | 0 | 7.00E−05 |
| 1389. | FES | NM_002005.2 | −1.1084545 | −0.33138799 | 2.07E−09 | 0.00572525 | 0 | 0.02471 |
| 1390. | P4HB | NM_000918.3 | −1.10933616 | −0.19271039 | 3.57E−11 | 0.033945125 | 0 | 0.10198 |
| 1391. | HLA-E | NM_005516.4 | −1.11004281 | 0.814748655 | 4.85E−11 | 9.34E−09 | 0 | 0 |
| 1392. | BCL9L | NM_182557.2 | −1.11143745 | 0.732633231 | 3.58E−09 | 2.11E−06 | 0 | 3.00E−05 |
| 1393. | ZBTB25 | NM_006977.2 | −1.11264019 | 0.346301575 | 6.27E−12 | 0.000226809 | 0 | 0.00165 |
| 1394. | FAM156B | NM_001099684.1 | −1.11275453 | −0.17123139 | 2.46E−11 | 0.052634938 | 0 | 0.14315 |
| 1395. | SPG7 | NM_003119.2 | −1.11281034 | −0.27955345 | 1.99E−10 | 0.007357358 | 0 | 0.03043 |
| 1396. | MYOD1 | NM_002478.4 | −1.11297689 | −0.46196618 | 5.42E−11 | 3.51E−05 | 0 | 0.00034 |
| 1397. | NPEPL1 | NM_024663.3 | −1.11311504 | −0.2630971 | 1.91E−10 | 0.010720106 | 0 | 0.041 |
| 1398. | OPTN | NM_001008213.1 | −1.1134359 | −0.572683 | 4.48E−12 | 2.99E−07 | 0 | 1.00E−05 |
| 1399. | TRIM5 | NM_033034.1 | −1.11392597 | 0.500305535 | 7.09E−11 | 1.61E−05 | 0 | 0.00018 |
| 1400. | GRAMD3 | NM_023927.1 | −1.11531797 | 0.377216655 | 8.33E−11 | 0.000425196 | 0 | 0.00281 |
| 1401. | RFNG | XM_001132711.1 | −1.11554495 | −0.21827894 | 8.64E−12 | 0.011678522 | 0 | 0.04397 |
| 1402. | PDE7B | NM_018945.3 | −1.11590806 | −0.56949559 | 2.06E−10 | 6.86E−06 | 0 | 9.00E−05 |
| 1403. | ARMCX1 | NM_016608.1 | −1.11598479 | −0.06031382 | 1.88E−10 | 0.526265431 | 0 | 0.70214 |
| 1404. | FRMD3 | NM_174938.3 | −1.11602118 | −0.29070138 | 1.81E−12 | 0.000667962 | 0 | 0.00412 |
| 1405. | C1orf57 | NM_032324.1 | −1.11721778 | 0.354382035 | 2.69E−11 | 0.000425597 | 0 | 0.00281 |
| 1406. | PRMT2 | NM_001535.2 | −1.11885544 | 0.074119142 | 5.94E−11 | 0.408451827 | 0 | 0.60212 |
| 1407. | ZNF319 | NM_020807.1 | −1.11891714 | −0.08178541 | 8.56E−12 | 0.31132616 | 0 | 0.50689 |
| 1408. | SLC2A10 | NM_030777.3 | −1.11896209 | −0.31968771 | 2.99E−11 | 0.00118681 | 0 | 0.00663 |
| 1409. | CDR2L | NM_014603.1 | −1.11911242 | −0.20301163 | 1.21E−12 | 0.009511858 | 0 | 0.03735 |
| 1410. | CORO6 | NM_032854.2 | −1.12027819 | −0.3003946 | 6.27E−10 | 0.007421803 | 0 | 0.03065 |
| 1411. | LBA1 | NM_014831.1 | −1.12086965 | 0.495819259 | 5.43E−10 | 7.83E−05 | 0 | 0.00067 |
| 1412. | CERCAM | NM_016174.3 | −1.12092245 | −0.18379352 | 8.87E−11 | 0.054639249 | 0 | 0.14726 |
| 1413. | CTDSP1 | NM_021198.1 | −1.12174762 | 0.041546061 | 3.63E−11 | 0.632601165 | 0 | 0.78317 |
| 1414. | PCBP3 | NM_020528.1 | −1.12178489 | 0.211347399 | 3.76E−10 | 0.04325855 | 0 | 0.12317 |
| 1415. | TIAF1 | NM_004740.3 | −1.12263209 | −0.52935476 | 1.55E−11 | 2.78E−06 | 0 | 4.00E−05 |
| 1416. | LPXN | NM_004811.1 | −1.12383405 | −0.01760094 | 2.14E−11 | 0.834629425 | 0 | 0.91265 |
| 1417. | TYMS | NM_001071.1 | −1.1252785 | −0.09199154 | 7.27E−12 | 0.254866653 | 0 | 0.4433 |
| 1418. | FKBP11 | NM_016594.1 | −1.12528495 | 0.300590026 | 1.63E−11 | 0.001581497 | 0 | 0.00846 |
| 1419. | PSRC1 | NM_001032290.1 | −1.12569454 | −0.48705968 | 1.13E−10 | 3.52E−05 | 0 | 0.00034 |
| 1420. | FAM129B | NM_022833.2 | −1.12586668 | 0.438215519 | 1.32E−11 | 2.93E−05 | 0 | 3.00E−04 |
| 1421. | LUM | NM_002345.3 | −1.12592585 | 0.386922869 | 5.27E−11 | 0.000279368 | 0 | 0.00197 |
| 1422. | LYRM1 | NM_020424.2 | −1.12605499 | 0.014914193 | 8.10E−13 | 0.832436921 | 0 | 0.91152 |
| 1423. | TNFRSF10A | NM_003844.2 | −1.12617958 | 0.844102309 | 1.25E−10 | 1.59E−08 | 0 | 0 |
| 1424. | ACP2 | NM_001610.1 | −1.12624234 | 0.222500267 | 2.14E−10 | 0.02992589 | 0 | 0.09239 |
| 1425. | SYNC1 | NM_030786.1 | −1.12665499 | −0.25080558 | 3.63E−09 | 0.037412442 | 0 | 0.11009 |
| 1426. | USP41 | XM_036729.5 | −1.12677811 | 0.524575184 | 2.23E−11 | 4.34E−06 | 0 | 6.00E−05 |
| 1427. | TNFSF10 | NM_003810.2 | −1.12685752 | 2.971672323 | 3.25E−08 | 1.06E−15 | 0 | 0 |
| 1428. | LOC645638 | XR_040455.1 | −1.12764118 | −0.07112827 | 1.14E−11 | 0.388206869 | 0 | 0.58348 |
| 1429. | PID1 | NM_017933.3 | −1.12788845 | 0.314029719 | 1.09E−11 | 0.000903744 | 0 | 0.00531 |
| 1430. | TOM1 | NM_005488.1 | −1.12875228 | 4.81E−05 | 6.50E−12 | 0.999515526 | 0 | 0.99971 |
| 1431. | GBE1 | NM_000158.2 | −1.12885253 | 0.481765414 | 3.55E−12 | 3.53E−06 | 0 | 5.00E−05 |
| 1432. | PNPO | NM_018129.2 | −1.13041513 | −0.02587896 | 1.55E−11 | 0.756193528 | 0 | 0.86627 |
| 1433. | LHFPL2 | NM_005779.1 | −1.13042882 | −0.14372411 | 3.01E−09 | 0.212228234 | 0 | 0.39165 |
| 1434. | CST3 | NM_000099.2 | −1.13121662 | 0.157381243 | 8.62E−12 | 0.062234167 | 0 | 0.16234 |
| 1435. | SLC44A1 | NM_080546.3 | −1.13216786 | 0.780099421 | 7.54E−12 | 4.76E−09 | 0 | 0 |
| 1436. | SLC35E3 | NM_018656.2 | −1.13240472 | 0.461873605 | 8.16E−11 | 5.67E−05 | 0 | 0.00051 |
| 1437. | RXRA | NM_002957.3 | −1.13242382 | −0.32017726 | 3.82E−12 | 0.00045236 | 0 | 0.00296 |
| 1438. | CREB1 | NM_004379.2 | −1.13307916 | −0.08534387 | 2.95E−12 | 0.269699958 | 0 | 0.46109 |
| 1439. | FAS | NM_152877.1 | −1.13338054 | 0.145223093 | 6.87E−11 | 0.121108539 | 0 | 0.26477 |
| 1440. | SLC22A4 | NM_003059.2 | −1.13425744 | 0.381696472 | 2.78E−10 | 0.000858012 | 0 | 0.00508 |
| 1441. | NOD2 | NM_022162.1 | −1.13496736 | 0.434713135 | 2.09E−11 | 4.81E−05 | 0 | 0.00045 |
| 1442. | ASB5 | NM_080874.2 | −1.13535655 | −0.4567075 | 2.29E−11 | 2.85E−05 | 0 | 0.00029 |
| 1443. | IFI27L2 | NM_032036.2 | −1.13681021 | −0.02949474 | 7.49E−10 | 0.778016481 | 0 | 0.87915 |
| 1444. | CDC42EP5 | NM_145057.2 | −1.13701196 | 0.138705526 | 6.69E−10 | 0.191469223 | 0 | 0.36534 |
| 1445. | TP53INP1 | NM_033285.2 | −1.13775234 | −0.57336685 | 1.81E−10 | 7.33E−06 | 0 | 9.00E−05 |
| 1446. | PSME1 | NM_006263.2 | −1.13780064 | 0.277504124 | 1.77E−12 | 0.001200584 | 0 | 0.00669 |
| 1447. | FAT1 | NM_005245.3 | −1.13818248 | 0.40548035 | 1.04E−10 | 0.000286767 | 0 | 0.00201 |
| 1448. | CRTC3 | NM_022769.3 | −1.13844672 | −0.1184239 | 1.30E−11 | 0.164245027 | 0 | 0.32791 |
| 1449. | LOC650215 | XR_018889.1 | −1.13895881 | 0.63860381 | 1.45E−12 | 3.07E−08 | 0 | 0 |
| 1450. | EMP3 | NM_001425.1 | −1.13943282 | −0.2096233 | 4.38E−11 | 0.027188079 | 0 | 0.0858 |
| 1451. | TRIM56 | NM_030961.1 | −1.13956107 | 0.308557613 | 2.89E−11 | 0.001845441 | 0 | 0.00964 |
| 1452. | LOC728809 | XM_001719546.1 | −1.14043153 | 0.022132969 | 8.26E−11 | 0.810618999 | 0 | 0.89891 |
| 1453. | C11orf68 | NM_031450.2 | −1.14152841 | 0.36162395 | 1.71E−11 | 0.000335143 | 0 | 0.0023 |
| 1454. | PEX11B | NM_003846.1 | −1.14215923 | −0.22122472 | 7.10E−11 | 0.024066538 | 0 | 0.07834 |
| 1455. | PDE4B | NM_002600.3 | −1.14298689 | 0.675737387 | 1.50E−11 | 1.01E−07 | 0 | 0 |
| 1456. | LOC653506 | XM_927769.1 | −1.1440126 | 0.017475514 | 1.03E−11 | 0.832048635 | 0 | 0.91125 |
| 1457. | CBLN3 | NM_001039771.2 | −1.14406425 | 0.154256286 | 7.01E−11 | 0.104314005 | 0 | 0.23731 |
| 1458. | OSBPL7 | NM_145798.2 | −1.14474535 | −0.36688245 | 2.21E−11 | 0.000344523 | 0 | 0.00235 |
| 1459. | HIF1A | NM_181054.1 | −1.14976405 | 0.420889531 | 1.73E−07 | 0.009415561 | 0 | 0.0371 |
| 1460. | MFGE8 | NM_005928.1 | −1.14980935 | 0.032536086 | 1.42E−11 | 0.699932682 | 0 | 0.829 |
| 1461. | LASP1 | NM_006148.1 | −1.15156327 | −0.45518784 | 5.84E−11 | 6.54E−05 | 0 | 0.00058 |
| 1462. | ATP6V0A1 | NM_005177.3 | −1.15218372 | −0.04496838 | 1.54E−11 | 0.597306092 | 0 | 0.75747 |
| 1463. | KCNK2 | NM_001017425.2 | −1.15404288 | 0.213670594 | 5.89E−12 | 0.014302853 | 0 | 0.05175 |
| 1464. | ZC3H5 | XM_940903.2 | −1.15463553 | 0.096519659 | 1.10E−10 | 0.31594441 | 0 | 0.51185 |
| 1465. | LTBR | NM_002342.1 | −1.15479546 | 0.406067224 | 3.19E−11 | 0.000163502 | 0 | 0.00125 |
| 1466. | TXLNA | NM_175852.3 | −1.15663244 | −0.16212822 | 3.89E−11 | 0.082358746 | 0 | 0.20036 |
| 1467. | SMARCAL1 | NM_014140.2 | −1.15668354 | 0.016691468 | 5.28E−11 | 0.854947241 | 0 | 0.92393 |
| 1468. | TOP2A | NM_001067.2 | −1.15703548 | −0.60645497 | 5.01E−11 | 1.60E−06 | 0 | 3.00E−05 |
| 1469. | IRS1 | NM_005544.1 | −1.15737634 | 0.275274975 | 3.14E−12 | 0.001939901 | 0 | 0.01004 |
| 1470. | SLC44A2 | NM_020428.2 | −1.15807453 | −0.34567202 | 1.15E−11 | 0.000484719 | 0 | 0.00315 |
| 1471. | CYP4V2 | NM_207352.2 | −1.15927998 | −0.49894739 | 2.01E−11 | 1.12E−05 | 0 | 0.00013 |
| 1472. | MTSS1 | NM_014751.4 | −1.15938641 | −0.4144808 | 7.77E−10 | 0.00085324 | 0 | 0.00506 |
| 1473. | LOC100129165 | XM_001718314.1 | −1.160536 | 0.22453105 | 6.53E−12 | 0.011377742 | 0 | 0.04309 |
| 1474. | PARD6G | NM_032510.3 | −1.16124563 | −0.01048755 | 1.51E−09 | 0.924905714 | 0 | 0.96364 |
| 1475. | KDELR3 | NM_006855.2 | −1.16287967 | −0.44400682 | 1.25E−11 | 3.55E−05 | 0 | 0.00035 |
| 1476. | ADAMTS1 | NM_006988.3 | −1.16353852 | 0.176486006 | 4.09E−10 | 0.099095848 | 0 | 0.22859 |
| 1477. | ZNF302 | NM_018443.2 | −1.16400043 | −0.01005428 | 9.96E−11 | 0.915918023 | 0 | 0.95951 |
| 1478. | PSPH | NM_004577.3 | −1.16402902 | 0.005653554 | 1.18E−11 | 0.946619231 | 0 | 0.97406 |
| 1479. | FRMD4A | NM_018027.3 | −1.16430189 | −0.54533485 | 2.39E−12 | 7.09E−07 | 0 | 1.00E−05 |
| 1480. | LOC644739 | XM_933679.1 | −1.16529261 | −0.07398917 | 1.32E−11 | 0.388790686 | 0 | 0.58399 |
| 1481. | ZMIZ1 | NM_020338.2 | −1.16533295 | −0.03786985 | 1.58E−11 | 0.660091727 | 0 | 0.80268 |
| 1482. | NPR2 | NM_003995.3 | −1.16612668 | −0.04505504 | 7.81E−11 | 0.633005094 | 0 | 0.78323 |
| 1483. | ID1 | NM_181353.1 | −1.16641588 | 0.732925025 | 2.20E−10 | 3.99E−07 | 0 | 1.00E−05 |
| 1484. | NLRX1 | NM_170722.1 | −1.16689384 | −0.14091168 | 1.48E−11 | 0.111746507 | 0 | 0.24973 |
| 1485. | C14orf173 | NM_022489.2 | −1.16706082 | −0.27135286 | 1.26E−12 | 0.001543132 | 0 | 0.00828 |
| 1486. | HECW2 | NM_020760.1 | −1.16735475 | 0.06016212 | 4.75E−12 | 0.458021473 | 0 | 0.64645 |
| 1487. | RAB40C | NM_021168.2 | −1.16811719 | −0.07137997 | 5.03E−10 | 0.498862564 | 0 | 0.68032 |
| 1488. | UGP2 | NM_006759.3 | −1.16853639 | 0.1108138 | 2.32E−11 | 0.217100893 | 0 | 0.39777 |
| 1489. | CASP1 | NM_033294.2 | −1.1695189 | 2.170829048 | 9.36E−11 | 1.09E−15 | 0 | 0 |
| 1490. | CPSF4 | NM_001081559.1 | −1.17121049 | −0.26735466 | 2.30E−12 | 0.002357768 | 0 | 0.01182 |
| 1491. | HIPK2 | NM_022740.2 | −1.1726493 | 0.355894491 | 1.36E−11 | 0.000451829 | 0 | 0.00296 |
| 1492. | MTHFD1L | NM_015440.3 | −1.17347948 | 0.395510823 | 2.09E−09 | 0.002336701 | 0 | 0.01173 |
| 1493. | ABLIM3 | NM_014945.2 | −1.17381801 | 0.014951687 | 6.43E−11 | 0.873205983 | 0 | 0.93426 |
| 1494. | CDH11 | NM_001797.2 | −1.17432107 | 0.241948208 | 7.01E−11 | 0.017195516 | 0 | 0.05996 |
| 1495. | TSPAN4 | NM_001025235.1 | −1.17498403 | −0.1168688 | 1.70E−11 | 0.188981063 | 0 | 0.36212 |
| 1496. | JSRP1 | NM_144616.2 | −1.17536966 | −0.46610287 | 4.52E−11 | 5.33E−05 | 0 | 0.00049 |
| 1497. | FBXO32 | NM_058229.2 | −1.17650838 | 0.766554709 | 7.49E−11 | 9.12E−08 | 0 | 0 |
| 1498. | NCOR2 | NM_001077261.1 | −1.17671645 | −0.22413702 | 1.11E−10 | 0.029713388 | 0 | 0.09193 |
| 1499. | MVP | NM_005115.3 | −1.17773371 | −0.11621563 | 2.34E−10 | 0.258710293 | 0 | 0.44815 |
| 1500. | RAB7L1 | NM_003929.1 | −1.17839739 | 0.431475634 | 3.43E−11 | 0.000110172 | 0 | 0.00089 |
| 1501. | PLOD1 | NM_000302.2 | −1.178952 | −0.14685537 | 9.83E−12 | 0.094426723 | 0 | 0.22099 |
| 1502. | PDE4C | NM_000923.2 | −1.1793672 | 0.39519008 | 2.51E−11 | 0.000235677 | 0 | 0.00171 |
| 1503. | HEG1 | NM_020733.1 | −1.17979515 | −0.25402563 | 6.03E−12 | 0.005461985 | 0 | 0.02376 |
| 1504. | MT1F | NM_005949.2 | −1.18264076 | −0.54040845 | 9.47E−12 | 2.92E−06 | 0 | 4.00E−05 |
| 1505. | CREB3L2 | NM_194071.2 | −1.1832544 | −0.24768779 | 9.38E−12 | 0.007830541 | 0 | 0.03203 |
| 1506. | CAPN5 | NM_004055.4 | −1.18408225 | −0.54314396 | 4.66E−10 | 4.65E−05 | 0 | 0.00043 |
| 1507. | ABCB6 | NM_005689.1 | −1.18409135 | −0.26892257 | 9.07E−12 | 0.004383415 | 0 | 0.01986 |
| 1508. | SYT7 | NM_004200.2 | −1.18426444 | −0.64787893 | 7.44E−10 | 7.35E−06 | 0 | 9.00E−05 |
| 1509. | PTGER2 | NM_000956.2 | −1.18447658 | −0.20234692 | 6.06E−12 | 0.022534326 | 0 | 0.07414 |
| 1510. | BCAR3 | NM_003567.2 | −1.18488673 | −0.14847184 | 9.33E−12 | 0.091752592 | 0 | 0.21651 |
| 1511. | SMYD4 | NM_052928.1 | −1.18510331 | 0.20769913 | 1.41E−11 | 0.025163935 | 0 | 0.08104 |
| 1512. | TRIM8 | NM_030912.2 | −1.18538697 | −0.35362965 | 8.85E−12 | 0.000422075 | 0 | 0.0028 |
| 1513. | ZMYM6 | NM_007167.2 | −1.18603894 | −0.0076299 | 1.93E−12 | 0.921888653 | 0 | 0.96232 |
| 1514. | PLEKHF1 | NM_024310.4 | −1.18680825 | 0.197467933 | 9.92E−10 | 0.086780708 | 0 | 0.20818 |
| 1515. | TMED10P | NR_002807.1 | −1.18683315 | 0.445570742 | 9.38E−12 | 3.59E−05 | 0 | 0.00035 |
| 1516. | FAM174B | NM_207446.2 | −1.18772948 | −0.57106759 | 2.04E−11 | 2.62E−06 | 0 | 4.00E−05 |
| 1517. | C1S | NM_201442.1 | −1.18845168 | 0.420876179 | 2.47E−11 | 0.000129486 | 0 | 0.00102 |
| 1518. | LOC728855 | NR_024510.1 | −1.18875133 | 0.424067129 | 8.83E−11 | 0.000257397 | 0 | 0.00184 |
| 1519. | NEXN | NM_144573.3 | −1.18929137 | −0.26453691 | 3.65E−11 | 0.008666296 | 0 | 0.03478 |
| 1520. | STC2 | NM_003714.2 | −1.18948843 | −0.36095397 | 4.80E−11 | 0.000880696 | 0 | 0.00519 |
| 1521. | AMZ2 | NM_001033569.1 | −1.19097704 | 0.016471304 | 7.00E−12 | 0.844432069 | 0 | 0.91796 |
| 1522. | CAV2 | NM_001233.3 | −1.19188498 | 0.426664771 | 1.65E−12 | 1.99E−05 | 0 | 0.00021 |
| 1523. | SLC9A1 | NM_003047.2 | −1.19305498 | 0.199296174 | 1.81E−11 | 0.034100478 | 0 | 0.10232 |
| 1524. | FYN | NM_153047.1 | −1.19385877 | 0.309464943 | 2.32E−10 | 0.006309822 | 0 | 0.02678 |
| 1525. | POLR3GL | NM_032305.1 | −1.19407945 | −0.09916688 | 2.93E−12 | 0.224900502 | 0 | 0.40707 |
| 1526. | LOC374395 | NM_199337.1 | −1.19589143 | 0.359938265 | 5.26E−13 | 7.44E−05 | 0 | 0.00065 |
| 1527. | IKBKE | NM_014002.2 | −1.19669054 | 0.392291897 | 1.19E−10 | 0.000695024 | 0 | 0.00425 |
| 1528. | TMEM62 | NM_024956.3 | −1.19734084 | 1.21981219 | 6.64E−11 | 4.79E−11 | 0 | 0 |
| 1529. | C1orf66 | NM_015997.2 | −1.19808133 | −0.08346608 | 5.82E−12 | 0.323871883 | 0 | 0.52047 |
| 1530. | SPG21 | NM_016630.3 | −1.19894761 | −0.00716397 | 6.00E−12 | 0.931828283 | 0 | 0.96715 |
| 1531. | C7orf10 | NM_024728.1 | −1.20046313 | −0.317807 | 7.86E−13 | 0.000368054 | 0 | 0.00249 |
| 1532. | CFD | NM_001928.2 | −1.20124234 | 0.252196355 | 1.59E−11 | 0.009307691 | 0 | 0.03675 |
| 1533. | SAMD4A | NM_015589.3 | −1.20155458 | 1.120292909 | 4.96E−10 | 1.63E−09 | 0 | 0 |
| 1534. | APOBEC3F | NM_001006666.1 | −1.2031046 | 0.59819306 | 7.76E−11 | 4.62E−06 | 0 | 6.00E−05 |
| 1535. | SIDT2 | NM_001040455.1 | −1.20512141 | −0.30213165 | 7.56E−13 | 0.000611251 | 0 | 0.00382 |
| 1536. | PHF21A | NM_016621.2 | −1.20733303 | 0.058049641 | 9.48E−13 | 0.449476415 | 0 | 0.63891 |
| 1537. | RPS6KA2 | NM_001006932.1 | −1.20796669 | −0.37413204 | 9.28E−13 | 7.63E−05 | 0 | 0.00066 |
| 1538. | RRAS2 | NM_012250.3 | −1.21247895 | 0.410022176 | 3.59E−12 | 6.54E−05 | 0 | 0.00058 |
| 1539. | ABCA1 | NM_005502.2 | −1.21273165 | −0.32058912 | 2.86E−11 | 0.002235251 | 0 | 0.01131 |
| 1540. | ASPSCR1 | NM_024083.2 | −1.21390751 | −0.30717198 | 4.40E−12 | 0.001346061 | 0 | 0.00737 |
| 1541. | SLC39A8 | NM_022154.5 | −1.21704059 | 0.899188177 | 1.04E−08 | 1.05E−06 | 0 | 2.00E−05 |
| 1542. | BLOC1S2 | NM_001001342.1 | −1.21927547 | 0.446363395 | 1.59E−11 | 6.80E−05 | 0 | 6.00E−04 |
| 1543. | C7orf68 | NM_013332.3 | −1.22027849 | 0.31970722 | 1.22E−11 | 0.001623931 | 0 | 0.00866 |
| 1544. | IFNGR2 | NM_005534.2 | −1.22112757 | 0.022584497 | 1.33E−12 | 0.773769074 | 0 | 0.87642 |
| 1545. | SAA2 | NM_030754.2 | −1.22134171 | 0.281324169 | 6.74E−09 | 0.037996565 | 0 | 0.11141 |
| 1546. | LOC642567 | XR_038054.1 | −1.22197896 | 0.561394485 | 6.83E−08 | 0.001055478 | 0 | 0.00603 |
| 1547. | SPG11 | NM_025137.3 | −1.22356765 | 0.295100761 | 3.20E−12 | 0.001743243 | 0 | 0.00918 |
| 1548. | ALDH3B1 | NM_000694.2 | −1.22406271 | −0.14825426 | 2.81E−11 | 0.123102729 | 0 | 0.26762 |
| 1549. | KIAA0240 | NM_015349.1 | −1.22507565 | −0.24824381 | 1.00E−12 | 0.004154924 | 0 | 0.01901 |
| 1550. | BCL3 | NM_005178.2 | −1.22811257 | −0.04482055 | 5.66E−12 | 0.601406976 | 0 | 0.76062 |
| 1551. | ARHGAP23 | XM_290799.7 | −1.2287201 | 0.001285621 | 1.57E−11 | 0.988672776 | 0 | 0.99512 |
| 1552. | EML3 | NM_153265.2 | −1.22888874 | −0.15256668 | 1.46E−12 | 0.066106231 | 0 | 0.1699 |
| 1553. | ZNF37A | NM_003421.1 | −1.23034477 | 0.20955543 | 1.34E−11 | 0.028646756 | 0 | 0.08938 |
| 1554. | ANGPT1 | NM_001146.3 | −1.23277337 | 0.222095158 | 3.26E−11 | 0.027771302 | 0 | 0.08724 |
| 1555. | C5orf62 | NM_032947.3 | −1.23335417 | −0.05705214 | 4.90E−12 | 0.505452995 | 0 | 0.68565 |
| 1556. | TCEAL4 | NM_001006936.1 | −1.2352506 | −0.34355105 | 8.90E−12 | 0.000822757 | 0 | 0.0049 |
| 1557. | CHES1 | NM_005197.2 | −1.23542276 | −0.42667702 | 6.19E−13 | 1.60E−05 | 0 | 0.00018 |
| 1558. | TWIST1 | NM_000474.3 | −1.23546753 | 0.474840471 | 3.41E−12 | 1.35E−05 | 0 | 0.00016 |
| 1559. | ATP2B4 | NM_001001396.1 | −1.24108982 | −0.41928272 | 1.39E−11 | 0.000152354 | 0 | 0.00117 |
| 1560. | EDN1 | NM_001955.2 | −1.2421427 | 0.553573183 | 2.25E−12 | 1.37E−06 | 0 | 2.00E−05 |
| 1561. | NFE2L3 | NM_004289.5 | −1.24368359 | 1.282660919 | 5.03E−12 | 2.88E−12 | 0 | 0 |
| 1562. | RIN2 | NM_018993.2 | −1.24377483 | −0.12732257 | 1.26E−11 | 0.170097728 | 0 | 0.33581 |
| 1563. | BAX | NM_138765.2 | −1.24393204 | −0.09455657 | 1.82E−10 | 0.374229123 | 0 | 0.56948 |
| 1564. | UBL4A | NM_014235.3 | −1.24473014 | −0.05485005 | 2.62E−12 | 0.51132399 | 0 | 0.68951 |
| 1565. | TMEM42 | NM_144638.1 | −1.24504816 | −0.41076142 | 1.21E−12 | 4.35E−05 | 0 | 0.00041 |
| 1566. | TAP2 | NM_018833.2 | −1.24679634 | 1.347745454 | 1.74E−11 | 4.29E−12 | 0 | 0 |
| 1567. | IL18BP | NM_173042.2 | −1.24818958 | 1.396028376 | 5.58E−11 | 7.59E−12 | 0 | 0 |
| 1568. | KIAA1751 | NM_001080484.1 | −1.24999314 | 0.721013137 | 1.17E−11 | 1.19E−07 | 0 | 0 |
| 1569. | PDGFRB | NM_002609.3 | −1.25003443 | −0.58162796 | 7.61E−12 | 1.93E−06 | 0 | 3.00E−05 |
| 1570. | ZNF362 | NM_152493.2 | −1.25018261 | −0.41738427 | 4.87E−12 | 9.15E−05 | 0 | 0.00076 |
| 1571. | FTSJD2 | NM_015050.2 | −1.25058376 | 0.458889696 | 5.64E−12 | 3.37E−05 | 0 | 0.00033 |
| 1572. | CNN2 | NM_201277.1 | −1.25059176 | −0.62721965 | 5.76E−11 | 3.28E−06 | 0 | 5.00E−05 |
| 1573. | KAT2B | NM_003884.4 | −1.25085462 | −0.24666958 | 2.93E−12 | 0.00758648 | 0 | 0.03119 |
| 1574. | PARP8 | NM_024615.2 | −1.25290424 | 0.572054226 | 1.04E−12 | 5.29E−07 | 0 | 1.00E−05 |
| 1575. | DAAM2 | NM_015345.2 | −1.2552281 | −0.05691031 | 1.89E−11 | 0.544533117 | 0 | 0.71697 |
| 1576. | MAP4K2 | NM_004579.2 | −1.25577347 | −0.24514143 | 2.87E−13 | 0.00329692 | 0 | 0.01566 |
| 1577. | MTMR11 | NM_181873.2 | −1.25596886 | 0.448810783 | 1.48E−12 | 1.90E−05 | 0 | 2.00E−04 |
| 1578. | BCL6 | NM_001706.2 | −1.25882628 | 0.364052652 | 1.15E−11 | 0.000653022 | 0 | 0.00403 |
| 1579. | AK3 | NM_016282.2 | −1.25911346 | −0.42562754 | 6.30E−13 | 2.11E−05 | 0 | 0.00022 |
| 1580. | TTC39B | NM_152574.1 | −1.26035136 | 1.231073808 | 3.07E−11 | 4.66E−11 | 0 | 0 |
| 1581. | KLF9 | NM_001206.2 | −1.26060289 | −0.05122193 | 2.18E−13 | 0.488273883 | 0 | 0.67176 |
| 1582. | COPS8 | NM_006710.4 | −1.26063636 | 0.141895302 | 2.56E−11 | 0.14816821 | 0 | 0.30536 |
| 1583. | ASAM | NM_024769.2 | −1.26092249 | 0.021724338 | 2.37E−12 | 0.795291685 | 0 | 0.88975 |
| 1584. | INHBE | NM_031479.3 | −1.26114268 | −0.48724046 | 3.41E−10 | 0.00025202 | 0 | 0.00181 |
| 1585. | RFTN1 | NM_015150.1 | −1.26124118 | −0.40465466 | 9.94E−13 | 5.32E−05 | 0 | 0.00049 |
| 1586. | GPX1 | NM_201397.1 | −1.26164419 | 0.316305966 | 1.44E−11 | 0.002512932 | 0 | 0.01244 |
| 1587. | ANKRA2 | NM_023039.2 | −1.26244562 | 0.338540244 | 1.67E−12 | 0.000485656 | 0 | 0.00316 |
| 1588. | CLDN23 | NM_194284.2 | −1.26399949 | 1.176748203 | 3.29E−11 | 1.16E−10 | 0 | 0 |
| 1589. | KAT2A | NM_021078.2 | −1.26435612 | 0.060322015 | 2.91E−13 | 0.423975868 | 0 | 0.61575 |
| 1590. | SLC3A2 | NM_001013251.1 | −1.26545795 | 0.646988058 | 2.08E−12 | 1.74E−07 | 0 | 0 |
| 1591. | MT1G | NM_005950.1 | −1.26641147 | 0.254910746 | 1.51E−09 | 0.046262194 | 0 | 0.12953 |
| 1592. | LRDD | NM_018494.3 | −1.26748931 | 0.006338396 | 1.29E−10 | 0.951967571 | 0 | 0.97732 |
| 1593. | CCDC92 | NM_025140.1 | −1.26797415 | −0.31152889 | 6.83E−12 | 0.002115927 | 0 | 0.01081 |
| 1594. | MMP7 | NM_002423.3 | −1.2679778 | 0.212705137 | 1.56E−11 | 0.032117689 | 0 | 0.09767 |
| 1595. | HOXA10 | NM_018951.3 | −1.26824789 | −0.07148051 | 9.17E−13 | 0.375767851 | 0 | 0.57122 |
| 1596. | LOC100129034 | XM_001720357.1 | −1.26934501 | −0.3388464 | 5.08E−11 | 0.002680652 | 0 | 0.01312 |
| 1597. | TNFAIP8 | NM_001077654.1 | −1.26961714 | 0.790314802 | 8.72E−12 | 2.81E−08 | 0 | 0 |
| 1598. | NNMT | NM_006169.2 | −1.27135745 | 0.155351948 | 1.98E−13 | 0.04551244 | 0 | 0.12794 |
| 1599. | TLR3 | NM_003265.2 | −1.27150188 | 1.667818088 | 3.74E−11 | 2.74E−13 | 0 | 0 |
| 1600. | ABI3BP | NM_015429.2 | −1.27210874 | −0.40694881 | 6.29E−13 | 4.11E−05 | 0 | 0.00039 |
| 1601. | RNF216 | NM_207111.2 | −1.27291066 | −0.27943523 | 9.26E−13 | 0.002160566 | 0 | 0.01099 |
| 1602. | NDUFA4L2 | NM_020142.3 | −1.27293073 | 0.189521586 | 1.12E−10 | 0.082270846 | 0 | 0.2002 |
| 1603. | ADARB1 | NM_001112.2 | −1.27392591 | −0.35663003 | 1.62E−12 | 0.000312879 | 0 | 0.00217 |
| 1604. | UBAP2L | NM_014847.2 | −1.2740099 | −0.14642379 | 3.69E−11 | 0.148118985 | 0 | 0.30531 |
| 1605. | GLIPR2 | NM_022343.2 | −1.27459398 | −0.7108052 | 1.96E−12 | 4.32E−08 | 0 | 0 |
| 1606. | MIR1978 | NR_031742.1 | −1.27530556 | 0.085704085 | 6.74E−13 | 0.285110377 | 0 | 0.47922 |
| 1607. | MR1 | NM_001531.1 | −1.27624432 | 0.675729769 | 5.92E−13 | 3.50E−08 | 0 | 0 |
| 1608. | LMO3 | NM_018640.3 | −1.27766577 | −1.05553487 | 2.28E−13 | 7.46E−12 | 0 | 0 |
| 1609. | FLJ41484 | XR_042107.1 | −1.28020815 | −0.09400577 | 6.73E−13 | 0.244088251 | 0 | 0.4309 |
| 1610. | OXTR | NM_000916.3 | −1.28131083 | −0.80945236 | 2.41E−12 | 7.02E−09 | 0 | 0 |
| 1611. | BATF2 | NM_138456.3 | −1.28133994 | 1.382835836 | 7.54E−12 | 1.90E−12 | 0 | 0 |
| 1612. | CPT1A | NM_001031847.1 | −1.28265842 | 1.626021912 | 1.08E−12 | 1.32E−14 | 0 | 0 |
| 1613. | YPEL3 | NM_031477.4 | −1.28323494 | −0.2724666 | 5.98E−11 | 0.013731293 | 0 | 0.05028 |
| 1614. | ALDH3A2 | NM_000382.2 | −1.28349337 | −0.19932451 | 7.36E−13 | 0.020201185 | 0 | 0.06808 |
| 1615. | SSH2 | NM_033389.2 | −1.28411793 | −0.31835469 | 6.22E−12 | 0.001883032 | 0 | 0.0098 |
| 1616. | SLC2A6 | NM_017585.2 | −1.28437053 | 0.378482079 | 3.37E−12 | 0.000279566 | 0 | 0.00197 |
| 1617. | ECH1 | NM_001398.2 | −1.28639716 | −0.34546234 | 2.28E−11 | 0.001768614 | 0 | 0.0093 |
| 1618. | HSD3B7 | NM_025193.2 | −1.28658642 | 0.103366864 | 9.65E−12 | 0.270186716 | 0 | 0.46164 |
| 1619. | DYRK4 | NM_003845.1 | −1.28717119 | −0.05162009 | 1.31E−11 | 0.58398072 | 0 | 0.74633 |
| 1620. | SIRT5 | NM_012241.2 | −1.28868154 | 0.21980702 | 5.06E−11 | 0.040381201 | 0 | 0.11698 |
| 1621. | SIL1 | NM_001037633.1 | −1.29059615 | −0.09340813 | 3.83E−10 | 0.416952518 | 0 | 0.60971 |
| 1622. | SSBP2 | NM_012446.2 | −1.29355571 | 0.350190938 | 8.67E−13 | 0.000310366 | 0 | 0.00215 |
| 1623. | SLC26A6 | NM_134426.2 | −1.29360831 | 0.136065221 | 2.18E−11 | 0.171482074 | 0 | 0.33766 |
| 1624. | BCL2L13 | NM_015367.2 | −1.2937724 | 0.673244382 | 6.85E−12 | 3.57E−07 | 0 | 1.00E−05 |
| 1625. | BTN3A1 | NM_007048.4 | −1.29603027 | 0.543279714 | 2.10E−12 | 3.04E−06 | 0 | 5.00E−05 |
| 1626. | WDR81 | NM_152348.1 | −1.297512 | 0.005641021 | 1.81E−10 | 0.959046599 | 0 | 0.98125 |
| 1627. | NRCAM | NM_005010.3 | −1.29976474 | −0.11197225 | 1.30E−12 | 0.189586356 | 0 | 0.36299 |
| 1628. | FTL | NM_000146.3 | −1.29977216 | 0.000160421 | 2.24E−11 | 0.998690037 | 0 | 0.99941 |
| 1629. | AXL | NM_021913.2 | −1.30144788 | 0.099042211 | 6.95E−11 | 0.34787206 | 0 | 0.54459 |
| 1630. | HTATIP2 | NM_006410.3 | −1.30236259 | 0.519560176 | 8.40E−12 | 1.58E−05 | 0 | 0.00018 |
| 1631. | SUSD1 | NM_022486.3 | −1.30337034 | 0.513922476 | 1.21E−12 | 4.61E−06 | 0 | 6.00E−05 |
| 1632. | CIDEC | NM_022094.2 | −1.30550645 | −0.03097348 | 3.40E−12 | 0.726351736 | 0 | 0.84614 |
| 1633. | C6orf138 | NM_001013732.2 | −1.30560647 | 0.97958877 | 4.10E−13 | 7.28E−11 | 0 | 0 |
| 1634. | ZNF564 | NM_144976.2 | −1.30568726 | −0.10146929 | 3.40E−13 | 0.202342093 | 0 | 0.3789 |
| 1635. | TRIB3 | NM_021158.3 | −1.30713349 | 0.576140127 | 6.37E−11 | 1.89E−05 | 0 | 2.00E−04 |
| 1636. | CENTG2 | NM_014914.2 | −1.30782772 | 0.524880457 | 3.72E−12 | 8.26E−06 | 0 | 1.00E−04 |
| 1637. | KYNU | NM_003937.2 | −1.31136861 | 1.459677604 | 1.49E−11 | 2.16E−12 | 0 | 0 |
| 1638. | SIX2 | NM_016932.3 | −1.31183369 | −0.14765444 | 2.39E−13 | 0.065947869 | 0 | 0.16959 |
| 1639. | EBF3 | NM_001005463.1 | −1.3149055 | −0.55607247 | 1.17E−13 | 2.86E−07 | 0 | 1.00E−05 |
| 1640. | HOXC8 | NM_022658.3 | −1.3151235 | −0.25369686 | 2.67E−13 | 0.003504244 | 0 | 0.01648 |
| 1641. | HOXC4 | NM_014620.4 | −1.31705844 | −0.28598047 | 9.00E−13 | 0.002330636 | 0 | 0.01171 |
| 1642. | SIX1 | NM_005982.2 | −1.31890565 | 0.408410823 | 2.44E−12 | 0.000137543 | 0 | 0.00107 |
| 1643. | TRAM2 | NM_012288.3 | −1.32152945 | −0.17572653 | 8.43E−12 | 0.073320888 | 0 | 0.18351 |
| 1644. | KBTBD11 | NM_014867.1 | −1.32249933 | −0.52953994 | 2.04E−12 | 5.52E−06 | 0 | 7.00E−05 |
| 1645. | TMEM219 | NM_001083613.1 | −1.32737482 | 0.553093995 | 1.68E−13 | 4.76E−07 | 0 | 1.00E−05 |
| 1646. | PDXK | NM_003681.4 | −1.32939426 | −0.66103564 | 9.24E−13 | 1.35E−07 | 0 | 0 |
| 1647. | FTHL2 | NR_002200.1 | −1.32944895 | 0.372503922 | 1.34E−09 | 0.007456489 | 0 | 0.03074 |
| 1648. | OASL | NM_003733.2 | −1.3310586 | 1.741985948 | 4.39E−09 | 4.37E−11 | 0 | 0 |
| 1649. | NEURL1B | NM_001142651.1 | −1.33356876 | −0.32492145 | 3.50E−11 | 0.004559356 | 0 | 0.02051 |
| 1650. | COMT | NM_007310.1 | −1.33455371 | 0.198907421 | 1.03E−11 | 0.049152815 | 0 | 0.13566 |
| 1651. | PYCARD | NM_013258.3 | −1.33555746 | 0.423841029 | 2.99E−12 | 0.000119073 | 0 | 0.00095 |
| 1652. | OSBPL5 | NM_020896.2 | −1.33616498 | −0.066337 | 8.92E−12 | 0.489545976 | 0 | 0.67275 |
| 1653. | MICAL2 | NM_014632.2 | −1.33709412 | 0.007846246 | 7.63E−11 | 0.941939083 | 0 | 0.97186 |
| 1654. | ASTN2 | NM_198186.2 | −1.33716772 | −0.22246192 | 1.89E−12 | 0.018378017 | 0 | 0.06331 |
| 1655. | STAT6 | NM_003153.3 | −1.33735612 | 0.543277292 | 5.92E−13 | 1.81E−06 | 0 | 3.00E−05 |
| 1656. | ST3GAL4 | NM_006278.1 | −1.33916714 | −0.33464779 | 2.24E−11 | 0.003126343 | 0 | 0.01497 |
| 1657. | ACSM5 | NM_017888.2 | −1.33925951 | 0.052274036 | 1.13E−11 | 0.590865362 | 0 | 0.75205 |
| 1658. | LOC100133866 | XM_001719715.1 | −1.34044657 | −0.13452766 | 2.86E−12 | 0.145392177 | 0 | 0.30129 |
| 1659. | TGFBR3 | NM_003243.2 | −1.34123275 | 0.459930342 | 1.42E−13 | 6.27E−06 | 0 | 8.00E−05 |
| 1660. | CXCL2 | NM_002089.3 | −1.34255656 | −0.00014171 | 1.67E−12 | 0.998705957 | 0 | 0.99941 |
| 1661. | IL1R1 | NM_000877.2 | −1.34412575 | 0.29854828 | 2.57E−12 | 0.003066441 | 0 | 0.01472 |
| 1662. | RTN1 | NM_021136.2 | −1.34513809 | −0.05720283 | 1.53E−13 | 0.460039047 | 0 | 0.64836 |
| 1663. | MUSK | NM_005592.1 | −1.34833055 | 0.621728343 | 3.76E−11 | 7.27E−06 | 0 | 9.00E−05 |
| 1664. | TANC2 | NM_025185.3 | −1.34885142 | 0.152505822 | 5.80E−13 | 0.077349509 | 0 | 0.19121 |
| 1665. | A4GALT | NM_017436.4 | −1.34968348 | 0.247106452 | 3.48E−13 | 0.005691938 | 0 | 0.02459 |
| 1666. | LOC644423 | XM_930172.1 | −1.35186033 | 0.002355557 | 1.46E−11 | 0.981060488 | 0 | 0.9915 |
| 1667. | PTPRM | NM_002845.2 | −1.35229974 | 0.239150348 | 8.61E−13 | 0.009942472 | 0 | 0.03869 |
| 1668. | LNPEP | NM_175920.3 | −1.35302576 | 2.076552667 | 1.06E−11 | 3.91E−15 | 0 | 0 |
| 1669. | SCO2 | NM_005138.1 | −1.35315118 | −0.05621753 | 3.13E−13 | 0.487127801 | 0 | 0.67073 |
| 1670. | SLC7A11 | NM_014331.3 | −1.3550606 | 1.353933993 | 5.00E−12 | 5.07E−12 | 0 | 0 |
| 1671. | TMBIM1 | NM_022152.4 | −1.35648844 | 0.033681867 | 2.75E−12 | 0.711009202 | 0 | 0.83581 |
| 1672. | TCEAL3 | NM_032926.2 | −1.35654241 | −0.50350419 | 3.01E−13 | 3.76E−06 | 0 | 5.00E−05 |
| 1673. | TRIM47 | NM_033452.2 | −1.35992613 | 0.107549127 | 1.62E−12 | 0.232254719 | 0 | 0.41657 |
| 1674. | WISP1 | NM_003882.2 | −1.36026694 | −0.42293489 | 9.15E−13 | 7.24E−05 | 0 | 0.00063 |
| 1675. | TRIM21 | NM_003141.3 | −1.36120539 | 0.978702849 | 5.36E−14 | 2.22E−11 | 0 | 0 |
| 1676. | HLA-G | NM_002127.3 | −1.36158714 | 0.117153293 | 1.05E−12 | 0.185080548 | 0 | 0.35647 |
| 1677. | GMPPA | NM_205847.1 | −1.36274104 | −0.09236449 | 3.29E−12 | 0.32192772 | 0 | 0.51862 |
| 1678. | SERPINE2 | NM_006216.2 | −1.36686711 | 0.204965168 | 3.95E−12 | 0.037947323 | 0 | 0.1113 |
| 1679. | TLCD1 | NM_138463.2 | −1.36823341 | −0.41257407 | 4.66E−12 | 0.000265336 | 0 | 0.00189 |
| 1680. | WASF2 | NM_006990.2 | −1.3713366 | 0.172240006 | 6.09E−11 | 0.125730924 | 0 | 0.27174 |
| 1681. | ABR | NM_001092.3 | −1.37442122 | −0.08824512 | 6.84E−14 | 0.248952398 | 0 | 0.43679 |
| 1682. | APCDD1L | NM_153360.1 | −1.37521817 | 0.283989552 | 1.92E−13 | 0.001768998 | 0 | 0.0093 |
| 1683. | DFNA5 | NM_004403.2 | −1.37676256 | −0.03364312 | 1.63E−12 | 0.707506944 | 0 | 0.83362 |
| 1684. | PSTPIP2 | NM_024430.2 | −1.38071815 | 0.475342336 | 1.46E−12 | 2.95E−05 | 0 | 3.00E−04 |
| 1685. | BIRC3 | NM_182962.1 | −1.38133805 | 0.762030268 | 2.66E−10 | 2.95E−06 | 0 | 4.00E−05 |
| 1686. | SGCD | NM_172244.2 | −1.38361246 | 0.134345928 | 3.33E−11 | 0.215713603 | 0 | 0.3962 |
| 1687. | EGFR | NM_005228.3 | −1.38570441 | 1.211540136 | 7.40E−13 | 8.49E−12 | 0 | 0 |
| 1688. | NPTX2 | NM_002523.1 | −1.38607355 | 0.12199302 | 2.44E−13 | 0.144261408 | 0 | 0.29944 |
| 1689. | DUSP10 | NM_144729.1 | −1.3898476 | −0.36589885 | 3.40E−13 | 0.000245293 | 0 | 0.00177 |
| 1690. | RELB | NM_006509.2 | −1.39149325 | 0.291381817 | 1.06E−13 | 0.001195706 | 0 | 0.00667 |
| 1691. | ZNF395 | NM_018660.2 | −1.39214173 | 0.258424553 | 2.92E−12 | 0.011163004 | 0 | 0.04241 |
| 1692. | ANKFY1 | NM_016376.3 | −1.39551572 | 0.094094818 | 1.02E−12 | 0.294195397 | 0 | 0.48882 |
| 1693. | MCEE | NM_032601.2 | −1.39785582 | −0.34103882 | 5.73E−14 | 0.000199452 | 0 | 0.00147 |
| 1694. | CYGB | NM_134268.3 | −1.39837281 | 0.157034411 | 1.02E−12 | 0.087977277 | 0 | 0.21022 |
| 1695. | SUSD2 | NM_019601.3 | −1.39843283 | −2.02214113 | 6.00E−12 | 6.59E−15 | 0 | 0 |
| 1696. | ARHGEF19 | NM_153213.3 | −1.40022764 | 0.110336957 | 3.26E−13 | 0.195395087 | 0 | 0.37013 |
| 1697. | STEAP3 | NM_018234.2 | −1.40487084 | −0.29100423 | 1.77E−13 | 0.001663157 | 0 | 0.00883 |
| 1698. | RRAS | NM_006270.3 | −1.40541909 | −0.19078881 | 1.10E−13 | 0.023869464 | 0 | 0.07777 |
| 1699. | DDB2 | NM_000107.1 | −1.40753645 | 0.107266526 | 7.53E−13 | 0.230075579 | 0 | 0.41363 |
| 1700. | GALK1 | NM_000154.1 | −1.41068025 | −0.5287698 | 2.80E−12 | 1.61E−05 | 0 | 0.00018 |
| 1701. | OCEL1 | NM_024578.1 | −1.4121561 | −0.09909807 | 2.45E−11 | 0.358256689 | 0 | 0.55434 |
| 1702. | C8orf13 | NM_053279.1 | −1.4123943 | 0.423496703 | 2.09E−13 | 4.36E−05 | 0 | 0.00041 |
| 1703. | PLIN2 | NM_001122.2 | −1.41334198 | 0.610730944 | 1.28E−12 | 1.38E−06 | 0 | 2.00E−05 |
| 1704. | PHF15 | NM_015288.4 | −1.41505052 | 0.255681623 | 2.00E−12 | 0.011596219 | 0 | 0.04373 |
| 1705. | LOC653879 | XM_936226.1 | −1.41570984 | 0.326110603 | 5.25E−14 | 0.000342368 | 0 | 0.00234 |
| 1706. | ZBTB4 | NM_020899.2 | −1.41747596 | 0.002479184 | 3.05E−14 | 0.97337881 | 0 | 0.98766 |
| 1707. | GRINA | NM_000837.1 | −1.42662154 | 0.468347683 | 4.64E−12 | 0.00010699 | 0 | 0.00087 |
| 1708. | PLA2G4C | NM_003706.1 | −1.42905108 | 0.074031317 | 5.43E−14 | 0.343549283 | 0 | 0.54053 |
| 1709. | BHLHB2 | NM_003670.1 | −1.43040915 | 0.622162809 | 2.30E−11 | 1.08E−05 | 0 | 0.00013 |
| 1710. | FOXQ1 | NM_033260.3 | −1.43194603 | 0.640258886 | 1.41E−11 | 5.28E−06 | 0 | 7.00E−05 |
| 1711. | IMPA2 | NM_014214.1 | −1.43667021 | −0.34113829 | 7.81E−13 | 0.001022693 | 0 | 0.00587 |
| 1712. | LOC728431 | XM_001132105.2 | −1.44016997 | 0.454875198 | 1.74E−12 | 9.03E−05 | 0 | 0.00075 |
| 1713. | IGFBP5 | NM_000599.2 | −1.4407583 | 0.178012333 | 1.93E−12 | 0.071191885 | 0 | 0.17968 |
| 1714. | AHNAK | NM_001620.1 | −1.44089244 | 0.16302896 | 1.43E−11 | 0.133971567 | 0 | 0.28394 |
| 1715. | MYH13 | NM_003802.2 | −1.44106374 | −1.54057835 | 2.59E−12 | 7.65E−13 | 0 | 0 |
| 1716. | PLXNB1 | NM_002673.3 | −1.44204532 | −0.09842679 | 3.68E−12 | 0.321593695 | 0 | 0.51821 |
| 1717. | MT2A | NM_005953.2 | −1.44318324 | 0.084693759 | 8.45E−14 | 0.295950147 | 0 | 0.49062 |
| 1718. | SPTLC3 | NM_018327.2 | −1.44438926 | 0.242808954 | 5.24E−14 | 0.005053828 | 0 | 0.02229 |
| 1719. | IRAK2 | NM_001570.3 | −1.44684212 | 0.511612169 | 1.80E−13 | 4.96E−06 | 0 | 7.00E−05 |
| 1720. | CCDC8 | NM_032040.2 | −1.45318072 | −0.09224991 | 3.24E−12 | 0.352429295 | 0 | 0.54879 |
| 1721. | ASNS | NM_133436.1 | −1.4541773 | 0.71333254 | 1.05E−12 | 1.85E−07 | 0 | 0 |
| 1722. | ATL3 | NM_015459.3 | −1.45534869 | 1.351820113 | 7.05E−12 | 2.64E−11 | 0 | 0 |
| 1723. | PLEKHA4 | NM_020904.1 | −1.45689332 | 1.330489225 | 5.74E−13 | 3.01E−12 | 0 | 0 |
| 1724. | PPP3CC | NM_005605.3 | −1.45719007 | 0.256236482 | 5.52E−14 | 0.003710913 | 0 | 0.01731 |
| 1725. | TRIP6 | NM_003302.2 | −1.45738819 | 0.003437988 | 1.53E−13 | 0.967080994 | 0 | 0.98535 |
| 1726. | LOC387763 | XM_941665.2 | −1.4595833 | 0.204766293 | 4.83E−14 | 0.015687493 | 0 | 0.05583 |
| 1727. | CYP26B1 | NM_019885.2 | −1.46093263 | 0.414869119 | 7.55E−14 | 4.33E−05 | 0 | 0.00041 |
| 1728. | LIMA1 | NM_016357.3 | −1.4628141 | 0.256513096 | 6.31E−13 | 0.009423844 | 0 | 0.03711 |
| 1729. | AGTRAP | NM_001040196.1 | −1.46690619 | 0.615176031 | 1.37E−13 | 3.66E−07 | 0 | 1.00E−05 |
| 1730. | RUSC1 | NM_014328.2 | −1.47032189 | 0.034636238 | 4.91E−14 | 0.66207751 | 0 | 0.80364 |
| 1731. | P2RX6 | NM_005446.3 | −1.47477301 | −0.74322828 | 1.15E−12 | 1.32E−07 | 0 | 0 |
| 1732. | RALGDS | NM_006266.2 | −1.47507059 | 0.068597962 | 9.07E−13 | 0.463187756 | 0 | 0.65109 |
| 1733. | C14orf4 | NM_024496.2 | −1.47638749 | 0.053488086 | 1.48E−13 | 0.527623056 | 0 | 0.70308 |
| 1734. | PSME2 | NM_002818.2 | −1.47714939 | 0.430428021 | 9.35E−14 | 3.65E−05 | 0 | 0.00036 |
| 1735. | PTPRU | NM_005704.3 | −1.47716192 | 0.26117597 | 6.03E−13 | 0.008797949 | 0 | 0.03516 |
| 1736. | GBP4 | NM_052941.3 | −1.47784861 | 2.337304616 | 8.12E−10 | 2.29E−13 | 0 | 0 |
| 1737. | RGS20 | NM_170587.1 | −1.47833808 | 0.086519728 | 8.38E−12 | 0.414837927 | 0 | 0.60778 |
| 1738. | RRBP1 | NM_001042576.1 | −1.48268558 | 0.099040674 | 7.19E−13 | 0.289587832 | 0 | 0.48427 |
| 1739. | PARP3 | NM_005485.3 | −1.48285466 | 0.160642711 | 6.43E−15 | 0.034316885 | 0 | 0.10284 |
| 1740. | MIOS | NM_019005.3 | −1.48456566 | 0.123769389 | 1.11E−13 | 0.148663341 | 0 | 0.30617 |
| 1741. | DNAJB2 | NM_006736.5 | −1.48817735 | −0.01635434 | 1.84E−12 | 0.866678588 | 0 | 0.93058 |
| 1742. | ABCC3 | NM_003786.2 | −1.49080054 | 0.134908152 | 2.08E−13 | 0.130736985 | 0 | 0.27892 |
| 1743. | MYBPHL | NM_001010985.1 | −1.49245365 | −0.52050028 | 1.51E−13 | 5.26E−06 | 0 | 7.00E−05 |
| 1744. | CABC1 | NM_020247.4 | −1.49405141 | −0.17570193 | 1.82E−12 | 0.083852085 | 0 | 0.20301 |
| 1745. | IRAK3 | NM_007199.1 | −1.49963531 | 1.053332087 | 2.13E−13 | 1.24E−10 | 0 | 0 |
| 1746. | MMP3 | NM_002422.3 | −1.50941637 | 0.283943777 | 3.79E−13 | 0.004851214 | 0 | 0.02158 |
| 1747. | NFKB1 | NM_003998.2 | −1.5124906 | 0.055460651 | 6.37E−15 | 0.450306786 | 0 | 0.63957 |
| 1748. | RBM43 | NM_198557.2 | −1.51695343 | 0.321509124 | 5.92E−12 | 0.006055392 | 0 | 0.0259 |
| 1749. | LOC389386 | XR_037483.1 | −1.52070072 | 1.370105547 | 1.17E−12 | 7.75E−12 | 0 | 0 |
| 1750. | CEBPD | NM_005195.3 | −1.52086996 | 0.159586563 | 1.08E−14 | 0.044768075 | 0 | 0.12632 |
| 1751. | PDK4 | NM_002612.3 | −1.52095915 | 0.026993134 | 1.11E−10 | 0.829383614 | 0 | 0.90954 |
| 1752. | DDIT4 | NM_019058.2 | −1.52174691 | 0.346018256 | 1.85E−13 | 0.000745673 | 0 | 0.00451 |
| 1753. | CA9 | NM_001216.1 | −1.52569921 | 0.523674918 | 1.44E−13 | 6.27E−06 | 0 | 8.00E−05 |
| 1754. | MT1A | NM_005946.2 | −1.52589374 | −0.12115771 | 3.49E−13 | 0.193723242 | 0 | 0.36827 |
| 1755. | COL8A1 | NM_020351.2 | −1.52684464 | 0.469023873 | 1.78E−13 | 2.94E−05 | 0 | 3.00E−04 |
| 1756. | TRNP1 | NM_001013642.2 | −1.52696476 | 0.007844311 | 8.13E−14 | 0.925910254 | 0 | 0.9642 |
| 1757. | VEZF1 | NM_007146.2 | −1.52847706 | −0.68759888 | 4.95E−14 | 5.51E−08 | 0 | 0 |
| 1758. | TRAF3IP2 | NM_147686.1 | −1.52911489 | 0.729932932 | 2.95E−14 | 1.38E−08 | 0 | 0 |
| 1759. | PRKCD | NM_006254.3 | −1.53061669 | 0.34903041 | 1.31E−14 | 0.000176699 | 0 | 0.00133 |
| 1760. | TCP11L1 | NM_018393.2 | −1.53218998 | 0.781612214 | 9.93E−14 | 1.35E−08 | 0 | 0 |
| 1761. | SLC25A28 | NM_031212.3 | −1.53435397 | 0.725087166 | 2.24E−12 | 5.93E−07 | 0 | 1.00E−05 |
| 1762. | HOXC6 | NM_004503.3 | −1.53945768 | −0.19267894 | 5.83E−15 | 0.015807586 | 0 | 0.05619 |
| 1763. | FAM175A | NM_139076.2 | −1.53954297 | 0.266657648 | 1.48E−12 | 0.013518962 | 0 | 0.04962 |
| 1764. | IDH3B | NM_006899.2 | −1.54157745 | 0.071174249 | 2.54E−14 | 0.378507709 | 0 | 0.57405 |
| 1765. | ZFP36L2 | NM_006887.3 | −1.54491503 | 0.733756556 | 1.85E−14 | 1.00E−08 | 0 | 0 |
| 1766. | ZC3H12A | NM_025079.1 | −1.54576097 | 0.542550698 | 2.07E−14 | 1.11E−06 | 0 | 2.00E−05 |
| 1767. | ISCU | NM_213595.1 | −1.54745085 | 0.039751741 | 9.00E−13 | 0.683731023 | 0 | 0.81833 |
| 1768. | APOL3 | NM_145641.1 | −1.54756883 | 0.977671581 | 1.51E−13 | 5.63E−10 | 0 | 0 |
| 1769. | LAP3 | NM_015907.2 | −1.55248459 | 1.401703664 | 7.15E−13 | 4.60E−12 | 0 | 0 |
| 1770. | CLDN15 | NM_138429.1 | −1.55417589 | −0.37181123 | 5.22E−12 | 0.002326424 | 0 | 0.01169 |
| 1771. | PHLDA3 | NM_012396.3 | −1.5571977 | 0.195775776 | 5.43E−14 | 0.029014612 | 0 | 0.09029 |
| 1772. | NT5E | NM_002526.1 | −1.55814764 | 0.488962102 | 4.51E−15 | 1.72E−06 | 0 | 3.00E−05 |
| 1773. | TNS3 | NM_022748.10 | −1.560059 | −0.16482657 | 1.12E−14 | 0.043851492 | 0 | 0.12441 |
| 1774. | SLC22A18 | NM_002555.3 | −1.56048525 | −0.05234579 | 8.35E−13 | 0.593824828 | 0 | 0.75454 |
| 1775. | RCAN1 | NM_203418.1 | −1.56643139 | 0.991653906 | 1.01E−13 | 3.73E−10 | 0 | 0 |
| 1776. | ESPNL | NM_194312.1 | −1.56733436 | −0.29565305 | 1.05E−13 | 0.002820345 | 0 | 0.01374 |
| 1777. | STC1 | NM_003155.2 | −1.57082588 | 0.813221894 | 2.70E−14 | 3.39E−09 | 0 | 0 |
| 1778. | NFKBIZ | NM_001005474.1 | −1.57178323 | −0.26442137 | 8.50E−13 | 0.013437646 | 0 | 0.04939 |
| 1779. | ARID3A | NM_005224.2 | −1.57277746 | 0.286710916 | 1.34E−13 | 0.004049243 | 0 | 0.0186 |
| 1780. | BTG2 | NM_006763.2 | −1.57279598 | −0.12418703 | 6.20E−14 | 0.15768225 | 0 | 0.31881 |
| 1781. | PRDM1 | NM_001198.2 | −1.5732617 | −0.09376117 | 3.19E−13 | 0.322435081 | 0 | 0.51912 |
| 1782. | TMEM45A | NM_018004.1 | −1.57441733 | 0.412836488 | 1.65E−12 | 0.000597758 | 0 | 0.00375 |
| 1783. | FHL2 | NM_201555.1 | −1.58022635 | −0.08794467 | 5.66E−14 | 0.310783791 | 0 | 0.50637 |
| 1784. | SESN1 | NM_014454.1 | −1.58102515 | −0.1132538 | 8.26E−14 | 0.204563372 | 0 | 0.38176 |
| 1785. | SLC16A3 | NM_004207.2 | −1.58160744 | 0.487263538 | 4.91E−14 | 1.20E−05 | 0 | 0.00014 |
| 1786. | MYF5 | NM_005593.1 | −1.58718203 | 0.290969968 | 1.14E−11 | 0.01861409 | 0 | 0.064 |
| 1787. | TNFAIP2 | NM_006291.2 | −1.59343131 | 0.057125441 | 3.22E−13 | 0.548671707 | 0 | 0.7201 |
| 1788. | PSMB10 | NM_002801.2 | −1.59971337 | 0.746410983 | 2.40E−13 | 1.16E−07 | 0 | 0 |
| 1789. | CYP27A1 | NM_000784.2 | −1.59996322 | −0.05979732 | 1.11E−13 | 0.508262417 | 0 | 0.68753 |
| 1790. | FPR1 | NM_002029.3 | −1.60285891 | 0.932695249 | 8.65E−15 | 1.65E−10 | 0 | 0 |
| 1791. | MSC | NM_005098.3 | −1.60544476 | 0.252937939 | 6.80E−14 | 0.008646886 | 0 | 0.03473 |
| 1792. | ERAP2 | NM_022350.2 | −1.60546764 | 0.765755439 | 7.47E−14 | 3.11E−08 | 0 | 0 |
| 1793. | GPX8 | NM_001008397.2 | −1.60600685 | −0.33210501 | 1.14E−14 | 0.000443653 | 0 | 0.00291 |
| 1794. | ZFP90 | NM_133458.2 | −1.60888346 | −0.19729638 | 4.32E−14 | 0.030853859 | 0 | 0.09462 |
| 1795. | GFPT2 | NM_005110.1 | −1.61161512 | 0.049446341 | 2.15E−14 | 0.552652522 | 0 | 0.72356 |
| 1796. | FUCA1 | NM_000147.3 | −1.61457948 | 0.29086749 | 4.65E−15 | 0.001031226 | 0 | 0.00591 |
| 1797. | ADM | NM_001124.1 | −1.61909121 | 0.636719139 | 5.65E−15 | 7.36E−08 | 0 | 0 |
| 1798. | C18orf56 | NM_001012716.1 | −1.61938835 | 0.212638271 | 5.48E−14 | 0.023354241 | 0 | 0.0764 |
| 1799. | BTN3A3 | NM_197974.1 | −1.62006729 | 0.401899253 | 1.06E−13 | 0.000236524 | 0 | 0.00171 |
| 1800. | PDPN | NM_001006625.1 | −1.62289071 | −0.29015177 | 1.44E−12 | 0.01108206 | 0 | 0.04217 |
| 1801. | CSF3 | NM_000759.2 | −1.62631994 | 0.812630056 | 6.53E−15 | 1.73E−09 | 0 | 0 |
| 1802. | KRT17 | NM_000422.1 | −1.62634499 | 0.458485309 | 4.13E−14 | 3.21E−05 | 0 | 0.00032 |
| 1803. | C1RL | NM_016546.1 | −1.62653273 | −0.15887921 | 1.02E−12 | 0.13443148 | 0 | 0.28452 |
| 1804. | ID3 | NM_002167.2 | −1.6266375 | −0.07116852 | 2.28E−12 | 0.511148191 | 0 | 0.68941 |
| 1805. | NRXN2 | NM_138734.1 | −1.62710093 | −0.24438346 | 1.63E−14 | 0.007152991 | 0 | 0.02974 |
| 1806. | PTGES | NM_004878.3 | −1.63236485 | 0.31159722 | 8.37E−11 | 0.027177946 | 0 | 0.08579 |
| 1807. | RBCK1 | NM_031229.2 | −1.63564018 | 0.326034854 | 3.82E−14 | 0.001137043 | 0 | 0.0064 |
| 1808. | DDX60L | NM_001012967.1 | −1.63756912 | 0.657799485 | 2.88E−14 | 2.01E−07 | 0 | 0 |
| 1809. | PCTK3 | NM_212503.1 | −1.64072889 | 0.593223585 | 3.33E−13 | 5.74E−06 | 0 | 8.00E−05 |
| 1810. | IFIT5 | NM_012420.1 | −1.64540795 | −0.2831098 | 1.18E−14 | 0.002318567 | 0 | 0.01167 |
| 1811. | PLXNB2 | NM_012401.2 | −1.64908391 | 0.203132494 | 4.65E−14 | 0.030785988 | 0 | 0.09449 |
| 1812. | HCG4 | NR_002139.1 | −1.65084865 | 0.683826828 | 6.23E−14 | 2.35E−07 | 0 | 1.00E−05 |
| 1813. | MOV10 | NM_020963.2 | −1.65804038 | 0.686885229 | 1.41E−14 | 6.97E−08 | 0 | 0 |
| 1814. | KLF6 | NM_001008490.1 | −1.65904637 | 0.641898836 | 3.14E−14 | 3.76E−07 | 0 | 1.00E−05 |
| 1815. | RTTN | NM_173630.2 | −1.66298166 | 0.00938669 | 3.31E−14 | 0.914624753 | 0 | 0.95851 |
| 1816. | SERPING1 | NM_001032295.1 | −1.66774893 | 1.213582723 | 1.68E−14 | 5.89E−12 | 0 | 0 |
| 1817. | TNFRSF1A | NM_001065.2 | −1.67171265 | 0.272663339 | 8.11E−14 | 0.007323871 | 0 | 0.03031 |
| 1818. | SIX5 | NM_175875.3 | −1.67303085 | −0.36144719 | 7.93E−15 | 0.000235471 | 0 | 0.00171 |
| 1819. | DCN | NM_001920.3 | −1.67340671 | 0.264127506 | 4.46E−15 | 0.003041081 | 0 | 0.01462 |
| 1820. | LOC392437 | XR_037197.1 | −1.67432231 | 0.113041135 | 1.55E−14 | 0.191590462 | 0 | 0.36551 |
| 1821. | FAM110B | NM_147189.2 | −1.67457122 | −0.7651157 | 3.81E−14 | 3.46E−08 | 0 | 0 |
| 1822. | TNFRSF6B | NM_032945.2 | −1.67781723 | 0.009249704 | 6.69E−13 | 0.92905655 | 0 | 0.96585 |
| 1823. | HAS2 | NM_005328.1 | −1.67894462 | 0.691029466 | 4.16E−14 | 1.87E−07 | 0 | 0 |
| 1824. | XBP1 | NM_001079539.1 | −1.67922842 | 0.335668137 | 1.50E−14 | 0.000706062 | 0 | 0.00431 |
| 1825. | IL32 | NM_001012636.1 | −1.6806731 | 0.061122235 | 8.91E−15 | 0.461882443 | 0 | 0.64986 |
| 1826. | ZNF337 | NM_015655.2 | −1.69290143 | −0.04355874 | 8.45E−14 | 0.642972423 | 0 | 0.79019 |
| 1827. | NINJ1 | NM_004148.3 | −1.69423643 | 0.210694559 | 8.45E−15 | 0.018307905 | 0 | 0.06309 |
| 1828. | SQSTM1 | NM_003900.3 | −1.69542975 | 0.177118023 | 8.07E−15 | 0.042710085 | 0 | 0.12206 |
| 1829. | TCEA3 | NM_003196.1 | −1.70592123 | −0.12057791 | 5.05E−14 | 0.198902117 | 0 | 0.37451 |
| 1830. | MAOA | NM_000240.2 | −1.7102799 | −0.37585533 | 1.72E−13 | 0.000982147 | 0 | 0.00569 |
| 1831. | IFITM2 | NM_006435.2 | −1.7127993 | 0.006726498 | 5.54E−14 | 0.942150711 | 0 | 0.97189 |
| 1832. | MOCOS | NM_017947.1 | −1.71616703 | 0.417652421 | 4.47E−14 | 0.000177947 | 0 | 0.00134 |
| 1833. | TSC22D3 | NM_004089.3 | −1.72924291 | 0.200185627 | 1.14E−12 | 0.081009325 | 0 | 0.19791 |
| 1834. | GAS1 | NM_002048.1 | −1.73194473 | 0.007048827 | 4.70E−13 | 0.946577005 | 0 | 0.97406 |
| 1835. | RTKN | NM_033046.2 | −1.73436817 | −0.26783955 | 1.27E−13 | 0.012112938 | 0 | 0.04535 |
| 1836. | MUC1 | NM_001044390.1 | −1.73973553 | 0.350778496 | 1.01E−12 | 0.004324357 | 0 | 0.01964 |
| 1837. | RHBDF2 | NM_024599.3 | −1.74118145 | 0.381476507 | 4.86E−13 | 0.001642917 | 0 | 0.00874 |
| 1838. | PPAP2B | NM_177414.1 | −1.74179567 | 0.058853912 | 7.93E−15 | 0.491185271 | 0 | 0.67383 |
| 1839. | CNTNAP1 | NM_003632.1 | −1.74399554 | 0.148636651 | 1.49E−15 | 0.06775075 | 0 | 0.17309 |
| 1840. | HES4 | NM_021170.2 | −1.75076048 | 1.068396514 | 9.76E−14 | 6.79E−10 | 0 | 0 |
| 1841. | CRISPLD2 | NM_031476.2 | −1.75178737 | −0.45004083 | 5.63E−15 | 2.73E−05 | 0 | 0.00028 |
| 1842. | FKBP5 | NM_004117.2 | −1.75752416 | −0.10271459 | 6.57E−15 | 0.234812689 | 0 | 0.41952 |
| 1843. | CABYR | NM_153768.1 | −1.75759863 | −0.04313195 | 3.71E−14 | 0.644015484 | 0 | 0.79107 |
| 1844. | BTN3A2 | NM_007047.3 | −1.75860531 | 0.335634978 | 1.70E−15 | 0.000362323 | 0 | 0.00245 |
| 1845. | VASN | NM_138440.2 | −1.76049327 | 0.287659732 | 5.70E−15 | 0.002569233 | 0 | 0.01267 |
| 1846. | ZFHX3 | NM_006885.3 | −1.76153211 | 0.502851822 | 4.06E−15 | 5.76E−06 | 0 | 8.00E−05 |
| 1847. | ITPRIP | NM_033397.2 | −1.76269453 | 0.551625861 | 4.04E−14 | 8.59E−06 | 0 | 0.00011 |
| 1848. | SHISA5 | NM_016479.3 | −1.76302747 | −0.27352544 | 6.92E−16 | 0.001575872 | 0 | 0.00844 |
| 1849. | GSDMD | NM_024736.5 | −1.76317457 | 0.573835839 | 1.59E−14 | 2.63E−06 | 0 | 4.00E−05 |
| 1850. | MSI2 | NM_138962.2 | −1.76336708 | 1.326780914 | 3.14E−15 | 6.31E−13 | 0 | 0 |
| 1851. | TNFSF13B | NM_006573.3 | −1.76340877 | 2.189714333 | 1.09E−12 | 1.97E−14 | 0 | 0 |
| 1852. | PCK2 | NM_004563.2 | −1.7757677 | 0.419430945 | 1.48E−13 | 0.000463907 | 0 | 0.00303 |
| 1853. | C4orf18 | NM_016613.4 | −1.78113506 | −0.51090632 | 2.25E−15 | 3.56E−06 | 0 | 5.00E−05 |
| 1854. | FILIP1L | NM_014890.2 | −1.78355016 | 0.065279548 | 2.68E−15 | 0.430518135 | 0 | 0.62188 |
| 1855. | NACC2 | NM_144653.3 | −1.78707907 | 0.982252798 | 2.29E−13 | 8.40E−09 | 0 | 0 |
| 1856. | HLA-C | NM_002117.4 | −1.78750311 | 0.601528437 | 3.92E−15 | 5.72E−07 | 0 | 1.00E−05 |
| 1857. | MLPH | NM_001042467.1 | −1.78774866 | 0.423338475 | 4.98E−16 | 1.54E−05 | 0 | 0.00017 |
| 1858. | HLA-H | NR_001434.1 | −1.79752625 | 0.327002482 | 2.15E−13 | 0.004950496 | 0 | 0.02194 |
| 1859. | DAB2 | NM_001343.2 | −1.80688296 | 0.10922795 | 2.24E−15 | 0.194908618 | 0 | 0.36965 |
| 1860. | FST | NM_013409.1 | −1.81247271 | 0.910729653 | 2.67E−13 | 4.04E−08 | 0 | 0 |
| 1861. | TMEM173 | NM_198282.1 | −1.81647662 | 0.367287652 | 1.95E−16 | 5.70E−05 | 0 | 0.00052 |
| 1862. | NFIL3 | NM_005384.2 | −1.82334379 | 0.319541587 | 4.08E−13 | 0.008116955 | 0 | 0.03295 |
| 1863. | ATF5 | NM_012068.3 | −1.82638637 | 0.502456405 | 1.13E−13 | 8.02E−05 | 0 | 0.00069 |
| 1864. | MGC16121 | XM_001128419.1 | −1.82844552 | 0.013038775 | 9.76E−12 | 0.920850677 | 0 | 0.96188 |
| 1865. | DKFZp451A211 | NM_001003399.1 | −1.83289652 | 0.308656177 | 5.02E−15 | 0.001905623 | 0 | 0.00989 |
| 1866. | GALNTL2 | NM_054110.3 | −1.834124 | 0.988093636 | 2.27E−13 | 1.15E−08 | 0 | 0 |
| 1867. | KIAA1618 | NM_020954.2 | −1.8432906 | −0.2961344 | 3.46E−15 | 0.002381868 | 0 | 0.01192 |
| 1868. | TAPBP | NM_003190.3 | −1.84402904 | 0.474041086 | 5.63E−15 | 2.71E−05 | 0 | 0.00028 |
| 1869. | FADS1 | NM_013402.3 | −1.84532859 | 0.418638249 | 2.23E−15 | 6.64E−05 | 0 | 0.00058 |
| 1870. | MAMDC2 | NM_153267.3 | −1.8575359 | −0.02851138 | 1.76E−16 | 0.701012643 | 0 | 0.82965 |
| 1871. | IHPK3 | NM_054111.3 | −1.85853661 | −0.09088817 | 2.61E−15 | 0.294547496 | 0 | 0.48918 |
| 1872. | PFKFB4 | NM_004567.2 | −1.86485017 | 0.458182595 | 1.37E−16 | 3.94E−06 | 0 | 6.00E−05 |
| 1873. | SLC39A14 | NM_015359.2 | −1.87182882 | 0.480126518 | 5.22E−16 | 5.67E−06 | 0 | 8.00E−05 |
| 1874. | SRGN | NM_002727.2 | −1.87330953 | 0.203053161 | 7.72E−16 | 0.019424109 | 0 | 0.06604 |
| 1875. | ANGPTL2 | NM_012098.2 | −1.89297388 | −0.7837418 | 1.36E−13 | 4.40E−07 | 0 | 1.00E−05 |
| 1876. | APCDD1 | NM_153000.3 | −1.8940087 | −0.43013755 | 4.23E−16 | 2.32E−05 | 0 | 0.00024 |
| 1877. | TXNIP | NM_006472.2 | −1.90041037 | −0.01013871 | 2.39E−15 | 0.90723328 | 0 | 0.954 |
| 1878. | WARS | NM_173701.1 | −1.9174825 | 1.714743213 | 5.62E−14 | 4.46E−13 | 0 | 0 |
| 1879. | HMOX1 | NM_002133.1 | −1.92353063 | 0.983827625 | 2.97E−14 | 4.51E−09 | 0 | 0 |
| 1880. | RETSAT | NM_017750.2 | −1.92926221 | −0.17305505 | 6.12E−15 | 0.073845136 | 0 | 0.18451 |
| 1881. | HLA-F | NM_001098479.1 | −1.9323259 | 0.936849685 | 9.37E−14 | 2.91E−08 | 0 | 0 |
| 1882. | IGFBP4 | NM_001552.2 | −1.93713501 | 0.404758921 | 3.54E−14 | 0.000716137 | 0 | 0.00436 |
| 1883. | CFLAR | NM_003879.3 | −1.9400604 | 0.531255138 | 1.28E−14 | 2.13E−05 | 0 | 0.00023 |
| 1884. | SEMA4B | NM_198925.1 | −1.94044796 | 0.154164008 | 8.14E−15 | 0.115409463 | 0 | 0.25581 |
| 1885. | CYBASC3 | NM_153611.3 | −1.94506831 | −0.42179468 | 2.75E−14 | 0.000441848 | 0 | 0.0029 |
| 1886. | FTHL12 | NR_002205.1 | −1.94820294 | 0.387067696 | 1.85E−13 | 0.002386271 | 0 | 0.01194 |
| 1887. | IFITM3 | NM_021034.2 | −1.94910236 | −0.12744541 | 2.48E−16 | 0.118979489 | 0 | 0.26138 |
| 1888. | EVC | NM_014556.2 | −1.95418934 | −0.38572214 | 2.21E−14 | 0.000957476 | 0 | 0.00556 |
| 1889. | DGKA | NM_201554.1 | −1.9550059 | 0.10137545 | 3.22E−15 | 0.272321725 | 0 | 0.46432 |
| 1890. | SLC2A5 | NM_003039.1 | −1.95777901 | 0.814100831 | 7.46E−17 | 7.22E−10 | 0 | 0 |
| 1891. | IRF7 | NM_004029.2 | −1.95885807 | 1.13712661 | 1.53E−15 | 3.45E−11 | 0 | 0 |
| 1892. | TP53I3 | NM_147184.1 | −1.96051288 | 0.192047122 | 1.30E−15 | 0.036948411 | 0 | 0.10892 |
| 1893. | UNC93B1 | NM_030930.2 | −1.96070175 | 0.941069865 | 1.82E−15 | 1.09E−09 | 0 | 0 |
| 1894. | SPATA18 | NM_145263.2 | −1.98912505 | −0.21396871 | 7.38E−16 | 0.019986924 | 0 | 0.06752 |
| 1895. | CMBL | NM_138809.3 | −1.99166846 | 0.068582461 | 3.21E−17 | 0.350993311 | 0 | 0.54746 |
| 1896. | CXCL6 | NM_002993.2 | −1.99425721 | 0.172815354 | 3.56E−12 | 0.210855125 | 0 | 0.39011 |
| 1897. | APBB3 | NM_133172.2 | −1.99435525 | 0.039922542 | 1.93E−16 | 0.618738821 | 0 | 0.77355 |
| 1898. | IFI16 | NM_005531.1 | −2.00849219 | 0.684642292 | 1.91E−16 | 4.22E−08 | 0 | 0 |
| 1899. | APOBEC3G | NM_021822.1 | −2.01198251 | 1.407709172 | 7.16E−15 | 5.26E−12 | 0 | 0 |
| 1900. | FTH1 | NM_002032.2 | −2.0286533 | −0.41828197 | 9.42E−15 | 0.000413762 | 0 | 0.00275 |
| 1901. | ZBTB16 | NM_001018011.1 | −2.03660522 | 0.492114038 | 2.55E−14 | 0.000139081 | 0 | 0.00108 |
| 1902. | CES2 | NM_003869.4 | −2.04258122 | 0.061059702 | 3.02E−16 | 0.469468762 | 0 | 0.65591 |
| 1903. | PYGB | NM_002862.3 | −2.0462697 | −0.11799496 | 1.70E−16 | 0.158375594 | 0 | 0.31993 |
| 1904. | PARP10 | XM_001127571.1 | −2.06434441 | 0.542391889 | 2.19E−16 | 2.21E−06 | 0 | 3.00E−05 |
| 1905. | MT1E | NM_175617.3 | −2.06744305 | 0.128921886 | 5.64E−16 | 0.152396034 | 0 | 0.31151 |
| 1906. | C14orf159 | NM_024952.5 | −2.06956417 | 0.519442851 | 2.48E−15 | 2.13E−05 | 0 | 0.00023 |
| 1907. | ATOH8 | NM_032827.4 | −2.07341763 | −0.28861999 | 3.95E−15 | 0.007189922 | 0 | 0.02986 |
| 1908. | FTHL11 | NR_002204.1 | −2.08544399 | 0.60036409 | 5.37E−11 | 0.001480252 | 0 | 0.008 |
| 1909. | SCHIP1 | NM_014575.2 | −2.09276293 | 0.062627023 | 7.81E−16 | 0.490657209 | 0 | 0.67346 |
| 1910. | SNAI2 | NM_003068.3 | −2.09474629 | 0.202739617 | 5.30E−17 | 0.016707696 | 0 | 0.0586 |
| 1911. | C20orf127 | NM_080757.1 | −2.097565 | 0.440084688 | 2.49E−15 | 0.000168115 | 0 | 0.00128 |
| 1912. | PARP9 | NM_031458.1 | −2.09823879 | 0.44258682 | 4.51E−17 | 1.35E−05 | 0 | 0.00016 |
| 1913. | LOC441019 | XM_498969.2 | −2.10656054 | 0.262468867 | 1.27E−16 | 0.004402357 | 0 | 0.01994 |
| 1914. | ANPEP | NM_001150.1 | −2.1180096 | 0.363474839 | 2.43E−16 | 0.000376437 | 0 | 0.00254 |
| 1955. | H1F0 | NM_005318.2 | −2.4337012 | 0.50036236 | 6.56E−18 | 5.13E−06 | 0 | 7.00E−05 |
| 1956. | CEBPB | NM_005194.2 | −2.44089558 | 0.278291436 | 9.23E−18 | 0.003066339 | 0 | 0.01472 |
| 1957. | MT1X | NM_005952.2 | −2.44392836 | 0.31620115 | 1.29E−16 | 0.003343988 | 0 | 0.01584 |
| 1958. | XPC | NM_004628.3 | −2.45802741 | 0.262855334 | 3.54E−17 | 0.007993295 | 0 | 0.03257 |
| 1959. | DDX58 | NM_014314.3 | −2.46609401 | 0.021135353 | 9.80E−17 | 0.824829801 | 0 | 0.90712 |
| 1960. | CXCL5 | NM_002994.3 | −2.46876181 | 0.824027488 | 2.93E−17 | 1.24E−08 | 0 | 0 |
| 1961. | SLC7A2 | NM_001008539.2 | −2.48246747 | −0.10549691 | 4.56E−18 | 0.206903447 | 0 | 0.38475 |
| 1962. | USP18 | NM_017414.3 | −2.49706957 | 0.995965697 | 3.08E−15 | 3.57E−08 | 0 | 0 |
| 1963. | C9orf169 | NM_199001.1 | −2.50942513 | −0.05213055 | 6.67E−16 | 0.628407413 | 0 | 0.78053 |
| 1964. | TRIM25 | NM_005082.4 | −2.51325039 | 0.513858248 | 1.02E−18 | 1.48E−06 | 0 | 2.00E−05 |
| 1965. | | BQ437417 | −2.51406874 | −0.12597104 | 1.95E−16 | 0.220628257 | 0 | 0.40212 |
| 1966. | CCL5 | NM_002985.2 | −2.52794377 | 1.888757888 | 2.25E−16 | 5.51E−14 | 0 | 0 |
| 1967. | SAMD9L | NM_152703.2 | −2.53219161 | 1.257342146 | 4.23E−16 | 1.63E−10 | 0 | 0 |
| 1968. | UBA7 | NM_003335.2 | −2.56074458 | 0.572893137 | 1.30E−18 | 4.87E−07 | 0 | 1.00E−05 |
| 1969. | FTHL3 | NR_002201.1 | −2.57708257 | 0.449767394 | 1.95E−12 | 0.014144559 | 0 | 0.05134 |
| 1970. | TRIM22 | NM_006074.3 | −2.58070903 | −0.01964607 | 1.84E−18 | 0.80896617 | 0 | 0.89788 |
| 1971. | PRIC285 | NM_033405.2 | −2.60751113 | 0.86339373 | 4.43E−16 | 1.31E−07 | 0 | 0 |
| 1972. | AGRN | NM_198576.2 | −2.61234898 | 0.380803939 | 5.27E−17 | 0.000831554 | 0 | 0.00495 |
| 1973. | CA12 | NM_001218.3 | −2.62949301 | 0.954030803 | 1.09E−17 | 1.33E−09 | 0 | 0 |
| 1974. | C1Oorf10 | NM_007021.2 | −2.64142371 | 0.143168554 | 1.70E−16 | 0.18362568 | 0 | 0.35433 |
| 1975. | IRF1 | NM_002198.1 | −2.64332853 | 0.356258026 | 1.24E−16 | 0.002391579 | 0 | 0.01195 |
| 1976. | LOC729009 | XR_042330.1 | −2.65422979 | 0.663393857 | 1.60E−13 | 0.000274766 | 0 | 0.00194 |
| 1977. | CCL2 | NM_002982.3 | −2.68131723 | 0.435521389 | 3.91E−17 | 0.000249721 | 0 | 0.00179 |
| 1978. | STAT2 | NM_005419.2 | −2.69031186 | 0.18606978 | 7.23E−18 | 0.05146687 | 0 | 0.14058 |
| 1979. | CHI3L2 | NM_004000.2 | −2.70474249 | 0.257337616 | 1.06E−15 | 0.040179604 | 0 | 0.11653 |
| 1980. | OAS2 | NM_002535.2 | −2.71119798 | 0.455321205 | 6.79E−18 | 6.43E−05 | 0 | 0.00057 |
| 1981. | TNFRSF14 | NM_003820.2 | −2.72030488 | 0.566641253 | 2.54E−17 | 1.08E−05 | 0 | 0.00013 |
| 1982. | PTX3 | NM_002852.2 | −2.74410345 | 0.944168451 | 1.49E−15 | 1.93E−07 | 0 | 0 |
| 1983. | HLA-B | NM_005514.5 | −2.75247113 | 0.336698468 | 3.15E−18 | 0.001076552 | 0 | 0.00612 |
| 1984. | PARP14 | NM_017554.1 | −2.82549264 | 0.521139258 | 8.29E−16 | 0.000349183 | 0 | 0.00237 |
| 1985. | C1R | NM_001733.4 | −2.82916155 | 0.493314318 | 1.56E−17 | 6.87E−05 | 0 | 6.00E−04 |
| 1986. | DHX58 | NM_024119.2 | −2.83610757 | 0.696456542 | 5.70E−18 | 3.83E−07 | 0 | 1.00E−05 |
| 1987. | SAMD9 | NM_017654.2 | −2.86330823 | 1.398759917 | 4.03E−16 | 2.07E−10 | 0 | 0 |
| 1988. | TNFAIP3 | NM_006290.2 | −2.89673376 | 0.279310424 | 1.94E−19 | 0.002373549 | 0 | 0.01189 |
| 1989. | STAT1 | NM_007315.2 | −2.91526323 | 0.823534962 | 5.93E−19 | 6.91E−09 | 0 | 0 |
| 1990. | MT1M | NM_176870.2 | −2.92124936 | 1.185124452 | 2.90E−18 | 5.89E−11 | 0 | 0 |
| 1991. | ISG20 | NM_002201.4 | −2.93954241 | 2.357383735 | 8.62E−16 | 5.49E−14 | 0 | 0 |
| 1992. | SP110 | NM_004510.2 | −2.94061725 | 1.154252385 | 1.23E−18 | 4.82E−11 | 0 | 0 |
| 1993. | TMEM140 | NM_018295.2 | −2.94612466 | 1.003497903 | 2.66E−18 | 1.12E−09 | 0 | 0 |
| 1994. | MLKL | NM_152649.1 | −2.99195612 | 1.457088074 | 1.17E−18 | 9.49E−13 | 0 | 0 |
| 1995. | NFKBIA | NM_020529.1 | −3.00447087 | 0.275123837 | 8.22E−19 | 0.006082319 | 0 | 0.026 |
| 1996. | VCAM1 | NM_001078.2 | −3.01371818 | 0.634904663 | 9.62E−20 | 1.61E−07 | 0 | 0 |
| 1997. | UBE2L6 | NM_004223.3 | −3.08486585 | 0.580836267 | 5.31E−20 | 5.41E−07 | 0 | 1.00E−05 |
| 1998. | PSMB9 | NM_002800.4 | −3.13725921 | 0.945082183 | 1.75E−19 | 8.27E−10 | 0 | 0 |
| 1999. | PARP12 | NM_022750.2 | −3.16502003 | 0.776741185 | 5.52E−20 | 9.29E−09 | 0 | 0 |
| 2000. | HERC5 | NM_016323.2 | −3.20215105 | 1.329051901 | 5.85E−19 | 8.98E−12 | 0 | 0 |
| 2001. | LY6E | NM_002346.1 | −3.25130872 | 0.248387222 | 1.22E−18 | 0.020878793 | 0 | 0.06983 |
| 2002. | TAP1 | NM_000593.5 | −3.26471648 | 0.699574809 | 5.16E−20 | 7.58E−08 | 0 | 0 |
| 2003. | VWCE | NM_152718.2 | −3.31730424 | −0.12131841 | 3.29E−20 | 0.162954271 | 0 | 0.32605 |
| 2004. | CXCL1 | NM_001511.1 | −3.52334103 | 0.202825552 | 4.87E−15 | 0.234452652 | 0 | 0.41911 |
| 2005. | XAF1 | NM_199139.1 | −3.62201153 | 0.622248884 | 1.55E−19 | 4.24E−06 | 0 | 6.00E−05 |
| 2006. | IFIH1 | NM_022168.2 | −3.70470773 | 1.658871536 | 3.25E−21 | 1.61E−14 | 0 | 0 |
| 2007. | HERC6 | NM_017912.3 | −3.72816842 | 0.523690476 | 2.27E−21 | 3.40E−06 | 0 | 5.00E−05 |
| 2008. | SLC15A3 | NM_016582.1 | −3.75445448 | 0.775468832 | 6.30E−20 | 1.56E−07 | 0 | 0 |
| 2009. | C1QTNF1 | NM_198594.1 | −3.84595088 | 0.769631513 | 6.55E−22 | 6.27E−09 | 0 | 0 |
| 2010. | IFI35 | NM_005533.2 | −3.84734525 | 0.916890243 | 1.15E−19 | 2.74E−08 | 0 | 0 |
| 2011. | IFIT3 | NM_001549.2 | −3.85736134 | 1.806045196 | 4.38E−21 | 9.31E−15 | 0 | 0 |
| 2012. | IL8 | NM_000584.2 | −3.88478909 | 1.186102426 | 4.85E−19 | 1.60E−09 | 0 | 0 |
| 2013. | OAS3 | NM_006187.2 | −4.02902003 | 0.865423871 | 2.43E−20 | 3.98E−08 | 0 | 0 |
| 2014. | MX2 | NM_002463.1 | −4.06614449 | 0.957591482 | 9.83E−20 | 2.91E−08 | 0 | 0 |
| 2015. | LOC100129681 | XM_001726834.1 | −4.07488433 | 0.518860683 | 6.22E−20 | 9.44E−05 | 0 | 0.00078 |
| 2016. | EPSTI1 | NM_033255.2 | −4.12032125 | 0.661837944 | 5.41E−23 | 2.76E−08 | 0 | 0 |
| 2017. | SAA1 | NM_199161.1 | −4.13820981 | 0.256850133 | 9.91E−22 | 0.008096703 | 0 | 0.03289 |
| 2018. | IFI6 | NM_022872.2 | −4.24031026 | −0.0361139 | 6.20E−22 | 0.683302757 | 0 | 0.81802 |
| 2019. | BST2 | NM_004335.2 | −4.24809874 | 0.21003593 | 1.36E−22 | 0.017358257 | 0 | 0.06038 |
| 2020. | ECGF1 | NM_001953.2 | −4.29858283 | 0.615594642 | 2.23E−22 | 4.84E−07 | 0 | 1.00E−05 |
| 2021. | ISG15 | NM_005101.1 | −4.31057423 | 0.127991971 | 3.24E−22 | 0.151450311 | 0 | 0.31003 |
| 2022. | IFIT2 | NM_001547.4 | −4.31465849 | 2.069685191 | 1.68E−20 | 2.16E−14 | 0 | 0 |
| 2023. | IFIT1 | NM_001548.3 | −4.37943573 | 0.720554258 | 1.96E−21 | 3.24E−07 | 0 | 1.00E−05 |
| 2024. | OAS1 | NM_001032409.1 | −4.60699105 | 1.453177095 | 7.12E−20 | 1.68E−10 | 0 | 0 |
| 2025. | SOD2 | NM_001024466.1 | −4.61702534 | 1.53216666 | 2.97E−22 | 4.31E−13 | 0 | 0 |
| 2026. | IFI44L | NM_006820.1 | −5.11742006 | 0.46513265 | 2.83E−23 | 4.85E−05 | 0 | 0.00045 |
| 2027. | CFB | NM_001710.4 | −5.6232501 | 0.633698868 | 1.91E−24 | 4.54E−07 | 0 | 1.00E−05 |
| 2028. | MX1 | NM_002462.2 | −5.67457065 | 0.26243156 | 4.15E−25 | 0.003903492 | 0 | 0.01805 |
| 2029. | IFITM1 | NM_003641.3 | −6.11814111 | 0.344079828 | 2.80E−24 | 0.001806122 | 0 | 0.00947 |
| 2030. | IFI27 | NM_005532.3 | −6.52653374 | 0.274602374 | 1.66E−26 | 0.002273497 | 0 | 0.01148 |

*Each gene sequence in Table 1, as identified by the Genbank reference number accessed on Jul. 22, 2011, is hereby incorporated herein by reference.

While the preferred embodiment of the invention has been illustrated and described, it will be appreciated that various changes can be made therein without departing from the spirit and scope of the invention.

### REFERENCES

The following references, to the extent that they provide exemplary procedural or other details supplementary to those set forth herein, are specifically incorporated herein by reference:
U.S. Pat. No. 4,376,110
U.S. Pat. No. 5,091,513
U.S. Pat. No. 6,506,559
U.S. Pat. No. 6,573,099
U.S. Pat. No. 6,881,557
U.S. Patent Publn. 2002/0168707
U.S. Patent Publn. 2003/0051263
U.S. Patent Publn. 2003/0055020
U.S. Patent Publn. 2003/0159161
U.S. Patent Publn. 2004/0064842
U.S. Patent Publn. 2004/0265839
U.S. Patent Publn. 2005/0214860
Altschul et al., Nucleic Acids Res., 25:3389-3402, 1997.
Ausubel et al., In: Current Protocols in Molecular Biology, Vol. 2, Current Protocols Publishing, NY, 1994.
Belancio et al., Semin. Cancer Biol., 20:200-210, 2010.
Bonnefont, Am. J. Hum. Genet., 83:208-218, 2008.
Booth and Holland Gene, 387:7-14, 2007.
Bosch et al., Hum. Mol. Genet., 16:2572-2582, 2007.
Boussif et al., Proc. Natl. Acad. Sci. USA, 92(16):7297-7301, 1995.
Candille et al., Science, 318:1418-1423, 2007.
Cao et al., Dev. Cell, 18:662-674, 2010.
Chang et al., PloS One, 6:e16867, 2011.
Chen and Okayama, Mol. Cell. Biol., 7(8):2745-2752, 1987.
Clapp et al., Am. J. Hum. Genet., 81:264-279, 2007.
Clapp et al., Am. J. Hum. Genet., 81:264-279, 2007.
Coligan et al., Current Protocols in Immunology, 1(2): Chapter 5, 1991.
de Greef et al., Hum. Mutat., 30:1449-1459, 2009.
Dixit et al., Proc. Natl. Acad. Sci. USA, 104:18157-18162, 2007.
Du et al., Bioinformatics, 24:1547-1548, 2008.
Falcon and Gentleman, Bioinformatics, 23:257-258, 2007.
Fechheimer et al., Proc. Natl. Acad. Sci. USA, 84:8463-8467, 1987.
Feng et al., J. Immunol., 177:782-786, 2006.
Fraley et al., Proc. Natl. Acad. Sci. USA, 76:3348-3352, 1979.
Frisullo et al., J. Clin. Immunol., 31:155-166, 2011.
Gabellini et al., Cell, 110:339-348, 2002.
Gabellini et al., Nature, 439:973-977, 2006.
Gabriels et al., Gene, 236:25-32, 1999.
Geng et al., Hybridoma Larchmt., 30:125-130, 2011.
Gopal, Mol. Cell Biol., 5:1188-1190, 1985.
Graham and Van Der Eb, Virology, 52:456-467, 1973.
Griffin et al., J. Cell Sci., 123:3052-3060, 2010.
Harland and Weintraub, J. Cell Biol., 101(3):1094-1099, 1985.
Holland et al., BMC Biol., 5:47, 2007.
Hu et al, Cancer Res., 56:3055-3061, 1996.
Innis et al., In: PCR Protocols: A Guide to Methods and Applications, Academic Press Inc., San Diego Calif., 1990.
Jagasia et al., Cancer Letters, 105:91-103, 1996.
Jiang et al., Genomics, 80:621-629, 2002.
Jin et al., Dev. Dyn., 237:3435-3443, 2008.
Jin et al., PloS One, 5e10993, 2010.
Kawamura-Saito et al., Hum. Mol. Genet., 15:2125-2137, 2006.
Klooster et al., Eur. J. Hum. Genet., 17:1615-1624, 2009.
Knoepfler et al., Nucleic Acids Res., 27:3752-3761, 1999.
Köhler and Milstein, Eur. J. Immunol., 6:511-519, 1976.
Köhler and Milstein, Nature, 256(5517):495-497, 1975.
Köhler et al., Eur. J. Immunol., 6(4):292-295, 1976.
Kowaljow et al., Neuromuscul. Discord., 17:611-623, 2007.
Kowaljow et al., Neuromuscul. Disord., 17:611-623, 2007.
Lai and Gallo, Trends Immunol., 30:131-141, 2009.
Laoudj-Chemvesse et al., J. Mol. Med., 83:216-224, 2005.
Lemmers et al., Science, 329:1650-1653, 2010.
Lyle et al., Genomics, 28:389-397, 1995.
Melchionna et al., Muscle Nerve, 41:828-835, 2010.
Midorikawa et al., Infect. Immun., 71:3730-3739. 2003.
Molnar et al., Eur. Arch. Psychiatry Clin. Neuroscie., 241:105-108, 1991.
Monoclonal Antibodies and T-Cell Hybridomas, Elsevier, N.Y., 563-681, 1981.
Nicolau and Sene, Biochim. Biophys. Acta, 721:185-190, 1982.
Palii et al., EMBO J., 30:494-509, 2011.
PCT Appln. PCT/US2011/048318
PCT Appln. WO/2012/024535,
Pierre et al., Genomics, 90:583-594, 2007.
Potter et al., Proc. Natl. Acad. Sci. USA, 81:7161-7165, 1984.
Reed et al., Exp. Neurol., 205: 583-586, 2007.
Rippe, et al., Mol. Cell Biol., 10:689-695, 1990.
Sambrook et al., In: Molecular Cloning: A Laboratory Manual (2nd ed.), Vols. 1-3, Cold Spring Harbor Laboratory, Cold Spring Harbor, N.Y., 1989.
Sandling et al., Eur. J. Hum. Genet., 19(4):479-84, 2011.
Sass et al., Infect. Immun., 78:2793-2800, 2010.
Schulz et al., J. Biomed. Biotechnol., 83672, 2006.
Semple et al., Eur. J. Immunol., 40:1073-1078, 2010.
Semple et al., Eur. J. Immunol., 41(11):3291-3300, 2011.
Simpson et al., Nat. Rev. Cancer, 5:615-625, 2005.
Smit, Nucleic Acids Res., 21:1863-1872, 1993.
Snider et al., PloS Genet, 6:e1001181, 2010.
Stanghellini et al., Gene Expr. Patterns, 9:595-602, (2009.
Sun et al., Proc. Natl. Acad. Sci. USA, 95:10009-10014, 1998.
Tatiana et al., FEMS Microbiol. Lett., 174:247-250, 1999.
Trompeter et al, J. Immunol. Methods, 274(1-2):245-256, 2003.
Tuplet et al., J. Med. Genet., 33:366-370, 1996.
Tur-Kaspa et al., Mol. Cell Biol., 6:716-718, 1986.
van Overveld et al., Nat. Genet., 35:315-317, 2003.
Wallace et al., Ann. Neurol., 69:540-552, 2011.
Wallace et al., Ann. Rev. Neurol., 69(3):540-52, 2011.
Wettenhall and Smyth, Bioinformatics, 20:3705-3706, 2004.
Wijmenga et al., Nat. Genet., 2:26-30, 1992.
Wootton and Federhen, Methods in Enzymology, 266:554-571, 1996.
Wu and Wu, Biochemistry, 27: 887-892, 1988.
Wu and Wu, J. Biol. Chem., 262:4429-4432, 1987.
Wu et al., Duxbl Dev. Dyn., 239:927-940, 2010.
Wuebbles et al., Int. J. Clin. Exp. Pathol., 3:386-400, 2010.
Yang et al. Proc. Natl. Acad. Sci. USA, 87:4144-4148, 1990.
Yang et al., Science, 286:525-528, 1999.
Zalzman et al., Nature, 464:858-863, 2010.
Zeng et al., PloS Genet 5, e1000559, 2009.

1. A method of increasing or maintaining the innate immune response in a mammalian subject in need thereof, comprising administering to the mammalian subject an agent capable of inhibiting, or suppressing the level of DEFB103A and/or DEFB103B expression in a population of cells in the mammalian subject.

2. The method of claim 1, wherein the agent specifically binds to DEFB103 polypeptide or a nucleic acid encoding DEFB103 polypeptide.

3. The method of claim 1, wherein the agent is a DEFB103 antibody.

4. (canceled)

5. The method of either of claim 1, wherein the agent is a DEFB103A-specific or DEFB103B-specific siRNA.

6. The method of claim 1, wherein the agent is capable of inhibiting DUX4-fl mediated transcriptional activation.

7. The method of claim 1, wherein the subject in need thereof is selected from the group consisting of (i) a subject suffering from, or at risk for developing FSHD, (ii) a subject suffering from or at risk for developing myotonic dystrophy, (iii) a subject suffering from or at risk for developing Huntington's disease, (iv) a subject suffering from cancer, (v) a subject suffering from an autoimmune disease, and (vi) a subject infected with a pathogen, such as a virus, such as HIV.

8. A method of suppressing or inhibiting the innate immune response in a mammalian subject in need thereof, comprising administering to the mammalian subject an agent capable of inducing, or increasing the level of DEFB103A and/or DEFB103B expression or activity in a population of cells in the mammalian subject.

9-17. (canceled)

18. A method of promoting myogenesis in muscle cells comprising contacting the cells with an agent capable of inhibiting DEFB103 expression and/or DEFB103 activity.

19-25. (canceled)

25. A method of promoting or maintaining muscle differentiation in a mammalian subject in need thereof, comprising administering to the mammalian subject an agent capable of inhibiting, or suppressing the level of DEFB103 expression and/or inhibiting DEFB103 activity in a population of muscle cells in the mammalian subject.

26-32. (canceled)

33. A method of inhibiting the innate immune response in a mammalian subject in need thereof, comprising administering to the mammalian subject an agent capable of inducing, or increasing the level of DUX4-fl expression in a population of cells in the mammalian subject.

34-46. (canceled)

47. A method of increasing or maintaining the innate immune response in a mammalian subject in need thereof, comprising administering to the mammalian subject an agent capable of inhibiting, or suppressing the level of DUX4-fl expression, or an agent capable of inhibiting DUX4-fl mediated transcription activation in a population of cells in the mammalian subject, wherein the mammalian subject does not suffer from, or at risk for Facioscapulohumeral Destrophy (FSHD).

48-59. (canceled)

60. A method of inducing one or more testis expressed genes in a non-testis cell type comprising contacting the non-testis cell type with an agent capable of inducing, or increasing the level of DUX4-fl expression in a population of cells.

61-68. (canceled)

69. A method of determining the presence of, or risk of developing, Facioscapulohumeral dystrophy (FSHD) in a mammalian subject, comprising:
(a) determining the presence or amount of at least one FSHD biomarker in a biological test sample obtained from a mammalian subject, wherein the at least one FHSD biomarker comprises a gene product of a DUX-4-fl induced gene; and
(b) comparing the presence or amount of the biomarker determined in step (a) with a reference standard or control sample,
wherein an increase in the presence or amount of the FSHD biomarker determined in the test sample in comparison to the reference standard or control sample is indicative of the presence of FSHD, or increased risk of developing FSHD, in the mammalian subject.

70-88. (canceled)

89. An isolated polynucleotide probe for detecting an FSHD biomarker, or a polynucleotide primer for amplifying at least a portion of an FSHD biomarker, wherein the nucleic acid probe or primer has a length of from at least 10 nucleotides to 200 nucleotides and specifically hybridizes to the nucleic acid sequence of at least two FSHD biomarker set forth in TABLE 1 or TABLE 2.

90. An isolated population of polynucleotide probes comprising a plurality of polynucleotides each complementary and hybridizable to a sequence of at least two different FSHD biomarkers selected from any one of TABLE 1 or TABLE 2.

91-93. (canceled)

94. An isolated populations of antibodies that specifically bind to at least two different FSHD polypeptide biomarkers encoded by nucleic acids set forth in TABLE 1 or TABLE 2.

95-97. (canceled)

98. A kit comprising one or more detection reagents for detecting one or more FSHD biomarkers set forth in TABLE 1 or TABLE 2 for use in an assay to determine the presence or risk of FSHD in a biological sample obtained from a mammalian subject.

99. A nucleic acid molecule comprising an expression cassette comprising a promoter operationally linked to a reporter gene or selectable marker, wherein the promoter contains at least one DUX4-responsive element comprising the consensus sequence “TAAYBBAATCA” (SEQ ID NO:166).

100-101. (canceled)

102. A method of detecting the presence of DUX4-fl protein in a cell sample comprising introducing a nucleic acid molecule comprising an expression cassette comprising a promoter operationally linked to a reporter gene or selectable marker, wherein the promoter contains at least one DUX4-responsive element comprising the consensus sequence “TAAYBBAATCA” (SEQ ID NO:166), and assaying the cell for expression of the reporter gene, or selecting for growth under conditions requiring expression of the selectable marker.

103. A method of identifying an inhibitor of DUX4-fl induced expression comprising:
(a) contacting a cell containing:
(i) a nucleic acid molecule comprising an expression cassette comprising a promoter operationally linked to a reporter gene or selectable marker, wherein the promoter contains at least one DUX4-responsive element comprising the consensus sequence “TAAYBBAATCA” (SEQ ID NO:166), and
(ii) DUX4-fl polypeptide,
with a candidate inhibitory agent; and
(b) determining whether the cell expresses the reporter gene or selectable marker in the presence and absence of the candidate inhibitory agent,
wherein the absence of expression of the reporter gene or selectable marker in the presence of the inhibitory agent indicates that the agent is an inhibitor of DUX4-fl induced expression.

104-105. (canceled)
